(19) United States
(12) Patent Application Publication
(10) Pub. No.: US 2023/0151020 A1
(43) Pub. Date: May 18, 2023

(54) HETEROCYCLIC COMPOUND AND ORGANIC ELECTROLUMINESCENT DEVICE

(71) Applicants: WUHAN TIANMA MICROELECTRONICS CO., LTD., Wuhan (CN); WUHAN TIANMA MICROELECTRONICS CO., LTD. SHANGHAI BRANCH, Shanghai (CN)

(72) Inventors: Wei GAO, Shanghai (CN); Wenpeng DAI, Shanghai (CN); Lu ZHAI, Shanghai (CN); TINGTING LU, Shanghai (CN); Lilian KUANG, Shanghai (CN)

(73) Assignees: WUHAN TIANMA MICROELECTRONICS CO., LTD., Wuhan (CN); WUHAN TIANMA MICROELECTRONICS CO., LTD. SHANGHAI BRANCH, Shanghai (CN)

(21) Appl. No.: 18/155,777

(22) Filed: Jan. 18, 2023

(30) Foreign Application Priority Data
Jul. 18, 2022 (CN) 202210841904.1

(51) Int. Cl. C07D 491/048; C07D 491/147; C07D 495/04; C07D 495/14; H10K 85/60

(52) U.S. Cl. CPC C07D 491/048; C07D 491/147; C07D 495/04; C07D 495/14; H10K 85/657

(57) ABSTRACT

The present disclosure provides a heterocyclic compound and an organic electroluminescence device. The heterocyclic compound has a structure as shown in formula I. In the heterocyclic compound provided by the present disclosure, the nitrogen atoms in Hy₁ and Hy₁ can have excellent doping effect with metals or metal-organic complexes to form a stable binding effect, to inhibit the metal movement during the working process of the device and forming a more stable hybrid film to improve the capabilities of electron injection and electron transport, and the holes and electrons in the device are balanced, to reduce the working voltage of the OLED device and improving the luminous efficiency and working lifetime thereof,

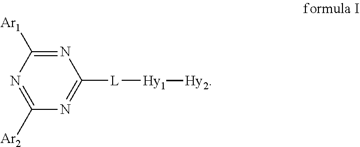

### CROSS REFERENCE TO RELATED APPLICATIONS

This application claims the priority of Chinese Patent Application No. 202210841904.1, filed with the China National Intellectual Property Administration on Jul. 18, 2022, and titled with “HETEROCYCLIC COMPOUND AND ORGANIC ELECTROLUMINESCENT DEVICE”, which is hereby incorporated by reference.

### FIELD

The present disclosure relates to the field of semiconductor materials, and in particular to a heterocyclic compound and an organic electroluminescent device.

### BACKGROUND

Generally, an organic electroluminescent device has a structure comprising an anode, a cathode, and an organic material layer located therebetween. Under a drive of an electric field, current carriers (holes and electrons) in an organic electroluminescent device are injected from the anode and cathode of the device, respectively, and recombined in the light-emitting layer to form excitons. The excitons decay through radiative transitions, returning to the ground state, and producing fluorescence or phosphorescence.

Organic materials used in OLED devices can be divided into two categories in terms of use, namely general materials and light-emitting materials. The light-emitting materials include host materials and guest materials (or doping materials); the general materials include hole injection materials, hole transport materials, electron blocking materials, hole blocking materials, electron transport materials, and electron injection materials.

In recent years, through the continuous efforts of developers, materials and technologies of OLEDs have been continuously optimized, and have been gradually commercialized. However, lower working voltage, higher efficiency, longer device lifetime, and more saturated luminescence spectrum are still the biggest challenge faced by the mass production and large-scale application of OLED. Therefore, selecting a combination of OLED materials with better performance is an important topic in the art at present.

Due to the different injection capabilities and transport rates of holes and electrons, the recombination region in the light-emitting layer is greatly offset, which is not conducive to the stability of the device. Therefore, how to adjust the balance of holes and electrons and how to adjust the recombination region have always been an important topic in the art.

In order to further improve the display quality of organic light-emitting devices, in addition to adjusting the structure of OLED devices, researchers are further seeking to change light-emitting materials and general materials to improve the transport efficiency of holes and electrons, and the holes and electrons in the device can be balanced, to improve the luminescence efficiency and lifetime of the OLED device. As a basic component of OLED devices, organic electron transport material assists the injection of electrons from the cathode to the light-emitting layer, while avoiding luminescence quenching caused by direct contact between the cathode and the light-emitting layer, which plays a crucial role in determining the efficiency and stability of OLED devices. Therefore, it is particularly important to develop a new electron transport material with high performance and long lifetime to better meet the performance requirements of organic electroluminescent devices.

### SUMMARY

In order to further improve the display quality of the organic electroluminescent device, the electron transport rate and the film stability of the electron transport layer, the present disclosure provides a heterocyclic compound having the structure as shown in formula I:

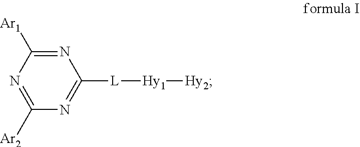

In one embodiment, Ar₁ and Ar₂ are independently selected from the group consisting of hydrogen, deuterium, substituted or unsubstituted C₆-C₁₈ aryl or C₂-C₁₈ heteroaryl;

L is selected from the group consisting of single bond, substituted or unsubstituted C₆-C₃₀ arylene;

Hy₁ and Hy₂ are independently selected from the structures represented by formula II or formula III, and Hy₁ and Hy₂ are different;

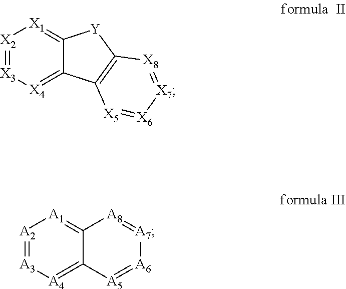

Y is 0 or S;

X₁, X₂, X₃, X₄, X₅, X₆, X₇, X₈, A₁, A₂, A₃, A₄, A₅, A₆, A₇ and A₈ are independently selected from the group consisting of N atom or CR₁; at least one of X₁, X₂, X₃, X₄, X₅, X₆, X₇ and X₈ is an N atom, and at least one of A₁, A₂, A₃, A₄, A₅, A₆, A₇ and A₈ is an N atom;

R₁ is selected from the group consisting of hydrogen, deuterium, substituted or unsubstituted C₁-C₆ alkyl or C₂-C₆ alkenyl, and adjacent R₁ may be connected to form a ring.

The present disclosure provides an organic electroluminescent device, and the organic electroluminescent device includes an anode, a cathode, and an organic thin film layer located between the anode and the cathode, the organic thin film layer includes an electron transport layer, and the electron transport layer includes at least one of the above-mentioned heterocyclic compounds.

The present disclosure provides an organic electroluminescent device, and the organic electroluminescent device includes an anode, a cathode, an organic thin film layer located between the anode and the cathode, and an organic cover layer located outside the cathode, and the organic cover layer includes at least one of the above-mentioned heterocyclic compounds.

The present disclosure provides a display device comprising the above organic electroluminescent device.

Compared with the prior art, the heterocyclic compound provided by the present disclosure has the following advantages:

In the heterocyclic compound provided by the present disclosure, the nitrogen atoms in Hy₁ and Hy₂ can have excellent doping effect with metals or metal-organic complexes to form a stable binding effect, to inhibit the metal movement during the working process of the device, and forming a more stable hybrid film to improve the capabilities of electron injection and electron transport, and the holes and electrons in the device can be balanced, to reduce the working voltage of the OLED device, and improving the luminous efficiency and working lifetime thereof.

### BRIEF DESCRIPTION OF DRAWINGS

FIG. 1 is the schematic structural diagram of the organic light-emitting device provided by the present disclosure.

### DETAILED DESCRIPTION

Embodiments of the present application will be further described in detail below with reference to the embodiments. It should be understood that the embodiments described in this specification are only for explaining the present application, but not for limiting the present application.

The above summary of the present application is not intended to describe each disclosed embodiment or each implementation in this application. The following description illustrates exemplary embodiments in more detail. In various places throughout this application, guidance is provided through a series of examples which can be used in various combinations. In each example, the enumeration is merely a representative group and should not be construed as exhaustion.

In the description herein, unless otherwise specified, “above” and “below” refers to the inclusion of the number per se, and the meaning of “more” in “one or more” refers to be above two.

The terms “a” and “the” both refer to one or more molecules of the compound but not a limitation of a single molecule of the compound. Furthermore, one or more of the molecules may be the same or different, so long as they fall within the scope of this chemical compound.

The term “comprising” and variations thereof when appear in the specification and claims have non-limiting sense.

The grouping of alternative elements or embodiments disclosed herein should not be construed as limiting. Members of each group may be employed and claimed individually or in any combination with other members of this group or other elements found herein. It can be contemplated that one or more members of a group may be included in or deleted from the group for reasons of convenience and/or patentability. When any such inclusion or deletion occurs, the specification is hereby deemed to be a group containing modification, thus satisfying the written description of all Markush groups used in the claims.

Unless otherwise stated, when a compound or chemical structural feature (such as an aryl) is referred to as being “substituted”, this feature may have one or more substituents. The term “substituent” has the broadest meaning known in the art, and includes a moiety which occupies the position that would normally be occupied by one or more hydrogen atoms attached to a parent compound or chemical structural feature.

The term “alkyl” not only includes straight or branched chain saturated hydrocarbyl, such as methyl, ethyl, propyl (such as n-propyl, isopropyl), butyl (such as n-butyl, isobutyl, sec-butyl, tert-butyl), pentyl (such as n-pentyl, isopentyl, neopentyl) and other similar alkyls, but also includes alkyl substituents with other substituents known in the art, and the other substituents are, for example, hydroxyl, alkylsulfonyl, halogen atoms, cyano, nitro, amino, carboxyl and the like. Thus, “alkyl” includes ether, haloalkyl, nitroalkyl, carboxyalkyl, hydroxyalkyl, sulfoalkyl and the like. In various embodiments, a C₁-C₂₀ alkyl is namely an alkyl having 1-20 carbon atoms.

The term “alkoxy” refers to —O-alkyl. Examples of alkoxy include, but are not limited to, methoxy, ethoxy, propoxy (such as n-propoxy, isopropoxy), butoxy (such as n-butoxy, isobutoxy, sec-butoxy, tert-butoxy) and other similar alkoxys.

The term “aryl” refers to a closed aromatic ring or ring system. Examples of aryl include, but are not limited to, phenyl, naphthyl, phenanthryl, anthracenyl, biphenyl, triphenylene, pyrenyl, spirobifluorenyl, chrysenyl, perylenyl, indenyl, and azulenyl. In various embodiments, a 6-30 membered aryl is namely an aryl having 6-30 carbon atoms to form a ring.

The term “heteroaryl” means that one or more atoms in the ring of an aryl is an element other than carbon (such as N, O, S). In some embodiments, the entirety of a 2-20 heteroaryl may have 1-6 or 1-3 ring heteroatoms (such as N, O, S). Examples of heteroaryl include, but are not limited to, pyrrolyl, furanyl, oxazolyl, isoxazolyl, thienyl, thiazolyl, isothiazolyl, thiadiazolyl, oxadiazolyl, imidazolyl, pyrazolyl, triazole, pyridazinyl, pyrazinyl, pyridyl, pyrimidinyl, triazinyl, indolyl, quinolinyl, isoquinolinyl, acridinyl, purinyl, pteridyl, benzofuranyl, benzothienyl, benzimidazolyl, benzothiazolyl, cinnoline, quinoxalinyl, dibenzofuranyl, dibenzothienyl and phenanthrolinyl. In various embodiments, a 2-20 membered aryl is namely an aryl having 2-20 atoms (including carbon atoms and heteroatoms) to form a ring.

The term “arylamino” refers to that the hydrogen on an aromatic ring is substituted with an amino. Examples of arylamino include, but are not limited to, cyanoarylamino, alkylarylamino, alkoxyarylamino, arylarylamino, and heteroarylarylamino.

The term “acridinyl” refers to a nitrogen-containing heterocycle having two benzene rings and one pyridine structure. Examples of acridinyl include, but are not limited to, cyanoacridinyl, alkylacridinyl (such as methylacridinyl, ethylacridinyl, propylacridinyl, and butylacridinyl), alkoxyacridinyl, arylcarbazolyl, heteroarylcarbazolyl, and arylaminocarbazolyl.

The term “carbazolyl” refers to a nitrogen-containing aromatic heterocycle. Examples of carbazolyl include, but are not limited to, cyanocarbazolyl, alkylcarbazolyl (such as methylcarbazolyl, ethylcarbazolyl, propylcarbazolyl, and butylcarbazolyl), alkoxycarbazolyl, arylcarbazolyl, heteroarylcarbazolyl, and arylaminocarbazolyl.

The term “hydrogen” refers to ¹H (protium, H), ²H (deuterium, D) or ³H (tritium, T). In various embodiments, “hydrogen” may be ¹H(protium,H).

At various places in this specification, substituents of compounds are disclosed in groups or ranges. This description is expressly contemplated to include each individual subcombination of members of these groups and ranges. For example, the term “C₁-C₈ alkyl” is expressly contemplated to individually disclose C₁, C₂, C₃, C₄, C₅, C₆, C₇, C₈, C₁-C₈, C₁-C₇, C₁-C₆, C₁-C₅, C₁-C₄, C₁-C₃, C₁-C₂, C₂-C₈, C₂-C₇, C₂-C₆, C₂-05, C₂-C₄, C₂-C₃, C₃-C₈, C₃-C₇, C₃-C₆, C₃-05, C₃-C₄, C₄-C₈, C₄-C₇, C₄-C₆, C₄-05, C₅-C₈, C₅-C₇, C₅-C₆, C₆-C₈, C₆-C₇ and C₇-C₈ alkyls. As other examples, integers ranging from 5 to 40 are expressly contemplated to individually disclose 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40; integers ranging from 1 to 20 are expressly contemplated to individually disclose 1, 2, 3, 4, 5, 6, 7, 8, 9, 10, 11, 12, 13, 14, 15, 16, 17, 18, 19, and 20. Accordingly, other groups or ranges can be expressly contemplated.

As used herein, the representation of a single bond crossing a single ring or multiple ring systems means that the single bond can be attached at any accessible position of the single ring or multiple ring systems.

Hereinafter, the present specification will be described in more detail.

The present disclosure provides a heterocyclic compound having the structure as shown in formula I:

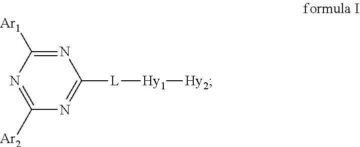

In one embodiment, Ar₁ and Ar₂ are independently selected from the group consisting of hydrogen, deuterium, substituted or unsubstituted C₆-C₁₈ aryl or C₂-C₁₈ heteroaryl;

L is selected from the group consisting of single bond, substituted or unsubstituted C₆-C₃₀ arylene;

Hy₁ and Hy₂ are independently selected from the structures represented by formula II or formula III, and Hy₁ and Hy₂ are different;

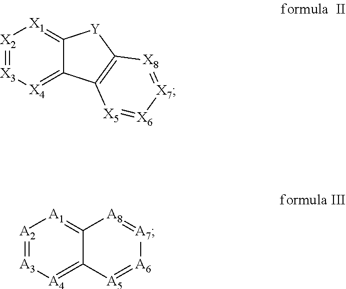

Y is 0 or S;

X₁, X₂, X₃, X₄, X₅, X₆, X₇, X₈, A₁, A₂, A₃, A₄, A₅, A₆, A₇ and A₈ are independently selected from the group consisting of N atom or CR₁; at least one of X₁, X₂, X₃, X₄, X₅, X₆, X₇, and X₈ is an N atom, and at least one of A₁, A₂, A₃, A₄, A₅, A₆, A₇, and A₈ is an N atom;

R₁ is selected from the group consisting of hydrogen, deuterium, substituted or unsubstituted C₁-C₆ alkyl or C₂-C₆ alkenyl, and adjacent R₁ may be connected to form a ring.

In one embodiment, the substituent of the C₆-C₁₈ aryl or the C₂-C₁₈ heteroaryl is one or more selected from the group consisting of deuterium, halogen, cyano, methyl, ethyl, isopropyl, tert-butyl, adamantyl, and triphenylsilyl;

the substituent of the C₆-C₃₀ arylene is one or more selected from the group consisting of deuterium, halogen, cyano, methyl, ethyl, isopropyl, tert-butyl, adamantyl, and triphenylsilyl;

the substituent of the C₁-C₆ alkyl or the C₂-C₆ alkenyl is one or more selected from the group consisting of deuterium, halogen, cyano, methyl, ethyl, isopropyl, tert-butyl, adamantyl, and triphenylsilane.

In one embodiment, the Ar₁ and Ar₂ are independently selected from the group consisting of hydrogen, deuterium, phenyl, biphenyl, naphthyl, anthracenyl, phenanthryl, thienyl, furanyl, benzothienyl, benzofuranyl, dibenzofuranyl, dibenzothienyl or pyridyl.

In some embodiments, Ar₁, Ar₂ are independently selected from the group consisting of hydrogen, deuterium, and phenyl.

In one embodiment, L is selected from the group consisting of single bond, phenylene, naphthylene, phenanthrylene, anthracenylene, pyrrolylidene, thienylene, furanylene, benzofuranylene, benzothienylene, dibenzofuranyl ene, dibenzothienylene, pyridinylene, pyrimidinylene, pyridazinylene, triazinylene, pyrazinylene, quinolinylene, quinoxalinylene or isoquinolinylene.

In some embodiments, L is selected from the group consisting of single bond, phenylene, and naphthylene.

In some embodiments, the above formula II has any one of the following structures:

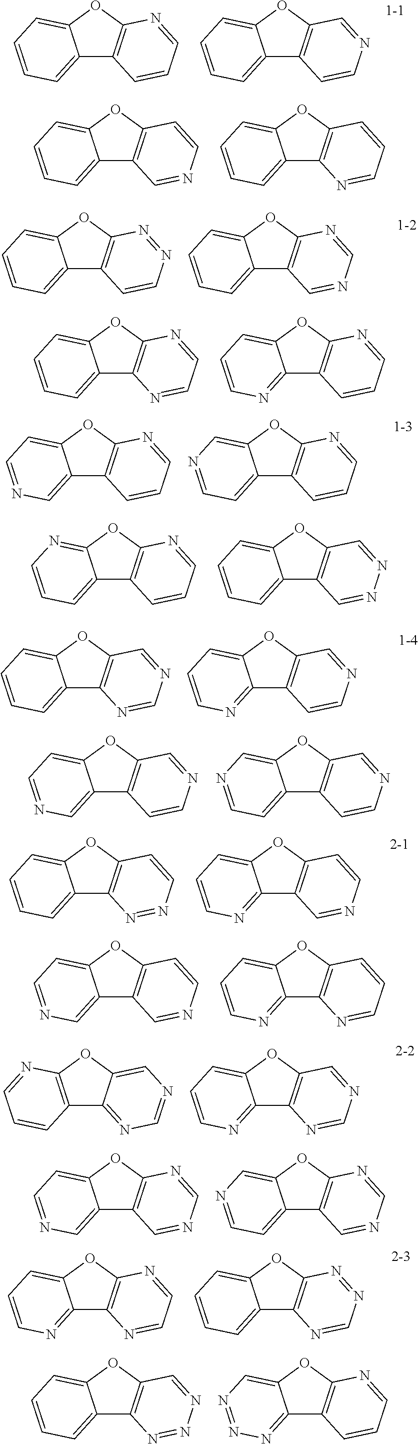

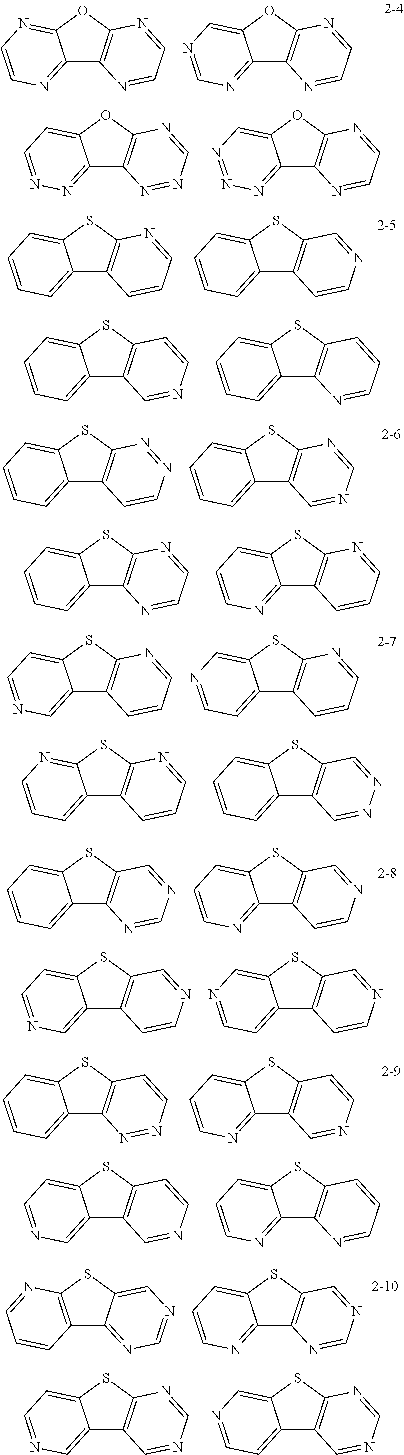

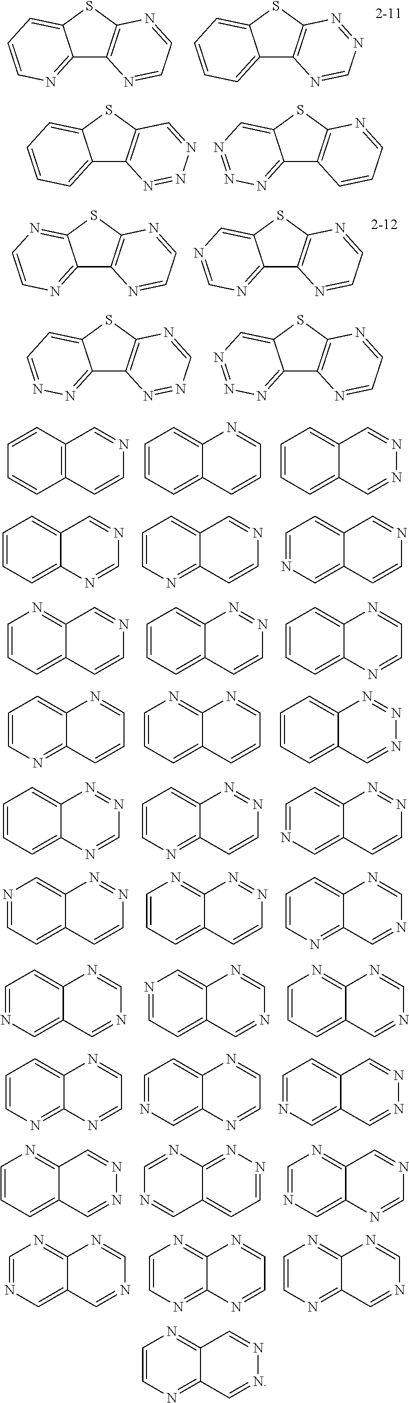

In some embodiments, the above formula III has any one of the following structures:

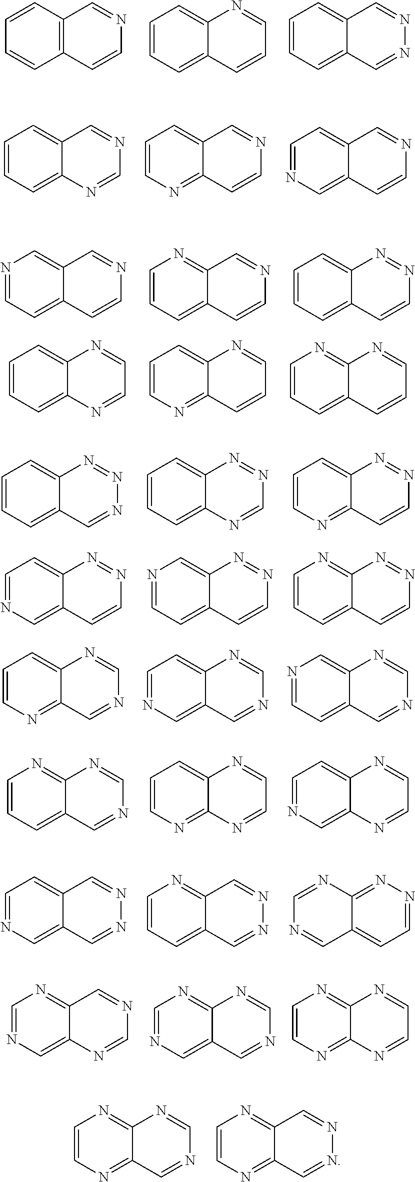

In some embodiments, the above formula III has any one of the following structures:

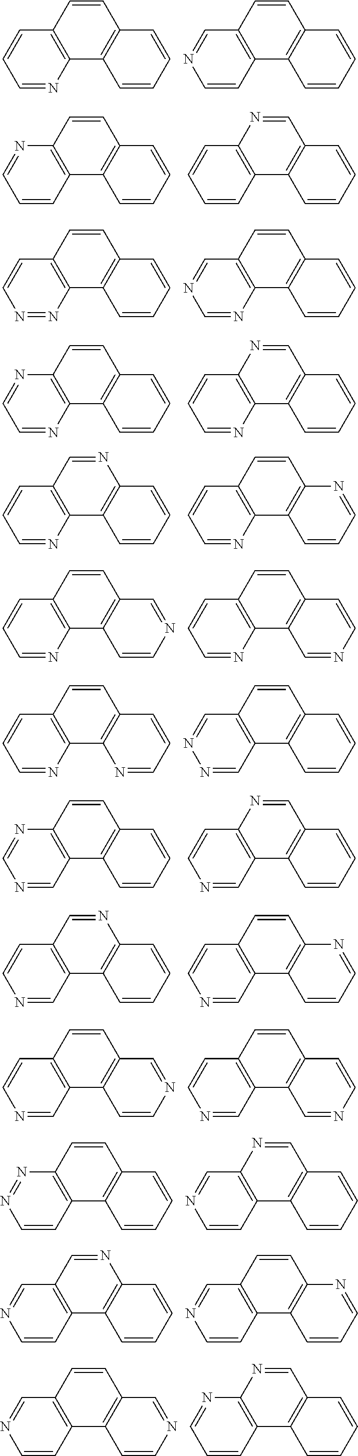

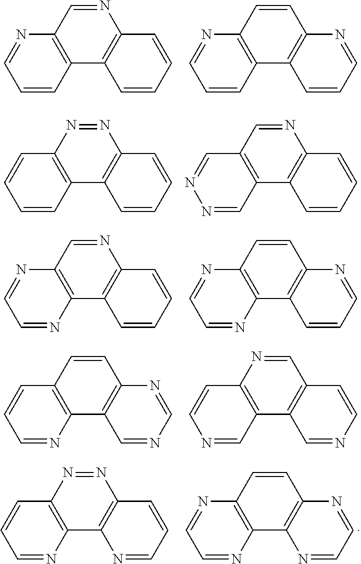

In one embodiment, the Hy₁ and Hy₂ are connected through any C atom in the Hy₁ structure and any C atom in the Hy₂ structure, and one side or both sides adjacent to the connecting C atom in the Hy₁ is an N atom, or one side or both sides adjacent to the connecting C atom in the Hy₂ is an N atom.

In one embodiment, the Hy₁ and Hy₂ are connected through any C atom in the Hy₁ structure and any C atom in the Hy₂ structure, and one side or both sides adjacent to the connecting C atom in the Hy₁ is an N atom, and one side or both sides adjacent to the connecting C atom in the Hy₂ is an N atom.

In some embodiments, the Hy₁ and Hy₂ are connected through the C atom connected with #in the Hy₁ structure and any C atom in the Hy₂ structure, and one side or both sides adjacent to the connecting C atom in the Hy₂ is an N atom, and the Hy₁ is selected from the following structure:

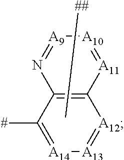

A₉, A₁₀, A₁₁, A₁₂, A₁₃ and A₁₄ are independently selected from the group consisting of N atom or CR₂;

R₂ is selected from the group consisting of hydrogen, deuterium, substituted or unsubstituted C₁-C₆ alkyl or C₂-C₆ alkenyl, and adjacent R₂ can be connected to form a ring;

the Hy₂ has any one of the following structures:

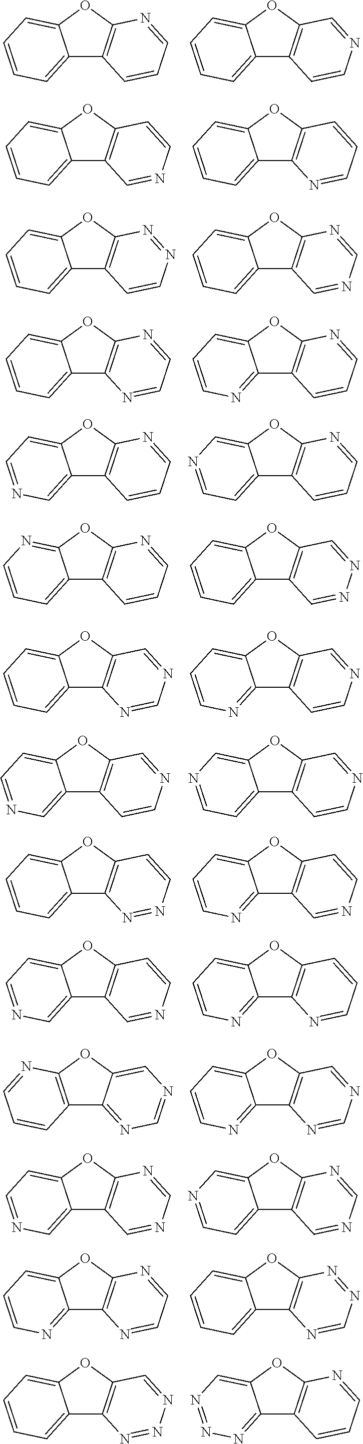

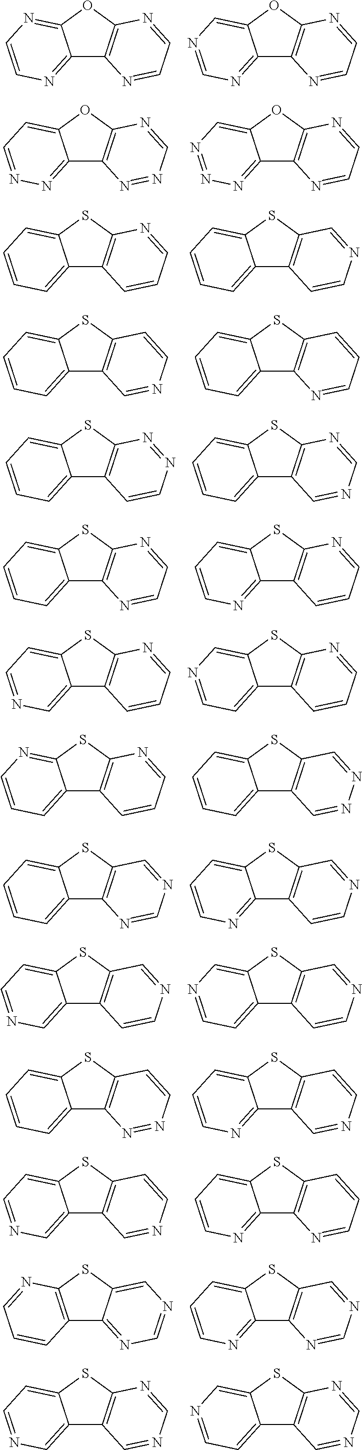

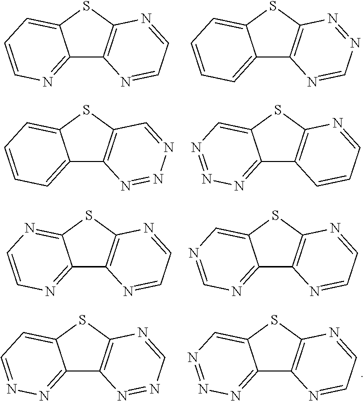

#represents the connecting position of Hy₁ and Hy₁;

##represents the connecting position of Hy₁ and L.

In some embodiments, the Hy₁ and Hy₂ are connected through any C atom in the Hy₁ structure and the C atom connected with #in the Hy₂ structure, and one side or both sides adjacent to the connecting C atom in the Hy₁ is an N atom, and the Hy₂ is selected from the following structure:

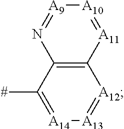

A₉, A₁₀, A₁₁, A₁₂, A₁₃ and A₁₄ are independently selected from the group consisting of N atom or CR₂;

R₂ is selected from the group consisting of hydrogen, deuterium, substituted or unsubstituted C₁-C₆ alkyl or C₂-C₆ alkenyl, and adjacent R₂ can be connected to form a ring;

the Hy₁ has any one of the following structures:

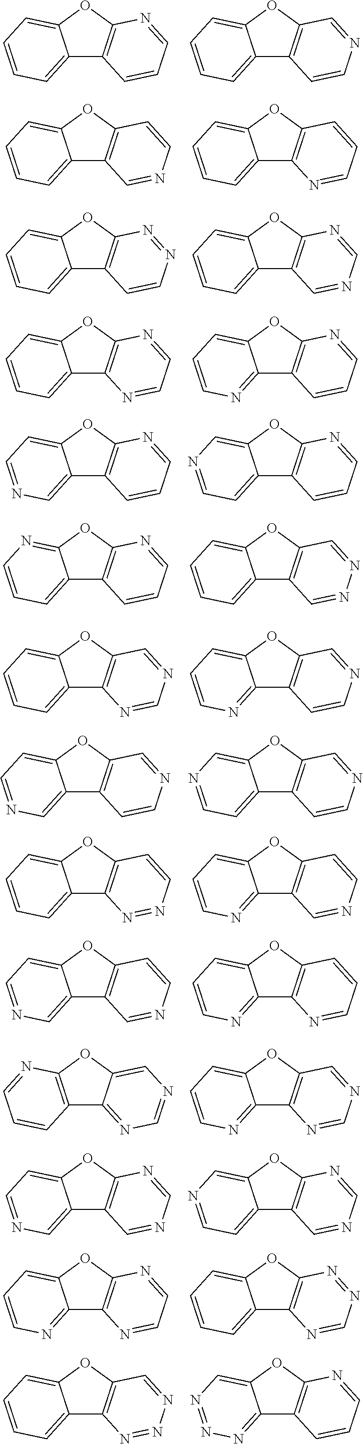

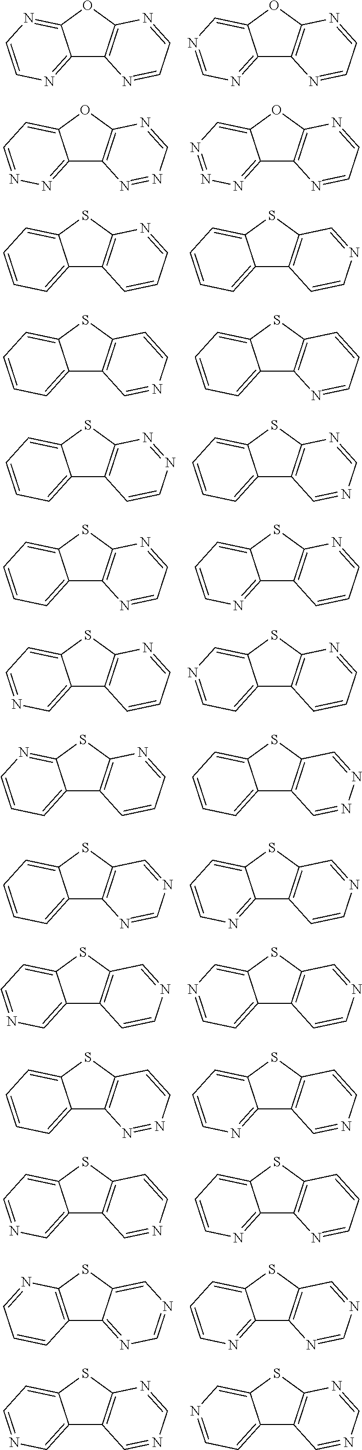

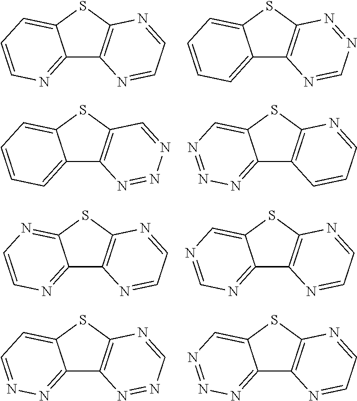

#represents the connecting position of Hy₁ and Hy₁.

In some embodiments, Hy₁-Hy₂ has any one of the following structures:

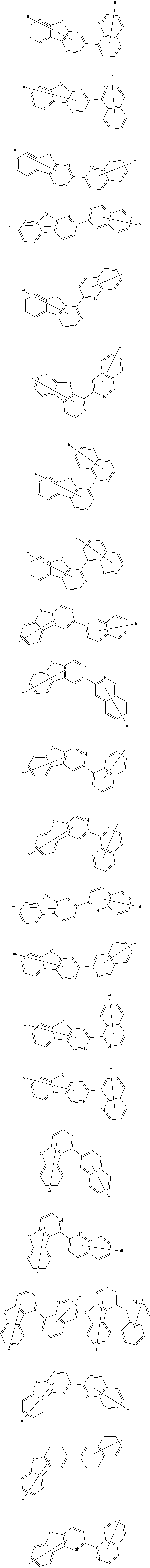

[chemical structure]

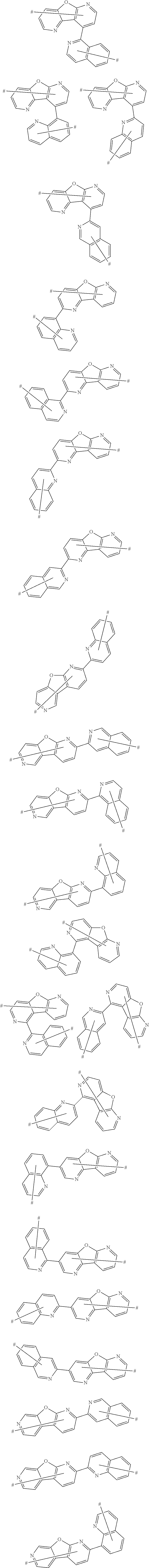

[chemical structure]

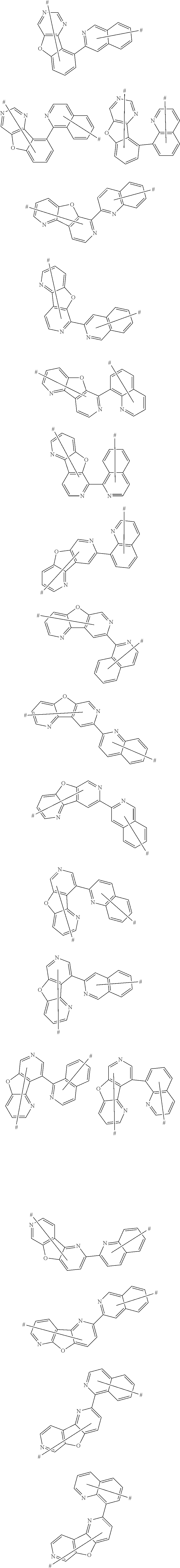

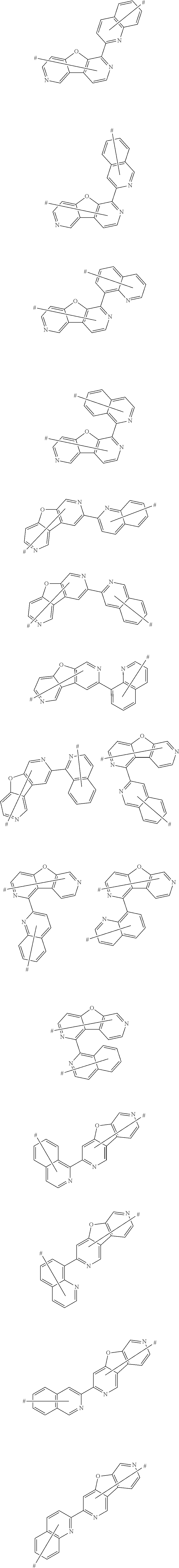

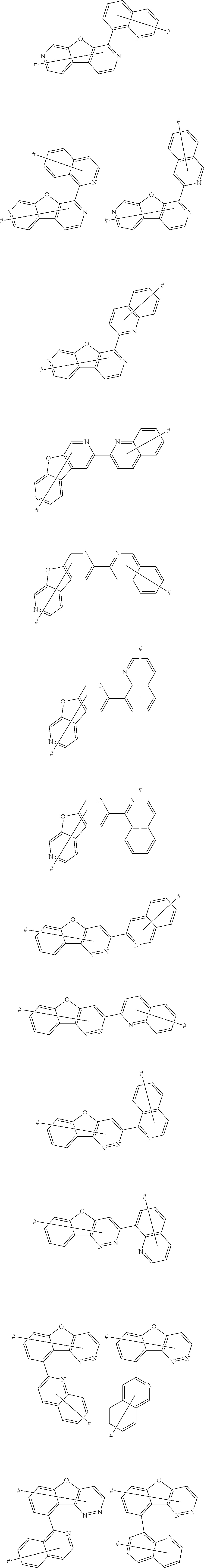

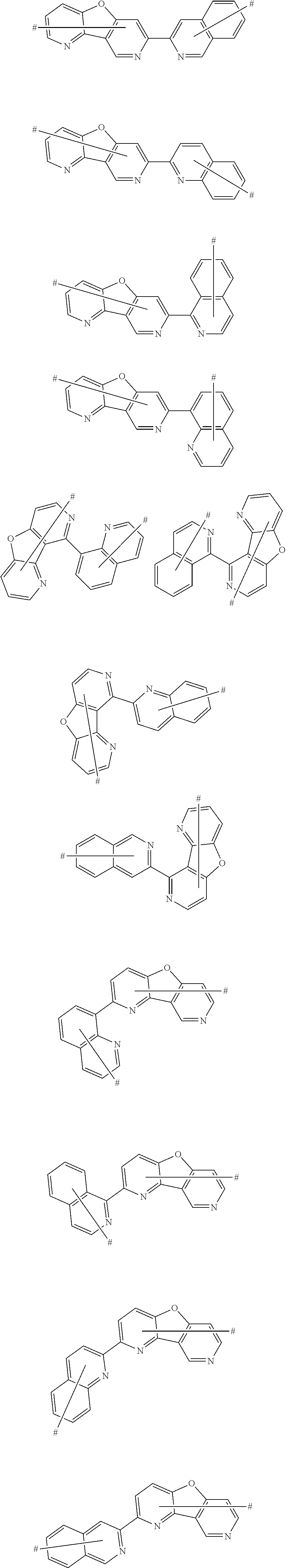

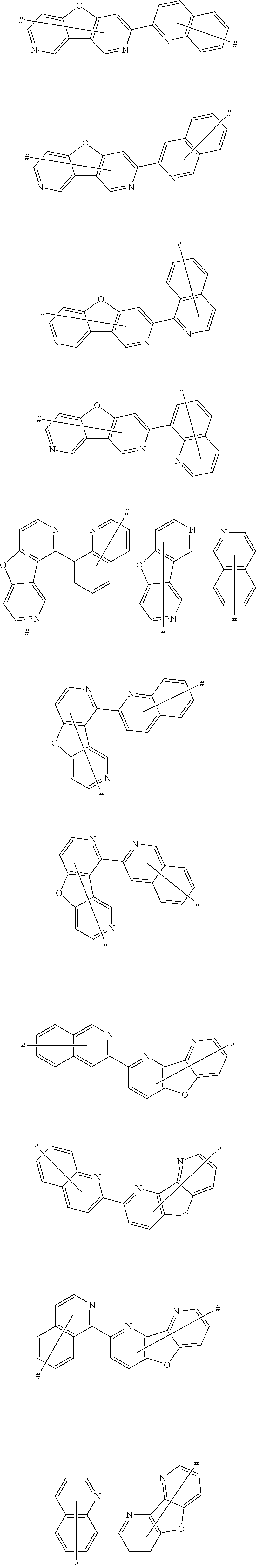

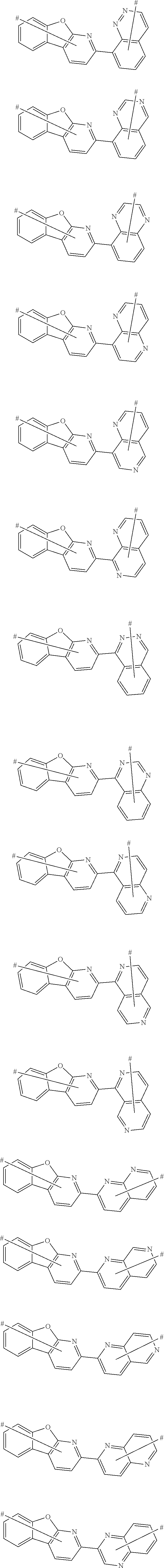

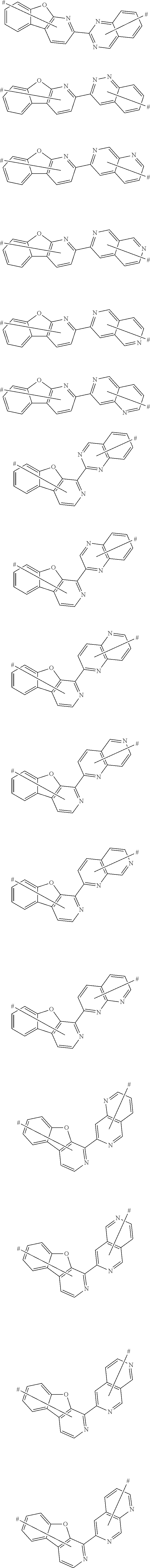

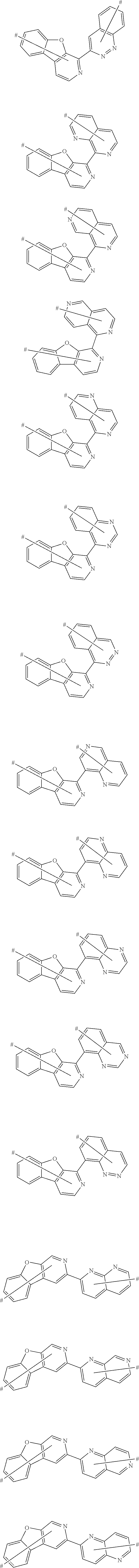

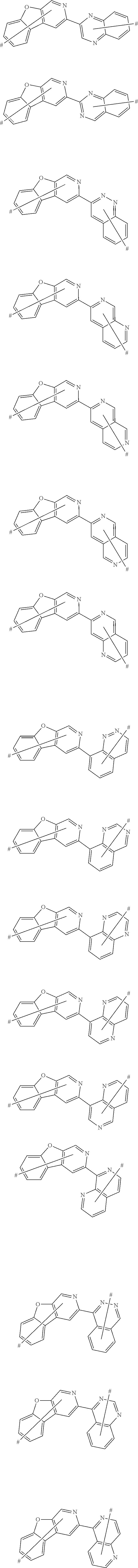

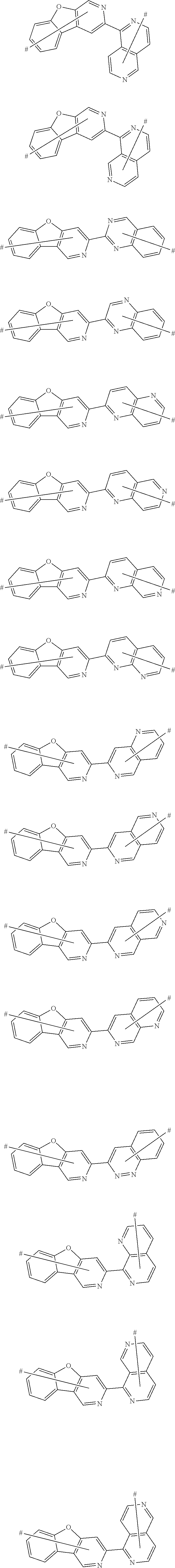

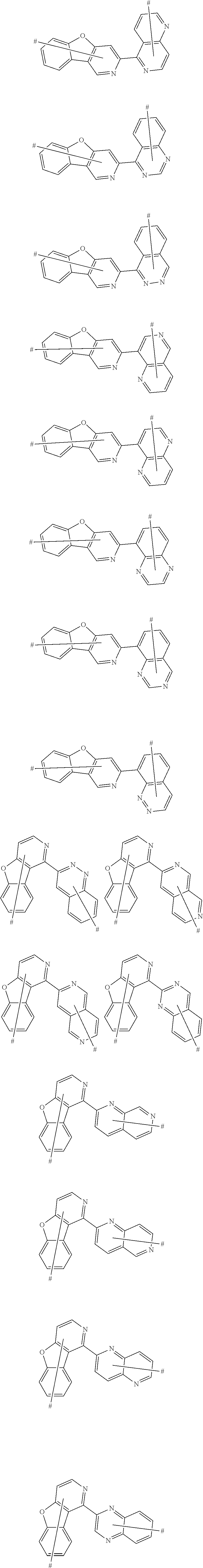

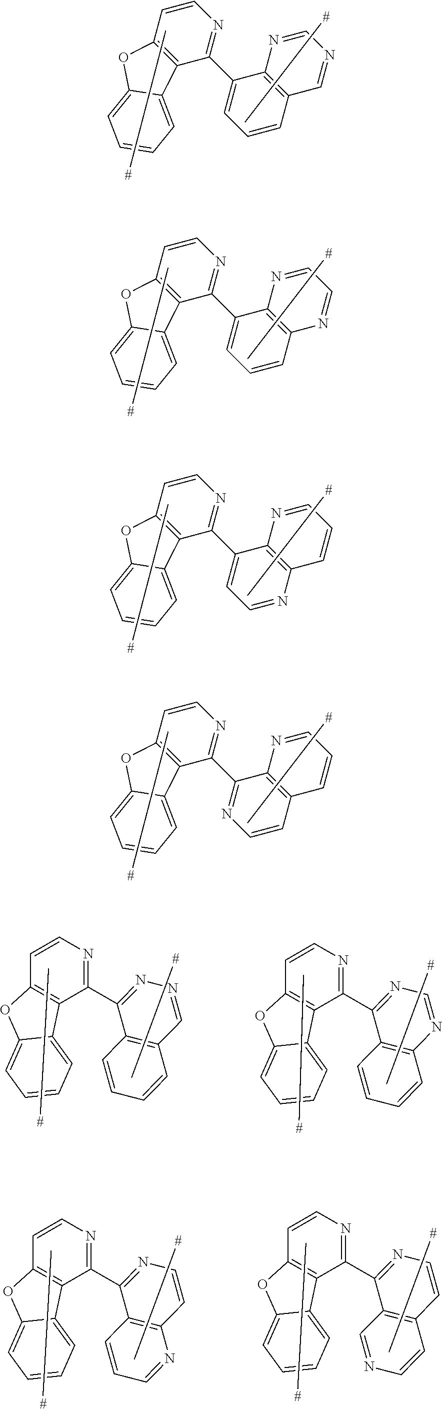

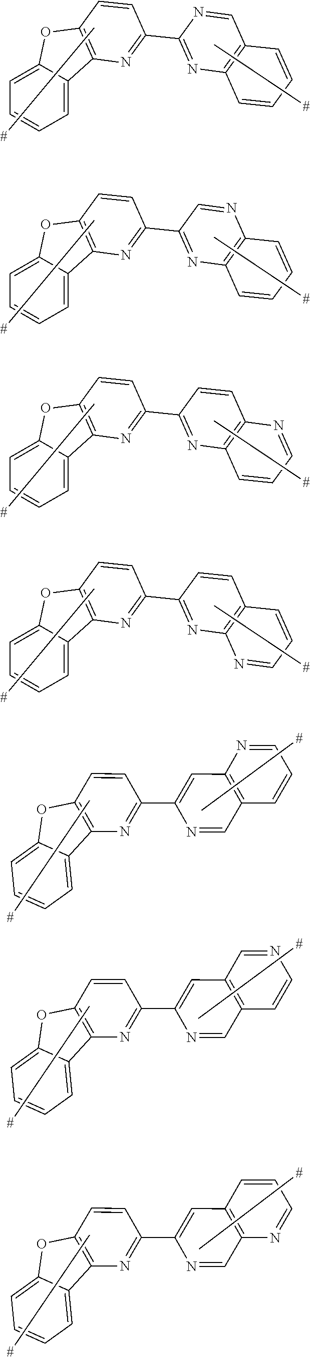

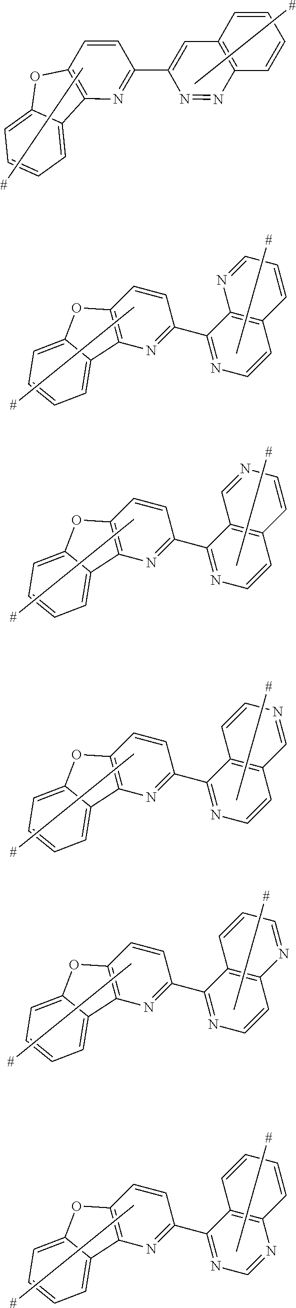

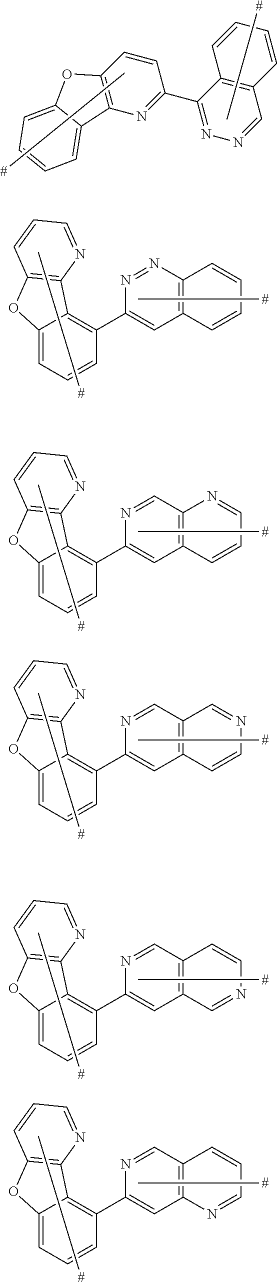

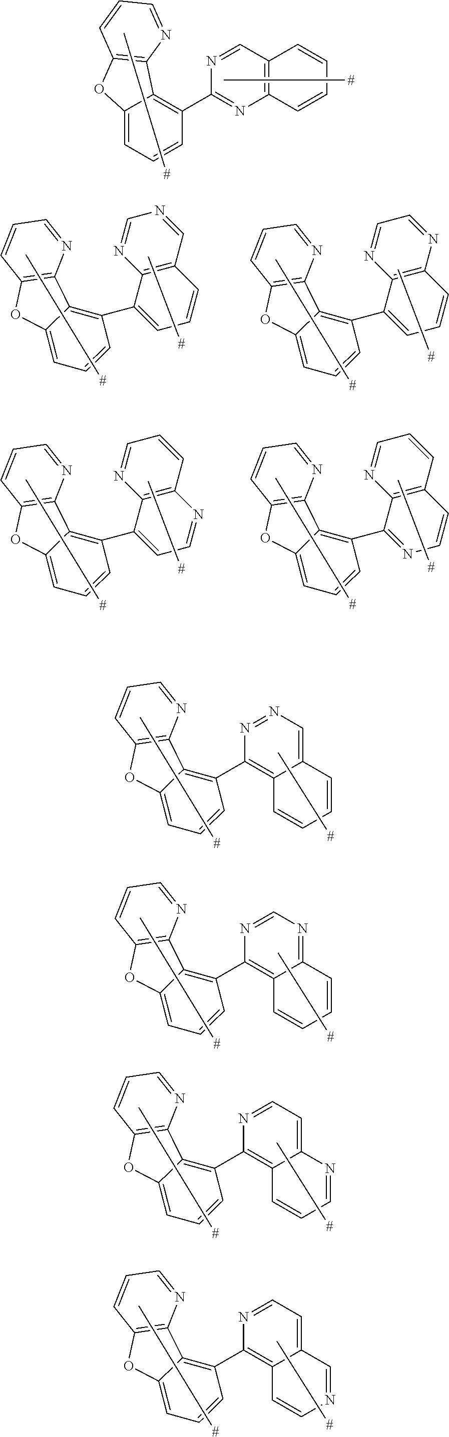

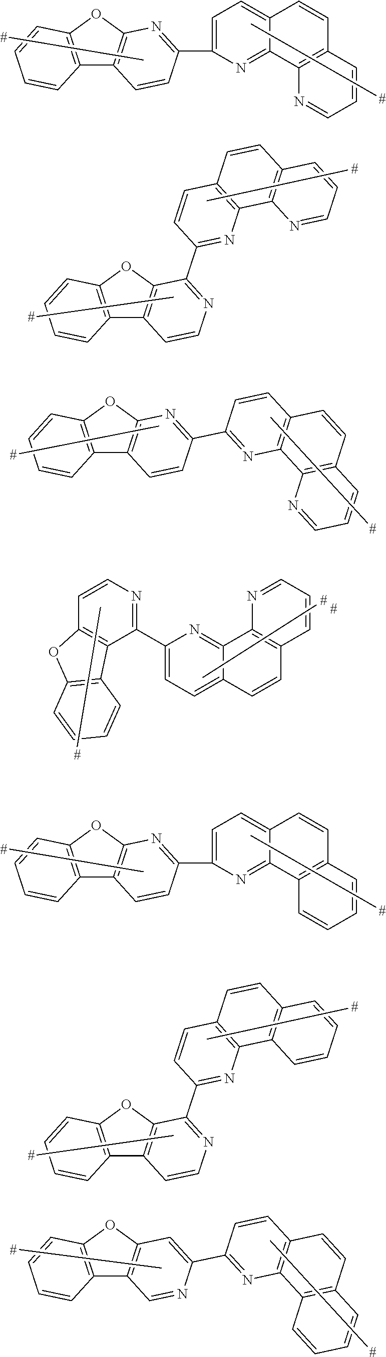

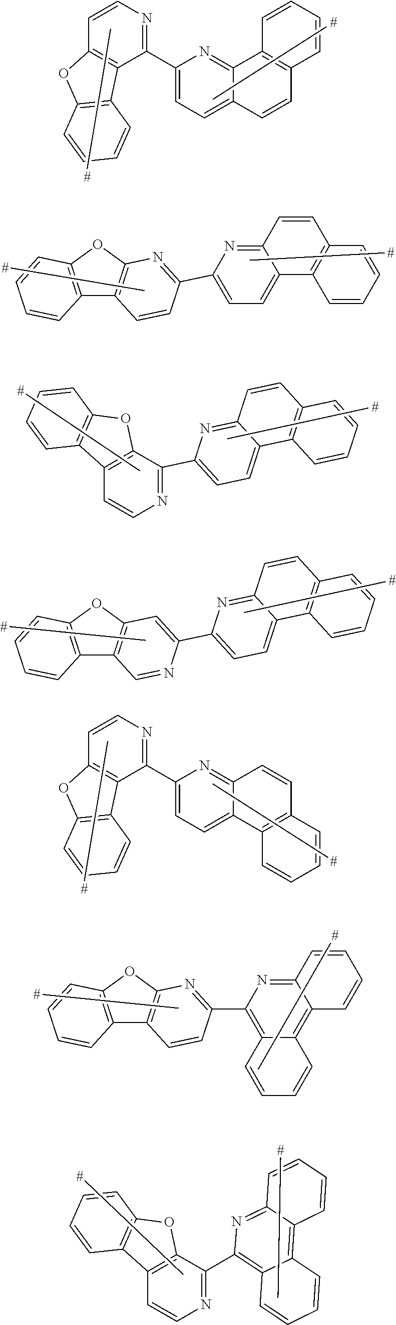

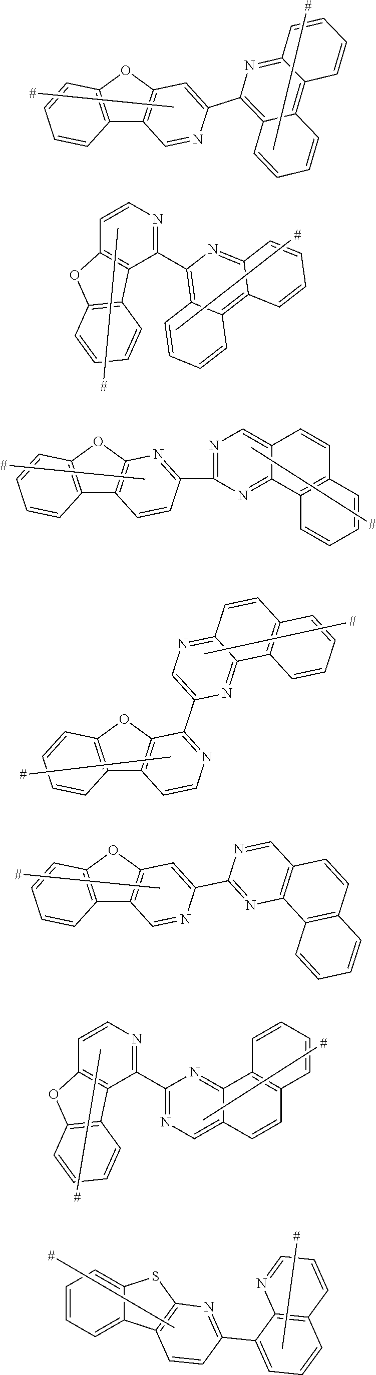

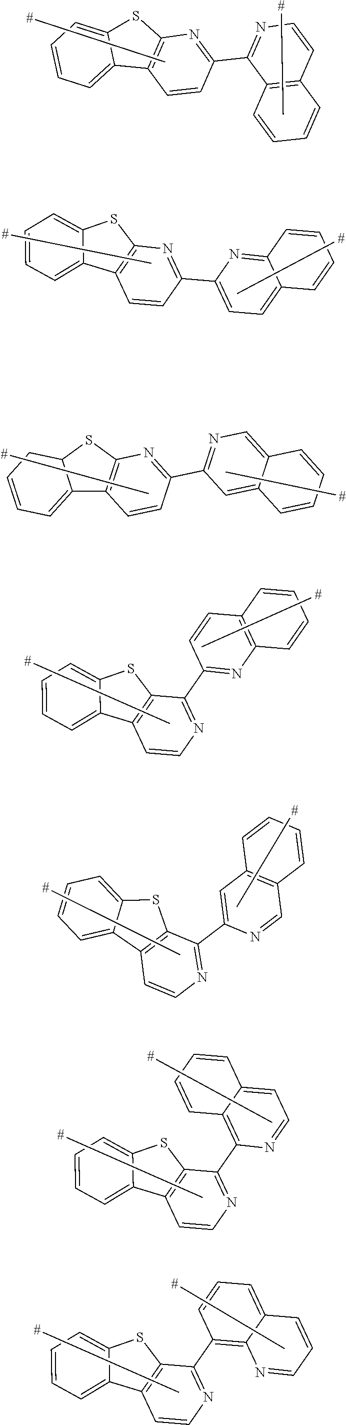

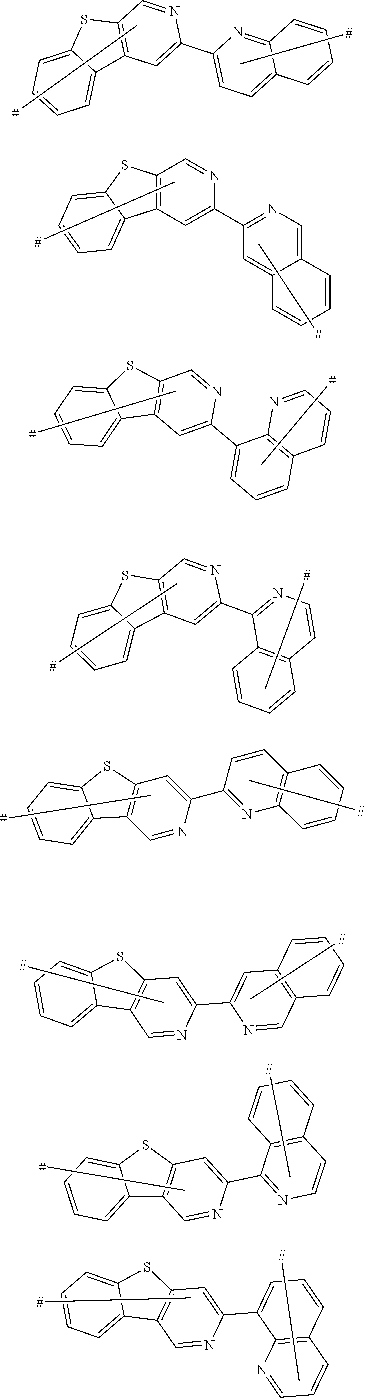

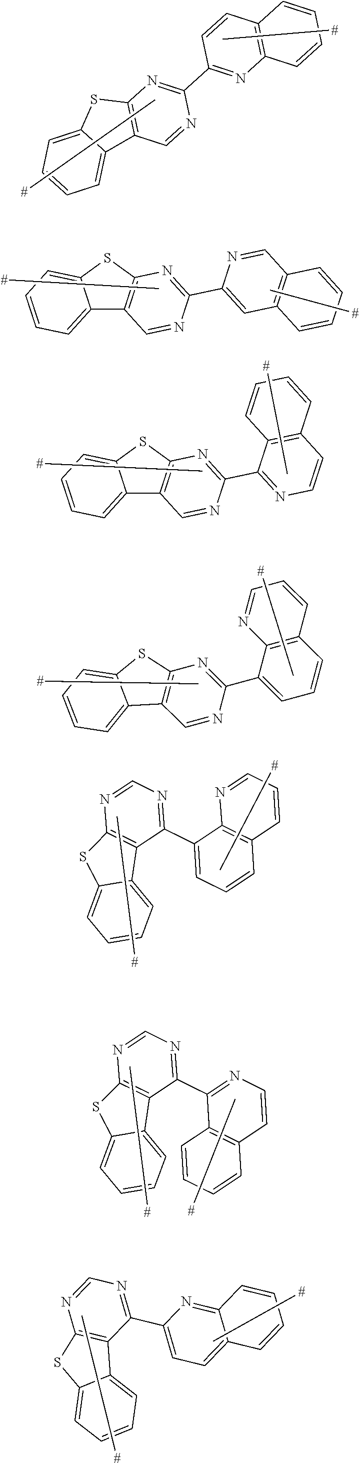

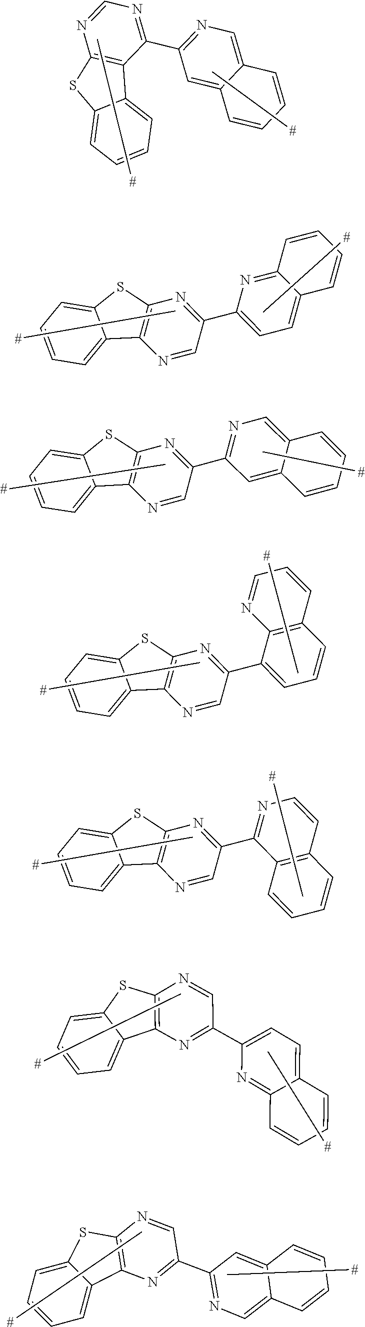

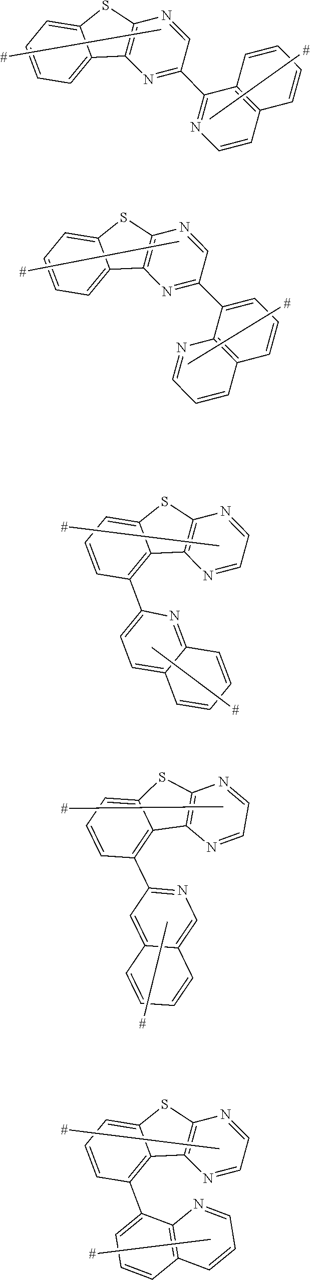

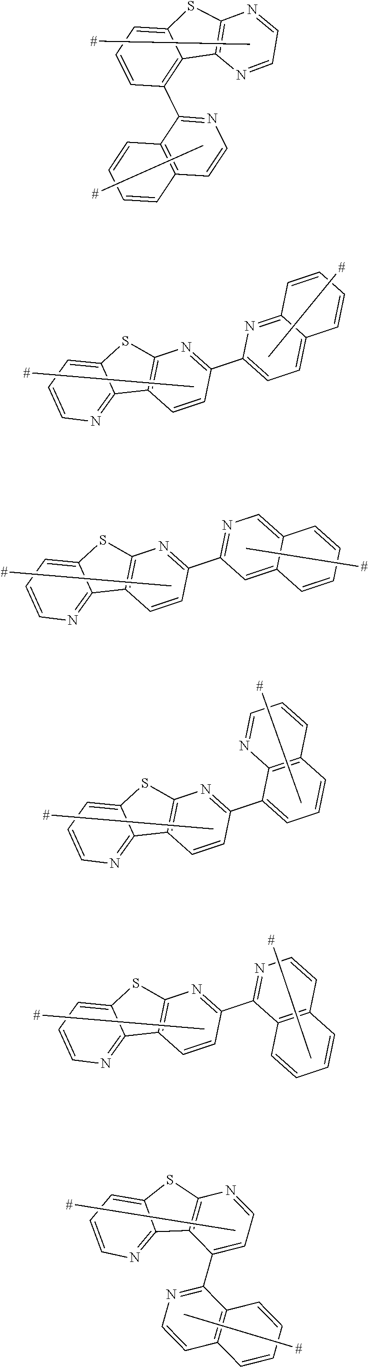

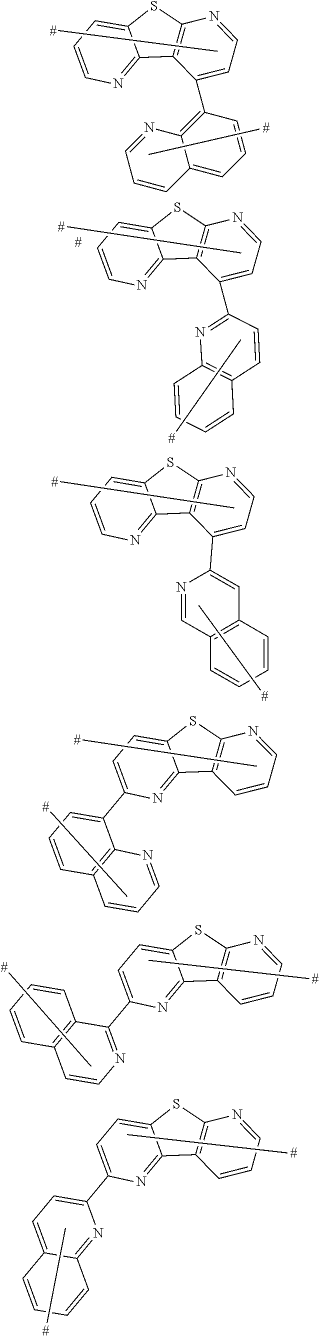

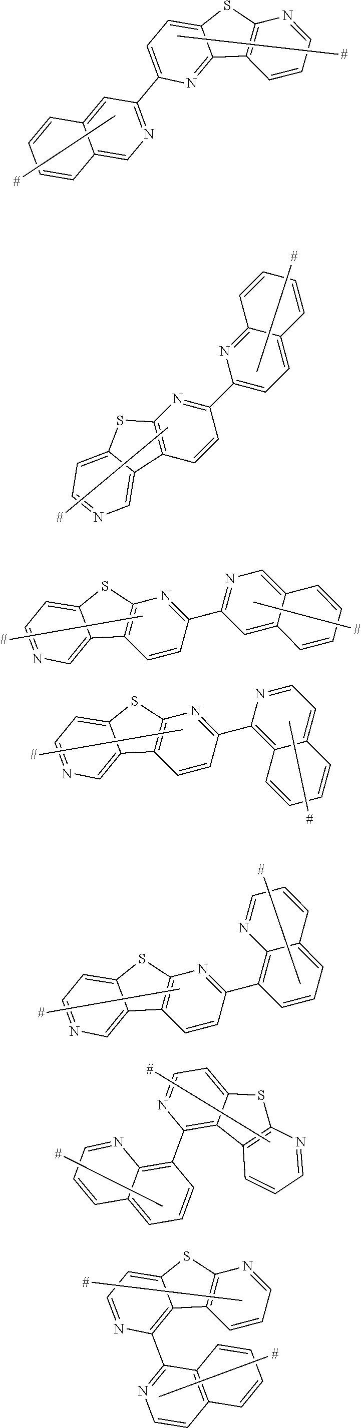

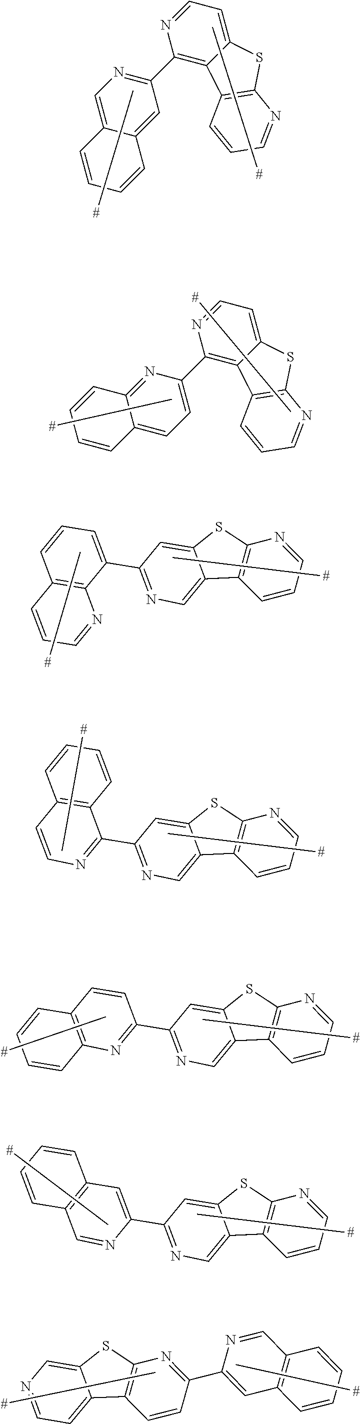

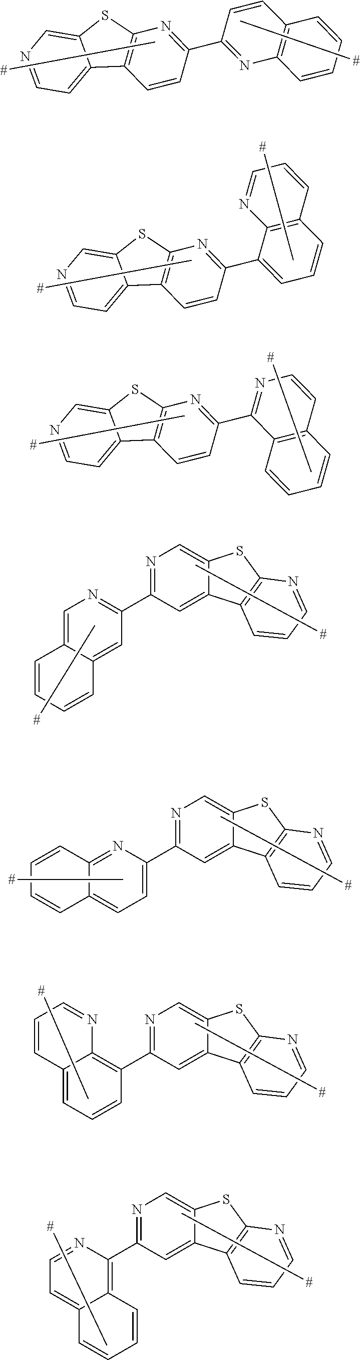

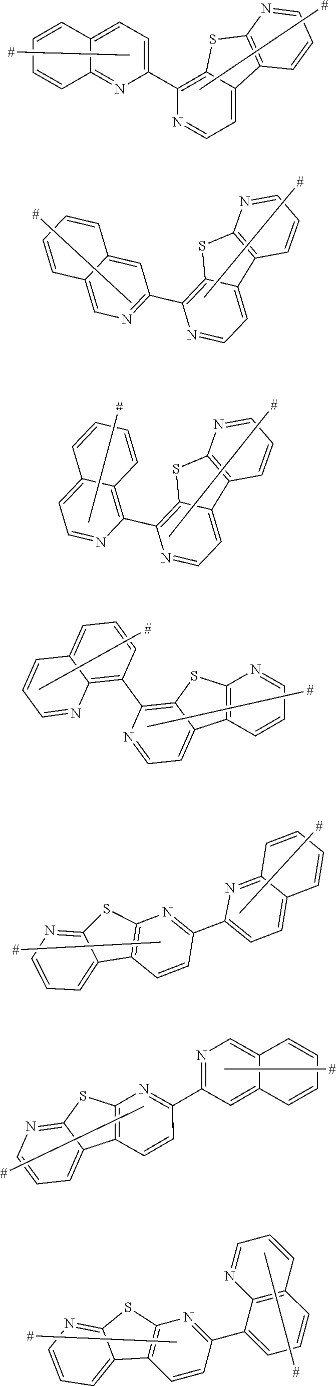

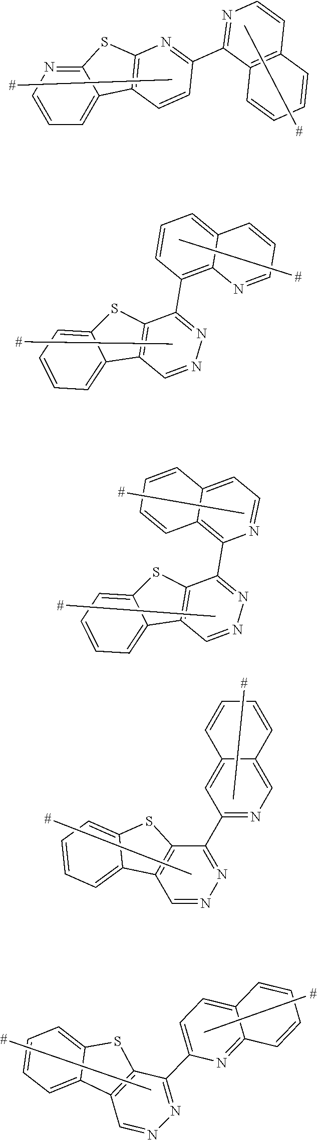

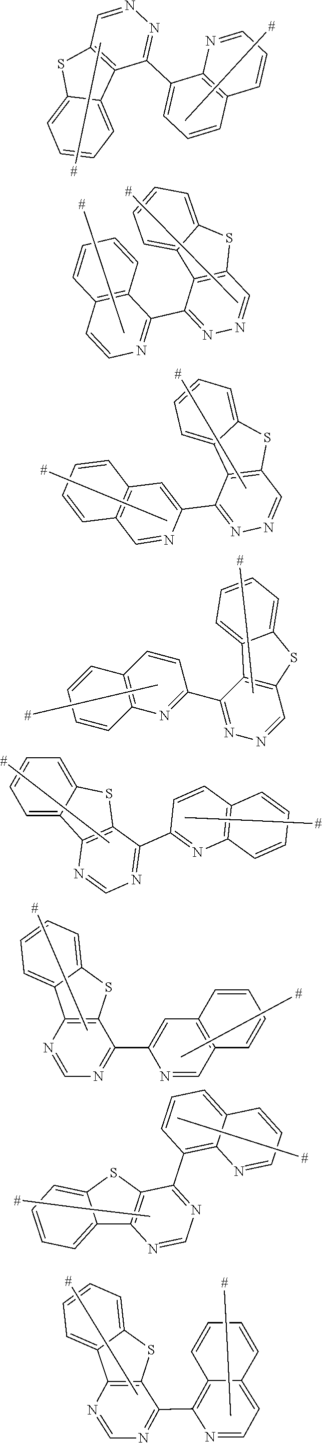

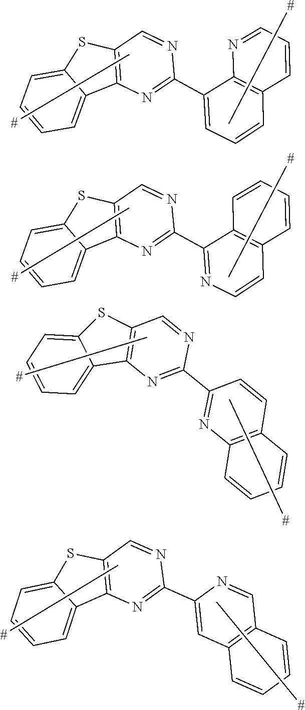

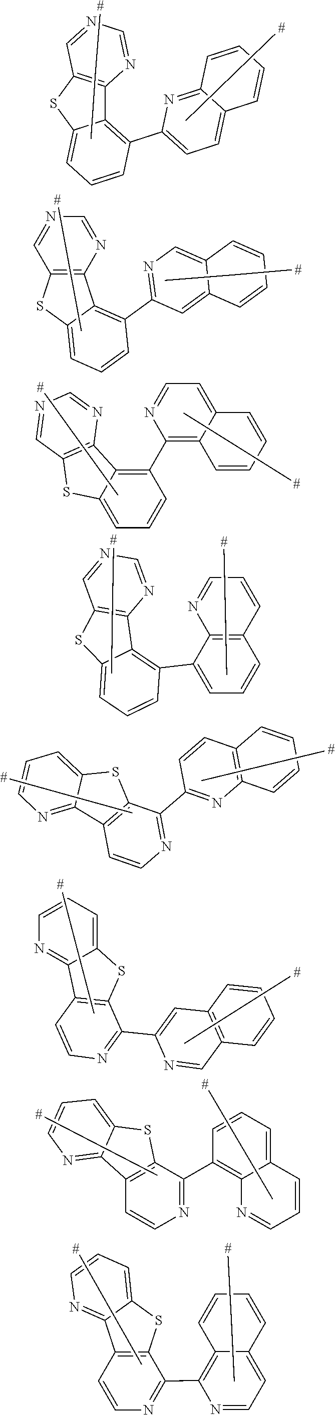

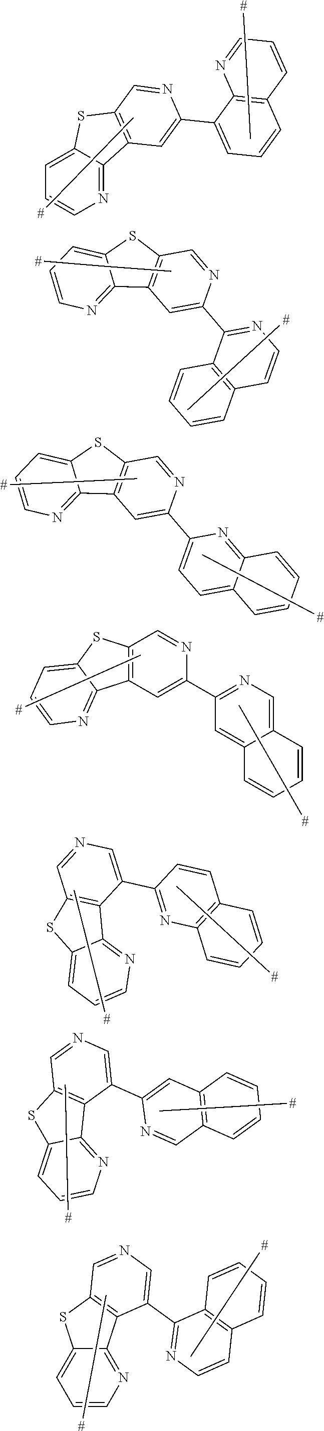

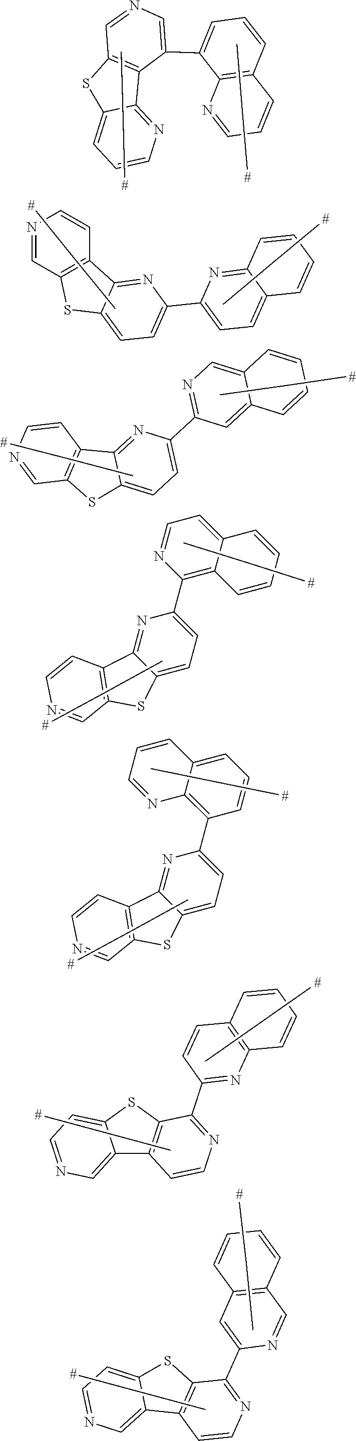

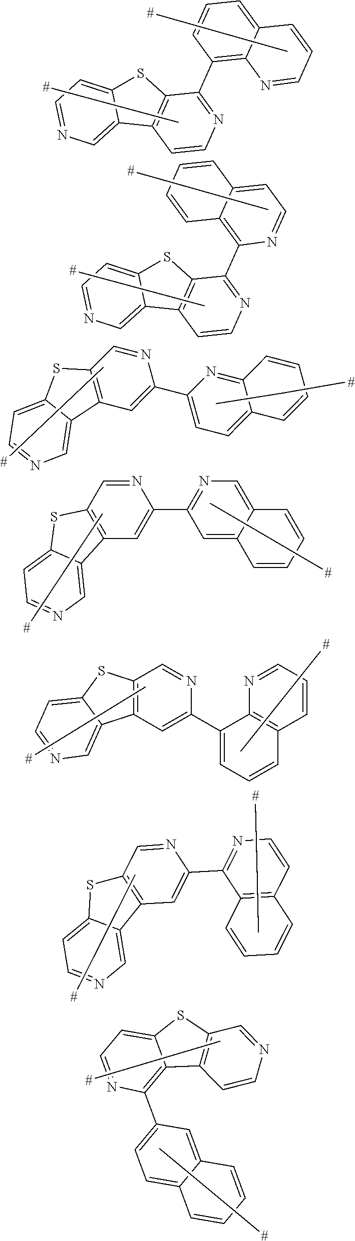

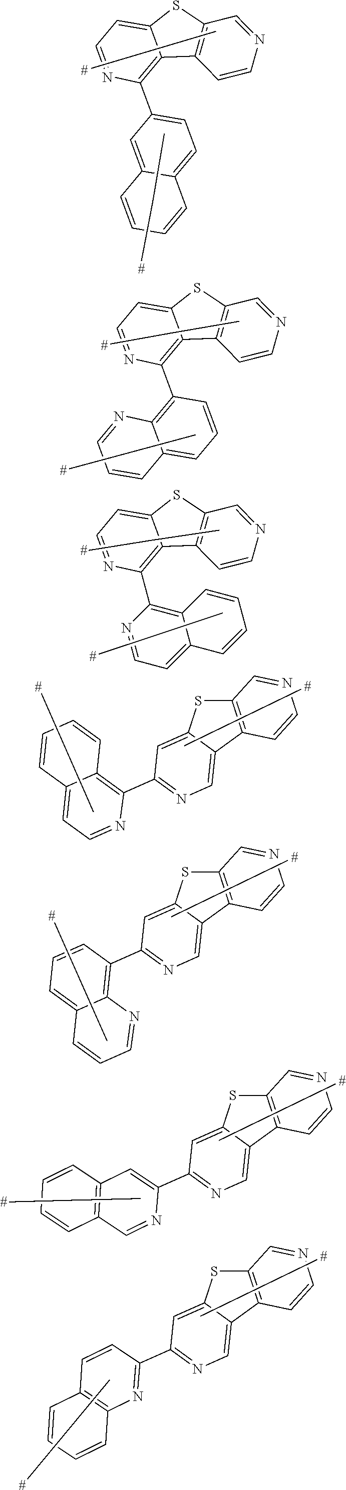

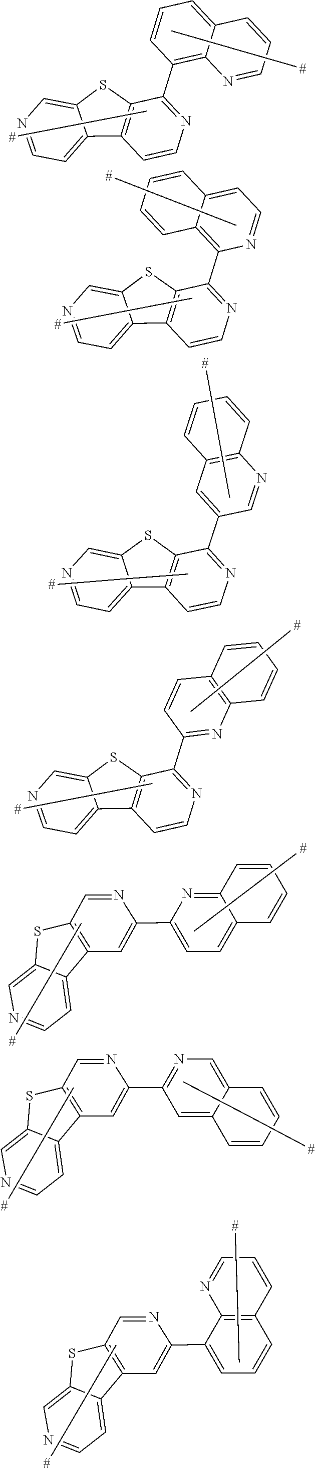

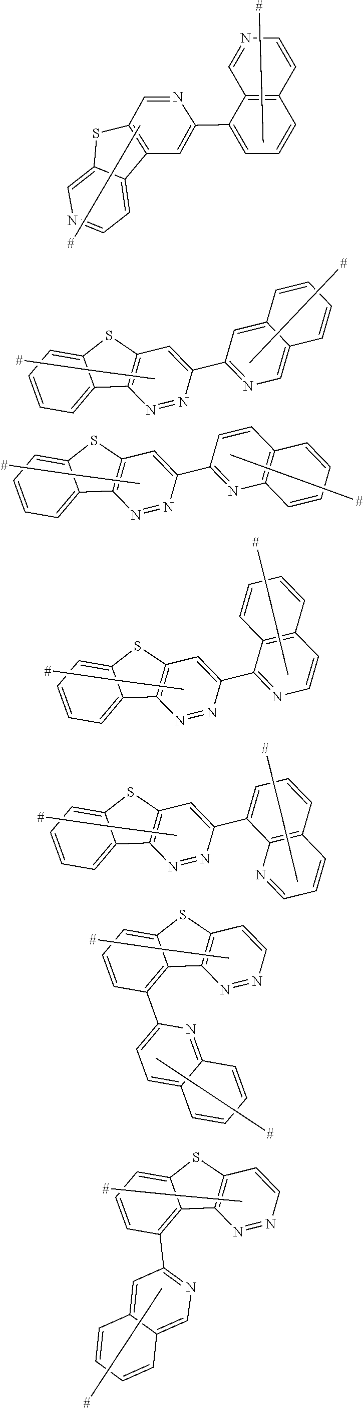

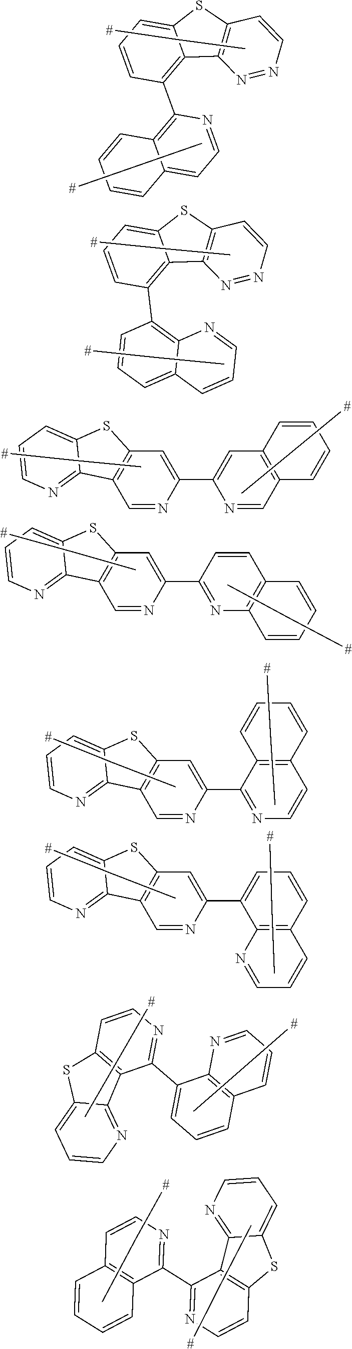

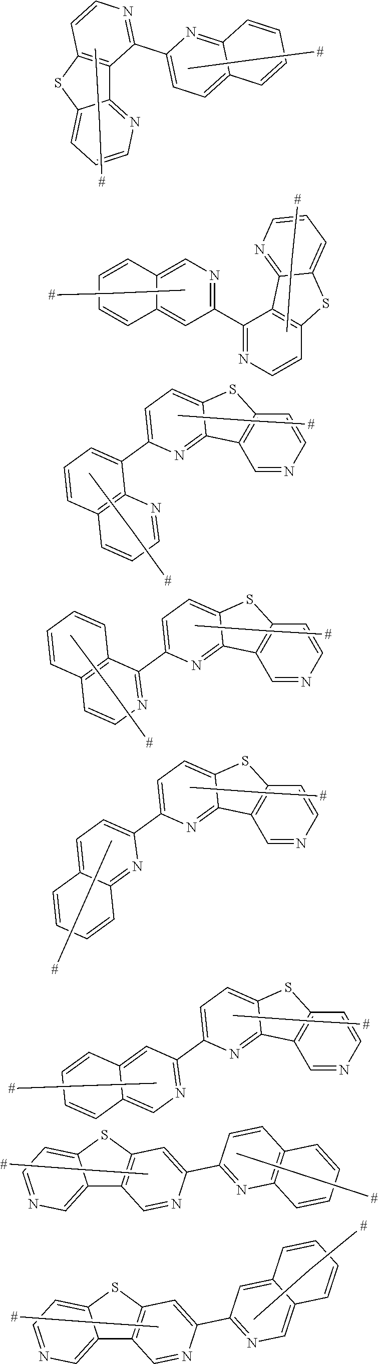

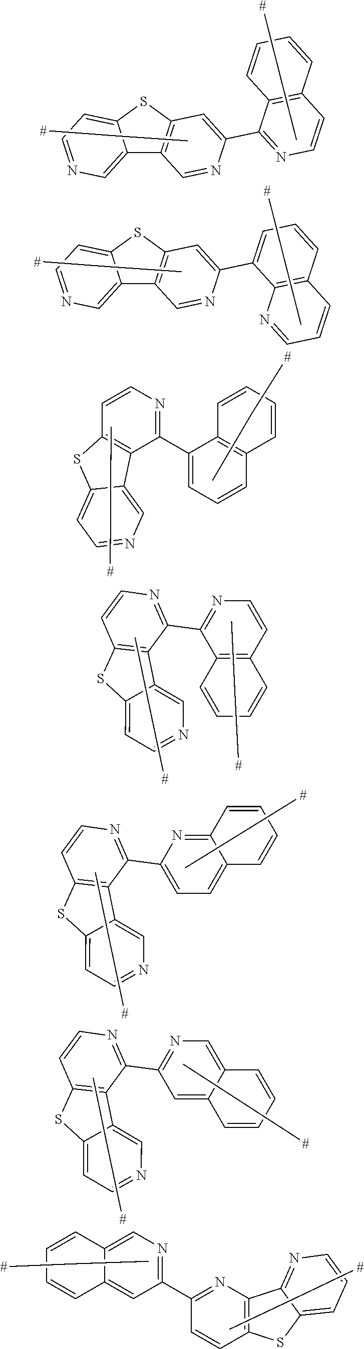

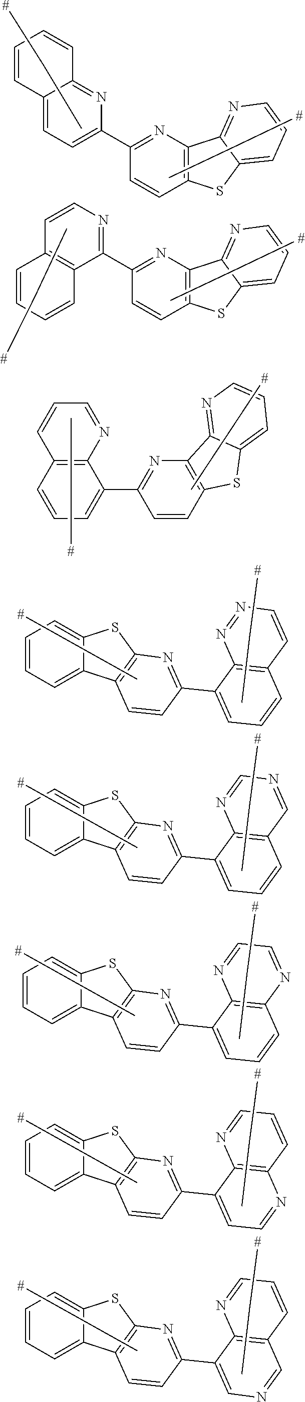

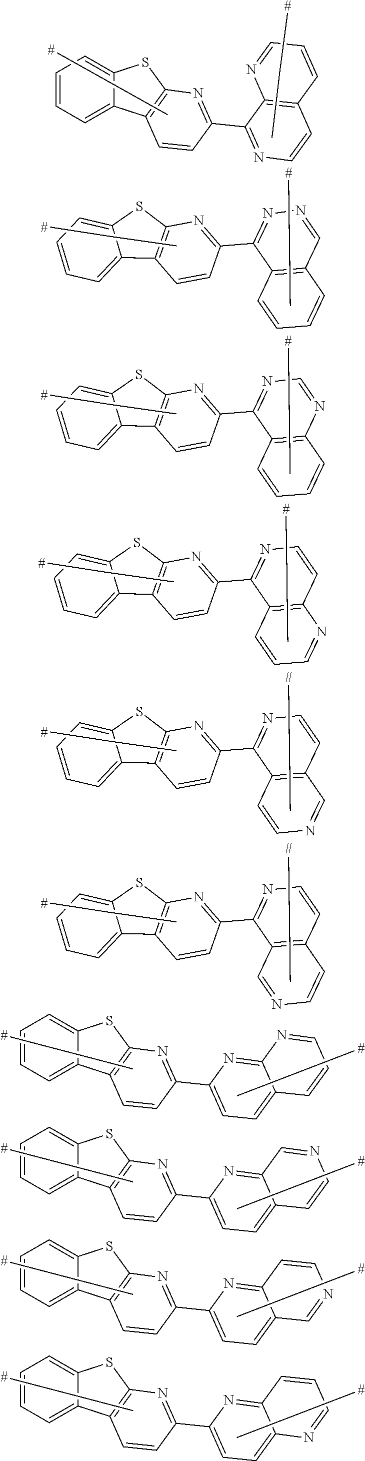

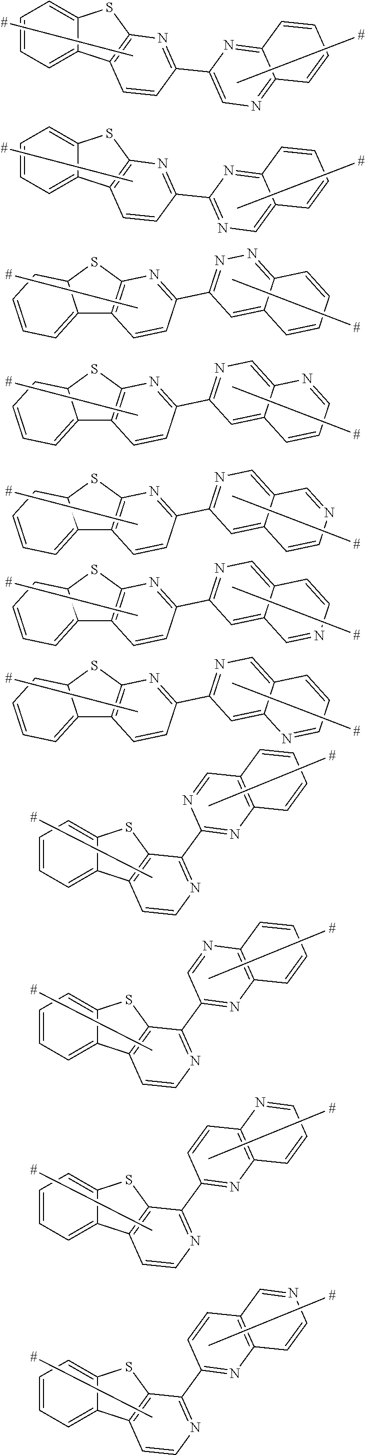

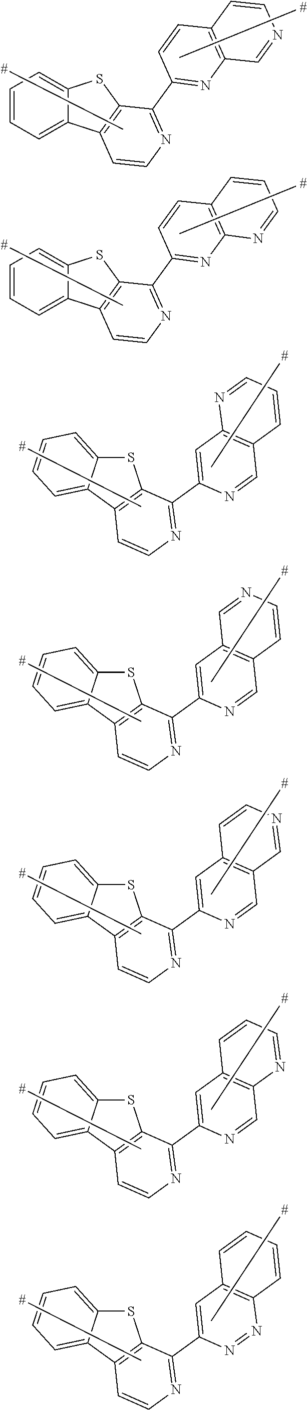

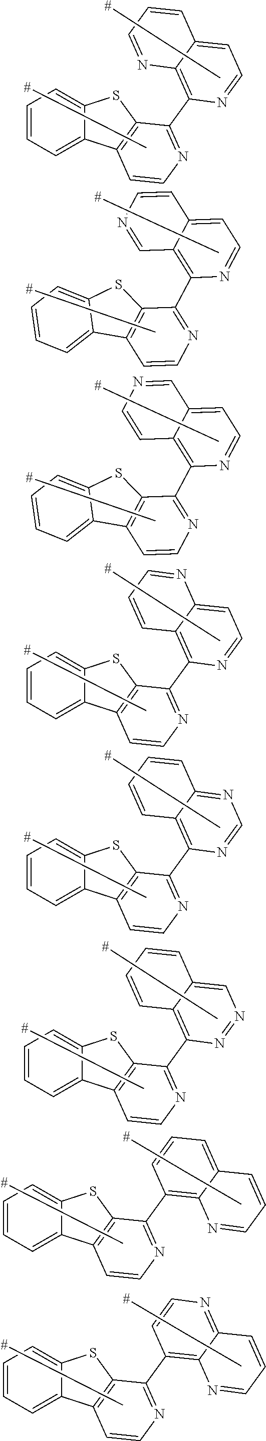

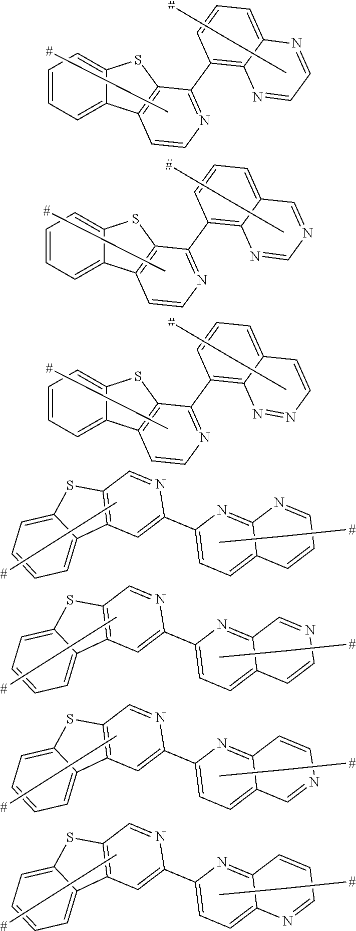

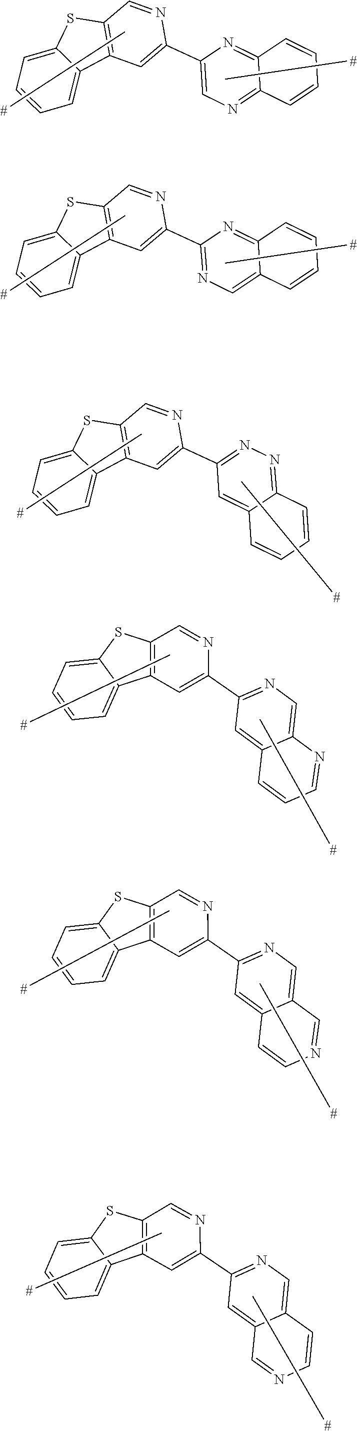

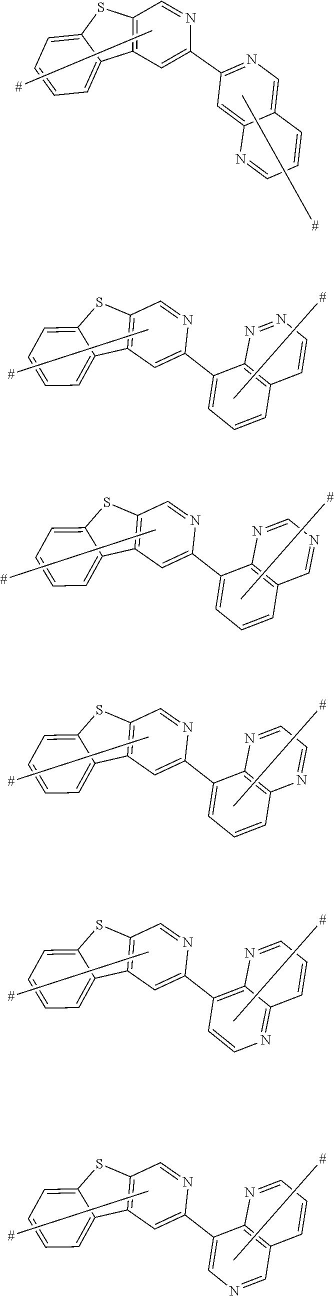

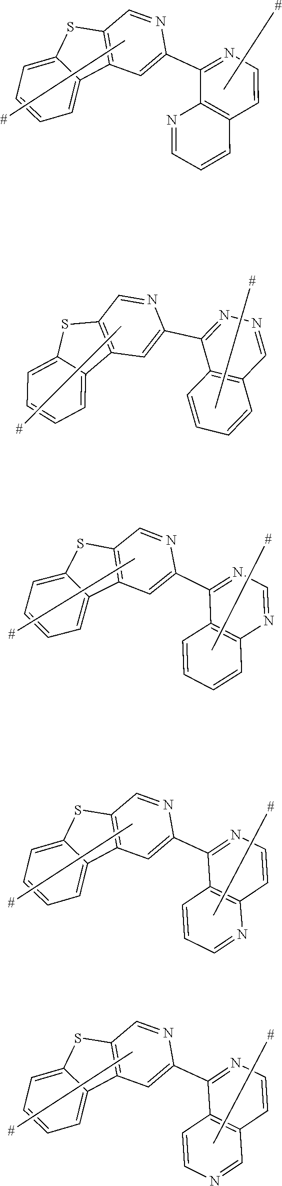

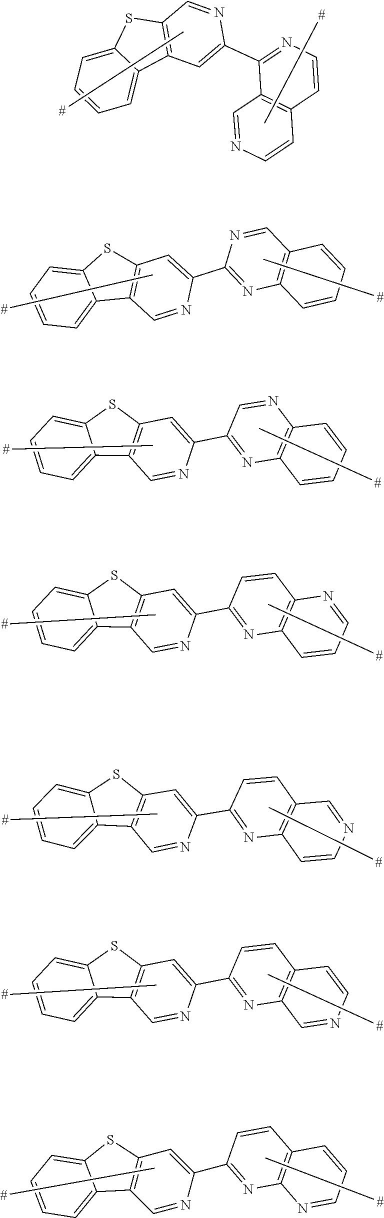

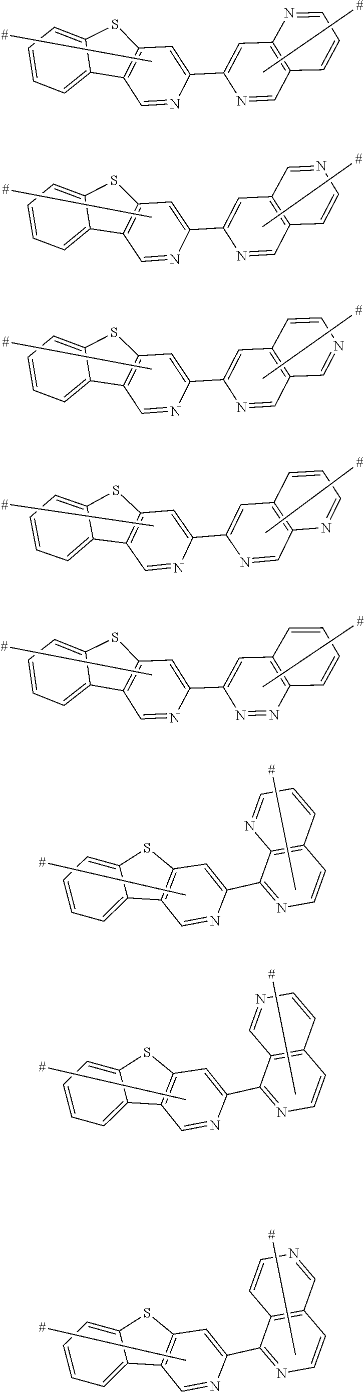

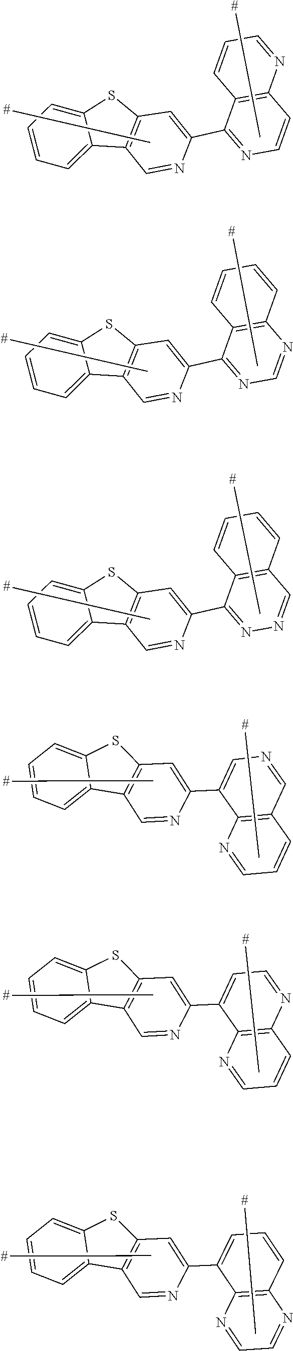

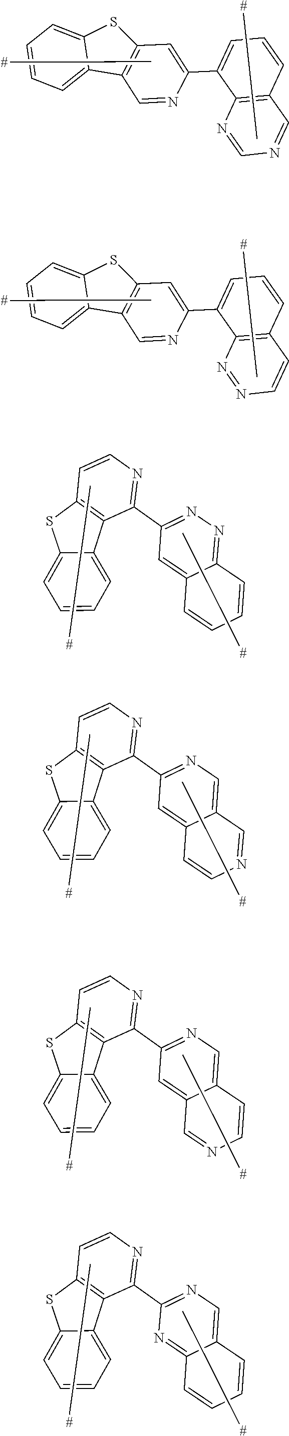

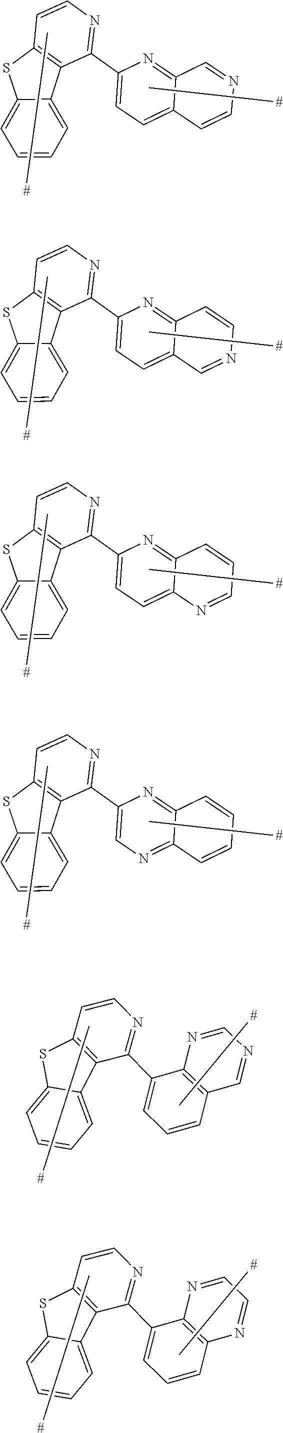

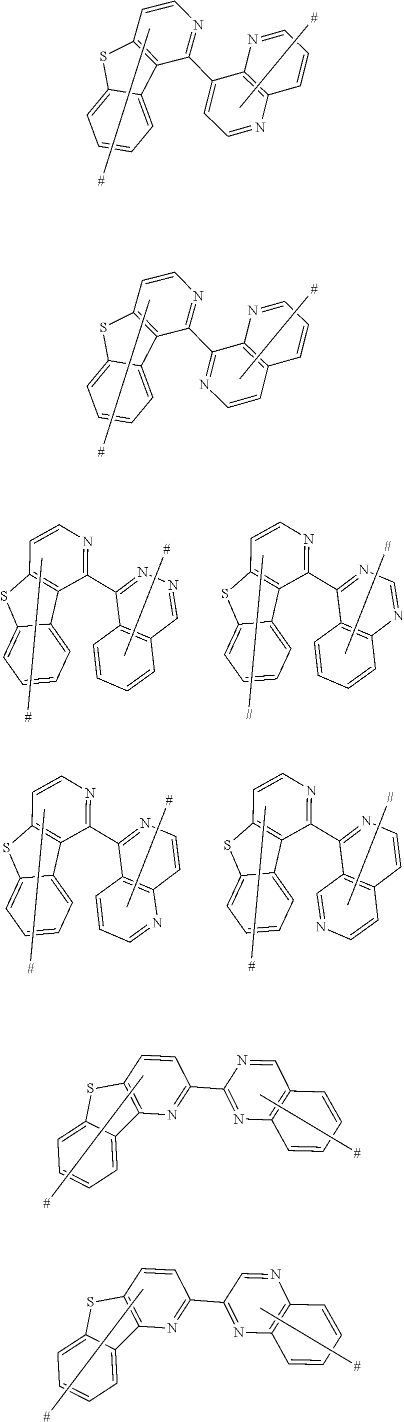

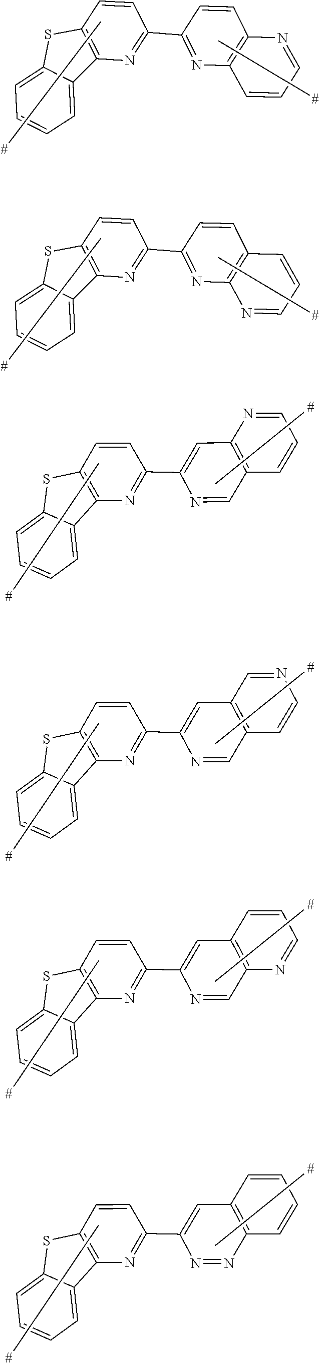

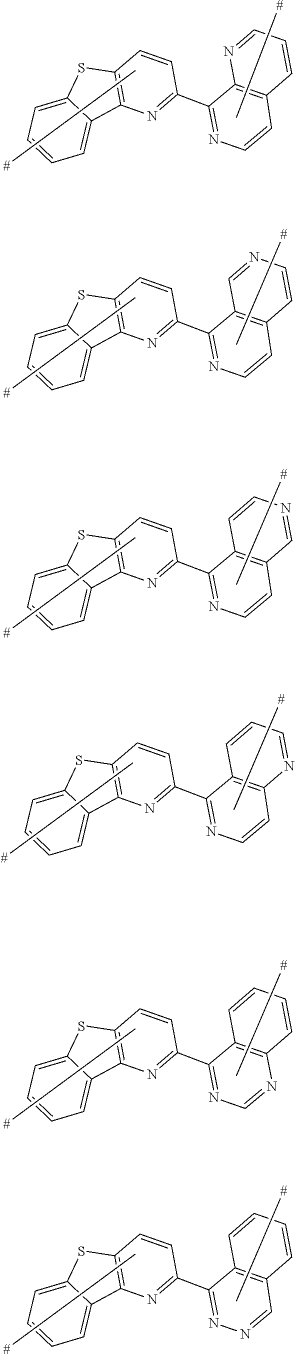

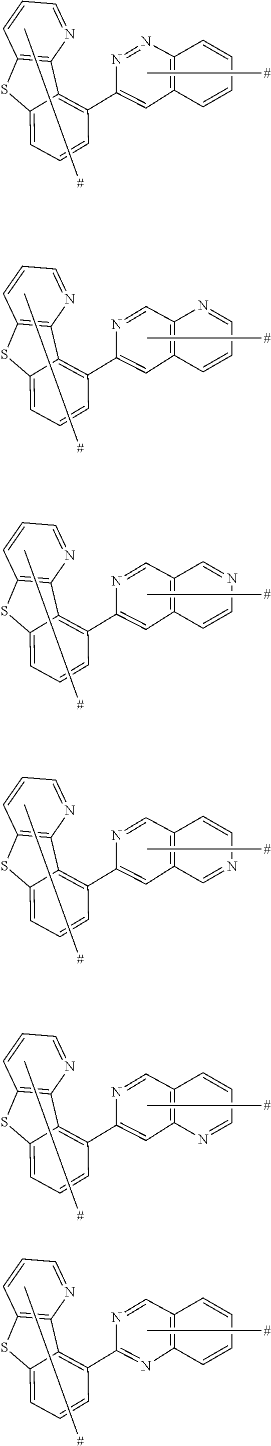

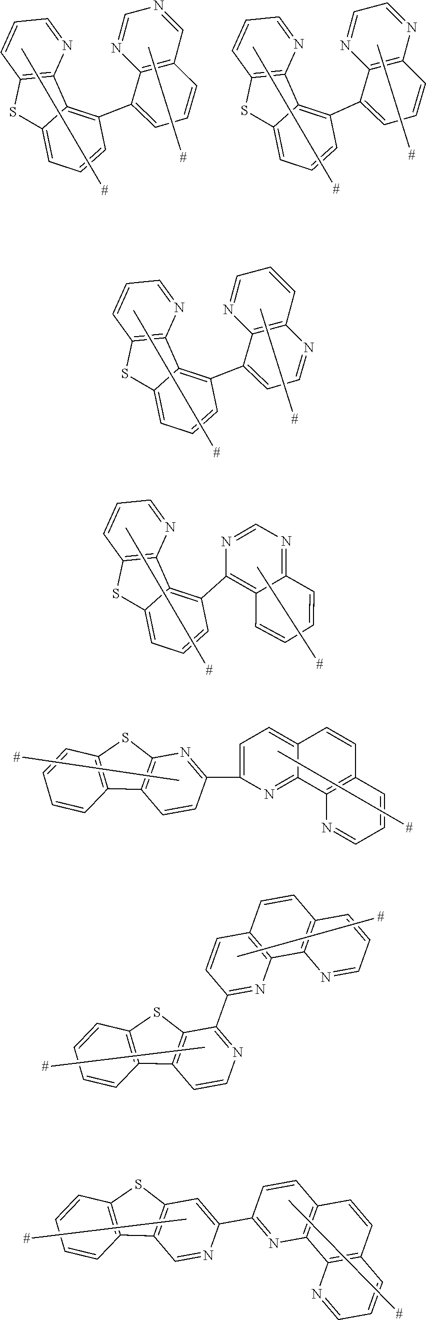

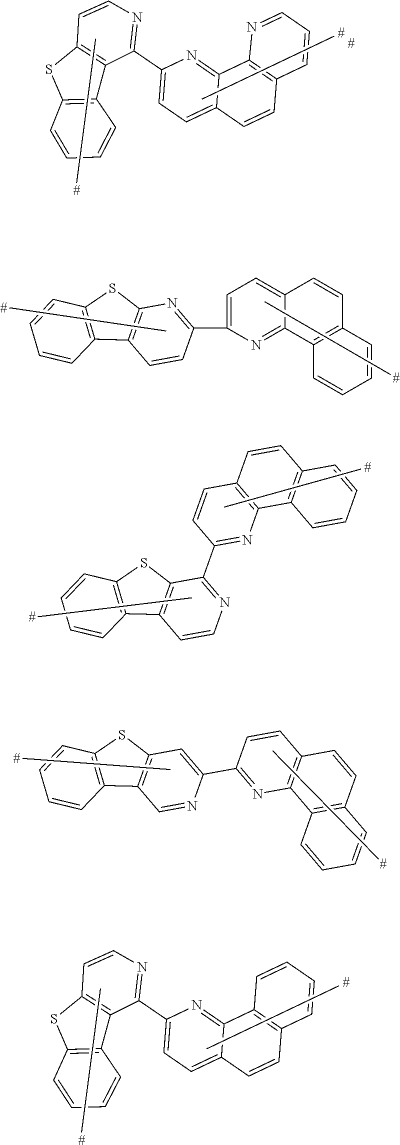

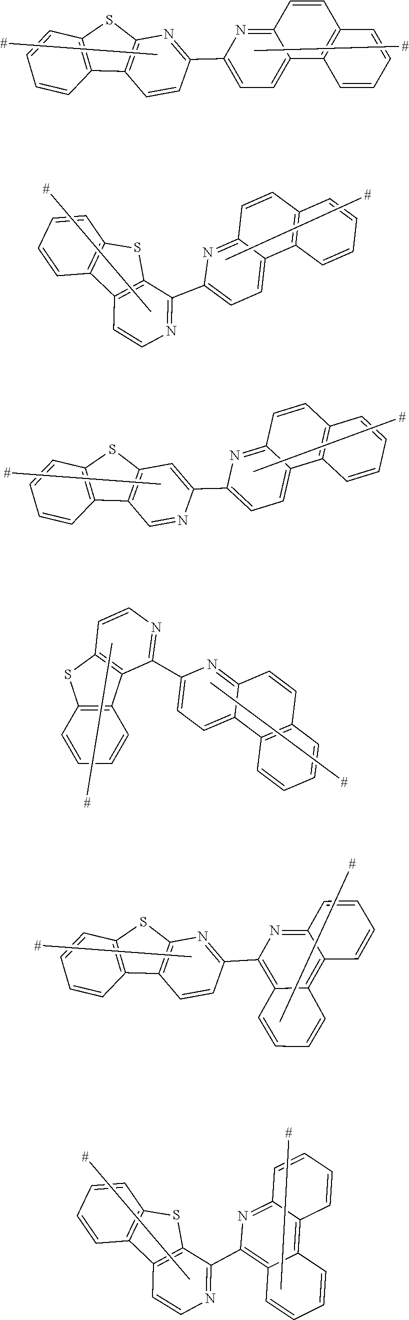

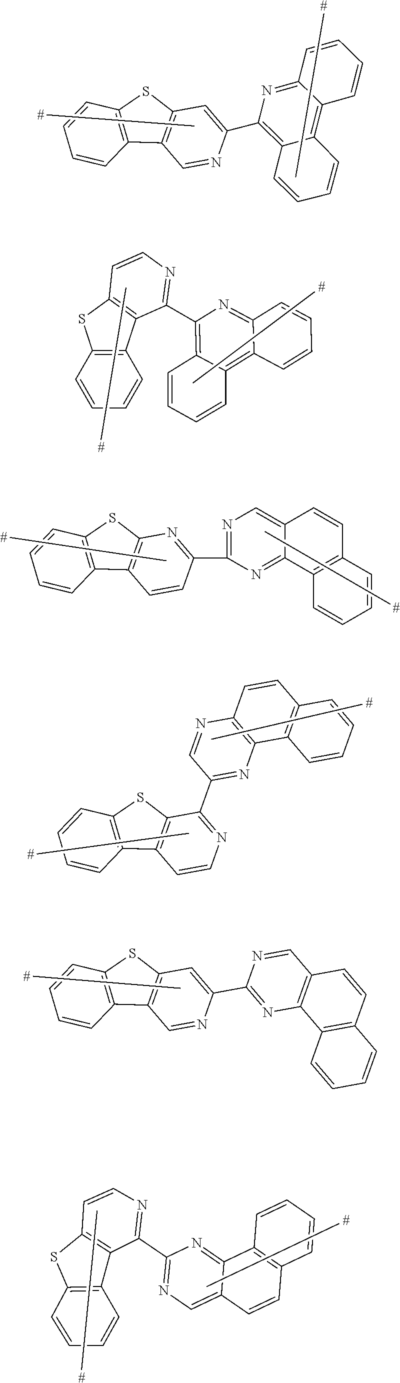

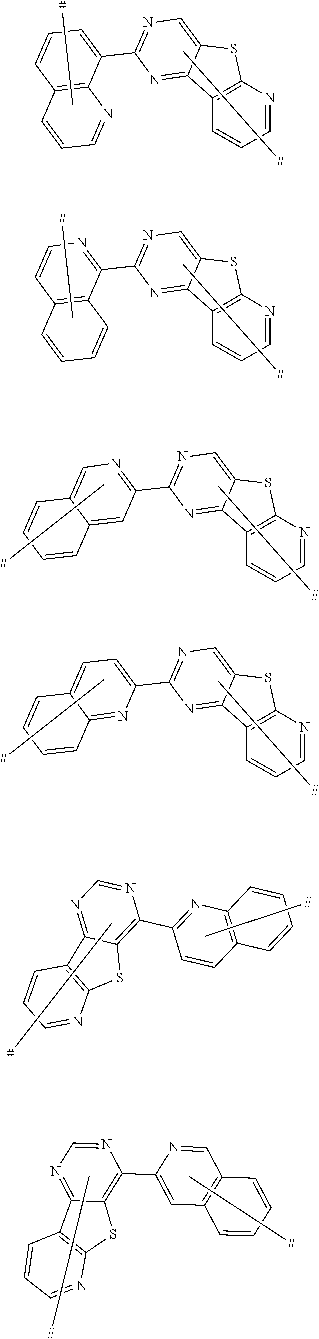

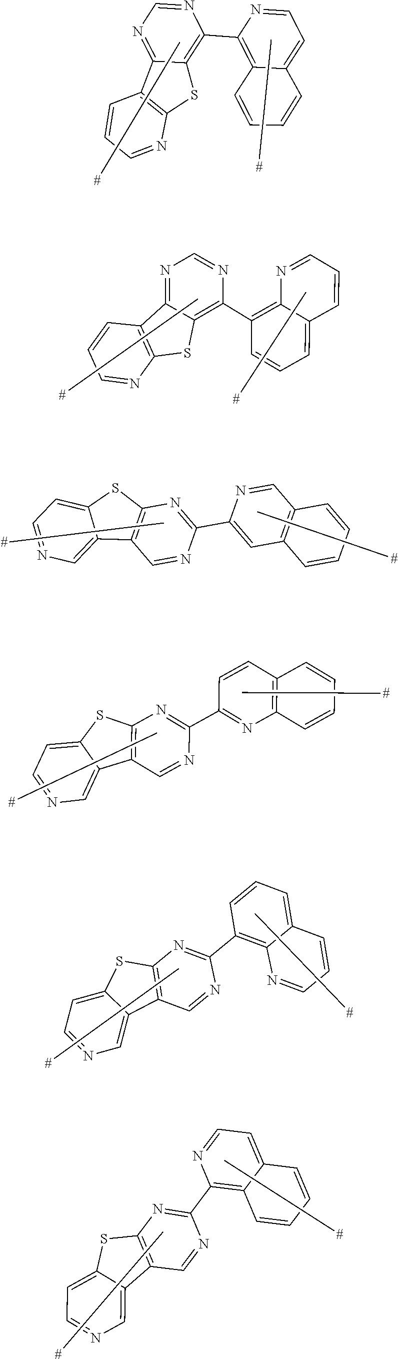

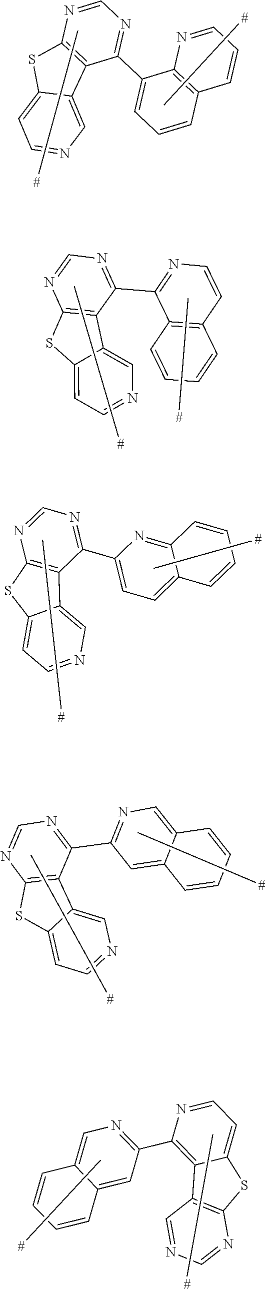

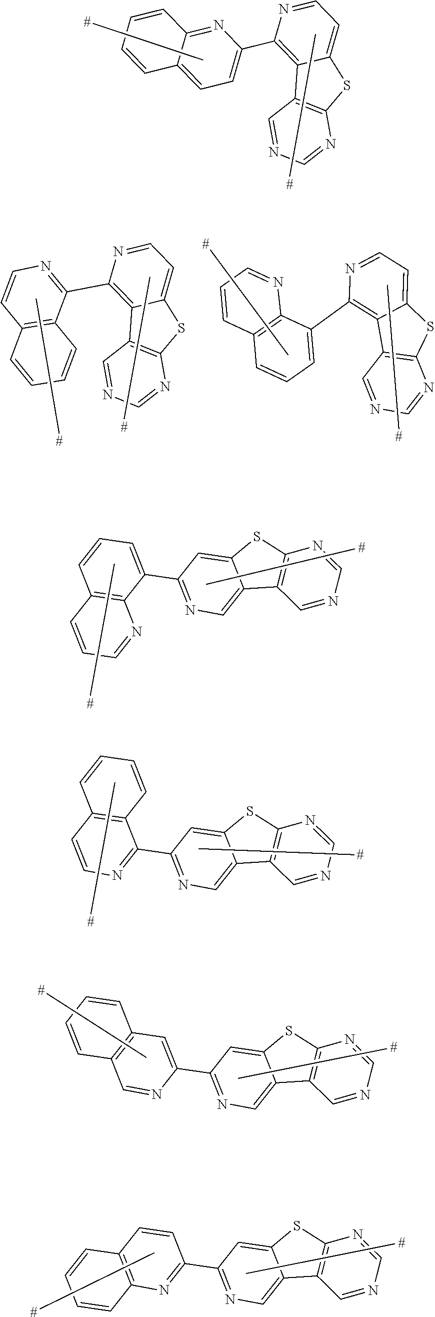

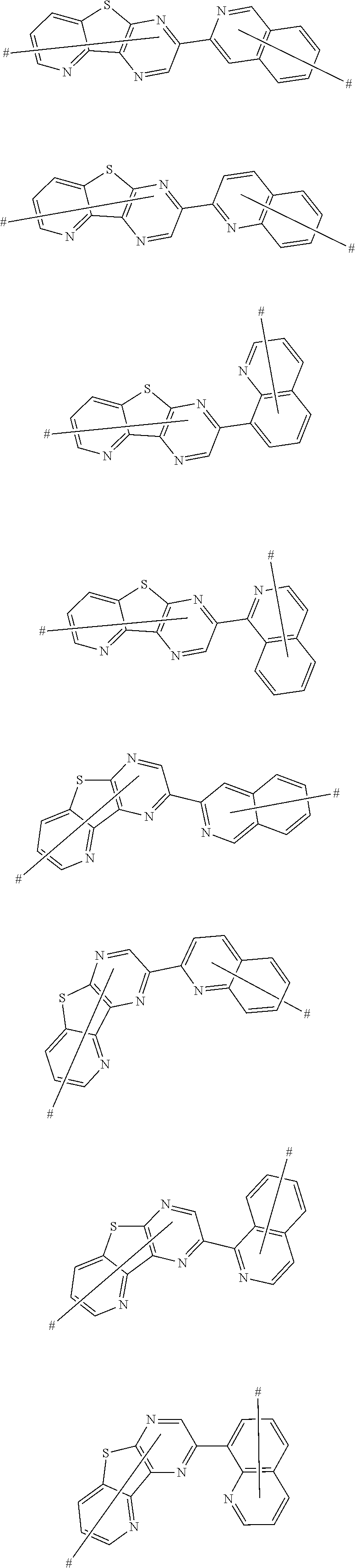

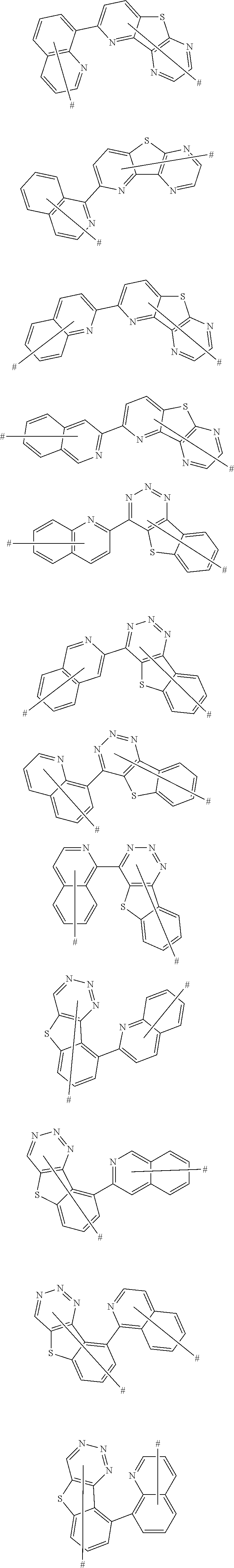

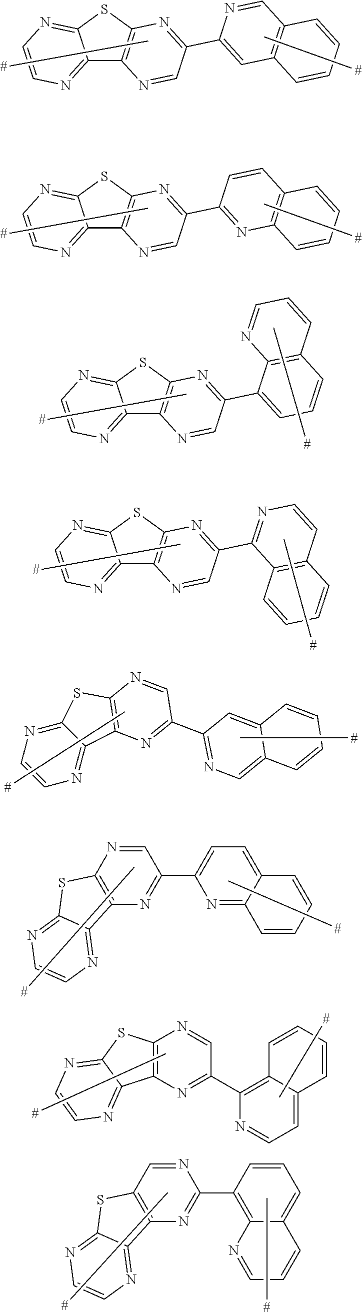

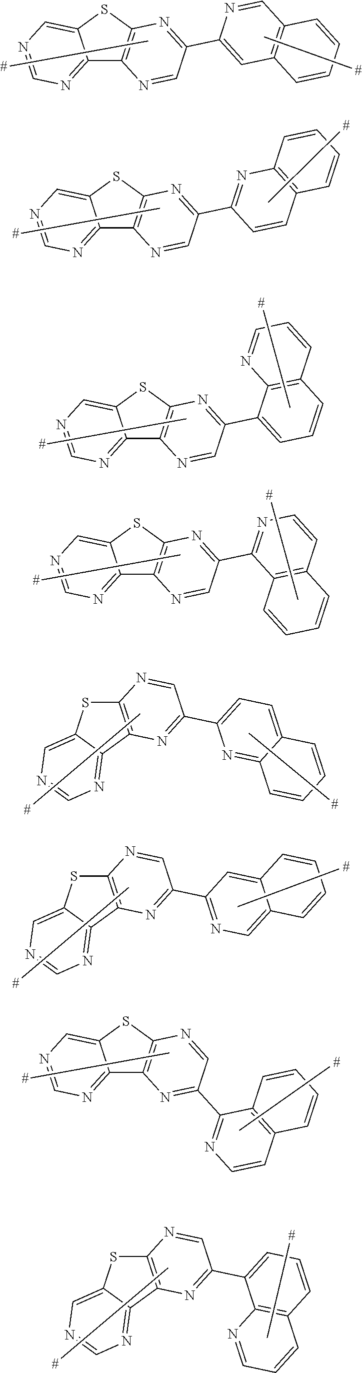

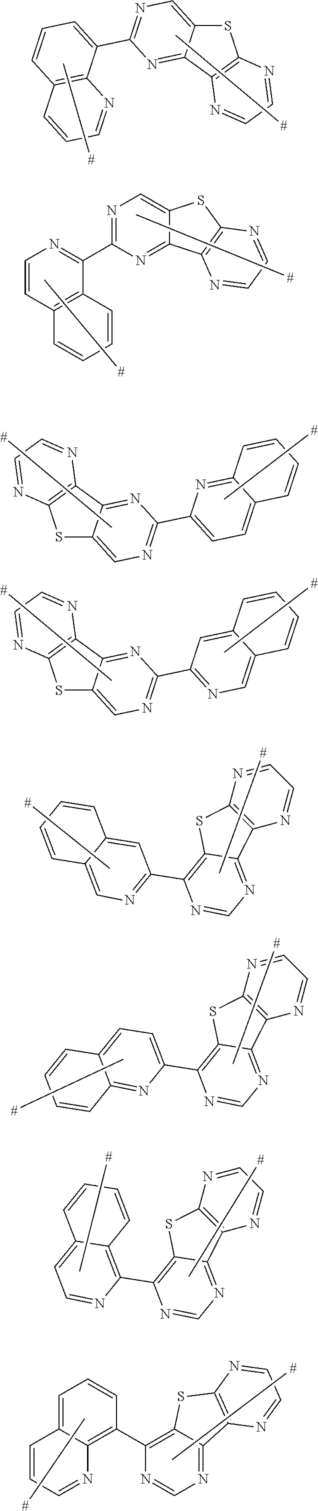

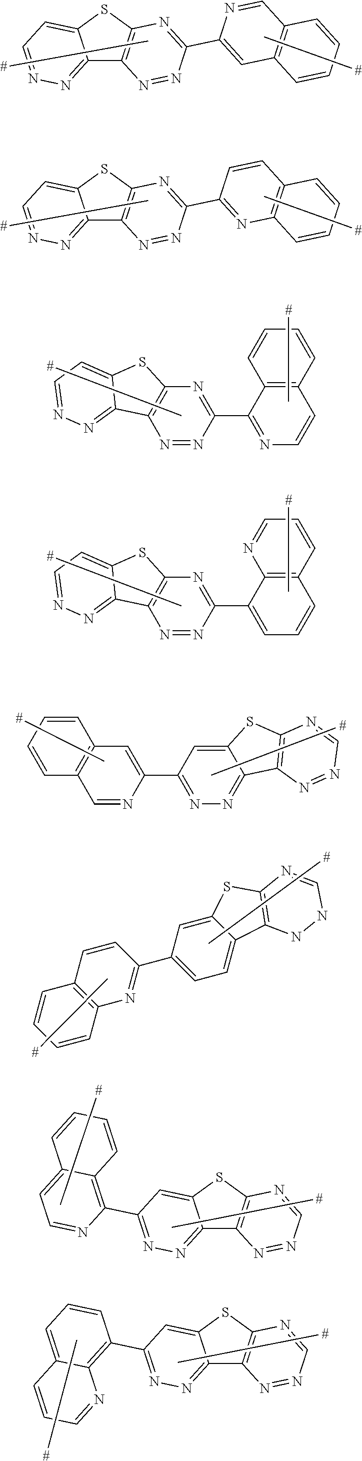

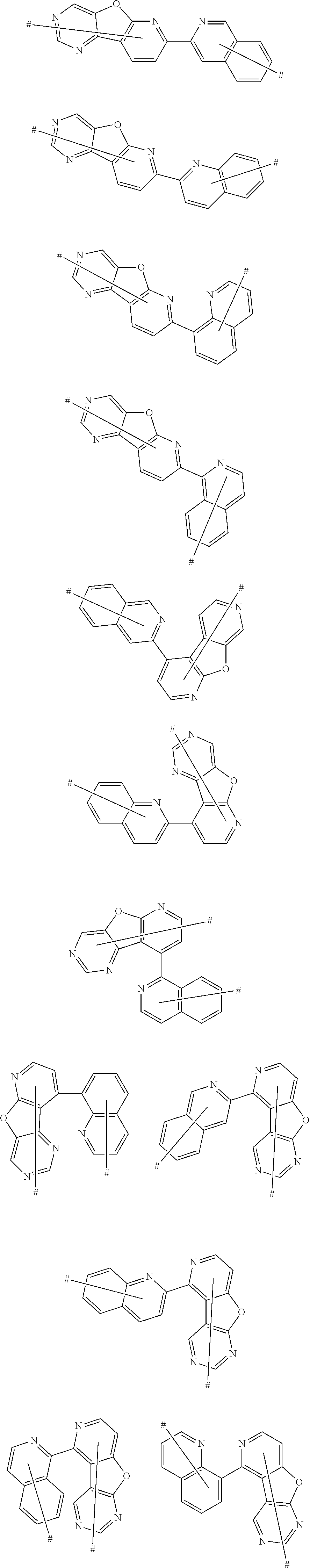

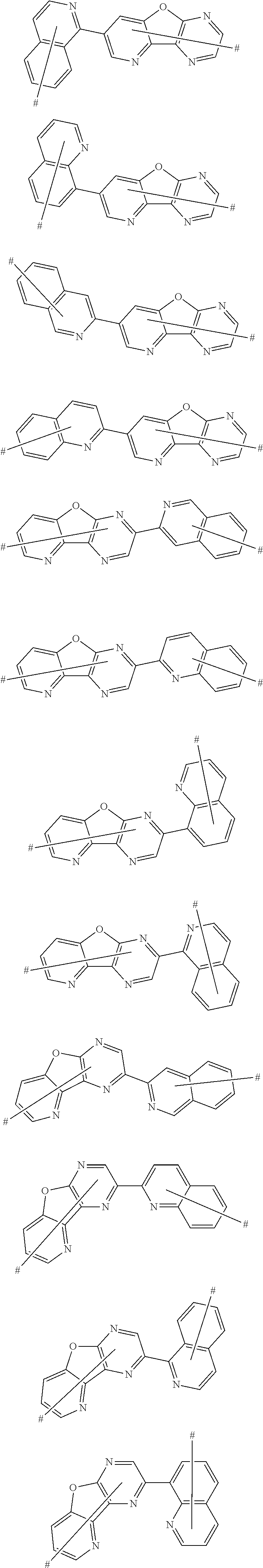

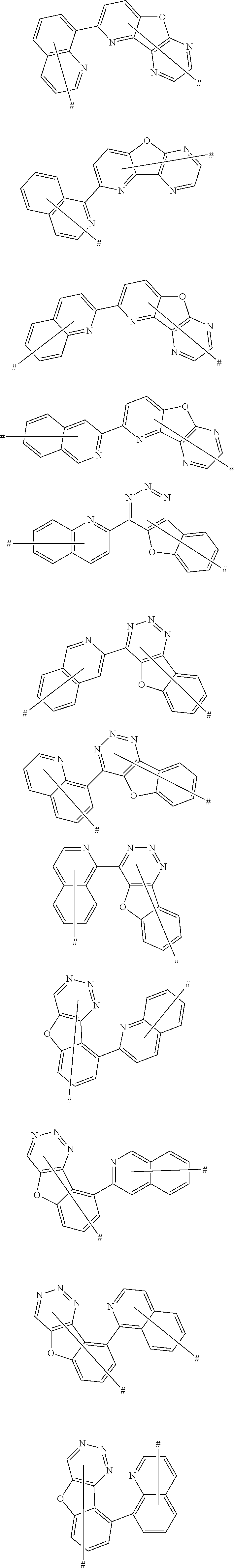

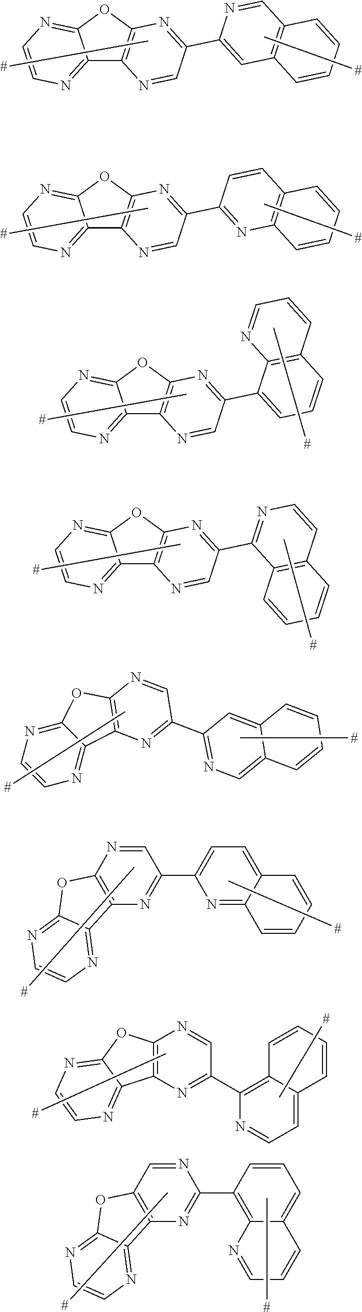

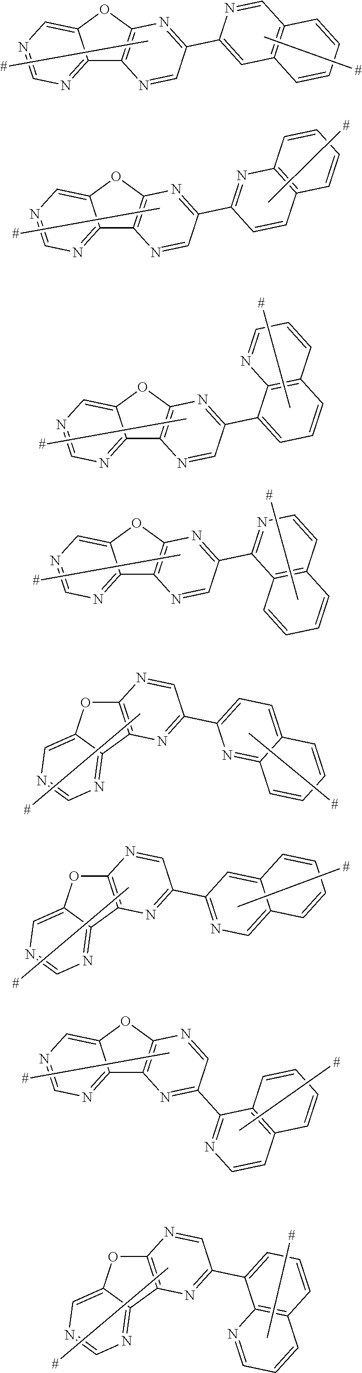

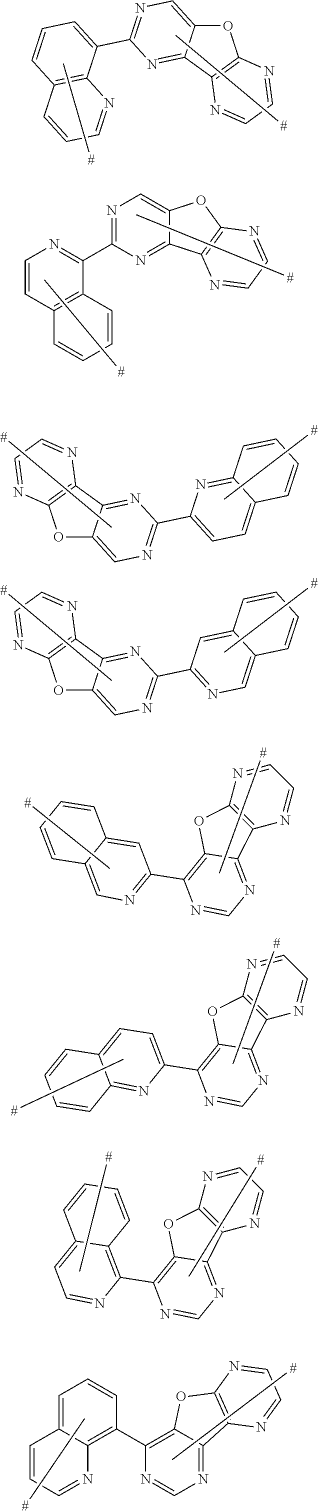

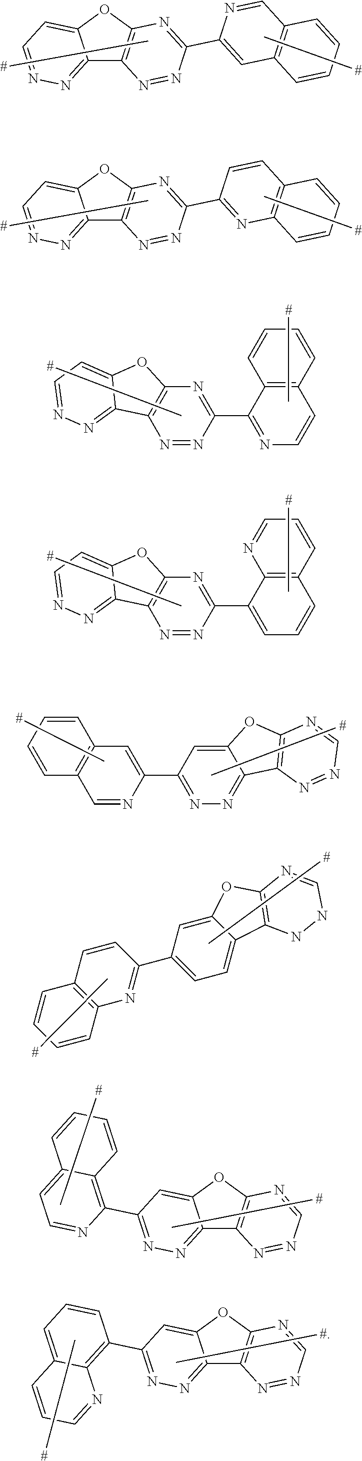

The two #in the above structural formula represent that they can be alternatively connected with L.

In some embodiments, the above heterocyclic compound has any one of the following structures:

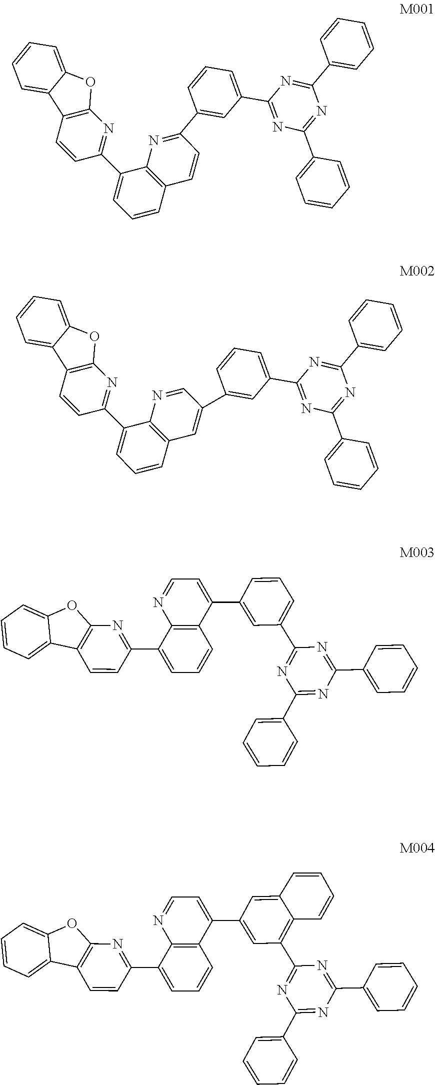 [SMILES: c1ccc(-c2nc(-c3ccccc3)nc(-c3cccc(-c4ccc5cccc(-c6ccc7c(n6)oc6ccccc67)c5n4)c3)n2)cc1 ; c1ccc(-c2nc(-c3ccccc3)nc(-c3cccc(-c4cnc5c(-c6ccc7c(n6)oc6ccccc67)cccc5c4)c3)n2)cc1 ; c1ccc(-c2nc(-c3ccccc3)nc(-c3cccc(-c4ccnc5c(-c6ccc7c(n6)oc6ccccc67)cccc45)c3)n2)cc1 ; c1ccc(-c2nc(-c3ccccc3)nc(-c3cc(-c4ccnc5c(-c6ccc7c(n6)oc6ccccc67)cccc45)cc4ccccc34)n2)cc1]

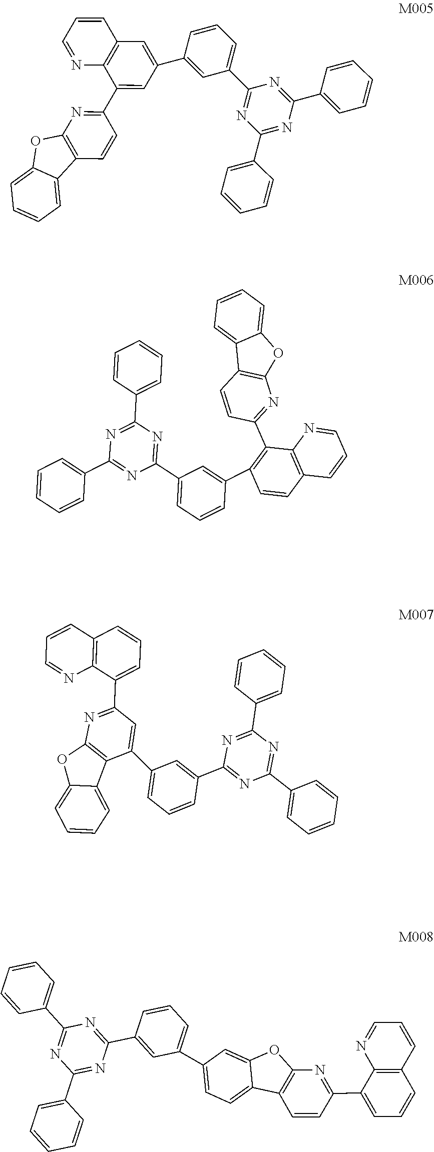 [SMILES: c1ccc(-c2nc(-c3ccccc3)nc(-c3cccc(-c4cc(-c5ccc6c(n5)oc5ccccc56)c5ncccc5c4)c3)n2)cc1 ; c1ccc(-c2nc(-c3ccccc3)nc(-c3cccc(-c4ccc5cccnc5c4-c4ccc5c(n4)oc4ccccc45)c3)n2)cc1 ; c1ccc(-c2nc(-c3ccccc3)nc(-c3cccc(-c4cc(-c5cccc6cccnc56)nc5oc6ccccc6c45)c3)n2)cc1 ; c1ccc(-c2nc(-c3ccccc3)nc(-c3cccc(-c4ccc5c(c4)oc4nc(-c6cccc7cccnc67)ccc45)c3)n2)cc1]

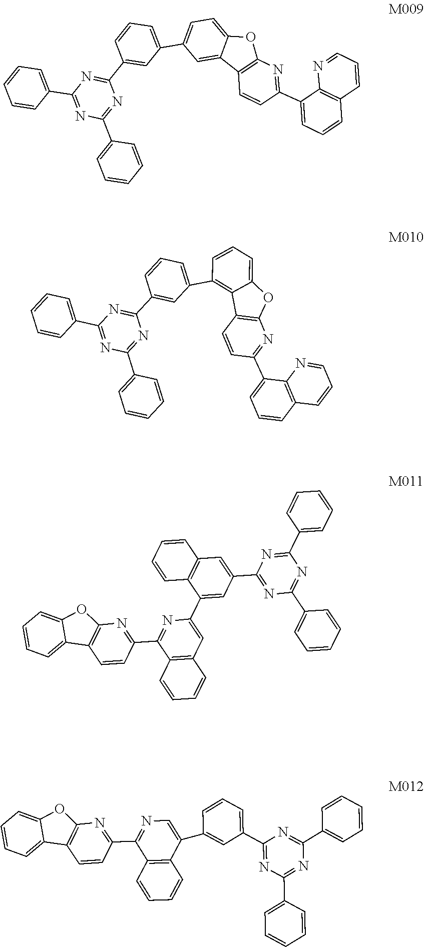 [SMILES: c1ccc(-c2nc(-c3ccccc3)nc(-c3cccc(-c4ccc5oc6nc(-c7cccc8cccnc78)ccc6c5c4)c3)n2)cc1 ; c1ccc(-c2nc(-c3ccccc3)nc(-c3cccc(-c4cccc5oc6nc(-c7cccc8cccnc78)ccc6c45)c3)n2)cc1 ; c1ccc(-c2nc(-c3ccccc3)nc(-c3cc(-c4cc5ccccc5c(-c5ccc6c(n5)oc5ccccc56)n4)c4ccccc4c3)n2)cc1 ; c1ccc(-c2nc(-c3ccccc3)nc(-c3cccc(-c4cnc(-c5ccc6c(n5)oc5ccccc56)c5ccccc45)c3)n2)cc1]

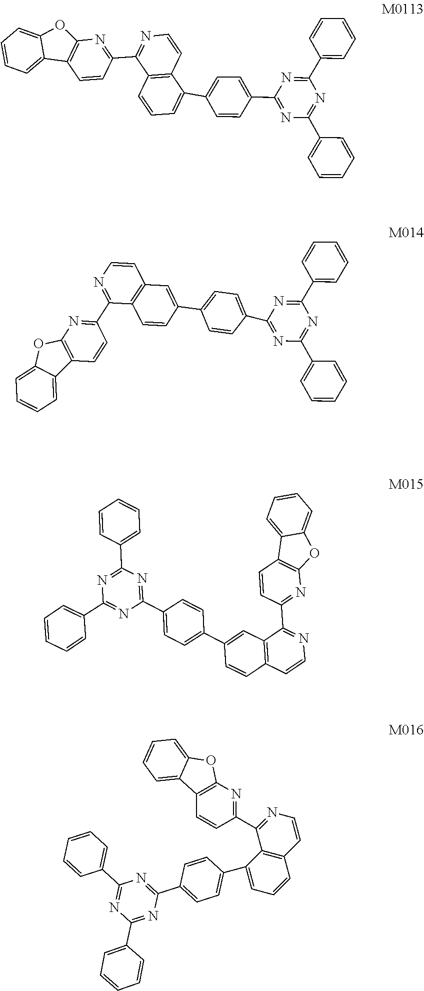 [SMILES: c1ccc(-c2nc(-c3ccccc3)nc(-c3ccc(-c4cccc5c(-c6ccc7c(n6)oc6ccccc67)nccc45)cc3)n2)cc1 ; c1ccc(-c2nc(-c3ccccc3)nc(-c3ccc(-c4ccc5c(-c6ccc7c(n6)oc6ccccc67)nccc5c4)cc3)n2)cc1 ; c1ccc(-c2nc(-c3ccccc3)nc(-c3ccc(-c4ccc5ccnc(-c6ccc7c(n6)oc6ccccc67)c5c4)cc3)n2)cc1 ; c1ccc(-c2nc(-c3ccccc3)nc(-c3ccc(-c4cccc5ccnc(-c6ccc7c(n6)oc6ccccc67)c45)cc3)n2)cc1]

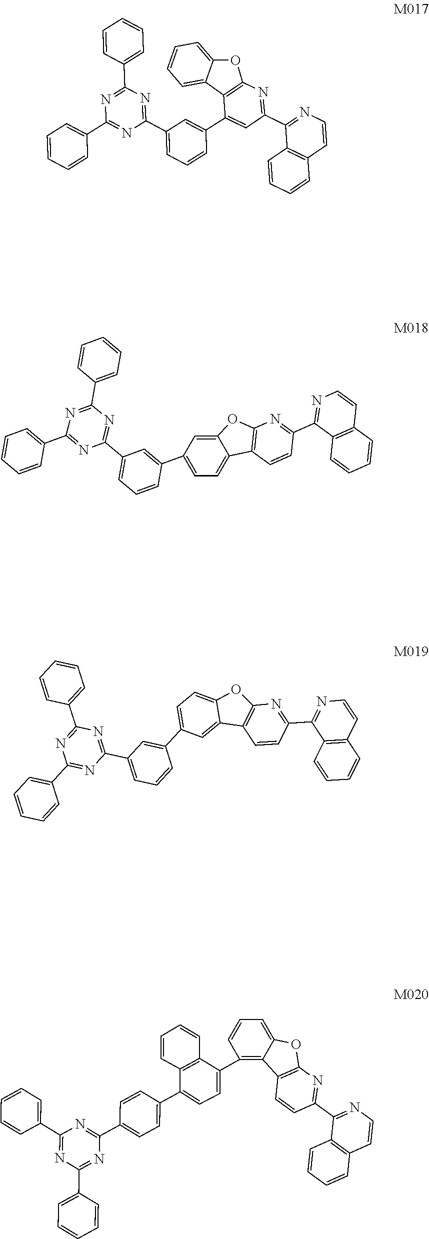 [SMILES: c1ccc(-c2nc(-c3ccccc3)nc(-c3cccc(-c4cc(-c5nccc6ccccc56)nc5oc6ccccc6c45)c3)n2)cc1 ; c1ccc(-c2nc(-c3ccccc3)nc(-c3cccc(-c4ccc5c(c4)oc4nc(-c6nccc7ccccc67)ccc45)c3)n2)cc1 ; c1ccc(-c2nc(-c3ccccc3)nc(-c3cccc(-c4ccc5oc6nc(-c7nccc8ccccc78)ccc6c5c4)c3)n2)cc1 ; c1ccc(-c2nc(-c3ccccc3)nc(-c3ccc(-c4ccc(-c5cccc6oc7nc(-c8nccc9ccccc89)ccc7c56)c5ccccc45)cc3)n2)cc1]

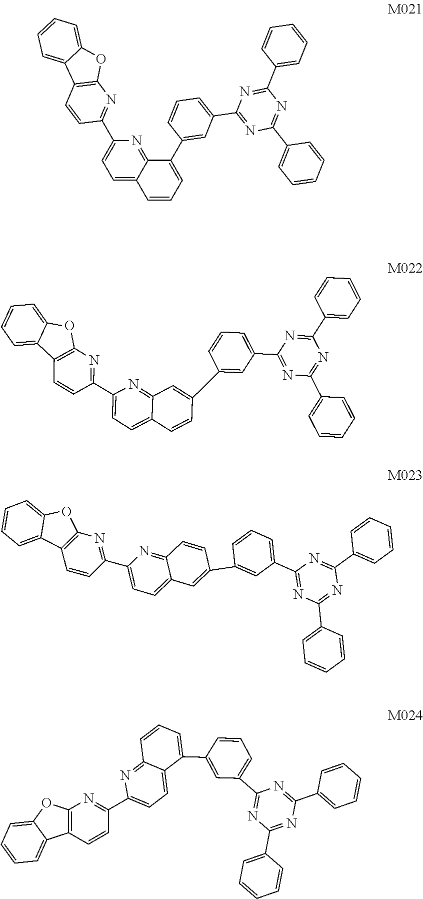 [SMILES: c1ccc(-c2nc(-c3ccccc3)nc(-c3cccc(-c4cccc5ccc(-c6ccc7c(n6)oc6ccccc67)nc45)c3)n2)cc1 ; c1ccc(-c2nc(-c3ccccc3)nc(-c3cccc(-c4ccc5ccc(-c6ccc7c(n6)oc6ccccc67)nc5c4)c3)n2)cc1 ; c1ccc(-c2nc(-c3ccccc3)nc(-c3cccc(-c4ccc5nc(-c6ccc7c(n6)oc6ccccc67)ccc5c4)c3)n2)cc1 ; c1ccc(-c2nc(-c3ccccc3)nc(-c3cccc(-c4cccc5nc(-c6ccc7c(n6)oc6ccccc67)ccc45)c3)n2)cc1]

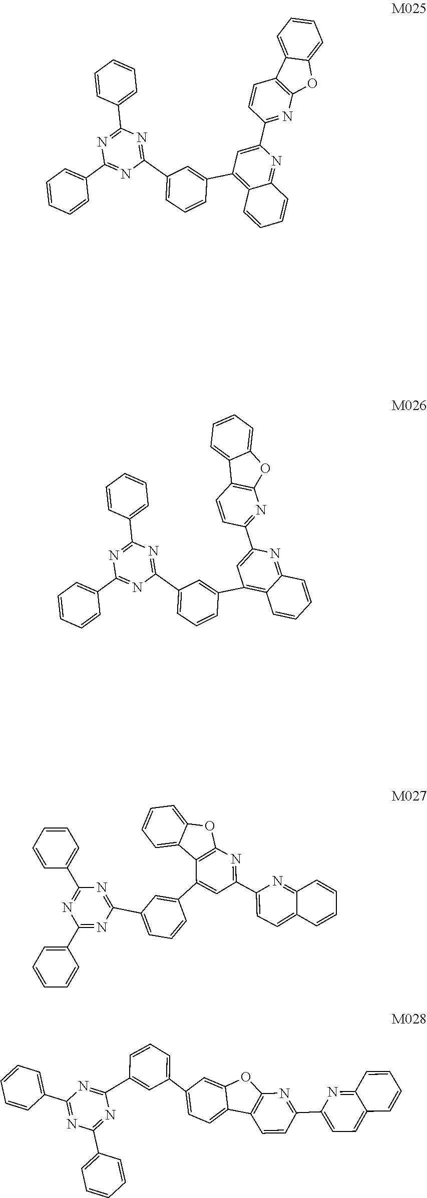 [SMILES: c1ccc(-c2nc(-c3ccccc3)nc(-c3cccc(-c4cc(-c5ccc6c(n5)oc5ccccc56)nc5ccccc45)c3)n2)cc1 ; c1ccc(-c2nc(-c3ccccc3)nc(-c3cccc(-c4cc(-c5ccc6c(n5)oc5ccccc56)nc5ccccc45)c3)n2)cc1 ; c1ccc(-c2nc(-c3ccccc3)nc(-c3cccc(-c4cc(-c5ccc6ccccc6n5)nc5oc6ccccc6c45)c3)n2)cc1 ; c1ccc(-c2nc(-c3ccccc3)nc(-c3cccc(-c4ccc5c(c4)oc4nc(-c6ccc7ccccc7n6)ccc45)c3)n2)cc1]

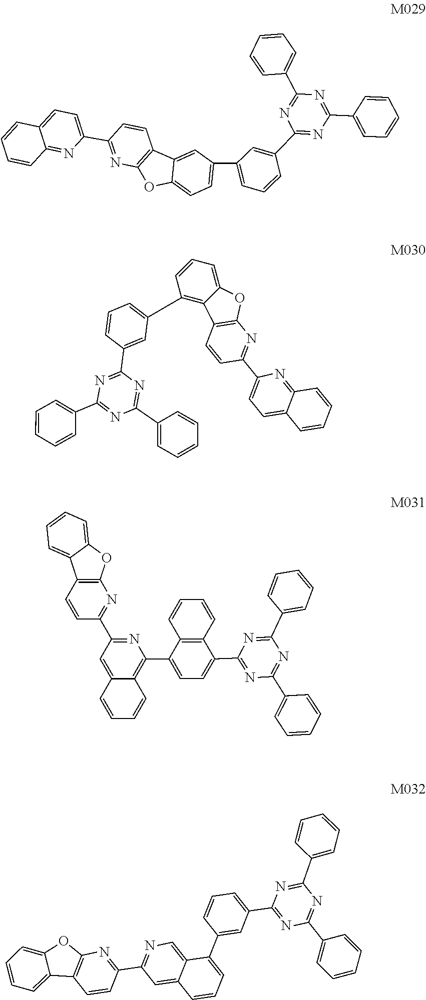

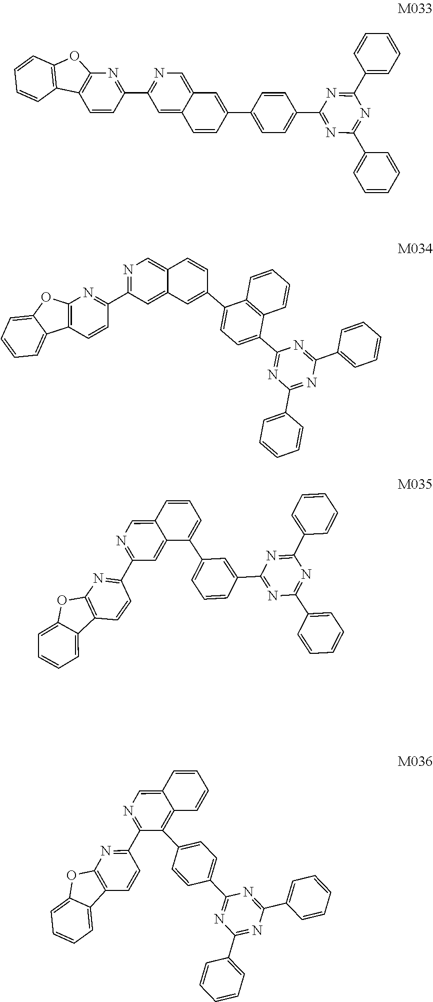 [SMILES: c1ccc(-c2nc(-c3ccccc3)nc(-c3ccc(-c4ccc5cc(-c6ccc7c(n6)oc6ccccc67)ncc5c4)cc3)n2)cc1 ; c1ccc(-c2nc(-c3ccccc3)nc(-c3ccc(-c4ccc5cnc(-c6ccc7c(n6)oc6ccccc67)cc5c4)c4ccccc34)n2)cc1 ; c1ccc(-c2nc(-c3ccccc3)nc(-c3cccc(-c4cccc5cnc(-c6ccc7c(n6)oc6ccccc67)cc45)c3)n2)cc1 ; c1ccc(-c2nc(-c3ccccc3)nc(-c3ccc(-c4c(-c5ccc6c(n5)oc5ccccc56)ncc5ccccc45)cc3)n2)cc1]

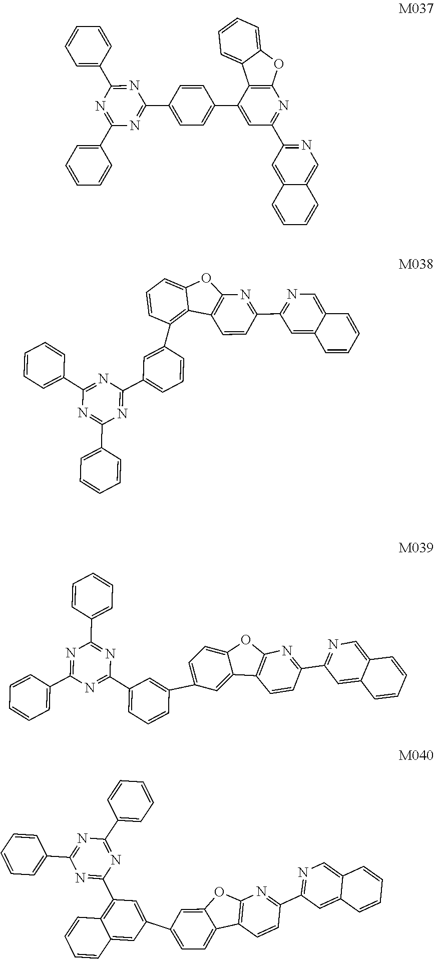 [SMILES: c1ccc(-c2nc(-c3ccccc3)nc(-c3ccc(-c4cc(-c5cc6ccccc6cn5)nc5oc6ccccc6c45)cc3)n2)cc1 ; c1ccc(-c2nc(-c3ccccc3)nc(-c3cccc(-c4cccc5oc6nc(-c7cc8ccccc8cn7)ccc6c45)c3)n2)cc1 ; c1ccc(-c2nc(-c3ccccc3)nc(-c3cccc(-c4ccc5oc6nc(-c7cc8ccccc8cn7)ccc6c5c4)c3)n2)cc1 ; c1ccc(-c2nc(-c3ccccc3)nc(-c3cc(-c4ccc5c(c4)oc4nc(-c6cc7ccccc7cn6)ccc45)cc4ccccc34)n2)cc1]

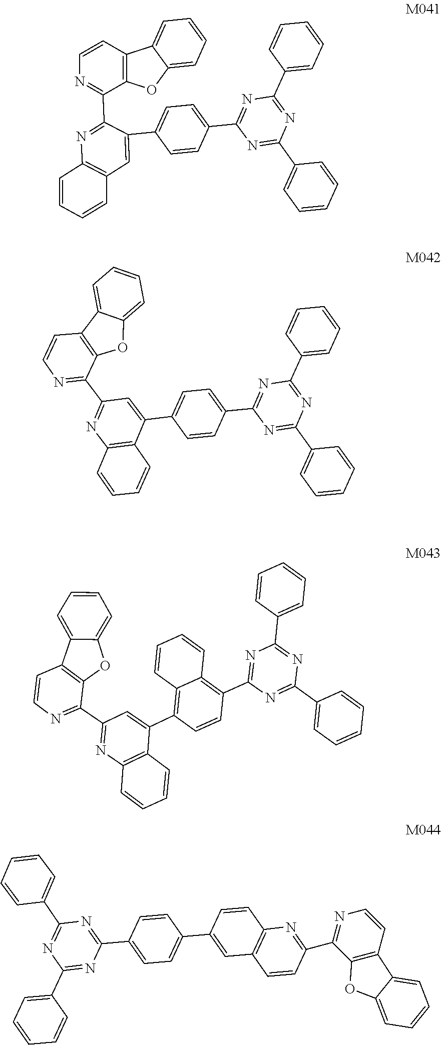 [SMILES: c1ccc(-c2nc(-c3ccccc3)nc(-c3ccc(-c4cc5ccccc5nc4-c4nccc5c4oc4ccccc45)cc3)n2)cc1 ; c1ccc(-c2nc(-c3ccccc3)nc(-c3ccc(-c4cc(-c5nccc6c5oc5ccccc56)nc5ccccc45)cc3)n2)cc1 ; c1ccc(-c2nc(-c3ccccc3)nc(-c3ccc(-c4cc(-c5nccc6c5oc5ccccc56)nc5ccccc45)c4ccccc34)n2)cc1 ; c1ccc(-c2nc(-c3ccccc3)nc(-c3ccc(-c4ccc5nc(-c6nccc7c6oc6ccccc67)ccc5c4)cc3)n2)cc1]

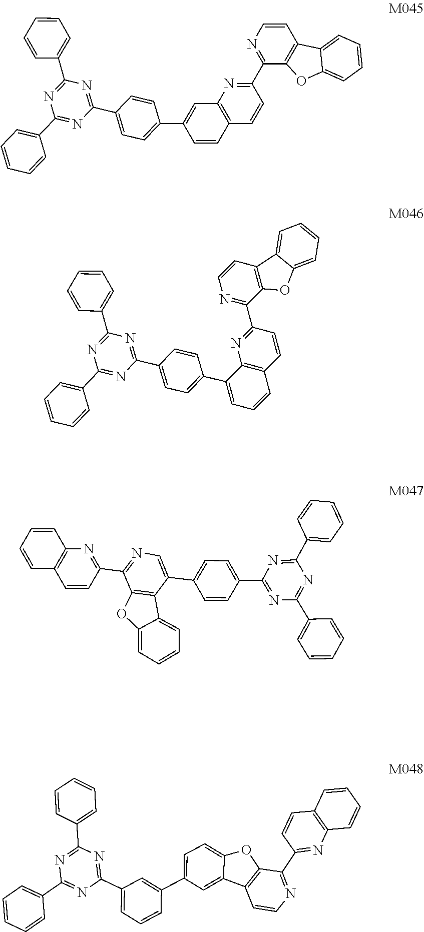 [SMILES: c1ccc(-c2nc(-c3ccccc3)nc(-c3ccc(-c4ccc5ccc(-c6nccc7c6oc6ccccc67)nc5c4)cc3)n2)cc1 ; c1ccc(-c2nc(-c3ccccc3)nc(-c3ccc(-c4cccc5ccc(-c6nccc7c6oc6ccccc67)nc45)cc3)n2)cc1 ; c1ccc(-c2nc(-c3ccccc3)nc(-c3ccc(-c4cnc(-c5ccc6ccccc6n5)c5oc6ccccc6c45)cc3)n2)cc1 ; c1ccc(-c2nc(-c3ccccc3)nc(-c3cccc(-c4ccc5oc6c(-c7ccc8ccccc8n7)nccc6c5c4)c3)n2)cc1]

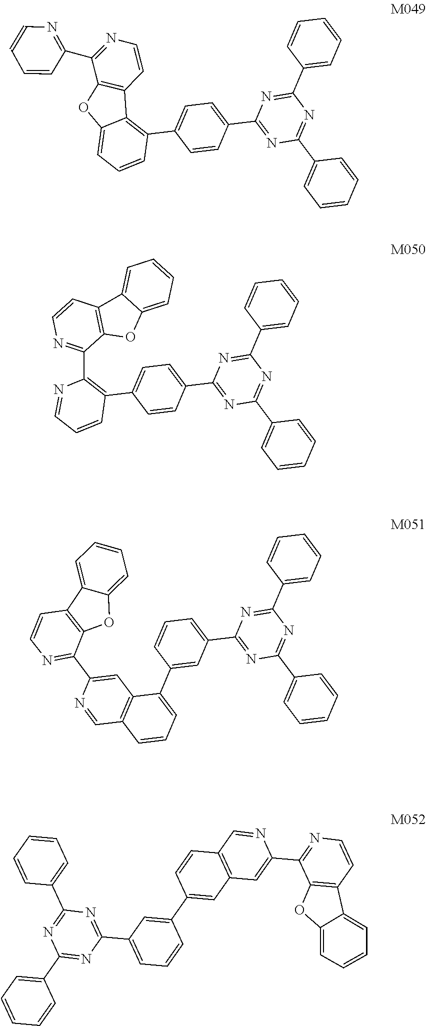 [SMILES: c1ccc(-c2nc(-c3ccccc3)nc(-c3ccc(-c4cccc5oc6c(-c7ccccn7)nccc6c45)cc3)n2)cc1 ; c1ccc(-c2nc(-c3ccccc3)nc(-c3ccc(-c4cccnc4-c4nccc5c4oc4ccccc45)cc3)n2)cc1 ; c1ccc(-c2nc(-c3ccccc3)nc(-c3cccc(-c4cccc5cnc(-c6nccc7c6oc6ccccc67)cc45)c3)n2)cc1 ; c1ccc(-c2nc(-c3ccccc3)nc(-c3cccc(-c4ccc5cnc(-c6nccc7c6oc6ccccc67)cc5c4)c3)n2)cc1]

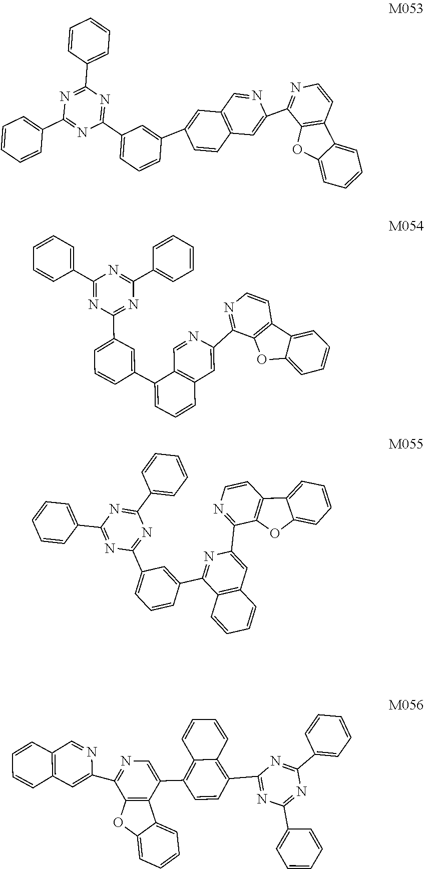 [SMILES: c1ccc(-c2nc(-c3ccccc3)nc(-c3cccc(-c4ccc5cc(-c6nccc7c6oc6ccccc67)ncc5c4)c3)n2)cc1 ; c1ccc(-c2nc(-c3ccccc3)nc(-c3cccc(-c4cccc5cc(-c6nccc7c6oc6ccccc67)ncc45)c3)n2)cc1 ; c1ccc(-c2nc(-c3ccccc3)nc(-c3cccc(-c4nc(-c5nccc6c5oc5ccccc56)cc5ccccc45)c3)n2)cc1 ; c1ccc(-c2nc(-c3ccccc3)nc(-c3ccc(-c4cnc(-c5cc6ccccc6cn5)c5oc6ccccc6c45)c4ccccc34)n2)cc1]

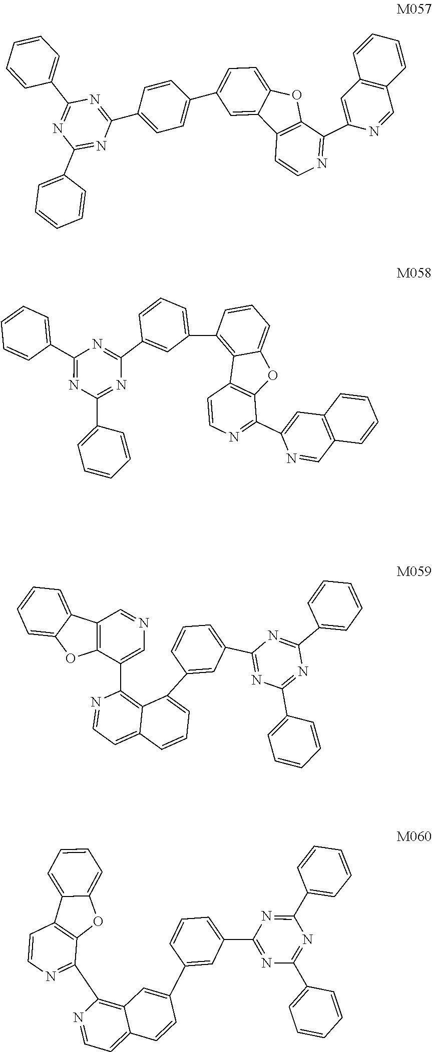 [SMILES: c1ccc(-c2nc(-c3ccccc3)nc(-c3ccc(-c4ccc5oc6c(-c7cc8ccccc8cn7)nccc6c5c4)cc3)n2)cc1 ; c1ccc(-c2nc(-c3ccccc3)nc(-c3cccc(-c4cccc5oc6c(-c7cc8ccccc8cn7)nccc6c45)c3)n2)cc1 ; c1ccc(-c2nc(-c3ccccc3)nc(-c3cccc(-c4cccc5ccnc(-c6cncc7c6oc6ccccc67)c45)c3)n2)cc1 ; c1ccc(-c2nc(-c3ccccc3)nc(-c3cccc(-c4ccc5ccnc(-c6nccc7c6oc6ccccc67)c5c4)c3)n2)cc1]

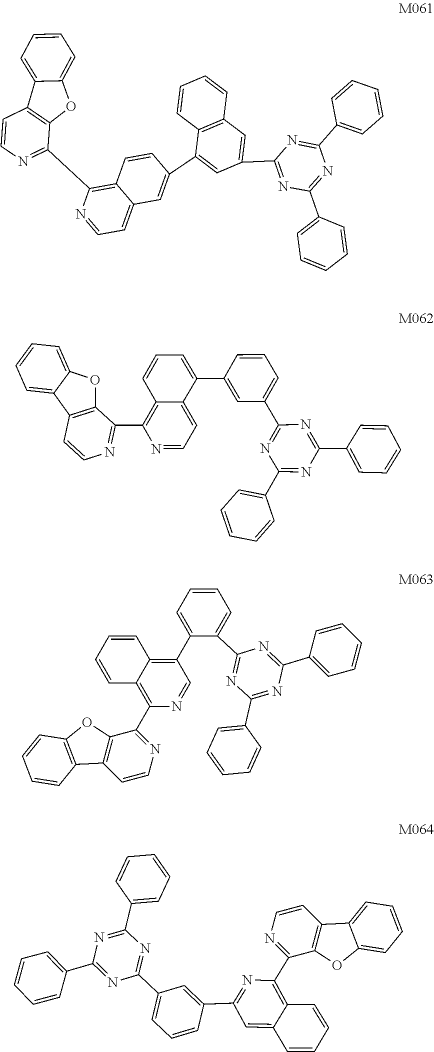 [SMILES: c1ccc(-c2nc(-c3ccccc3)nc(-c3cc(-c4ccc5c(-c6nccc7c6oc6ccccc67)nccc5c4)c4ccccc4c3)n2)cc1 ; c1ccc(-c2nc(-c3ccccc3)nc(-c3cccc(-c4cccc5c(-c6nccc7c6oc6ccccc67)nccc45)c3)n2)cc1 ; c1ccc(-c2nc(-c3ccccc3)nc(-c3ccccc3-c3cnc(-c4nccc5c4oc4ccccc45)c4ccccc34)n2)cc1 ; c1ccc(-c2nc(-c3ccccc3)nc(-c3cccc(-c4cc5ccccc5c(-c5nccc6c5oc5ccccc56)n4)c3)n2)cc1]

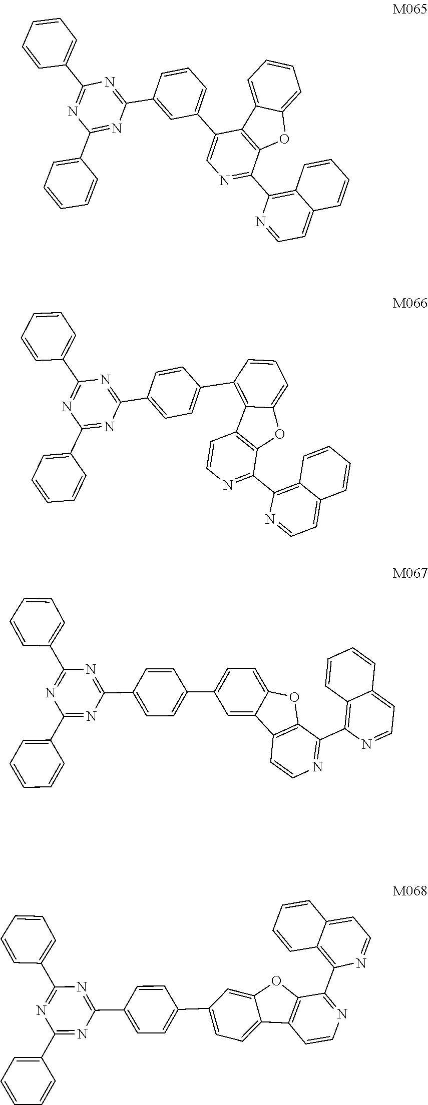 [SMILES: c1ccc(-c2nc(-c3ccccc3)nc(-c3cccc(-c4cnc(-c5nccc6ccccc56)c5oc6ccccc6c45)c3)n2)cc1 ; c1ccc(-c2nc(-c3ccccc3)nc(-c3ccc(-c4cccc5oc6c(-c7nccc8ccccc78)nccc6c45)cc3)n2)cc1 ; c1ccc(-c2nc(-c3ccccc3)nc(-c3ccc(-c4ccc5oc6c(-c7nccc8ccccc78)nccc6c5c4)cc3)n2)cc1 ; c1ccc(-c2nc(-c3ccccc3)nc(-c3ccc(-c4ccc5c(c4)oc4c(-c6nccc7ccccc67)nccc45)cc3)n2)cc1]

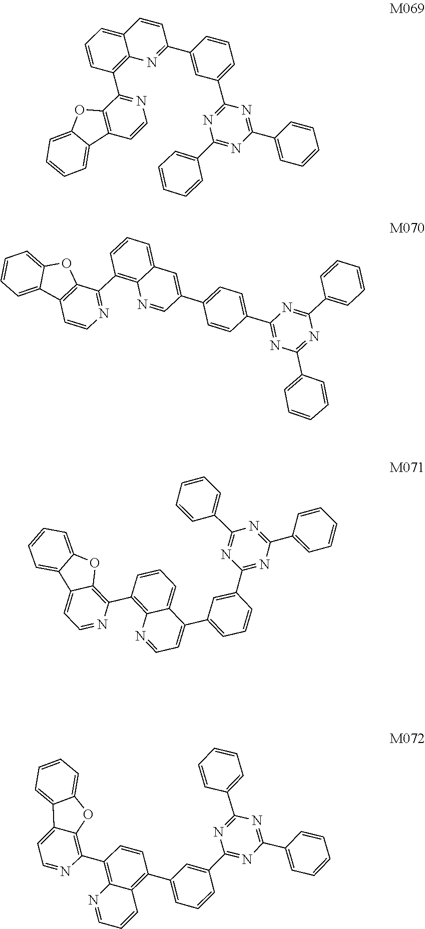 [SMILES: c1ccc(-c2nc(-c3ccccc3)nc(-c3cccc(-c4ccc5cccc(-c6nccc7c6oc6ccccc67)c5n4)c3)n2)cc1 ; c1ccc(-c2nc(-c3ccccc3)nc(-c3ccc(-c4cnc5c(-c6nccc7c6oc6ccccc67)cccc5c4)cc3)n2)cc1 ; c1ccc(-c2nc(-c3ccccc3)nc(-c3cccc(-c4ccnc5c(-c6nccc7c6oc6ccccc67)cccc45)c3)n2)cc1 ; c1ccc(-c2nc(-c3ccccc3)nc(-c3cccc(-c4ccc(-c5nccc6c5oc5ccccc56)c5ncccc45)c3)n2)cc1]

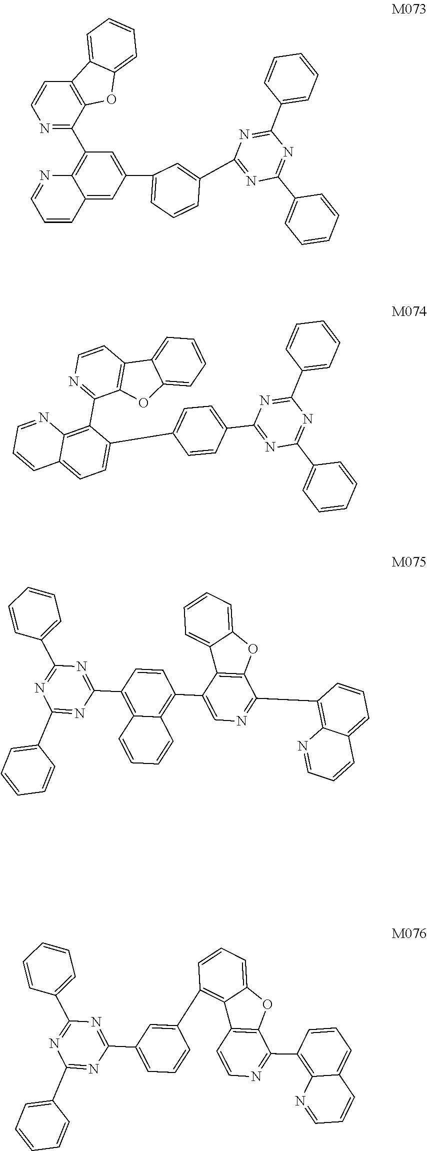 [SMILES: c1ccc(-c2nc(-c3ccccc3)nc(-c3cccc(-c4cc(-c5nccc6c5oc5ccccc56)c5ncccc5c4)c3)n2)cc1 ; c1ccc(-c2nc(-c3ccccc3)nc(-c3ccc(-c4ccc5cccnc5c4-c4nccc5c4oc4ccccc45)cc3)n2)cc1 ; c1ccc(-c2nc(-c3ccccc3)nc(-c3ccc(-c4cnc(-c5cccc6cccnc56)c5oc6ccccc6c45)c4ccccc34)n2)cc1 ; c1ccc(-c2nc(-c3ccccc3)nc(-c3cccc(-c4cccc5oc6c(-c7cccc8cccnc78)nccc6c45)c3)n2)cc1]

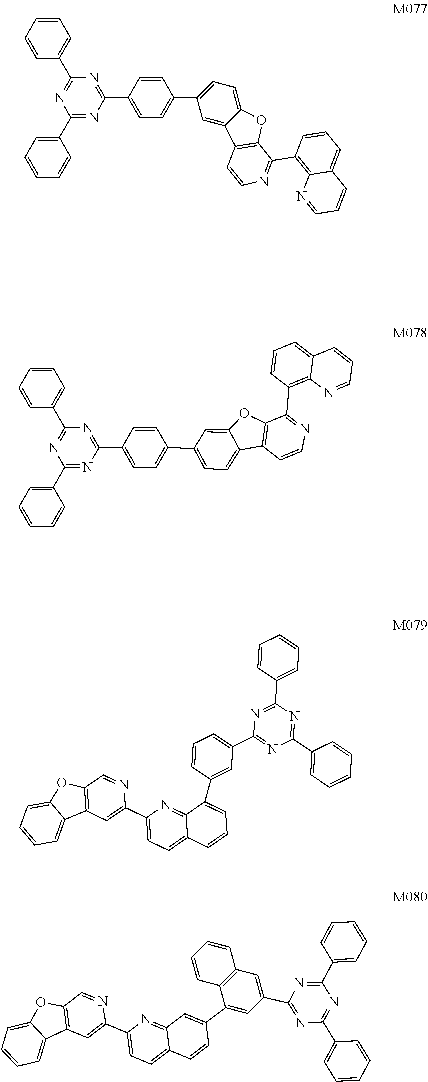 [SMILES: c1ccc(-c2nc(-c3ccccc3)nc(-c3ccc(-c4ccc5oc6c(-c7cccc8cccnc78)nccc6c5c4)cc3)n2)cc1 ; c1ccc(-c2nc(-c3ccccc3)nc(-c3ccc(-c4ccc5c(c4)oc4c(-c6cccc7cccnc67)nccc45)cc3)n2)cc1 ; c1ccc(-c2nc(-c3ccccc3)nc(-c3cccc(-c4cccc5ccc(-c6cc7c(cn6)oc6ccccc67)nc45)c3)n2)cc1 ; c1ccc(-c2nc(-c3ccccc3)nc(-c3cc(-c4ccc5ccc(-c6cc7c(cn6)oc6ccccc67)nc5c4)c4ccccc4c3)n2)cc1]

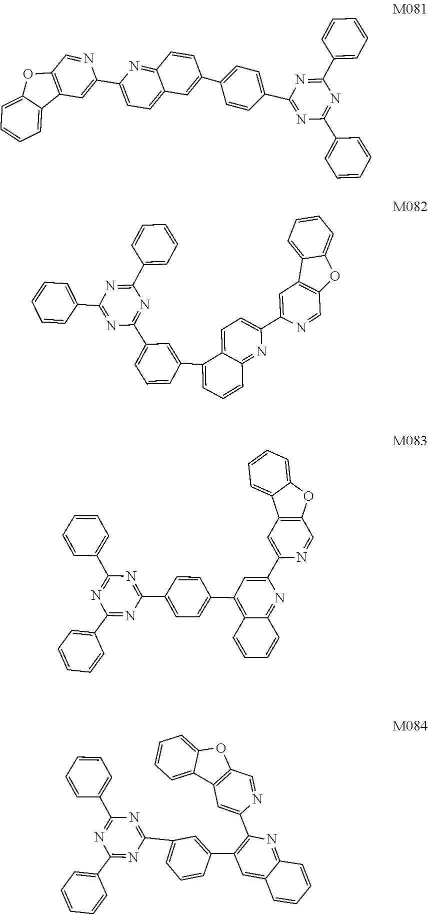 [SMILES: c1ccc(-c2nc(-c3ccccc3)nc(-c3ccc(-c4ccc5nc(-c6cc7c(cn6)oc6ccccc67)ccc5c4)cc3)n2)cc1 ; c1ccc(-c2nc(-c3ccccc3)nc(-c3cccc(-c4cccc5nc(-c6cc7c(cn6)oc6ccccc67)ccc45)c3)n2)cc1 ; c1ccc(-c2nc(-c3ccccc3)nc(-c3ccc(-c4cc(-c5cc6c(cn5)oc5ccccc56)nc5ccccc45)cc3)n2)cc1 ; c1ccc(-c2nc(-c3ccccc3)nc(-c3cccc(-c4cc5ccccc5nc4-c4cc5c(cn4)oc4ccccc45)c3)n2)cc1]

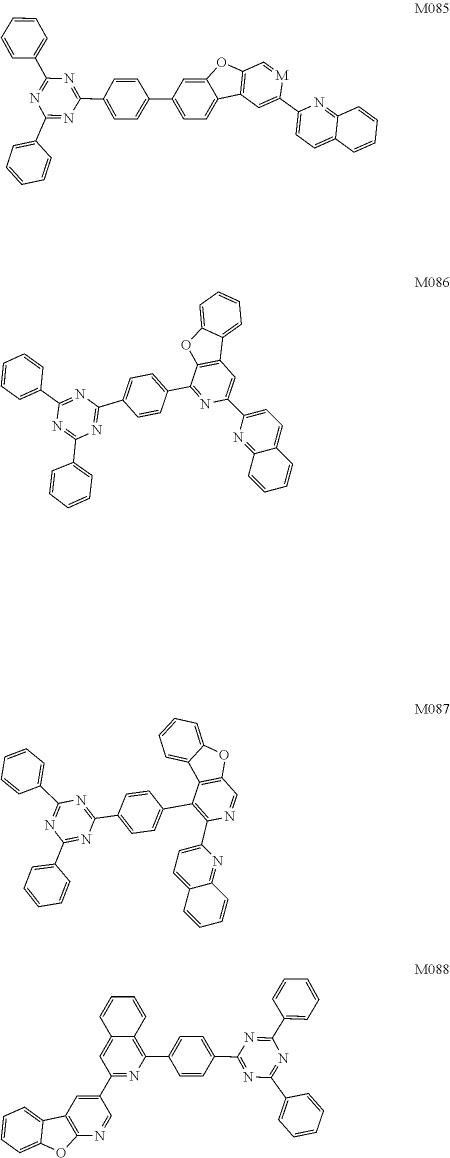

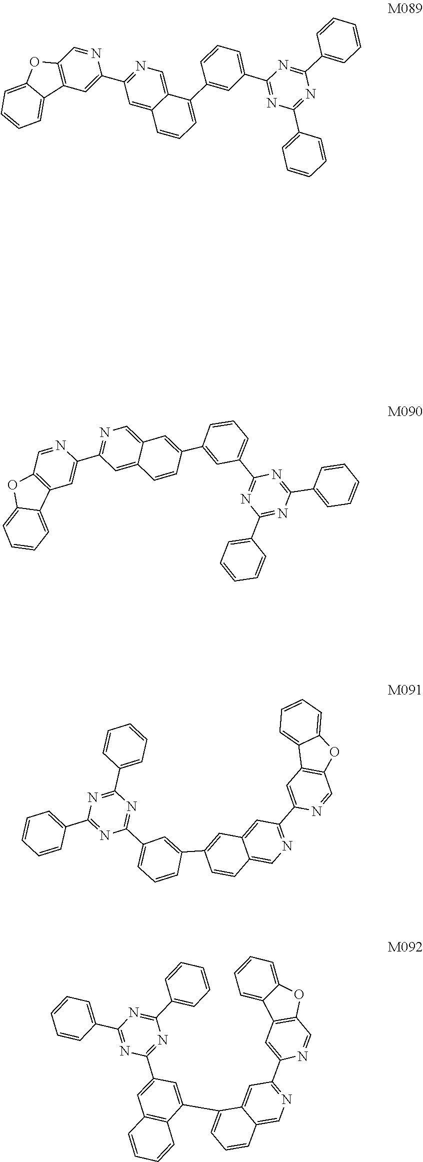 [SMILES: c1ccc(-c2nc(-c3ccccc3)nc(-c3cccc(-c4cccc5cc(-c6cc7c(cn6)oc6ccccc67)ncc45)c3)n2)cc1 ; c1ccc(-c2nc(-c3ccccc3)nc(-c3cccc(-c4ccc5cc(-c6cc7c(cn6)oc6ccccc67)ncc5c4)c3)n2)cc1 ; c1ccc(-c2nc(-c3ccccc3)nc(-c3cccc(-c4ccc5cnc(-c6cc7c(cn6)oc6ccccc67)cc5c4)c3)n2)cc1 ; c1ccc(-c2nc(-c3ccccc3)nc(-c3cc(-c4cccc5cnc(-c6cc7c(cn6)oc6ccccc67)cc45)c4ccccc4c3)n2)cc1]

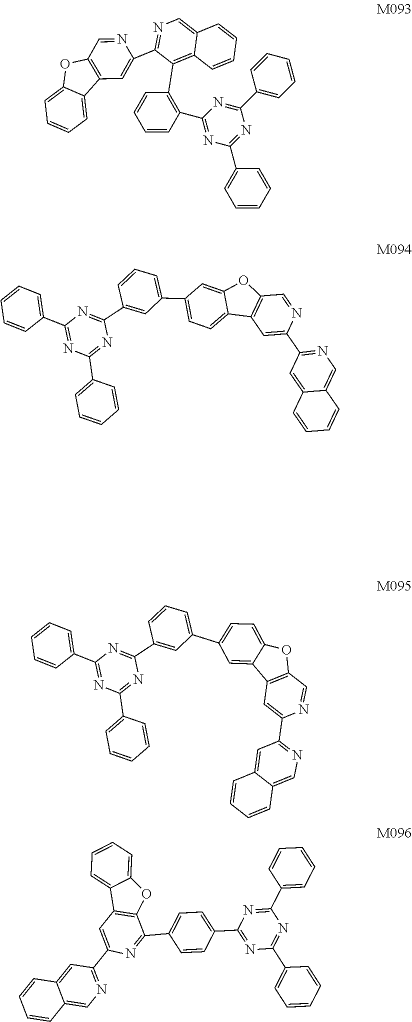

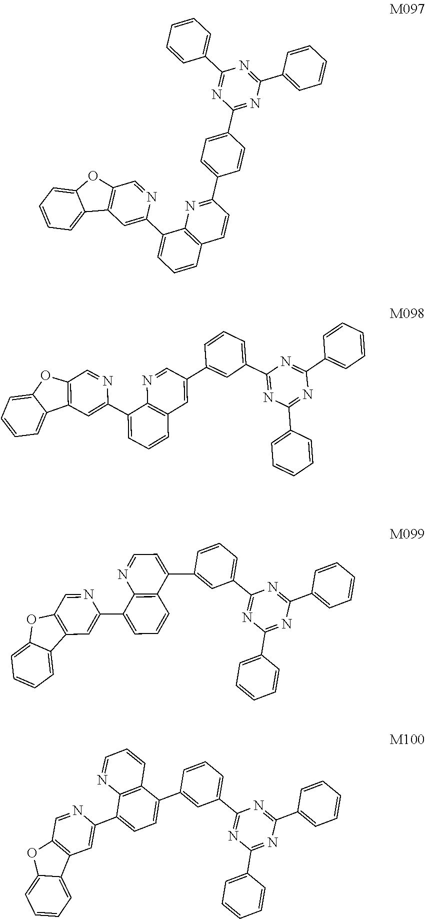 [SMILES: c1ccc(-c2nc(-c3ccccc3)nc(-c3ccc(-c4ccc5cccc(-c6cc7c(cn6)oc6ccccc67)c5n4)cc3)n2)cc1 ; c1ccc(-c2nc(-c3ccccc3)nc(-c3cccc(-c4cnc5c(-c6cc7c(cn6)oc6ccccc67)cccc5c4)c3)n2)cc1 ; c1ccc(-c2nc(-c3ccccc3)nc(-c3cccc(-c4ccnc5c(-c6cc7c(cn6)oc6ccccc67)cccc45)c3)n2)cc1 ; c1ccc(-c2nc(-c3ccccc3)nc(-c3cccc(-c4ccc(-c5cc6c(cn5)oc5ccccc56)c5ncccc45)c3)n2)cc1]

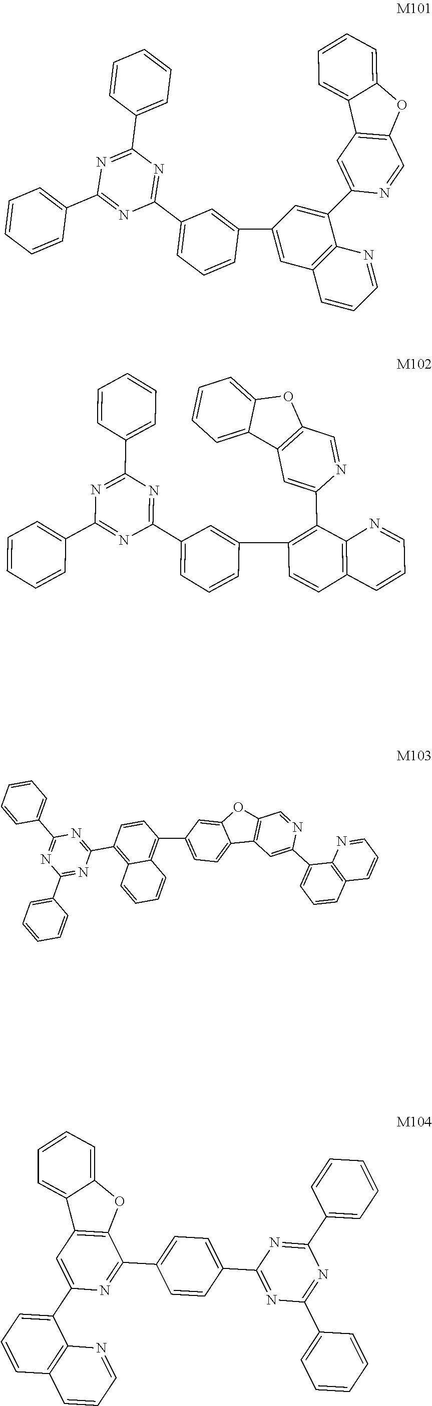 [SMILES: c1ccc(-c2nc(-c3ccccc3)nc(-c3cccc(-c4cc(-c5cc6c(cn5)oc5ccccc56)c5ncccc5c4)c3)n2)cc1 ; c1ccc(-c2nc(-c3ccccc3)nc(-c3cccc(-c4ccc5cccnc5c4-c4cc5c(cn4)oc4ccccc45)c3)n2)cc1 ; c1ccc(-c2nc(-c3ccccc3)nc(-c3ccc(-c4ccc5c(c4)oc4cnc(-c6cccc7cccnc67)cc45)c4ccccc34)n2)cc1 ; c1ccc(-c2nc(-c3ccccc3)nc(-c3ccc(-c4nc(-c5cccc6cccnc56)cc5c4oc4ccccc45)cc3)n2)cc1]

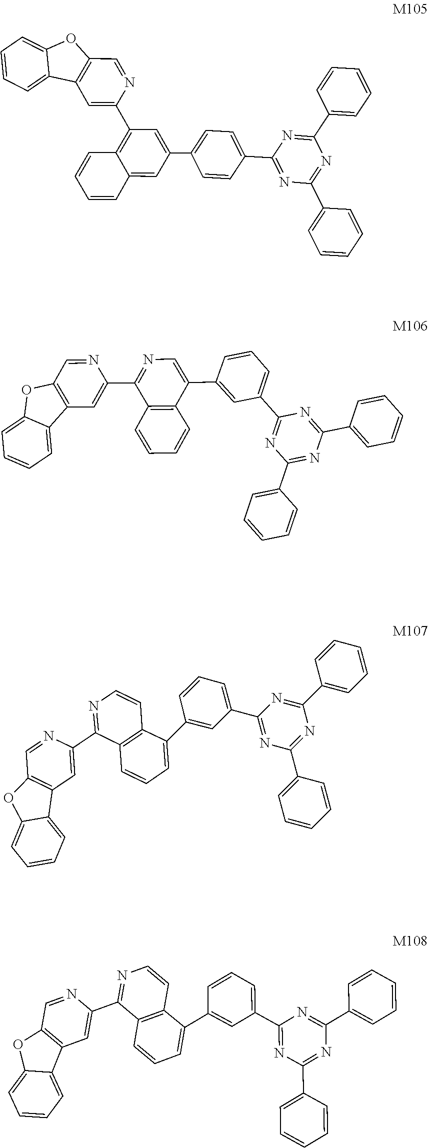 [SMILES: c1ccc(-c2nc(-c3ccccc3)nc(-c3ccc(-c4cc(-c5cc6c(cn5)oc5ccccc56)c5ccccc5c4)cc3)n2)cc1 ; c1ccc(-c2nc(-c3ccccc3)nc(-c3cccc(-c4cnc(-c5cc6c(cn5)oc5ccccc56)c5ccccc45)c3)n2)cc1 ; c1ccc(-c2nc(-c3ccccc3)nc(-c3cccc(-c4cccc5c(-c6cc7c(cn6)oc6ccccc67)nccc45)c3)n2)cc1 ; c1ccc(-c2nc(-c3ccccc3)nc(-c3cccc(-c4cccc5c(-c6cc7c(cn6)oc6ccccc67)nccc45)c3)n2)cc1]

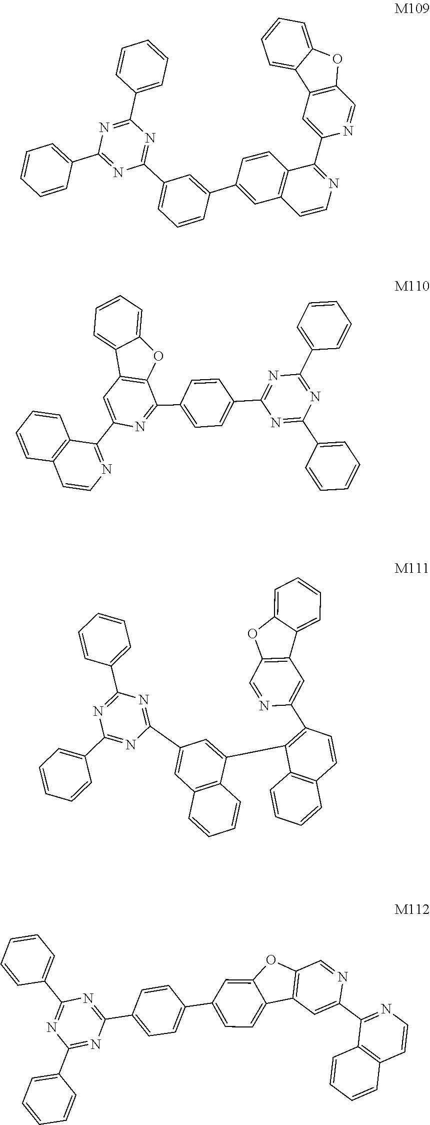 [SMILES: c1ccc(-c2nc(-c3ccccc3)nc(-c3cccc(-c4ccc5c(-c6cc7c(cn6)oc6ccccc67)nccc5c4)c3)n2)cc1 ; c1ccc(-c2nc(-c3ccccc3)nc(-c3ccc(-c4nc(-c5nccc6ccccc56)cc5c4oc4ccccc45)cc3)n2)cc1 ; c1ccc(-c2nc(-c3ccccc3)nc(-c3cc(-c4c(-c5cc6c(cn5)oc5ccccc56)ccc5ccccc45)c4ccccc4c3)n2)cc1 ; c1ccc(-c2nc(-c3ccccc3)nc(-c3ccc(-c4ccc5c(c4)oc4cnc(-c6nccc7ccccc67)cc45)cc3)n2)cc1]

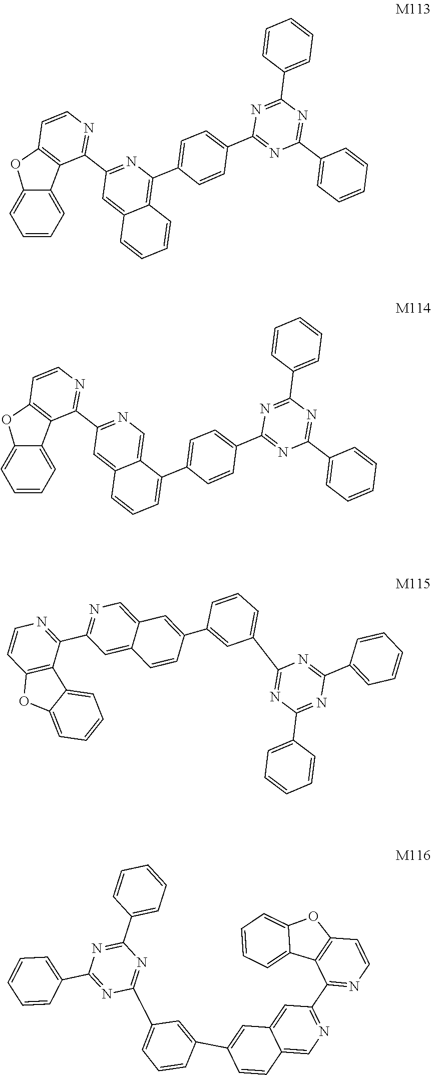 [SMILES: c1ccc(-c2nc(-c3ccccc3)nc(-c3ccc(-c4nc(-c5nccc6oc7ccccc7c56)cc5ccccc45)cc3)n2)cc1 ; c1ccc(-c2nc(-c3ccccc3)nc(-c3ccc(-c4cccc5cc(-c6nccc7oc8ccccc8c67)ncc45)cc3)n2)cc1 ; c1ccc(-c2nc(-c3ccccc3)nc(-c3cccc(-c4ccc5cc(-c6nccc7oc8ccccc8c67)ncc5c4)c3)n2)cc1 ; c1ccc(-c2nc(-c3ccccc3)nc(-c3cccc(-c4ccc5cnc(-c6nccc7oc8ccccc8c67)cc5c4)c3)n2)cc1]

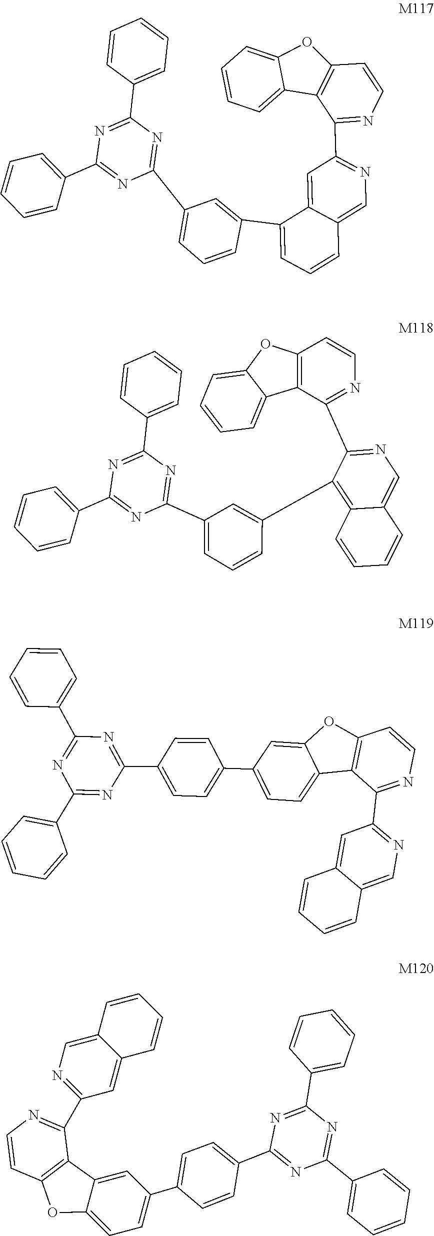 [SMILES: c1ccc(-c2nc(-c3ccccc3)nc(-c3cccc(-c4cccc5cnc(-c6nccc7oc8ccccc8c67)cc45)c3)n2)cc1 ; c1ccc(-c2nc(-c3ccccc3)nc(-c3cccc(-c4c(-c5nccc6oc7ccccc7c56)ncc5ccccc45)c3)n2)cc1 ; c1ccc(-c2nc(-c3ccccc3)nc(-c3ccc(-c4ccc5c(c4)oc4ccnc(-c6cc7ccccc7cn6)c45)cc3)n2)cc1 ; c1ccc(-c2nc(-c3ccccc3)nc(-c3ccc(-c4ccc5oc6ccnc(-c7cc8ccccc8cn7)c6c5c4)cc3)n2)cc1]

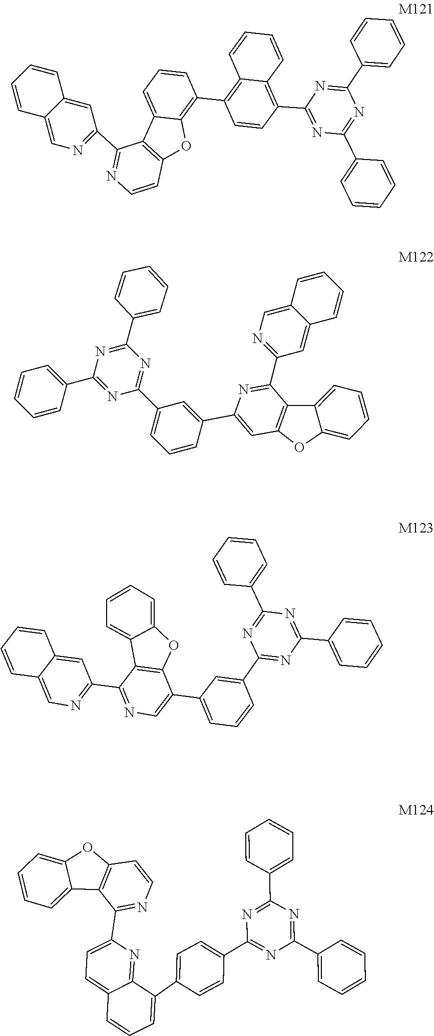 [SMILES: c1ccc(-c2nc(-c3ccccc3)nc(-c3ccc(-c4cccc5c4oc4ccnc(-c6cc7ccccc7cn6)c45)c4ccccc34)n2)cc1 ; c1ccc(-c2nc(-c3ccccc3)nc(-c3cccc(-c4cc5oc6ccccc6c5c(-c5cc6ccccc6cn5)n4)c3)n2)cc1 ; c1ccc(-c2nc(-c3ccccc3)nc(-c3cccc(-c4cnc(-c5cc6ccccc6cn5)c5c4oc4ccccc45)c3)n2)cc1 ; c1ccc(-c2nc(-c3ccccc3)nc(-c3ccc(-c4cccc5ccc(-c6nccc7oc8ccccc8c67)nc45)cc3)n2)cc1]

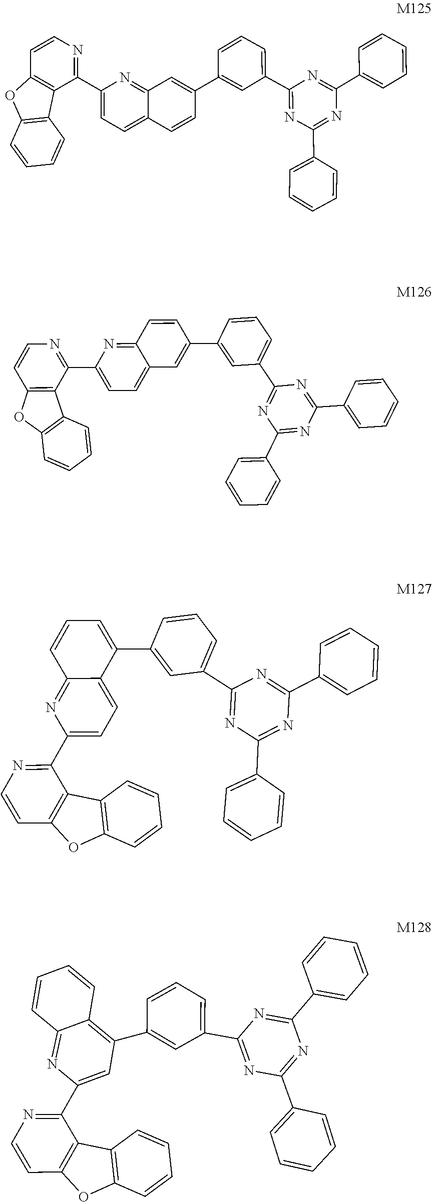 [SMILES: c1ccc(-c2nc(-c3ccccc3)nc(-c3cccc(-c4ccc5ccc(-c6nccc7oc8ccccc8c67)nc5c4)c3)n2)cc1 ; c1ccc(-c2nc(-c3ccccc3)nc(-c3cccc(-c4ccc5nc(-c6nccc7oc8ccccc8c67)ccc5c4)c3)n2)cc1 ; c1ccc(-c2nc(-c3ccccc3)nc(-c3cccc(-c4cccc5nc(-c6nccc7oc8ccccc8c67)ccc45)c3)n2)cc1 ; c1ccc(-c2nc(-c3ccccc3)nc(-c3cccc(-c4cc(-c5nccc6oc7ccccc7c56)nc5ccccc45)c3)n2)cc1]

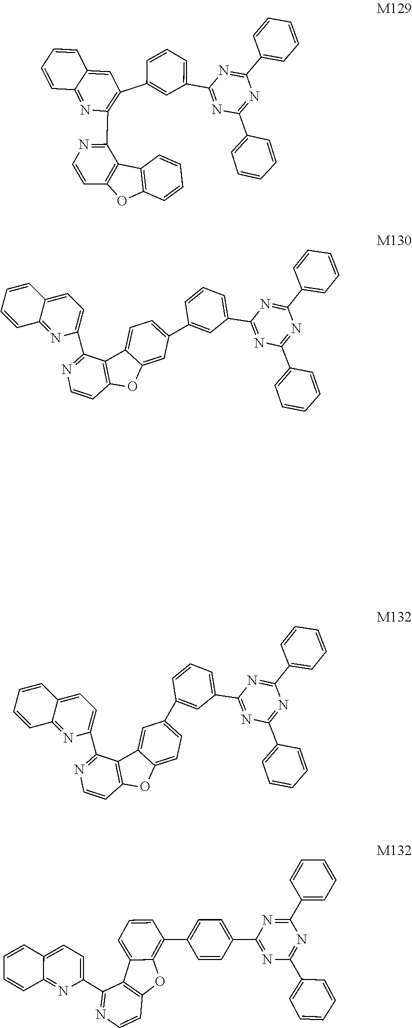 [SMILES: c1ccc(-c2nc(-c3ccccc3)nc(-c3cccc(-c4cc5ccccc5nc4-c4nccc5oc6ccccc6c45)c3)n2)cc1 ; c1ccc(-c2nc(-c3ccccc3)nc(-c3cccc(-c4ccc5c(c4)oc4ccnc(-c6ccc7ccccc7n6)c45)c3)n2)cc1 ; c1ccc(-c2nc(-c3ccccc3)nc(-c3cccc(-c4ccc5oc6ccnc(-c7ccc8ccccc8n7)c6c5c4)c3)n2)cc1 ; c1ccc(-c2nc(-c3ccccc3)nc(-c3ccc(-c4cccc5c4oc4ccnc(-c6ccc7ccccc7n6)c45)cc3)n2)cc1]

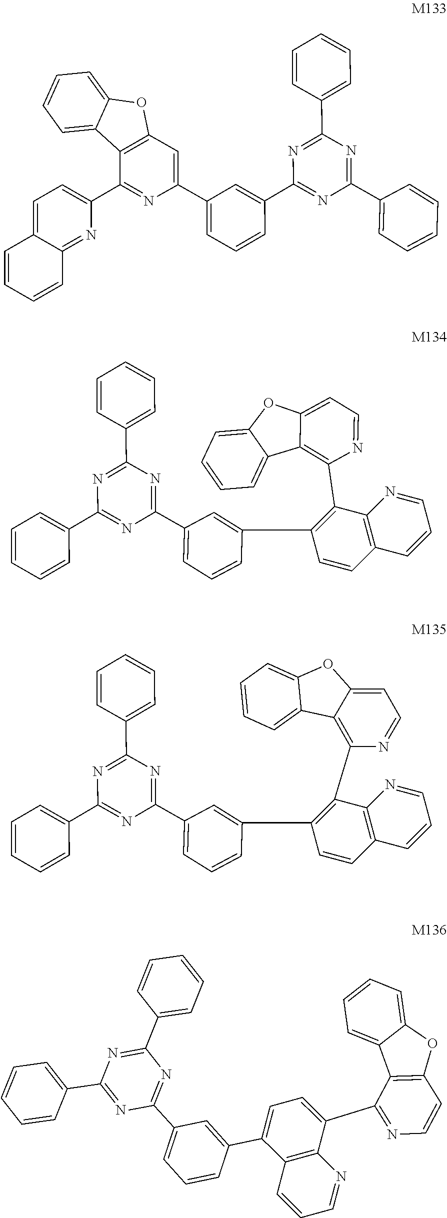 [SMILES: c1ccc(-c2nc(-c3ccccc3)nc(-c3cccc(-c4cc5oc6ccccc6c5c(-c5ccc6ccccc6n5)n4)c3)n2)cc1 ; c1ccc(-c2nc(-c3ccccc3)nc(-c3cccc(-c4ccc5cccnc5c4-c4nccc5oc6ccccc6c45)c3)n2)cc1 ; c1ccc(-c2nc(-c3ccccc3)nc(-c3cccc(-c4ccc5cccnc5c4-c4nccc5oc6ccccc6c45)c3)n2)cc1 ; c1ccc(-c2nc(-c3ccccc3)nc(-c3cccc(-c4ccc(-c5nccc6oc7ccccc7c56)c5ncccc45)c3)n2)cc1]

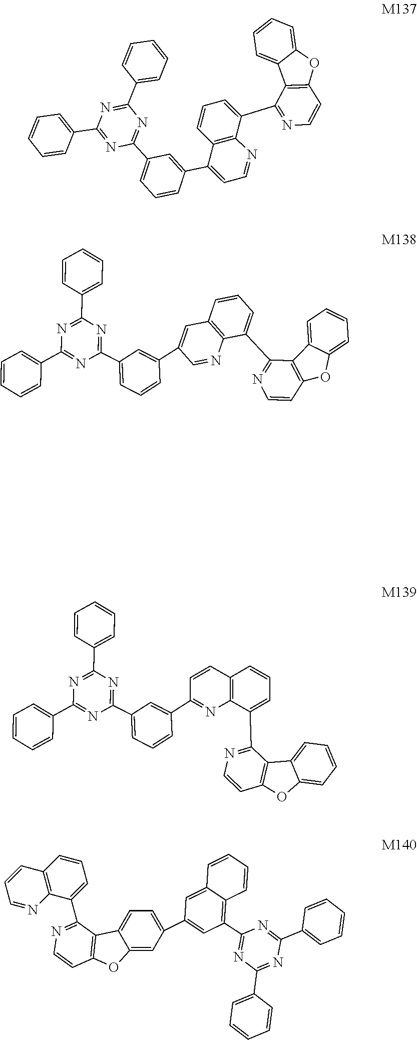 [SMILES: c1ccc(-c2nc(-c3ccccc3)nc(-c3cccc(-c4ccnc5c(-c6nccc7oc8ccccc8c67)cccc45)c3)n2)cc1 ; c1ccc(-c2nc(-c3ccccc3)nc(-c3cccc(-c4cnc5c(-c6nccc7oc8ccccc8c67)cccc5c4)c3)n2)cc1 ; c1ccc(-c2nc(-c3ccccc3)nc(-c3cccc(-c4ccc5cccc(-c6nccc7oc8ccccc8c67)c5n4)c3)n2)cc1 ; c1ccc(-c2nc(-c3ccccc3)nc(-c3cc(-c4ccc5c(c4)oc4ccnc(-c6cccc7cccnc67)c45)cc4ccccc34)n2)cc1]

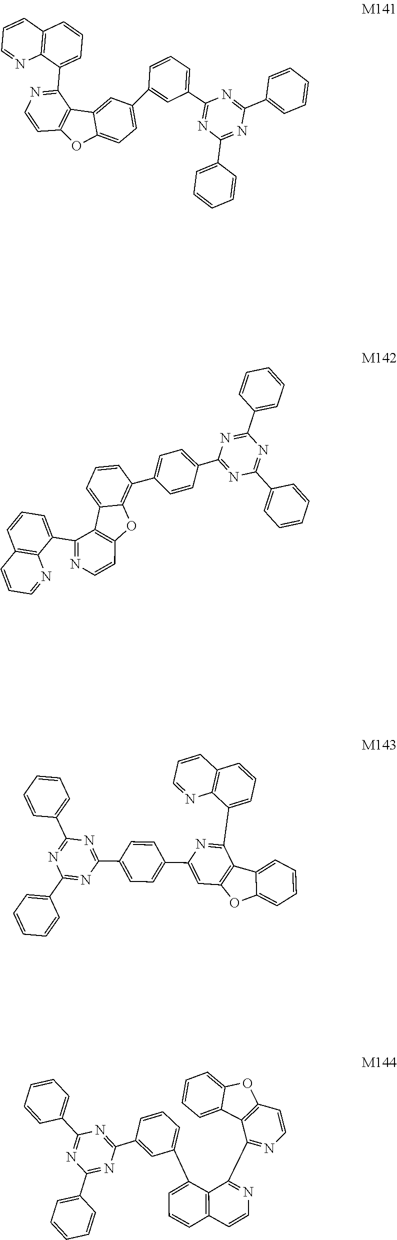 [SMILES: c1ccc(-c2nc(-c3ccccc3)nc(-c3cccc(-c4ccc5oc6ccnc(-c7cccc8cccnc78)c6c5c4)c3)n2)cc1 ; c1ccc(-c2nc(-c3ccccc3)nc(-c3ccc(-c4cccc5c4oc4ccnc(-c6cccc7cccnc67)c45)cc3)n2)cc1 ; c1ccc(-c2nc(-c3ccccc3)nc(-c3ccc(-c4cc5oc6ccccc6c5c(-c5cccc6cccnc56)n4)cc3)n2)cc1 ; c1ccc(-c2nc(-c3ccccc3)nc(-c3cccc(-c4cccc5ccnc(-c6nccc7oc8ccccc8c67)c45)c3)n2)cc1]

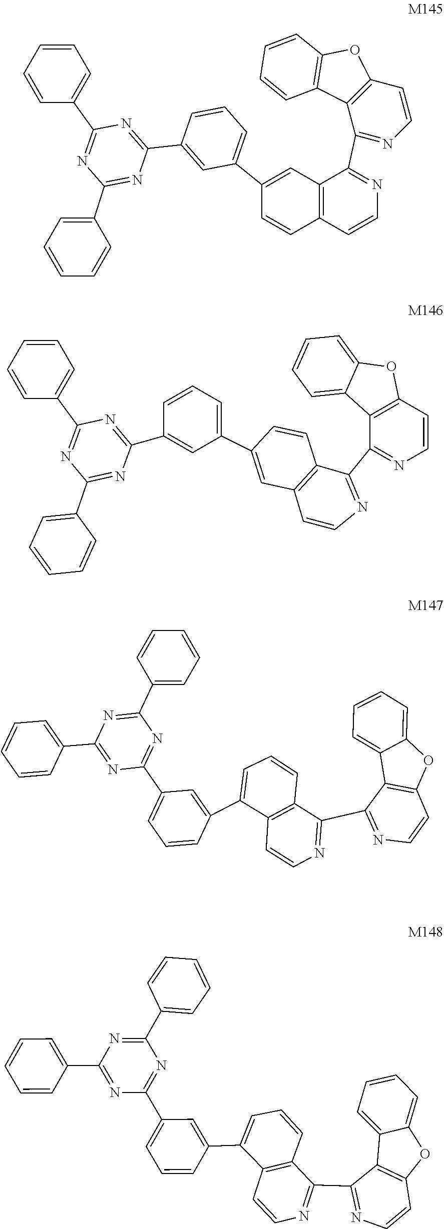 [SMILES: c1ccc(-c2nc(-c3ccccc3)nc(-c3cccc(-c4ccc5ccnc(-c6nccc7oc8ccccc8c67)c5c4)c3)n2)cc1 ; c1ccc(-c2nc(-c3ccccc3)nc(-c3cccc(-c4ccc5c(-c6nccc7oc8ccccc8c67)nccc5c4)c3)n2)cc1 ; c1ccc(-c2nc(-c3ccccc3)nc(-c3cccc(-c4cccc5c(-c6nccc7oc8ccccc8c67)nccc45)c3)n2)cc1 ; c1ccc(-c2nc(-c3ccccc3)nc(-c3cccc(-c4cccc5c(-c6nccc7oc8ccccc8c67)nccc45)c3)n2)cc1]

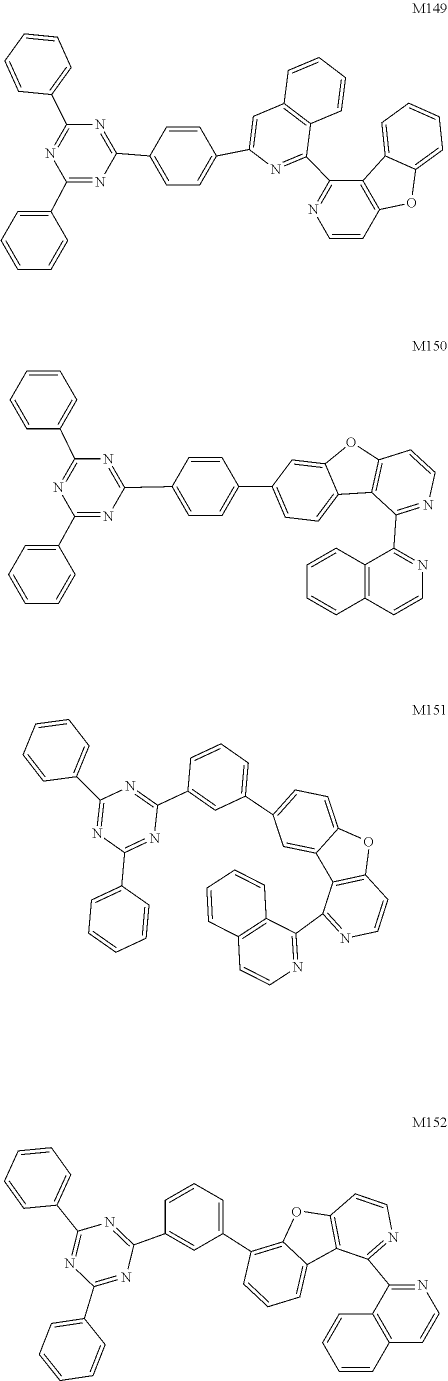 [SMILES: c1ccc(-c2nc(-c3ccccc3)nc(-c3ccc(-c4cc5ccccc5c(-c5nccc6oc7ccccc7c56)n4)cc3)n2)cc1 ; c1ccc(-c2nc(-c3ccccc3)nc(-c3ccc(-c4ccc5c(c4)oc4ccnc(-c6nccc7ccccc67)c45)cc3)n2)cc1 ; c1ccc(-c2nc(-c3ccccc3)nc(-c3cccc(-c4ccc5oc6ccnc(-c7nccc8ccccc78)c6c5c4)c3)n2)cc1 ; c1ccc(-c2nc(-c3ccccc3)nc(-c3cccc(-c4cccc5c4oc4ccnc(-c6nccc7ccccc67)c45)c3)n2)cc1]

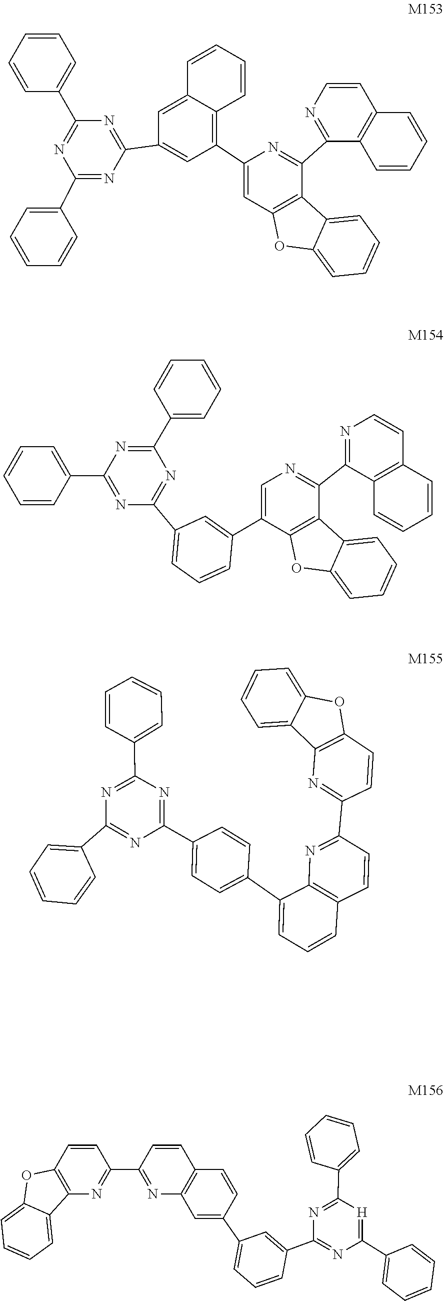

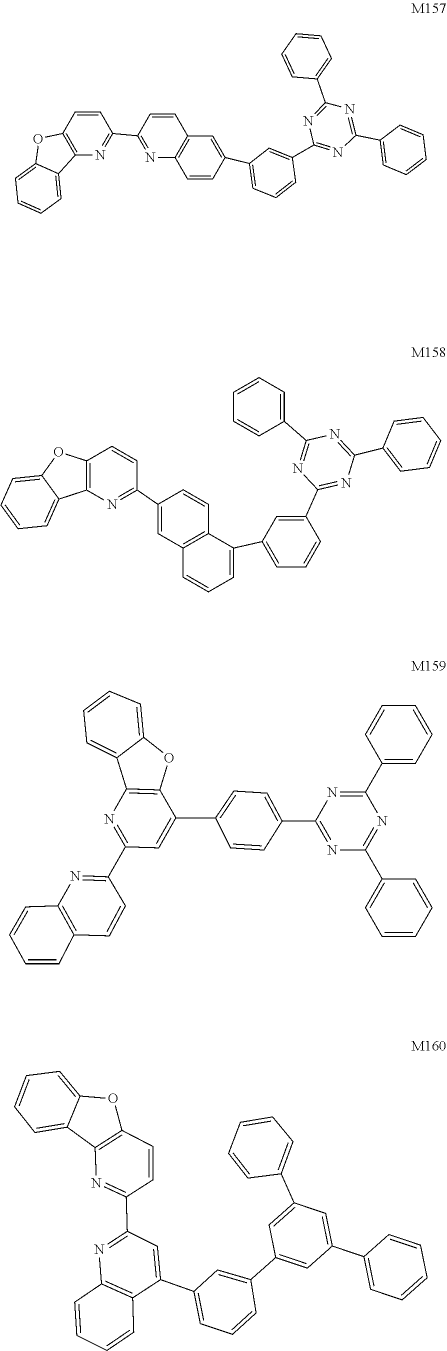 [SMILES: c1ccc(-c2nc(-c3ccccc3)nc(-c3cccc(-c4ccc5nc(-c6ccc7oc8ccccc8c7n6)ccc5c4)c3)n2)cc1 ; c1ccc(-c2nc(-c3ccccc3)nc(-c3cccc(-c4cccc5cc(-c6ccc7oc8ccccc8c7n6)ccc45)c3)n2)cc1 ; c1ccc(-c2nc(-c3ccccc3)nc(-c3ccc(-c4cc(-c5ccc6ccccc6n5)nc5c4oc4ccccc45)cc3)n2)cc1 ; c1ccc(-c2cc(-c3ccccc3)cc(-c3cccc(-c4cc(-c5ccc6oc7ccccc7c6n5)nc5ccccc45)c3)c2)cc1]

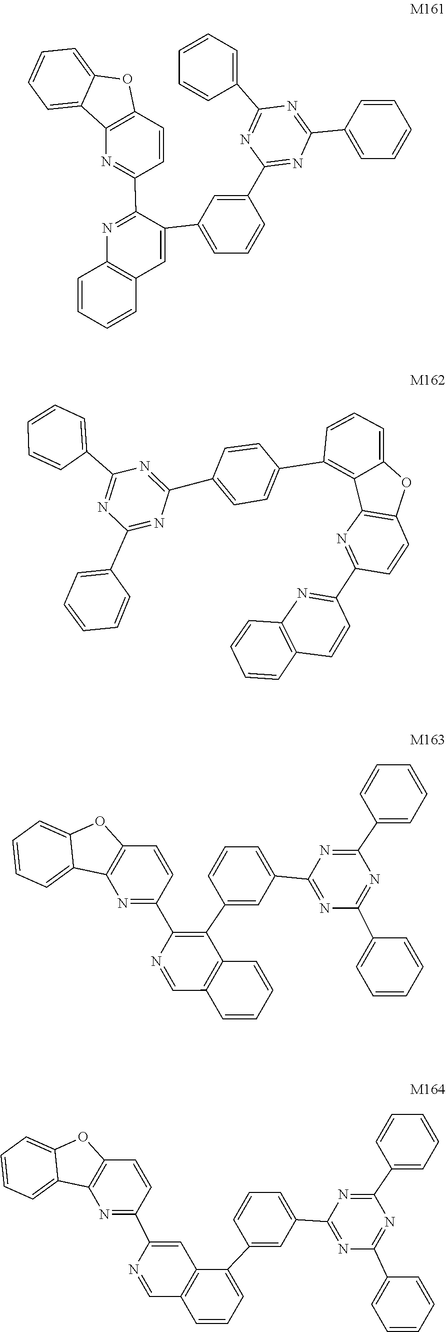 [SMILES: c1ccc(-c2nc(-c3ccccc3)nc(-c3cccc(-c4cc5ccccc5nc4-c4ccc5oc6ccccc6c5n4)c3)n2)cc1 ; c1ccc(-c2nc(-c3ccccc3)nc(-c3ccc(-c4cccc5oc6ccc(-c7ccc8ccccc8n7)nc6c45)cc3)n2)cc1 ; c1ccc(-c2nc(-c3ccccc3)nc(-c3cccc(-c4c(-c5ccc6oc7ccccc7c6n5)ncc5ccccc45)c3)n2)cc1 ; c1ccc(-c2nc(-c3ccccc3)nc(-c3cccc(-c4cccc5cnc(-c6ccc7oc8ccccc8c7n6)cc45)c3)n2)cc1]

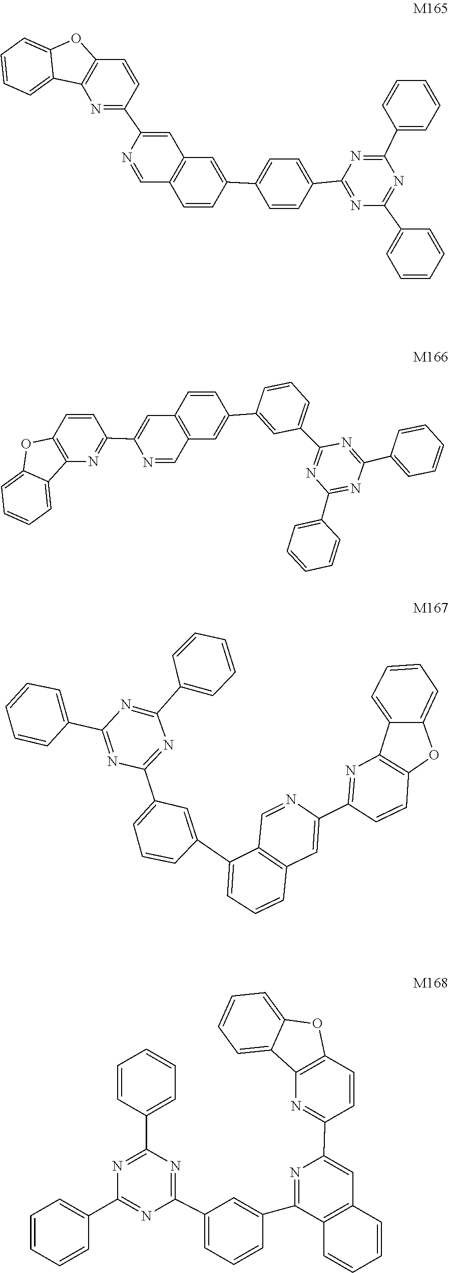 [SMILES: c1ccc(-c2nc(-c3ccccc3)nc(-c3ccc(-c4ccc5cnc(-c6ccc7oc8ccccc8c7n6)cc5c4)cc3)n2)cc1 ; c1ccc(-c2nc(-c3ccccc3)nc(-c3cccc(-c4ccc5cc(-c6ccc7oc8ccccc8c7n6)ncc5c4)c3)n2)cc1 ; c1ccc(-c2nc(-c3ccccc3)nc(-c3cccc(-c4cccc5cc(-c6ccc7oc8ccccc8c7n6)ncc45)c3)n2)cc1 ; c1ccc(-c2nc(-c3ccccc3)nc(-c3cccc(-c4nc(-c5ccc6oc7ccccc7c6n5)cc5ccccc45)c3)n2)cc1]

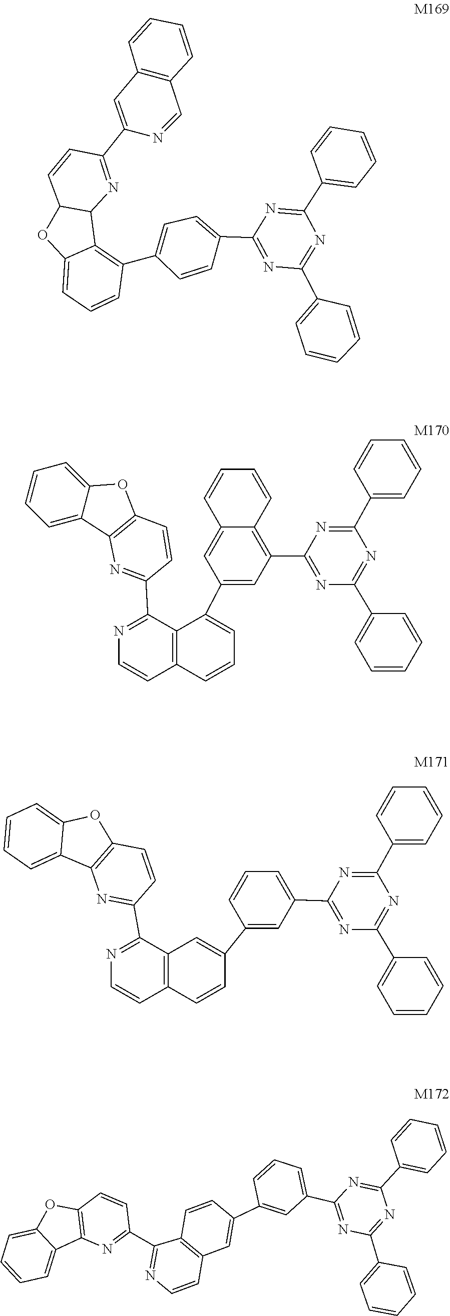 [SMILES: C1=CC2Oc3cccc(-c4ccc(-c5nc(-c6ccccc6)nc(-c6ccccc6)n5)cc4)c3C2N=C1c1cc2ccccc2cn1 ; c1ccc(-c2nc(-c3ccccc3)nc(-c3cc(-c4cccc5ccnc(-c6ccc7oc8ccccc8c7n6)c45)cc4ccccc34)n2)cc1 ; c1ccc(-c2nc(-c3ccccc3)nc(-c3cccc(-c4ccc5ccnc(-c6ccc7oc8ccccc8c7n6)c5c4)c3)n2)cc1 ; c1ccc(-c2nc(-c3ccccc3)nc(-c3cccc(-c4ccc5c(-c6ccc7oc8ccccc8c7n6)nccc5c4)c3)n2)cc1]

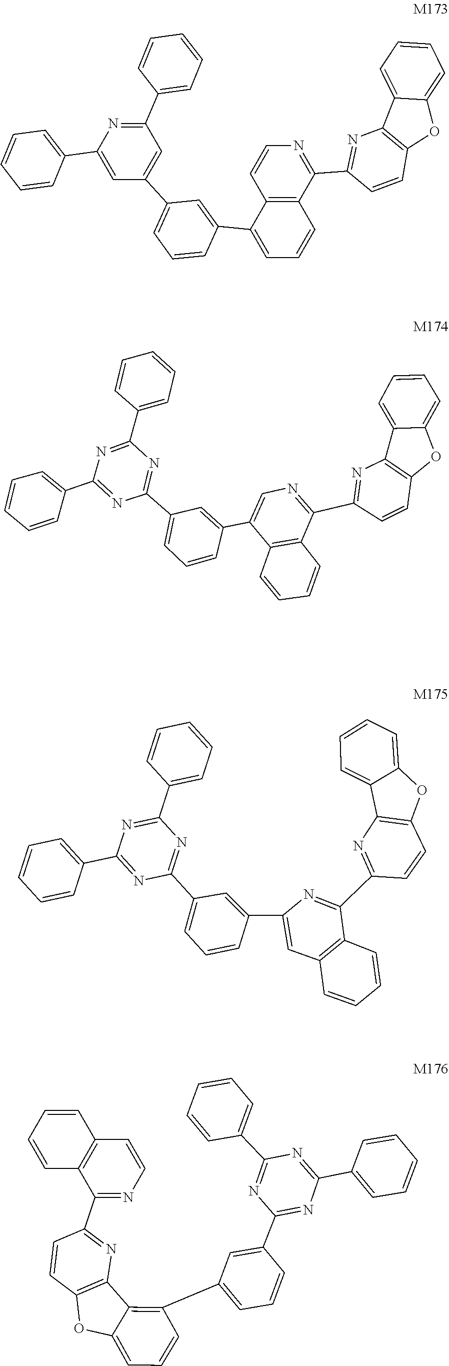 [SMILES: c1ccc(-c2cc(-c3cccc(-c4cccc5c(-c6ccc7oc8ccccc8c7n6)nccc45)c3)cc(-c3ccccc3)n2)cc1 ; c1ccc(-c2nc(-c3ccccc3)nc(-c3cccc(-c4cnc(-c5ccc6oc7ccccc7c6n5)c5ccccc45)c3)n2)cc1 ; c1ccc(-c2nc(-c3ccccc3)nc(-c3cccc(-c4cc5ccccc5c(-c5ccc6oc7ccccc7c6n5)n4)c3)n2)cc1 ; c1ccc(-c2nc(-c3ccccc3)nc(-c3cccc(-c4cccc5oc6ccc(-c7nccc8ccccc78)nc6c45)c3)n2)cc1]

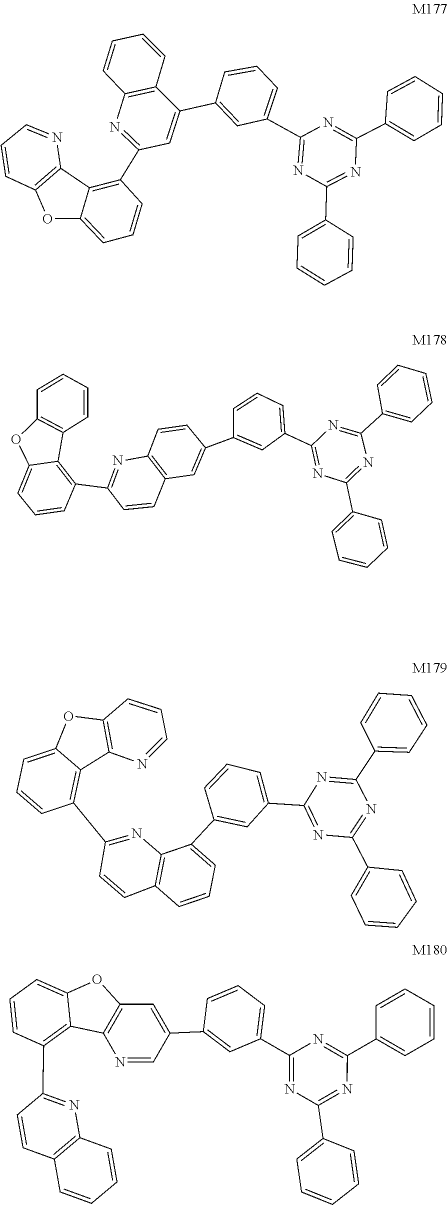 [SMILES: c1ccc(-c2nc(-c3ccccc3)nc(-c3cccc(-c4cc(-c5cccc6oc7cccnc7c56)nc5ccccc45)c3)n2)cc1 ; c1ccc(-c2nc(-c3ccccc3)nc(-c3cccc(-c4ccc5nc(-c6cccc7oc8ccccc8c67)ccc5c4)c3)n2)cc1 ; c1ccc(-c2nc(-c3ccccc3)nc(-c3cccc(-c4cccc5ccc(-c6cccc7oc8cccnc8c67)nc45)c3)n2)cc1 ; c1ccc(-c2nc(-c3ccccc3)nc(-c3cccc(-c4cnc5c(c4)oc4cccc(-c6ccc7ccccc7n6)c45)c3)n2)cc1]

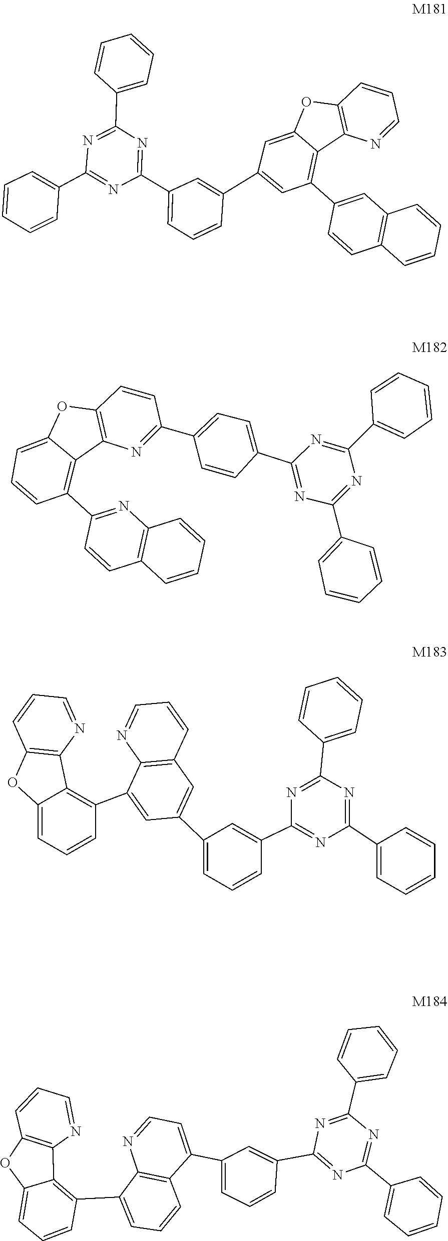 [SMILES: c1ccc(-c2nc(-c3ccccc3)nc(-c3cccc(-c4cc(-c5ccc6ccccc6c5)c5c(c4)oc4cccnc45)c3)n2)cc1 ; c1ccc(-c2nc(-c3ccccc3)nc(-c3ccc(-c4ccc5oc6cccc(-c7ccc8ccccc8n7)c6c5n4)cc3)n2)cc1 ; c1ccc(-c2nc(-c3ccccc3)nc(-c3cccc(-c4cc(-c5cccc6oc7cccnc7c56)c5ncccc5c4)c3)n2)cc1 ; c1ccc(-c2nc(-c3ccccc3)nc(-c3cccc(-c4ccnc5c(-c6cccc7oc8cccnc8c67)cccc45)c3)n2)cc1]

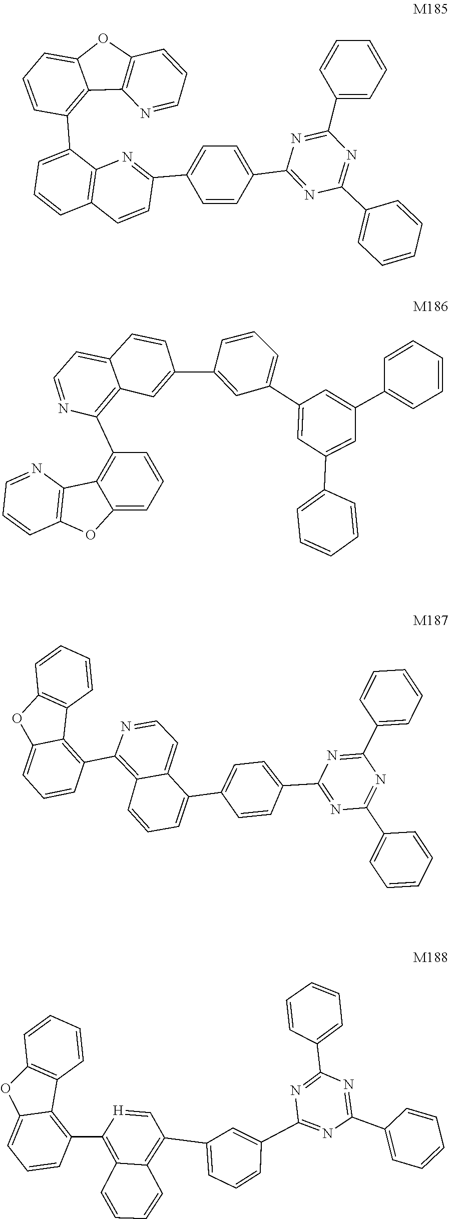

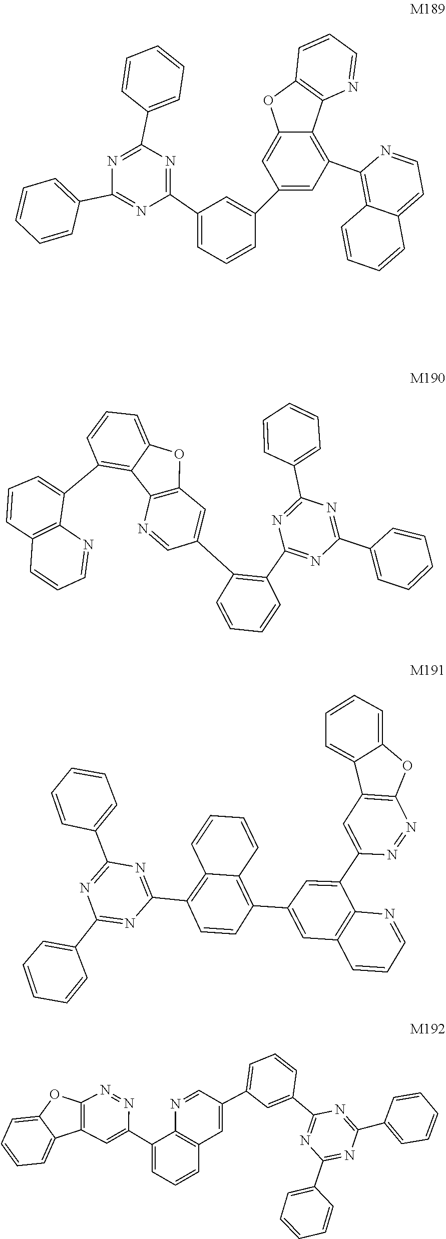 [SMILES: c1ccc(-c2nc(-c3ccccc3)nc(-c3cccc(-c4cc(-c5nccc6ccccc56)c5c(c4)oc4cccnc45)c3)n2)cc1 ; c1ccc(-c2nc(-c3ccccc3)nc(-c3ccccc3-c3cnc4c(c3)oc3cccc(-c5cccc6cccnc56)c34)n2)cc1 ; c1ccc(-c2nc(-c3ccccc3)nc(-c3ccc(-c4cc(-c5cc6c(nn5)oc5ccccc56)c5ncccc5c4)c4ccccc34)n2)cc1 ; c1ccc(-c2nc(-c3ccccc3)nc(-c3cccc(-c4cnc5c(-c6cc7c(nn6)oc6ccccc67)cccc5c4)c3)n2)cc1]

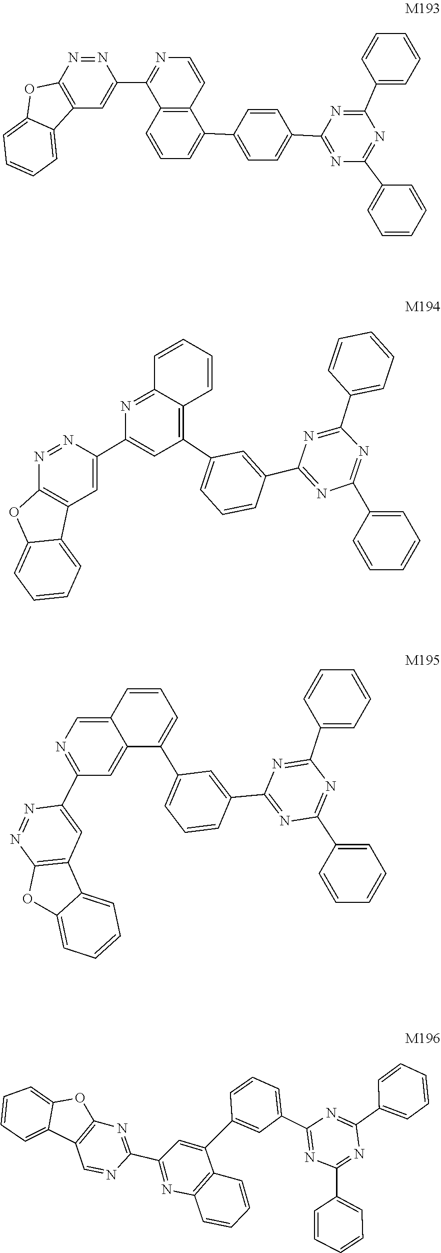 [SMILES: c1ccc(-c2nc(-c3ccccc3)nc(-c3ccc(-c4cccc5c(-c6cc7c(nn6)oc6ccccc67)nccc45)cc3)n2)cc1 ; c1ccc(-c2nc(-c3ccccc3)nc(-c3cccc(-c4cc(-c5cc6c(nn5)oc5ccccc56)nc5ccccc45)c3)n2)cc1 ; c1ccc(-c2nc(-c3ccccc3)nc(-c3cccc(-c4cccc5cnc(-c6cc7c(nn6)oc6ccccc67)cc45)c3)n2)cc1 ; c1ccc(-c2nc(-c3ccccc3)nc(-c3cccc(-c4cc(-c5ncc6c(n5)oc5ccccc56)nc5ccccc45)c3)n2)cc1]

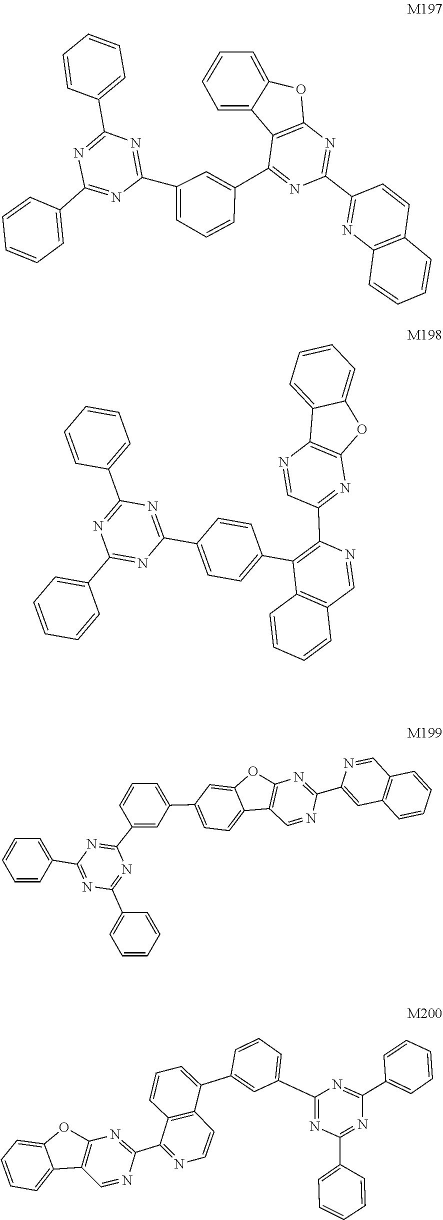 [SMILES: c1ccc(-c2nc(-c3ccccc3)nc(-c3cccc(-c4nc(-c5ccc6ccccc6n5)nc5oc6ccccc6c45)c3)n2)cc1 ; c1ccc(-c2nc(-c3ccccc3)nc(-c3ccc(-c4c(-c5cnc6c(n5)oc5ccccc56)ncc5ccccc45)cc3)n2)cc1 ; c1ccc(-c2nc(-c3ccccc3)nc(-c3cccc(-c4ccc5c(c4)oc4nc(-c6cc7ccccc7cn6)ncc45)c3)n2)cc1 ; c1ccc(-c2nc(-c3ccccc3)nc(-c3cccc(-c4cccc5c(-c6ncc7c(n6)oc6ccccc67)nccc45)c3)n2)cc1]

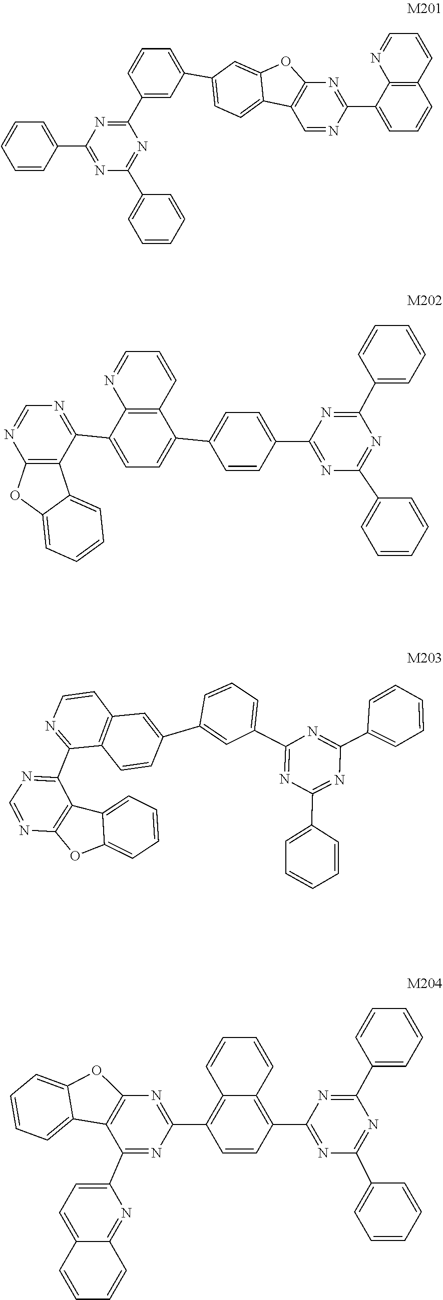 [SMILES: c1ccc(-c2nc(-c3ccccc3)nc(-c3cccc(-c4ccc5c(c4)oc4nc(-c6cccc7cccnc67)ncc45)c3)n2)cc1 ; c1ccc(-c2nc(-c3ccccc3)nc(-c3ccc(-c4ccc(-c5ncnc6oc7ccccc7c56)c5ncccc45)cc3)n2)cc1 ; c1ccc(-c2nc(-c3ccccc3)nc(-c3cccc(-c4ccc5c(-c6ncnc7oc8ccccc8c67)nccc5c4)c3)n2)cc1 ; c1ccc(-c2nc(-c3ccccc3)nc(-c3ccc(-c4nc(-c5ccc6ccccc6n5)c5c(n4)oc4ccccc45)c4ccccc34)n2)cc1]

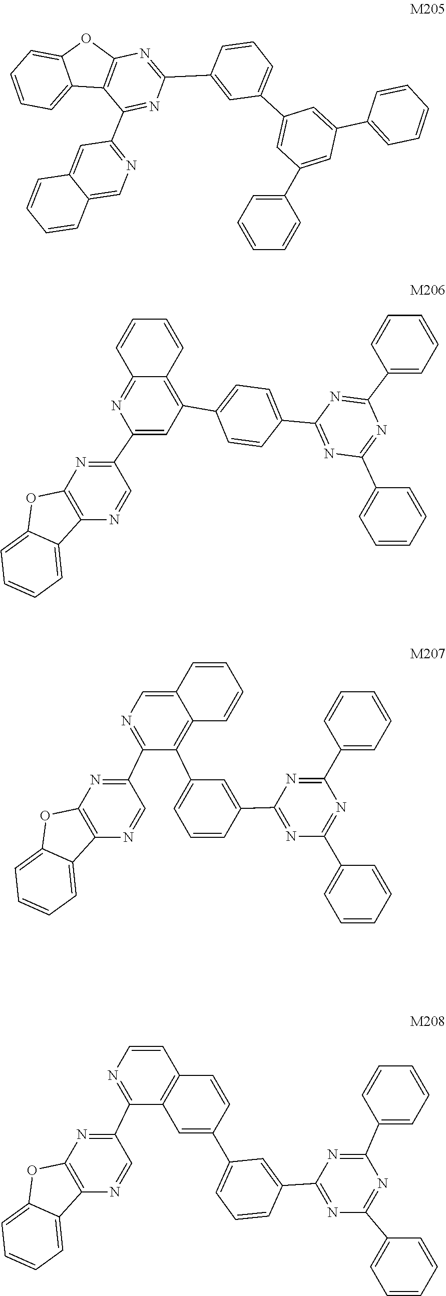 [SMILES: c1ccc(-c2cc(-c3ccccc3)cc(-c3cccc(-c4nc(-c5cc6ccccc6cn5)c5c(n4)oc4ccccc45)c3)c2)cc1 ; c1ccc(-c2nc(-c3ccccc3)nc(-c3ccc(-c4cc(-c5cnc6c(n5)oc5ccccc56)nc5ccccc45)cc3)n2)cc1 ; c1ccc(-c2nc(-c3ccccc3)nc(-c3cccc(-c4c(-c5cnc6c(n5)oc5ccccc56)ncc5ccccc45)c3)n2)cc1 ; c1ccc(-c2nc(-c3ccccc3)nc(-c3cccc(-c4ccc5ccnc(-c6cnc7c(n6)oc6ccccc67)c5c4)c3)n2)cc1]

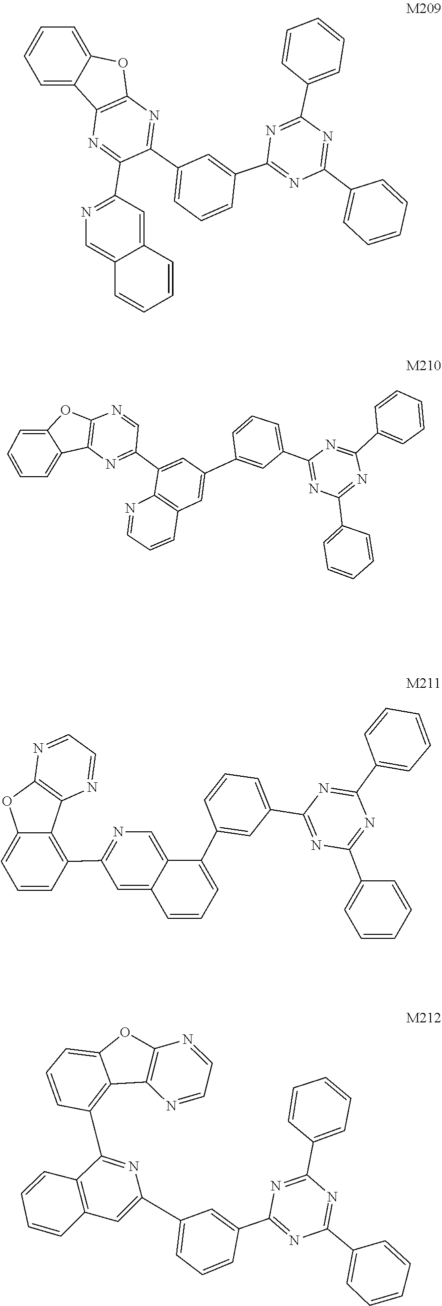 [SMILES: c1ccc(-c2nc(-c3ccccc3)nc(-c3cccc(-c4nc5oc6ccccc6c5nc4-c4cc5ccccc5cn4)c3)n2)cc1 ; c1ccc(-c2nc(-c3ccccc3)nc(-c3cccc(-c4cc(-c5cnc6oc7ccccc7c6n5)c5ncccc5c4)c3)n2)cc1 ; c1ccc(-c2nc(-c3ccccc3)nc(-c3cccc(-c4cccc5cc(-c6cccc7oc8nccnc8c67)ncc45)c3)n2)cc1 ; c1ccc(-c2nc(-c3ccccc3)nc(-c3cccc(-c4cc5ccccc5c(-c5cccc6oc7nccnc7c56)n4)c3)n2)cc1]

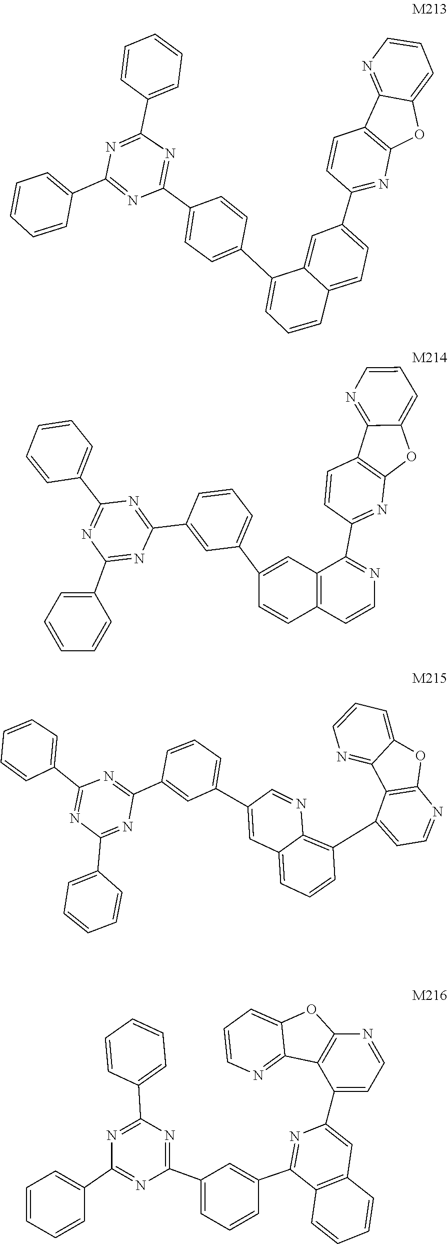

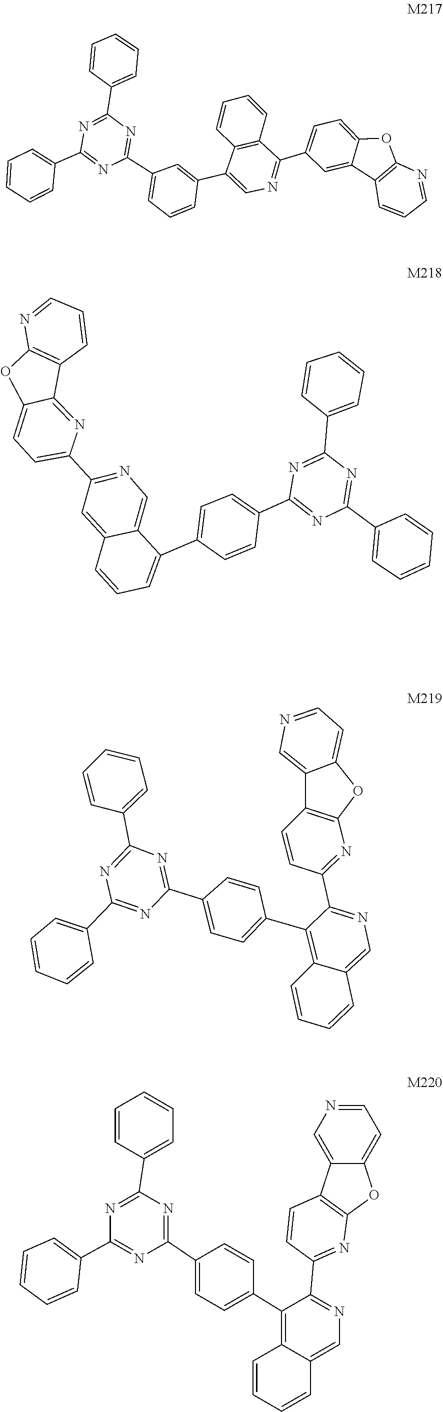 [SMILES: c1ccc(-c2nc(-c3ccccc3)nc(-c3cccc(-c4cnc(-c5ccc6oc7ncccc7c6c5)c5ccccc45)c3)n2)cc1 ; c1ccc(-c2nc(-c3ccccc3)nc(-c3ccc(-c4cccc5cc(-c6ccc7oc8ncccc8c7n6)ncc45)cc3)n2)cc1 ; c1ccc(-c2nc(-c3ccccc3)nc(-c3ccc(-c4c(-c5ccc6c(n5)oc5ccncc56)ncc5ccccc45)cc3)n2)cc1 ; c1ccc(-c2nc(-c3ccccc3)nc(-c3ccc(-c4c(-c5ccc6c(n5)oc5ccncc56)ncc5ccccc45)cc3)n2)cc1]

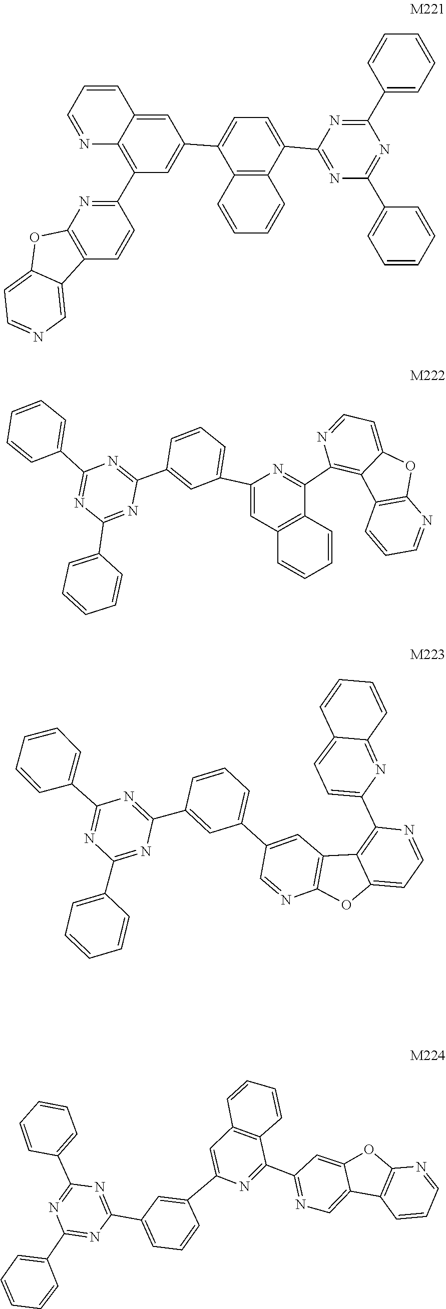 [SMILES: c1ccc(-c2nc(-c3ccccc3)nc(-c3ccc(-c4cc(-c5ccc6c(n5)oc5ccncc56)c5ncccc5c4)c4ccccc34)n2)cc1 ; c1ccc(-c2nc(-c3ccccc3)nc(-c3cccc(-c4cc5ccccc5c(-c5nccc6oc7ncccc7c56)n4)c3)n2)cc1 ; c1ccc(-c2nc(-c3ccccc3)nc(-c3cccc(-c4cnc5oc6ccnc(-c7ccc8ccccc8n7)c6c5c4)c3)n2)cc1 ; c1ccc(-c2nc(-c3ccccc3)nc(-c3cccc(-c4cc5ccccc5c(-c5cc6oc7ncccc7c6cn5)n4)c3)n2)cc1]

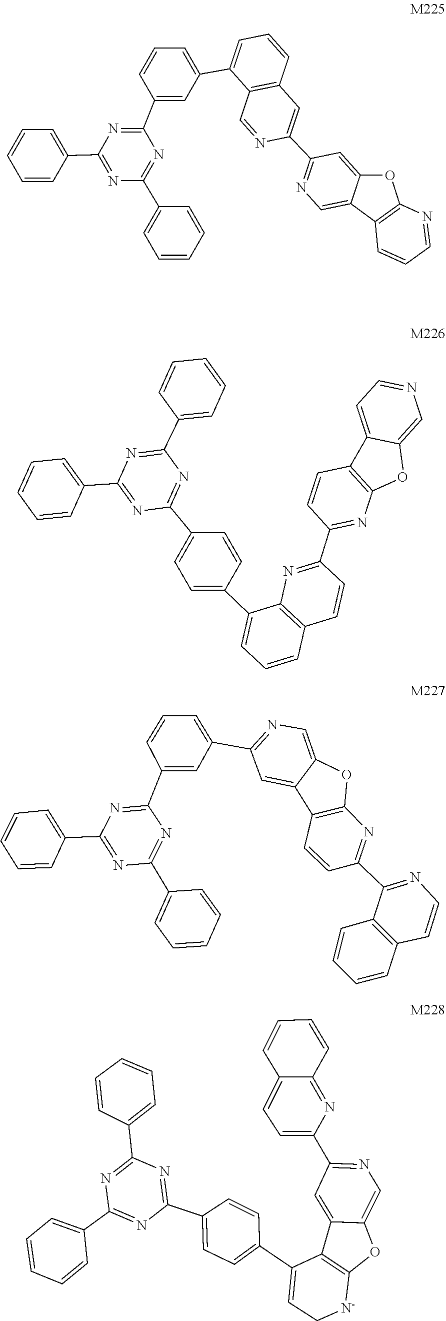 [SMILES: c1ccc(-c2nc(-c3ccccc3)nc(-c3cccc(-c4cccc5cc(-c6cc7oc8ncccc8c7cn6)ncc45)c3)n2)cc1 ; c1ccc(-c2nc(-c3ccccc3)nc(-c3ccc(-c4cccc5ccc(-c6ccc7c(n6)oc6cnccc67)nc45)cc3)n2)cc1 ; c1ccc(-c2nc(-c3ccccc3)nc(-c3cccc(-c4cc5c(cn4)oc4nc(-c6nccc7ccccc67)ccc45)c3)n2)cc1 ; C1=C(c2ccc(-c3nc(-c4ccccc4)nc(-c4ccccc4)n3)cc2)c2c(oc3cnc(-c4ccc5ccccc5n4)cc23)[N-]C1]

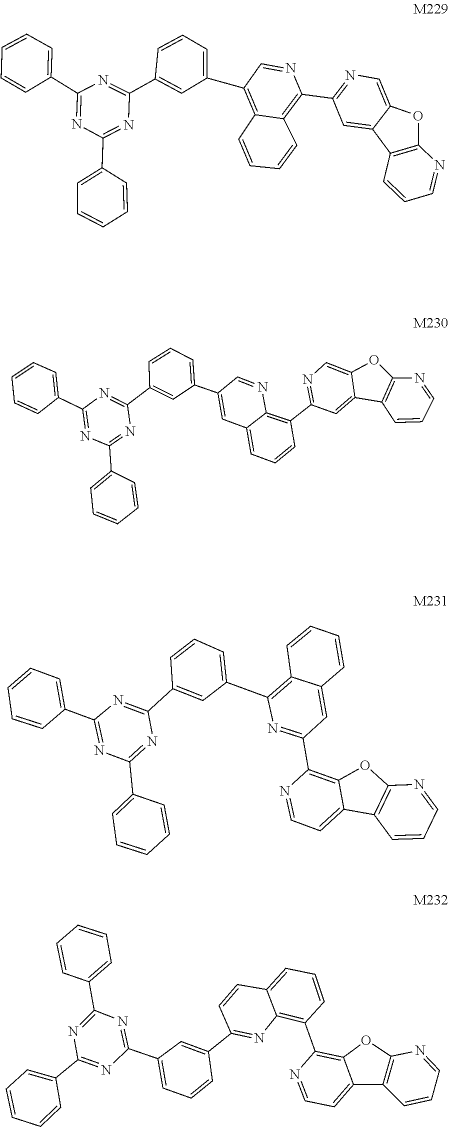 [SMILES: c1ccc(-c2nc(-c3ccccc3)nc(-c3cccc(-c4cnc(-c5cc6c(cn5)oc5ncccc56)c5ccccc45)c3)n2)cc1 ; c1ccc(-c2nc(-c3ccccc3)nc(-c3cccc(-c4cnc5c(-c6cc7c(cn6)oc6ncccc67)cccc5c4)c3)n2)cc1 ; c1ccc(-c2nc(-c3ccccc3)nc(-c3cccc(-c4nc(-c5nccc6c5oc5ncccc56)cc5ccccc45)c3)n2)cc1 ; c1ccc(-c2nc(-c3ccccc3)nc(-c3cccc(-c4ccc5cccc(-c6nccc7c6oc6ncccc67)c5n4)c3)n2)cc1]

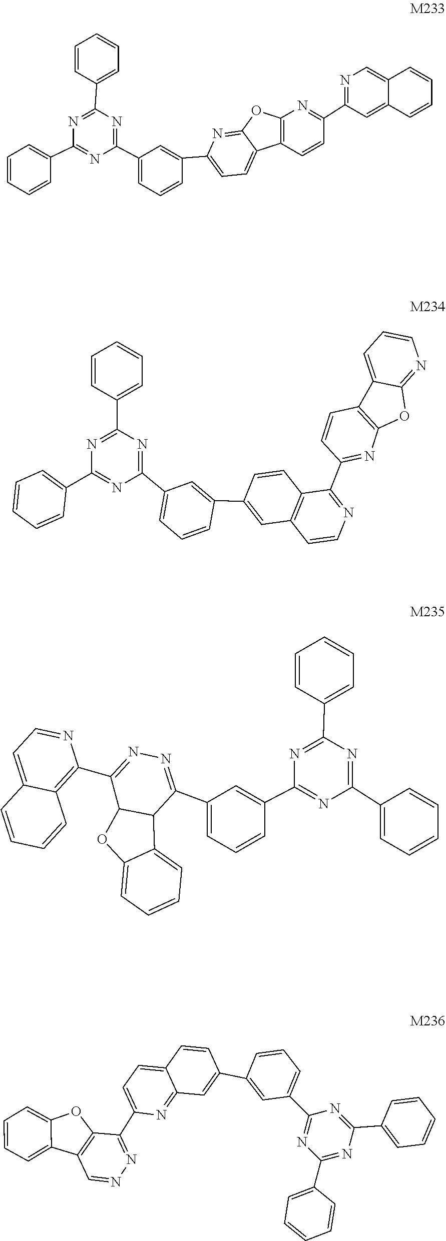 [SMILES: c1ccc(-c2nc(-c3ccccc3)nc(-c3cccc(-c4ccc5c(n4)oc4nc(-c6cc7ccccc7cn6)ccc45)c3)n2)cc1 ; c1ccc(-c2nc(-c3ccccc3)nc(-c3cccc(-c4ccc5c(-c6ccc7c(n6)oc6ncccc67)nccc5c4)c3)n2)cc1 ; c1ccc(-c2nc(-c3ccccc3)nc(-c3cccc(C4=NN=C(c5nccc6ccccc56)C5Oc6ccccc6C45)c3)n2)cc1 ; c1ccc(-c2nc(-c3ccccc3)nc(-c3cccc(-c4ccc5ccc(-c6nncc7c6oc6ccccc67)nc5c4)c3)n2)cc1]

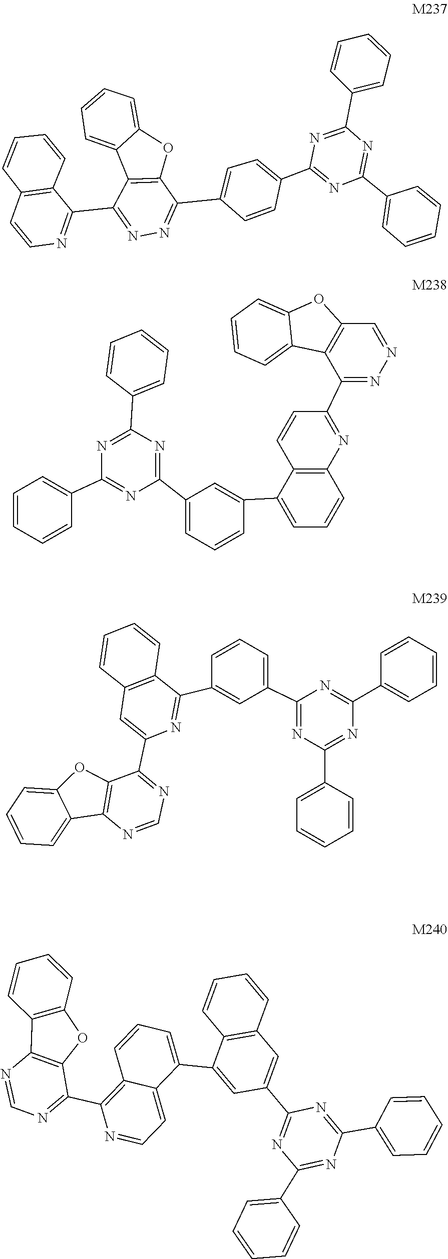 [SMILES: c1ccc(-c2nc(-c3ccccc3)nc(-c3ccc(-c4nnc(-c5nccc6ccccc56)c5c4oc4ccccc45)cc3)n2)cc1 ; c1ccc(-c2nc(-c3ccccc3)nc(-c3cccc(-c4cccc5nc(-c6nncc7oc8ccccc8c67)ccc45)c3)n2)cc1 ; c1ccc(-c2nc(-c3ccccc3)nc(-c3cccc(-c4nc(-c5ncnc6c5oc5ccccc56)cc5ccccc45)c3)n2)cc1 ; c1ccc(-c2nc(-c3ccccc3)nc(-c3cc(-c4cccc5c(-c6ncnc7c6oc6ccccc67)nccc45)c4ccccc4c3)n2)cc1]

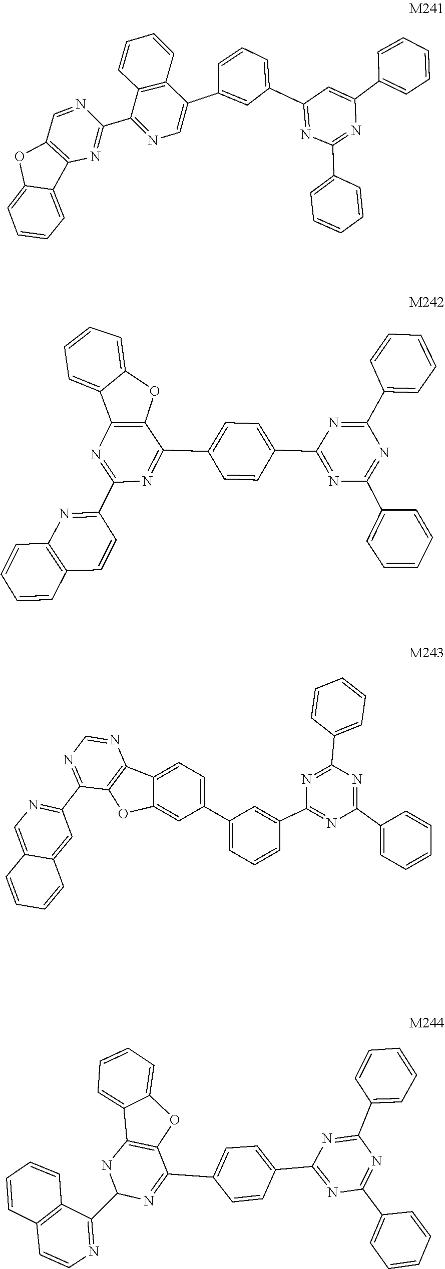

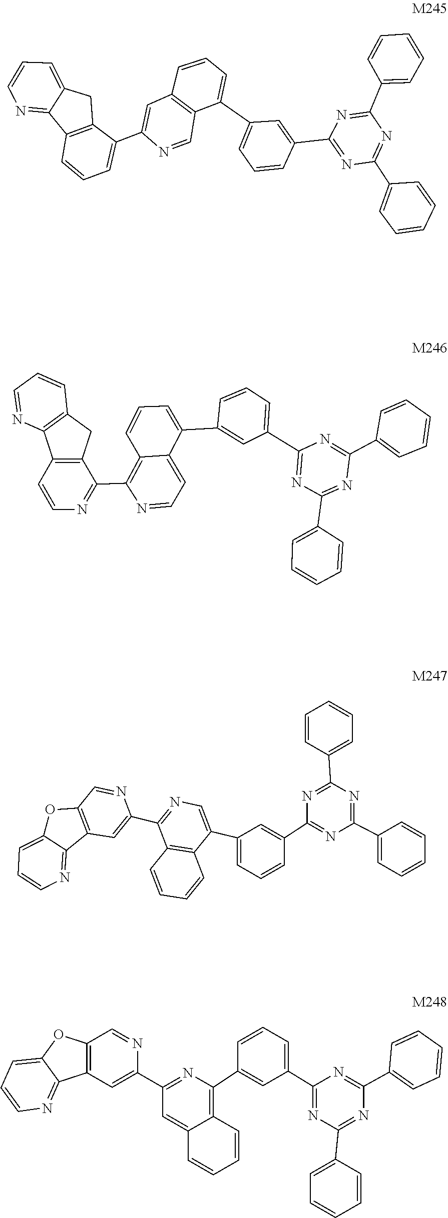 [SMILES: c1ccc(-c2nc(-c3ccccc3)nc(-c3cccc(-c4cccc5cc(-c6cccc7c6Cc6cccnc6-7)ncc45)c3)n2)cc1 ; c1ccc(-c2nc(-c3ccccc3)nc(-c3cccc(-c4cccc5c(-c6nccc7c6Cc6cccnc6-7)nccc45)c3)n2)cc1 ; c1ccc(-c2nc(-c3ccccc3)nc(-c3cccc(-c4cnc(-c5cc6c(cn5)oc5cccnc56)c5ccccc45)c3)n2)cc1 ; c1ccc(-c2nc(-c3ccccc3)nc(-c3cccc(-c4nc(-c5cc6c(cn5)oc5cccnc56)cc5ccccc45)c3)n2)cc1]

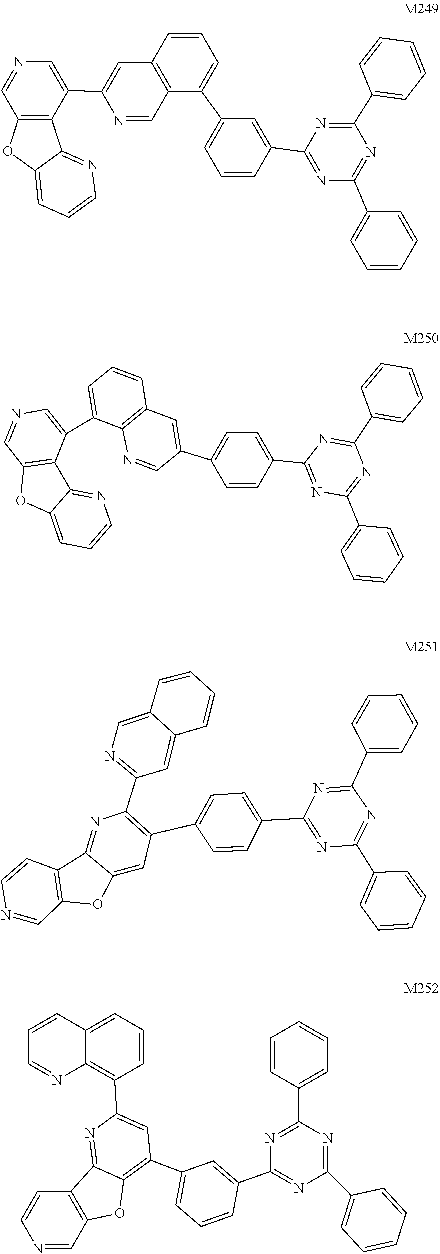 [SMILES: c1ccc(-c2nc(-c3ccccc3)nc(-c3cccc(-c4cccc5cc(-c6cncc7oc8cccnc8c67)ncc45)c3)n2)cc1 ; c1ccc(-c2nc(-c3ccccc3)nc(-c3ccc(-c4cnc5c(-c6cncc7oc8cccnc8c67)cccc5c4)cc3)n2)cc1 ; c1ccc(-c2nc(-c3ccccc3)nc(-c3ccc(-c4cc5oc6cnccc6c5nc4-c4cc5ccccc5cn4)cc3)n2)cc1 ; c1ccc(-c2nc(-c3ccccc3)nc(-c3cccc(-c4cc(-c5cccc6cccnc56)nc5c4oc4cnccc45)c3)n2)cc1]

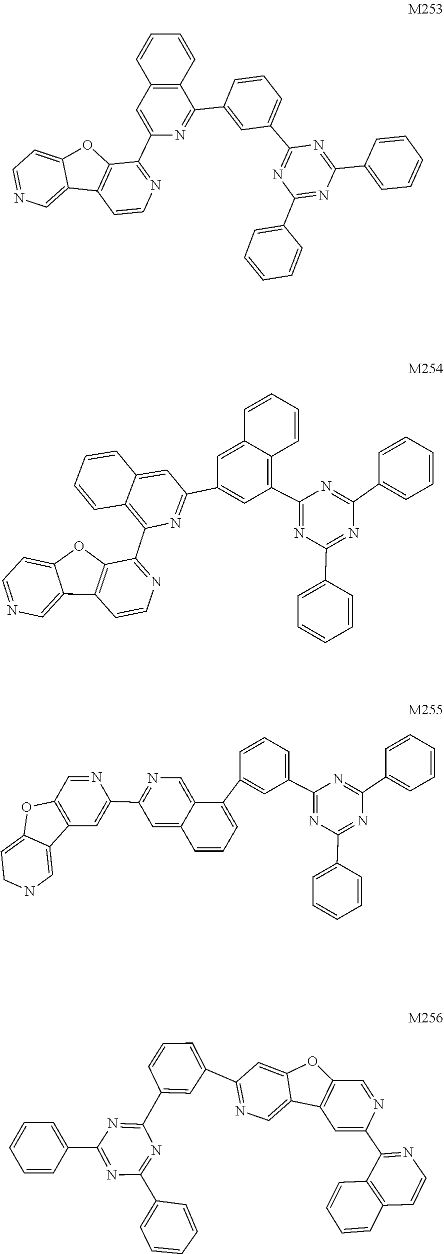 [SMILES: c1ccc(-c2nc(-c3ccccc3)nc(-c3cccc(-c4nc(-c5nccc6c5oc5ccncc56)cc5ccccc45)c3)n2)cc1 ; c1ccc(-c2nc(-c3ccccc3)nc(-c3cc(-c4cc5ccccc5c(-c5nccc6c5oc5ccncc56)n4)cc4ccccc34)n2)cc1 ; C1=c2oc3cnc(-c4cc5cccc(-c6cccc(-c7nc(-c8ccccc8)nc(-c8ccccc8)n7)c6)c5cn4)cc3c2=CNC1 ; c1ccc(-c2nc(-c3ccccc3)nc(-c3cccc(-c4cc5oc6cnc(-c7nccc8ccccc78)cc6c5cn4)c3)n2)cc1]

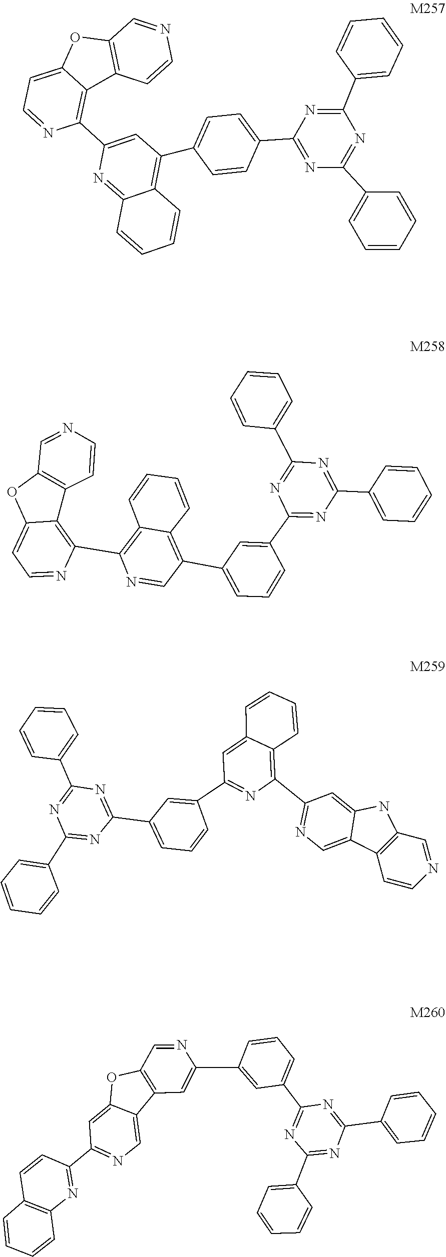 [SMILES: c1ccc(-c2nc(-c3ccccc3)nc(-c3ccc(-c4cc(-c5nccc6oc7cnccc7c56)nc5ccccc45)cc3)n2)cc1 ; c1ccc(-c2nc(-c3ccccc3)nc(-c3cccc(-c4cnc(-c5nccc6oc7cnccc7c56)c5ccccc45)c3)n2)cc1 ; c1ccc(-c2nc(-c3ccccc3)nc(-c3cccc(-c4cc5ccccc5c(-c5cc6[nH]c7cnccc7c6cn5)n4)c3)n2)cc1 ; c1ccc(-c2nc(-c3ccccc3)nc(-c3cccc(-c4cc5c(cn4)oc4cc(-c6ccc7ccccc7n6)ncc45)c3)n2)cc1]

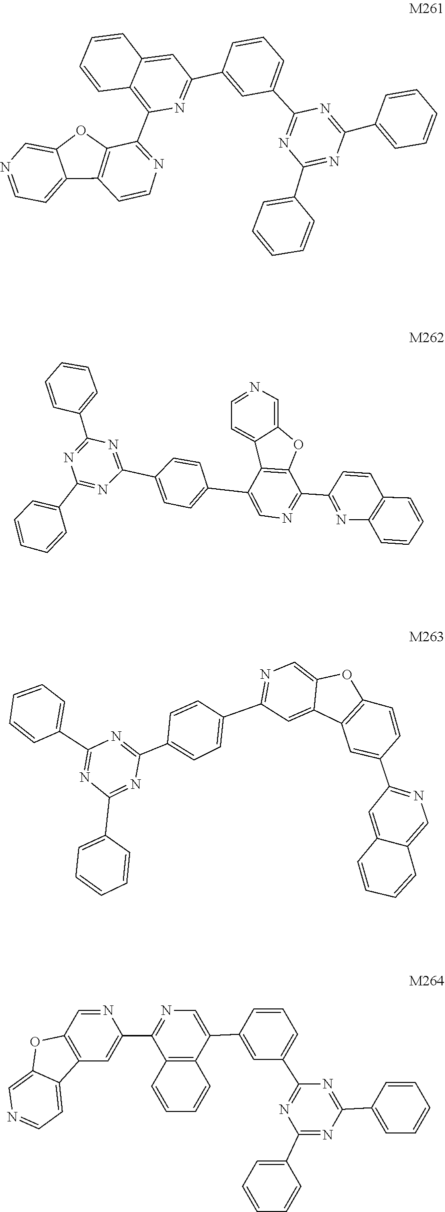 [SMILES: c1ccc(-c2nc(-c3ccccc3)nc(-c3cccc(-c4cc5ccccc5c(-c5nccc6c5oc5cnccc56)n4)c3)n2)cc1 ; c1ccc(-c2nc(-c3ccccc3)nc(-c3ccc(-c4cnc(-c5ccc6ccccc6n5)c5oc6cnccc6c45)cc3)n2)cc1 ; c1ccc(-c2nc(-c3ccccc3)nc(-c3ccc(-c4cc5c(cn4)oc4ccc(-c6cc7ccccc7cn6)cc45)cc3)n2)cc1 ; c1ccc(-c2nc(-c3ccccc3)nc(-c3cccc(-c4cnc(-c5cc6c(cn5)oc5cnccc56)c5ccccc45)c3)n2)cc1]

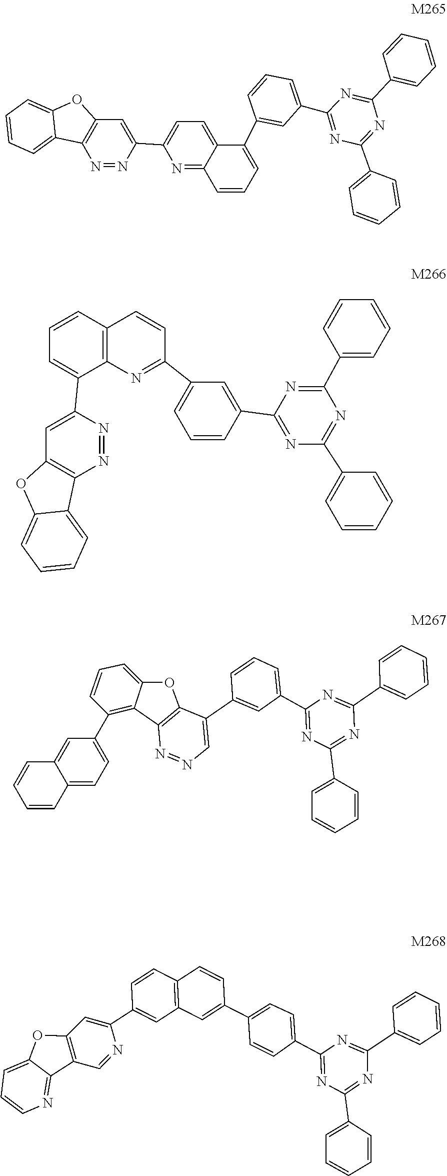 [SMILES: c1ccc(-c2nc(-c3ccccc3)nc(-c3cccc(-c4cccc5nc(-c6cc7oc8ccccc8c7nn6)ccc45)c3)n2)cc1 ; c1ccc(-c2nc(-c3ccccc3)nc(-c3cccc(-c4ccc5cccc(-c6cc7oc8ccccc8c7nn6)c5n4)c3)n2)cc1 ; c1ccc(-c2nc(-c3ccccc3)nc(-c3cccc(-c4cnnc5c4oc4cccc(-c6ccc7ccccc7c6)c45)c3)n2)cc1 ; c1ccc(-c2nc(-c3ccccc3)nc(-c3ccc(-c4ccc5ccc(-c6cc7oc8cccnc8c7cn6)cc5c4)cc3)n2)cc1]

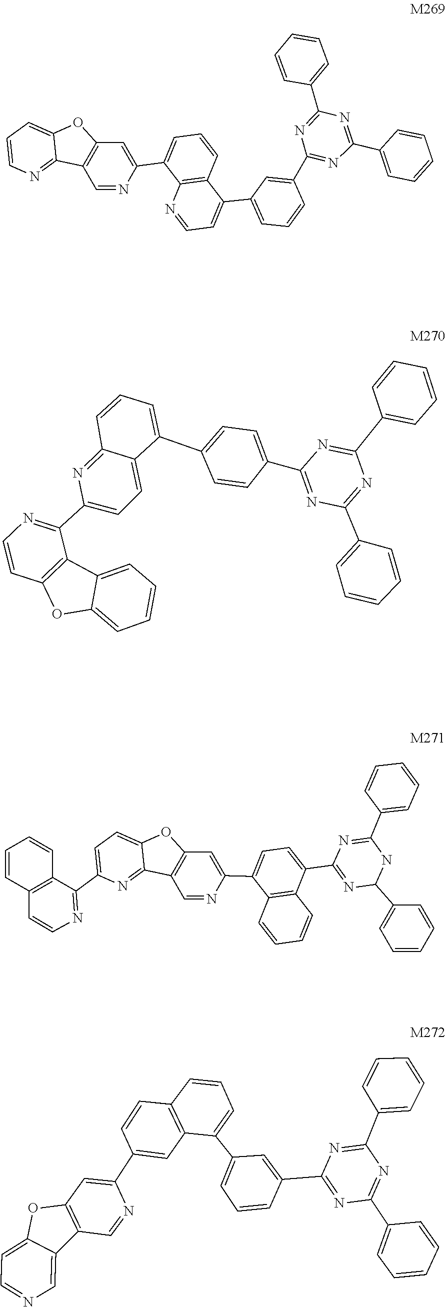

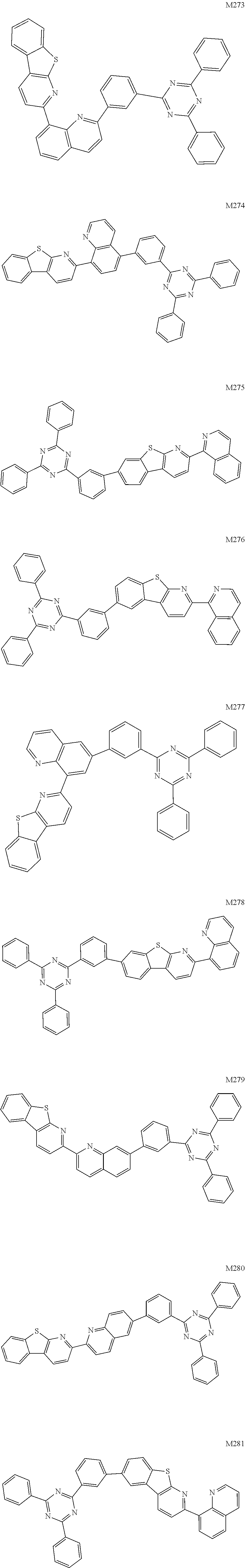

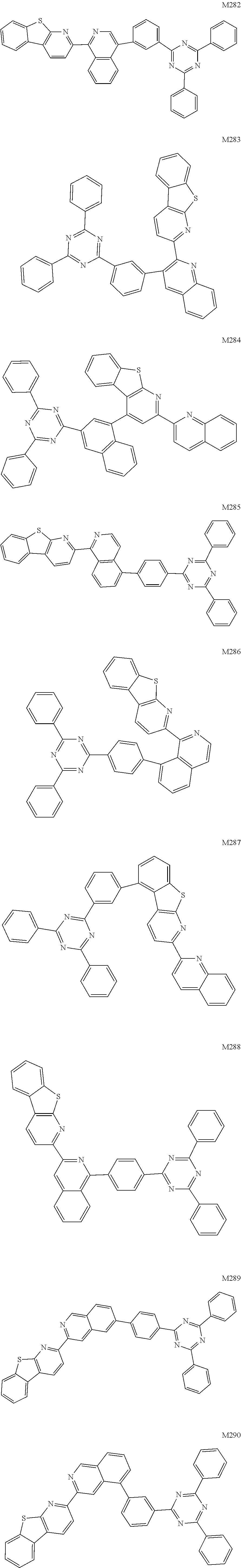

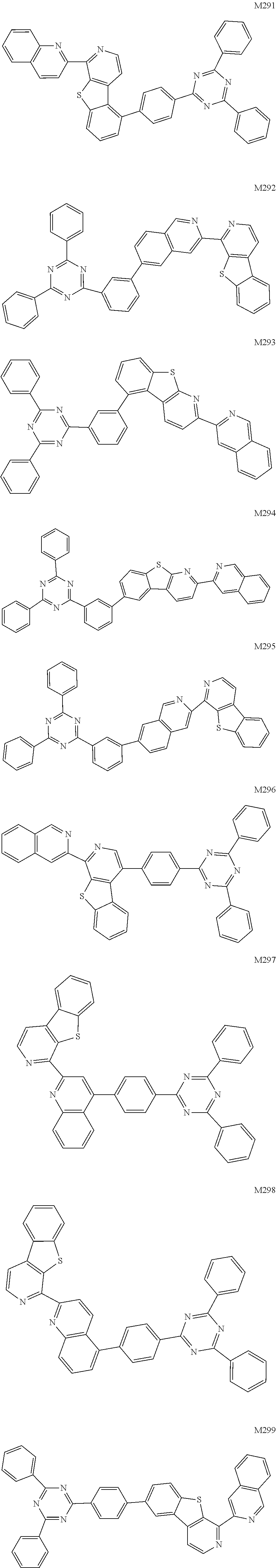

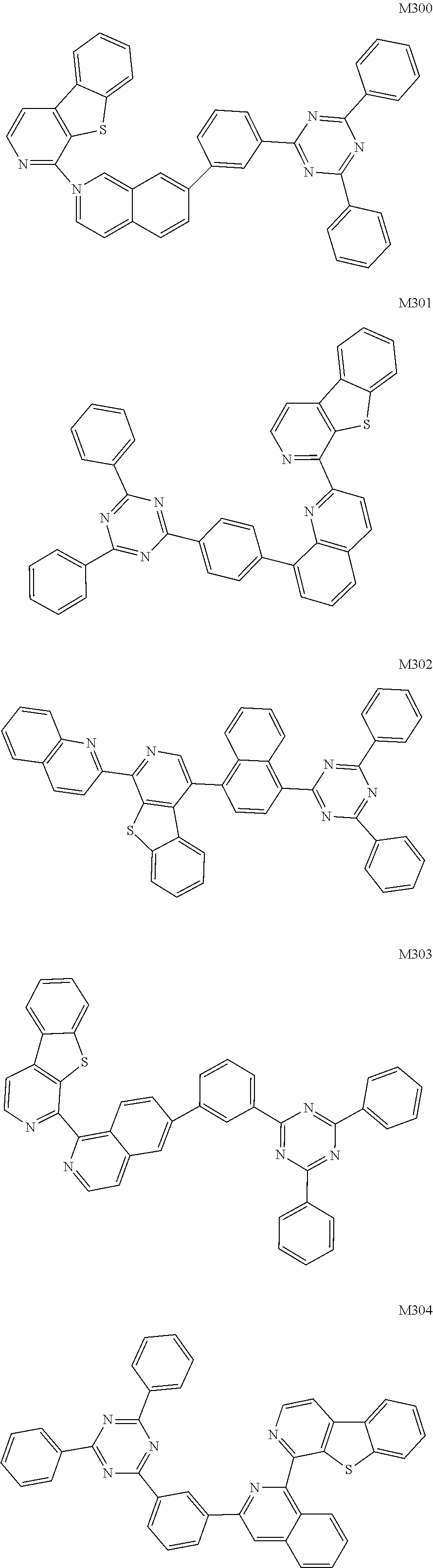

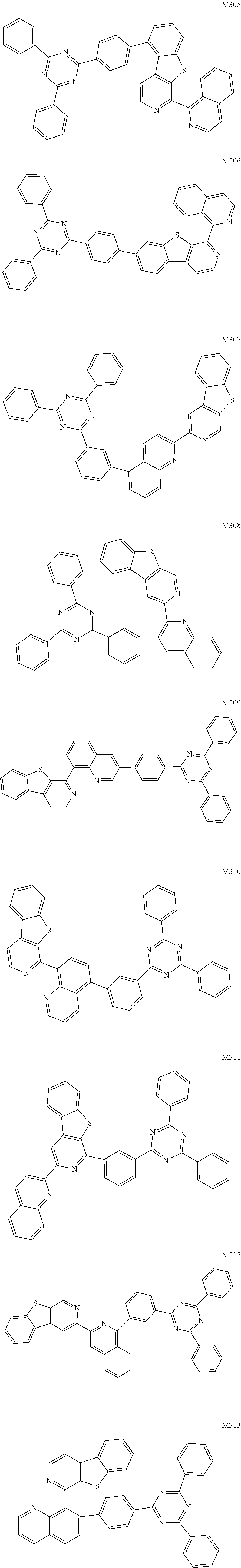

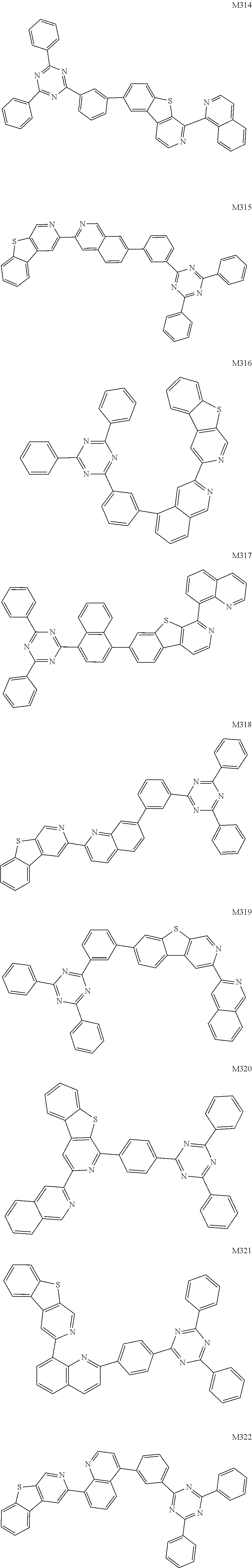

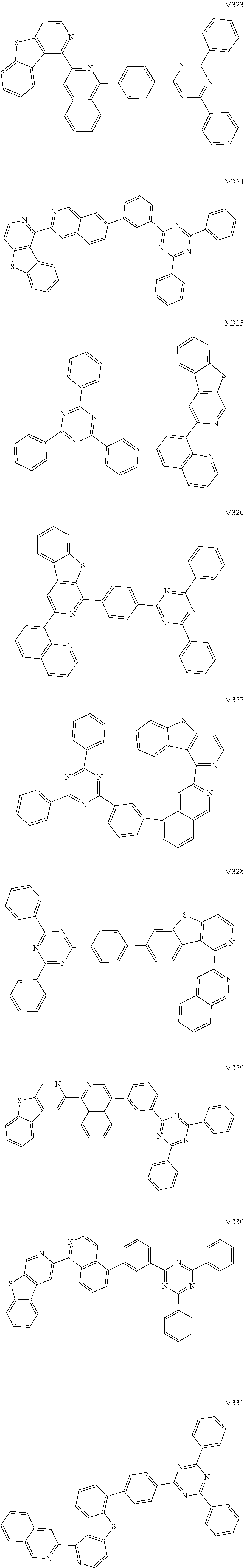

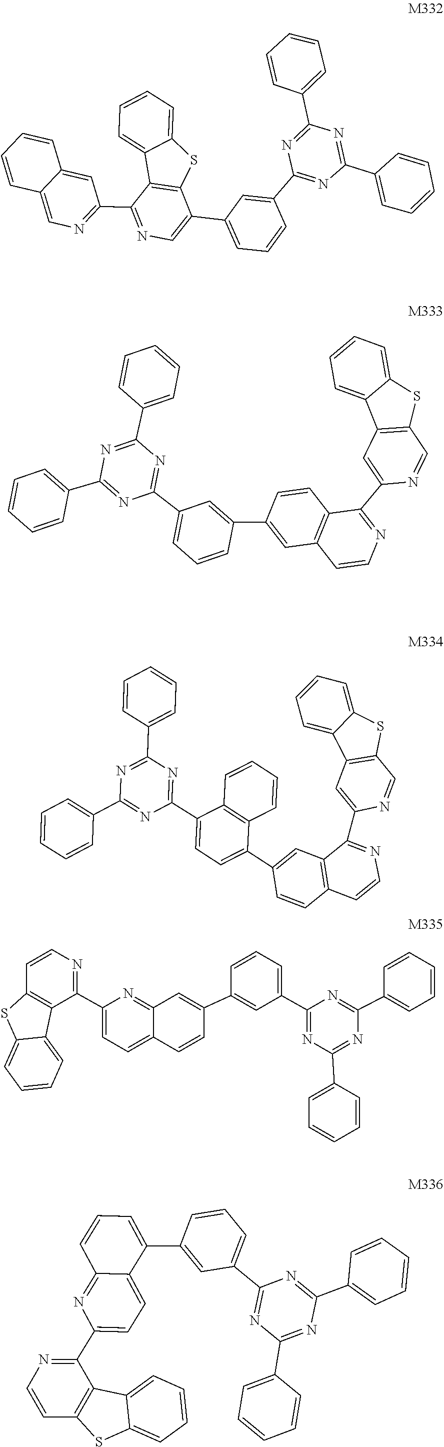 [SMILES: c1ccc(-c2nc(-c3ccccc3)nc(-c3cccc(-c4cnc(-c5cc6ccccc6cn5)c5c4sc4ccccc45)c3)n2)cc1 ; c1ccc(-c2nc(-c3ccccc3)nc(-c3cccc(-c4ccc5c(-c6cc7c(cn6)sc6ccccc67)nccc5c4)c3)n2)cc1 ; c1ccc(-c2nc(-c3ccccc3)nc(-c3ccc(-c4ccc5ccnc(-c6cc7c(cn6)sc6ccccc67)c5c4)c4ccccc34)n2)cc1 ; c1ccc(-c2nc(-c3ccccc3)nc(-c3cccc(-c4ccc5ccc(-c6nccc7sc8ccccc8c67)nc5c4)c3)n2)cc1 ; c1ccc(-c2nc(-c3ccccc3)nc(-c3cccc(-c4cccc5nc(-c6nccc7sc8ccccc8c67)ccc45)c3)n2)cc1]

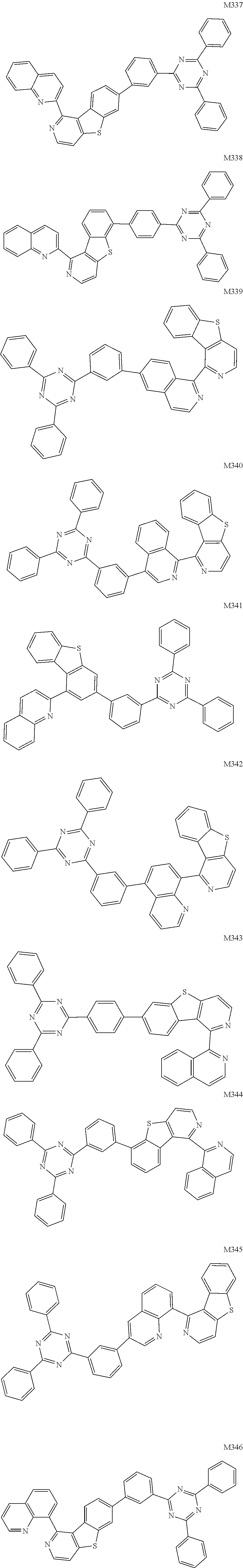

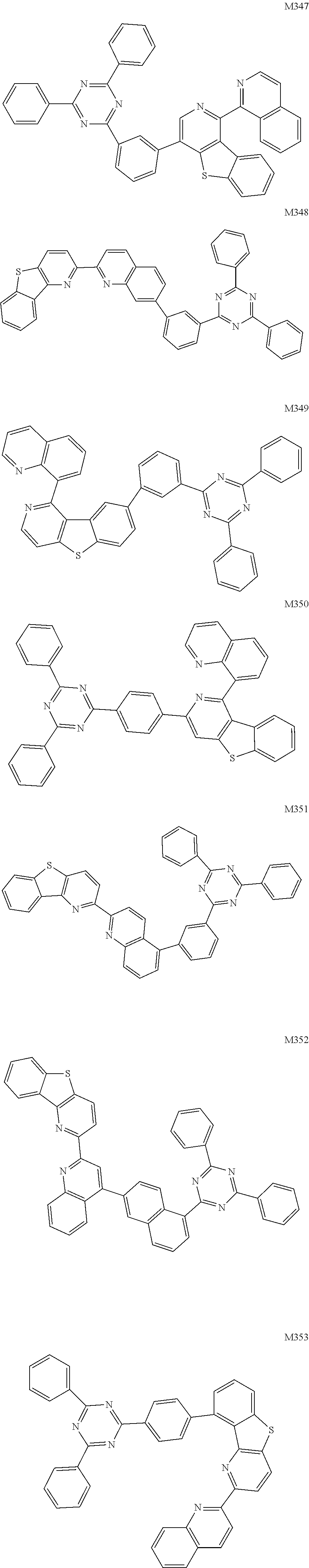

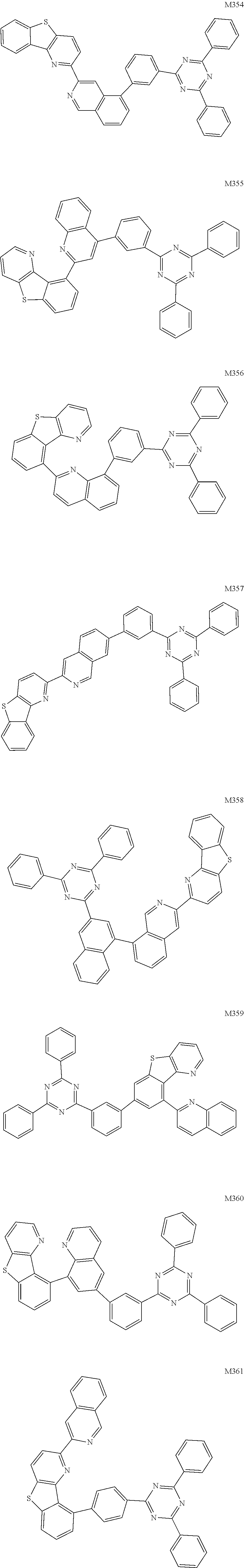

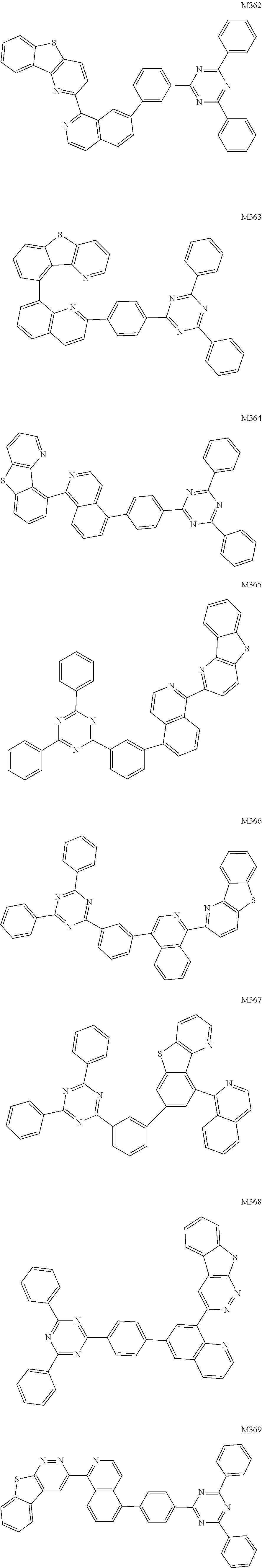

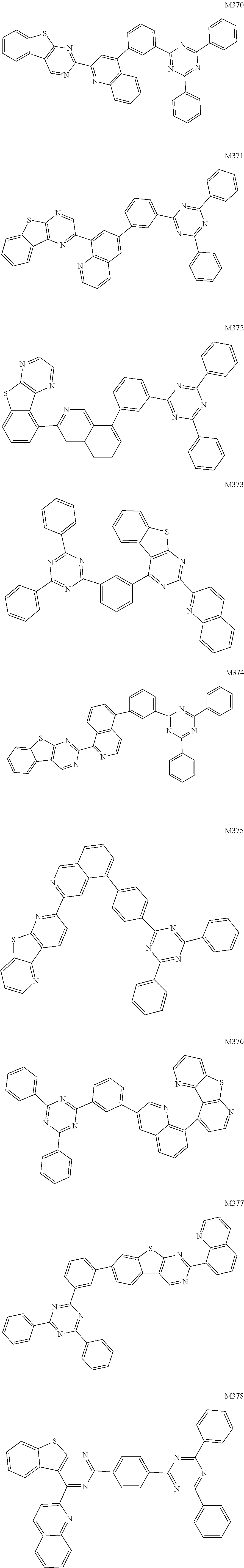

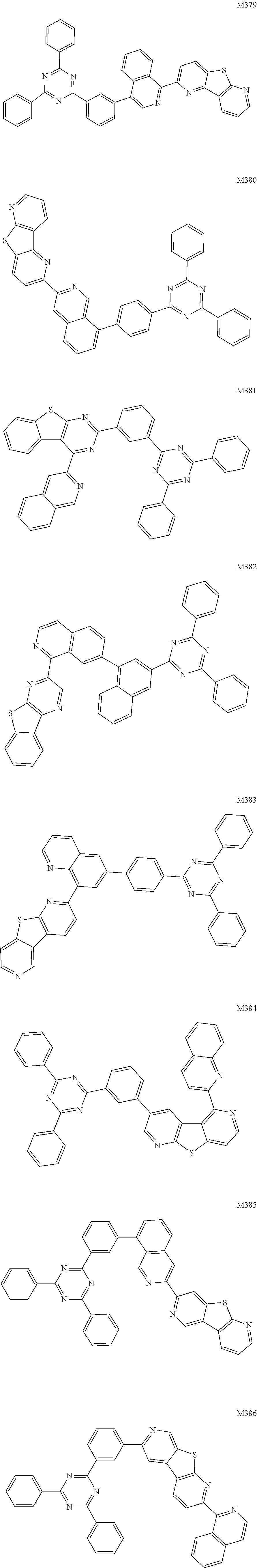

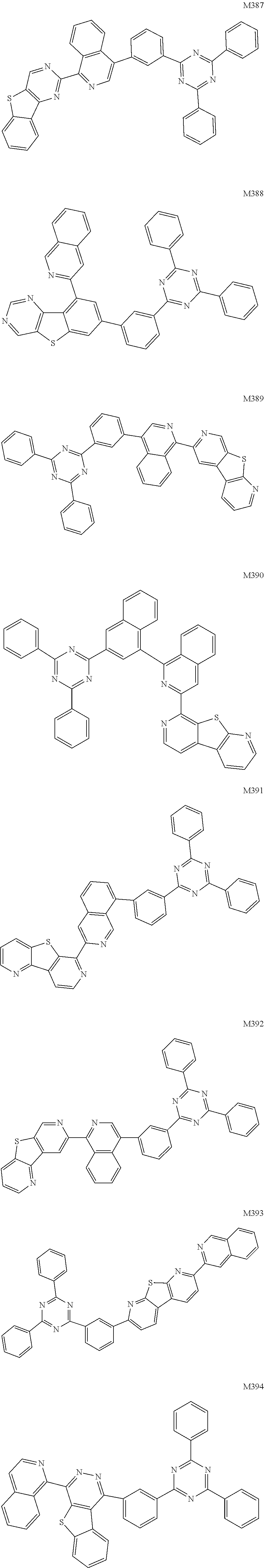

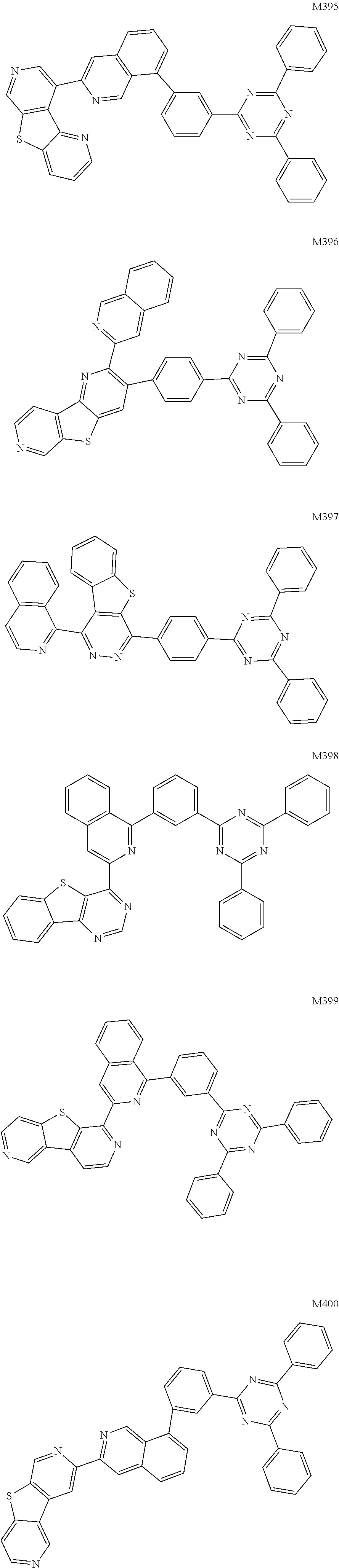 [SMILES: c1ccc(-c2nc(-c3ccccc3)nc(-c3cccc(-c4cccc5cc(-c6cncc7sc8cccnc8c67)ncc45)c3)n2)cc1 ; c1ccc(-c2nc(-c3ccccc3)nc(-c3ccc(-c4cc5sc6cnccc6c5nc4-c4cc5ccccc5cn4)cc3)n2)cc1 ; c1ccc(-c2nc(-c3ccccc3)nc(-c3ccc(-c4nnc(-c5nccc6ccccc56)c5c4sc4ccccc45)cc3)n2)cc1 ; c1ccc(-c2nc(-c3ccccc3)nc(-c3cccc(-c4nc(-c5ncnc6c5sc5ccccc56)cc5ccccc45)c3)n2)cc1 ; c1ccc(-c2nc(-c3ccccc3)nc(-c3cccc(-c4nc(-c5nccc6c5sc5ccncc56)cc5ccccc45)c3)n2)cc1 ; c1ccc(-c2nc(-c3ccccc3)nc(-c3cccc(-c4cccc5cc(-c6cc7c(cn6)sc6ccncc67)ncc45)c3)n2)cc1]

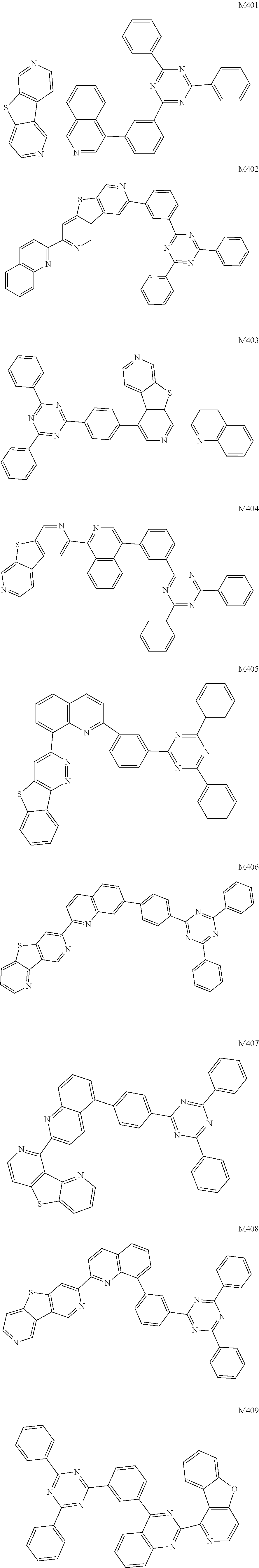

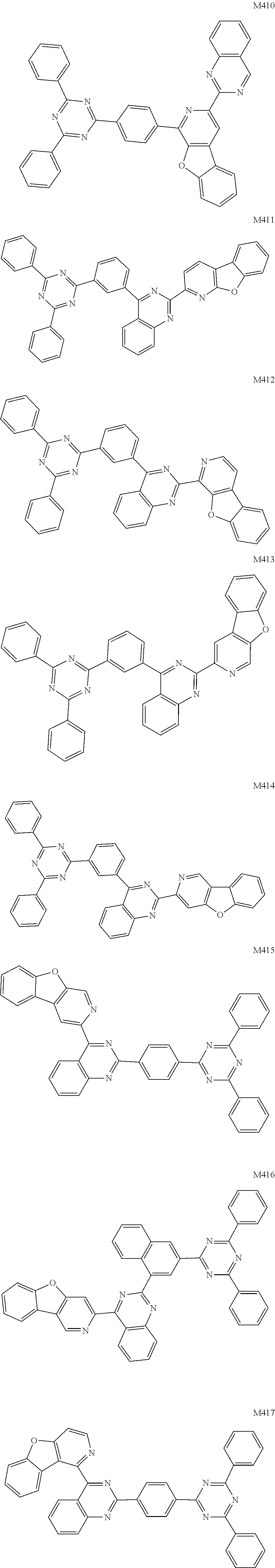

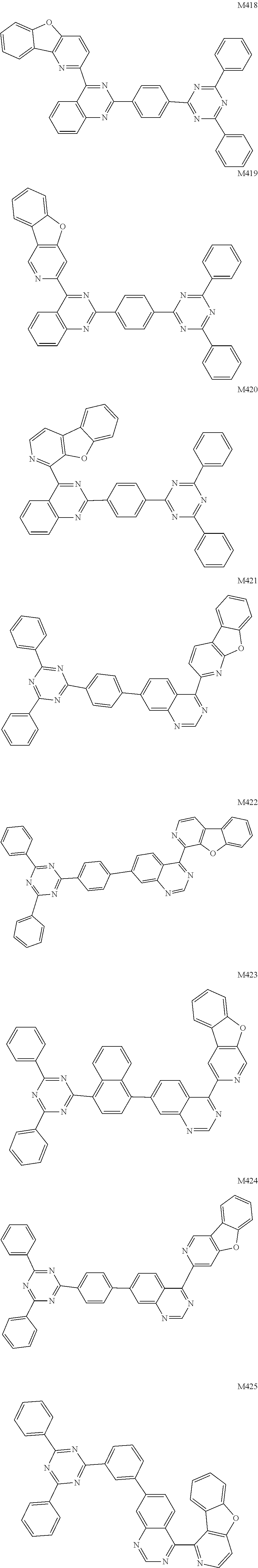

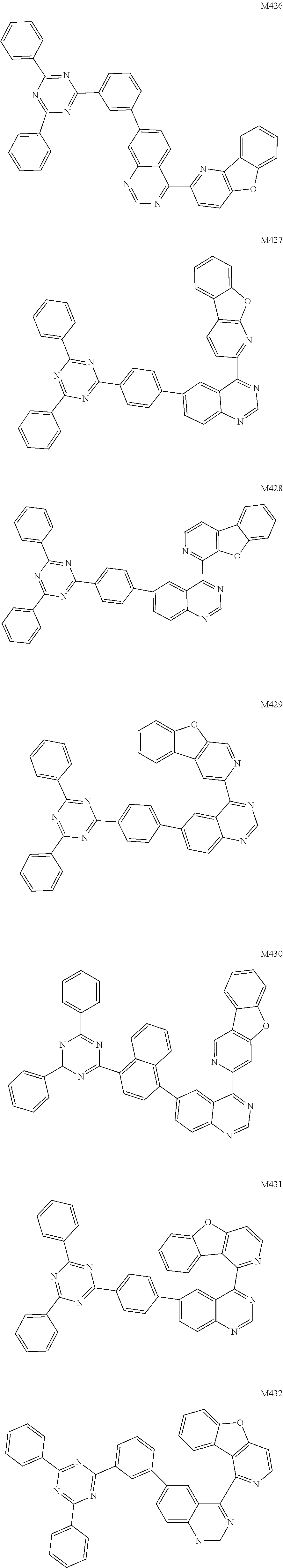

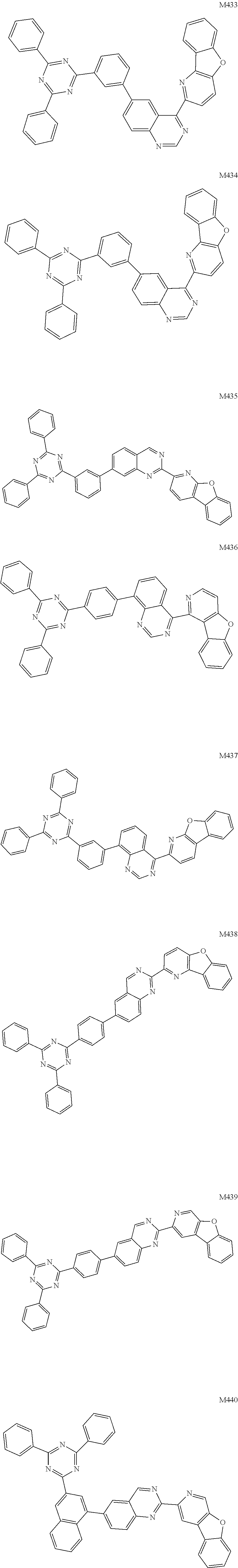

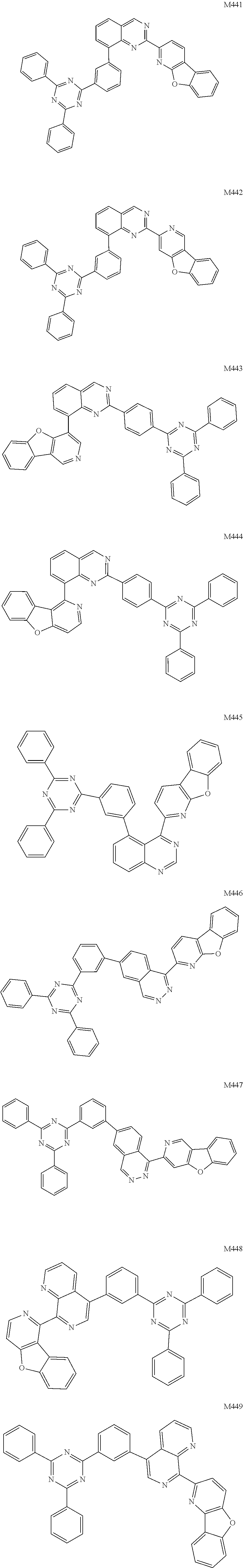

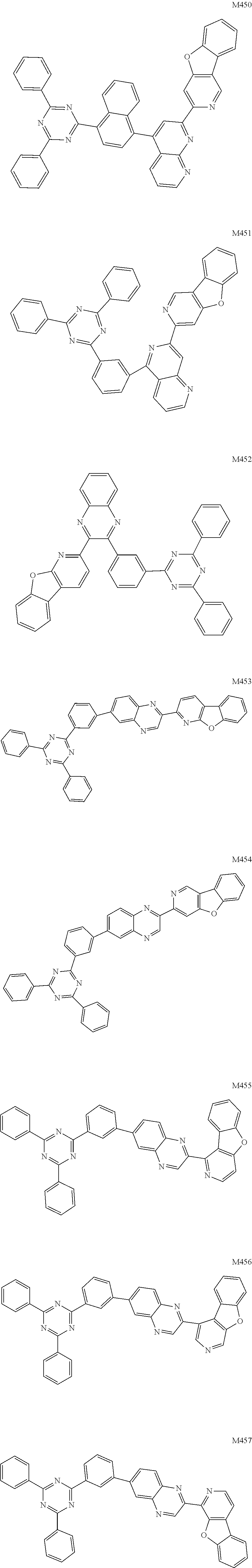

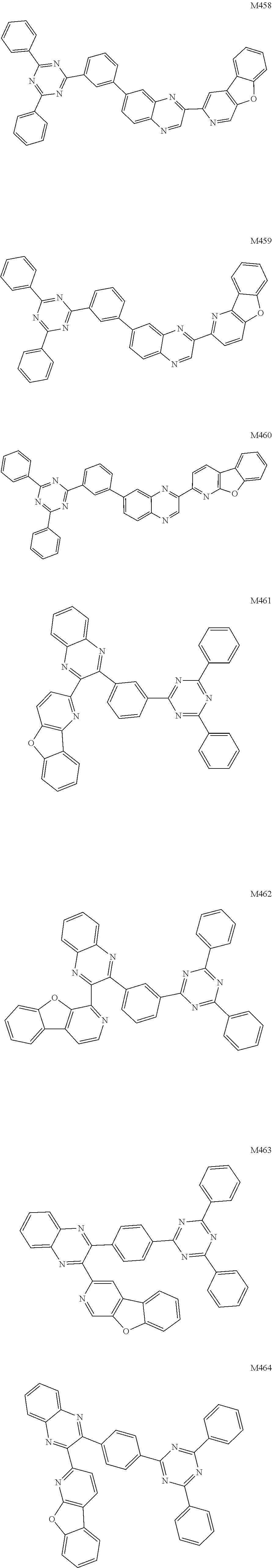

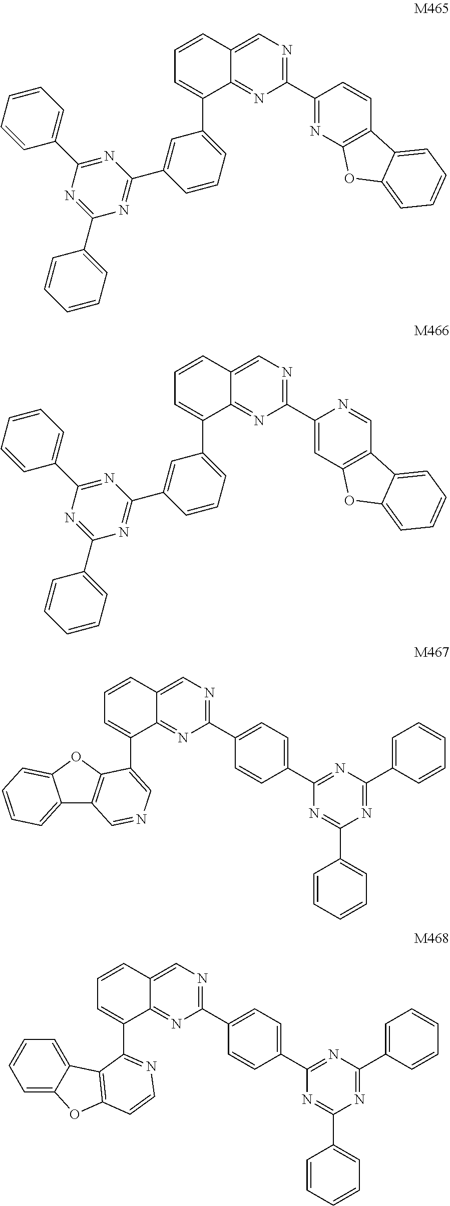 [SMILES: c1ccc(-c2nc(-c3ccccc3)nc(-c3cccc(-c4cccc5cnc(-c6ccc7c(n6)oc6ccccc67)nc45)c3)n2)cc1 ; c1ccc(-c2nc(-c3ccccc3)nc(-c3cccc(-c4cccc5cnc(-c6cc7oc8ccccc8c7cn6)nc45)c3)n2)cc1 ; c1ccc(-c2nc(-c3ccccc3)nc(-c3ccc(-c4ncc5cccc(-c6cncc7c6oc6ccccc67)c5n4)cc3)n2)cc1 ; c1ccc(-c2nc(-c3ccccc3)nc(-c3ccc(-c4ncc5cccc(-c6nccc7oc8ccccc8c67)c5n4)cc3)n2)cc1]

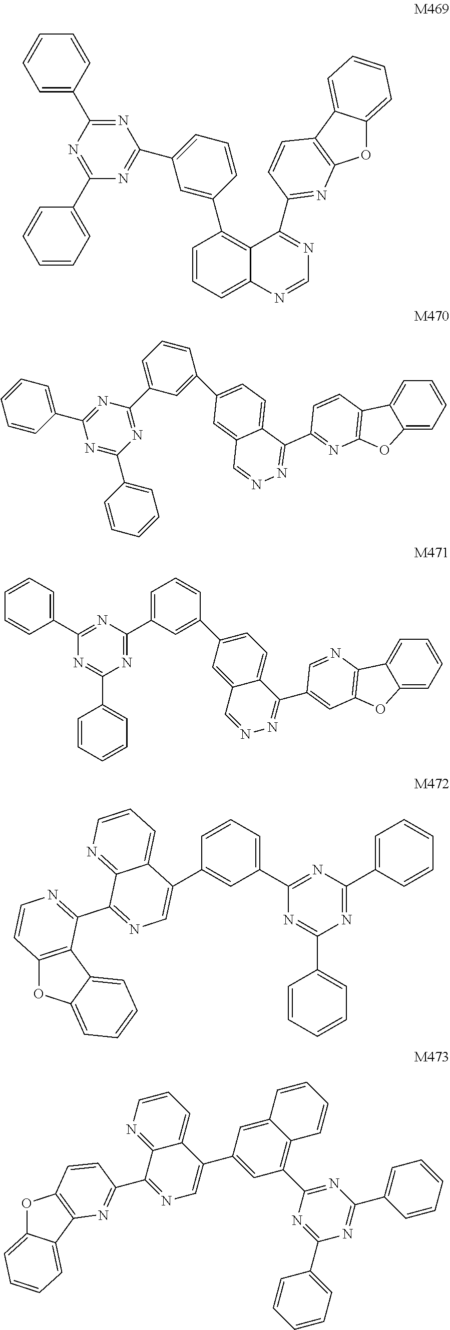 [SMILES: c1ccc(-c2nc(-c3ccccc3)nc(-c3cccc(-c4cccc5ncnc(-c6ccc7c(n6)oc6ccccc67)c45)c3)n2)cc1 ; c1ccc(-c2nc(-c3ccccc3)nc(-c3cccc(-c4ccc5c(-c6ccc7c(n6)oc6ccccc67)nncc5c4)c3)n2)cc1 ; c1ccc(-c2nc(-c3ccccc3)nc(-c3cccc(-c4ccc5c(-c6cnc7c(c6)oc6ccccc67)nncc5c4)c3)n2)cc1 ; c1ccc(-c2nc(-c3ccccc3)nc(-c3cccc(-c4cnc(-c5nccc6oc7ccccc7c56)c5ncccc45)c3)n2)cc1 ; c1ccc(-c2nc(-c3ccccc3)nc(-c3cc(-c4cnc(-c5ccc6oc7ccccc7c6n5)c5ncccc45)cc4ccccc34)n2)cc1]

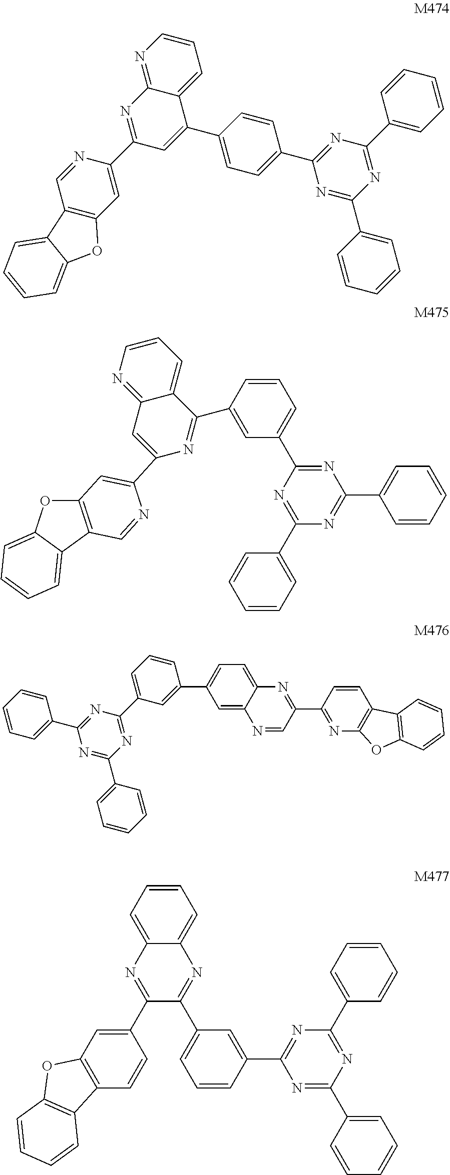 [SMILES: c1ccc(-c2nc(-c3ccccc3)nc(-c3ccc(-c4cc(-c5cc6oc7ccccc7c6cn5)nc5ncccc45)cc3)n2)cc1 ; c1ccc(-c2nc(-c3ccccc3)nc(-c3cccc(-c4nc(-c5cc6oc7ccccc7c6cn5)cc5ncccc45)c3)n2)cc1 ; c1ccc(-c2nc(-c3ccccc3)nc(-c3cccc(-c4ccc5nc(-c6ccc7c(n6)oc6ccccc67)cnc5c4)c3)n2)cc1 ; c1ccc(-c2nc(-c3ccccc3)nc(-c3cccc(-c4nc5ccccc5nc4-c4ccc5c(c4)oc4ccccc45)c3)n2)cc1]

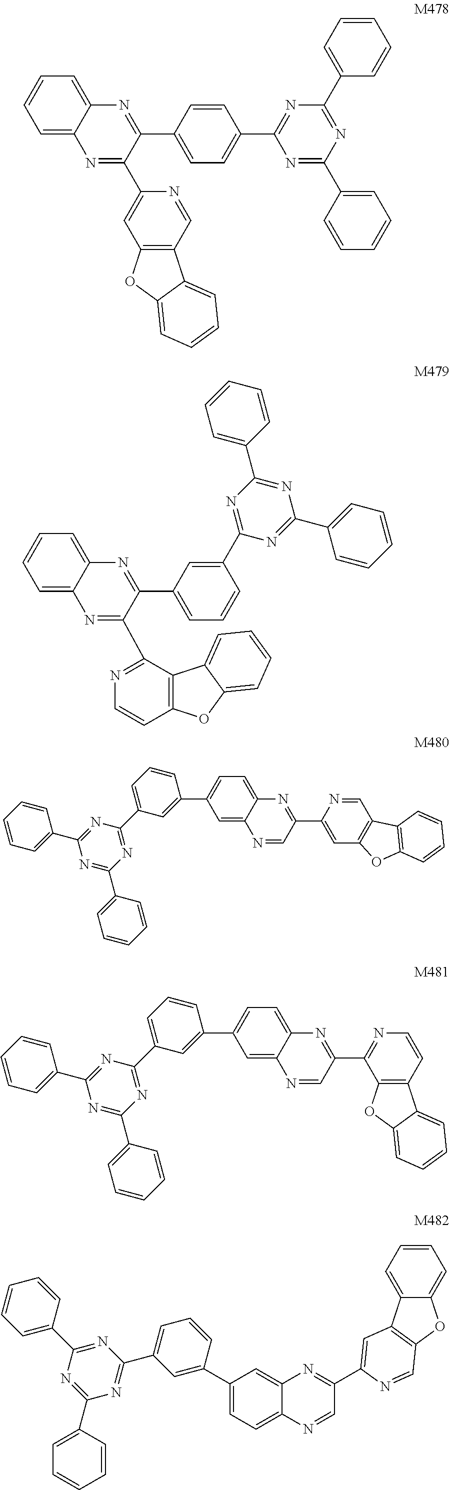 [SMILES: c1ccc(-c2nc(-c3ccccc3)nc(-c3ccc(-c4nc5ccccc5nc4-c4cc5oc6ccccc6c5cn4)cc3)n2)cc1 ; c1ccc(-c2nc(-c3ccccc3)nc(-c3cccc(-c4nc5ccccc5nc4-c4nccc5oc6ccccc6c45)c3)n2)cc1 ; c1ccc(-c2nc(-c3ccccc3)nc(-c3cccc(-c4ccc5nc(-c6cc7oc8ccccc8c7cn6)cnc5c4)c3)n2)cc1 ; c1ccc(-c2nc(-c3ccccc3)nc(-c3cccc(-c4ccc5nc(-c6nccc7c6oc6ccccc67)cnc5c4)c3)n2)cc1 ; c1ccc(-c2nc(-c3ccccc3)nc(-c3cccc(-c4ccc5ncc(-c6cc7c(cn6)oc6ccccc67)nc5c4)c3)n2)cc1]

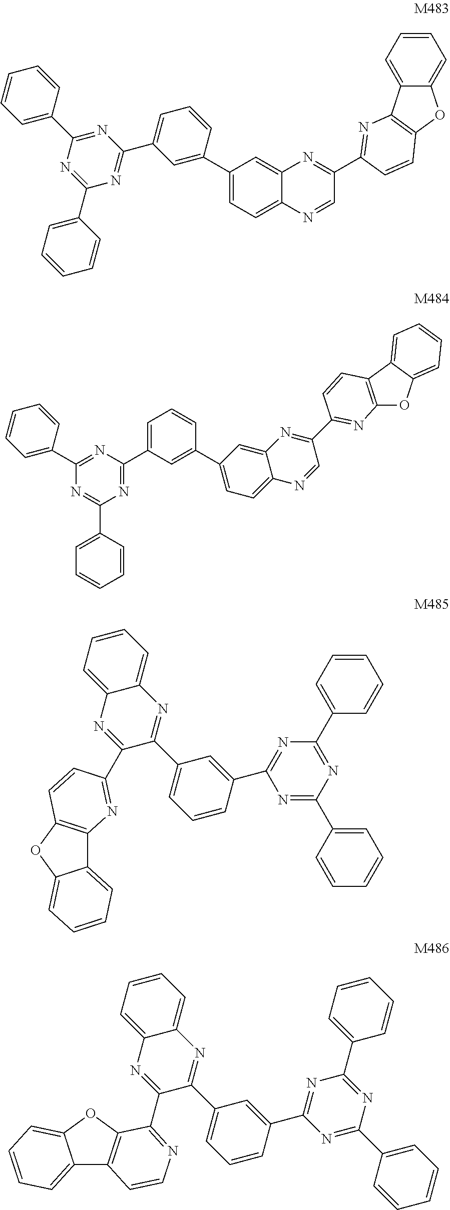 [SMILES: c1ccc(-c2nc(-c3ccccc3)nc(-c3cccc(-c4ccc5ncc(-c6ccc7oc8ccccc8c7n6)nc5c4)c3)n2)cc1 ; c1ccc(-c2nc(-c3ccccc3)nc(-c3cccc(-c4ccc5ncc(-c6ccc7c(n6)oc6ccccc67)nc5c4)c3)n2)cc1 ; c1ccc(-c2nc(-c3ccccc3)nc(-c3cccc(-c4nc5ccccc5nc4-c4ccc5oc6ccccc6c5n4)c3)n2)cc1 ; c1ccc(-c2nc(-c3ccccc3)nc(-c3cccc(-c4nc5ccccc5nc4-c4nccc5c4oc4ccccc45)c3)n2)cc1]

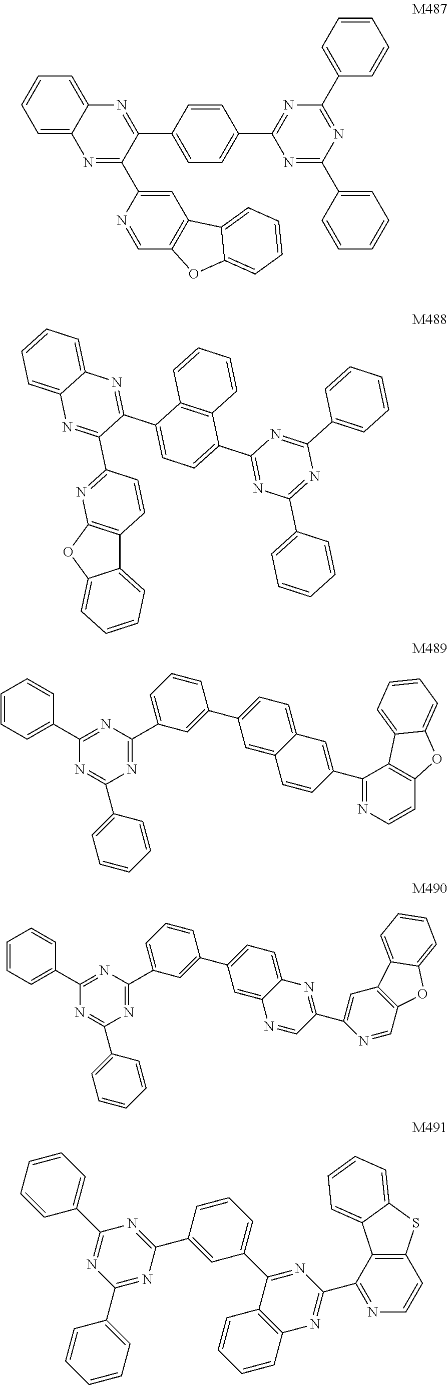 [SMILES: c1ccc(-c2nc(-c3ccccc3)nc(-c3ccc(-c4nc5ccccc5nc4-c4cc5c(cn4)oc4ccccc45)cc3)n2)cc1 ; c1ccc(-c2nc(-c3ccccc3)nc(-c3ccc(-c4nc5ccccc5nc4-c4ccc5c(n4)oc4ccccc45)c4ccccc34)n2)cc1 ; c1ccc(-c2nc(-c3ccccc3)nc(-c3cccc(-c4ccc5cc(-c6nccc7oc8ccccc8c67)ccc5c4)c3)n2)cc1 ; c1ccc(-c2nc(-c3ccccc3)nc(-c3cccc(-c4ccc5nc(-c6cc7c(cn6)oc6ccccc67)cnc5c4)c3)n2)cc1 ; c1ccc(-c2nc(-c3ccccc3)nc(-c3cccc(-c4nc(-c5nccc6sc7ccccc7c56)nc5ccccc45)c3)n2)cc1]

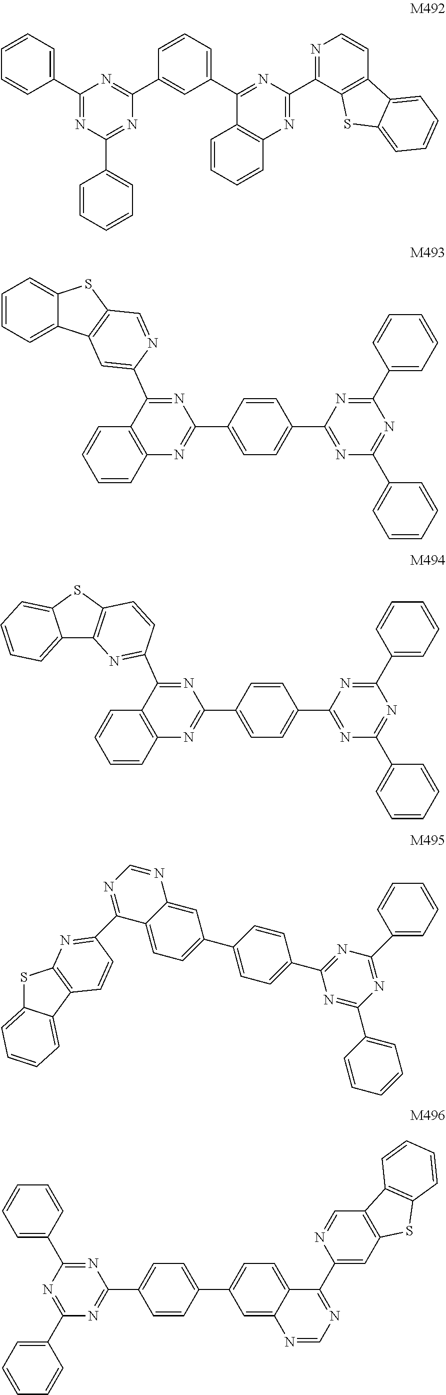 [SMILES: c1ccc(-c2nc(-c3ccccc3)nc(-c3cccc(-c4nc(-c5nccc6c5sc5ccccc56)nc5ccccc45)c3)n2)cc1 ; c1ccc(-c2nc(-c3ccccc3)nc(-c3ccc(-c4nc(-c5cc6c(cn5)sc5ccccc56)c5ccccc5n4)cc3)n2)cc1 ; c1ccc(-c2nc(-c3ccccc3)nc(-c3ccc(-c4nc(-c5ccc6sc7ccccc7c6n5)c5ccccc5n4)cc3)n2)cc1 ; c1ccc(-c2nc(-c3ccccc3)nc(-c3ccc(-c4ccc5c(-c6ccc7c(n6)sc6ccccc67)ncnc5c4)cc3)n2)cc1 ; c1ccc(-c2nc(-c3ccccc3)nc(-c3ccc(-c4ccc5c(-c6cc7sc8ccccc8c7cn6)ncnc5c4)cc3)n2)cc1]

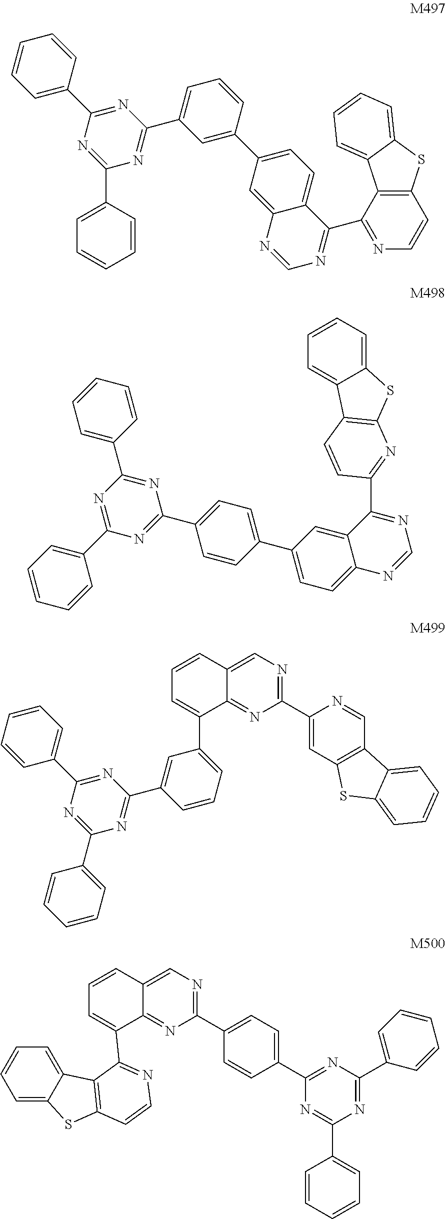 [SMILES: c1ccc(-c2nc(-c3ccccc3)nc(-c3cccc(-c4ccc5c(-c6nccc7sc8ccccc8c67)ncnc5c4)c3)n2)cc1 ; c1ccc(-c2nc(-c3ccccc3)nc(-c3ccc(-c4ccc5ncnc(-c6ccc7c(n6)sc6ccccc67)c5c4)cc3)n2)cc1 ; c1ccc(-c2nc(-c3ccccc3)nc(-c3cccc(-c4cccc5cnc(-c6cc7sc8ccccc8c7cn6)nc45)c3)n2)cc1 ; c1ccc(-c2nc(-c3ccccc3)nc(-c3ccc(-c4ncc5cccc(-c6nccc7sc8ccccc8c67)c5n4)cc3)n2)cc1]

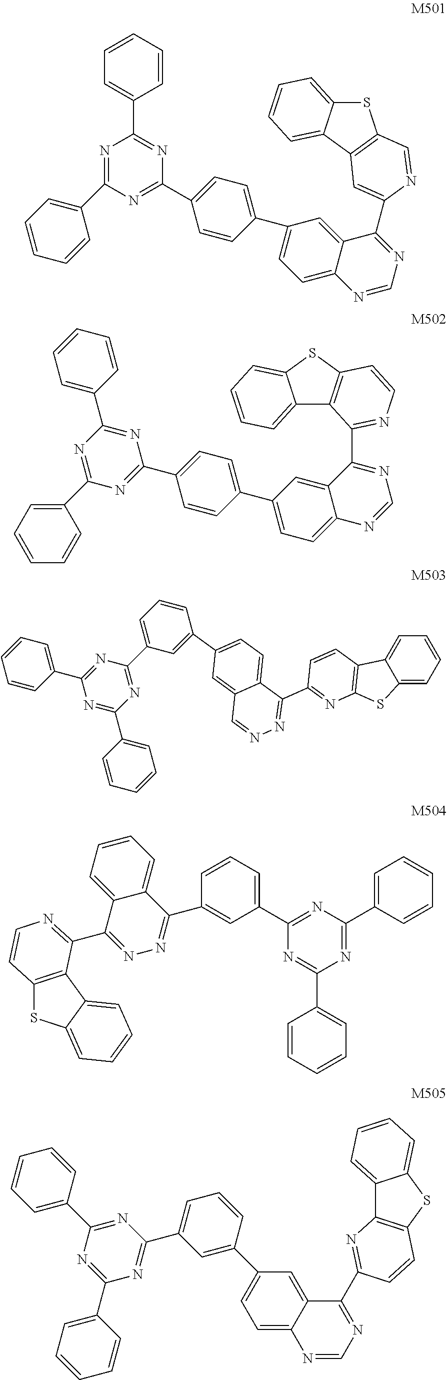

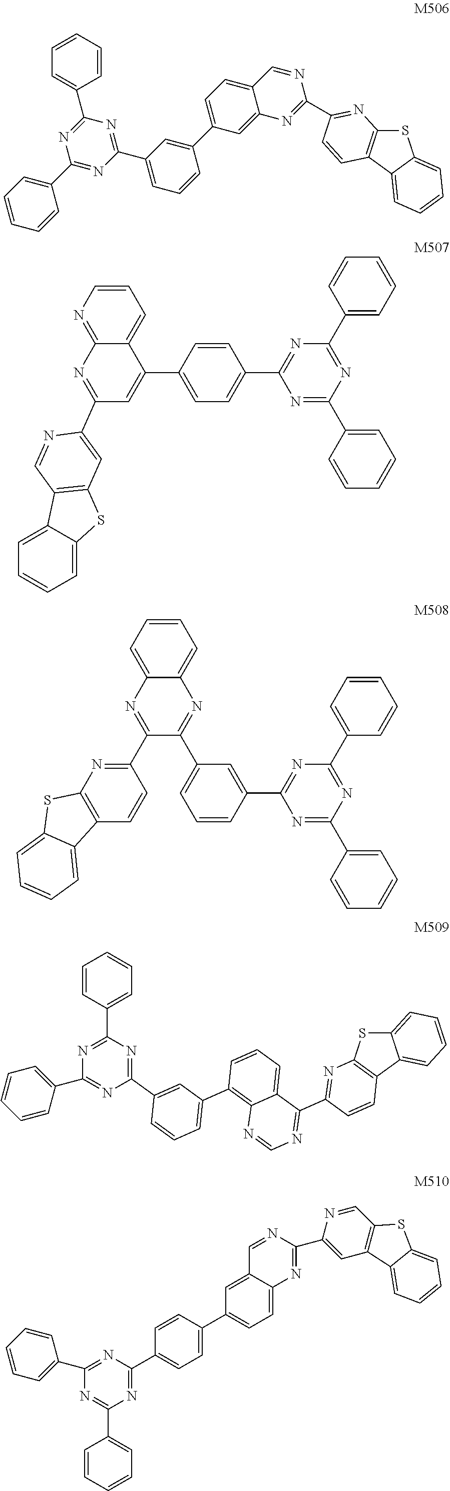 [SMILES: c1ccc(-c2nc(-c3ccccc3)nc(-c3cccc(-c4ccc5cnc(-c6ccc7c(n6)sc6ccccc67)nc5c4)c3)n2)cc1 ; c1ccc(-c2nc(-c3ccccc3)nc(-c3ccc(-c4cc(-c5cc6sc7ccccc7c6cn5)nc5ncccc45)cc3)n2)cc1 ; c1ccc(-c2nc(-c3ccccc3)nc(-c3cccc(-c4nc5ccccc5nc4-c4ccc5c(n4)sc4ccccc45)c3)n2)cc1 ; c1ccc(-c2nc(-c3ccccc3)nc(-c3cccc(-c4cccc5c(-c6ccc7c(n6)sc6ccccc67)ncnc45)c3)n2)cc1 ; c1ccc(-c2nc(-c3ccccc3)nc(-c3ccc(-c4ccc5nc(-c6cc7c(cn6)sc6ccccc67)ncc5c4)cc3)n2)cc1]

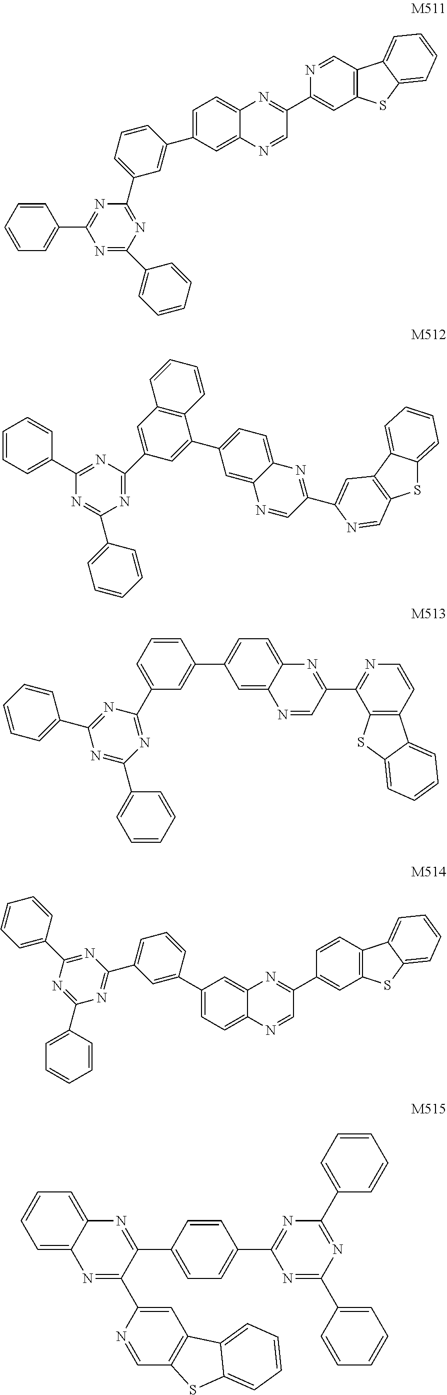 [SMILES: c1ccc(-c2nc(-c3ccccc3)nc(-c3cccc(-c4ccc5nc(-c6cc7sc8ccccc8c7cn6)cnc5c4)c3)n2)cc1 ; c1ccc(-c2nc(-c3ccccc3)nc(-c3cc(-c4ccc5nc(-c6cc7c(cn6)sc6ccccc67)cnc5c4)c4ccccc4c3)n2)cc1 ; c1ccc(-c2nc(-c3ccccc3)nc(-c3cccc(-c4ccc5nc(-c6nccc7c6sc6ccccc67)cnc5c4)c3)n2)cc1 ; c1ccc(-c2nc(-c3ccccc3)nc(-c3cccc(-c4ccc5ncc(-c6ccc7c(c6)sc6ccccc67)nc5c4)c3)n2)cc1 ; c1ccc(-c2nc(-c3ccccc3)nc(-c3ccc(-c4nc5ccccc5nc4-c4cc5c(cn4)sc4ccccc45)cc3)n2)cc1]

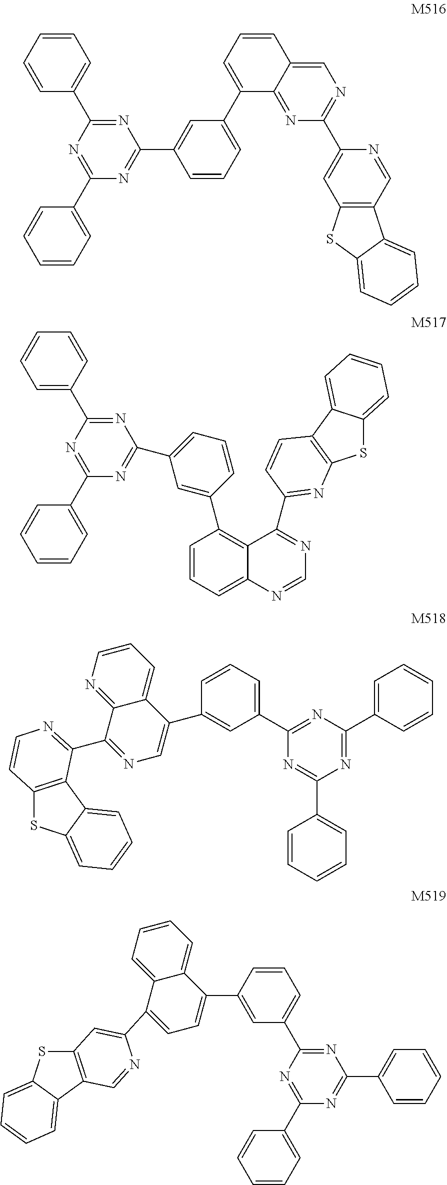 [SMILES: c1ccc(-c2nc(-c3ccccc3)nc(-c3cccc(-c4cccc5cnc(-c6cc7sc8ccccc8c7cn6)nc45)c3)n2)cc1 ; c1ccc(-c2nc(-c3ccccc3)nc(-c3cccc(-c4cccc5ncnc(-c6ccc7c(n6)sc6ccccc67)c45)c3)n2)cc1 ; c1ccc(-c2nc(-c3ccccc3)nc(-c3cccc(-c4cnc(-c5nccc6sc7ccccc7c56)c5ncccc45)c3)n2)cc1 ; c1ccc(-c2nc(-c3ccccc3)nc(-c3cccc(-c4ccc(-c5cc6sc7ccccc7c6cn5)c5ccccc45)c3)n2)cc1]

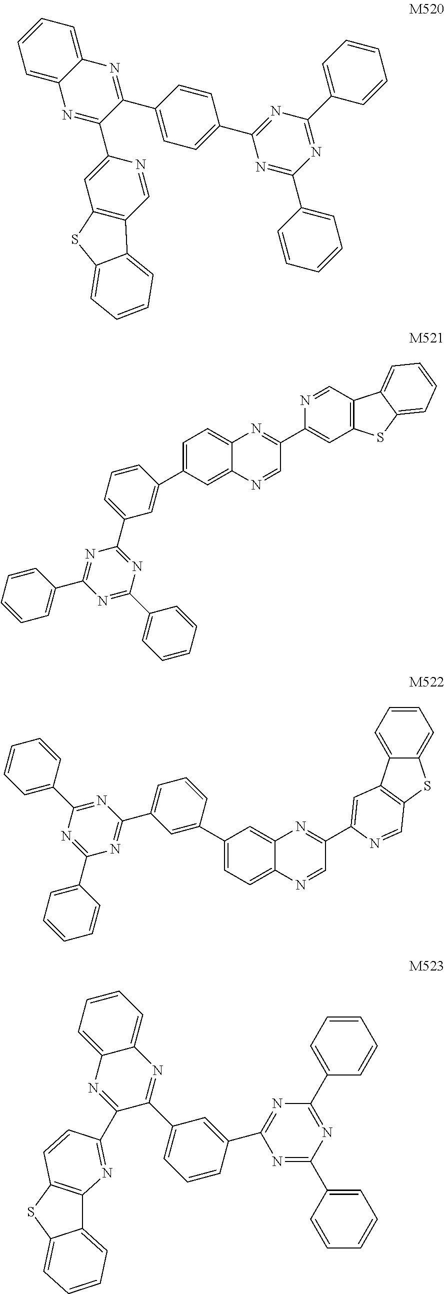 [SMILES: c1ccc(-c2nc(-c3ccccc3)nc(-c3ccc(-c4nc5ccccc5nc4-c4cc5sc6ccccc6c5cn4)cc3)n2)cc1 ; c1ccc(-c2nc(-c3ccccc3)nc(-c3cccc(-c4ccc5nc(-c6cc7sc8ccccc8c7cn6)cnc5c4)c3)n2)cc1 ; c1ccc(-c2nc(-c3ccccc3)nc(-c3cccc(-c4ccc5ncc(-c6cc7c(cn6)sc6ccccc67)nc5c4)c3)n2)cc1 ; c1ccc(-c2nc(-c3ccccc3)nc(-c3cccc(-c4nc5ccccc5nc4-c4ccc5sc6ccccc6c5n4)c3)n2)cc1]

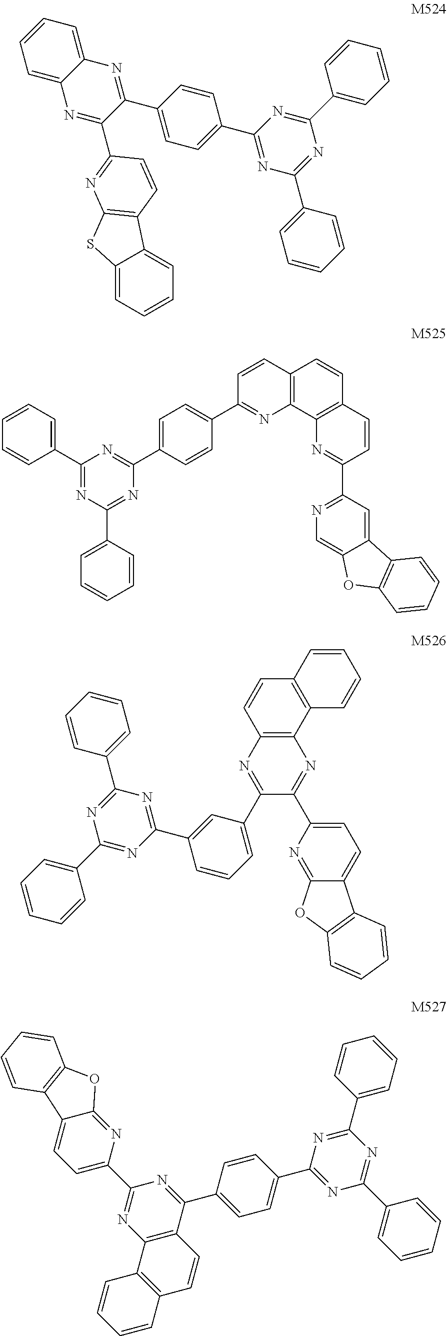 [SMILES: c1ccc(-c2nc(-c3ccccc3)nc(-c3ccc(-c4nc5ccccc5nc4-c4ccc5c(n4)sc4ccccc45)cc3)n2)cc1 ; c1ccc(-c2nc(-c3ccccc3)nc(-c3ccc(-c4ccc5ccc6ccc(-c7cc8c(cn7)oc7ccccc78)nc6c5n4)cc3)n2)cc1 ; c1ccc(-c2nc(-c3ccccc3)nc(-c3cccc(-c4nc5ccc6ccccc6c5nc4-c4ccc5c(n4)oc4ccccc45)c3)n2)cc1 ; c1ccc(-c2nc(-c3ccccc3)nc(-c3ccc(-c4nc(-c5ccc6c(n5)oc5ccccc56)nc5c4ccc4ccccc45)cc3)n2)cc1]

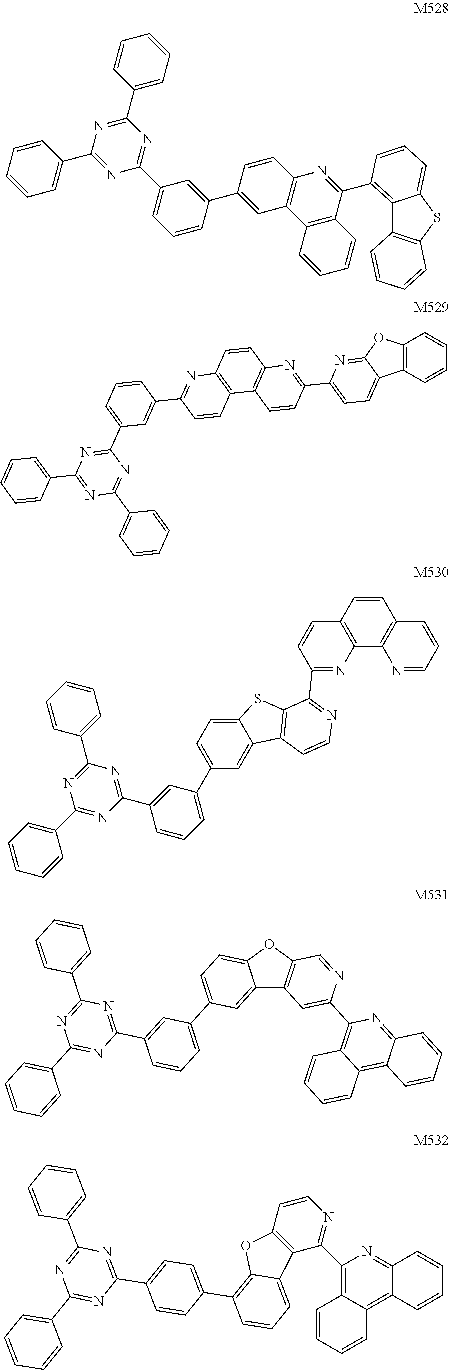 [SMILES: c1ccc(-c2nc(-c3ccccc3)nc(-c3cccc(-c4ccc5nc(-c6cccc7sc8ccccc8c67)c6ccccc6c5c4)c3)n2)cc1 ; c1ccc(-c2nc(-c3ccccc3)nc(-c3cccc(-c4ccc5c(ccc6nc(-c7ccc8c(n7)oc7ccccc78)ccc65)n4)c3)n2)cc1 ; c1ccc(-c2nc(-c3ccccc3)nc(-c3cccc(-c4ccc5sc6c(-c7ccc8ccc9cccnc9c8n7)nccc6c5c4)c3)n2)cc1 ; c1ccc(-c2nc(-c3ccccc3)nc(-c3cccc(-c4ccc5oc6cnc(-c7nc8ccccc8c8ccccc78)cc6c5c4)c3)n2)cc1 ; c1ccc(-c2nc(-c3ccccc3)nc(-c3ccc(-c4cccc5c4oc4ccnc(-c6nc7ccccc7c7ccccc67)c45)cc3)n2)cc1]

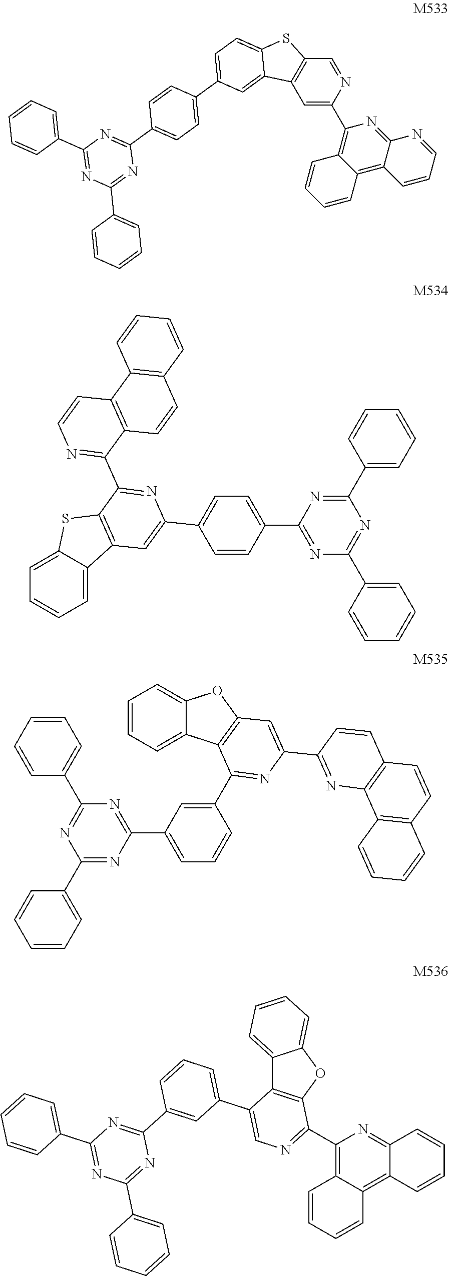 [SMILES: c1ccc(-c2nc(-c3ccccc3)nc(-c3ccc(-c4ccc5sc6cnc(-c7nc8ncccc8c8ccccc78)cc6c5c4)cc3)n2)cc1 ; c1ccc(-c2nc(-c3ccccc3)nc(-c3ccc(-c4cc5c(sc6ccccc65)c(-c5nccc6c5ccc5ccccc56)n4)cc3)n2)cc1 ; c1ccc(-c2nc(-c3ccccc3)nc(-c3cccc(-c4nc(-c5ccc6ccc7ccccc7c6n5)cc5oc6ccccc6c45)c3)n2)cc1 ; c1ccc(-c2nc(-c3ccccc3)nc(-c3cccc(-c4cnc(-c5nc6ccccc6c6ccccc56)c5oc6ccccc6c45)c3)n2)cc1]

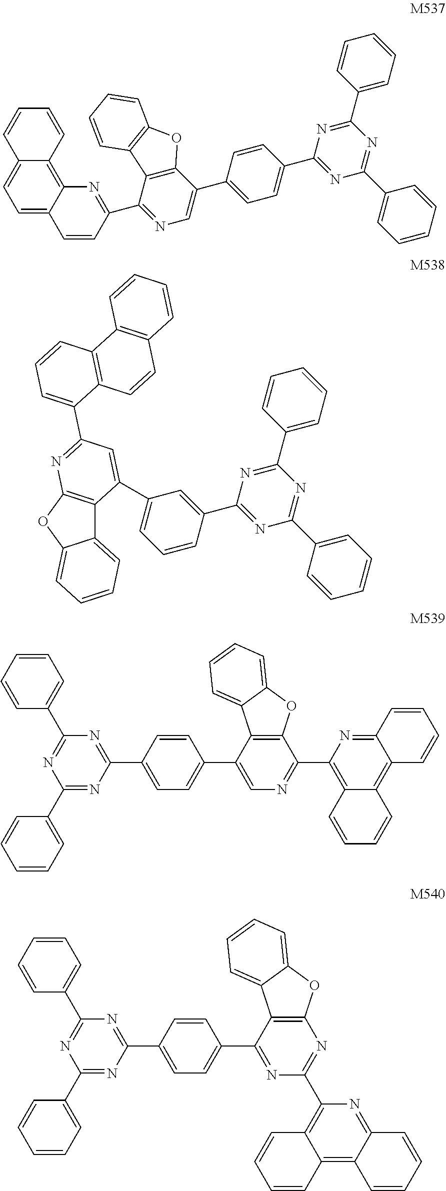 [SMILES: c1ccc(-c2nc(-c3ccccc3)nc(-c3ccc(-c4cnc(-c5ccc6ccc7ccccc7c6n5)c5c4oc4ccccc45)cc3)n2)cc1 ; c1ccc(-c2nc(-c3ccccc3)nc(-c3cccc(-c4cc(-c5cccc6c5ccc5ccccc56)nc5oc6ccccc6c45)c3)n2)cc1 ; c1ccc(-c2nc(-c3ccccc3)nc(-c3ccc(-c4cnc(-c5nc6ccccc6c6ccccc56)c5oc6ccccc6c45)cc3)n2)cc1 ; c1ccc(-c2nc(-c3ccccc3)nc(-c3ccc(-c4nc(-c5nc6ccccc6c6ccccc56)nc5oc6ccccc6c45)cc3)n2)cc1]

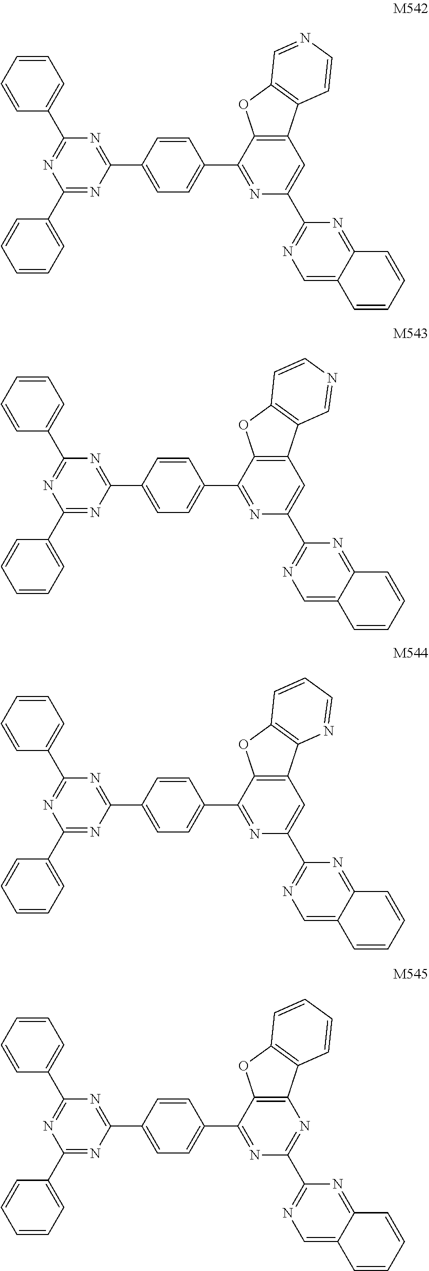 [SMILES: c1ccc(-c2nc(-c3ccccc3)nc(-c3ccc(-c4nc(-c5ncc6ccccc6n5)cc5c4oc4cnccc45)cc3)n2)cc1 ; c1ccc(-c2nc(-c3ccccc3)nc(-c3ccc(-c4nc(-c5ncc6ccccc6n5)cc5c4oc4ccncc45)cc3)n2)cc1 ; c1ccc(-c2nc(-c3ccccc3)nc(-c3ccc(-c4nc(-c5ncc6ccccc6n5)cc5c4oc4cccnc45)cc3)n2)cc1 ; c1ccc(-c2nc(-c3ccccc3)nc(-c3ccc(-c4nc(-c5ncc6ccccc6n5)nc5c4oc4ccccc45)cc3)n2)cc1]

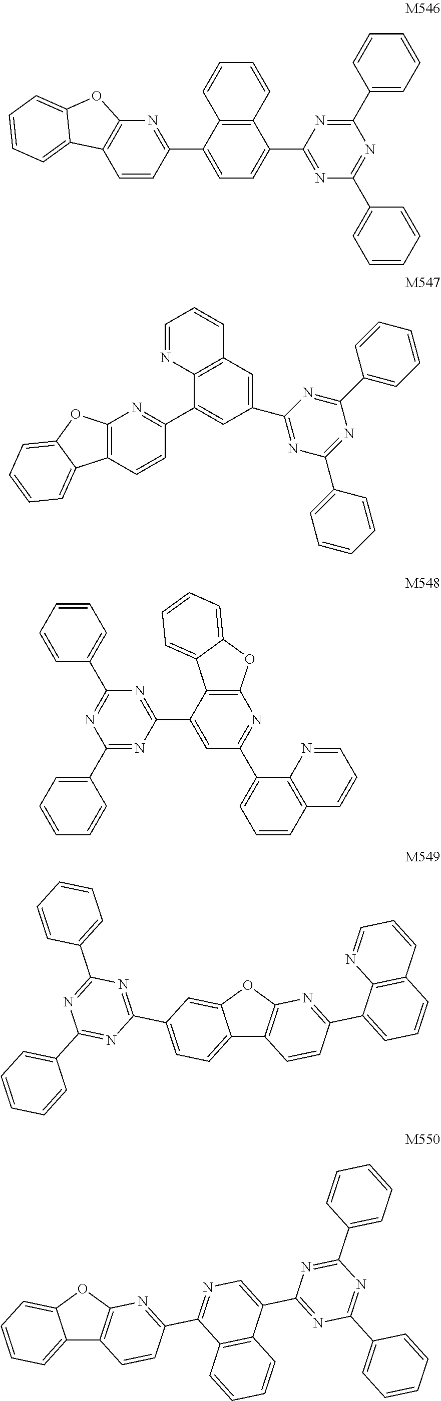 [SMILES: c1ccc(-c2nc(-c3ccccc3)nc(-c3ccc(-c4ccc5c(n4)oc4ccccc45)c4ccccc34)n2)cc1 ; c1ccc(-c2nc(-c3ccccc3)nc(-c3cc(-c4ccc5c(n4)oc4ccccc45)c4ncccc4c3)n2)cc1 ; c1ccc(-c2nc(-c3ccccc3)nc(-c3cc(-c4cccc5cccnc45)nc4oc5ccccc5c34)n2)cc1 ; c1ccc(-c2nc(-c3ccccc3)nc(-c3ccc4c(c3)oc3nc(-c5cccc6cccnc56)ccc34)n2)cc1 ; c1ccc(-c2nc(-c3ccccc3)nc(-c3cnc(-c4ccc5c(n4)oc4ccccc45)c4ccccc34)n2)cc1]

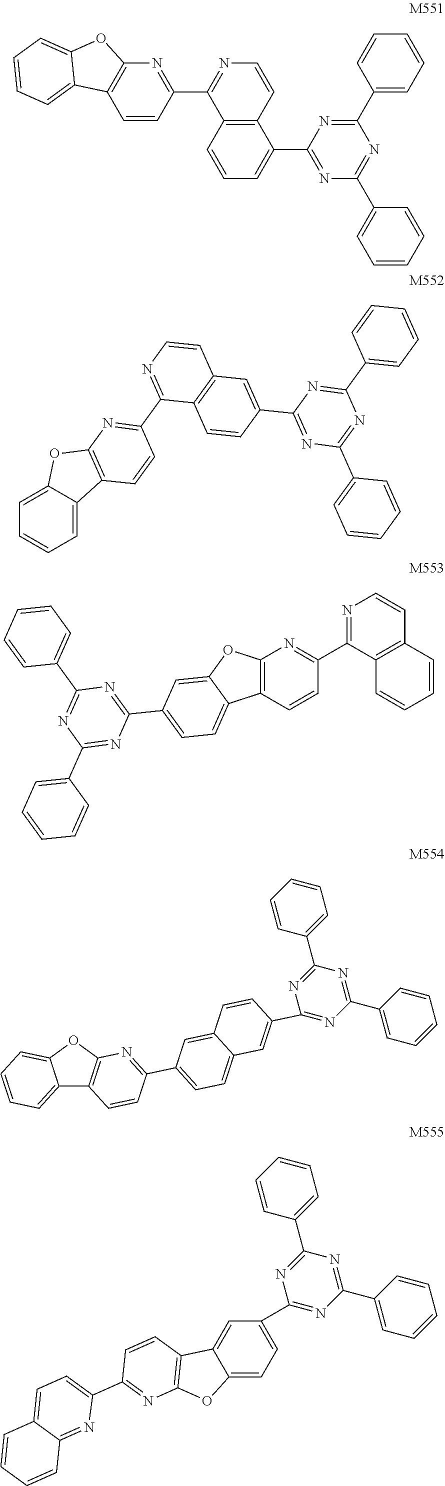

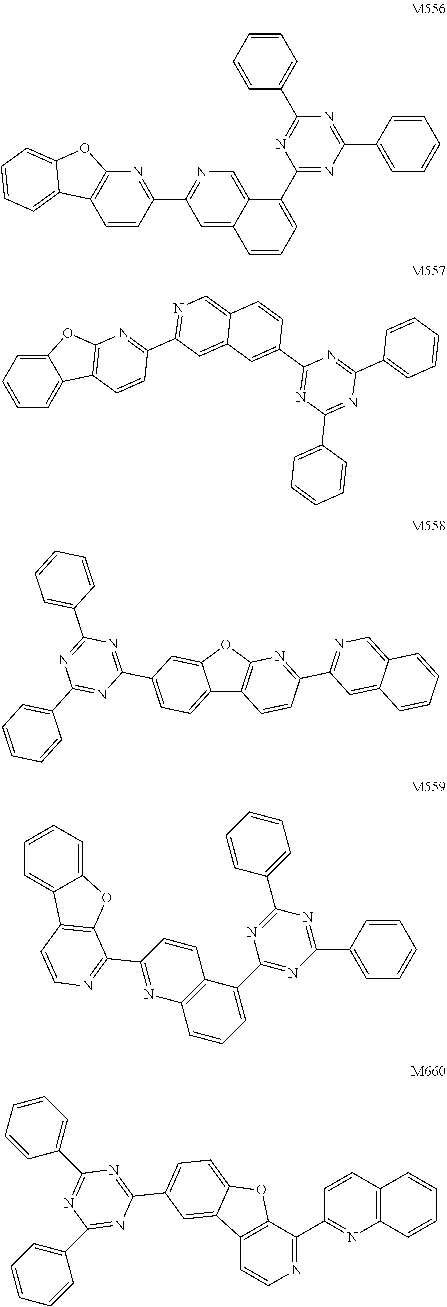 [SMILES: c1ccc(-c2nc(-c3ccccc3)nc(-c3cccc4cc(-c5ccc6c(n5)oc5ccccc56)ncc34)n2)cc1 ; c1ccc(-c2nc(-c3ccccc3)nc(-c3ccc4cnc(-c5ccc6c(n5)oc5ccccc56)cc4c3)n2)cc1 ; c1ccc(-c2nc(-c3ccccc3)nc(-c3ccc4c(c3)oc3nc(-c5cc6ccccc6cn5)ccc34)n2)cc1 ; c1ccc(-c2nc(-c3ccccc3)nc(-c3cccc4nc(-c5nccc6c5oc5ccccc56)ccc34)n2)cc1 ; c1ccc(-c2nc(-c3ccccc3)nc(-c3ccc4oc5c(-c6ccc7ccccc7n6)nccc5c4c3)n2)cc1]

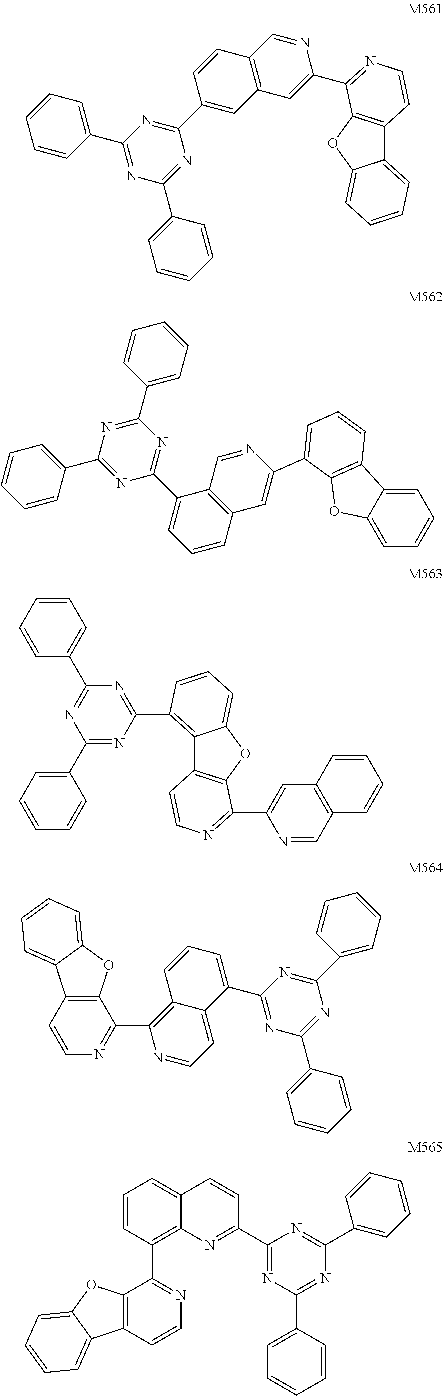 [SMILES: c1ccc(-c2nc(-c3ccccc3)nc(-c3ccc4cnc(-c5nccc6c5oc5ccccc56)cc4c3)n2)cc1 ; c1ccc(-c2nc(-c3ccccc3)nc(-c3cccc4cc(-c5cccc6c5oc5ccccc56)ncc34)n2)cc1 ; c1ccc(-c2nc(-c3ccccc3)nc(-c3cccc4oc5c(-c6cc7ccccc7cn6)nccc5c34)n2)cc1 ; c1ccc(-c2nc(-c3ccccc3)nc(-c3cccc4c(-c5nccc6c5oc5ccccc56)nccc34)n2)cc1 ; c1ccc(-c2nc(-c3ccccc3)nc(-c3ccc4cccc(-c5nccc6c5oc5ccccc56)c4n3)n2)cc1]

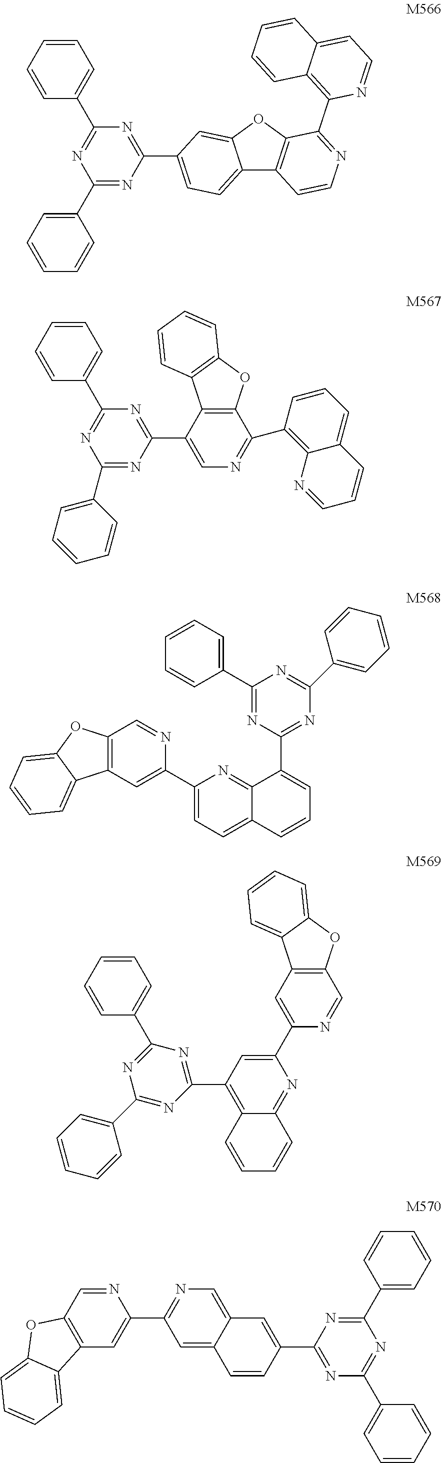

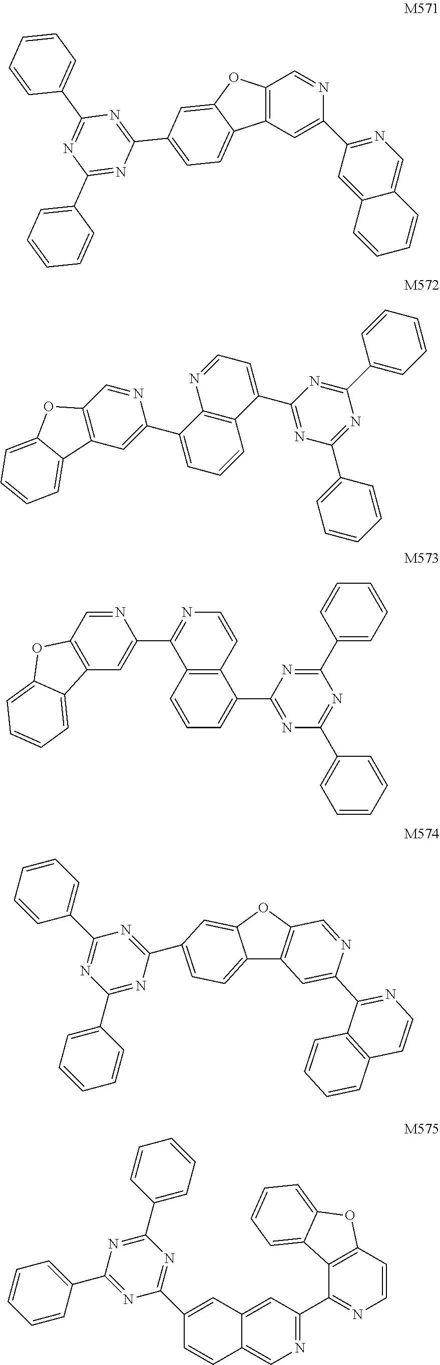

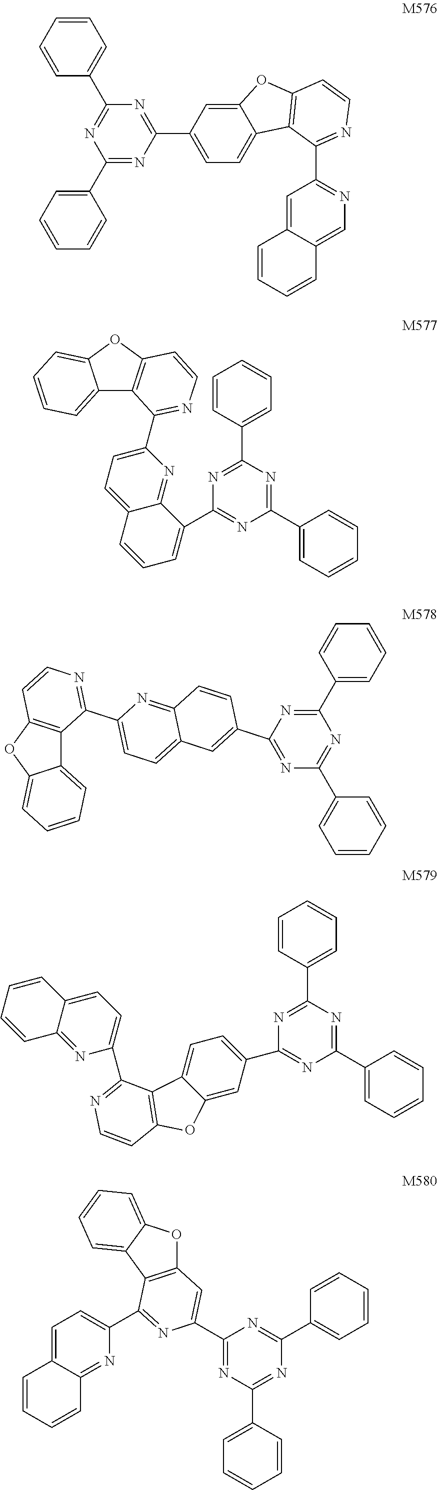 [SMILES: c1ccc(-c2nc(-c3ccccc3)nc(-c3ccc4c(c3)oc3ccnc(-c5cc6ccccc6cn5)c34)n2)cc1 ; c1ccc(-c2nc(-c3ccccc3)nc(-c3cccc4ccc(-c5nccc6oc7ccccc7c56)nc34)n2)cc1 ; c1ccc(-c2nc(-c3ccccc3)nc(-c3ccc4nc(-c5nccc6oc7ccccc7c56)ccc4c3)n2)cc1 ; c1ccc(-c2nc(-c3ccccc3)nc(-c3ccc4c(c3)oc3ccnc(-c5ccc6ccccc6n5)c34)n2)cc1 ; c1ccc(-c2nc(-c3ccccc3)nc(-c3cc4oc5ccccc5c4c(-c4ccc5ccccc5n4)n3)n2)cc1]

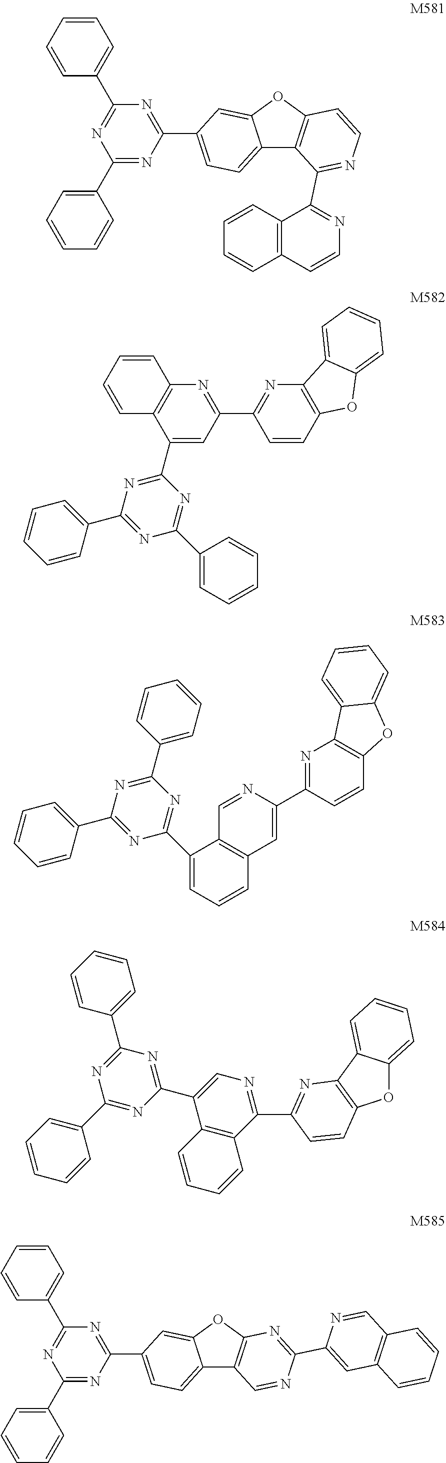 [SMILES: c1ccc(-c2nc(-c3ccccc3)nc(-c3ccc4c(c3)oc3ccnc(-c5nccc6ccccc56)c34)n2)cc1 ; c1ccc(-c2nc(-c3ccccc3)nc(-c3cc(-c4ccc5oc6ccccc6c5n4)nc4ccccc34)n2)cc1 ; c1ccc(-c2nc(-c3ccccc3)nc(-c3cccc4cc(-c5ccc6oc7ccccc7c6n5)ncc34)n2)cc1 ; c1ccc(-c2nc(-c3ccccc3)nc(-c3cnc(-c4ccc5oc6ccccc6c5n4)c4ccccc34)n2)cc1 ; c1ccc(-c2nc(-c3ccccc3)nc(-c3ccc4c(c3)oc3nc(-c5cc6ccccc6cn5)ncc34)n2)cc1]

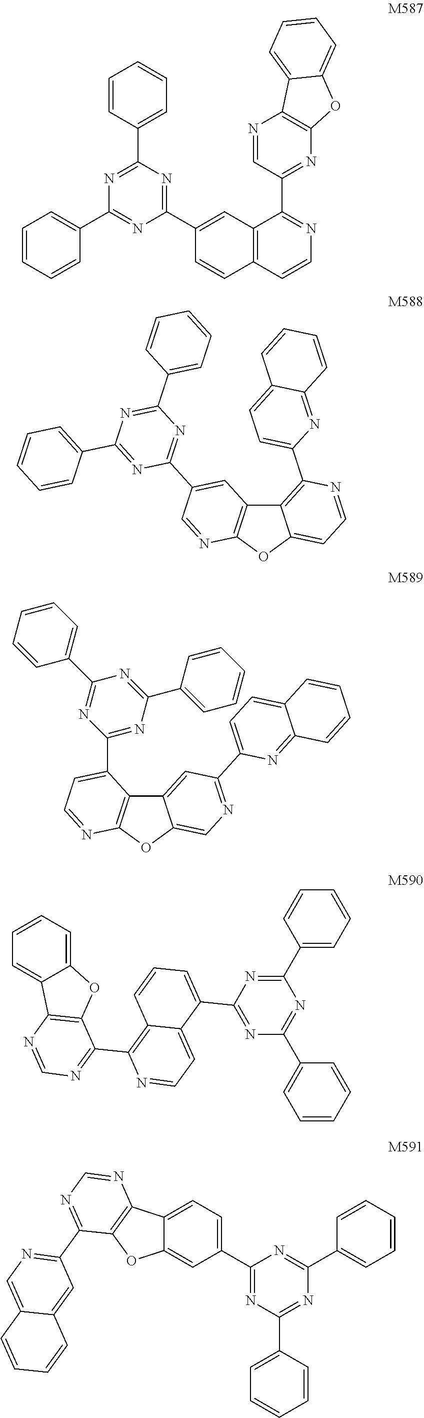 [SMILES: c1ccc(-c2nc(-c3ccccc3)nc(-c3ccc4ccnc(-c5cnc6c(n5)oc5ccccc56)c4c3)n2)cc1 ; c1ccc(-c2nc(-c3ccccc3)nc(-c3cnc4oc5ccnc(-c6ccc7ccccc7n6)c5c4c3)n2)cc1 ; c1ccc(-c2nc(-c3ccccc3)nc(-c3ccnc4oc5cnc(-c6ccc7ccccc7n6)cc5c34)n2)cc1 ; c1ccc(-c2nc(-c3ccccc3)nc(-c3cccc4c(-c5ncnc6c5oc5ccccc56)nccc34)n2)cc1 ; c1ccc(-c2nc(-c3ccccc3)nc(-c3ccc4c(c3)oc3c(-c5cc6ccccc6cn5)ncnc34)n2)cc1]

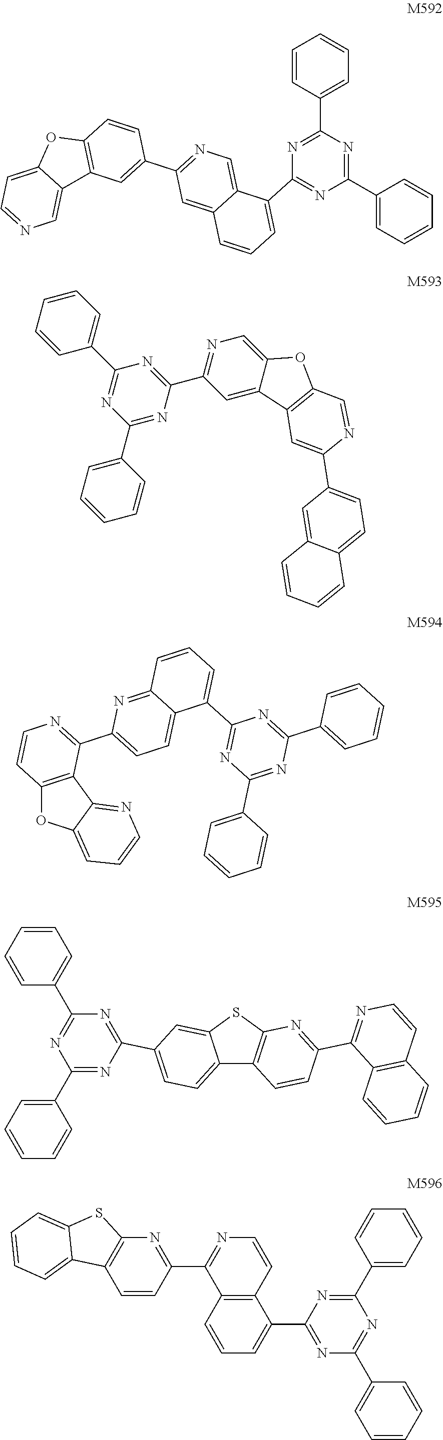 [SMILES: c1ccc(-c2nc(-c3ccccc3)nc(-c3cccc4cc(-c5ccc6oc7ccncc7c6c5)ncc34)n2)cc1 ; c1ccc(-c2nc(-c3ccccc3)nc(-c3cc4c(cn3)oc3cnc(-c5ccc6ccccc6c5)cc34)n2)cc1 ; c1ccc(-c2nc(-c3ccccc3)nc(-c3cccc4nc(-c5nccc6oc7cccnc7c56)ccc34)n2)cc1 ; c1ccc(-c2nc(-c3ccccc3)nc(-c3ccc4c(c3)sc3nc(-c5nccc6ccccc56)ccc34)n2)cc1 ; c1ccc(-c2nc(-c3ccccc3)nc(-c3cccc4c(-c5ccc6c(n5)sc5ccccc56)nccc34)n2)cc1]

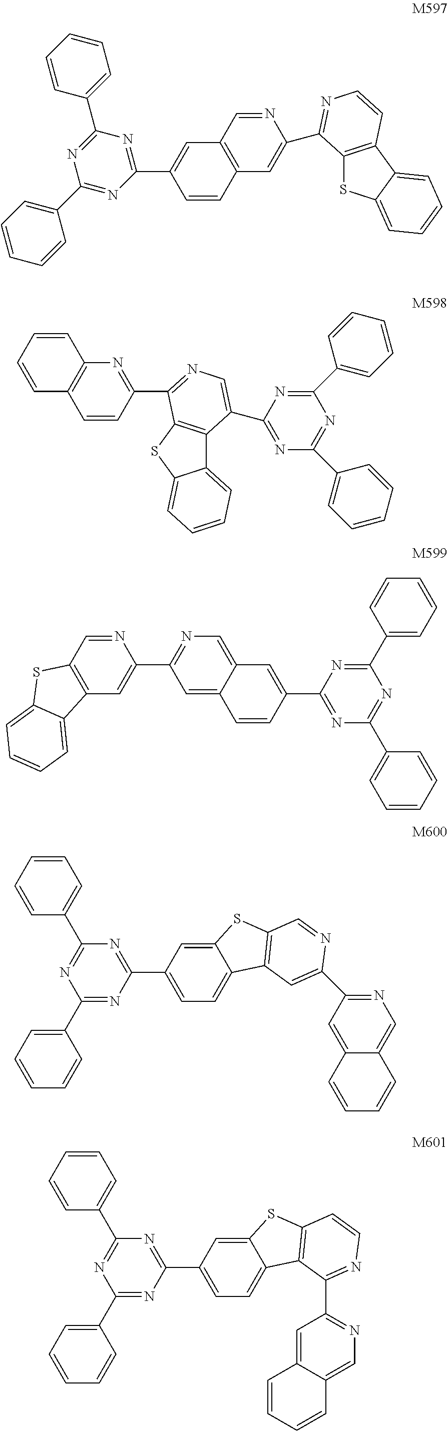 [SMILES: c1ccc(-c2nc(-c3ccccc3)nc(-c3ccc4cc(-c5nccc6c5sc5ccccc56)ncc4c3)n2)cc1 ; c1ccc(-c2nc(-c3ccccc3)nc(-c3cnc(-c4ccc5ccccc5n4)c4sc5ccccc5c34)n2)cc1 ; c1ccc(-c2nc(-c3ccccc3)nc(-c3ccc4cc(-c5cc6c(cn5)sc5ccccc56)ncc4c3)n2)cc1 ; c1ccc(-c2nc(-c3ccccc3)nc(-c3ccc4c(c3)sc3cnc(-c5cc6ccccc6cn5)cc34)n2)cc1 ; c1ccc(-c2nc(-c3ccccc3)nc(-c3ccc4c(c3)sc3ccnc(-c5cc6ccccc6cn5)c34)n2)cc1]

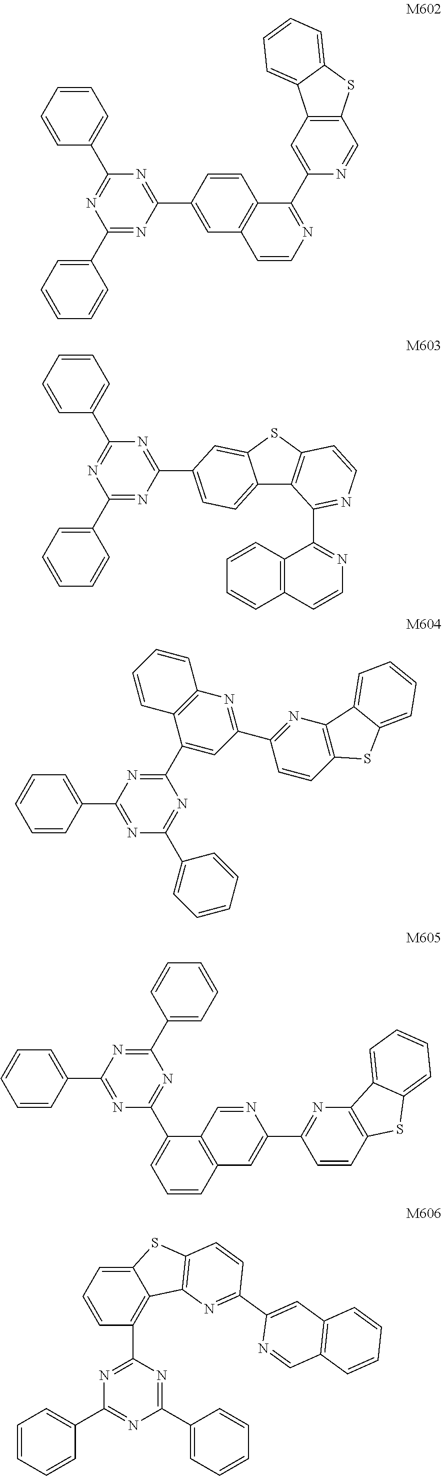

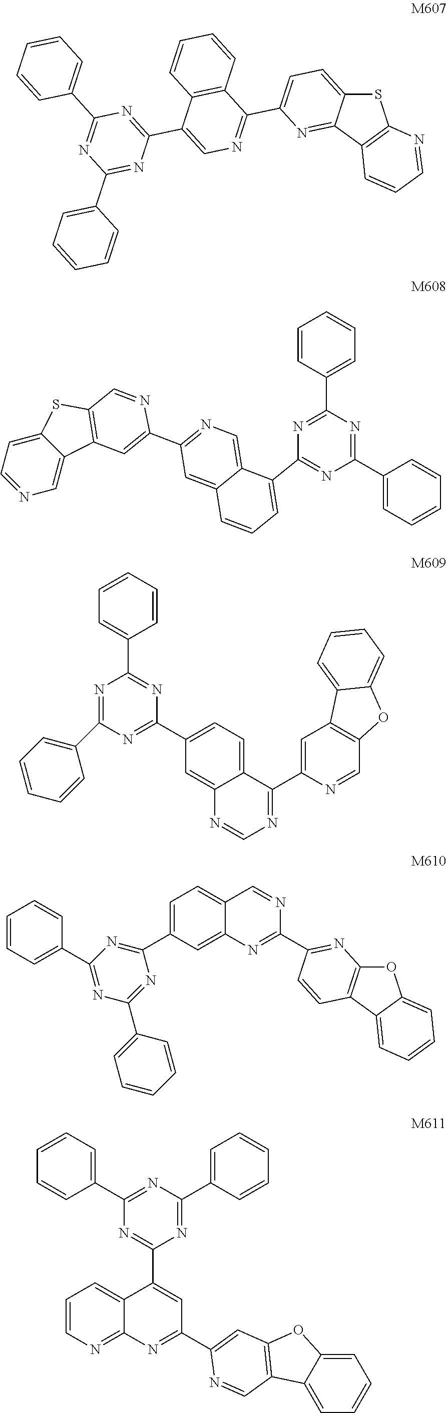 [SMILES: c1ccc(-c2nc(-c3ccccc3)nc(-c3cnc(-c4ccc5sc6ncccc6c5n4)c4ccccc34)n2)cc1 ; c1ccc(-c2nc(-c3ccccc3)nc(-c3cccc4cc(-c5cc6c(cn5)sc5ccncc56)ncc34)n2)cc1 ; c1ccc(-c2nc(-c3ccccc3)nc(-c3ccc4c(-c5cc6c(cn5)oc5ccccc56)ncnc4c3)n2)cc1 ; c1ccc(-c2nc(-c3ccccc3)nc(-c3ccc4cnc(-c5ccc6c(n5)oc5ccccc56)nc4c3)n2)cc1 ; c1ccc(-c2nc(-c3ccccc3)nc(-c3cc(-c4cc5oc6ccccc6c5cn4)nc4ncccc34)n2)cc1]

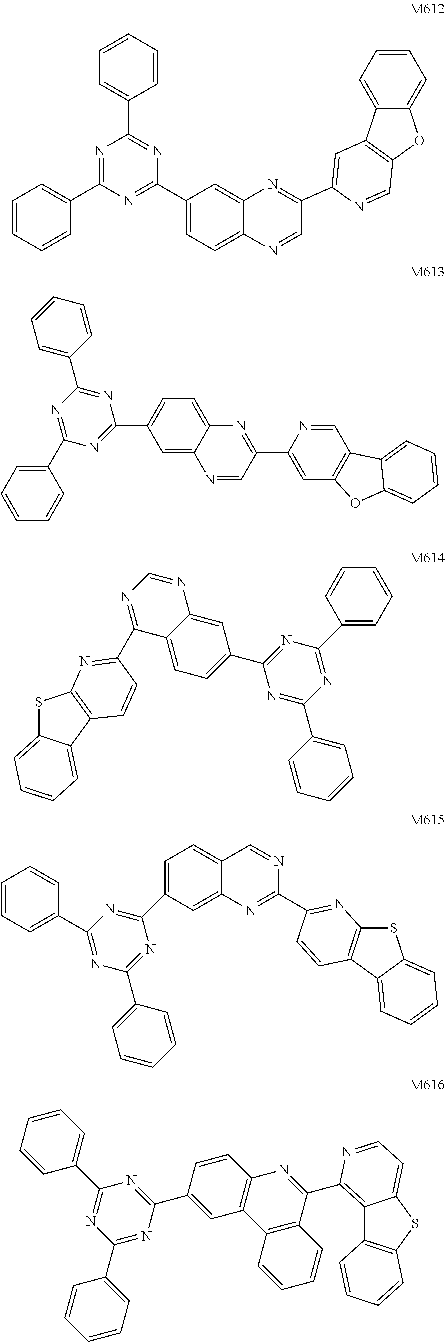 [SMILES: c1ccc(-c2nc(-c3ccccc3)nc(-c3ccc4ncc(-c5cc6c(cn5)oc5ccccc56)nc4c3)n2)cc1 ; c1ccc(-c2nc(-c3ccccc3)nc(-c3ccc4nc(-c5cc6oc7ccccc7c6cn5)cnc4c3)n2)cc1 ; c1ccc(-c2nc(-c3ccccc3)nc(-c3ccc4c(-c5ccc6c(n5)sc5ccccc56)ncnc4c3)n2)cc1 ; c1ccc(-c2nc(-c3ccccc3)nc(-c3ccc4cnc(-c5ccc6c(n5)sc5ccccc56)nc4c3)n2)cc1 ; c1ccc(-c2nc(-c3ccccc3)nc(-c3ccc4nc(-c5nccc6sc7ccccc7c56)c5ccccc5c4c3)n2)cc1]

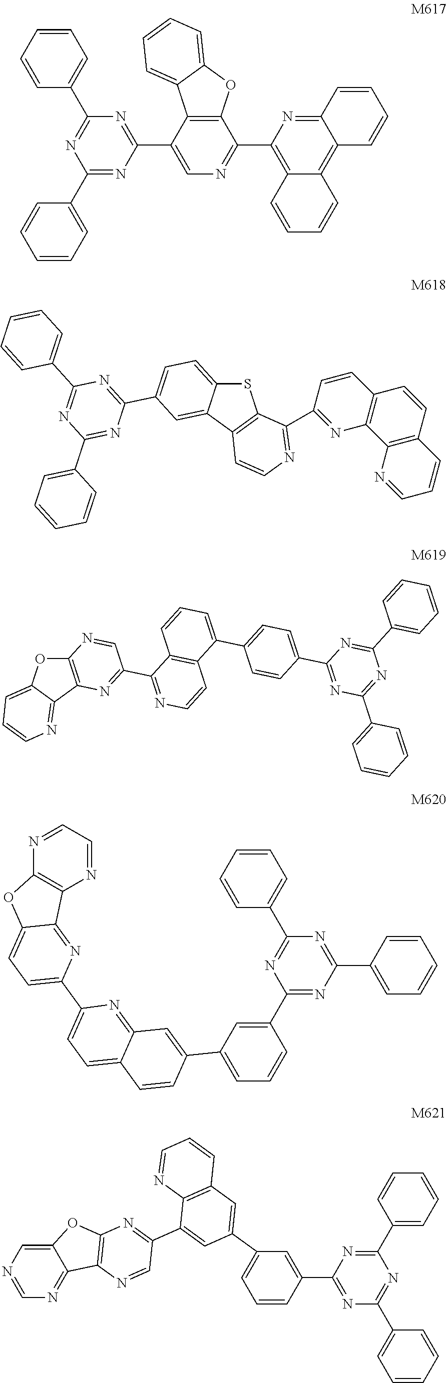 [SMILES: c1ccc(-c2nc(-c3ccccc3)nc(-c3cnc(-c4nc5ccccc5c5ccccc45)c4oc5ccccc5c34)n2)cc1 ; c1ccc(-c2nc(-c3ccccc3)nc(-c3ccc4sc5c(-c6ccc7ccc8cccnc8c7n6)nccc5c4c3)n2)cc1 ; c1ccc(-c2nc(-c3ccccc3)nc(-c3ccc(-c4cccc5c(-c6cnc7oc8cccnc8c7n6)nccc45)cc3)n2)cc1 ; c1ccc(-c2nc(-c3ccccc3)nc(-c3cccc(-c4ccc5ccc(-c6ccc7oc8nccnc8c7n6)nc5c4)c3)n2)cc1 ; c1ccc(-c2nc(-c3ccccc3)nc(-c3cccc(-c4cc(-c5cnc6c(n5)oc5cncnc56)c5ncccc5c4)c3)n2)cc1]

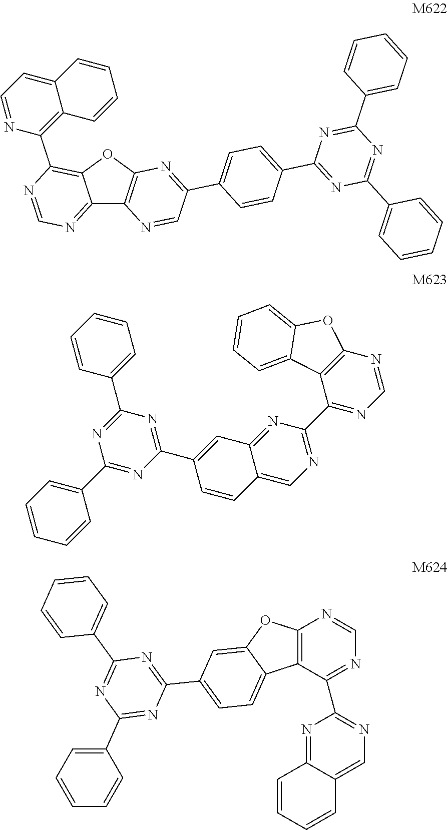

The heterocyclic compounds of the present disclosure are suitable for use in electronic devices, especially in organic electroluminescent devices. Accordingly, the present disclosure further provides use of the compounds of the present disclosure in electronic devices, especially in organic electroluminescent devices.

The present disclosure also provides an organic electroluminescent device comprising an anode, a cathode, and an organic thin film layer located between the anode and the cathode, and the organic thin film layer includes an electron transport layer, and the electron transport layer includes at least one heterocyclic compound represented by formula I.

The present disclosure further provides an organic electroluminescent device comprising an anode, a cathode, an organic thin film layer located between the anode and the cathode, and an organic cover layer located outside the cathode, and the organic cover layer includes at least one heterocyclic compound represented by formula I.

The present disclosure also provides a display panel comprising the above organic electroluminescent device.

The present disclosure also provides a display device comprising the above organic electroluminescent device.

In some embodiments, the first electrode of the organic light-emitting device may be an anode, the second electrode of the organic light-emitting device may be a cathode, and the organic thin film layer of the organic light-emitting device may further include hole transport region between the first electrode and the emitting layer, and the hole transport region may include at least one selected from the group consisting of a hole injection layer, a hole transport layer, an optical auxiliary layer and an electron blocking layer, and the electron transport region may include at least one selected from the group consisting of a hole blocking layer, an electron transport layer and an electron injection layer.

In some embodiments, the electron transport region may include the heterocyclic compound. For example, the electron transport region may include an electron transport layer and an electron injection layer, and at least one selected from the group consisting of the electron transport layer and the electron injection layer may include at least one of the heterocyclic compounds.

In one or more embodiments, at least one layer selected from the group consisting of the electron transport layer and the electron injection layer may further include alkali metals, alkaline earth metals, rare earth metals, alkali metal compounds, alkaline earth metal compounds, rare earth metal compounds, alkali metal complexes, alkaline earth metal complexes, rare earth metal complexes, or any combinations thereof.

In some embodiments, the rare earth metal is Yb.

In some embodiments, the alkali metal complex is LiQ (8-hydroxyquinolinolato-lithium).

The term “organic layer” or “organic thin film layer” as used herein may refer to a single layer or multiple layers between the first electrode and the second electrode of the organic light-emitting device. Materials included in the “organic layer” or “organic thin film layer” are not limited to organic materials.

In other embodiments, the heterocyclic compound represented by formula 1 may be used as a material of a covering layer on the outside of a pair of electrodes of an organic light-emitting device.

The organic light-emitting device of the present disclosure can be prepared by the following methods: forming an anode on a transparent or opaque smooth substrate plate, forming an organic thin layer on the anode, and forming a cathode on the organic thin layer. Among them, the organic thin layer can be formed by using known film forming methods such as vapor deposition, sputtering, spin coating, dipping, and ion plating.

In the organic light-emitting device provided by the present disclosure, the anode material can be metal, metal alloy, metal oxide or conductive polymer; and, the metal includes copper, gold, silver, iron, chromium, nickel, manganese, palladium, platinum and the like, the metal alloy includes an alloy formed by at least two of copper, gold, silver, iron, chromium, nickel, manganese, palladium and platinum, the metal oxide includes indium oxide, zinc oxide, indium tin oxide (ITO), indium zinc oxide (IZO) and the like, the conductive polymer includes polyaniline, polypyrrole, poly(3-methylthiophene) and the like. In addition to the above-described materials and combinations thereof that facilitate hole injection, known materials suitable for use as an anode are also included.

In the organic light-emitting device provided by the present disclosure, the cathode material can be metal, metal alloy or multi-layer metal material; and, the metal includes aluminum, magnesium, silver, indium, tin, titanium and the like, the alloy includes an alloy formed by at least two of aluminum, magnesium, silver, indium, tin and titanium, and the multi-layer metal material includes LiF/A₁, LiO₂/A₁, BaF₂/A₁ and the like. In addition to the above-described materials and combinations thereof that facilitate electron injection, known materials suitable for use as a cathode are also included.

The hole injection layer is a layer that facilitates injection of holes from the anode to the light-emitting layer, and the hole injection substance is a substance that can inject holes well from the anode at a low voltage, preferably the HOMO (highest occupied molecular orbital) of the hole injection substance is between the work function of the anode substance and the HOMO of the surrounding organic layer. Specific examples of the hole injection substance include, but are not limited to, metalloporphyrin-based organic substances, oligothiophene-based organic substances, arylamine-based organic substances, hexacyano hexaazatriphenylene-based organic substances, quinacridone-based organic substances, perylene-based organic substances, anthraquinone, polyaniline and polythiophene-based conductive polymers and the like.

The hole transport layer described above can function to facilitate the transport of holes. The hole transport substance is a substance that can receive holes from the anode or the hole injection layer and transfer them to the light-emitting layer, for which a substance having a high mobility rate of holes is suitable. Specific examples of the hole transport substance include, but are not limited to, arylamine-based organic substances, carbazole-based organic substances, and the like.

The light-emitting layer may emit red, green, or blue light, and may consist of a phosphorescent substance or a fluorescent substance. The light-emitting substance is a substance that can receive holes and electrons from the hole transport layer and the electron transport layer, respectively, and combine them to emit light in the visible light region, and is preferably a substance with high quantum efficiency for fluorescence or phosphorescence. Specific examples of the light-emitting layer include, but are not limited to, 8-hydroxyquinoline aluminum complex (Alq3); carbazole-based compound; dimerized styryl compound; benzofuran, benzothiophene, dibenzofuran, and dibenzothiophene-based compounds; pyrene-based compounds; chrysene-based compounds; boron-nitrogen heterocyclic compounds; benzoxazole, benzothiazole and benzimidazole-based compounds; poly(p-phenylene vinylene) (PPV)-based polymers; spiro compounds; polyfluorene, rubrene, etc.

The host material of the light-emitting layer includes aromatic fused-ring derivatives, heterocycle-containing compounds, and the like. Specifically, the aromatic fused-ring derivatives include, but are not limited to, naphthalene-based compounds, anthrylene-based compounds, phenanthrylene-based compounds, chrysene-based compounds, pyrene-based compounds, pentacene-based compounds, fluoranthene-based compounds and the like, and the heterocycle-containing compounds include, but are not limited to, aromatic amine-based compounds, carbazole-based compounds, dibenzofuran-based compounds, dibenzothiophene-based compounds and the like.

When the light-emitting layer emits red light and green light, the light-emitting doping agent includes, but is not limited to, phosphorescent substances such as Ir metal complexes, Pt metal complexes, and Pd metal complexes, or fluorescent substances such as Alq3. When the light-emitting layer emits blue light, the light-emitting doping agent includes, but is not limited to, phosphorescent substances such as Ir metal complexes, Pt metal complexes, and Pd metal complexes, or fluorescent substances such as spiro-DPVBi, spiro-6P, distyrylbenzene (DSB), distyrylarylene (DSA), PFO-based polymers, PPV-based polymers, chrysene-based compounds, pyrene-based compounds, and boron-nitrogen heterocyclic compounds.

The above-mentioned hole blocking layer is a layer that prevents holes from reaching the cathode, and can be formed under the same condition as the hole injection layer in general. Specifically, the material of the hole blocking layer includes, but is not limited to, oxadiazole-based compounds, triazine-based compounds, phenanthroline-based compounds, BCP, Bphen, aluminum complexes, and the like.

In order to further illustrate the present disclosure, the heterocyclic compound and the organic electroluminescent device provided by the present disclosure will be described in detail below with reference to the examples.

The representative synthetic route of the compound of formula I provided by the present disclosure is as follows:

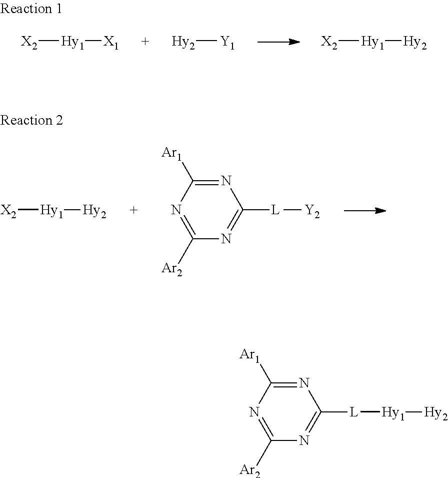

X₁, X₂ are independently selected from the group consisting of F, C₁, Br, I;

Y₁, Y₂ are independently selected from the group consisting of Sn(Me)₃, B(OH)₂,

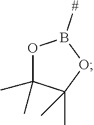

### Preparation Example 1 Synthesis of Compound M013

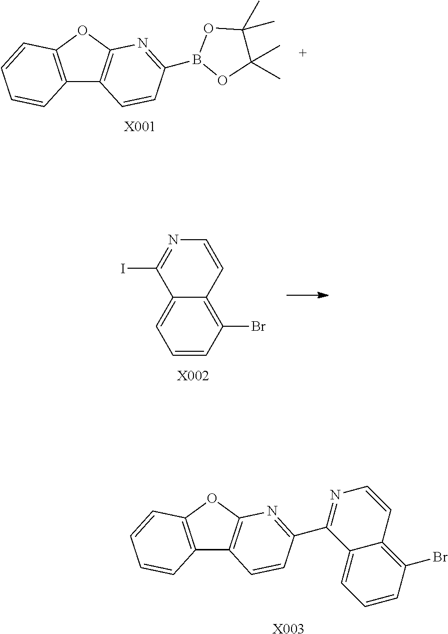

Under the protection of nitrogen, compounds X001 (5.2 mmol), X002 (5.0 mmol), [Pd₂(dba)₃]·CHCl₃ (250 μmol), and HP(tBu)₃·BF₄ (500 μmol) were weighed, and added to a 250 mL two-necked flask. 150 mL of THF (in which N₂ had been introduced for 15 min in advance to remove oxygen) was injected into the two-necked flask, then 15 mL of K₂CO₃ aqueous solution with a concentration of 1M (in which N₂ had been introduced for 15 min in advance to remove oxygen) was added dropwise to the flask, and the obtained mixture was refluxed and stirred overnight. After the reaction was completed, the solution was concentrated to 50 mL, then added with 100 mL of deionized water, and added with a few drops of 2 M HCl dropwise. The obtained solution was extracted with dichloromethane, and the organic phase was collected, and dried over anhydrous Na₂SO₄. The dried solution was filtered and removed off the solvent with a rotary evaporator to obtain a crude product. The crude product was filtered through a short silica gel column to remove the catalyst and inorganic salt impurities in the crude product. After the filtrate was collected and concentrated, the concentrated filtrate was subjected to column chromatography using dichloromethane/n-hexane as the eluent to obtain the intermediate X003 (4.25 mmol, 85% yield).

MALDI-TOF-MS(m/z): calcd for C₁₂₆H₁₂₄N₂O₂S₂₀:2336.4; found: 2336.6;

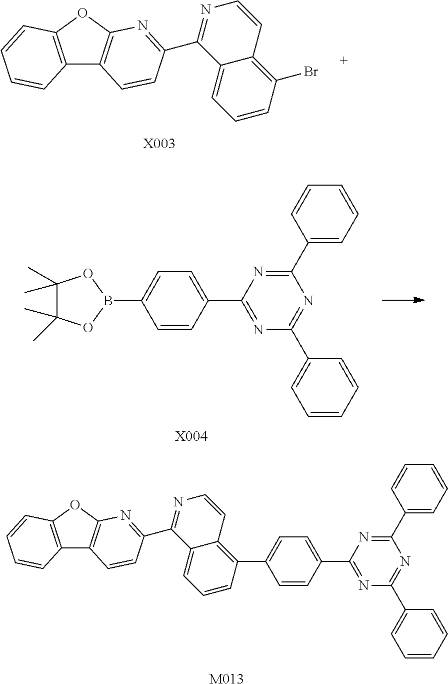 [SMILES: Brc1cccc2c(-c3ccc4c(n3)oc3ccccc34)nccc12 ; "X003" ; CC1(C)OB(c2ccc(-c3nc(-c4ccccc4)nc(-c4ccccc4)n3)cc2)OC1(C)C ; "X004" ; c1ccc(-c2nc(-c3ccccc3)nc(-c3ccc(-c4cccc5c(-c6ccc7c(n6)oc6ccccc67)nccc45)cc3)n2)cc1 ; "M013"]

Under the protection of nitrogen, compounds X₀₀₃(5.0 mmol), X₀₀₄(5.2 mmol), [Pd₂(dba)₃]·CHCl₃ (250 μmol), and HP(tBu)₃·BF₄ (500 μmol) were weighed, and added to a 500 mL two-necked flask. 200 mL of THF (in which N₂ had been introduced for 15 min in advance to remove oxygen) was injected into the two-necked flask, then 15 mL of K₂CO₃ aqueous solution with a concentration of 1M (in which N₂ had been introduced for 15 min in advance to remove oxygen) was added dropwise to the flask, and the obtained mixture was refluxed and stirred overnight. After the reaction was completed, the solution was concentrated to 50 mL, then added with 100 mL of deionized water, and added with a few drops of 2 M HCl dropwise. The obtained solution was extracted with dichloromethane, and the organic phase was collected, and dried over anhydrous Na₂SO₄. The dried solution was filtered and removed off the solvent with a rotary evaporator to obtain a crude product. The crude product was filtered through a short silica gel column to remove the catalyst and inorganic salt impurities in the crude product. After the filtrate was collected and concentrated, the concentrated filtrate was subjected to column chromatography using dichloromethane/n-hexane as the eluent to obtain the target product M013 (3.80 mmol, 76% yield).

### Preparation Example 2

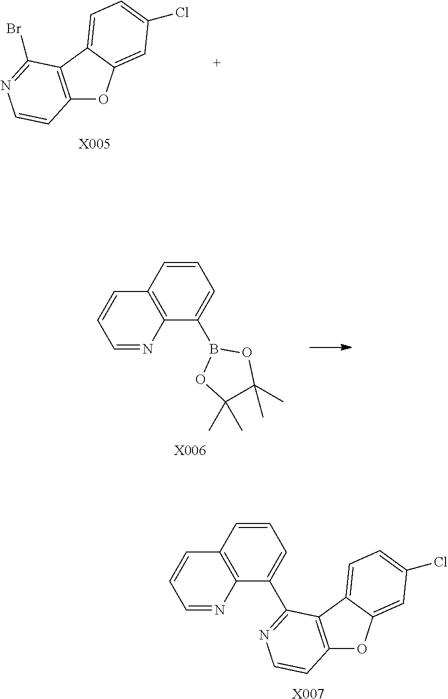

Intermediate X007 (4.10 mmol, 82% yield) was obtained by substantially the same procedure as the synthesis of X003 in Preparation Example 1, except that X005 (5.0 mmol) and X006 (5.2 mmol) were used to replace X002 and X001, respectively.

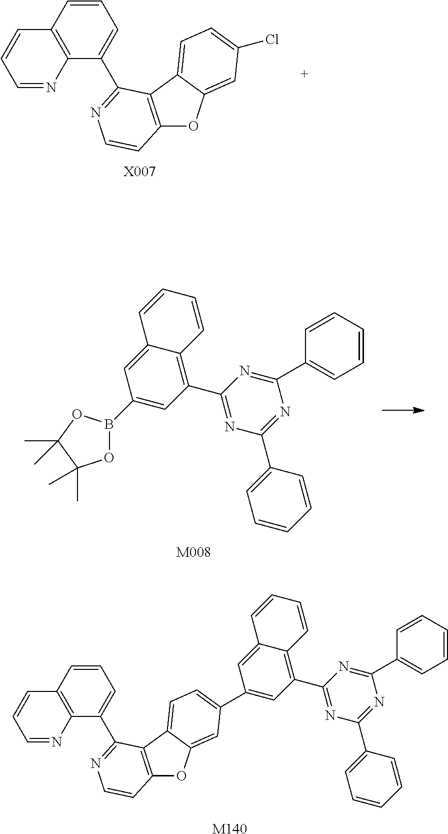

Target compound M140 (3.70 mmol, 74% yield) was obtained by substantially the same procedure as the synthesis of M013 in Preparation Example 1, except that X007 (5.0 mmol) and X008 (5.2 mmol) were used to replace X003 and X004, respectively.

### Preparation Example 3

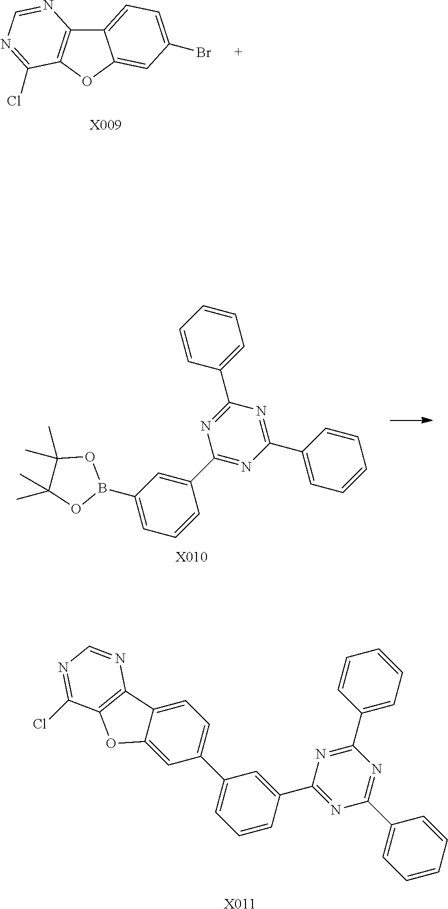

Intermediate X011 (4.15 mmol, 83% yield) was obtained by substantially the same procedure as the synthesis of X003 in Preparation Example 1, except that X009 (5.0 mmol) and X010 (5.2 mmol) were used to replace X002 and X001, respectively.

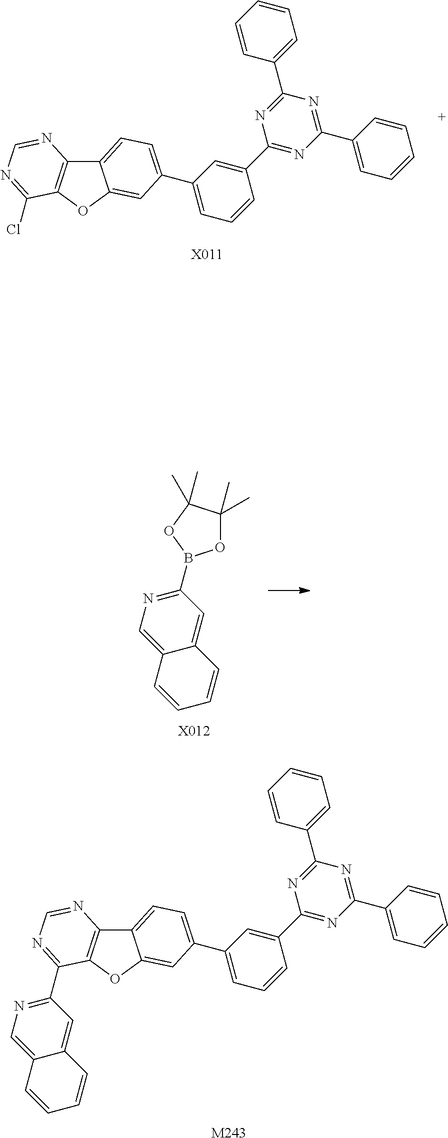

Target compound M243 (3.95 mmol, 79% yield) was obtained by substantially the same procedure as the synthesis of M013 in Preparation Example 1, except that X011 (5.0 mmol) and X012 (5.2 mmol) were used to replace X003 and X004, respectively.

### Preparation Example 4

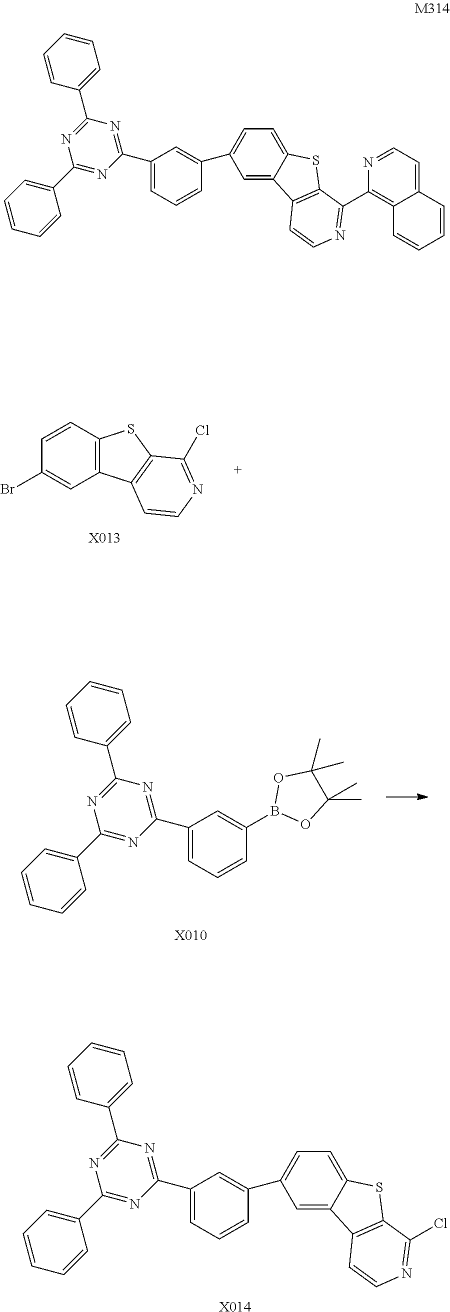

Intermediate X014 (4.0 mmol, 80% yield) was obtained by substantially the same procedure as the synthesis of X003 in Preparation Example 1, except that X013 (5.0 mmol) and X010 (5.2 mmol) were used to replace X002 and X001, respectively.

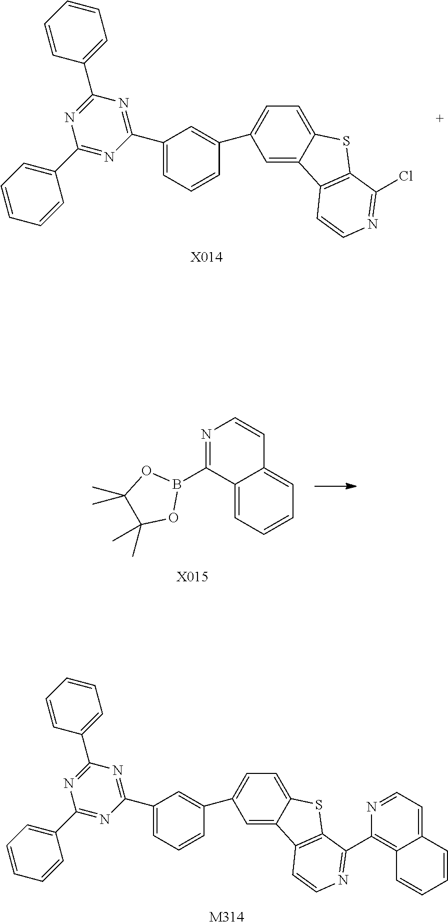 [SMILES: Clc1nccc2c1sc1ccc(-c3cccc(-c4nc(-c5ccccc5)nc(-c5ccccc5)n4)c3)cc12 ; "X014" ; CC1(C)OB(c2nccc3ccccc23)OC1(C)C ; "X015" ; c1ccc(-c2nc(-c3ccccc3)nc(-c3cccc(-c4ccc5sc6c(-c7nccc8ccccc78)nccc6c5c4)c3)n2)cc1 ; "M314"]

Target compound M314 (3.9 mmol, 78% yield) was obtained by substantially the same procedure as the synthesis of M013 in Preparation Example 1, except that X014 (5.0 mmol) and X015 (5.2 mmol) were used to replace X003 and X004, respectively.

### Preparation Example 5

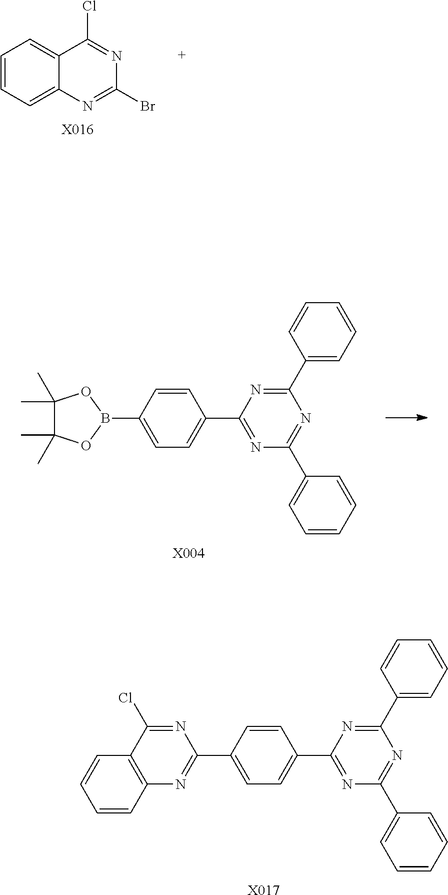

Intermediate X017 (4.05 mmol, 81% yield) was obtained by substantially the same procedure as the synthesis of X003 in Preparation Example 1, except that X016 (5.0 mmol) and X004 (5.2 mmol) were used to replace X002 and X001, respectively.

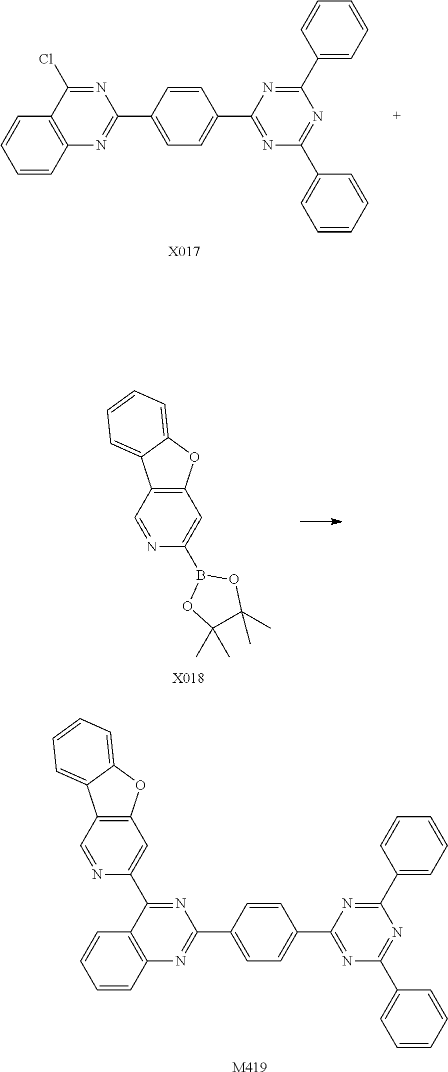

Target compound M419 (3.85 mmol, 77% yield) was obtained by substantially the same procedure as the synthesis of M013 in Preparation Example 1, except that X017 (5.0 mmol) and X018 (5.2 mmol) were used to replace X003 and X004, respectively.

### Example 6

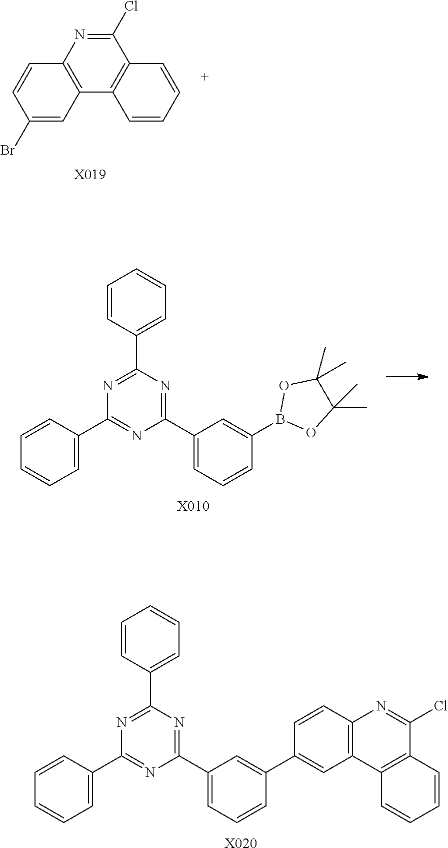

Intermediate X020 (3.95 mmol, 79% yield) was obtained by substantially the same procedure as the synthesis of X003 in Preparation Example 1, except that X019 (5.0 mmol) and X010 (5.2 mmol) were used to replace X002 and X001, respectively.

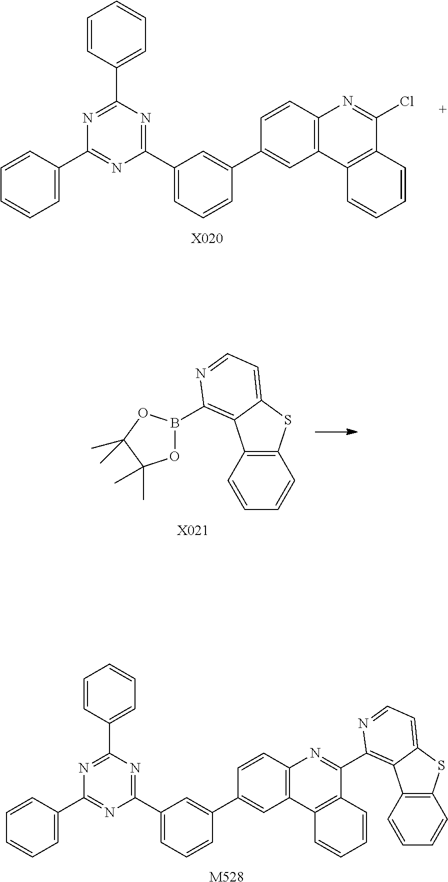

Target compound M528 (3.6 mmol, 72% yield) was obtained by substantially the same procedure as the synthesis of M013 in Preparation Example 1, except that X020 (5.0 mmol) and X021 (5.2 mmol) were used to replace X003 and X004, respectively.

### Example 7

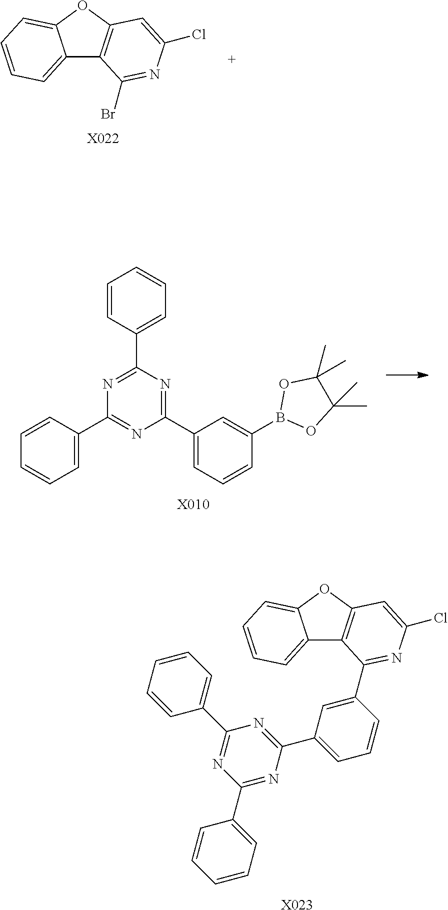

Intermediate X023 (4.0 mmol, 80% yield) was obtained by substantially the same procedure as the synthesis of X003 in Preparation Example 1, except that X022 (5.0 mmol) and X010 (5.2 mmol) were used to replace X002 and X001, respectively.

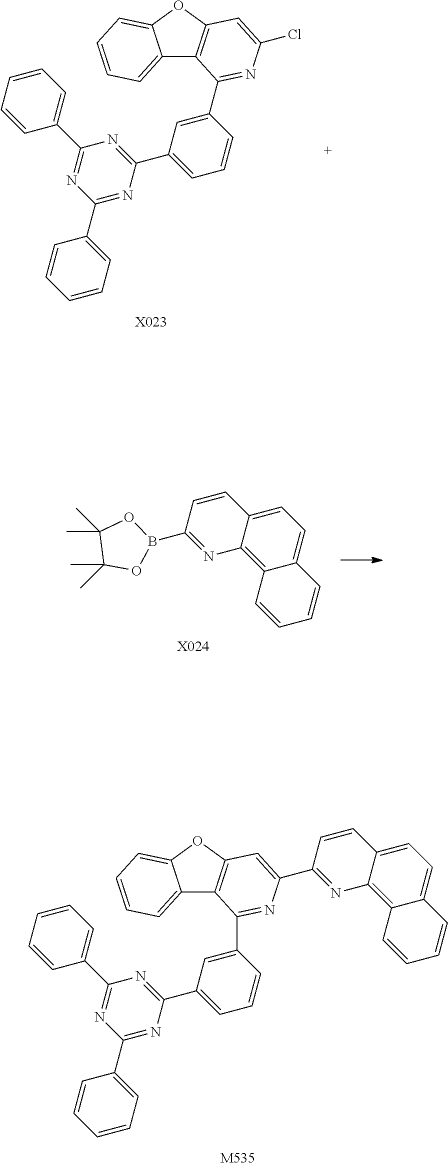 [SMILES: Clc1cc2oc3ccccc3c2c(-c2cccc(-c3nc(-c4ccccc4)nc(-c4ccccc4)n3)c2)n1 ; "X023" ; CC1(C)OB(c2ccc3ccc4ccccc4c3n2)OC1(C)C ; "X024" ; c1ccc(-c2nc(-c3ccccc3)nc(-c3cccc(-c4nc(-c5ccc6ccc7ccccc7c6n5)cc5oc6ccccc6c45)c3)n2)cc1 ; "M535"]

Target compound M535 (3.5 mmol, 70% yield) was obtained by substantially the same procedure as the synthesis of M013 in Preparation Example 1, except that X023 (5.0 mmol) and X024 (5.2 mmol) were used to replace X003 and X004, respectively.

The mass spectrometry and elemental analysis data of the above compounds are shown in Table 1:

TABLE 1

| | | Molecular | |
|---|---|---|---|
| Compound | Chemical Structure | weight | Elemental analysis |
| M001 | 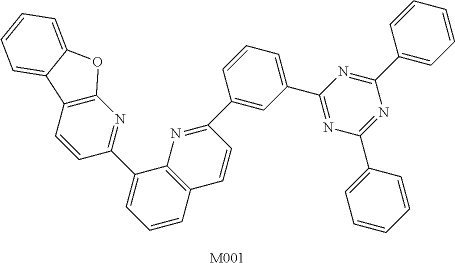 [SMILES: c1ccc(-c2nc(-c3ccccc3)nc(-c3cccc(-c4ccc5cccc(-c6ccc7c(n6)oc6ccccc67)c5n4)c3)n2)cc1 ; "M001"] | Theoretical value 603.2; Measured value 603.3 | Theoretical value: C, 81.57; H, 4.17; N, 11.60; Measured value C, 81.62; H, 4.13; N, 11.52 |
| M008 | 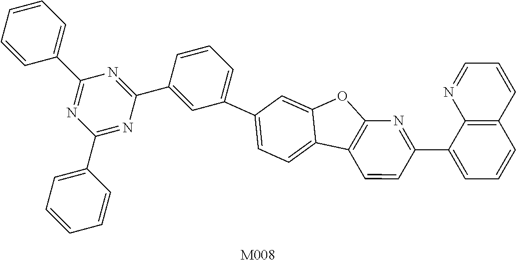 [SMILES: c1ccc(-c2nc(-c3ccccc3)nc(-c3cccc(-c4ccc5c(c4)oc4nc(-c6cccc7cccnc67)ccc45)c3)n2)cc1 ; "M008"] | Theoretical value 603.2; Measured value 603.2 | Theoretical value: C, 81.57; H, 4.17; N, 11.60; Measured value C, 81.62; H, 4.12; N, 11.50; |
| M013 | 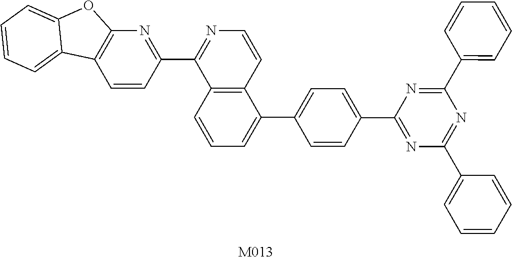 [SMILES: c1ccc(-c2nc(-c3ccccc3)nc(-c3ccc(-c4cccc5c(-c6ccc7c(n6)oc6ccccc67)nccc45)cc3)n2)cc1 ; "M013"] | Theoretical value 603.2; Measured value 603.4 | Theoretical value: C, 81.57; H, 4.17; N, 11.60; Measured value C, 81.63; H, 4.12; N, 11.51 |
| M025 | 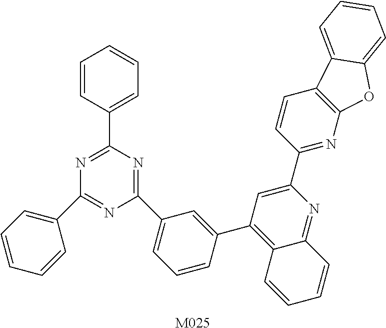 [SMILES: c1ccc(-c2nc(-c3ccccc3)nc(-c3cccc(-c4cc(-c5ccc6c(n5)oc5ccccc56)nc5ccccc45)c3)n2)cc1 ; "M025"] | Theoretical value 603.2; Measured value 603.5 | Theoretical value: C, 81.57; H, 4.17; N, 11.60; Measured value C, 81.60; H, 4.09; N, 11.49; |
| M037 | 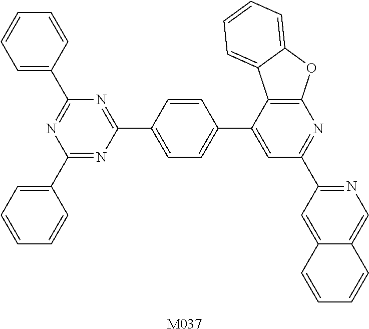 [SMILES: c1ccc(-c2nc(-c3ccccc3)nc(-c3ccc(-c4cc(-c5cc6ccccc6cn5)nc5oc6ccccc6c45)cc3)n2)cc1 ; "M037"] | Theoretical value 603.2; Measured value 603.1 | Theoretical value: C, 81.57; H, 4.17; N, 11.60; Measured value C, 81.67; H, 4.08; N, 11.46; |
| M049 | 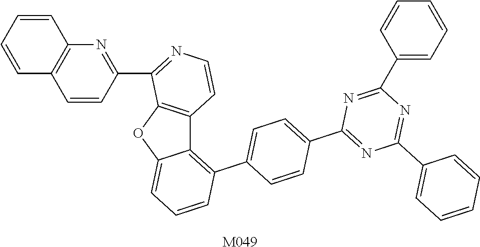 | Theoretical value 603.2; Measured value 603.3 | Theoretical value: C, 81.57; H, 4.17; N, 11.60; Measured value C, 81.66; H, 4.10; N, 11.55; |
| M061 | 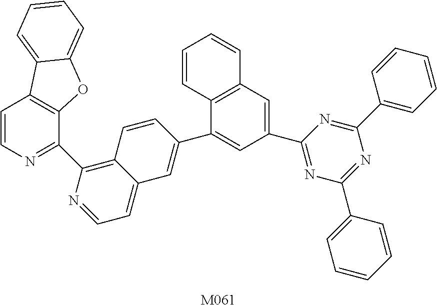 [SMILES: c1ccc(-c2nc(-c3ccccc3)nc(-c3cc(-c4ccc5c(-c6nccc7c6oc6ccccc67)nccc5c4)c4ccccc4c3)n2)cc1 ; "M061"] | Theoretical value 653.2; Measured value 653.8 | Theoretical value: C, 82.68; H, 4.16; N, 10.71; Measured value C, 82.75; H, 4.07; N, 10.60; |
| M073 | 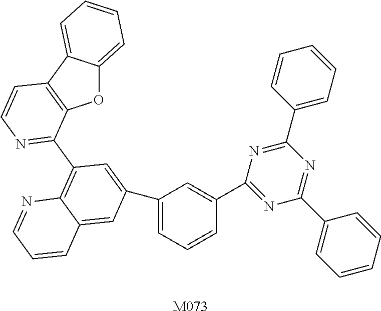 [SMILES: c1ccc(-c2nc(-c3ccccc3)nc(-c3cccc(-c4cc(-c5nccc6c5oc5ccccc56)c5ncccc5c4)c3)n2)cc1 ; "M073"] | Theoretical value 603.2; Measured value 603.5 | Theoretical value: C, 81.57; H, 4.17; N, 11.60; Measured value C, 81.64; H, 4.12; N, 11.51; |
| M083 | 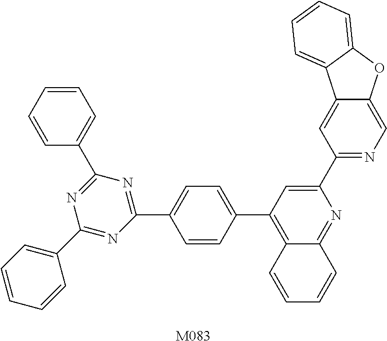 [SMILES: c1ccc(-c2nc(-c3ccccc3)nc(-c3ccc(-c4cc(-c5cc6c(cn5)oc5ccccc56)nc5ccccc45)cc3)n2)cc1 ; "M083"] | Theoretical value 603.2; Measured value 603.0 | Theoretical value: C, 81.57; H, 4.17; N, 11.60; Measured value C, 81.60; H, 4.11; N, 11.53; |
| M086 | 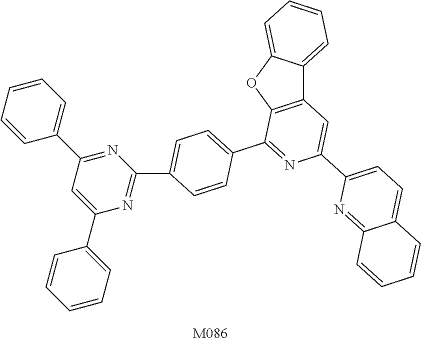 [SMILES: c1ccc(-c2cc(-c3ccccc3)nc(-c3ccc(-c4nc(-c5ccc6ccccc6n5)cc5c4oc4ccccc45)cc3)n2)cc1 ; "M086"] | Theoretical value 603.2; Measured value 603.2 | Theoretical value: C, 81.57; H, 4.17; N, 11.60; Measured value C, 81.59; H, 4.14; N, 11.54; |
| M088 | 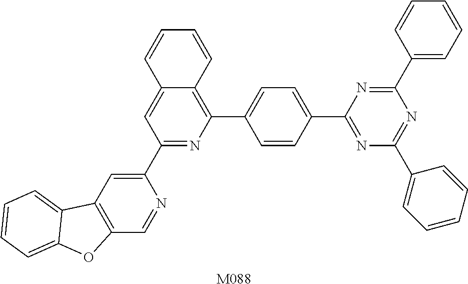 [SMILES: c1ccc(-c2nc(-c3ccccc3)nc(-c3ccc(-c4nc(-c5cc6c(cn5)oc5ccccc56)cc5ccccc45)cc3)n2)cc1 ; "M088"] | Theoretical value 603.2; Measured value 603.3 | Theoretical value: C, 81.57; H, 4.17; N, 11.60; Measured value C, 81.63; H, 4.11; N, 11.57; |
| M096 | 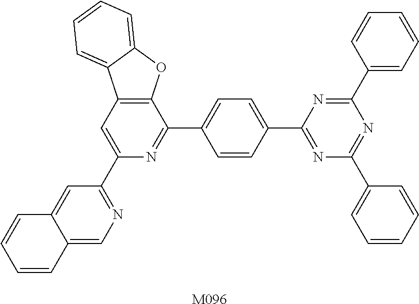 [SMILES: c1ccc(-c2nc(-c3ccccc3)nc(-c3ccc(-c4nc(-c5cc6ccccc6cn5)cc5c4oc4ccccc45)cc3)n2)cc1 ; "M096"] | Theoretical value 603.2; Measured value 603.5 | Theoretical value: C, 81.57; H, 4.17; N, 11.60; Measured value C, 81.66; H, 4.12; N, 11.49; |
| M103 | 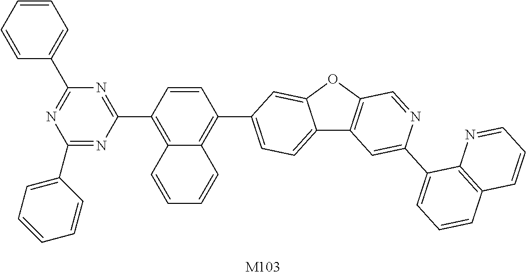 [SMILES: c1ccc(-c2nc(-c3ccccc3)nc(-c3ccc(-c4ccc5c(c4)oc4cnc(-c6cccc7cccnc67)cc45)c4ccccc34)n2)cc1 ; "M103"] | Theoretical value 653.2; Measured value 653.3 | Theoretical value: C, 82.68; H, 4.16; N, 10.71; Measured value C, 82.73; H, 4.10; N, 10.64; |
| M110 | 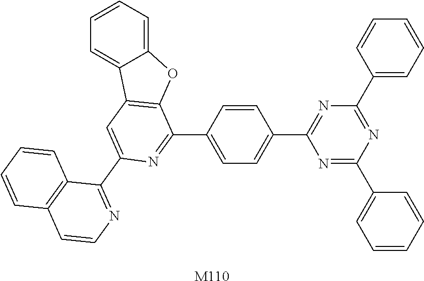 [SMILES: c1ccc(-c2nc(-c3ccccc3)nc(-c3ccc(-c4nc(-c5nccc6ccccc56)cc5c4oc4ccccc45)cc3)n2)cc1 ; "M110"] | Theoretical value 603.2; Measured value 603.3 | Theoretical value: C, 81.57; H, 4.17; N, 11.60; Measured value C, 81.63; H, 4.14; N, 11.53; |
| M121 | 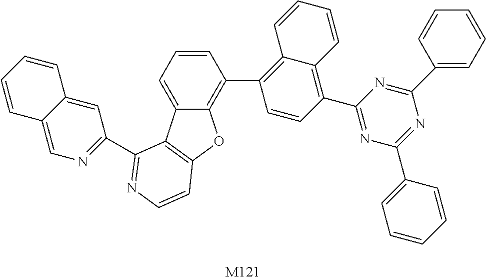 [SMILES: c1ccc(-c2nc(-c3ccccc3)nc(-c3ccc(-c4cccc5c4oc4ccnc(-c6cc7ccccc7cn6)c45)c4ccccc34)n2)cc1 ; "M121"] | Theoretical value 653.2; Measured value 653.7 | Theoretical value: C, 82.68; H, 4.16; N, 10.71; Measured value C, 82.77; H, 4.09; N, 10.68; |
| M133 | 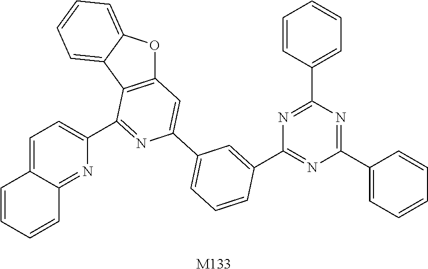 [SMILES: c1ccc(-c2nc(-c3ccccc3)nc(-c3cccc(-c4cc5oc6ccccc6c5c(-c5ccc6ccccc6n5)n4)c3)n2)cc1 ; "M133"] | Theoretical value 603.2; Measured value 603.6 | Theoretical value: C, 81.57; H, 4.17; N, 11.60; Measured value C, 81.65; H, 4.11; N, 11.55; |
| M140 | 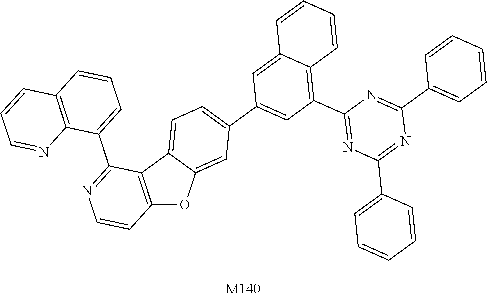 [SMILES: c1ccc(-c2nc(-c3ccccc3)nc(-c3cc(-c4ccc5c(c4)oc4ccnc(-c6cccc7cccnc67)c45)cc4ccccc34)n2)cc1 ; "M140"] | Theoretical value 653.2; Measured value 653.9 | Theoretical value: C, 82.68; H, 4.16; N, 10.71; Measured value C, 82.75; H, 4.10; N, 10.67; |
| M151 | 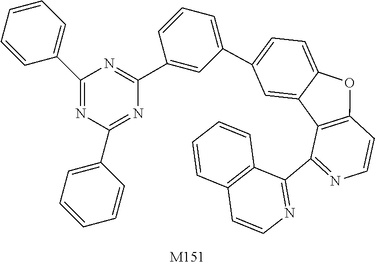 [SMILES: c1ccc(-c2nc(-c3ccccc3)nc(-c3cccc(-c4ccc5oc6ccnc(-c7nccc8ccccc78)c6c5c4)c3)n2)cc1 ; "M151"] | Theoretical value 603.2; Measured value 603.2 | Theoretical value: C, 81.57; H, 4.17; N, 11.60; Measured value C, 81.68; H, 4.08; N, 11.54; |
| M159 | 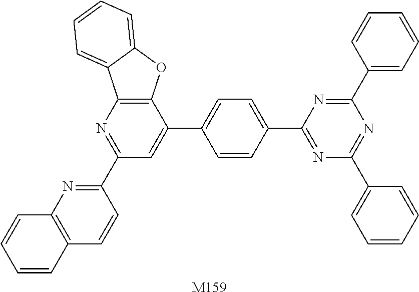 [SMILES: c1ccc(-c2nc(-c3ccccc3)nc(-c3ccc(-c4cc(-c5ccc6ccccc6n5)nc5c4oc4ccccc45)cc3)n2)cc1 ; "M159"] | Theoretical value 603.2; Measured value 603.1 | Theoretical value: C, 81.57; H, 4.17; N, 11.60; Measured value C, 81.64; H, 4.09; N, 11.52; |
| M169 | 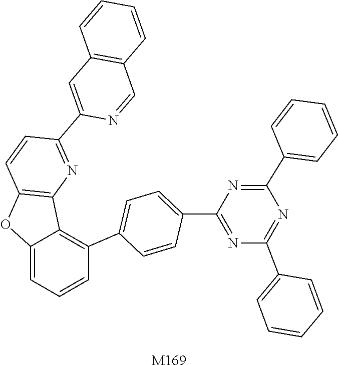 [SMILES: c1ccc(-c2nc(-c3ccccc3)nc(-c3ccc(-c4cccc5oc6ccc(-c7cc8ccccc8cn7)nc6c45)cc3)n2)cc1 ; "M169"] | Theoretical value 603.2; Measured value 603.3 | Theoretical value: C, 81.57; H, 4.17; N, 11.60; Measured value C, 81.60; H, 4.12; N, 11.54; |
| M181 | 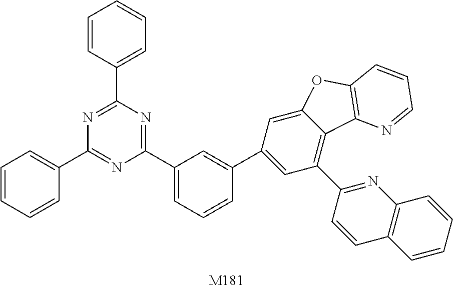 [SMILES: c1ccc(-c2nc(-c3ccccc3)nc(-c3cccc(-c4cc(-c5ccc6ccccc6n5)c5c(c4)oc4cccnc45)c3)n2)cc1 ; "M181"] | Theoretical value 603.2; Measured value 603.2 | Theoretical value: C, 81.57; H, 4.17; N, 11.60; Measured value C, 81.62; H, 4.13; N, 11.56; |
| M193 | 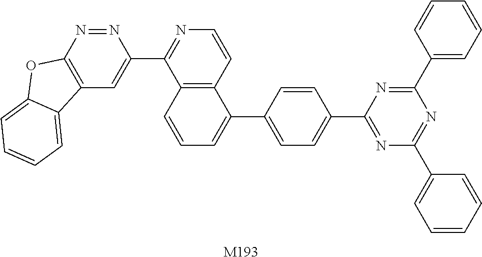 [SMILES: c1ccc(-c2nc(-c3ccccc3)nc(-c3ccc(-c4cccc5c(-c6cc7c(nn6)oc6ccccc67)nccc45)cc3)n2)cc1 ; "M193"] | Theoretical value 604.2; Measured value 604.5 | Theoretical value: C, 79.45; H, 4.00; N, 13.90; Measured value C, 79.53; H, 3.95; N, 13.84; |
| M205 | 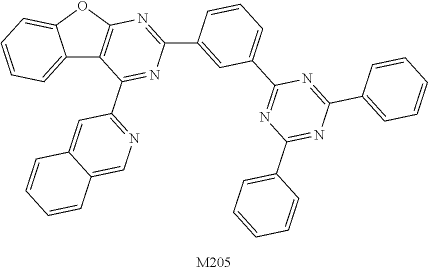 [SMILES: c1ccc(-c2nc(-c3ccccc3)nc(-c3cccc(-c4nc(-c5cc6ccccc6cn5)c5c(n4)oc4ccccc45)c3)n2)cc1 ; "M205"] | Theoretical value 604.2; Measured value 604.7 | Theoretical value: C, 79.45; H, 4.00; N, 13.90; Measured value C, 79.52; H, 3.93; N, 13.86; |
| M217 | 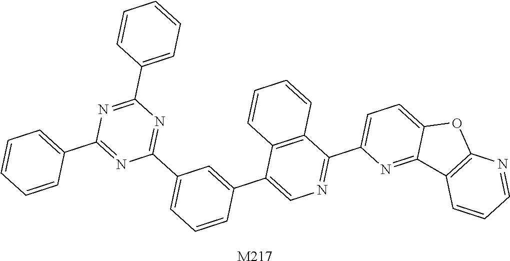 | Theoretical value 604.2; Measured value 604.0 | Theoretical value: C, 79.45; H, 4.00; N, 13.90; Measured value C, 79.54; H, 3.92; N, 13.80 |
| M229 | 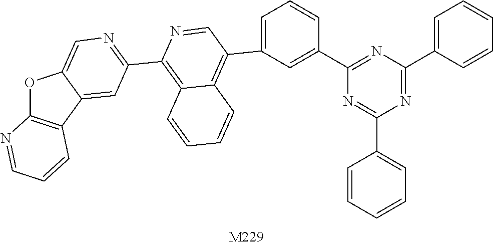 [SMILES: c1ccc(-c2nc(-c3ccccc3)nc(-c3cccc(-c4cnc(-c5cc6c(cn5)oc5ncccc56)c5ccccc45)c3)n2)cc1 ; "M229"] | Theoretical value 604.2; Measured value 604.4 | Theoretical value: C, 79.45; H, 4.00; N, 13.90; Measured value C, 79.50; H, 3.95; N, 13.82; |
| M243 | 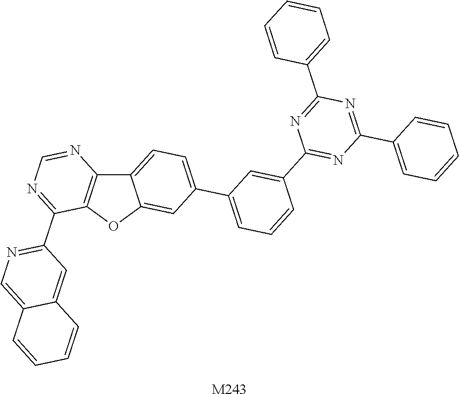 [SMILES: c1ccc(-c2nc(-c3ccccc3)nc(-c3cccc(-c4ccc5c(c4)oc4c(-c6cc7ccccc7cn6)ncnc45)c3)n2)cc1 ; "M243"] | Theoretical value 604.2; Measured value 604.2 | Theoretical value: C, 79.45; H, 4.00; N, 13.90 Measured value C, 79.57; H, 3.90; N, 13.83 |
| M253 | 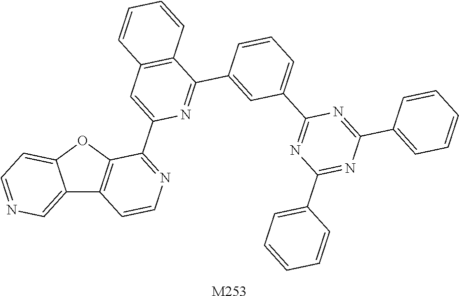 [SMILES: c1ccc(-c2nc(-c3ccccc3)nc(-c3cccc(-c4nc(-c5nccc6c5oc5ccncc56)cc5ccccc45)c3)n2)cc1 ; "M253"] | Theoretical value 604.2; Measured value 604.4 | Theoretical value: C, 79.45; H, 4.00; N, 13.90; Measured value C, 79.51; H, 3.97; N, 13.86; |
| M277 | 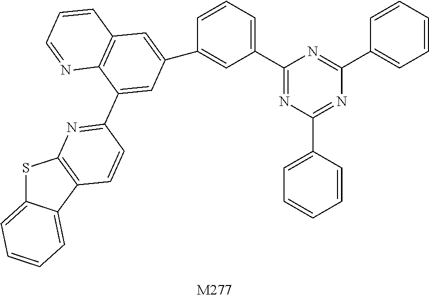 [SMILES: c1ccc(-c2nc(-c3ccccc3)nc(-c3cccc(-c4cc(-c5ccc6c(n5)sc5ccccc56)c5ncccc5c4)c3)n2)cc1 ; "M277"] | Theoretical value 619.2; Measured value 619.6 | Theoretical value: C, 79.46; H, 4.07; N, 11.30; Measured value C, 79.52; H, 4.01; N, 11.25; |
| M289 | 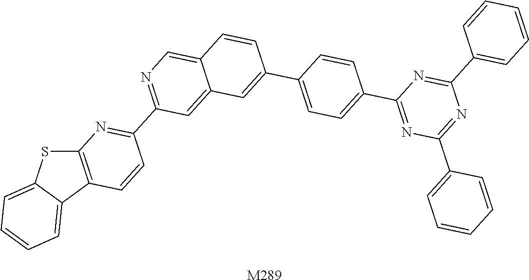 [SMILES: c1ccc(-c2nc(-c3ccccc3)nc(-c3ccc(-c4ccc5cnc(-c6ccc7c(n6)sc6ccccc67)cc5c4)cc3)n2)cc1 ; "M289"] | Theoretical value 619.2; Measured value 619.3 | Theoretical value: C, 79.46; H, 4.07; N, 11.30; Measured value C, 79.54; H, 4.03; N, 11.26; |
| M301 | 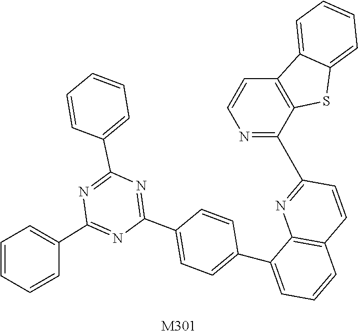 [SMILES: c1ccc(-c2nc(-c3ccccc3)nc(-c3ccc(-c4cccc5ccc(-c6nccc7c6sc6ccccc67)nc45)cc3)n2)cc1 ; "M301"] | Theoretical value 619.2; Measured value 619.2 | Theoretical value: C, 79.46; H, 4.07; N, 11.30; Measured value C, 79.50; H, 4.05; N, 11.24; |
| M314 | 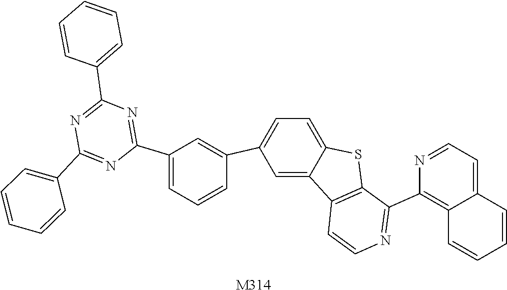 [SMILES: c1ccc(-c2nc(-c3ccccc3)nc(-c3cccc(-c4ccc5sc6c(-c7nccc8ccccc78)nccc6c5c4)c3)n2)cc1 ; "M314"] | Theoretical value 619.2; Measured value 619.5 | Theoretical value: C, 79.46; H, 4.07; N, 11.30; Measured value C, 79.53; H, 4.00; N, 11.27; |
| M325 | 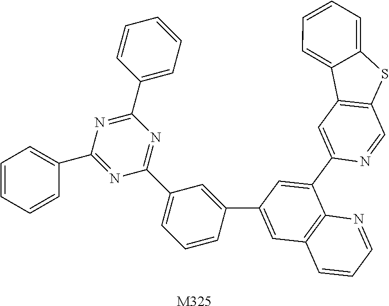 [SMILES: c1ccc(-c2nc(-c3ccccc3)nc(-c3cccc(-c4cc(-c5cc6c(cn5)sc5ccccc56)c5ncccc5c4)c3)n2)cc1 ; "M325"] | Theoretical value 619.2; Measured value 619.6 | Theoretical value: C, 79.46; H, 4.07; N, 11.30; Measured value C, 79.58; H, 3.99; N, 11.23; |
| M337 | 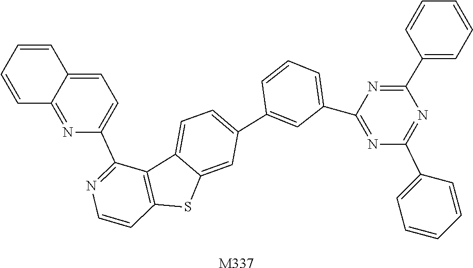 [SMILES: c1ccc(-c2nc(-c3ccccc3)nc(-c3cccc(-c4ccc5c(c4)sc4ccnc(-c6ccc7ccccc7n6)c45)c3)n2)cc1 ; "M337"] | Theoretical value 619.2; Measured value 619.2 | Theoretical value: C, 79.46; H, 4.07; N, 11.30; Measured value C, 79.50; H, 4.01; N, 11.28; |
| M349 | 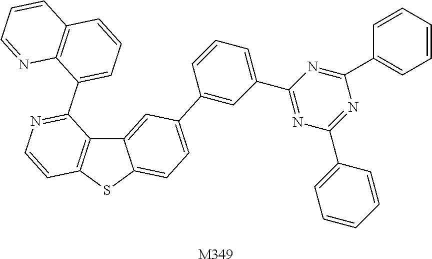 [SMILES: c1ccc(-c2nc(-c3ccccc3)nc(-c3cccc(-c4ccc5sc6ccnc(-c7cccc8cccnc78)c6c5c4)c3)n2)cc1 ; "M349"] | Theoretical value 619.2; Measured value 619.3 | Theoretical value: C, 79.46; H, 4.07; N, 11.30; Measured value C, 79.51; H, 4.03; N, 11.23; |
| M361 | 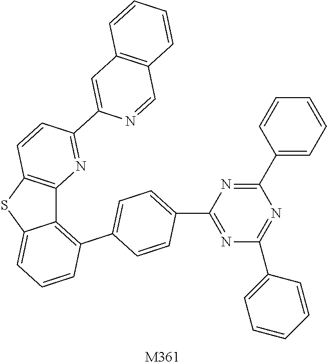 [SMILES: c1ccc(-c2nc(-c3ccccc3)nc(-c3ccc(-c4cccc5sc6ccc(-c7cc8ccccc8cn7)nc6c45)cc3)n2)cc1 ; "M361"] | Theoretical value 619.2; Measured value 619.4 | Theoretical value: C, 79.46; H, 4.07; N, 11.30; Measured value C, 79.53; H, 4.02; N, 11.25; |
| M373 | 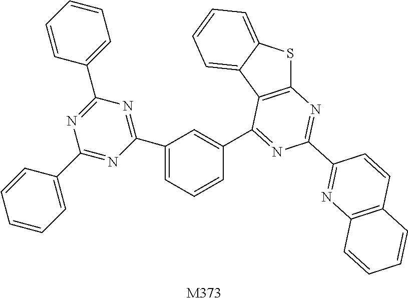 [SMILES: c1ccc(-c2nc(-c3ccccc3)nc(-c3cccc(-c4nc(-c5ccc6ccccc6n5)nc5sc6ccccc6c45)c3)n2)cc1 ; "M373"] | Theoretical value 620.2; Measured value 620.3 | Theoretical value: C, 77.40; H, 3.90; N, 13.54; Measured value C, 77.51; H, 3.83; N, 13.51; |
| M385 | 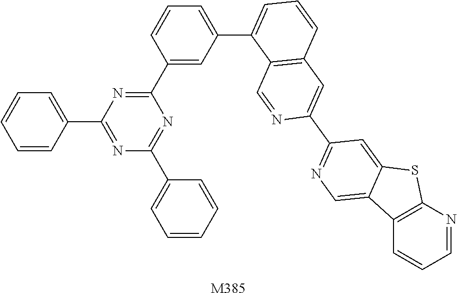 [SMILES: c1ccc(-c2nc(-c3ccccc3)nc(-c3cccc(-c4cccc5cc(-c6cc7sc8ncccc8c7cn6)ncc45)c3)n2)cc1 ; "M385"] | Theoretical value 620.2; Measured value 620.5 | Theoretical value: C, 77.40; H, 3.90; N, 13.54; Measured value C, 77.49; H, 3.85; N, 13.47; |
| M410 | 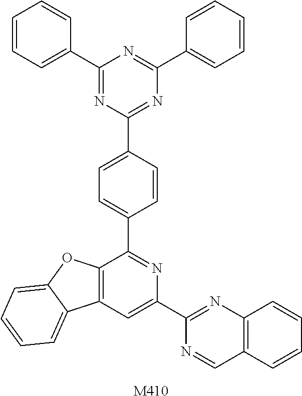 [SMILES: c1ccc(-c2nc(-c3ccccc3)nc(-c3ccc(-c4nc(-c5ncc6ccccc6n5)cc5c4oc4ccccc45)cc3)n2)cc1 ; "M410"] | Theoretical value 604.2; Measured value 604.5 | Theoretical value: C, 79.45; H, 4.00; N, 13.90; Measured value C, 79.54; H, 3.94; N, 13.88; |
| M419 | 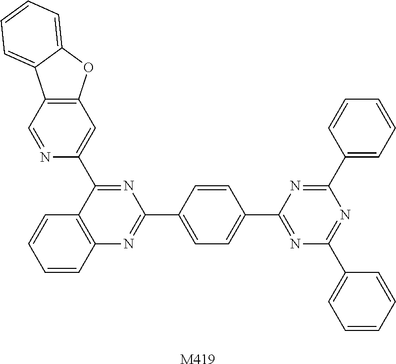 [SMILES: c1ccc(-c2nc(-c3ccccc3)nc(-c3ccc(-c4nc(-c5cc6oc7ccccc7c6cn5)c5ccccc5n4)cc3)n2)cc1 ; "M419"] | Theoretical value 604.2; Measured value 604.3 | Theoretical value: C, 79.45; H, 4.00; N, 13.90; Measured value C, 79.58; H, 3.94; N, 13.85; |
| M445 | 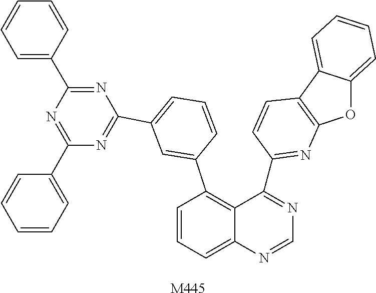 | Theoretical value 604.2; Measured value 604.1 | Theoretical value: C, 79.45; H, 4.00; N, 13.90; Measured value C, 79.56; H, 392; N, 13.89; |
| M458 | 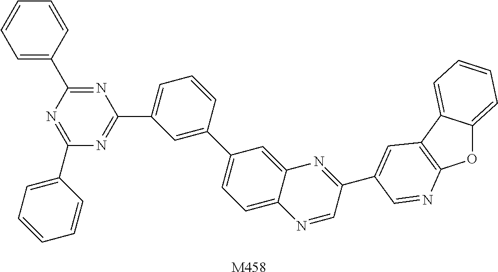 [SMILES: c1ccc(-c2nc(-c3ccccc3)nc(-c3cccc(-c4ccc5ncc(-c6cnc7oc8ccccc8c7c6)nc5c4)c3)n2)cc1 ; "M458"] | Theoretical value 604.2; Measured value 604.2 | Theoretical value: C, 79.45; H, 4.00; N, 13.90; Measured value C, 79.46; H, 3.98; N, 13.88; |
| M475 | 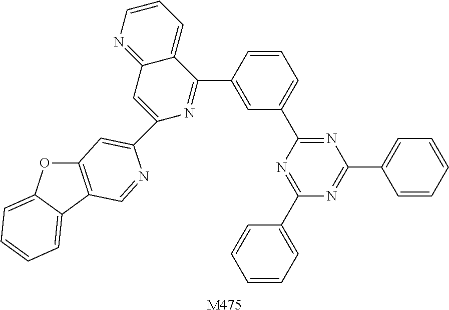 | Theoretical value 604.2; Measured value 604.5 | Theoretical value: C, 79.45; H, 4.00; N, 13.90; Measured value C, 79.53; H, 3.96; N, 13.86; |
| M499 | 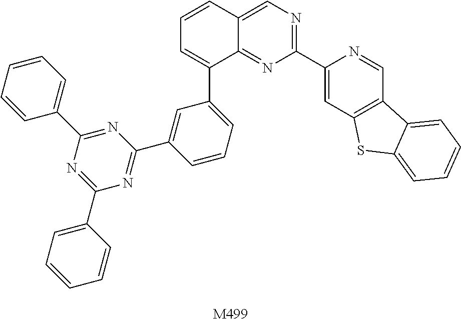 [SMILES: c1ccc(-c2nc(-c3ccccc3)nc(-c3cccc(-c4cccc5cnc(-c6cc7sc8ccccc8c7cn6)nc45)c3)n2)cc1 ; "M499"] | Theoretical value 620.2; Measured value 620.3 | Theoretical value: C, 77.40; H, 3.90; N, 13.54; Measured value C, 77.52; H, 3.85; N, 13.48; |
| M512 | 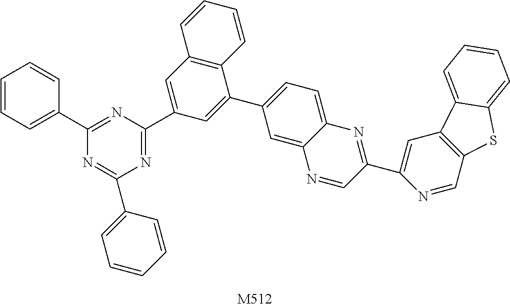 [SMILES: c1ccc(-c2nc(-c3ccccc3)nc(-c3cc(-c4ccc5nc(-c6cc7c(cn6)sc6ccccc67)cnc5c4)c4ccccc4c3)n2)cc1 ; "M512"] | Theoretical value 670.2; Measured value 670.4 | Theoretical value: C, 78.78; H, 3.91; N, 12.53; Measured value C, 78.89; H, 3.87; N, 12.49; |
| M528 | 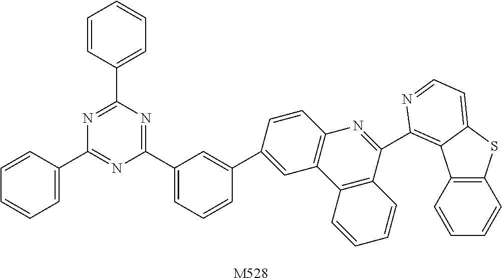 [SMILES: c1ccc(-c2nc(-c3ccccc3)nc(-c3cccc(-c4ccc5nc(-c6nccc7sc8ccccc8c67)c6ccccc6c5c4)c3)n2)cc1 ; "M528"] | Theoretical value 669.2; Measured value 669.6 | Theoretical value: C, 80.69; H, 4.06; N, 10.46; Measured value C, 80.77; H, 4.01; N, 10.42; |
| M535 | 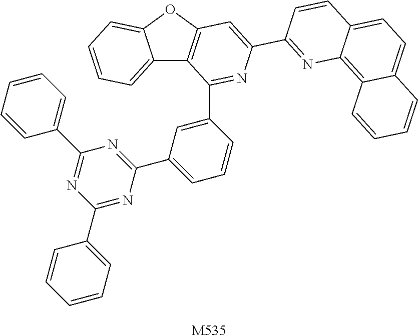 [SMILES: c1ccc(-c2nc(-c3ccccc3)nc(-c3cccc(-c4nc(-c5ccc6ccc7ccccc7c6n5)cc5oc6ccccc6c45)c3)n2)cc1 ; "M535"] | Theoretical value 653.2; Measured value 653.3 | Theoretical value: C, 82.68; H, 4.16; N, 10.71; Measured value C, 82.79; H, 4.09; N, 10.66; |
| M547 | 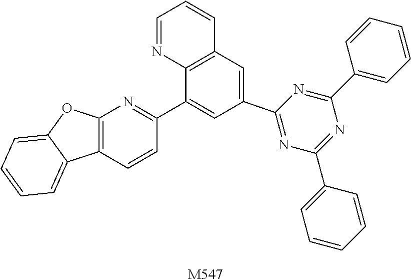 [SMILES: c1ccc(-c2nc(-c3ccccc3)nc(-c3cc(-c4ccc5c(n4)oc4ccccc45)c4ncccc4c3)n2)cc1 ; "M547"] | Theoretical value 527.2; Measured value 527.2 | Theoretical value: C, 79.68; H, 4.01; N, 13.27; Measured value C, 79.76; H, 3.96; N, 13.22; |
| M549 | 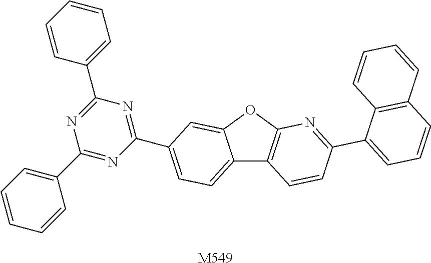 [SMILES: c1ccc(-c2nc(-c3ccccc3)nc(-c3ccc4c(c3)oc3nc(-c5cccc6ccccc56)ccc34)n2)cc1 ; "M549"] | Theoretical value 527.2; Measured value 527.5 | Theoretical value: C, 79.68; H, 4.01; N, 13.27; Measured value C, 79.73; H, 3.98; N, 13.23; |
| M549 | 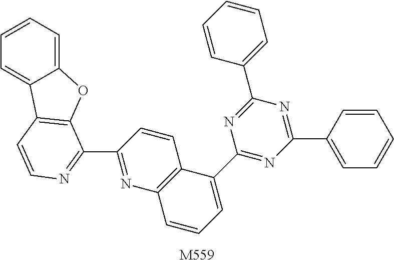 | Theoretical value 527.2; Measured value 527.4 | Theoretical value: C, 79.68; H, 4.01; N, 13.27; Measured value C, 79.78; H, 3.94; N, 13.25; |
| M583 | 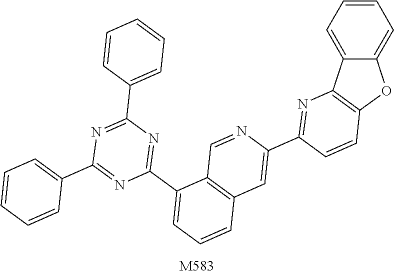 | Theoretical value 527.2; Measured value 527.3 | Theoretical value: C, 79.68; H, 4.01; N, 13.27; Measured value C, 79.71; H, 3.98; N, 13.26; |

Compound Performance Test:

Simulation Calculation of Energy Levels of Compound

Simulation calculations of energy levels of the compounds of each example and comparative example were performed using density functional theory (DFT). The distributions of the molecular frontier orbitals HOMO and LUMO of the compounds of the present application were optimized and calculated using the Gaussian 09 program package (Guassian Inc.) at the B3LYP/6-31G(d) calculation level, as shown in Table 2. In one embodiment, the simulation calculations of the singlet energy level Si and triplet energy level Ti of each compound molecule were performed on the basis of the time-dependent density functional theory (TD-DFT), and the results are shown in Table 2.

TABLE 2

| Compound | LUMO | HOMO | T₁ | S₁ |
|---|---|---|---|---|
| M001 | 1.97 | 5.69 | 2.38 | 3.32 |
| M008 | 1.98 | 5.76 | 2.60 | 3.40 |
| M013 | 2.01 | 5.88 | 2.51 | 3.45 |
| M025 | 1.94 | 5.86 | 2.47 | 3.59 |
| M037 | 2.09 | 5.71 | 2.40 | 3.27 |
| M049 | 2.07 | 6.02 | 2.67 | 3.58 |
| M061 | 2.00 | 5.87 | 2.42 | 3.44 |
| M073 | 1.87 | 5.91 | 2.62 | 3.62 |
| M083 | 2.00 | 5.93 | 2.55 | 3.55 |
| M086 | 2.02 | 5.85 | 2.57 | 3.44 |
| M088 | 1.94 | 5.78 | 2.46 | 3.45 |
| M096 | 1.95 | 5.80 | 2.57 | 3.44 |
| M103 | 1.99 | 5.76 | 2.36 | 3.31 |
| M110 | 1.98 | 5.89 | 2.63 | 3.49 |
| M121 | 1.78 | 5.70 | 2.40 | 3.37 |
| M133 | 1.80 | 5.85 | 2.68 | 3.62 |
| M140 | 1.95 | 5.71 | 2.42 | 3.34 |
| M151 | 1.82 | 5.88 | 2.56 | 3.61 |
| M159 | 2.11 | 5.99 | 2.69 | 3.48 |
| M169 | 1.98 | 5.65 | 2.52 | 3.23 |
| M181 | 1.90 | 5.85 | 2.68 | 3.56 |
| M193 | 2.01 | 5.89 | 2.43 | 3.39 |
| M205 | 2.07 | 5.90 | 2.43 | 3.46 |
| M217 | 1.94 | 5.81 | 2.41 | 3.43 |
| M229 | 1.91 | 5.94 | 2.58 | 3.57 |
| M243 | 2.05 | 6.14 | 2.59 | 3.59 |
| M253 | 1.85 | 5.88 | 2.49 | 3.63 |
| M277 | 1.94 | 5.82 | 2.45 | 3.50 |
| M289 | 2.07 | 5.78 | 2.38 | 3.35 |
| M301 | 1.91 | 5.81 | 2.46 | 3.52 |
| M314 | 1.93 | 5.67 | 2.41 | 3.24 |
| M325 | 1.86 | 5.64 | 2.43 | 3.43 |
| M337 | 1.88 | 5.87 | 2.66 | 3.54 |
| M349 | 1.83 | 5.78 | 2.73 | 3.53 |
| M361 | 1.92 | 5.59 | 2.52 | 2.84 |
| M373 | 2.05 | 6.07 | 2.56 | 3.51 |
| M385 | 1.91 | 5.91 | 2.53 | 3.60 |
| M410 | 1.98 | 5.85 | 2.54 | 3.48 |
| M419 | 2.14 | 6.06 | 2.48 | 3.44 |
| M445 | 2.04 | 5.93 | 2.51 | 3.41 |
| M458 | 2.05 | 6.16 | 2.56 | 3.35 |
| M475 | 1.95 | 5.95 | 2.54 | 3.61 |
| M499 | 2.05 | 5.94 | 2.47 | 3.34 |
| M512 | 2.09 | 5.80 | 2.39 | 3.29 |
| M528 | 1.89 | 5.86 | 2.61 | 3.57 |
| M535 | 1.80 | 5.68 | 2.57 | 3.50 |
| M547 | 2.08 | 5.90 | 2.52 | 3.39 |
| M549 | 2.03 | 5.94 | 2.63 | 3.52 |
| M559 | 2.02 | 6.07 | 2.48 | 3.66 |
| M583 | 2.10 | 5.66 | 2.34 | 3.15 |

From the data in Table 2, it can be seen that the organic compounds provided by the present disclosure all have Si values higher than 2.8 eV, no absorption in the visible light region, Ti higher than 2.4 eV, and LUMO values between 1.7 and 2.2 eV, which are thus suitable for use as electron transport material.

The application effects of the compounds synthesized in the present disclosure as electron transport layer material in a device will be described in detail below through the Device Examples 1-50 and Device Comparative Examples 1-2. The Device Examples 2-50 and the Device Comparative Examples 1-2 have exactly the same fabrication process of the device as the Device Example 1, but differ from the Device Example 1 in that the material of the electron transport layer of the device was changed.

### Device Example 1

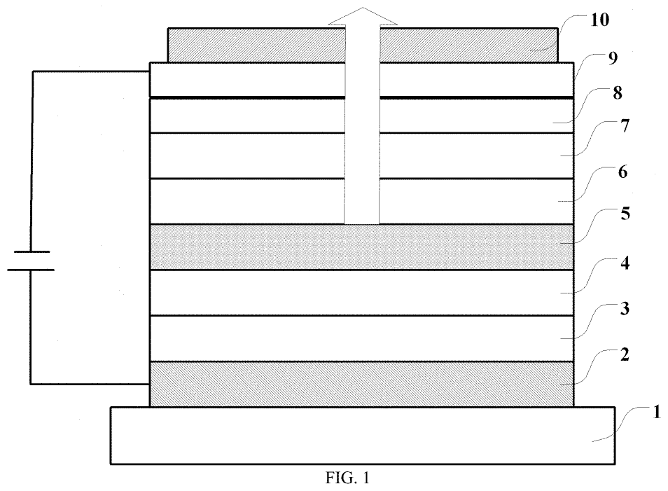

This application example provides an OLED device, as shown in FIG. 1. FIG. 1 is the schematic structural diagram of the organic light-emitting device provided by the present disclosure, comprising a glass substrate plate 1, an anode 2, a hole injection layer 3, a first hole transport layer 4, a second hole transport layer 5, a light emitting layer 6, a hole blocking layer 7, an electron transport layer 8, a cathode 9 and a cap layer 10.

The Preparation Steps of the OLED Device are as Follows:

(1) The glass substrate plate 1 was cut into a size of 50 mm×50 mm×0.7 mm, sonicated in isopropanol and deionized water for 30 min, respectively, and then exposed to ozone for about 10 min for cleaning. The resulting glass substrate plate 1 with an indium tin oxide (ITO) anode was mounted on a vacuum deposition apparatus;

(2) Compound 1 and compound 2 were vacuum deposited on the ITO anode layer 2 as the hole injection layer 3, and the doping ratio of compound 1 was 2% (mass ratio), and the thickness of the hole injection layer 3 was 10 nm;

(3) The hole transport layer material compound 2 was vacuum deposited on the hole injection layer 3 at a thickness of 120 nm as the first hole transport layer 4;

(4) The hole transport material compound 3 was vacuum deposited on the first hole transport layer 4 at a thickness of 5 nm as the second hole transport layer 5;

(5) The light-emitting layer 6 was vacuum deposited on the second hole transport layer 5, and compound 4 was used as the host material, compound 5 was used as the doping material at a doping ratio of 3% (mass ratio), and the thickness of the light-emitting layer 6 was 30 nm;

(6) Compound 6 was vacuum deposited on the light-emitting layer 6 at a thickness of 5 nm as the hole blocking layer 7;

(7) Compound 7 and compound M001 were vacuum deposited on the hole blocking layer 7 as the electron transport layer 8, and the doping ratio of compound 7 and compound M001 was 1:1, and the thickness of the electron transport layer 8 was 30 nm;

(8) Magnesium and silver were vacuum deposited on the electron transport layer 8 as the cathode 9, and Mg:Ag was 1:9, and the thickness of the cathode 9 was 15 nm;

(9) Compound 8 was vacuum deposited on the cathode 9 at a thickness of 70 nm as the cap layer 10.

The structures of the compounds used in the OLED device are as follows:

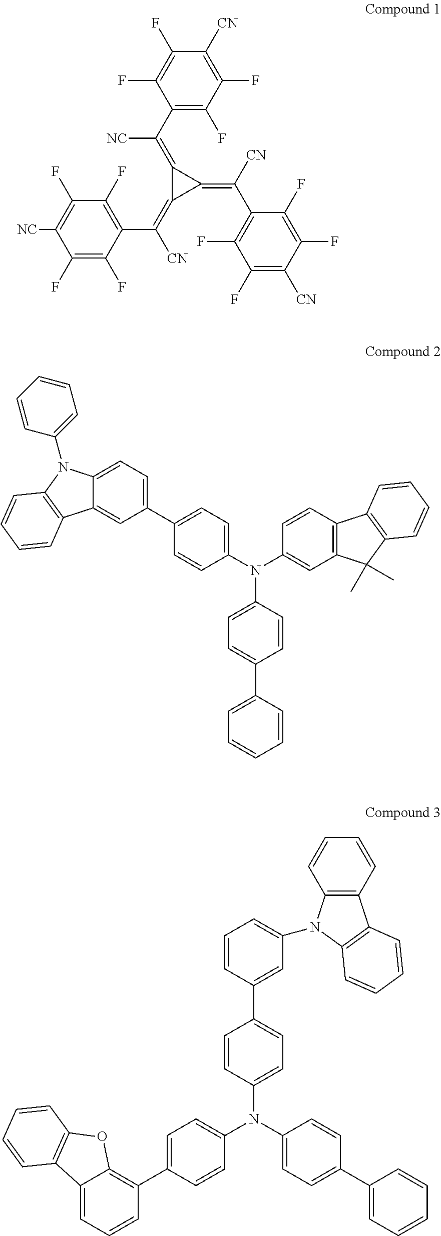

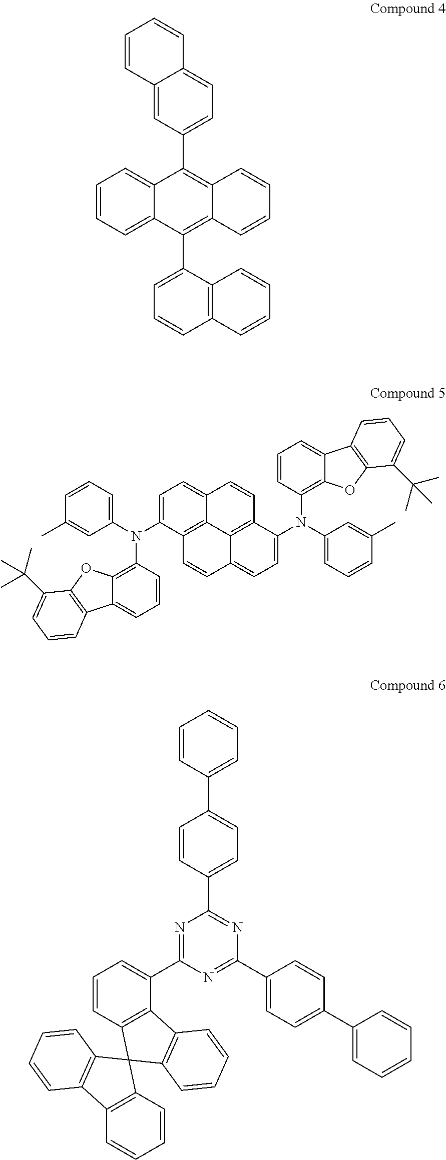

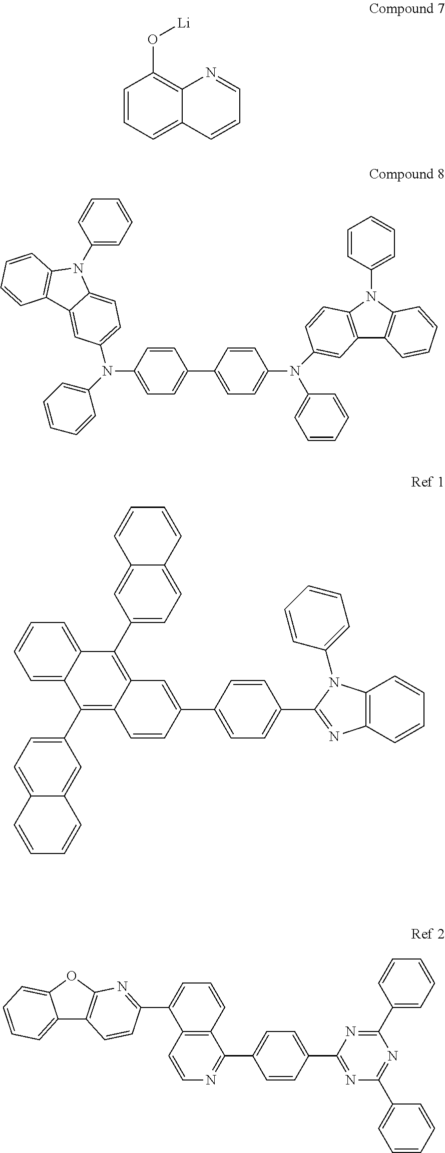

### Device Example 2 to Device Example 50

The organic electroluminescent devices were prepared by using the same method as in Device Example 1, except that the compounds shown in Table 2 were used to replace the electron transport compound M001.

### Device Comparative Example 1

This comparative example only differs from the application example 1 in that the organic compound M001 in step (7) was replaced with an equivalent amount of the comparative compound Ref1, while other preparation steps were the same.

### Device Comparative Example 2

This comparative example only differs from the application example 1 in that the organic compound M001 in step (7) was replaced with an equivalent amount of the comparative compound Ref2, while other preparation steps were the same.

Performance evaluation of OLED devices:

The current of the OLED device at different voltages was tested using the Keithley 2365A digital nanovoltmeter, and then the current was divided by the light-emitting area to obtain the current density of the OLED device at different voltages. The brightness and radiant energy flux density of the OLED device under different voltages were tested using the Konicaminolta CS-2000 spectroradiometer. According to the current density and brightness of the OLED device under different voltages, the working voltage V and current efficiency (cd/A) under the same current density (10 mA/cm²) were obtained. The time when the brightness of the OLED device reached 95% of the initial brightness was measured to obtain the lifetime T95 (under the test condition of 20 mA/cm²). The performance data are shown in Table 3.

After the fabrication of the electroluminescent device was completed according to the above steps, the driving voltage, efficiency and lifetime of the device were measured. The results are shown in Table 3.

Table 3 Test data of devices in Device Examples 1-50 and Device Comparative

### Examples 1-2

| | Electron | Driving | Current | LT95 |
|---|---|---|---|---|
| | transport | voltage | efficiency | (20 mA/ |
| Number | layer | (10 mA/cm²) | (10 mA/cm²) | cm²) |
| Device Example | M001 | 95% | 115% | 126% |
| 1 | | | | |
| Device Example | M008 | 95% | 113% | 128% |
| 2 | | | | |
| Device Example | M013 | 96% | 113% | 123% |
| 3 | | | | |
| Device Example | M025 | 95% | 114% | 132% |
| 4 | | | | |
| Device Example | M037 | 98% | 112% | 127% |
| 5 | | | | |
| Device Example | M049 | 98% | 113% | 120% |
| 6 | | | | |
| Device Example | M061 | 95% | 115% | 135% |
| 7 | | | | |
| Device Example | M073 | 95% | 118% | 133% |
| 8 | | | | |
| Device Example | M083 | 96% | 114% | 129% |
| 9 | | | | |
| Device Example | M086 | 96% | 115% | 130% |
| 10 | | | | |
| Device Example | M088 | 95% | 117% | 132% |
| 11 | | | | |
| Device Example | M096 | 95% | 116% | 128% |
| 12 | | | | |
| Device Example | M103 | 96% | 115% | 125% |
| 13 | | | | |
| Device Example | M110 | 96% | 115% | 130% |
| 14 | | | | |
| Device Example | M121 | 98% | 112% | 123% |
| 15 | | | | |
| Device Example | M133 | 98% | 113% | 120% |
| 16 | | | | |
| Device Example | M140 | 95% | 115% | 130% |
| 17 | | | | |
| Device Example | M151 | 97% | 114% | 126% |
| 18 | | | | |
| Device Example | M159 | 98% | 111% | 119% |
| 19 | | | | |
| Device Example | M169 | 96% | 115% | 129% |
| 20 | | | | |
| Device Example | M181 | 96% | 115% | 132% |
| 21 | | | | |
| Device Example | M193 | 95% | 118% | 131% |
| 22 | | | | |
| Device Example | M205 | 98% | 113% | 124% |
| 23 | | | | |
| Device Example | M217 | 95% | 116% | 129% |
| 24 | | | | |
| Device Example | M229 | 95% | 116% | 126% |
| 25 | | | | |
| Device Example | M243 | 96% | 115% | 131% |
| 26 | | | | |
| Device Example | M253 | 97% | 113% | 122% |
| 27 | | | | |
| Device Example | M277 | 95% | 117% | 129% |
| 28 | | | | |
| Device Example | M289 | 98% | 112% | 121% |
| 29 | | | | |
| Device Example | M301 | 95% | 118% | 134% |
| 30 | | | | |
| Device Example | M314 | 96% | 114% | 116% |
| 31 | | | | |
| Device Example | M325 | 97% | 113% | 123% |
| 32 | | | | |
| Device Example | M337 | 97% | 115% | 118% |
| 33 | | | | |
| Device Example | M349 | 97% | 113% | 121% |
| 34 | | | | |
| Device Example | M361 | 96% | 116% | 127% |
| 35 | | | | |
| Device Example | M373 | 96% | 117% | 128% |
| 36 | | | | |
| Device Example | M385 | 96% | 116% | 119% |
| 37 | | | | |
| Device Example | M410 | 96% | 115% | 128% |
| 38 | | | | |
| Device Example | M419 | 99% | 110% | 110% |
| 39 | | | | |
| Device Example | M445 | 96% | 115% | 124% |
| 40 | | | | |
| Device Example | M458 | 96% | 116% | 129% |
| 41 | | | | |
| Device Example | M475 | 95% | 118% | 130% |
| 42 | | | | |
| Device Example | M499 | 96% | 116% | 132% |
| 43 | | | | |
| Device Example | M512 | 97% | 113% | 126% |
| 44 | | | | |
| Device Example | M528 | 96% | 117% | 121% |
| 45 | | | | |
| Device Example | M535 | 98% | 112% | 126% |
| 46 | | | | |
| Device Example | M547 | 98% | 112% | 117% |
| 47 | | | | |
| Device Example | M549 | 96% | 116% | 124% |
| 48 | | | | |
| Device Example | M559 | 96% | 117% | 129% |
| 49 | | | | |
| Device Example | M583 | 98% | 111% | 119% |
| 50 | | | | |
| Device | Ref1 | 100% | 100% | 100% |
| Compareative | | | | |
| Example 1 | | | | |
| Device | Ref2 | 99.5% | 101% | 102% |
| Compareative | | | | |
| Example 2 | | | | |

From the data in Table 3, it can be seen that the OLED devices prepared based on the organic compounds provided by the Examples 1 to 50 of the present disclosure as electron transport layer materials have lower working voltage, higher current efficiency and longer working lifetime as compared with those prepared based on the comparative compounds Ref1 and Ref2. It can be seen that the chemical structure of the compound provided by the present disclosure can be closely combined with Liq (8-hydroxyquinolinolato-lithium), which effectively alleviates the unfavorable situation that the metal lithium is moved by the heat and electric field generated during the driving process of the device, and effectively prolongs the lifetime of the OLED device.

What is claimed is:

1. A heterocyclic compound having the structure as shown in formula I: 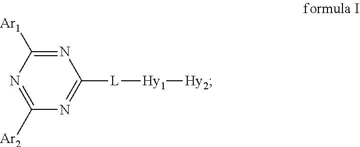
wherein, Ar₁ and Ar₂ are independently selected from the group consisting of hydrogen, deuterium, substituted or unsubstituted C₆-C₁₈ aryl or C₂-C₁₈ heteroaryl;
L is selected from the group consisting of single bond, substituted or unsubstituted C₆-C₃₀ arylene;
Hy₁ and Hy₂ are independently selected from the structures represented by formula II or formula III, and Hy₁ and Hy₂ are different; 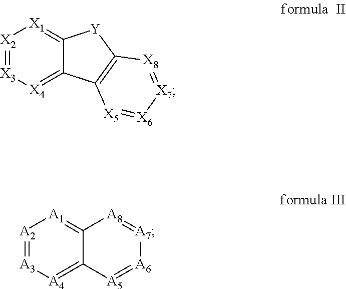
Y is O or S;
X₁, X₂, X₃, X₄, X₅, X₆, X₇, X₈, A₁, A₂, A₃, A₄, A₅, A₆, A₇ and A₈ are independently selected from the group consisting of N atom or CR₁; at least one of X₁, X₂, X₃, X₄, X₅, X₆, X₇, and X₈ is an N atom, and at least one of A₁, A₂, A₃, A₄, A₅, A₆, A₇, and A₈ is an N atom;
R₁ is selected from the group consisting of hydrogen, deuterium, substituted or unsubstituted C₁-C₆ alkyl or C₂-C₆ alkenyl, and adjacent R₁ may be connected to form a ring.

2. The organic compound having a nitrogen-containing heterocyclic structure according to claim 1, wherein the substituent of the C₆-C₁₈ aryl or the C₂-C₁₈ heteroaryl is one or more selected from the group consisting of deuterium, halogen, cyano, methyl, ethyl, isopropyl, tert-butyl, adamantyl, and triphenylsilyl;
the substituent of the C₆-C₃₀ arylene is one or more selected from the group consisting of deuterium, halogen, cyano, methyl, ethyl, isopropyl, tert-butyl, adamantyl, and triphenylsilyl;
the substituent of the C₁-C₆ alkyl or the C₂-C₆ alkenyl is one or more selected from the group consisting of deuterium, halogen, cyano, methyl, ethyl, isopropyl, tert-butyl, adamantyl, and triphenylsilane.

3. The organic compound having a nitrogen-containing heterocyclic structure according to claim 1, wherein the Ar₁ and Ar₂ are independently selected from the group consisting of hydrogen, deuterium, phenyl, biphenyl, naphthyl, anthracenyl, phenanthryl, thienyl, furanyl, benzothienyl, benzofuranyl, dibenzofuranyl, dibenzothienyl or pyridyl.

4. The organic compound having a nitrogen-containing heterocyclic structure according to claim 1, wherein L is selected from the group consisting of single bond, phenylene, naphthylene, phenanthrylene, anthracenylene, pyrrolylidene, thienylene, furanylene, benzofuranylene, benzothienylene, dibenzofuranylene, dibenzothienylene, pyridinylene, pyrimidinylene, pyridazinylene, triazinylene, pyrazinylene, quinolinylene, quinoxalinylene or isoquinolinylene.

5. The organic compound having a nitrogen-containing heterocyclic structure according to claim 1, wherein the formula II has any one of the following structures:

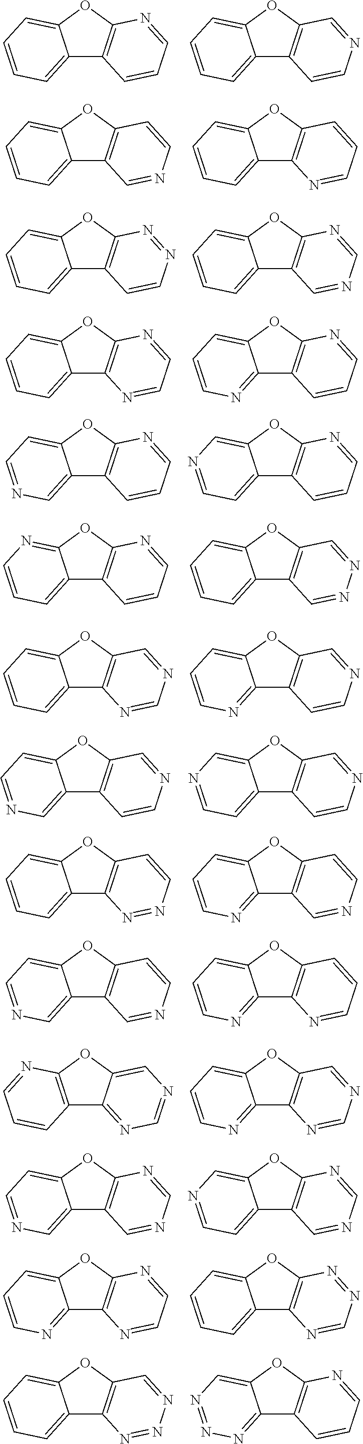

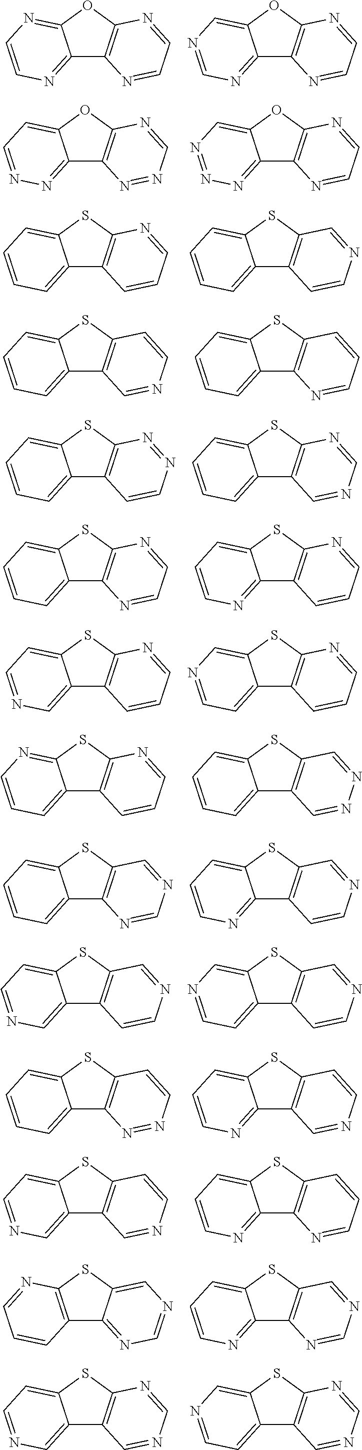

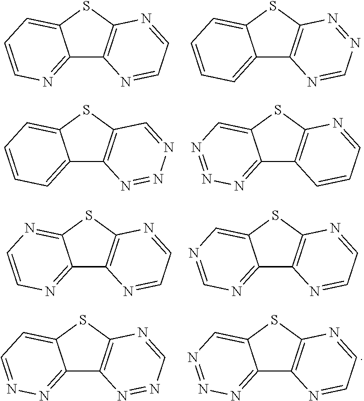

6. The organic compound having a nitrogen-containing heterocyclic structure according to claim 1, wherein the formula III has any one of the following structures:

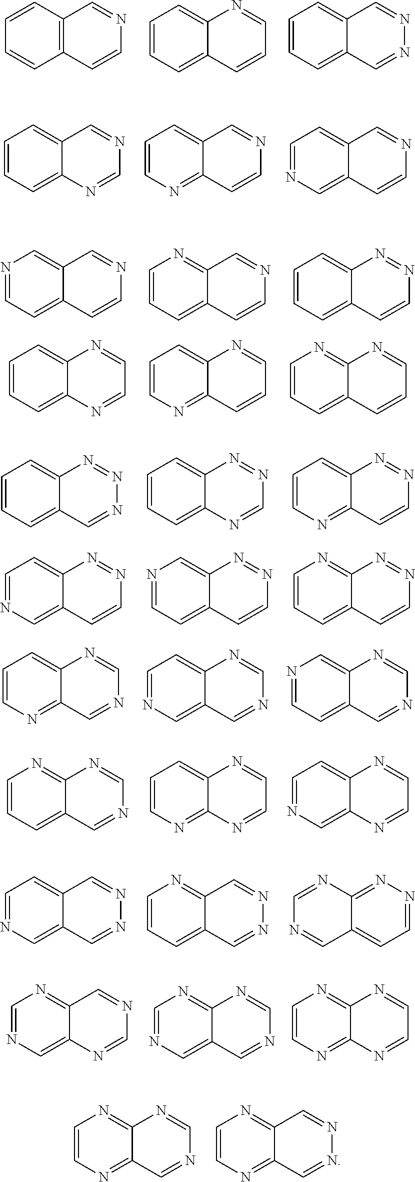

7. The organic compound having a nitrogen-containing heterocyclic structure according to claim 1, wherein the formula III has any one of the following structures:

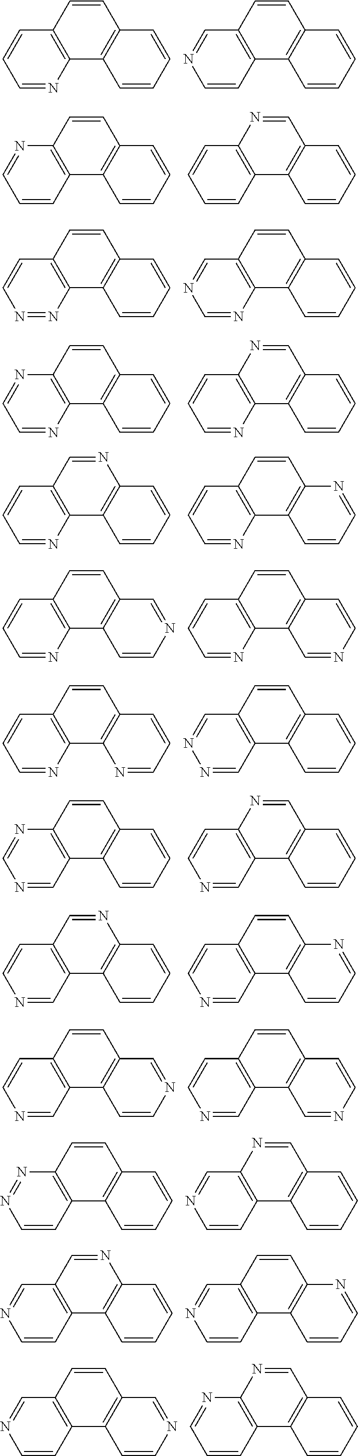

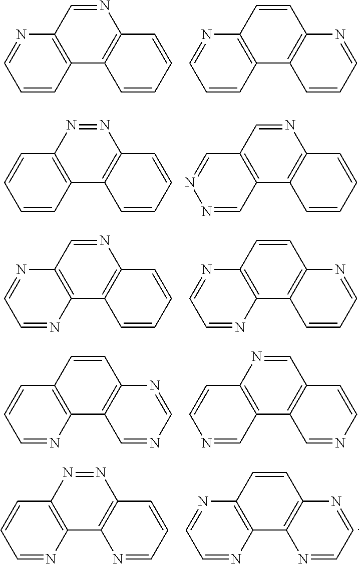

8. The organic compound having a nitrogen-containing heterocyclic structure according to claim 1, wherein the Hy₁ and Hy₂ are connected through any C atom in the Hy₁ structure and any C atom in the Hy₂ structure, wherein one side or both sides adjacent to the connecting C atom in the Hy₁ is an N atom, or one side or both sides adjacent to the connecting C atom in the Hy₂ is an N atom.

9. The organic compound having a nitrogen-containing heterocyclic structure according to claim 1, wherein the Hy₁ and Hy₂ are connected through any C atom in the Hy₁ structure and any C atom in the Hy₂ structure, wherein one side or both sides adjacent to the connecting C atom in the Hy₁ is an N atom, and one side or both sides adjacent to the connecting C atom in the Hy₂ is an N atom.

10. The organic compound having a nitrogen-containing heterocyclic structure according to claim 1, wherein the Hy₁ and Hy₂ are connected through the C atom connected with #in the Hy₁ structure and any C atom in the Hy₂ structure, wherein one side or both sides adjacent to the connecting C atom in the Hy₂ is an N atom, and the Hy₁ is selected from the following structure: 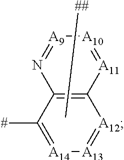
A₉, A₁₀, A₁₁, A₁₂, A₁₃, and A₁₄ are independently selected from the group consisting of N atom or CR₂;
R₂ is selected from the group consisting of hydrogen, deuterium, substituted or unsubstituted C₁-C₆ alkyl or C₂-C₆ alkenyl, and adjacent R₂ can be connected to form a ring;
the Hy₂ has any one of the following structures: 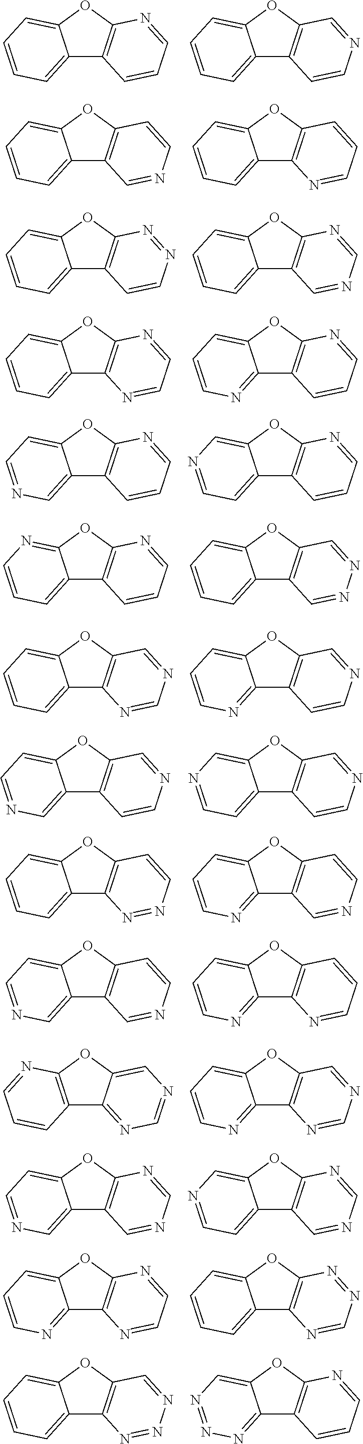 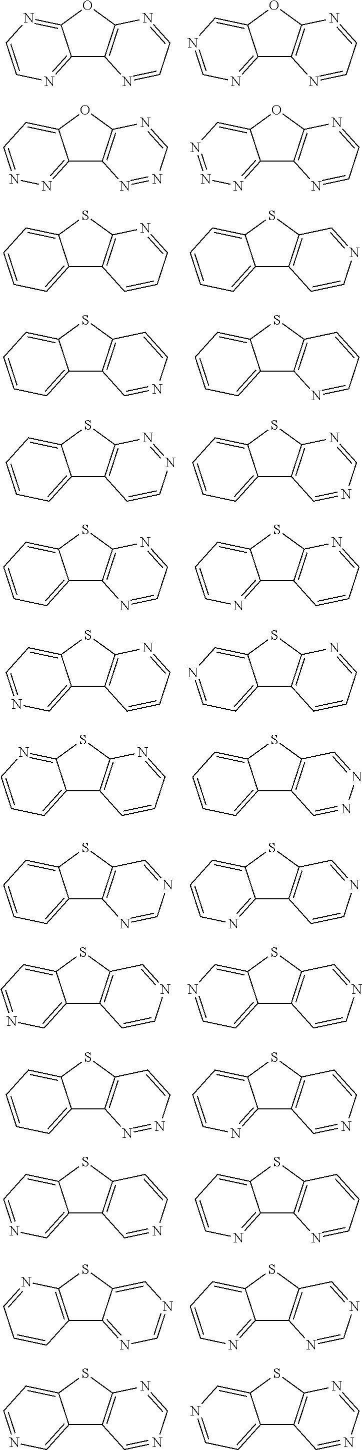 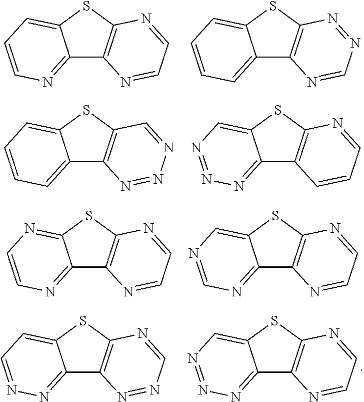
#represents the connecting position of Hy₁ and Hy₁;
##represents the connecting position of Hy₁ and L.

11. The organic compound having a nitrogen-containing heterocyclic structure according to claim 1, wherein the Hy₁ and Hy₂ are connected through any C atom in the Hy₁ structure and the C atom connected with #in the Hy₂ structure, wherein one side or both sides adjacent to the connecting C atom in the Hy₁ is an N atom, and the Hy₂ is selected from the following structure: 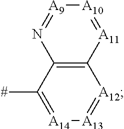
A₉, A₁₀, A₁₁, A₁₂, A₁₃, and A₁₄ are independently selected from the group consisting of N atom or CR₂;
R₂ is selected from the group consisting of hydrogen, deuterium, substituted or unsubstituted C₁-C₆ alkyl or C₂-C₆ alkenyl, and adjacent R₂ can be connected to form a ring;
the Hy₁ has any one of the following structures: 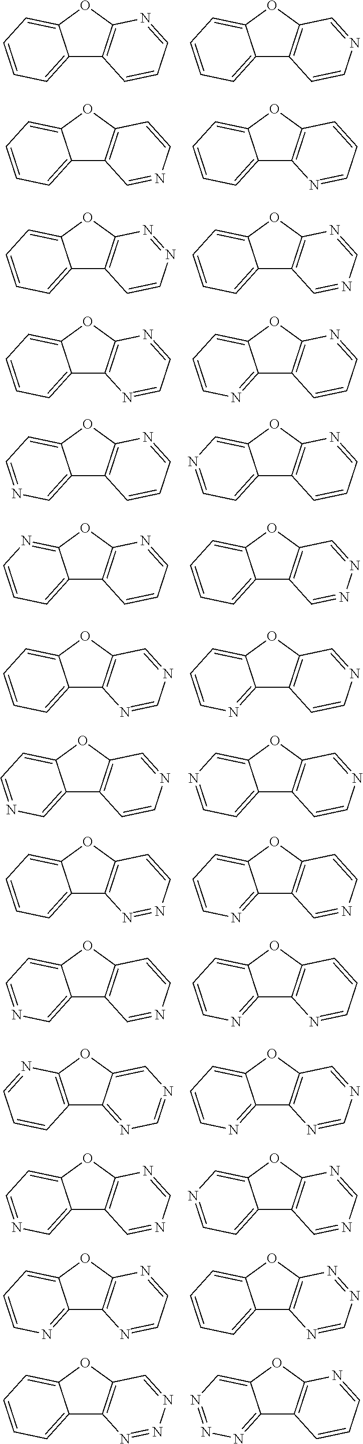 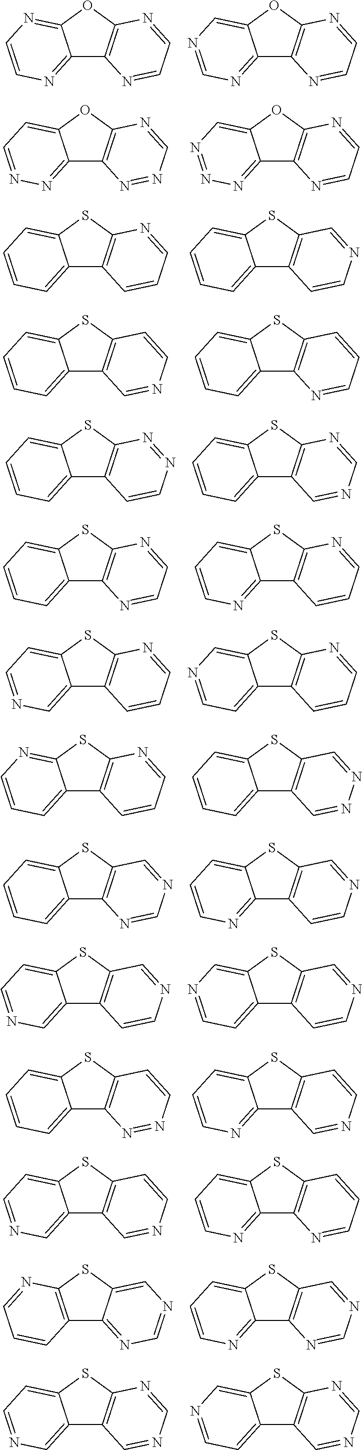 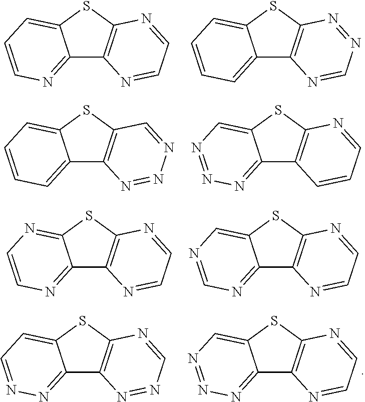
#represents the connecting position of Hy₁ and Hy₁.

12. The organic compound having a nitrogen-containing heterocyclic structure according to claim 1, wherein it has any one of the following structures:

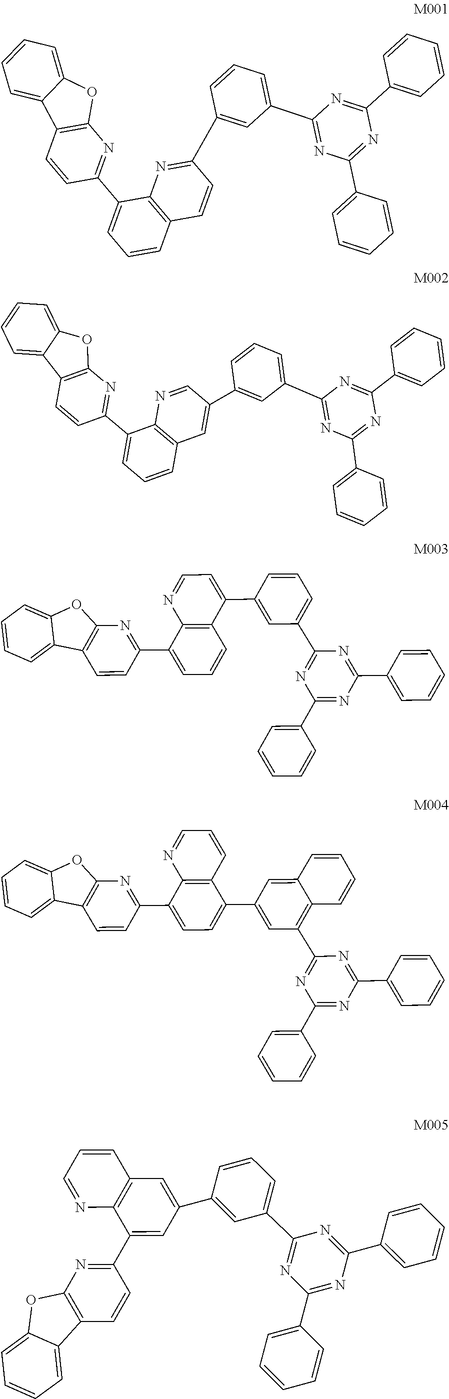 [SMILES: c1ccc(-c2nc(-c3ccccc3)nc(-c3cccc(-c4ccc5cccc(-c6ccc7c(n6)oc6ccccc67)c5n4)c3)n2)cc1 ; c1ccc(-c2nc(-c3ccccc3)nc(-c3cccc(-c4cnc5c(-c6ccc7c(n6)oc6ccccc67)cccc5c4)c3)n2)cc1 ; c1ccc(-c2nc(-c3ccccc3)nc(-c3cccc(-c4ccnc5c(-c6ccc7c(n6)oc6ccccc67)cccc45)c3)n2)cc1 ; c1ccc(-c2nc(-c3ccccc3)nc(-c3cc(-c4ccc(-c5ccc6c(n5)oc5ccccc56)c5ncccc45)cc4ccccc34)n2)cc1 ; c1ccc(-c2nc(-c3ccccc3)nc(-c3cccc(-c4cc(-c5ccc6c(n5)oc5ccccc56)c5ncccc5c4)c3)n2)cc1]

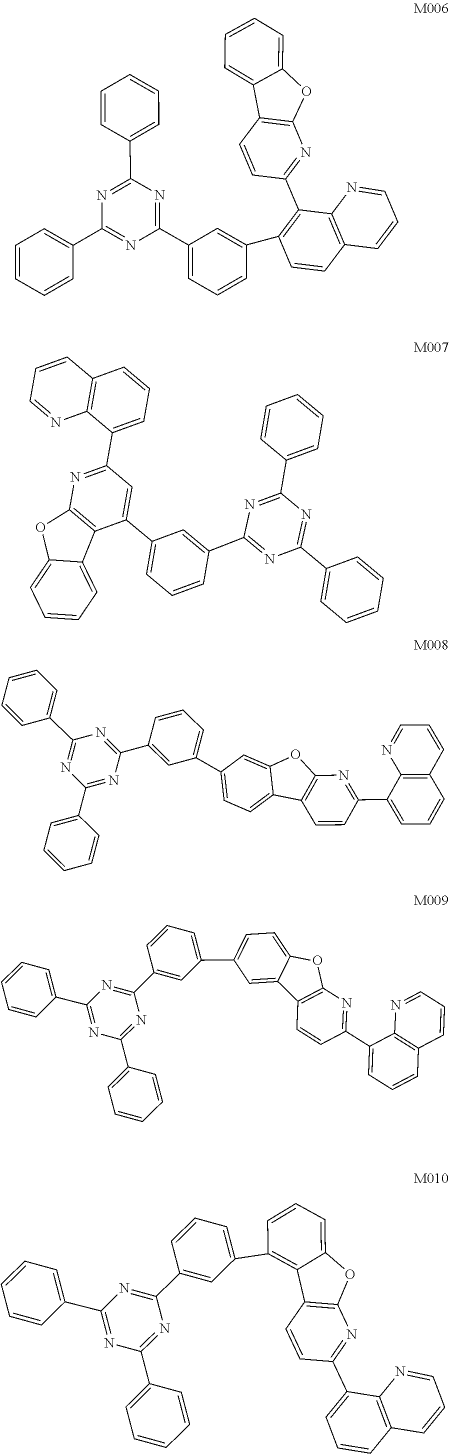 [SMILES: c1ccc(-c2nc(-c3ccccc3)nc(-c3cccc(-c4ccc5cccnc5c4-c4ccc5c(n4)oc4ccccc45)c3)n2)cc1 ; c1ccc(-c2nc(-c3ccccc3)nc(-c3cccc(-c4cc(-c5cccc6cccnc56)nc5oc6ccccc6c45)c3)n2)cc1 ; c1ccc(-c2nc(-c3ccccc3)nc(-c3cccc(-c4ccc5c(c4)oc4nc(-c6cccc7cccnc67)ccc45)c3)n2)cc1 ; c1ccc(-c2nc(-c3ccccc3)nc(-c3cccc(-c4ccc5oc6nc(-c7cccc8cccnc78)ccc6c5c4)c3)n2)cc1 ; c1ccc(-c2nc(-c3ccccc3)nc(-c3cccc(-c4cccc5oc6nc(-c7cccc8cccnc78)ccc6c45)c3)n2)cc1]

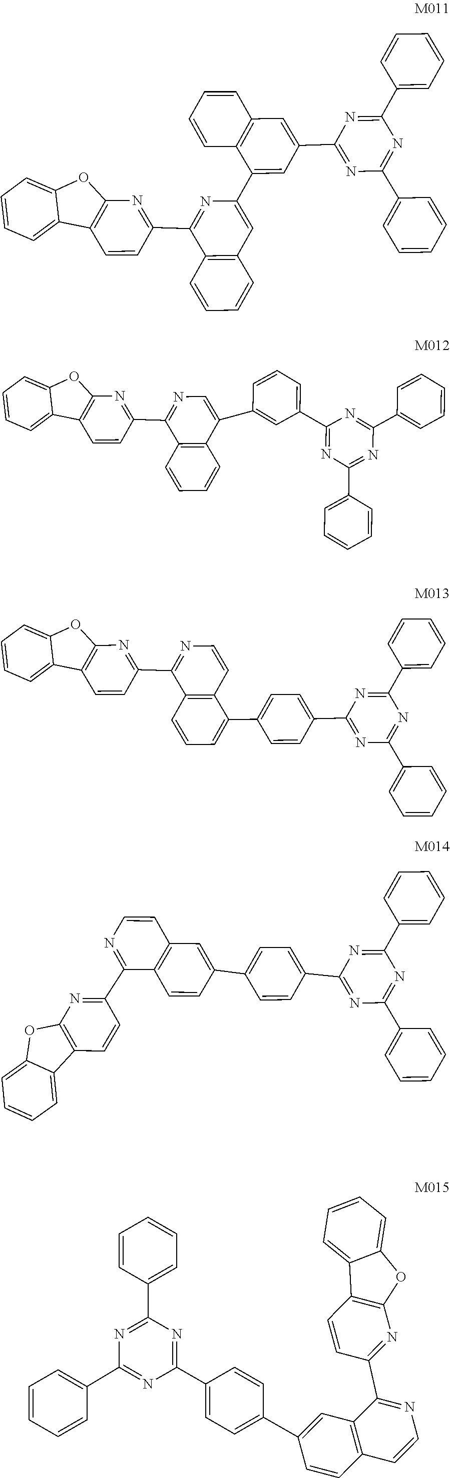 [SMILES: c1ccc(-c2nc(-c3ccccc3)nc(-c3cc(-c4cc5ccccc5c(-c5ccc6c(n5)oc5ccccc56)n4)c4ccccc4c3)n2)cc1 ; c1ccc(-c2nc(-c3ccccc3)nc(-c3cccc(-c4cnc(-c5ccc6c(n5)oc5ccccc56)c5ccccc45)c3)n2)cc1 ; c1ccc(-c2nc(-c3ccccc3)nc(-c3ccc(-c4cccc5c(-c6ccc7c(n6)oc6ccccc67)nccc45)cc3)n2)cc1 ; c1ccc(-c2nc(-c3ccccc3)nc(-c3ccc(-c4ccc5c(-c6ccc7c(n6)oc6ccccc67)nccc5c4)cc3)n2)cc1 ; c1ccc(-c2nc(-c3ccccc3)nc(-c3ccc(-c4ccc5ccnc(-c6ccc7c(n6)oc6ccccc67)c5c4)cc3)n2)cc1]

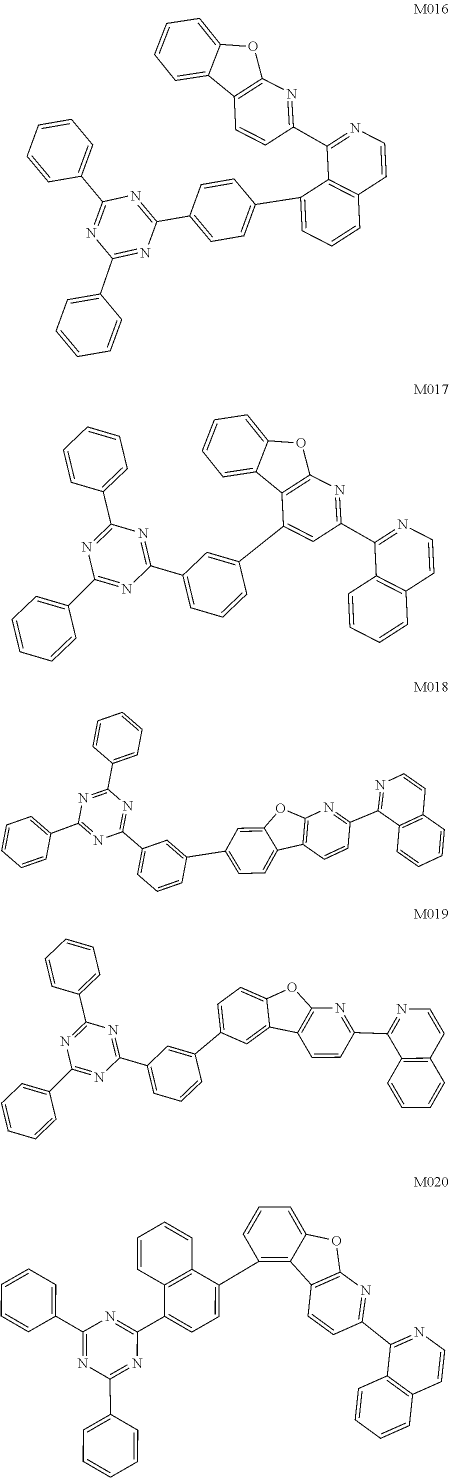 [SMILES: c1ccc(-c2nc(-c3ccccc3)nc(-c3ccc(-c4cccc5ccnc(-c6ccc7c(n6)oc6ccccc67)c45)cc3)n2)cc1 ; c1ccc(-c2nc(-c3ccccc3)nc(-c3cccc(-c4cc(-c5nccc6ccccc56)nc5oc6ccccc6c45)c3)n2)cc1 ; c1ccc(-c2nc(-c3ccccc3)nc(-c3cccc(-c4ccc5c(c4)oc4nc(-c6nccc7ccccc67)ccc45)c3)n2)cc1 ; c1ccc(-c2nc(-c3ccccc3)nc(-c3cccc(-c4ccc5oc6nc(-c7nccc8ccccc78)ccc6c5c4)c3)n2)cc1 ; c1ccc(-c2nc(-c3ccccc3)nc(-c3ccc(-c4cccc5oc6nc(-c7nccc8ccccc78)ccc6c45)c4ccccc34)n2)cc1]

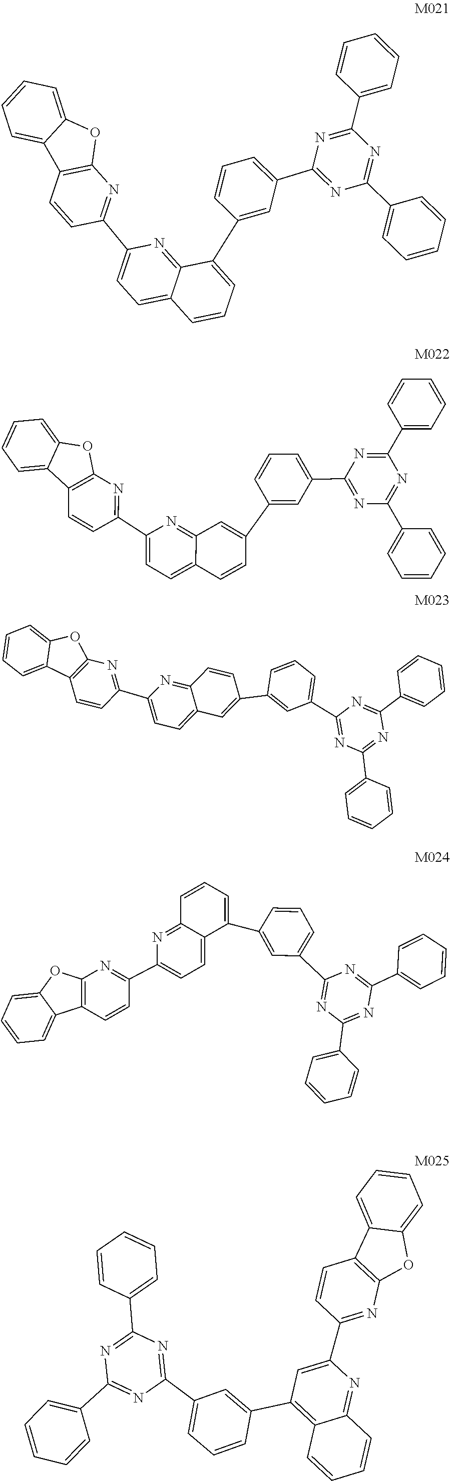 [SMILES: c1ccc(-c2nc(-c3ccccc3)nc(-c3cccc(-c4cccc5ccc(-c6ccc7c(n6)oc6ccccc67)nc45)c3)n2)cc1 ; c1ccc(-c2nc(-c3ccccc3)nc(-c3cccc(-c4ccc5ccc(-c6ccc7c(n6)oc6ccccc67)nc5c4)c3)n2)cc1 ; c1ccc(-c2nc(-c3ccccc3)nc(-c3cccc(-c4ccc5nc(-c6ccc7c(n6)oc6ccccc67)ccc5c4)c3)n2)cc1 ; c1ccc(-c2nc(-c3ccccc3)nc(-c3cccc(-c4cccc5nc(-c6ccc7c(n6)oc6ccccc67)ccc45)c3)n2)cc1 ; c1ccc(-c2nc(-c3ccccc3)nc(-c3cccc(-c4cc(-c5ccc6c(n5)oc5ccccc56)nc5ccccc45)c3)n2)cc1]

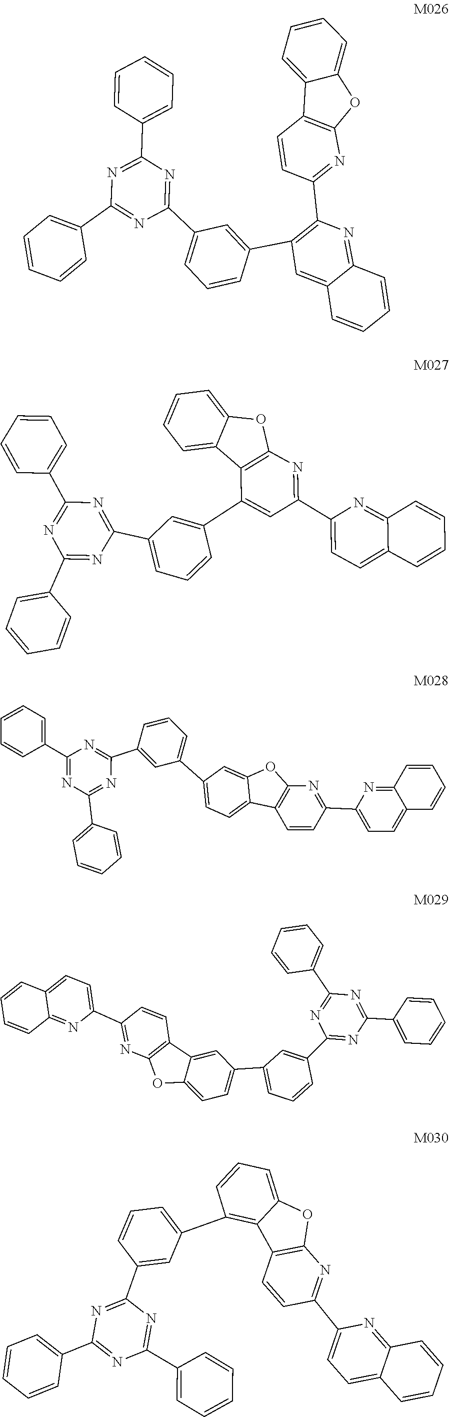 [SMILES: c1ccc(-c2nc(-c3ccccc3)nc(-c3cccc(-c4cc5ccccc5nc4-c4ccc5c(n4)oc4ccccc45)c3)n2)cc1 ; c1ccc(-c2nc(-c3ccccc3)nc(-c3cccc(-c4cc(-c5ccc6ccccc6n5)nc5oc6ccccc6c45)c3)n2)cc1 ; c1ccc(-c2nc(-c3ccccc3)nc(-c3cccc(-c4ccc5c(c4)oc4nc(-c6ccc7ccccc7n6)ccc45)c3)n2)cc1 ; c1ccc(-c2nc(-c3ccccc3)nc(-c3cccc(-c4ccc5oc6nc(-c7ccc8ccccc8n7)ccc6c5c4)c3)n2)cc1 ; c1ccc(-c2nc(-c3ccccc3)nc(-c3cccc(-c4cccc5oc6nc(-c7ccc8ccccc8n7)ccc6c45)c3)n2)cc1]

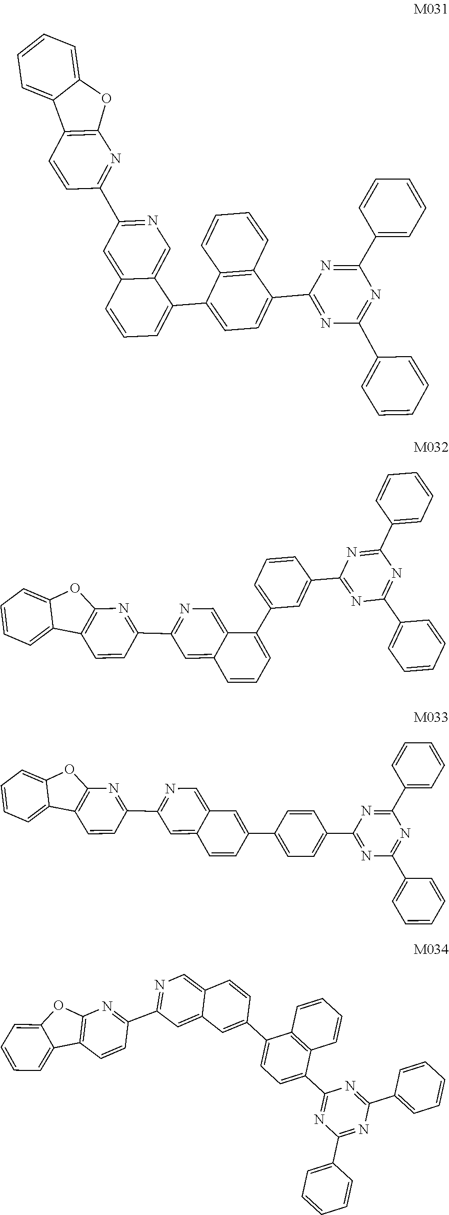 [SMILES: c1ccc(-c2nc(-c3ccccc3)nc(-c3ccc(-c4cccc5cc(-c6ccc7c(n6)oc6ccccc67)ncc45)c4ccccc34)n2)cc1 ; c1ccc(-c2nc(-c3ccccc3)nc(-c3cccc(-c4cccc5cc(-c6ccc7c(n6)oc6ccccc67)ncc45)c3)n2)cc1 ; c1ccc(-c2nc(-c3ccccc3)nc(-c3ccc(-c4ccc5cc(-c6ccc7c(n6)oc6ccccc67)ncc5c4)cc3)n2)cc1 ; c1ccc(-c2nc(-c3ccccc3)nc(-c3ccc(-c4ccc5cnc(-c6ccc7c(n6)oc6ccccc67)cc5c4)c4ccccc34)n2)cc1]

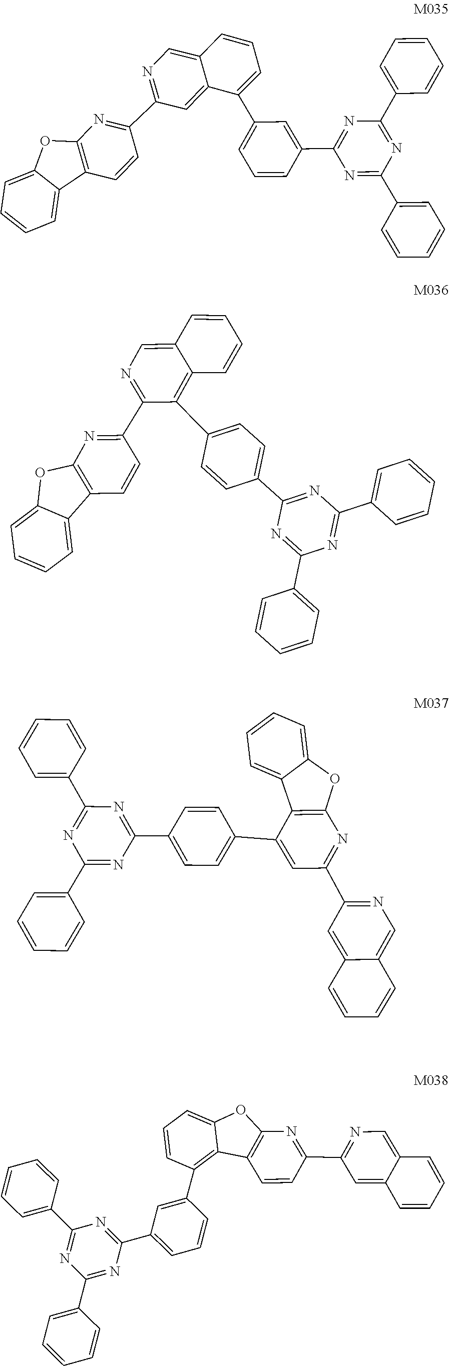 [SMILES: c1ccc(-c2nc(-c3ccccc3)nc(-c3cccc(-c4cccc5cnc(-c6ccc7c(n6)oc6ccccc67)cc45)c3)n2)cc1 ; c1ccc(-c2nc(-c3ccccc3)nc(-c3ccc(-c4c(-c5ccc6c(n5)oc5ccccc56)ncc5ccccc45)cc3)n2)cc1 ; c1ccc(-c2nc(-c3ccccc3)nc(-c3ccc(-c4cc(-c5cc6ccccc6cn5)nc5oc6ccccc6c45)cc3)n2)cc1 ; c1ccc(-c2nc(-c3ccccc3)nc(-c3cccc(-c4cccc5oc6nc(-c7cc8ccccc8cn7)ccc6c45)c3)n2)cc1]

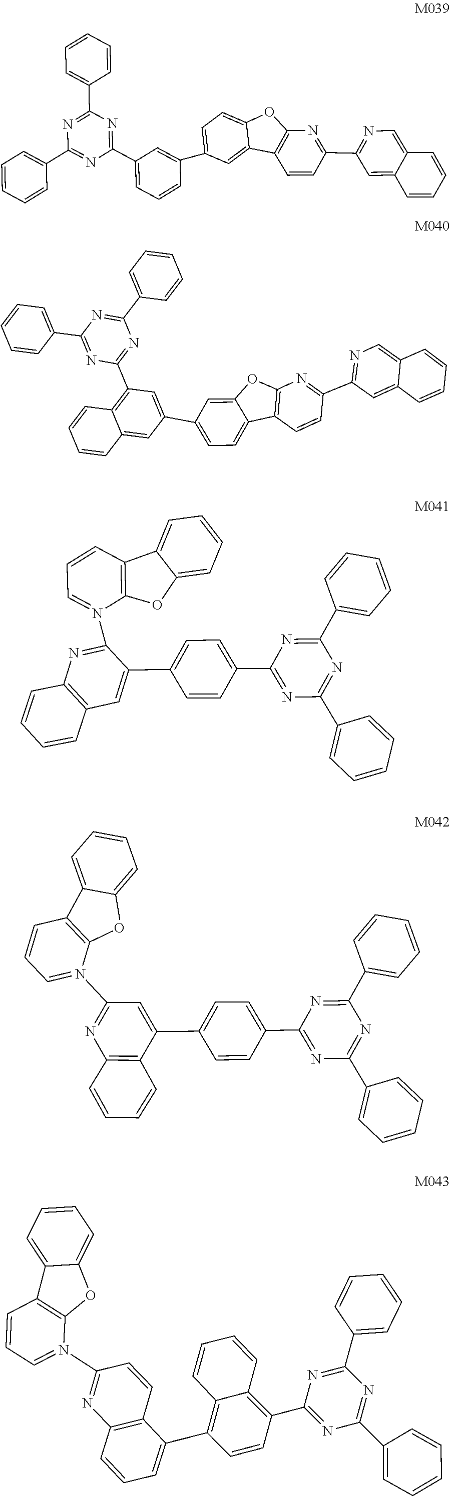

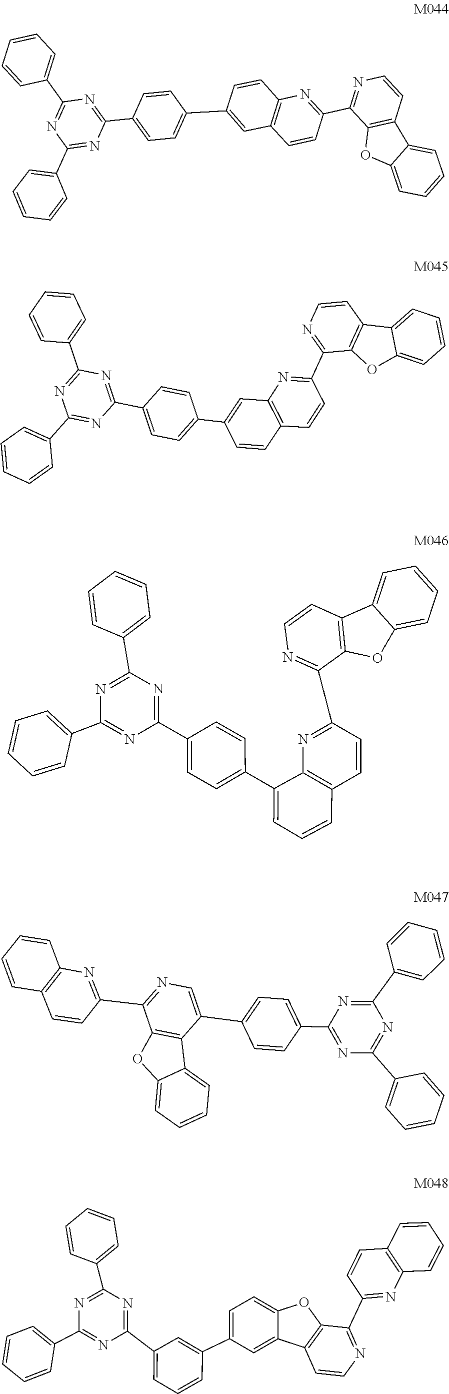 [SMILES: c1ccc(-c2nc(-c3ccccc3)nc(-c3ccc(-c4ccc5nc(-c6nccc7c6oc6ccccc67)ccc5c4)cc3)n2)cc1 ; c1ccc(-c2nc(-c3ccccc3)nc(-c3ccc(-c4ccc5ccc(-c6nccc7c6oc6ccccc67)nc5c4)cc3)n2)cc1 ; c1ccc(-c2nc(-c3ccccc3)nc(-c3ccc(-c4cccc5ccc(-c6nccc7c6oc6ccccc67)nc45)cc3)n2)cc1 ; c1ccc(-c2nc(-c3ccccc3)nc(-c3ccc(-c4cnc(-c5ccc6ccccc6n5)c5oc6ccccc6c45)cc3)n2)cc1 ; c1ccc(-c2nc(-c3ccccc3)nc(-c3cccc(-c4ccc5oc6c(-c7ccc8ccccc8n7)nccc6c5c4)c3)n2)cc1]

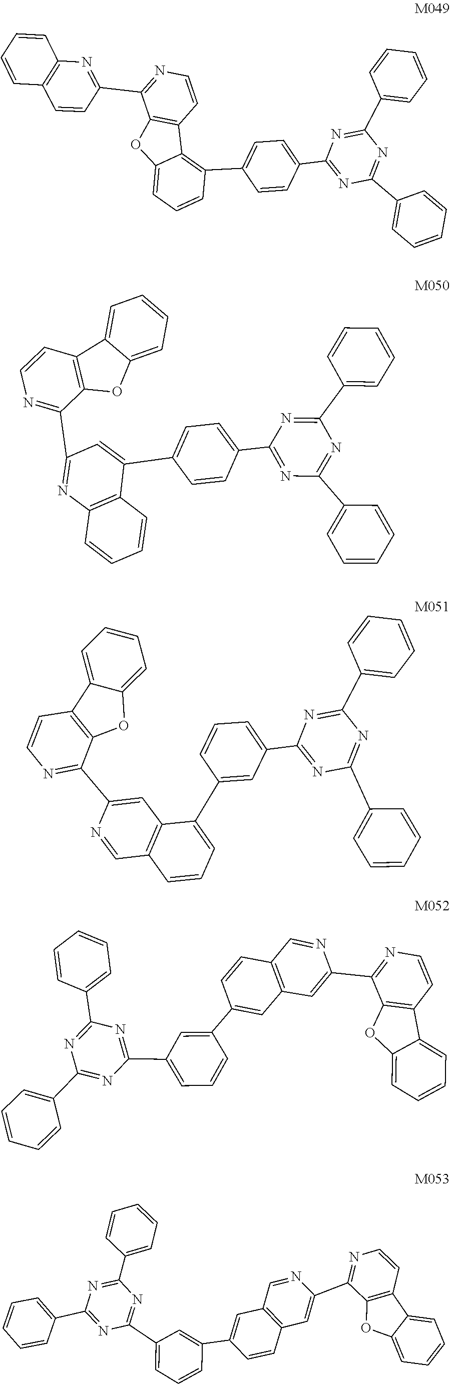 [SMILES: c1ccc(-c2nc(-c3ccccc3)nc(-c3ccc(-c4cccc5oc6c(-c7ccc8ccccc8n7)nccc6c45)cc3)n2)cc1 ; c1ccc(-c2nc(-c3ccccc3)nc(-c3ccc(-c4cc(-c5nccc6c5oc5ccccc56)nc5ccccc45)cc3)n2)cc1 ; c1ccc(-c2nc(-c3ccccc3)nc(-c3cccc(-c4cccc5cnc(-c6nccc7c6oc6ccccc67)cc45)c3)n2)cc1 ; c1ccc(-c2nc(-c3ccccc3)nc(-c3cccc(-c4ccc5cnc(-c6nccc7c6oc6ccccc67)cc5c4)c3)n2)cc1 ; c1ccc(-c2nc(-c3ccccc3)nc(-c3cccc(-c4ccc5cc(-c6nccc7c6oc6ccccc67)ncc5c4)c3)n2)cc1]

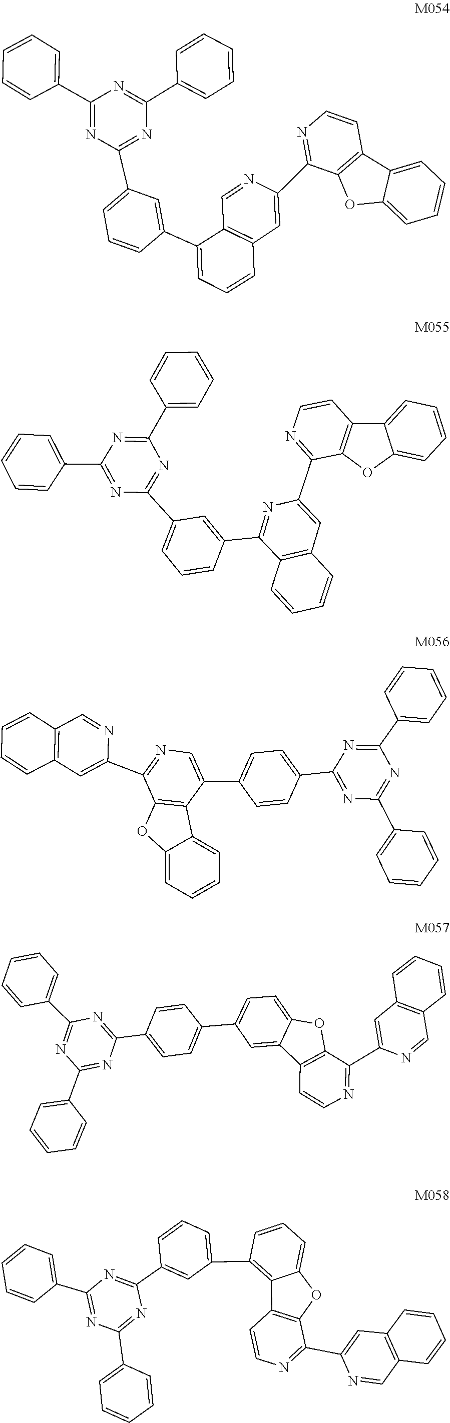 [SMILES: c1ccc(-c2nc(-c3ccccc3)nc(-c3cccc(-c4cccc5cc(-c6nccc7c6oc6ccccc67)ncc45)c3)n2)cc1 ; c1ccc(-c2nc(-c3ccccc3)nc(-c3cccc(-c4nc(-c5nccc6c5oc5ccccc56)cc5ccccc45)c3)n2)cc1 ; c1ccc(-c2nc(-c3ccccc3)nc(-c3ccc(-c4cnc(-c5cc6ccccc6cn5)c5oc6ccccc6c45)cc3)n2)cc1 ; c1ccc(-c2nc(-c3ccccc3)nc(-c3ccc(-c4ccc5oc6c(-c7cc8ccccc8cn7)nccc6c5c4)cc3)n2)cc1 ; c1ccc(-c2nc(-c3ccccc3)nc(-c3cccc(-c4cccc5oc6c(-c7cc8ccccc8cn7)nccc6c45)c3)n2)cc1]

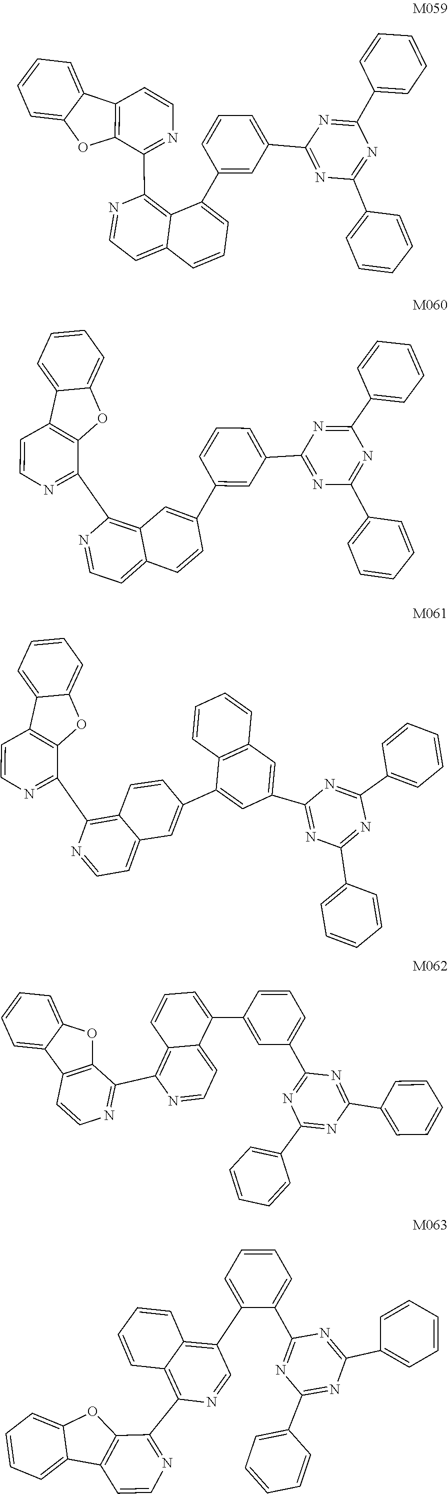

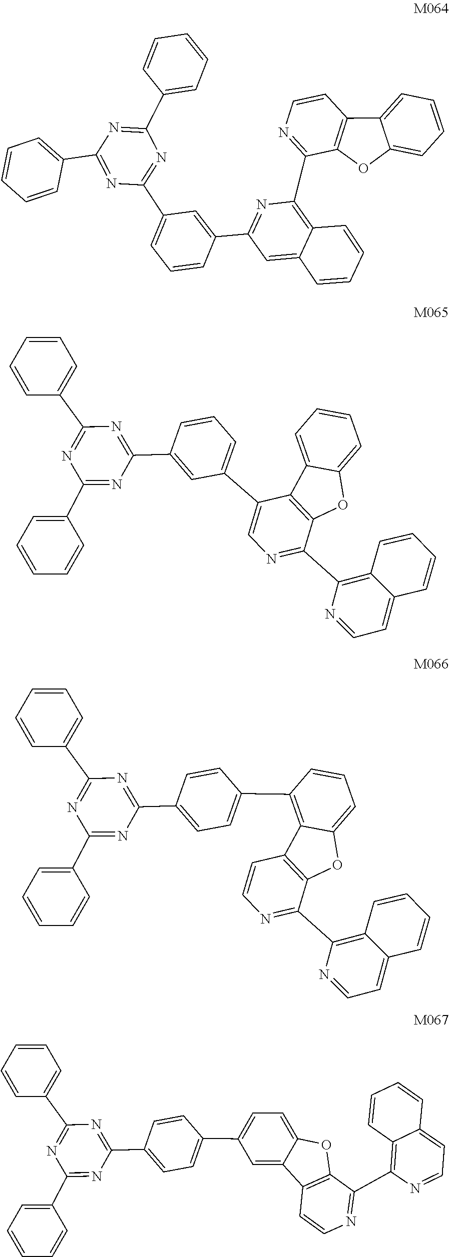 [SMILES: c1ccc(-c2nc(-c3ccccc3)nc(-c3cccc(-c4cc5ccccc5c(-c5nccc6c5oc5ccccc56)n4)c3)n2)cc1 ; c1ccc(-c2nc(-c3ccccc3)nc(-c3cccc(-c4cnc(-c5nccc6ccccc56)c5oc6ccccc6c45)c3)n2)cc1 ; c1ccc(-c2nc(-c3ccccc3)nc(-c3ccc(-c4cccc5oc6c(-c7nccc8ccccc78)nccc6c45)cc3)n2)cc1 ; c1ccc(-c2nc(-c3ccccc3)nc(-c3ccc(-c4ccc5oc6c(-c7nccc8ccccc78)nccc6c5c4)cc3)n2)cc1]

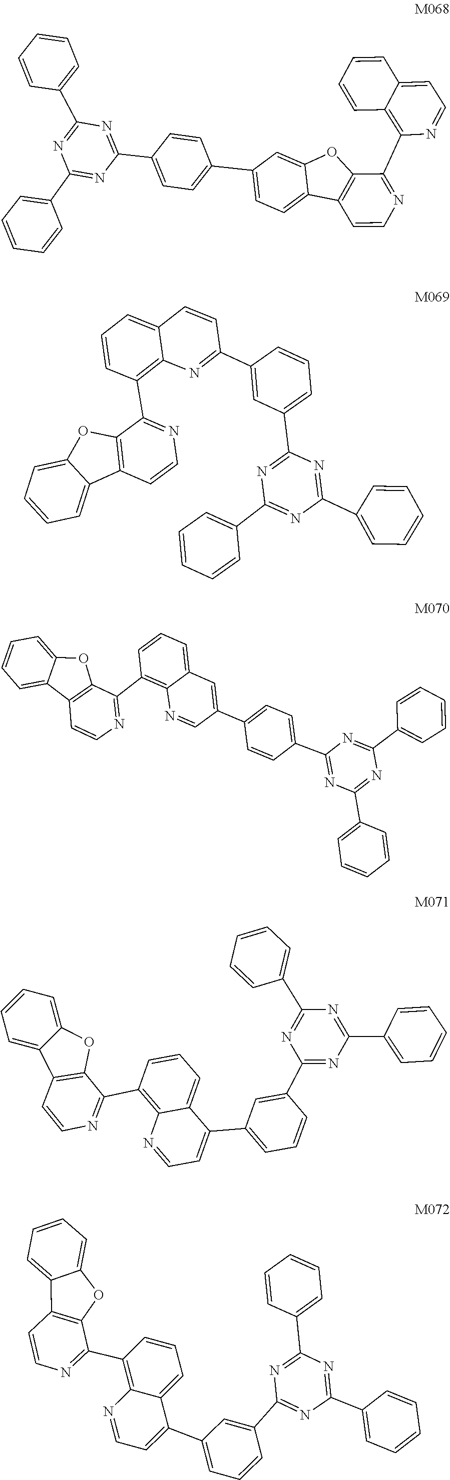 [SMILES: c1ccc(-c2nc(-c3ccccc3)nc(-c3ccc(-c4ccc5c(c4)oc4c(-c6nccc7ccccc67)nccc45)cc3)n2)cc1 ; c1ccc(-c2nc(-c3ccccc3)nc(-c3cccc(-c4ccc5cccc(-c6nccc7c6oc6ccccc67)c5n4)c3)n2)cc1 ; c1ccc(-c2nc(-c3ccccc3)nc(-c3ccc(-c4cnc5c(-c6nccc7c6oc6ccccc67)cccc5c4)cc3)n2)cc1 ; c1ccc(-c2nc(-c3ccccc3)nc(-c3cccc(-c4ccnc5c(-c6nccc7c6oc6ccccc67)cccc45)c3)n2)cc1 ; c1ccc(-c2nc(-c3ccccc3)nc(-c3cccc(-c4ccnc5c(-c6nccc7c6oc6ccccc67)cccc45)c3)n2)cc1]

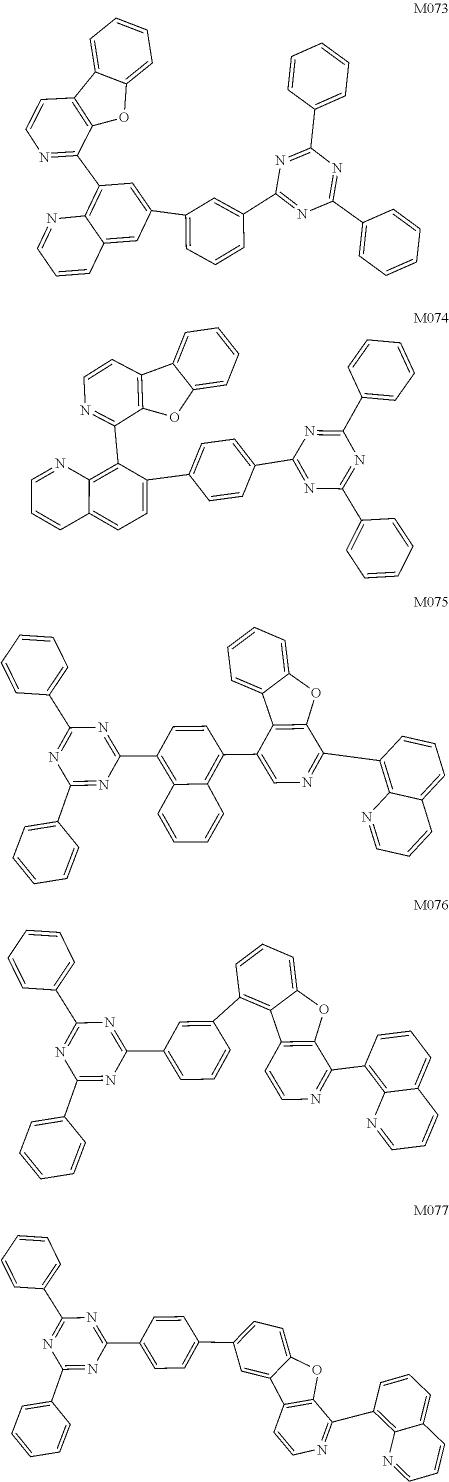 [SMILES: c1ccc(-c2nc(-c3ccccc3)nc(-c3cccc(-c4cc(-c5nccc6c5oc5ccccc56)c5ncccc5c4)c3)n2)cc1 ; c1ccc(-c2nc(-c3ccccc3)nc(-c3ccc(-c4ccc5cccnc5c4-c4nccc5c4oc4ccccc45)cc3)n2)cc1 ; c1ccc(-c2nc(-c3ccccc3)nc(-c3ccc(-c4cnc(-c5cccc6cccnc56)c5oc6ccccc6c45)c4ccccc34)n2)cc1 ; c1ccc(-c2nc(-c3ccccc3)nc(-c3cccc(-c4cccc5oc6c(-c7cccc8cccnc78)nccc6c45)c3)n2)cc1 ; c1ccc(-c2nc(-c3ccccc3)nc(-c3ccc(-c4ccc5oc6c(-c7cccc8cccnc78)nccc6c5c4)cc3)n2)cc1]

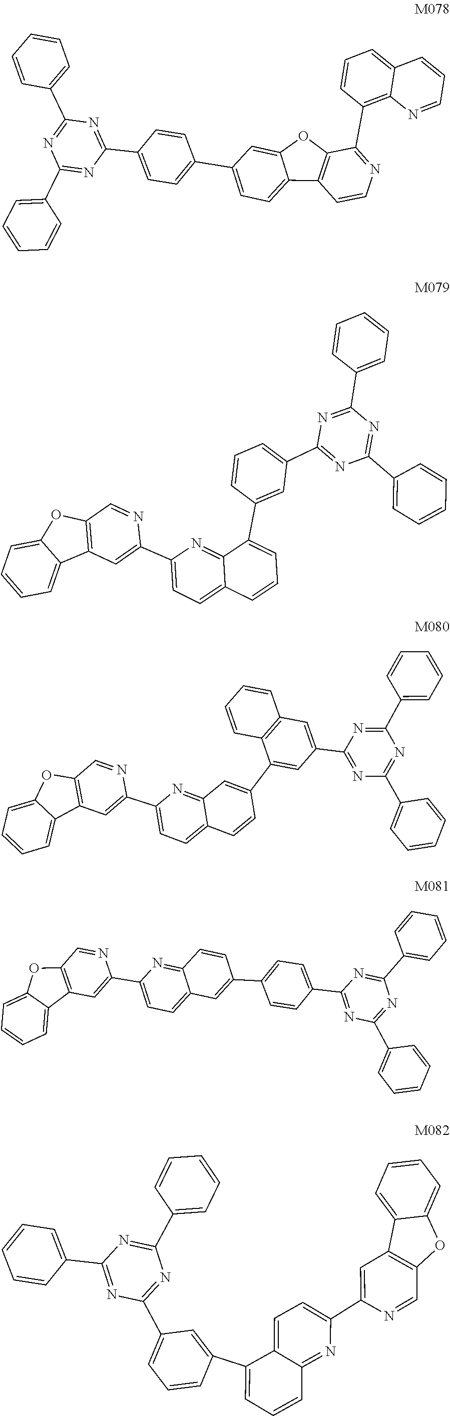 [SMILES: c1ccc(-c2nc(-c3ccccc3)nc(-c3ccc(-c4ccc5c(c4)oc4c(-c6cccc7cccnc67)nccc45)cc3)n2)cc1 ; c1ccc(-c2nc(-c3ccccc3)nc(-c3cccc(-c4cccc5ccc(-c6cc7c(cn6)oc6ccccc67)nc45)c3)n2)cc1 ; c1ccc(-c2nc(-c3ccccc3)nc(-c3cc(-c4ccc5ccc(-c6cc7c(cn6)oc6ccccc67)nc5c4)c4ccccc4c3)n2)cc1 ; c1ccc(-c2nc(-c3ccccc3)nc(-c3ccc(-c4ccc5nc(-c6cc7c(cn6)oc6ccccc67)ccc5c4)cc3)n2)cc1 ; c1ccc(-c2nc(-c3ccccc3)nc(-c3cccc(-c4cccc5nc(-c6cc7c(cn6)oc6ccccc67)ccc45)c3)n2)cc1]

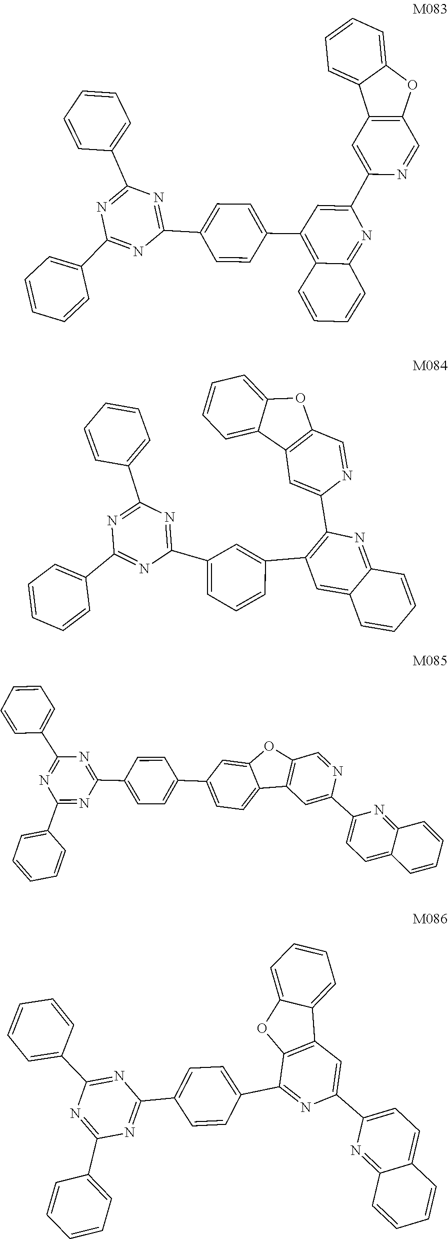 [SMILES: c1ccc(-c2nc(-c3ccccc3)nc(-c3ccc(-c4cc(-c5cc6c(cn5)oc5ccccc56)nc5ccccc45)cc3)n2)cc1 ; c1ccc(-c2nc(-c3ccccc3)nc(-c3cccc(-c4cc5ccccc5nc4-c4cc5c(cn4)oc4ccccc45)c3)n2)cc1 ; c1ccc(-c2nc(-c3ccccc3)nc(-c3ccc(-c4ccc5c(c4)oc4cnc(-c6ccc7ccccc7n6)cc45)cc3)n2)cc1 ; c1ccc(-c2nc(-c3ccccc3)nc(-c3ccc(-c4nc(-c5ccc6ccccc6n5)cc5c4oc4ccccc45)cc3)n2)cc1]

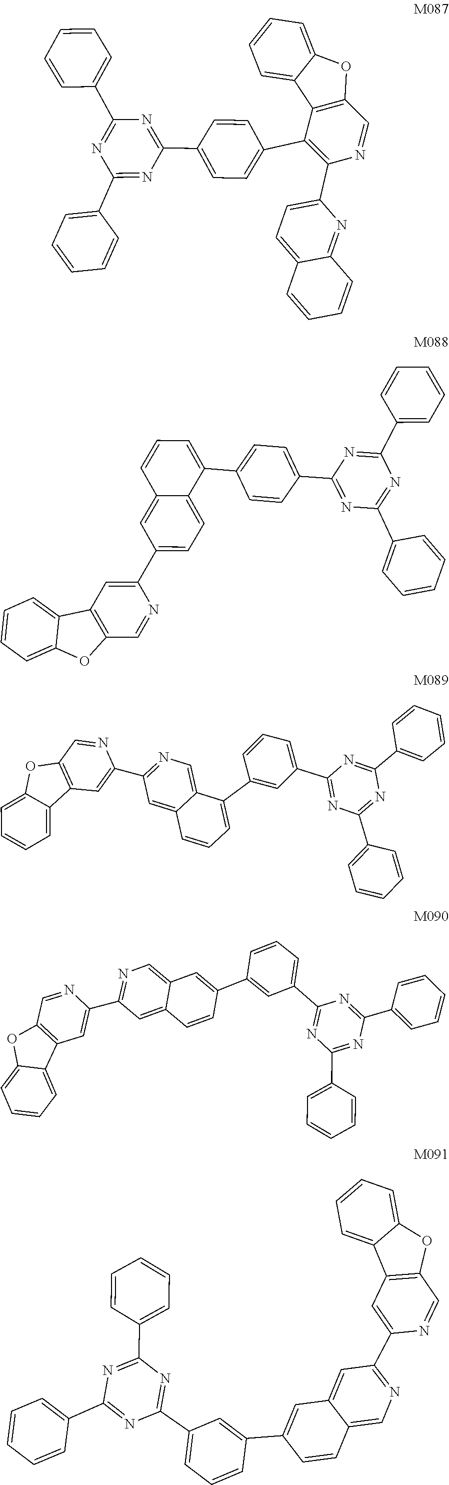 [SMILES: c1ccc(-c2nc(-c3ccccc3)nc(-c3ccc(-c4c(-c5ccc6ccccc6n5)ncc5oc6ccccc6c45)cc3)n2)cc1 ; c1ccc(-c2nc(-c3ccccc3)nc(-c3ccc(-c4cccc5cc(-c6cc7c(cn6)oc6ccccc67)ccc45)cc3)n2)cc1 ; c1ccc(-c2nc(-c3ccccc3)nc(-c3cccc(-c4cccc5cc(-c6cc7c(cn6)oc6ccccc67)ncc45)c3)n2)cc1 ; c1ccc(-c2nc(-c3ccccc3)nc(-c3cccc(-c4ccc5cc(-c6cc7c(cn6)oc6ccccc67)ncc5c4)c3)n2)cc1 ; c1ccc(-c2nc(-c3ccccc3)nc(-c3cccc(-c4ccc5cnc(-c6cc7c(cn6)oc6ccccc67)cc5c4)c3)n2)cc1]

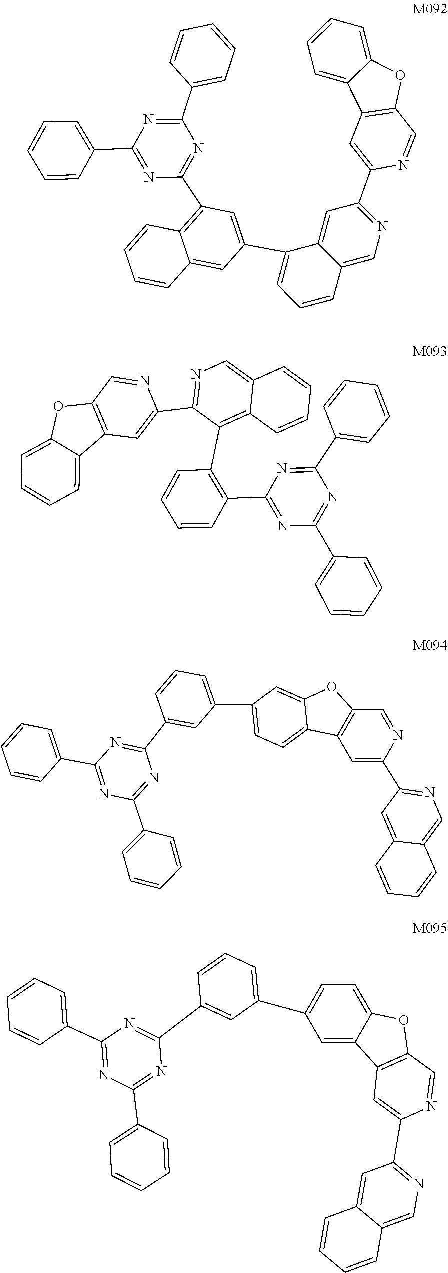 [SMILES: c1ccc(-c2nc(-c3ccccc3)nc(-c3cc(-c4cccc5cnc(-c6cc7c(cn6)oc6ccccc67)cc45)cc4ccccc34)n2)cc1 ; c1ccc(-c2nc(-c3ccccc3)nc(-c3ccccc3-c3c(-c4cc5c(cn4)oc4ccccc45)ncc4ccccc34)n2)cc1 ; c1ccc(-c2nc(-c3ccccc3)nc(-c3cccc(-c4ccc5c(c4)oc4cnc(-c6cc7ccccc7cn6)cc45)c3)n2)cc1 ; c1ccc(-c2nc(-c3ccccc3)nc(-c3cccc(-c4ccc5oc6cnc(-c7cc8ccccc8cn7)cc6c5c4)c3)n2)cc1]

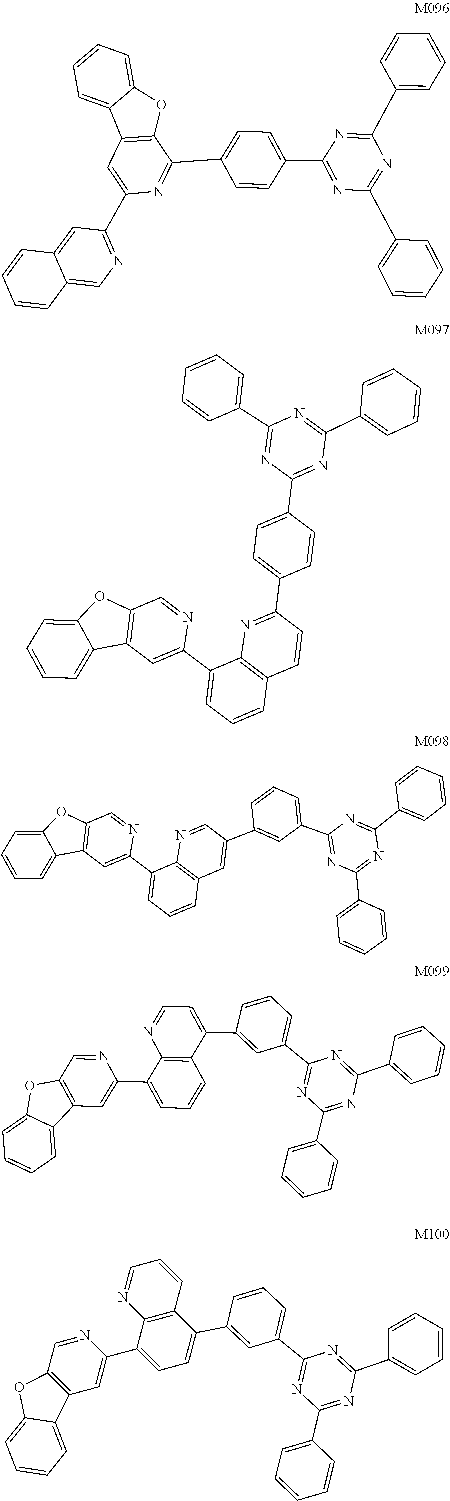 [SMILES: c1ccc(-c2nc(-c3ccccc3)nc(-c3ccc(-c4nc(-c5cc6ccccc6cn5)cc5c4oc4ccccc45)cc3)n2)cc1 ; c1ccc(-c2nc(-c3ccccc3)nc(-c3ccc(-c4ccc5cccc(-c6cc7c(cn6)oc6ccccc67)c5n4)cc3)n2)cc1 ; c1ccc(-c2nc(-c3ccccc3)nc(-c3cccc(-c4cnc5c(-c6cc7c(cn6)oc6ccccc67)cccc5c4)c3)n2)cc1 ; c1ccc(-c2nc(-c3ccccc3)nc(-c3cccc(-c4ccnc5c(-c6cc7c(cn6)oc6ccccc67)cccc45)c3)n2)cc1 ; c1ccc(-c2nc(-c3ccccc3)nc(-c3cccc(-c4ccc(-c5cc6c(cn5)oc5ccccc56)c5ncccc45)c3)n2)cc1]

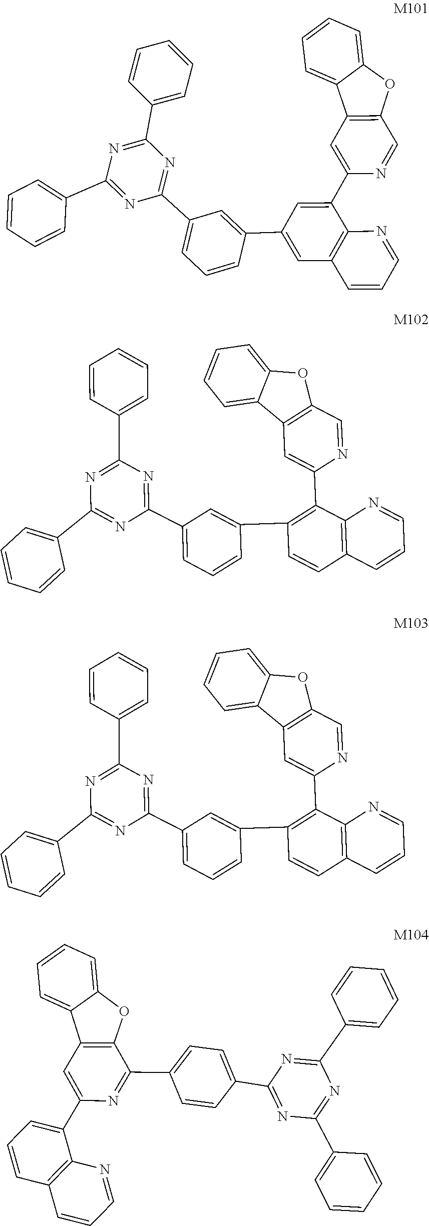 [SMILES: c1ccc(-c2nc(-c3ccccc3)nc(-c3cccc(-c4cc(-c5cc6c(cn5)oc5ccccc56)c5ncccc5c4)c3)n2)cc1 ; c1ccc(-c2nc(-c3ccccc3)nc(-c3cccc(-c4ccc5cccnc5c4-c4cc5c(cn4)oc4ccccc45)c3)n2)cc1 ; c1ccc(-c2nc(-c3ccccc3)nc(-c3cccc(-c4ccc5cccnc5c4-c4cc5c(cn4)oc4ccccc45)c3)n2)cc1 ; c1ccc(-c2nc(-c3ccccc3)nc(-c3ccc(-c4nc(-c5cccc6cccnc56)cc5c4oc4ccccc45)cc3)n2)cc1]

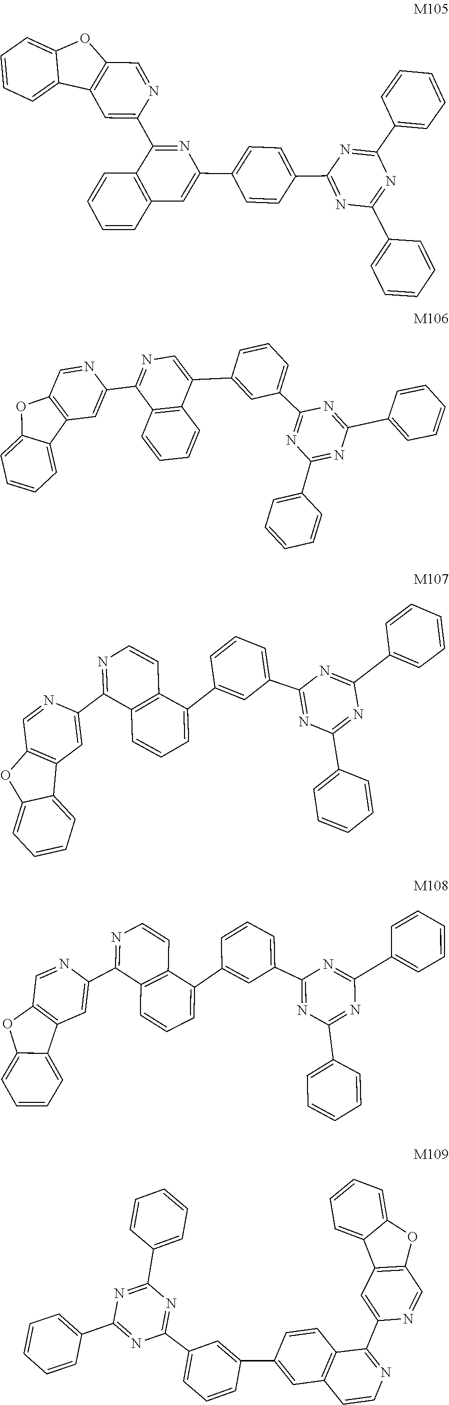 [SMILES: c1ccc(-c2nc(-c3ccccc3)nc(-c3ccc(-c4cc5ccccc5c(-c5cc6c(cn5)oc5ccccc56)n4)cc3)n2)cc1 ; c1ccc(-c2nc(-c3ccccc3)nc(-c3cccc(-c4cnc(-c5cc6c(cn5)oc5ccccc56)c5ccccc45)c3)n2)cc1 ; c1ccc(-c2nc(-c3ccccc3)nc(-c3cccc(-c4cccc5c(-c6cc7c(cn6)oc6ccccc67)nccc45)c3)n2)cc1 ; c1ccc(-c2nc(-c3ccccc3)nc(-c3cccc(-c4cccc5c(-c6cc7c(cn6)oc6ccccc67)nccc45)c3)n2)cc1 ; c1ccc(-c2nc(-c3ccccc3)nc(-c3cccc(-c4ccc5c(-c6cc7c(cn6)oc6ccccc67)nccc5c4)c3)n2)cc1]

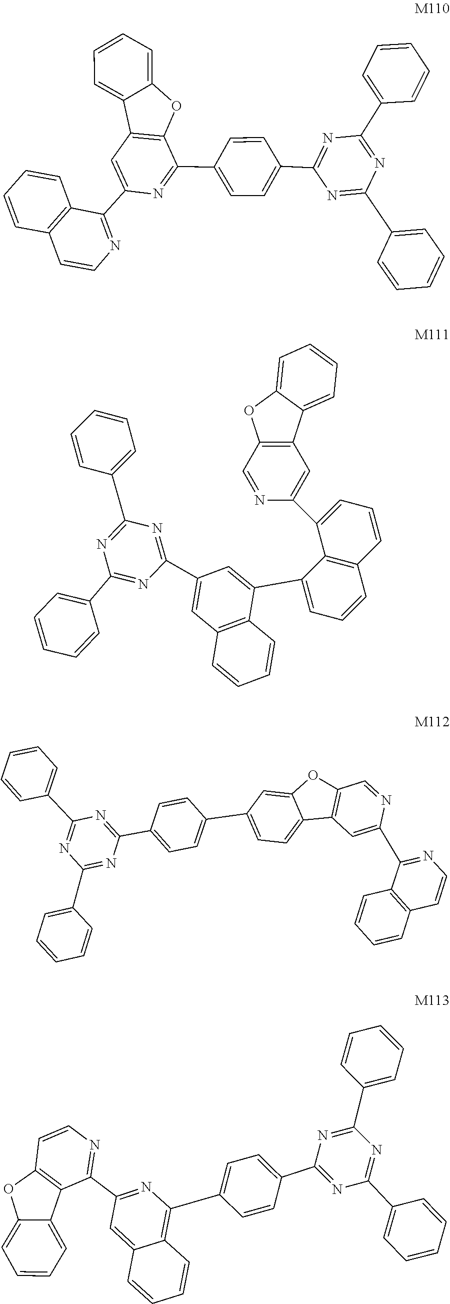 [SMILES: c1ccc(-c2nc(-c3ccccc3)nc(-c3ccc(-c4nc(-c5nccc6ccccc56)cc5c4oc4ccccc45)cc3)n2)cc1 ; c1ccc(-c2nc(-c3ccccc3)nc(-c3cc(-c4cccc5cccc(-c6cc7c(cn6)oc6ccccc67)c45)c4ccccc4c3)n2)cc1 ; c1ccc(-c2nc(-c3ccccc3)nc(-c3ccc(-c4ccc5c(c4)oc4cnc(-c6nccc7ccccc67)cc45)cc3)n2)cc1 ; c1ccc(-c2nc(-c3ccccc3)nc(-c3ccc(-c4nc(-c5nccc6oc7ccccc7c56)cc5ccccc45)cc3)n2)cc1]

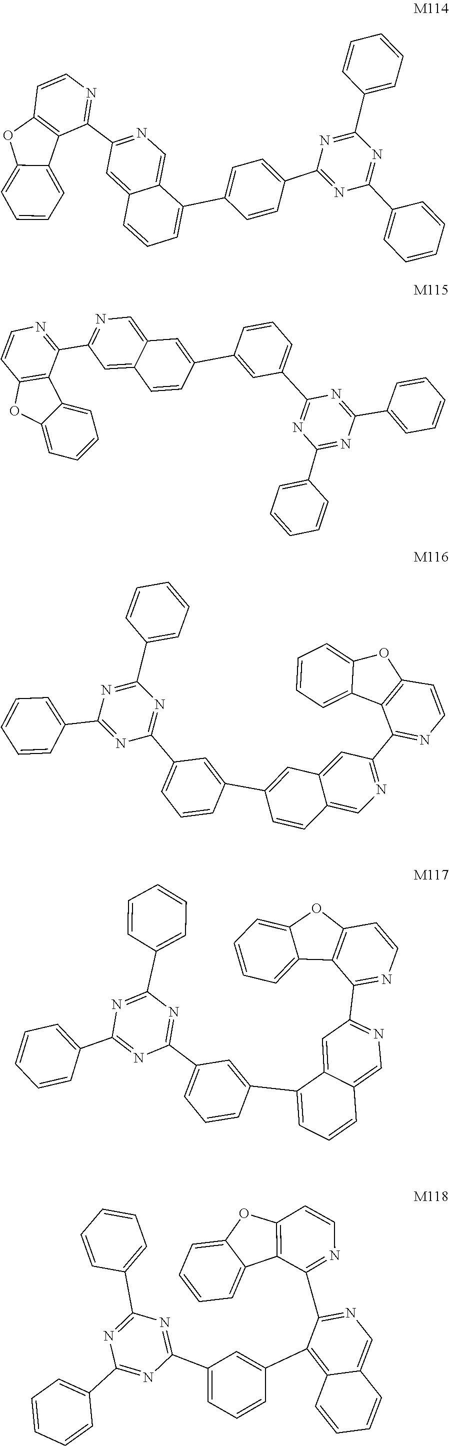 [SMILES: c1ccc(-c2nc(-c3ccccc3)nc(-c3ccc(-c4cccc5cc(-c6nccc7oc8ccccc8c67)ncc45)cc3)n2)cc1 ; c1ccc(-c2nc(-c3ccccc3)nc(-c3cccc(-c4ccc5cc(-c6nccc7oc8ccccc8c67)ncc5c4)c3)n2)cc1 ; c1ccc(-c2nc(-c3ccccc3)nc(-c3cccc(-c4ccc5cnc(-c6nccc7oc8ccccc8c67)cc5c4)c3)n2)cc1 ; c1ccc(-c2nc(-c3ccccc3)nc(-c3cccc(-c4cccc5cnc(-c6nccc7oc8ccccc8c67)cc45)c3)n2)cc1 ; c1ccc(-c2nc(-c3ccccc3)nc(-c3cccc(-c4c(-c5nccc6oc7ccccc7c56)ncc5ccccc45)c3)n2)cc1]

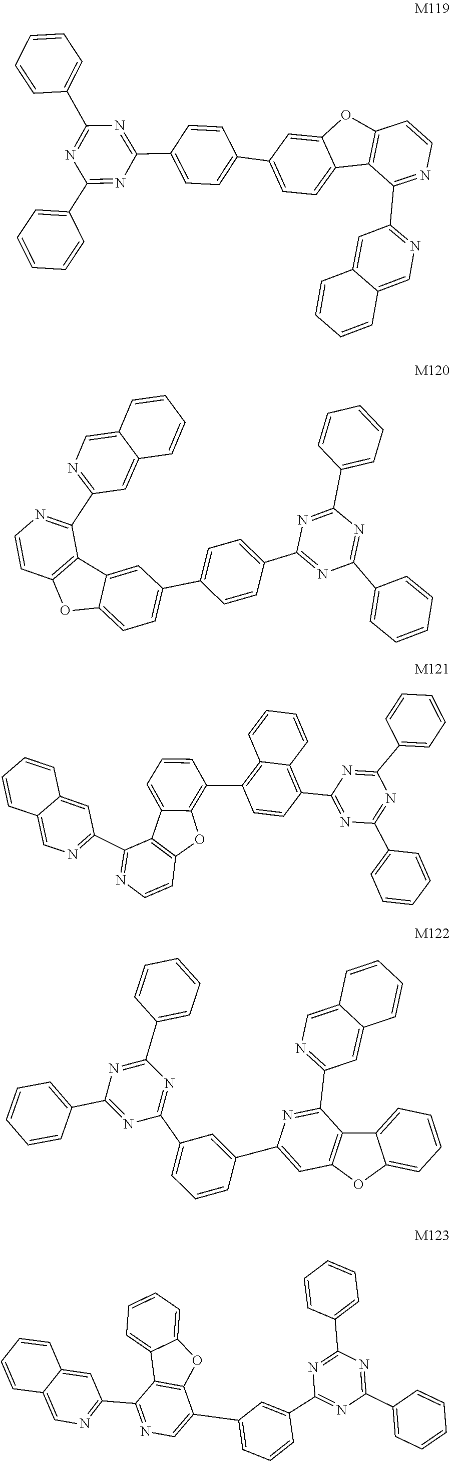 [SMILES: c1ccc(-c2nc(-c3ccccc3)nc(-c3ccc(-c4ccc5c(c4)oc4ccnc(-c6cc7ccccc7cn6)c45)cc3)n2)cc1 ; c1ccc(-c2nc(-c3ccccc3)nc(-c3ccc(-c4ccc5oc6ccnc(-c7cc8ccccc8cn7)c6c5c4)cc3)n2)cc1 ; c1ccc(-c2nc(-c3ccccc3)nc(-c3ccc(-c4cccc5c4oc4ccnc(-c6cc7ccccc7cn6)c45)c4ccccc34)n2)cc1 ; c1ccc(-c2nc(-c3ccccc3)nc(-c3cccc(-c4cc5oc6ccccc6c5c(-c5cc6ccccc6cn5)n4)c3)n2)cc1 ; c1ccc(-c2nc(-c3ccccc3)nc(-c3cccc(-c4cnc(-c5cc6ccccc6cn5)c5c4oc4ccccc45)c3)n2)cc1]

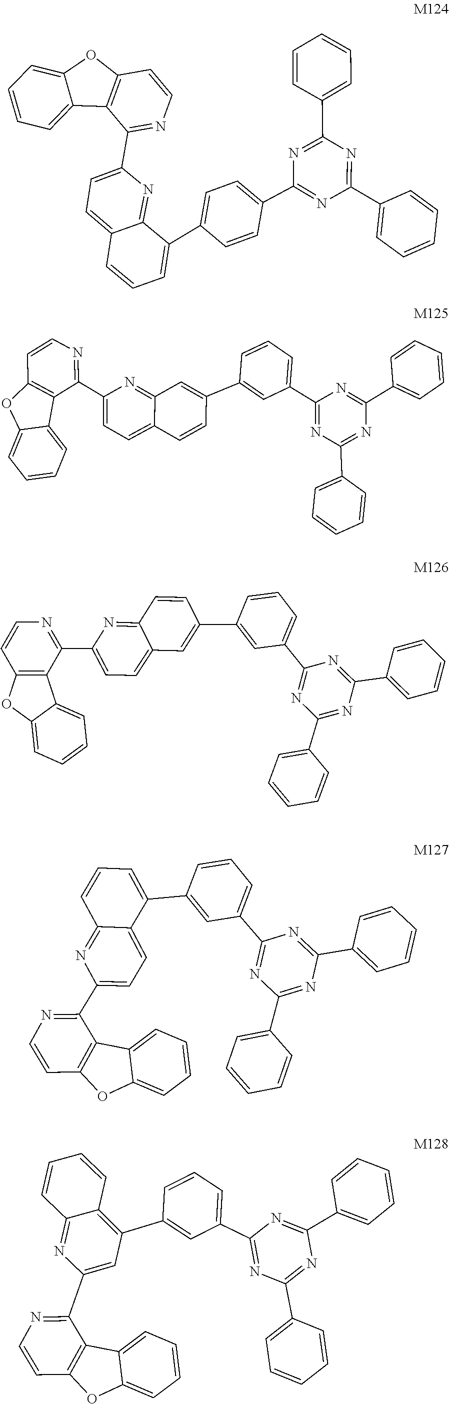 [SMILES: c1ccc(-c2nc(-c3ccccc3)nc(-c3ccc(-c4cccc5ccc(-c6nccc7oc8ccccc8c67)nc45)cc3)n2)cc1 ; c1ccc(-c2nc(-c3ccccc3)nc(-c3cccc(-c4ccc5ccc(-c6nccc7oc8ccccc8c67)nc5c4)c3)n2)cc1 ; c1ccc(-c2nc(-c3ccccc3)nc(-c3cccc(-c4ccc5nc(-c6nccc7oc8ccccc8c67)ccc5c4)c3)n2)cc1 ; c1ccc(-c2nc(-c3ccccc3)nc(-c3cccc(-c4cccc5nc(-c6nccc7oc8ccccc8c67)ccc45)c3)n2)cc1 ; c1ccc(-c2nc(-c3ccccc3)nc(-c3cccc(-c4cc(-c5nccc6oc7ccccc7c56)nc5ccccc45)c3)n2)cc1]

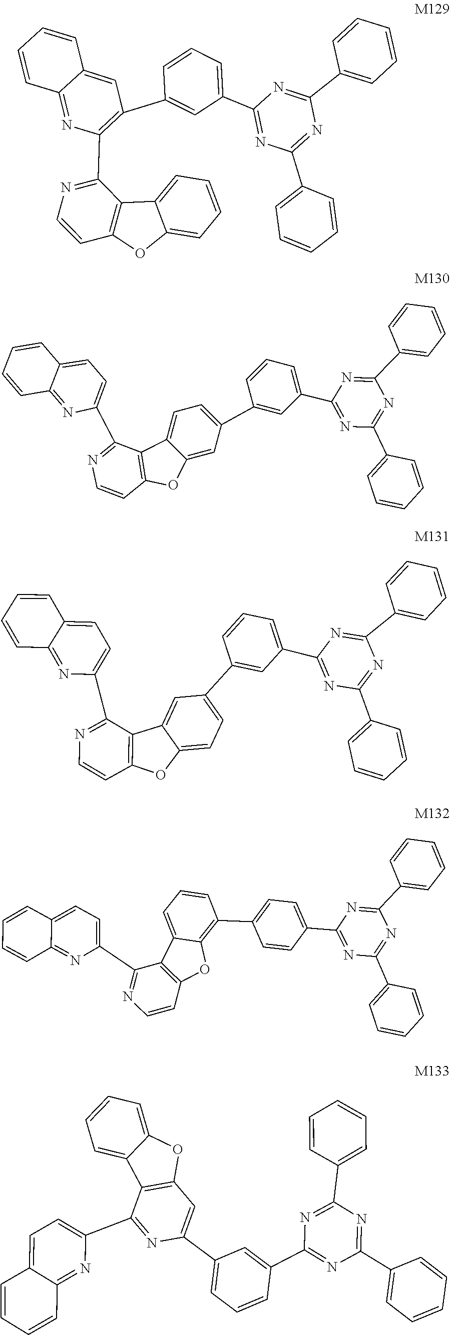 [SMILES: c1ccc(-c2nc(-c3ccccc3)nc(-c3cccc(-c4cc5ccccc5nc4-c4nccc5oc6ccccc6c45)c3)n2)cc1 ; c1ccc(-c2nc(-c3ccccc3)nc(-c3cccc(-c4ccc5c(c4)oc4ccnc(-c6ccc7ccccc7n6)c45)c3)n2)cc1 ; c1ccc(-c2nc(-c3ccccc3)nc(-c3cccc(-c4ccc5oc6ccnc(-c7ccc8ccccc8n7)c6c5c4)c3)n2)cc1 ; c1ccc(-c2nc(-c3ccccc3)nc(-c3ccc(-c4cccc5c4oc4ccnc(-c6ccc7ccccc7n6)c45)cc3)n2)cc1 ; c1ccc(-c2nc(-c3ccccc3)nc(-c3cccc(-c4cc5oc6ccccc6c5c(-c5ccc6ccccc6n5)n4)c3)n2)cc1]

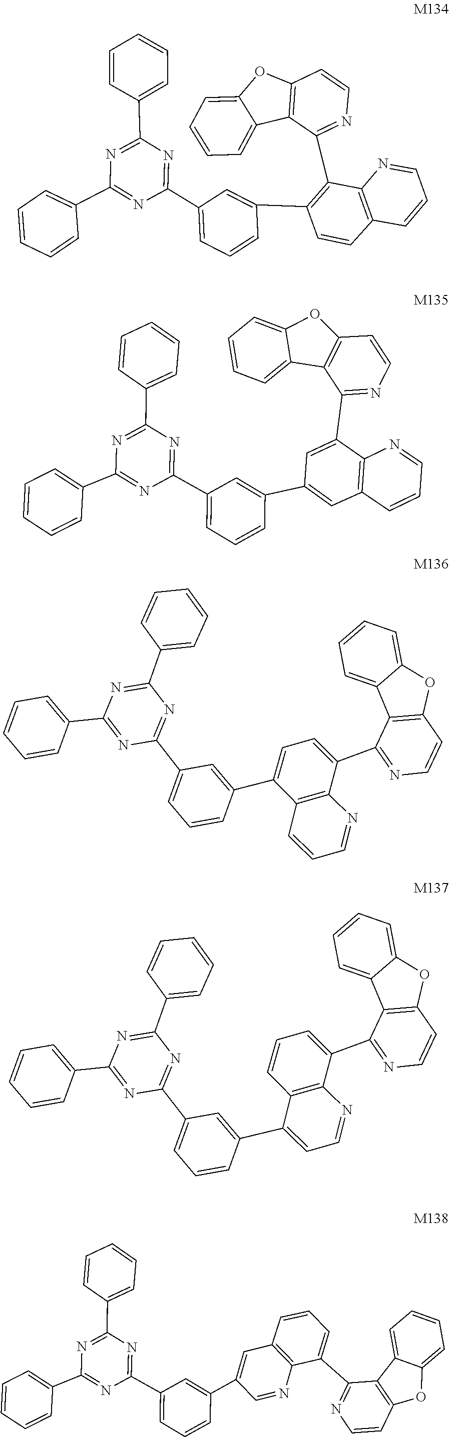 [SMILES: c1ccc(-c2nc(-c3ccccc3)nc(-c3cccc(-c4ccc5cccnc5c4-c4nccc5oc6ccccc6c45)c3)n2)cc1 ; c1ccc(-c2nc(-c3ccccc3)nc(-c3cccc(-c4cc(-c5nccc6oc7ccccc7c56)c5ncccc5c4)c3)n2)cc1 ; c1ccc(-c2nc(-c3ccccc3)nc(-c3cccc(-c4ccc(-c5nccc6oc7ccccc7c56)c5ncccc45)c3)n2)cc1 ; c1ccc(-c2nc(-c3ccccc3)nc(-c3cccc(-c4ccnc5c(-c6nccc7oc8ccccc8c67)cccc45)c3)n2)cc1 ; c1ccc(-c2nc(-c3ccccc3)nc(-c3cccc(-c4cnc5c(-c6nccc7oc8ccccc8c67)cccc5c4)c3)n2)cc1]

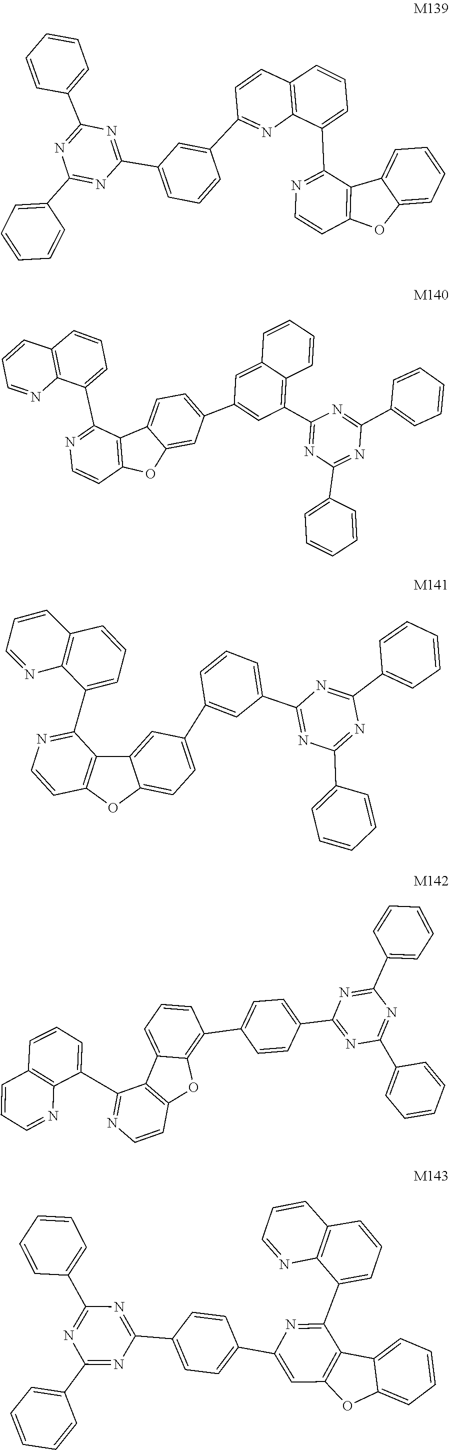 [SMILES: c1ccc(-c2nc(-c3ccccc3)nc(-c3cccc(-c4ccc5cccc(-c6nccc7oc8ccccc8c67)c5n4)c3)n2)cc1 ; c1ccc(-c2nc(-c3ccccc3)nc(-c3cc(-c4ccc5c(c4)oc4ccnc(-c6cccc7cccnc67)c45)cc4ccccc34)n2)cc1 ; c1ccc(-c2nc(-c3ccccc3)nc(-c3cccc(-c4ccc5oc6ccnc(-c7cccc8cccnc78)c6c5c4)c3)n2)cc1 ; c1ccc(-c2nc(-c3ccccc3)nc(-c3ccc(-c4cccc5c4oc4ccnc(-c6cccc7cccnc67)c45)cc3)n2)cc1 ; c1ccc(-c2nc(-c3ccccc3)nc(-c3ccc(-c4cc5oc6ccccc6c5c(-c5cccc6cccnc56)n4)cc3)n2)cc1]

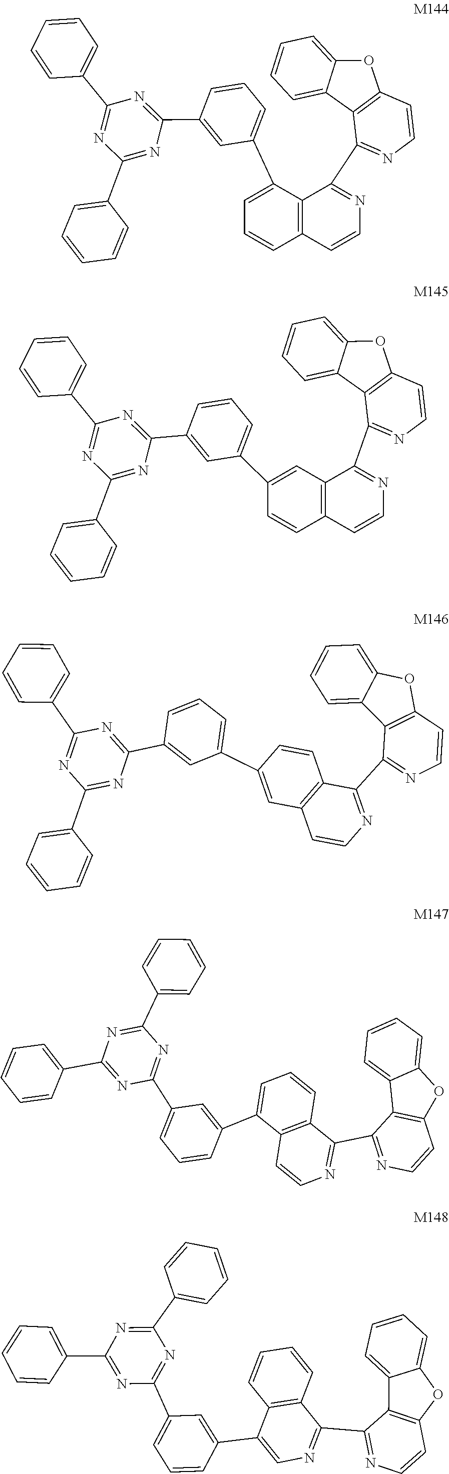 [SMILES: c1ccc(-c2nc(-c3ccccc3)nc(-c3cccc(-c4cccc5ccnc(-c6nccc7oc8ccccc8c67)c45)c3)n2)cc1 ; c1ccc(-c2nc(-c3ccccc3)nc(-c3cccc(-c4ccc5ccnc(-c6nccc7oc8ccccc8c67)c5c4)c3)n2)cc1 ; c1ccc(-c2nc(-c3ccccc3)nc(-c3cccc(-c4ccc5c(-c6nccc7oc8ccccc8c67)nccc5c4)c3)n2)cc1 ; c1ccc(-c2nc(-c3ccccc3)nc(-c3cccc(-c4cccc5c(-c6nccc7oc8ccccc8c67)nccc45)c3)n2)cc1 ; c1ccc(-c2nc(-c3ccccc3)nc(-c3cccc(-c4cnc(-c5nccc6oc7ccccc7c56)c5ccccc45)c3)n2)cc1]

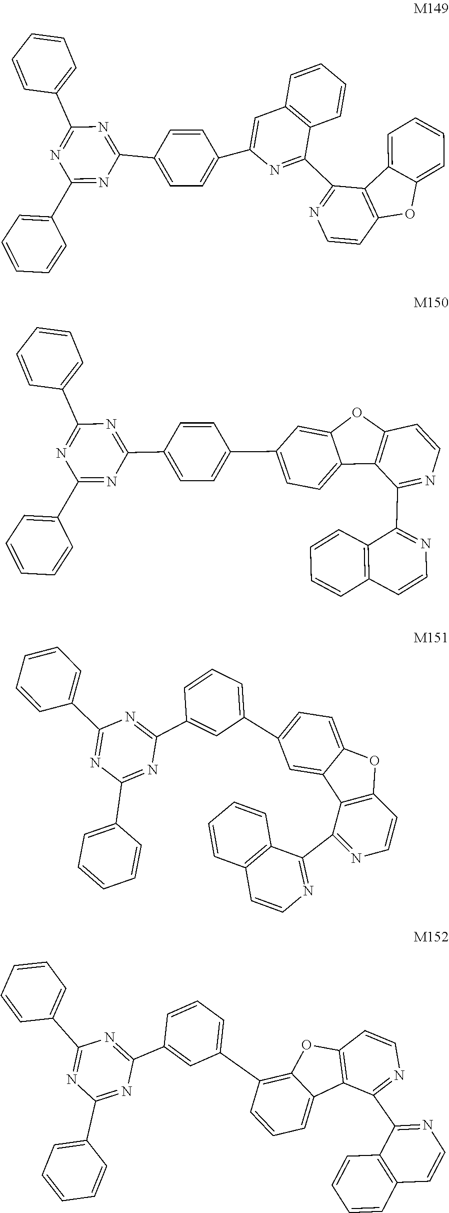 [SMILES: c1ccc(-c2nc(-c3ccccc3)nc(-c3ccc(-c4cc5ccccc5c(-c5nccc6oc7ccccc7c56)n4)cc3)n2)cc1 ; c1ccc(-c2nc(-c3ccccc3)nc(-c3ccc(-c4ccc5c(c4)oc4ccnc(-c6nccc7ccccc67)c45)cc3)n2)cc1 ; c1ccc(-c2nc(-c3ccccc3)nc(-c3cccc(-c4ccc5oc6ccnc(-c7nccc8ccccc78)c6c5c4)c3)n2)cc1 ; c1ccc(-c2nc(-c3ccccc3)nc(-c3cccc(-c4cccc5c4oc4ccnc(-c6nccc7ccccc67)c45)c3)n2)cc1]

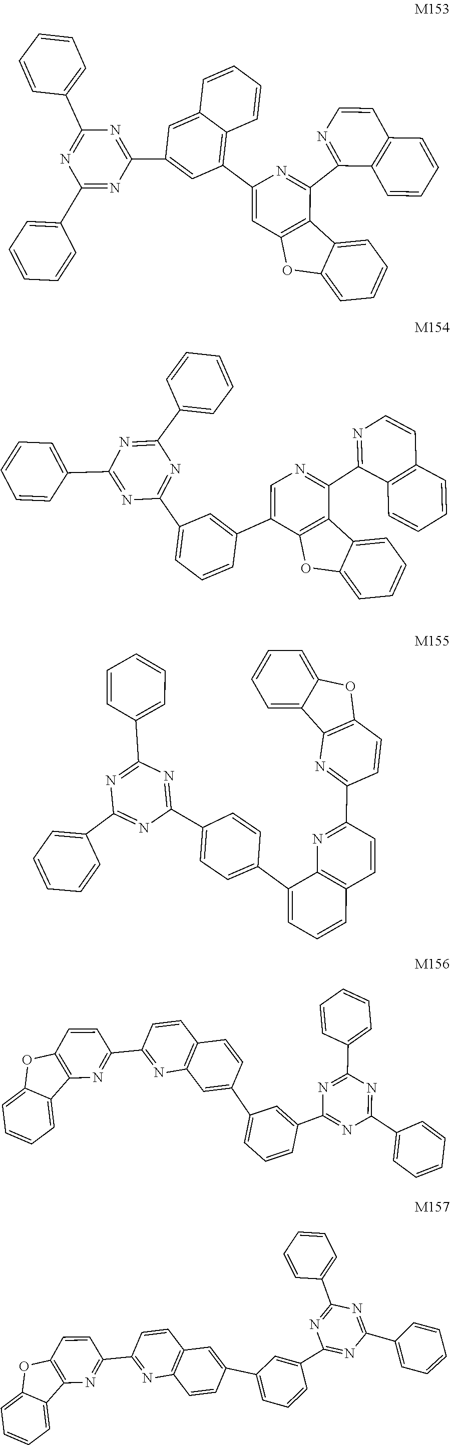 [SMILES: c1ccc(-c2nc(-c3ccccc3)nc(-c3cc(-c4cc5oc6ccccc6c5c(-c5nccc6ccccc56)n4)c4ccccc4c3)n2)cc1 ; c1ccc(-c2nc(-c3ccccc3)nc(-c3cccc(-c4cnc(-c5nccc6ccccc56)c5c4oc4ccccc45)c3)n2)cc1 ; c1ccc(-c2nc(-c3ccccc3)nc(-c3ccc(-c4cccc5ccc(-c6ccc7oc8ccccc8c7n6)nc45)cc3)n2)cc1 ; c1ccc(-c2nc(-c3ccccc3)nc(-c3cccc(-c4ccc5ccc(-c6ccc7oc8ccccc8c7n6)nc5c4)c3)n2)cc1 ; c1ccc(-c2nc(-c3ccccc3)nc(-c3cccc(-c4ccc5nc(-c6ccc7oc8ccccc8c7n6)ccc5c4)c3)n2)cc1]

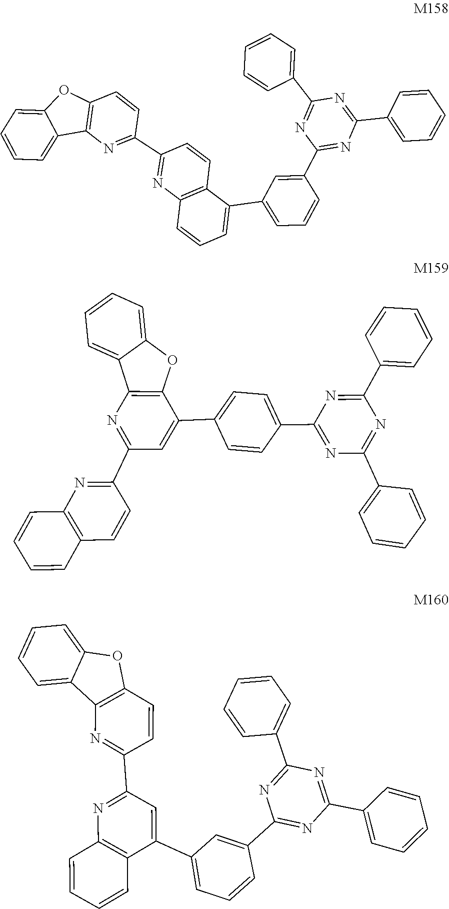 [SMILES: c1ccc(-c2nc(-c3ccccc3)nc(-c3cccc(-c4cccc5nc(-c6ccc7oc8ccccc8c7n6)ccc45)c3)n2)cc1 ; c1ccc(-c2nc(-c3ccccc3)nc(-c3ccc(-c4cc(-c5ccc6ccccc6n5)nc5c4oc4ccccc45)cc3)n2)cc1 ; c1ccc(-c2nc(-c3ccccc3)nc(-c3cccc(-c4cc(-c5ccc6oc7ccccc7c6n5)nc5ccccc45)c3)n2)cc1]

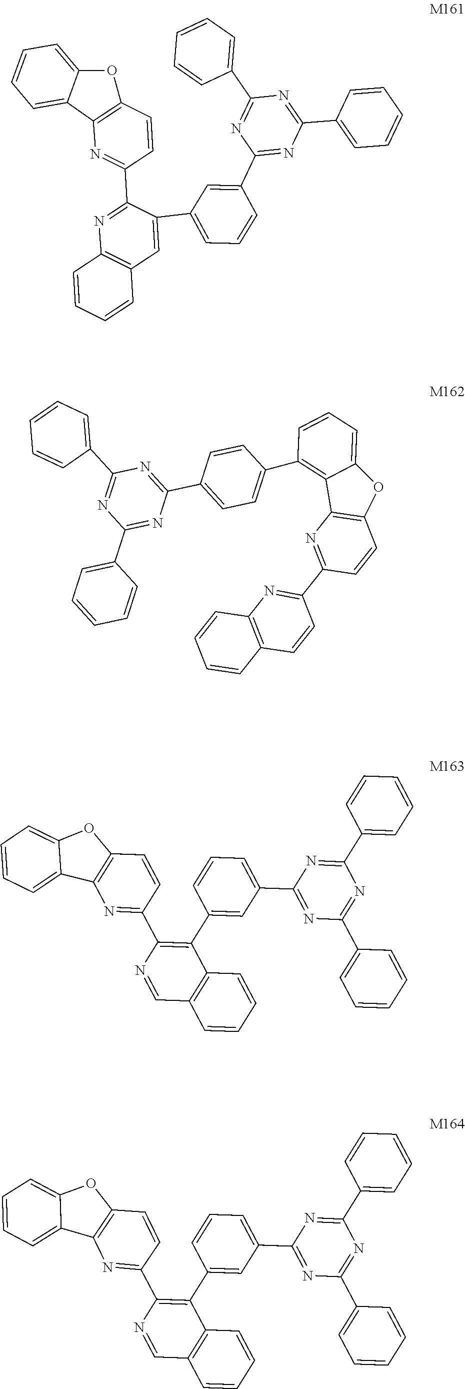 [SMILES: c1ccc(-c2nc(-c3ccccc3)nc(-c3cccc(-c4cc5ccccc5nc4-c4ccc5oc6ccccc6c5n4)c3)n2)cc1 ; c1ccc(-c2nc(-c3ccccc3)nc(-c3ccc(-c4cccc5oc6ccc(-c7ccc8ccccc8n7)nc6c45)cc3)n2)cc1 ; c1ccc(-c2nc(-c3ccccc3)nc(-c3cccc(-c4c(-c5ccc6oc7ccccc7c6n5)ncc5ccccc45)c3)n2)cc1 ; c1ccc(-c2nc(-c3ccccc3)nc(-c3cccc(-c4c(-c5ccc6oc7ccccc7c6n5)ncc5ccccc45)c3)n2)cc1]

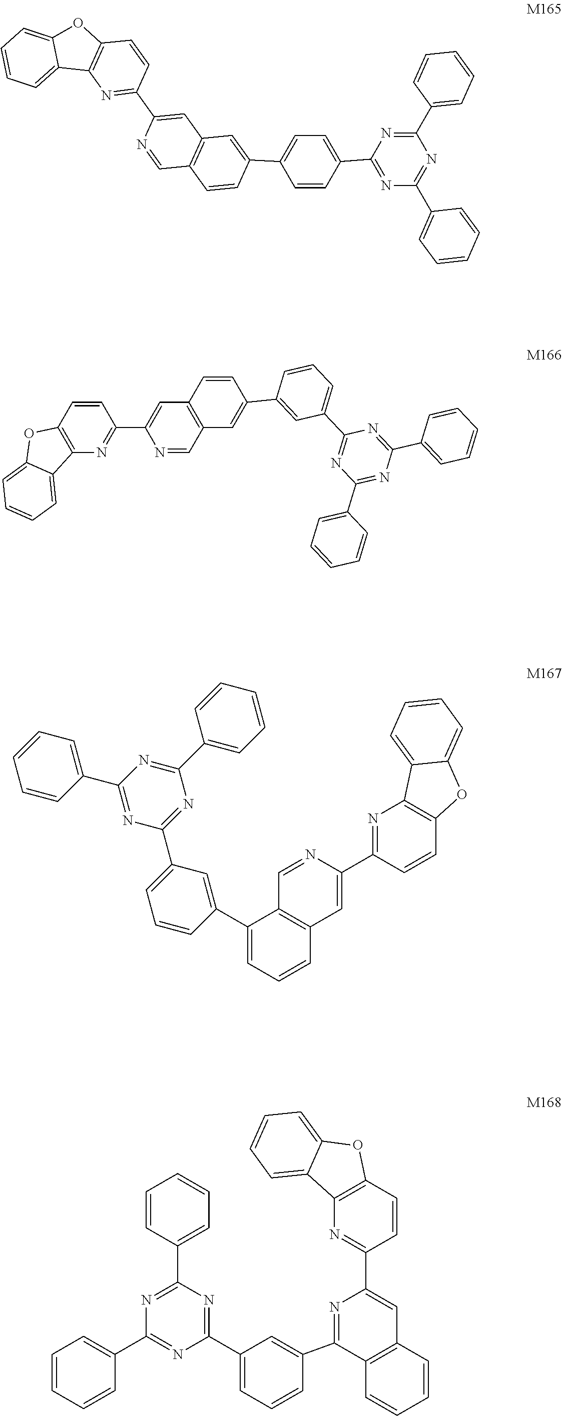 [SMILES: c1ccc(-c2nc(-c3ccccc3)nc(-c3ccc(-c4ccc5cnc(-c6ccc7oc8ccccc8c7n6)cc5c4)cc3)n2)cc1 ; c1ccc(-c2nc(-c3ccccc3)nc(-c3cccc(-c4ccc5cc(-c6ccc7oc8ccccc8c7n6)ncc5c4)c3)n2)cc1 ; c1ccc(-c2nc(-c3ccccc3)nc(-c3cccc(-c4cccc5cc(-c6ccc7oc8ccccc8c7n6)ncc45)c3)n2)cc1 ; c1ccc(-c2nc(-c3ccccc3)nc(-c3cccc(-c4nc(-c5ccc6oc7ccccc7c6n5)cc5ccccc45)c3)n2)cc1]

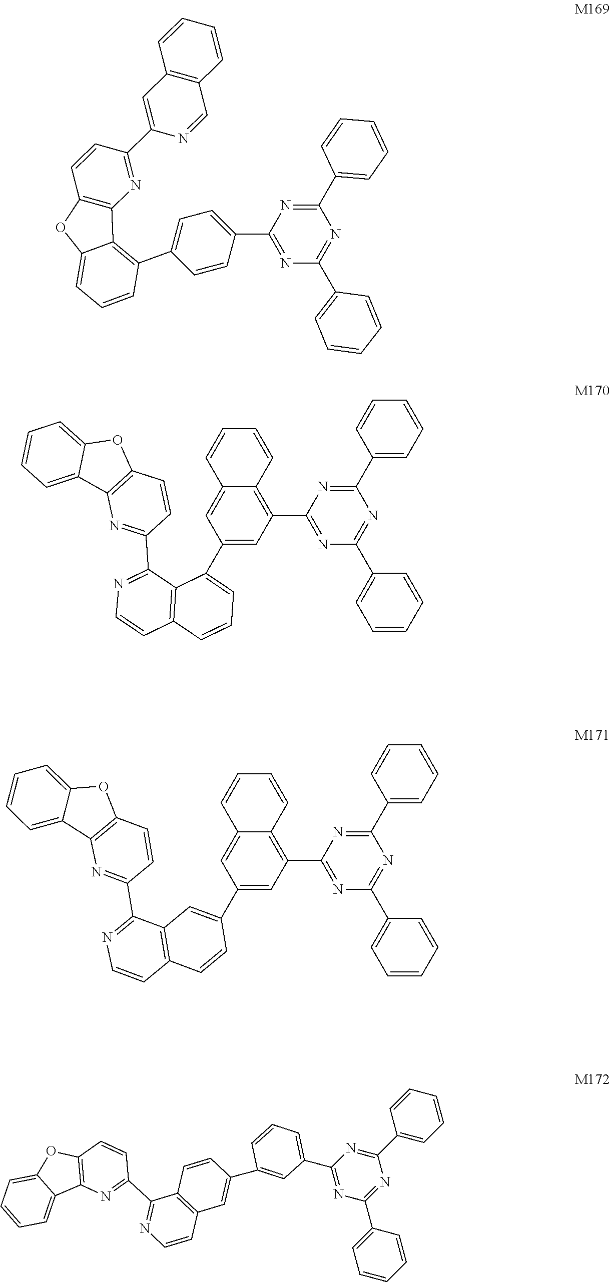 [SMILES: c1ccc(-c2nc(-c3ccccc3)nc(-c3ccc(-c4cccc5oc6ccc(-c7cc8ccccc8cn7)nc6c45)cc3)n2)cc1 ; c1ccc(-c2nc(-c3ccccc3)nc(-c3cc(-c4cccc5ccnc(-c6ccc7oc8ccccc8c7n6)c45)cc4ccccc34)n2)cc1 ; c1ccc(-c2nc(-c3ccccc3)nc(-c3cc(-c4ccc5ccnc(-c6ccc7oc8ccccc8c7n6)c5c4)cc4ccccc34)n2)cc1 ; c1ccc(-c2nc(-c3ccccc3)nc(-c3cccc(-c4ccc5c(-c6ccc7oc8ccccc8c7n6)nccc5c4)c3)n2)cc1]

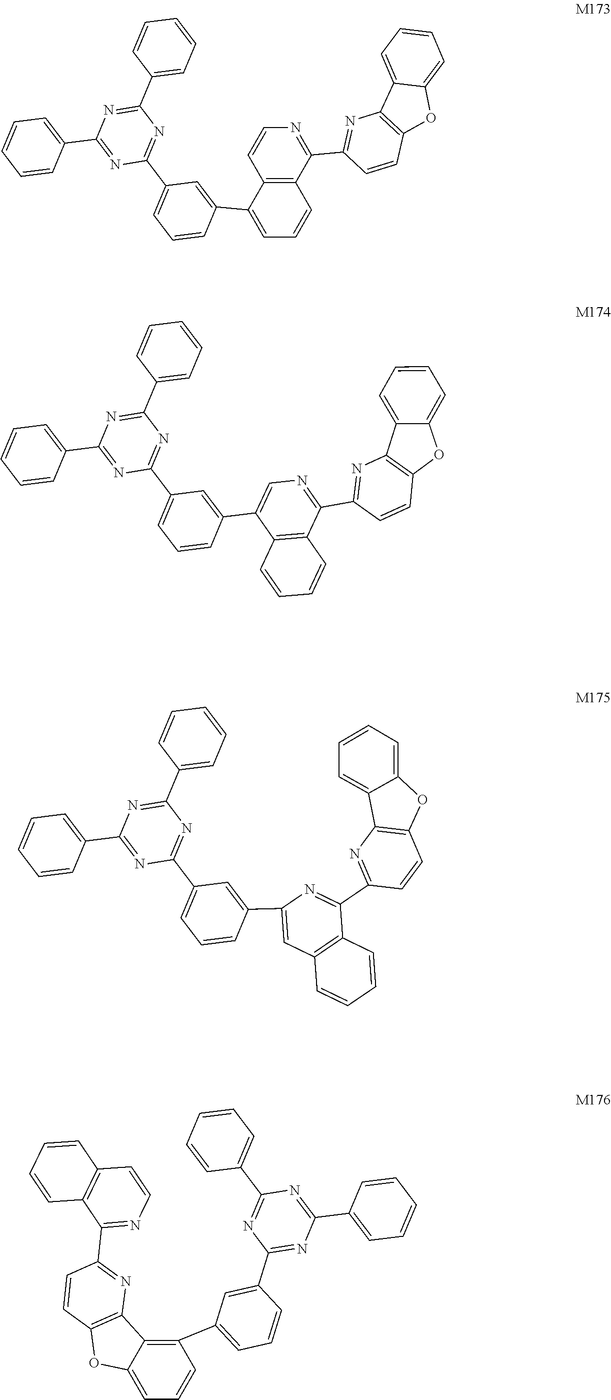 [SMILES: c1ccc(-c2nc(-c3ccccc3)nc(-c3cccc(-c4cccc5c(-c6ccc7oc8ccccc8c7n6)nccc45)c3)n2)cc1 ; c1ccc(-c2nc(-c3ccccc3)nc(-c3cccc(-c4cnc(-c5ccc6oc7ccccc7c6n5)c5ccccc45)c3)n2)cc1 ; c1ccc(-c2nc(-c3ccccc3)nc(-c3cccc(-c4cc5ccccc5c(-c5ccc6oc7ccccc7c6n5)n4)c3)n2)cc1 ; c1ccc(-c2nc(-c3ccccc3)nc(-c3cccc(-c4cccc5oc6ccc(-c7nccc8ccccc78)nc6c45)c3)n2)cc1]

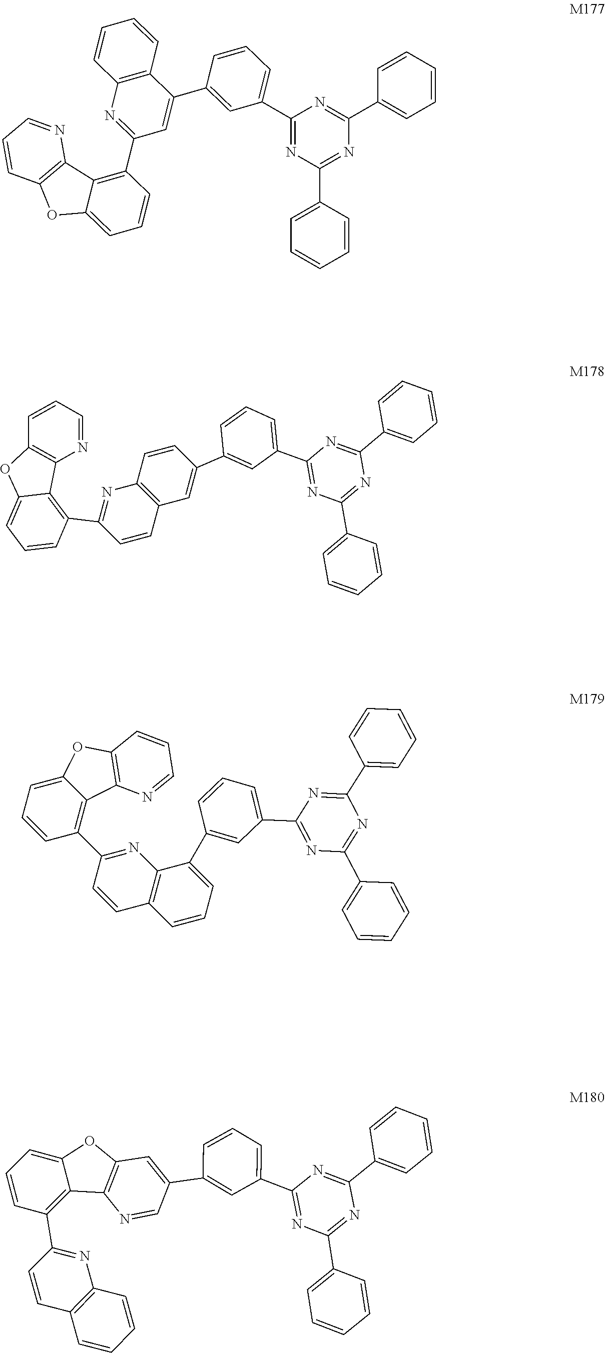 [SMILES: c1ccc(-c2nc(-c3ccccc3)nc(-c3cccc(-c4cc(-c5cccc6oc7cccnc7c56)nc5ccccc45)c3)n2)cc1 ; c1ccc(-c2nc(-c3ccccc3)nc(-c3cccc(-c4ccc5nc(-c6cccc7oc8cccnc8c67)ccc5c4)c3)n2)cc1 ; c1ccc(-c2nc(-c3ccccc3)nc(-c3cccc(-c4cccc5ccc(-c6cccc7oc8cccnc8c67)nc45)c3)n2)cc1 ; c1ccc(-c2nc(-c3ccccc3)nc(-c3cccc(-c4cnc5c(c4)oc4cccc(-c6ccc7ccccc7n6)c45)c3)n2)cc1]

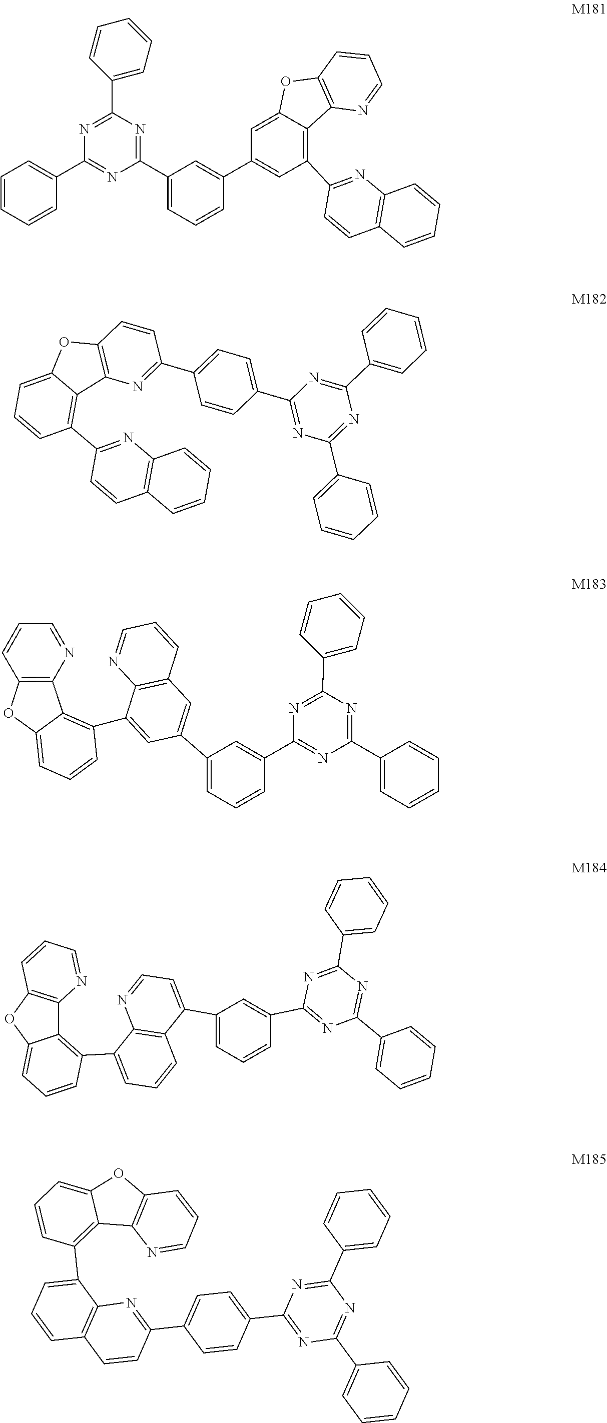 [SMILES: c1ccc(-c2nc(-c3ccccc3)nc(-c3cccc(-c4cc(-c5ccc6ccccc6n5)c5c(c4)oc4cccnc45)c3)n2)cc1 ; c1ccc(-c2nc(-c3ccccc3)nc(-c3ccc(-c4ccc5oc6cccc(-c7ccc8ccccc8n7)c6c5n4)cc3)n2)cc1 ; c1ccc(-c2nc(-c3ccccc3)nc(-c3cccc(-c4cc(-c5cccc6oc7cccnc7c56)c5ncccc5c4)c3)n2)cc1 ; c1ccc(-c2nc(-c3ccccc3)nc(-c3cccc(-c4ccnc5c(-c6cccc7oc8cccnc8c67)cccc45)c3)n2)cc1 ; c1ccc(-c2nc(-c3ccccc3)nc(-c3ccc(-c4ccc5cccc(-c6cccc7oc8cccnc8c67)c5n4)cc3)n2)cc1]

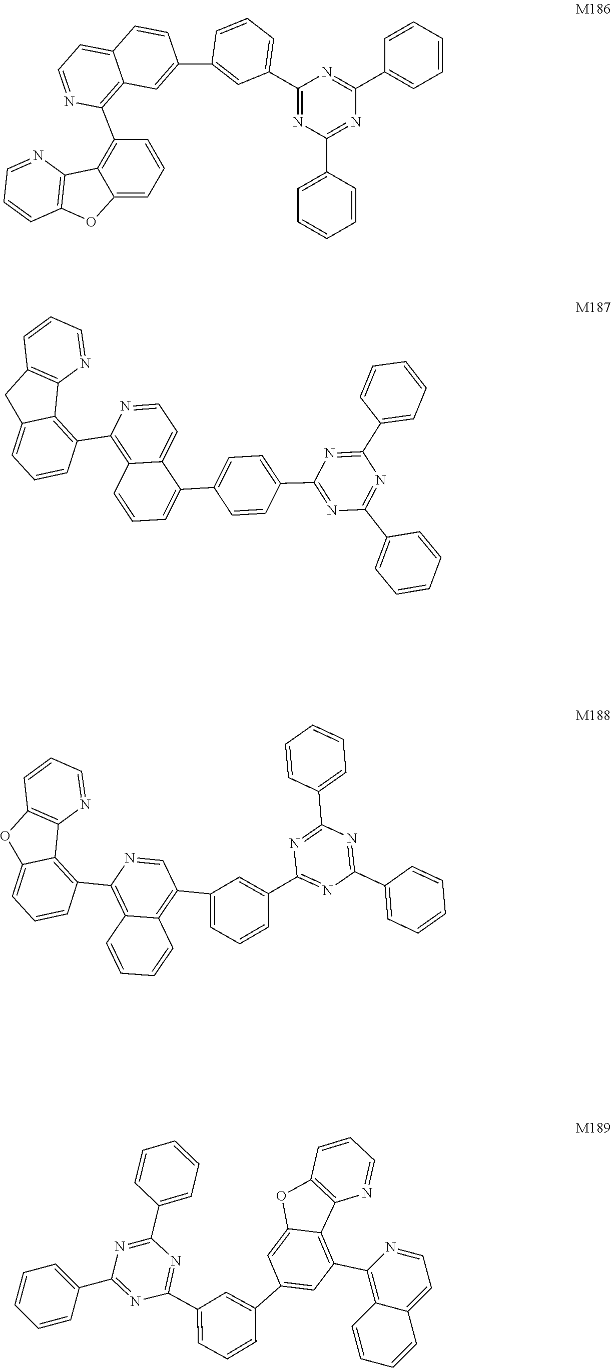 [SMILES: c1ccc(-c2nc(-c3ccccc3)nc(-c3cccc(-c4ccc5ccnc(-c6cccc7oc8cccnc8c67)c5c4)c3)n2)cc1 ; c1ccc(-c2nc(-c3ccccc3)nc(-c3ccc(-c4cccc5c(-c6cccc7c6-c6ncccc6C7)nccc45)cc3)n2)cc1 ; c1ccc(-c2nc(-c3ccccc3)nc(-c3cccc(-c4cnc(-c5cccc6oc7cccnc7c56)c5ccccc45)c3)n2)cc1 ; c1ccc(-c2nc(-c3ccccc3)nc(-c3cccc(-c4cc(-c5nccc6ccccc56)c5c(c4)oc4cccnc45)c3)n2)cc1]

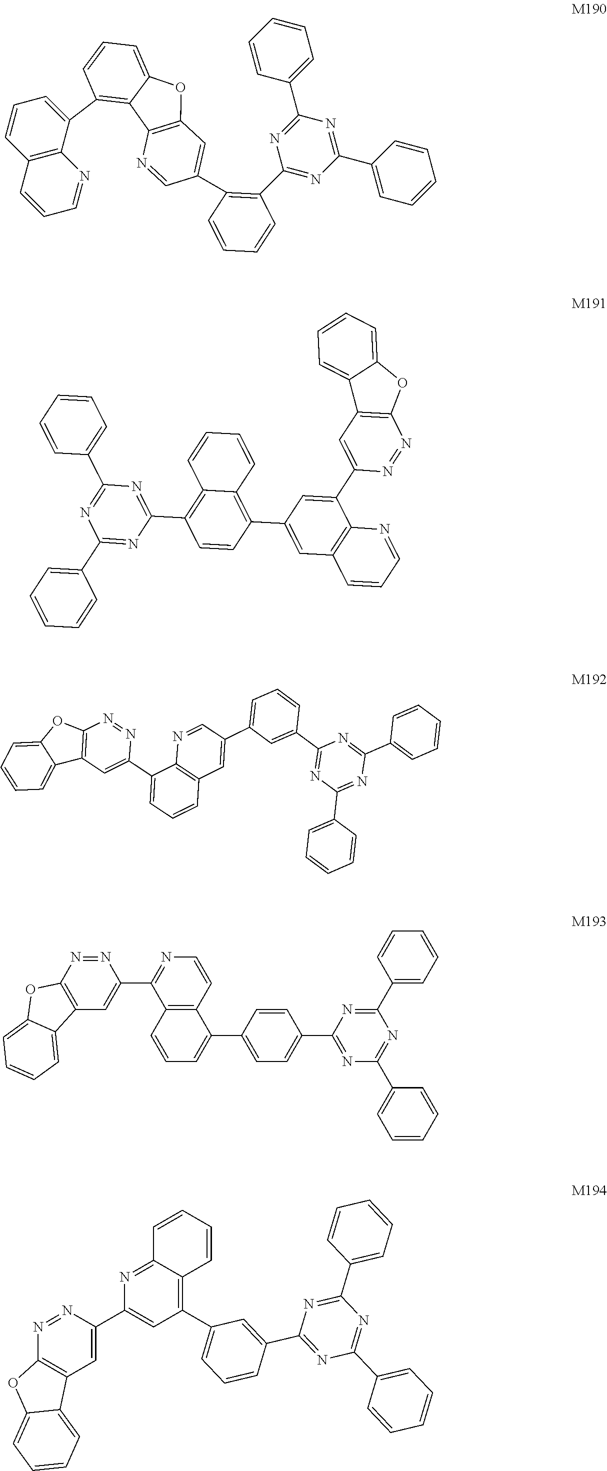 [SMILES: c1ccc(-c2nc(-c3ccccc3)nc(-c3ccccc3-c3cnc4c(c3)oc3cccc(-c5cccc6cccnc56)c34)n2)cc1 ; c1ccc(-c2nc(-c3ccccc3)nc(-c3ccc(-c4cc(-c5cc6c(nn5)oc5ccccc56)c5ncccc5c4)c4ccccc34)n2)cc1 ; c1ccc(-c2nc(-c3ccccc3)nc(-c3cccc(-c4cnc5c(-c6cc7c(nn6)oc6ccccc67)cccc5c4)c3)n2)cc1 ; c1ccc(-c2nc(-c3ccccc3)nc(-c3ccc(-c4cccc5c(-c6cc7c(nn6)oc6ccccc67)nccc45)cc3)n2)cc1 ; c1ccc(-c2nc(-c3ccccc3)nc(-c3cccc(-c4cc(-c5cc6c(nn5)oc5ccccc56)nc5ccccc45)c3)n2)cc1]

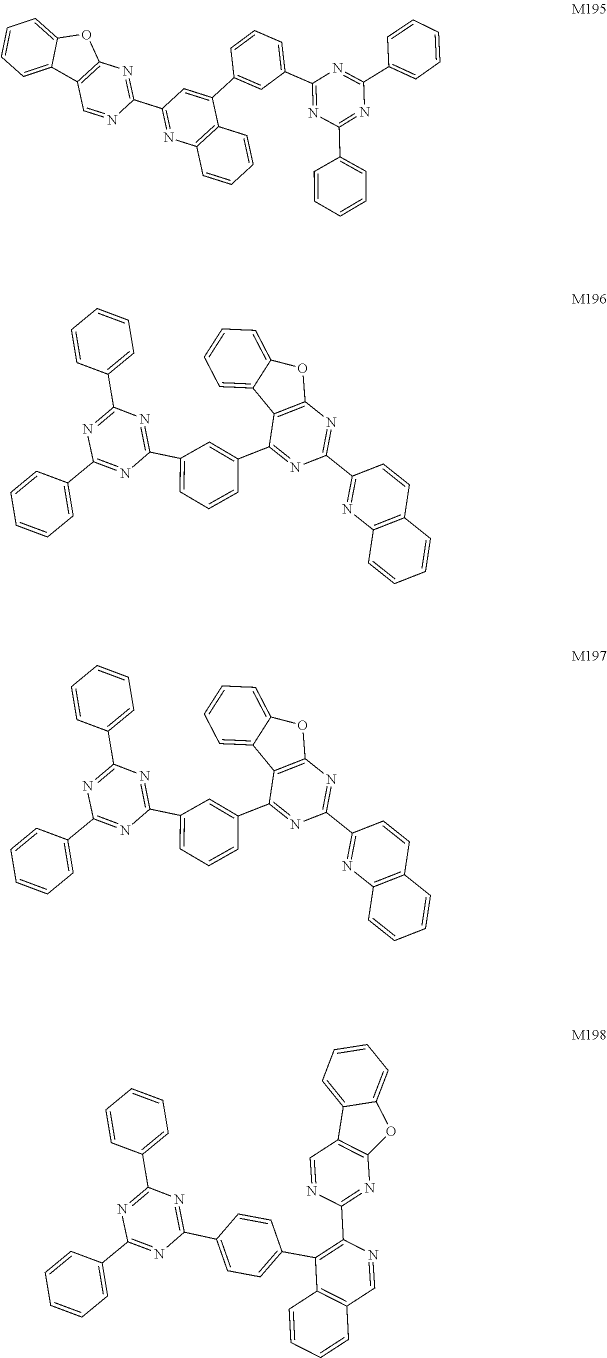 [SMILES: c1ccc(-c2nc(-c3ccccc3)nc(-c3cccc(-c4cc(-c5ncc6c(n5)oc5ccccc56)nc5ccccc45)c3)n2)cc1 ; c1ccc(-c2nc(-c3ccccc3)nc(-c3cccc(-c4nc(-c5ccc6ccccc6n5)nc5oc6ccccc6c45)c3)n2)cc1 ; c1ccc(-c2nc(-c3ccccc3)nc(-c3cccc(-c4nc(-c5ccc6ccccc6n5)nc5oc6ccccc6c45)c3)n2)cc1 ; c1ccc(-c2nc(-c3ccccc3)nc(-c3ccc(-c4c(-c5ncc6c(n5)oc5ccccc56)ncc5ccccc45)cc3)n2)cc1]

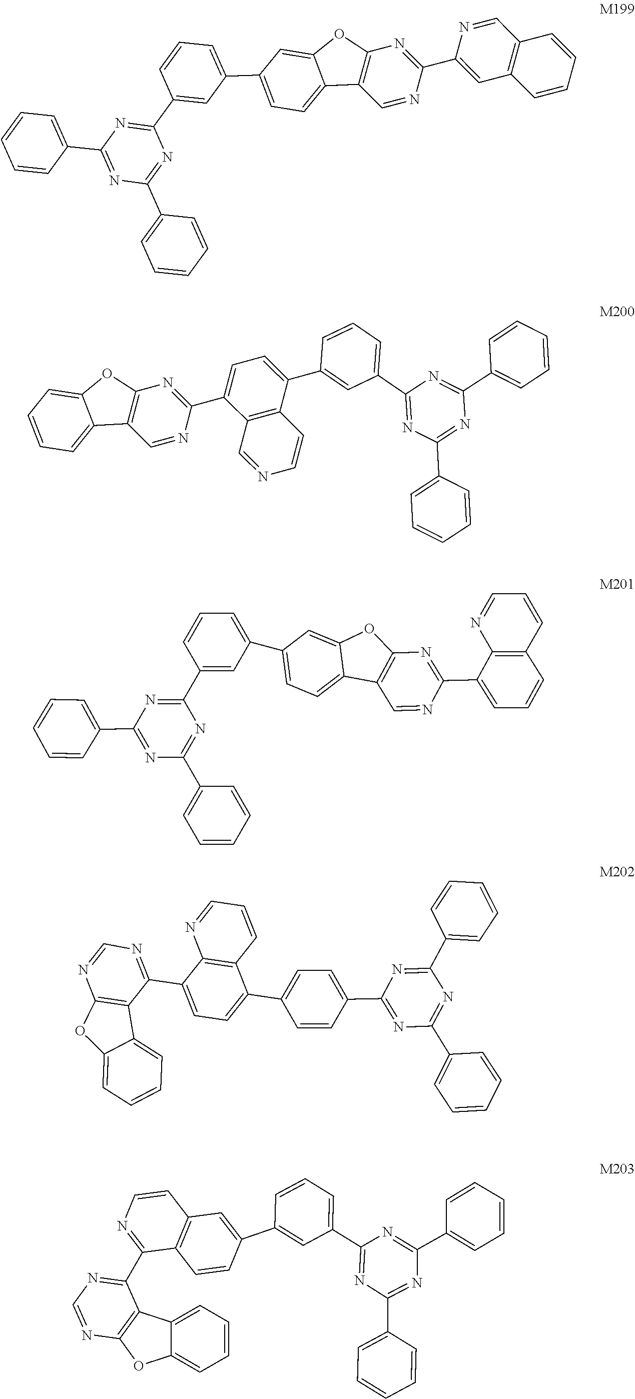 [SMILES: c1ccc(-c2nc(-c3ccccc3)nc(-c3cccc(-c4ccc5c(c4)oc4nc(-c6cc7ccccc7cn6)ncc45)c3)n2)cc1 ; c1ccc(-c2nc(-c3ccccc3)nc(-c3cccc(-c4ccc(-c5ncc6c(n5)oc5ccccc56)c5cnccc45)c3)n2)cc1 ; c1ccc(-c2nc(-c3ccccc3)nc(-c3cccc(-c4ccc5c(c4)oc4nc(-c6cccc7cccnc67)ncc45)c3)n2)cc1 ; c1ccc(-c2nc(-c3ccccc3)nc(-c3ccc(-c4ccc(-c5ncnc6oc7ccccc7c56)c5ncccc45)cc3)n2)cc1 ; c1ccc(-c2nc(-c3ccccc3)nc(-c3cccc(-c4ccc5c(-c6ncnc7oc8ccccc8c67)nccc5c4)c3)n2)cc1]

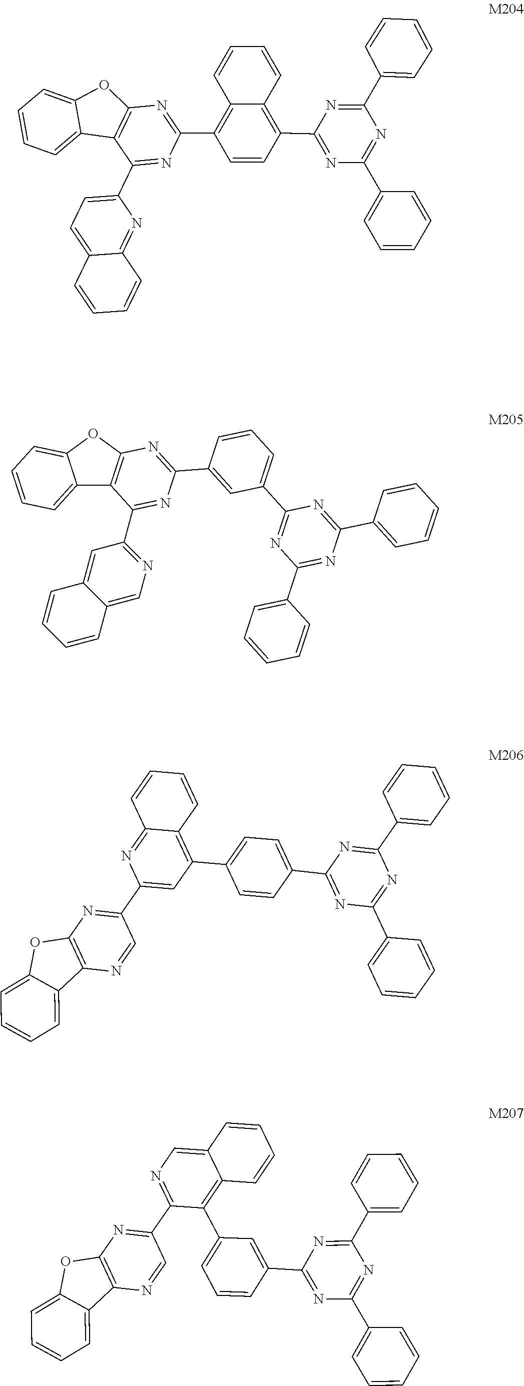 [SMILES: c1ccc(-c2nc(-c3ccccc3)nc(-c3ccc(-c4nc(-c5ccc6ccccc6n5)c5c(n4)oc4ccccc45)c4ccccc34)n2)cc1 ; c1ccc(-c2nc(-c3ccccc3)nc(-c3cccc(-c4nc(-c5cc6ccccc6cn5)c5c(n4)oc4ccccc45)c3)n2)cc1 ; c1ccc(-c2nc(-c3ccccc3)nc(-c3ccc(-c4cc(-c5cnc6c(n5)oc5ccccc56)nc5ccccc45)cc3)n2)cc1 ; c1ccc(-c2nc(-c3ccccc3)nc(-c3cccc(-c4c(-c5cnc6c(n5)oc5ccccc56)ncc5ccccc45)c3)n2)cc1]

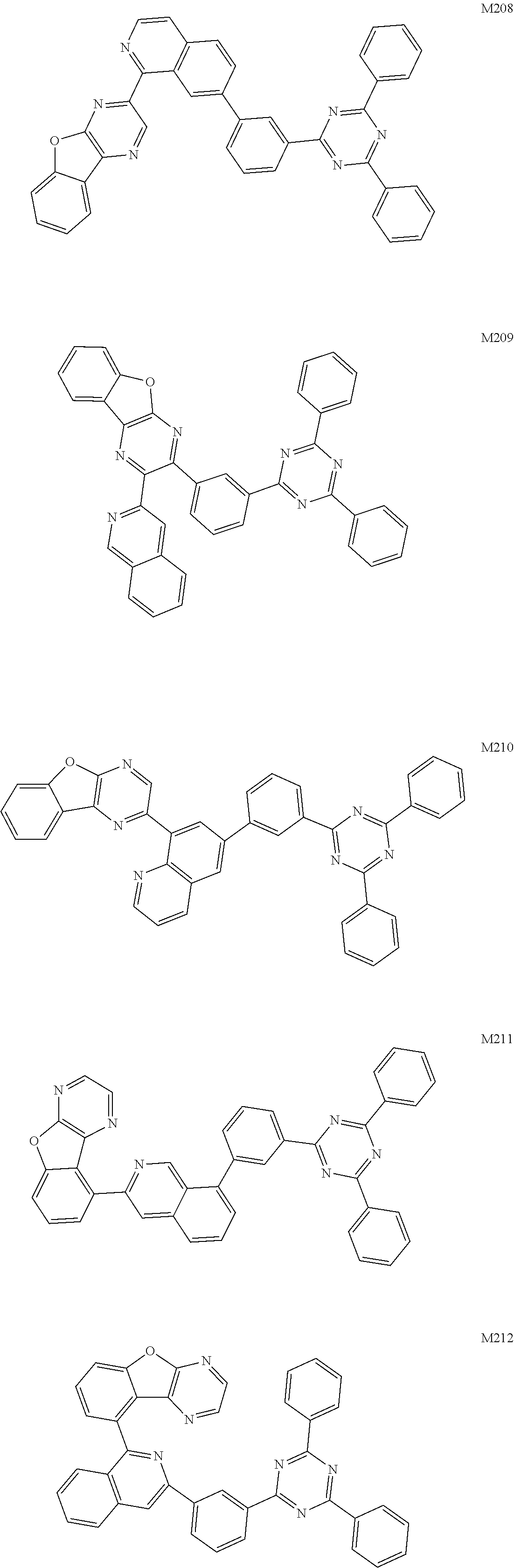 [SMILES: c1ccc(-c2nc(-c3ccccc3)nc(-c3cccc(-c4ccc5ccnc(-c6cnc7c(n6)oc6ccccc67)c5c4)c3)n2)cc1 ; c1ccc(-c2nc(-c3ccccc3)nc(-c3cccc(-c4nc5oc6ccccc6c5nc4-c4cc5ccccc5cn4)c3)n2)cc1 ; c1ccc(-c2nc(-c3ccccc3)nc(-c3cccc(-c4cc(-c5cnc6oc7ccccc7c6n5)c5ncccc5c4)c3)n2)cc1 ; c1ccc(-c2nc(-c3ccccc3)nc(-c3cccc(-c4cccc5cc(-c6cccc7oc8nccnc8c67)ncc45)c3)n2)cc1 ; c1ccc(-c2nc(-c3ccccc3)nc(-c3cccc(-c4cc5ccccc5c(-c5cccc6oc7nccnc7c56)n4)c3)n2)cc1]

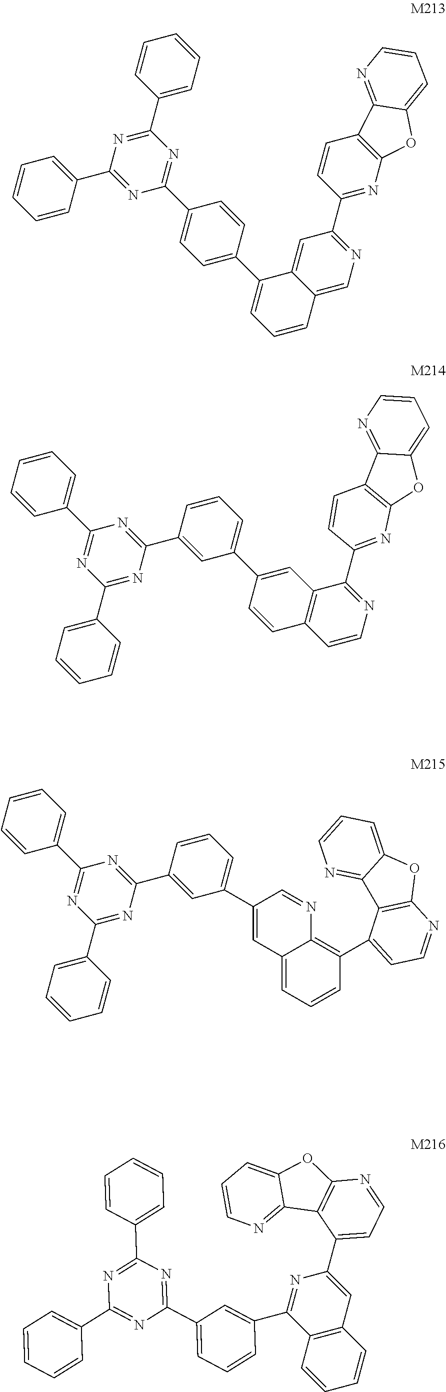 [SMILES: c1ccc(-c2nc(-c3ccccc3)nc(-c3ccc(-c4cccc5cnc(-c6ccc7c(n6)oc6cccnc67)cc45)cc3)n2)cc1 ; c1ccc(-c2nc(-c3ccccc3)nc(-c3cccc(-c4ccc5ccnc(-c6ccc7c(n6)oc6cccnc67)c5c4)c3)n2)cc1 ; c1ccc(-c2nc(-c3ccccc3)nc(-c3cccc(-c4cnc5c(-c6ccnc7oc8cccnc8c67)cccc5c4)c3)n2)cc1 ; c1ccc(-c2nc(-c3ccccc3)nc(-c3cccc(-c4nc(-c5ccnc6oc7cccnc7c56)cc5ccccc45)c3)n2)cc1]

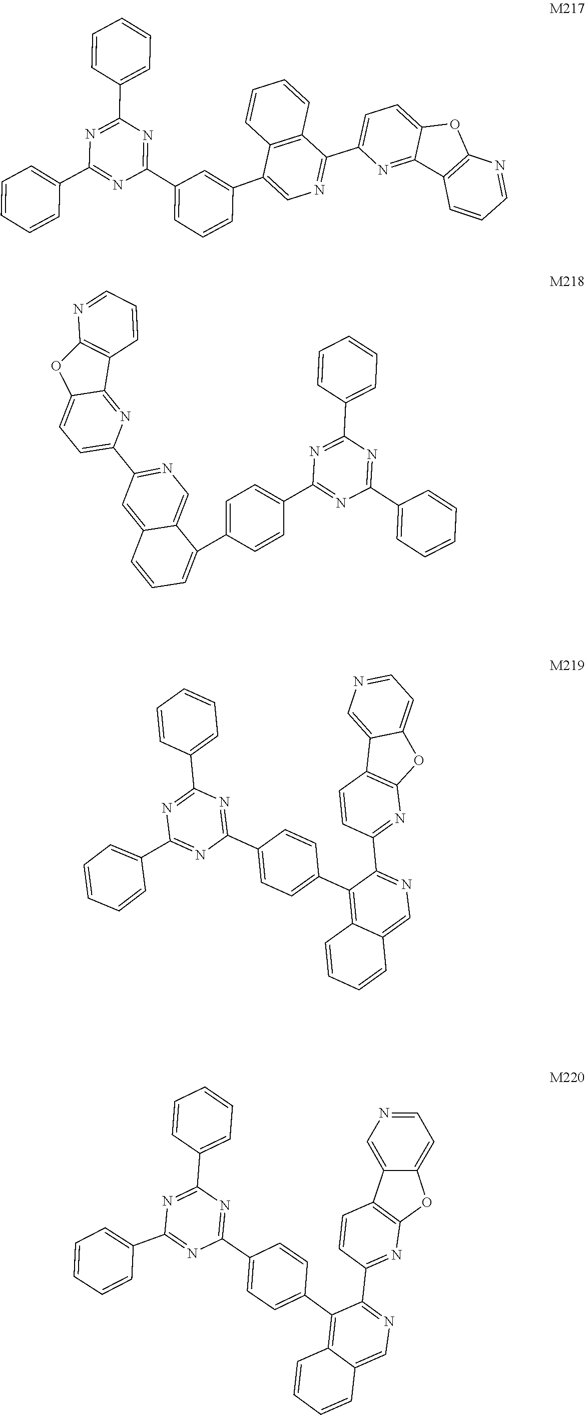 [SMILES: c1ccc(-c2nc(-c3ccccc3)nc(-c3cccc(-c4cnc(-c5ccc6oc7ncccc7c6n5)c5ccccc45)c3)n2)cc1 ; c1ccc(-c2nc(-c3ccccc3)nc(-c3ccc(-c4cccc5cc(-c6ccc7oc8ncccc8c7n6)ncc45)cc3)n2)cc1 ; c1ccc(-c2nc(-c3ccccc3)nc(-c3ccc(-c4c(-c5ccc6c(n5)oc5ccncc56)ncc5ccccc45)cc3)n2)cc1 ; c1ccc(-c2nc(-c3ccccc3)nc(-c3ccc(-c4c(-c5ccc6c(n5)oc5ccncc56)ncc5ccccc45)cc3)n2)cc1]

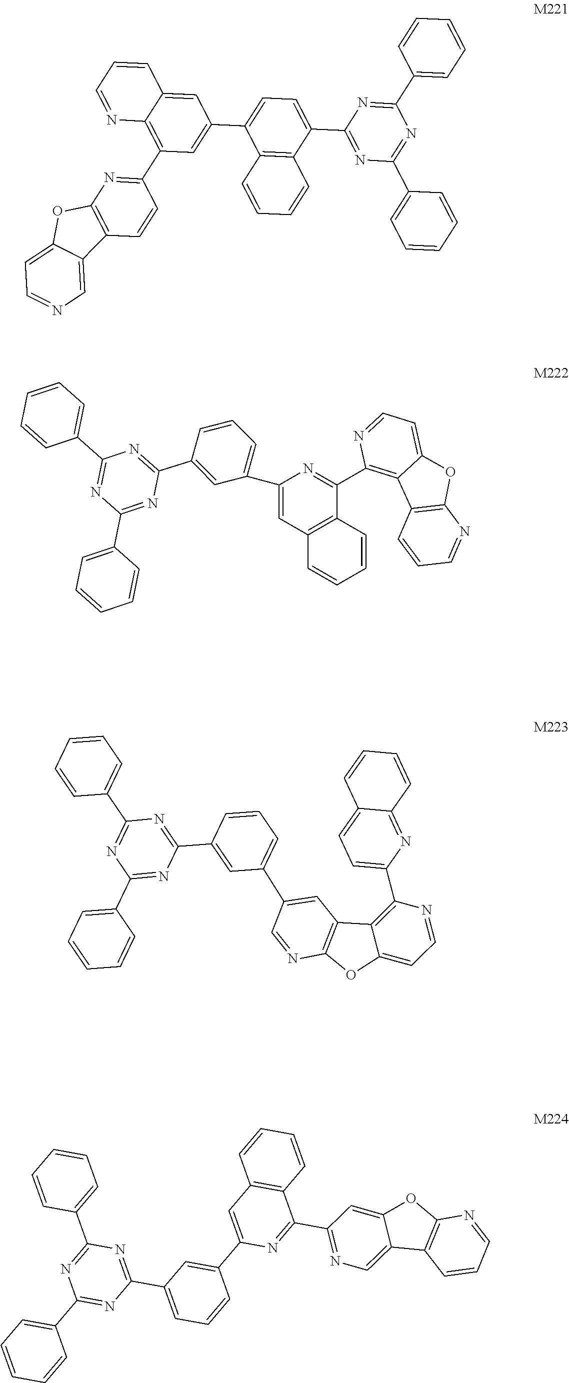 [SMILES: c1ccc(-c2nc(-c3ccccc3)nc(-c3ccc(-c4cc(-c5ccc6c(n5)oc5ccncc56)c5ncccc5c4)c4ccccc34)n2)cc1 ; c1ccc(-c2nc(-c3ccccc3)nc(-c3cccc(-c4cc5ccccc5c(-c5nccc6oc7ncccc7c56)n4)c3)n2)cc1 ; c1ccc(-c2nc(-c3ccccc3)nc(-c3cccc(-c4cnc5oc6ccnc(-c7ccc8ccccc8n7)c6c5c4)c3)n2)cc1 ; c1ccc(-c2nc(-c3ccccc3)nc(-c3cccc(-c4cc5ccccc5c(-c5cc6oc7ncccc7c6cn5)n4)c3)n2)cc1]

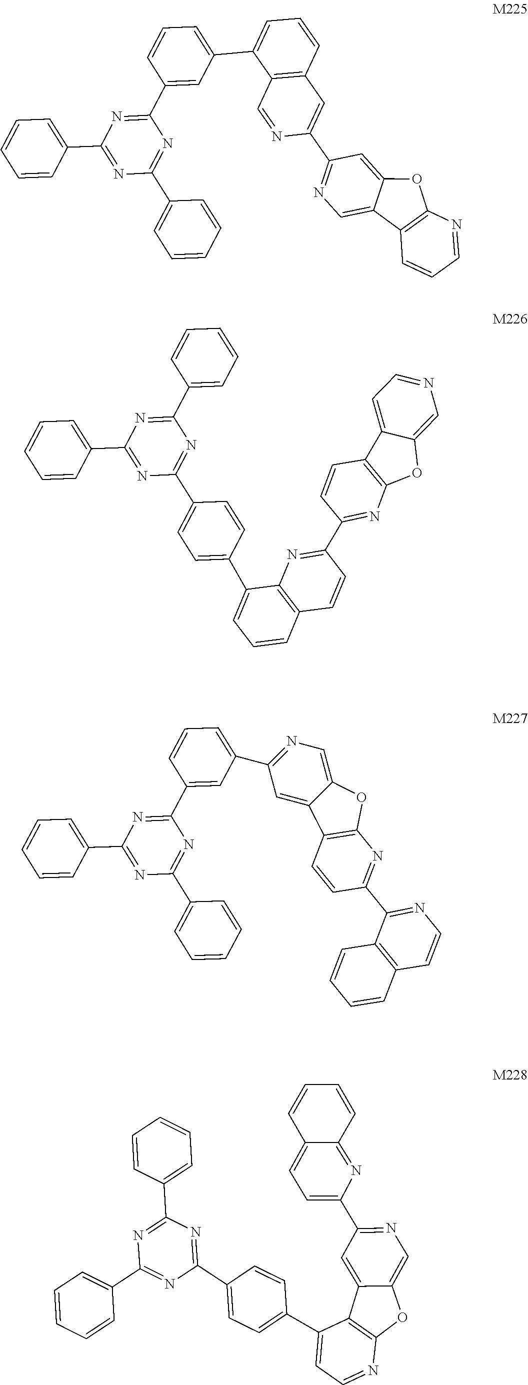 [SMILES: c1ccc(-c2nc(-c3ccccc3)nc(-c3cccc(-c4cccc5cc(-c6cc7oc8ncccc8c7cn6)ncc45)c3)n2)cc1 ; c1ccc(-c2nc(-c3ccccc3)nc(-c3ccc(-c4cccc5ccc(-c6ccc7c(n6)oc6cnccc67)nc45)cc3)n2)cc1 ; c1ccc(-c2nc(-c3ccccc3)nc(-c3cccc(-c4cc5c(cn4)oc4nc(-c6nccc7ccccc67)ccc45)c3)n2)cc1 ; c1ccc(-c2nc(-c3ccccc3)nc(-c3ccc(-c4ccnc5oc6cnc(-c7ccc8ccccc8n7)cc6c45)cc3)n2)cc1]

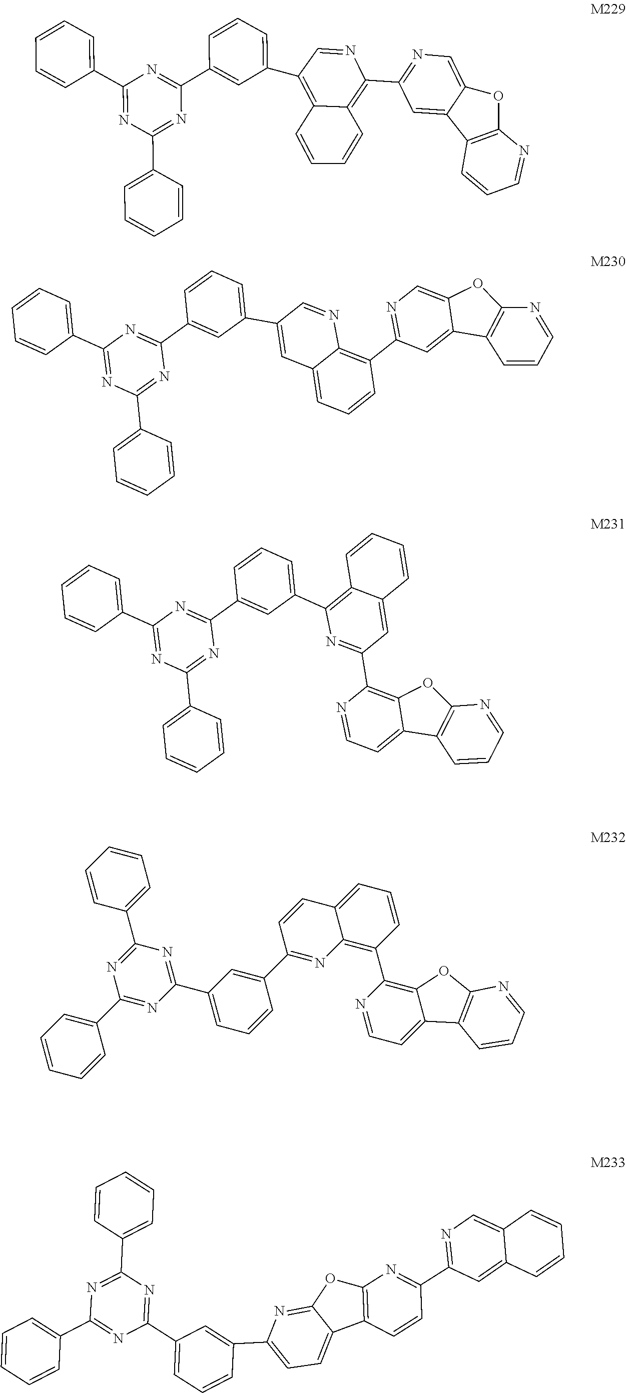 [SMILES: c1ccc(-c2nc(-c3ccccc3)nc(-c3cccc(-c4cnc(-c5cc6c(cn5)oc5ncccc56)c5ccccc45)c3)n2)cc1 ; c1ccc(-c2nc(-c3ccccc3)nc(-c3cccc(-c4cnc5c(-c6cc7c(cn6)oc6ncccc67)cccc5c4)c3)n2)cc1 ; c1ccc(-c2nc(-c3ccccc3)nc(-c3cccc(-c4nc(-c5nccc6c5oc5ncccc56)cc5ccccc45)c3)n2)cc1 ; c1ccc(-c2nc(-c3ccccc3)nc(-c3cccc(-c4ccc5cccc(-c6nccc7c6oc6ncccc67)c5n4)c3)n2)cc1 ; c1ccc(-c2nc(-c3ccccc3)nc(-c3cccc(-c4ccc5c(n4)oc4nc(-c6cc7ccccc7cn6)ccc45)c3)n2)cc1]

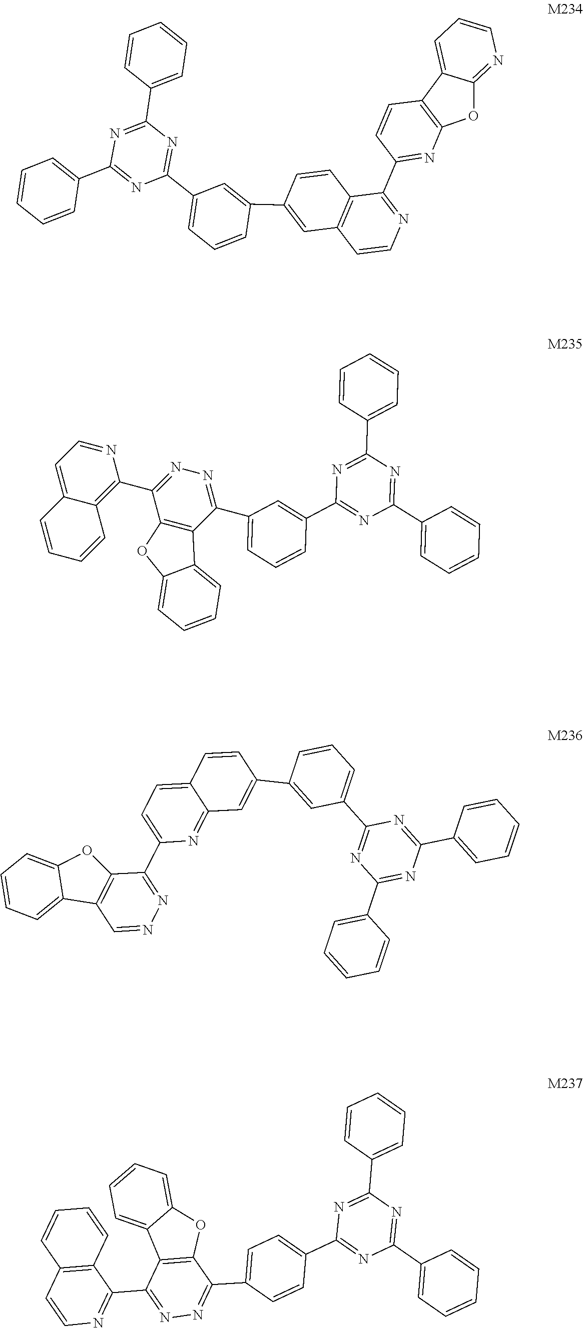 [SMILES: c1ccc(-c2nc(-c3ccccc3)nc(-c3cccc(-c4ccc5c(-c6ccc7c(n6)oc6ncccc67)nccc5c4)c3)n2)cc1 ; c1ccc(-c2nc(-c3ccccc3)nc(-c3cccc(-c4nnc(-c5nccc6ccccc56)c5oc6ccccc6c45)c3)n2)cc1 ; c1ccc(-c2nc(-c3ccccc3)nc(-c3cccc(-c4ccc5ccc(-c6nncc7c6oc6ccccc67)nc5c4)c3)n2)cc1 ; c1ccc(-c2nc(-c3ccccc3)nc(-c3ccc(-c4nnc(-c5nccc6ccccc56)c5c4oc4ccccc45)cc3)n2)cc1]

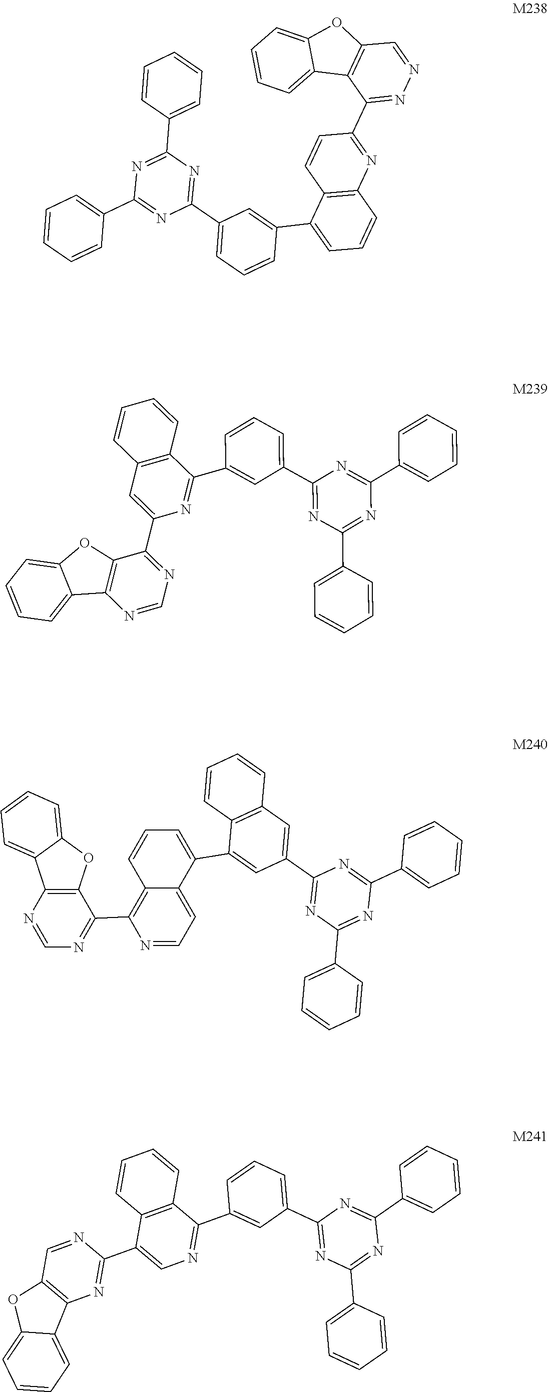 [SMILES: c1ccc(-c2nc(-c3ccccc3)nc(-c3cccc(-c4cccc5nc(-c6nncc7oc8ccccc8c67)ccc45)c3)n2)cc1 ; c1ccc(-c2nc(-c3ccccc3)nc(-c3cccc(-c4nc(-c5ncnc6c5oc5ccccc56)cc5ccccc45)c3)n2)cc1 ; c1ccc(-c2nc(-c3ccccc3)nc(-c3cc(-c4cccc5c(-c6ncnc7c6oc6ccccc67)nccc45)c4ccccc4c3)n2)cc1 ; c1ccc(-c2nc(-c3ccccc3)nc(-c3cccc(-c4ncc(-c5ncc6oc7ccccc7c6n5)c5ccccc45)c3)n2)cc1]

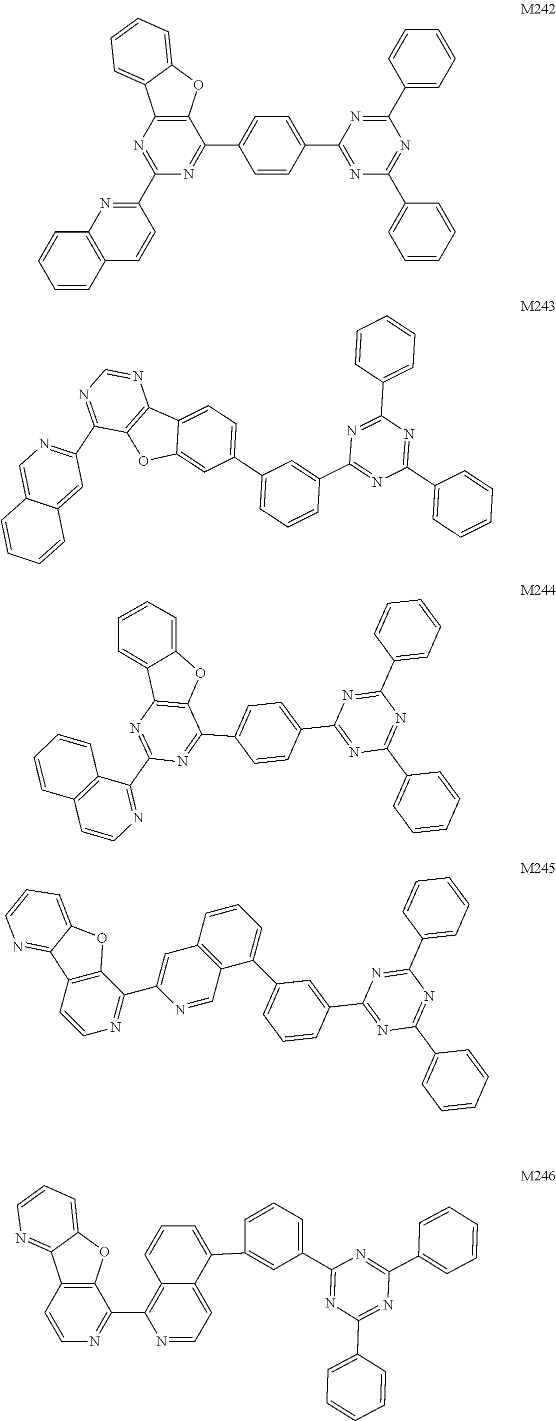 [SMILES: c1ccc(-c2nc(-c3ccccc3)nc(-c3ccc(-c4nc(-c5ccc6ccccc6n5)nc5c4oc4ccccc45)cc3)n2)cc1 ; c1ccc(-c2nc(-c3ccccc3)nc(-c3cccc(-c4ccc5c(c4)oc4c(-c6cc7ccccc7cn6)ncnc45)c3)n2)cc1 ; c1ccc(-c2nc(-c3ccccc3)nc(-c3ccc(-c4nc(-c5nccc6ccccc56)nc5c4oc4ccccc45)cc3)n2)cc1 ; c1ccc(-c2nc(-c3ccccc3)nc(-c3cccc(-c4cccc5cc(-c6nccc7c6oc6cccnc67)ncc45)c3)n2)cc1 ; c1ccc(-c2nc(-c3ccccc3)nc(-c3cccc(-c4cccc5c(-c6nccc7c6oc6cccnc67)nccc45)c3)n2)cc1]

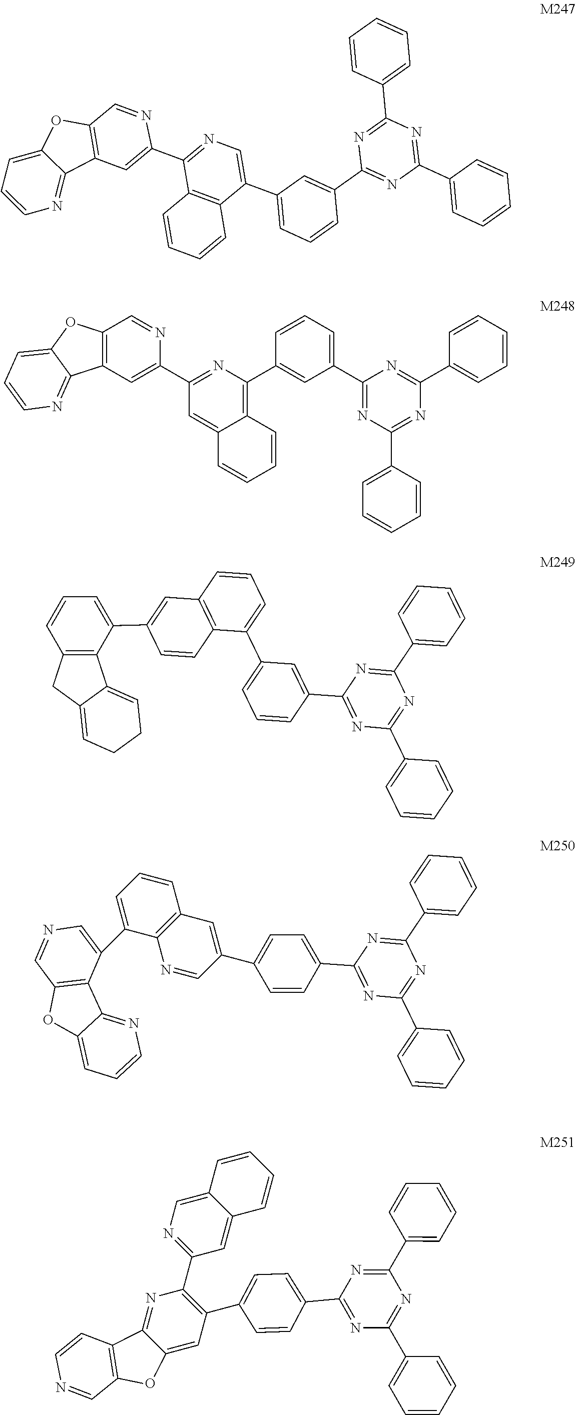

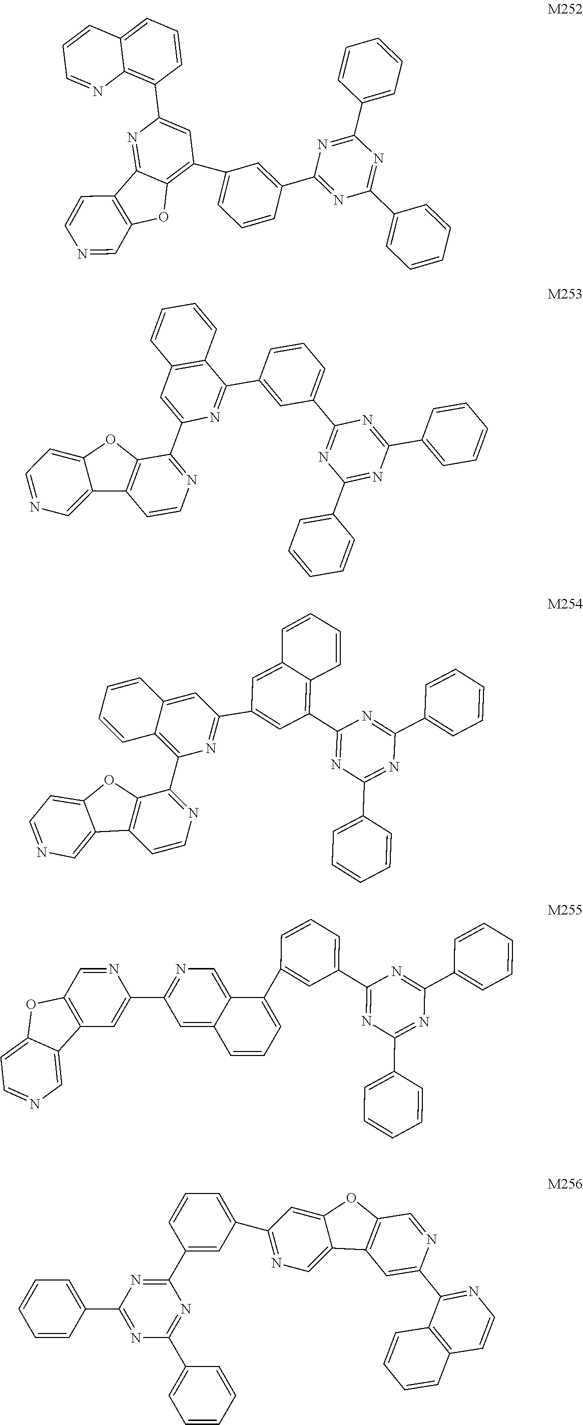

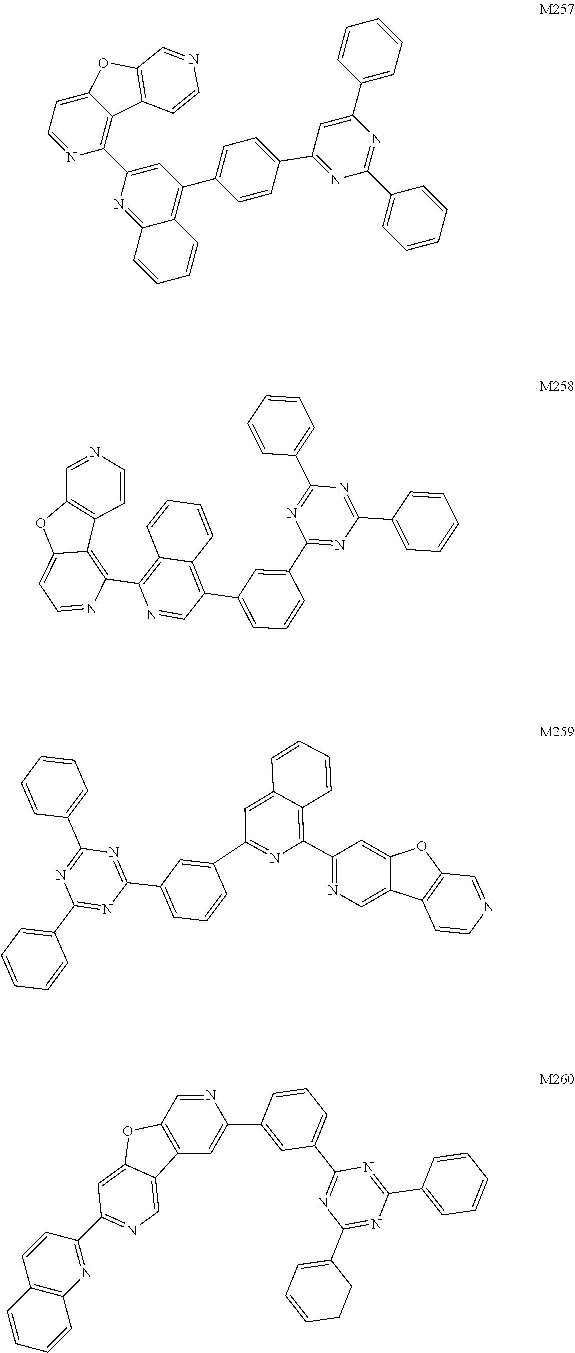 [SMILES: c1ccc(-c2cc(-c3ccc(-c4cc(-c5nccc6oc7cnccc7c56)nc5ccccc45)cc3)nc(-c3ccccc3)n2)cc1 ; c1ccc(-c2nc(-c3ccccc3)nc(-c3cccc(-c4cnc(-c5nccc6oc7cnccc7c56)c5ccccc45)c3)n2)cc1 ; c1ccc(-c2nc(-c3ccccc3)nc(-c3cccc(-c4cc5ccccc5c(-c5cc6oc7cnccc7c6cn5)n4)c3)n2)cc1 ; C1=CCCC(c2nc(-c3ccccc3)nc(-c3cccc(-c4cc5c(cn4)oc4cc(-c6ccc7ccccc7n6)ncc45)c3)n2)=C1]

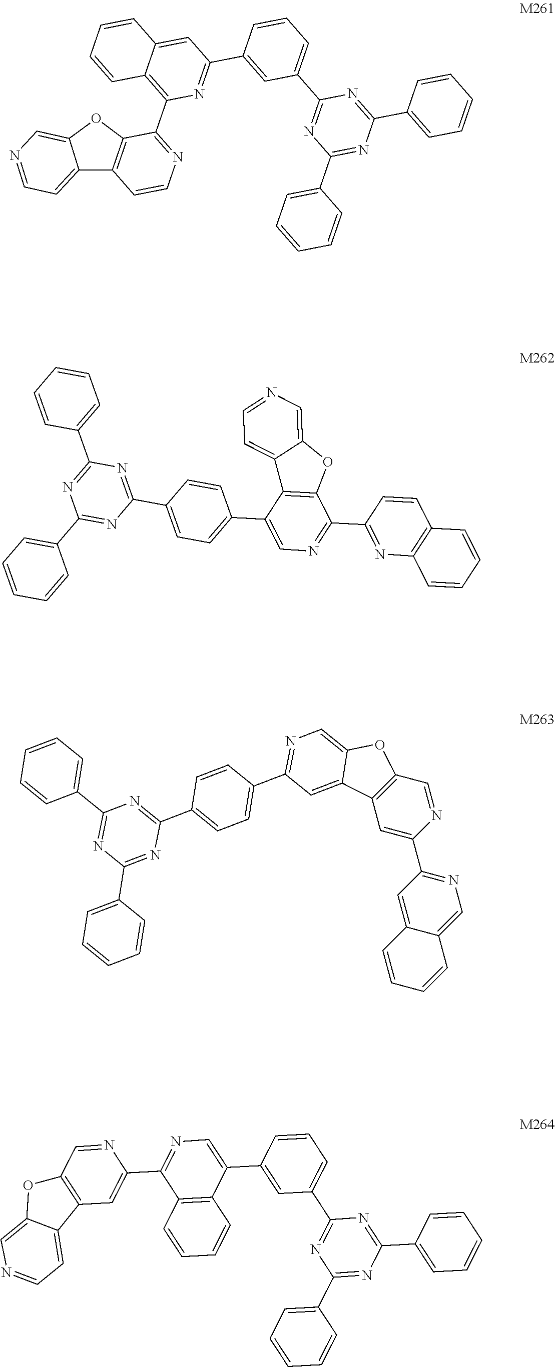 [SMILES: c1ccc(-c2nc(-c3ccccc3)nc(-c3cccc(-c4cc5ccccc5c(-c5nccc6c5oc5cnccc56)n4)c3)n2)cc1 ; c1ccc(-c2nc(-c3ccccc3)nc(-c3ccc(-c4cnc(-c5ccc6ccccc6n5)c5oc6cnccc6c45)cc3)n2)cc1 ; c1ccc(-c2nc(-c3ccccc3)nc(-c3ccc(-c4cc5c(cn4)oc4cnc(-c6cc7ccccc7cn6)cc45)cc3)n2)cc1 ; c1ccc(-c2nc(-c3ccccc3)nc(-c3cccc(-c4cnc(-c5cc6c(cn5)oc5cnccc56)c5ccccc45)c3)n2)cc1]

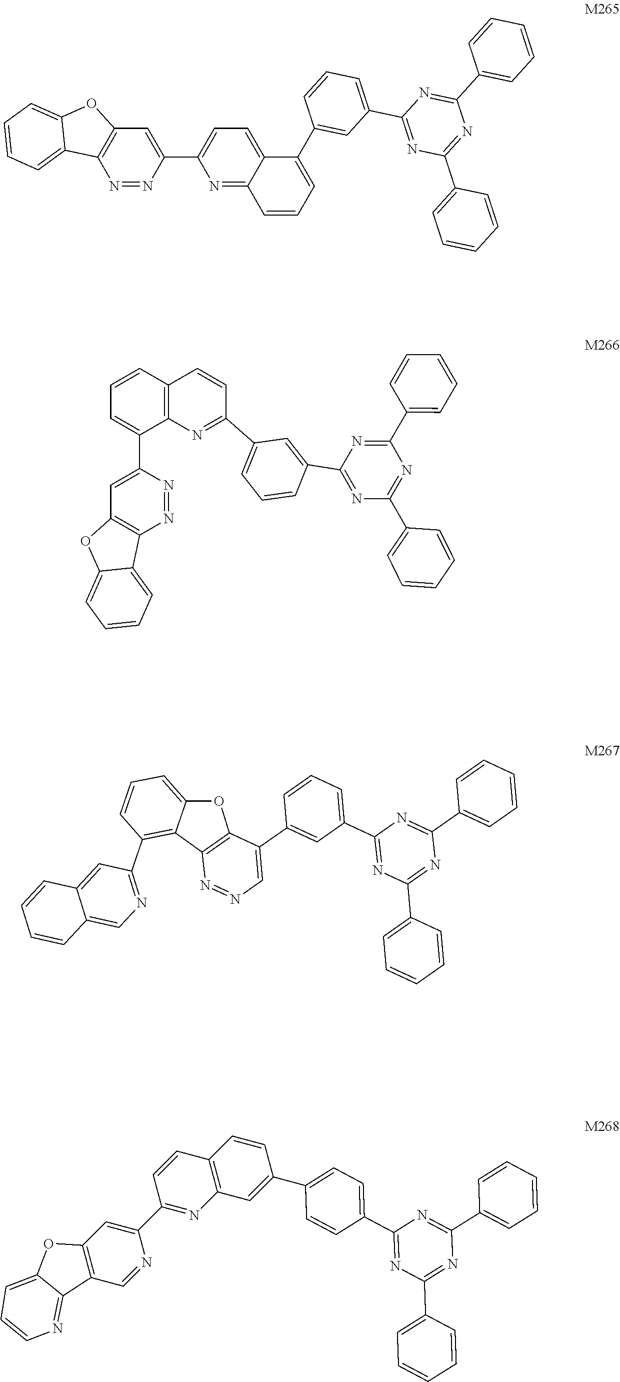 [SMILES: c1ccc(-c2nc(-c3ccccc3)nc(-c3cccc(-c4cccc5nc(-c6cc7oc8ccccc8c7nn6)ccc45)c3)n2)cc1 ; c1ccc(-c2nc(-c3ccccc3)nc(-c3cccc(-c4ccc5cccc(-c6cc7oc8ccccc8c7nn6)c5n4)c3)n2)cc1 ; c1ccc(-c2nc(-c3ccccc3)nc(-c3cccc(-c4cnnc5c4oc4cccc(-c6cc7ccccc7cn6)c45)c3)n2)cc1 ; c1ccc(-c2nc(-c3ccccc3)nc(-c3ccc(-c4ccc5ccc(-c6cc7oc8cccnc8c7cn6)nc5c4)cc3)n2)cc1]

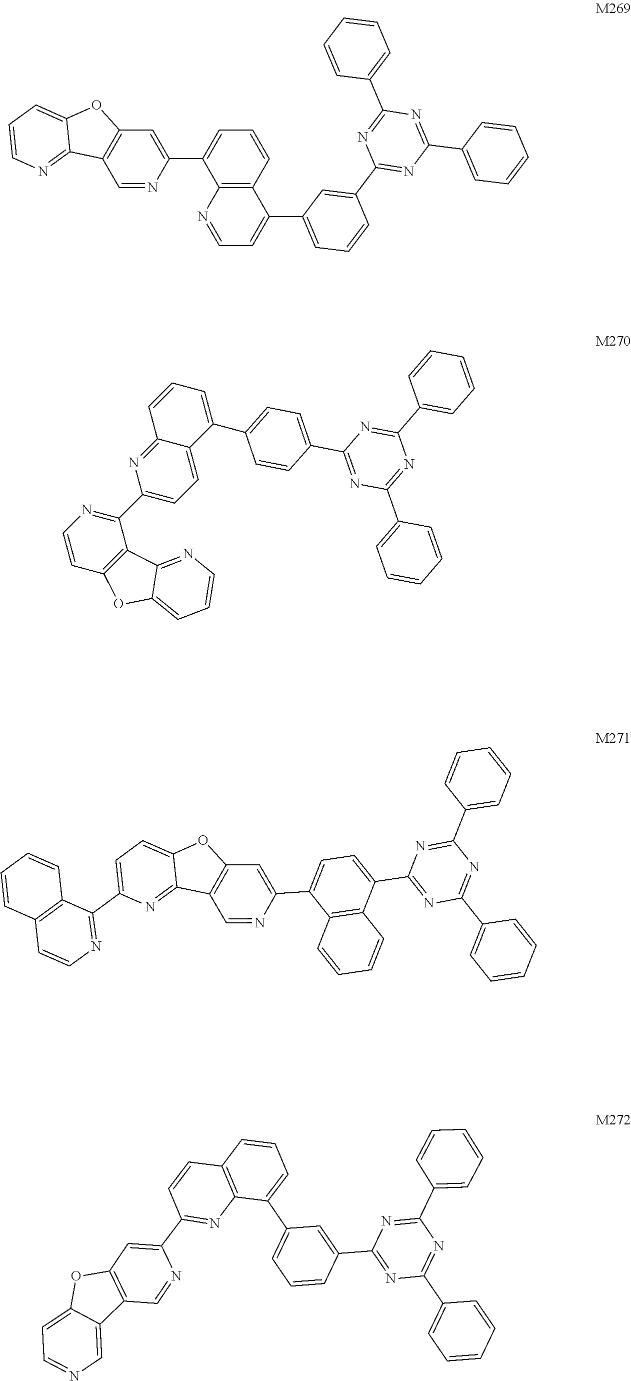 [SMILES: c1ccc(-c2nc(-c3ccccc3)nc(-c3cccc(-c4ccnc5c(-c6cc7oc8cccnc8c7cn6)cccc45)c3)n2)cc1 ; c1ccc(-c2nc(-c3ccccc3)nc(-c3ccc(-c4cccc5nc(-c6nccc7oc8cccnc8c67)ccc45)cc3)n2)cc1 ; c1ccc(-c2nc(-c3ccccc3)nc(-c3ccc(-c4cc5oc6ccc(-c7nccc8ccccc78)nc6c5cn4)c4ccccc34)n2)cc1 ; c1ccc(-c2nc(-c3ccccc3)nc(-c3cccc(-c4cccc5ccc(-c6cc7oc8ccncc8c7cn6)nc45)c3)n2)cc1]

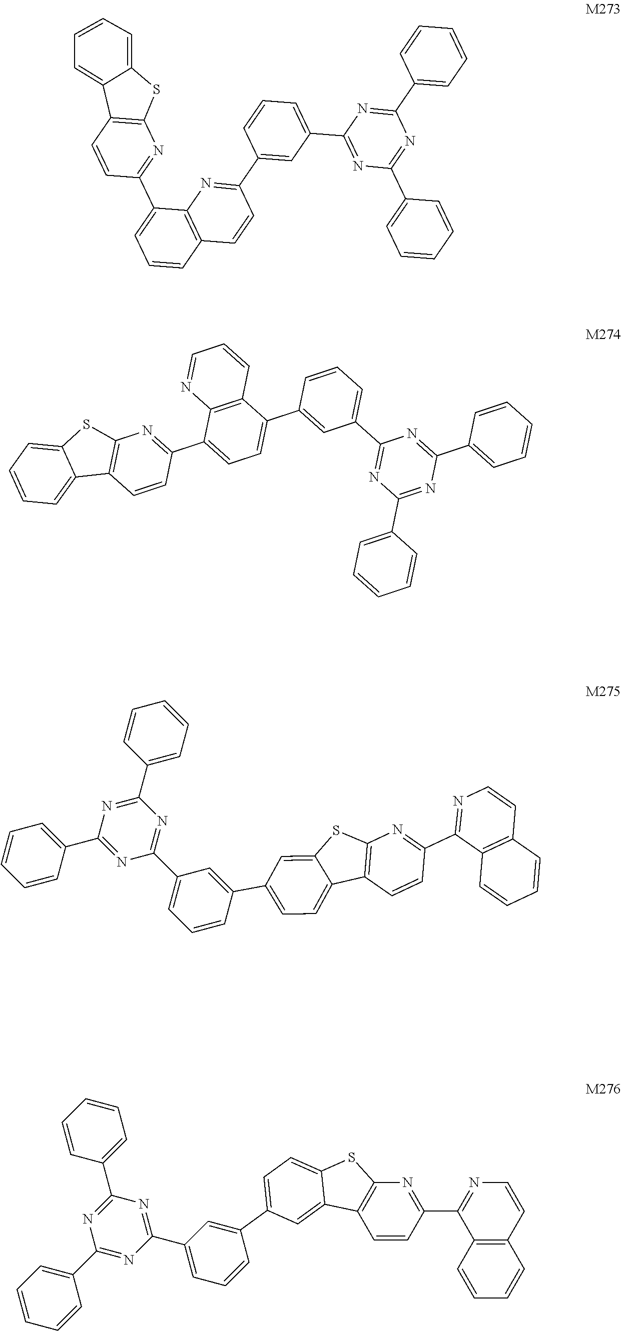 [SMILES: c1ccc(-c2nc(-c3ccccc3)nc(-c3cccc(-c4ccc5cccc(-c6ccc7c(n6)sc6ccccc67)c5n4)c3)n2)cc1 ; c1ccc(-c2nc(-c3ccccc3)nc(-c3cccc(-c4ccc(-c5ccc6c(n5)sc5ccccc56)c5ncccc45)c3)n2)cc1 ; c1ccc(-c2nc(-c3ccccc3)nc(-c3cccc(-c4ccc5c(c4)sc4nc(-c6nccc7ccccc67)ccc45)c3)n2)cc1 ; c1ccc(-c2nc(-c3ccccc3)nc(-c3cccc(-c4ccc5sc6nc(-c7nccc8ccccc78)ccc6c5c4)c3)n2)cc1]

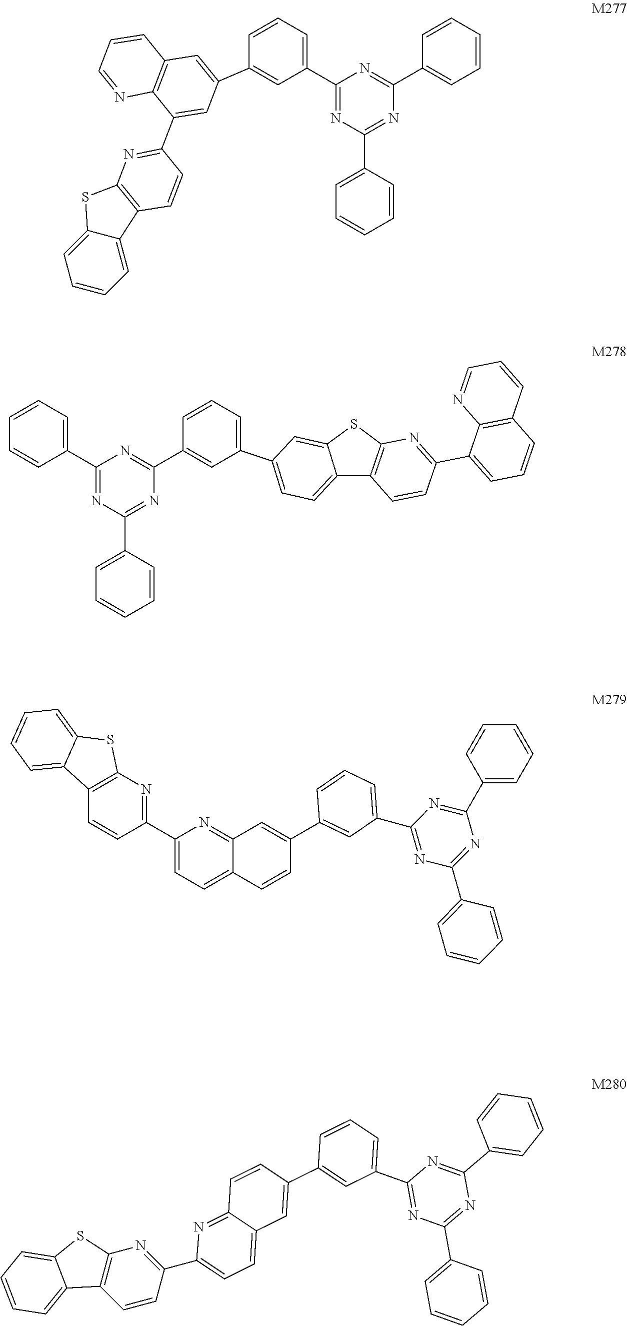 [SMILES: c1ccc(-c2nc(-c3ccccc3)nc(-c3cccc(-c4cc(-c5ccc6c(n5)sc5ccccc56)c5ncccc5c4)c3)n2)cc1 ; c1ccc(-c2nc(-c3ccccc3)nc(-c3cccc(-c4ccc5c(c4)sc4nc(-c6cccc7cccnc67)ccc45)c3)n2)cc1 ; c1ccc(-c2nc(-c3ccccc3)nc(-c3cccc(-c4ccc5ccc(-c6ccc7c(n6)sc6ccccc67)nc5c4)c3)n2)cc1 ; c1ccc(-c2nc(-c3ccccc3)nc(-c3cccc(-c4ccc5nc(-c6ccc7c(n6)sc6ccccc67)ccc5c4)c3)n2)cc1]

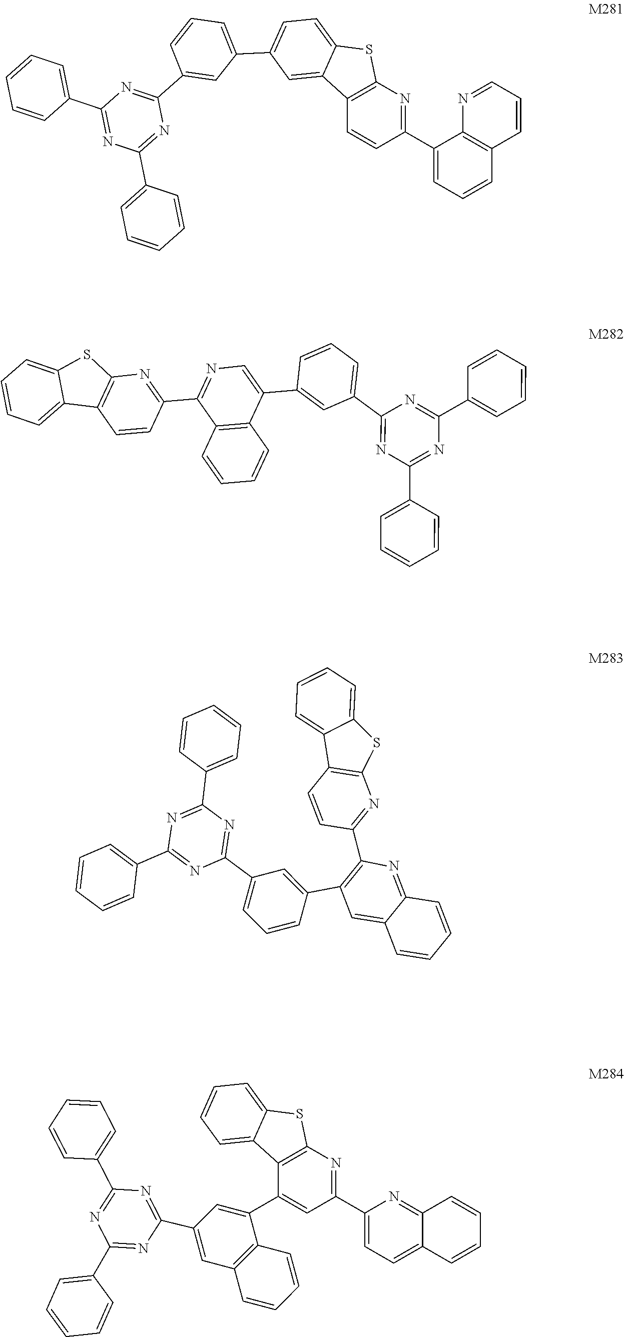 [SMILES: c1ccc(-c2nc(-c3ccccc3)nc(-c3cccc(-c4ccc5sc6nc(-c7cccc8cccnc78)ccc6c5c4)c3)n2)cc1 ; c1ccc(-c2nc(-c3ccccc3)nc(-c3cccc(-c4cnc(-c5ccc6c(n5)sc5ccccc56)c5ccccc45)c3)n2)cc1 ; c1ccc(-c2nc(-c3ccccc3)nc(-c3cccc(-c4cc5ccccc5nc4-c4ccc5c(n4)sc4ccccc45)c3)n2)cc1 ; c1ccc(-c2nc(-c3ccccc3)nc(-c3cc(-c4cc(-c5ccc6ccccc6n5)nc5sc6ccccc6c45)c4ccccc4c3)n2)cc1]

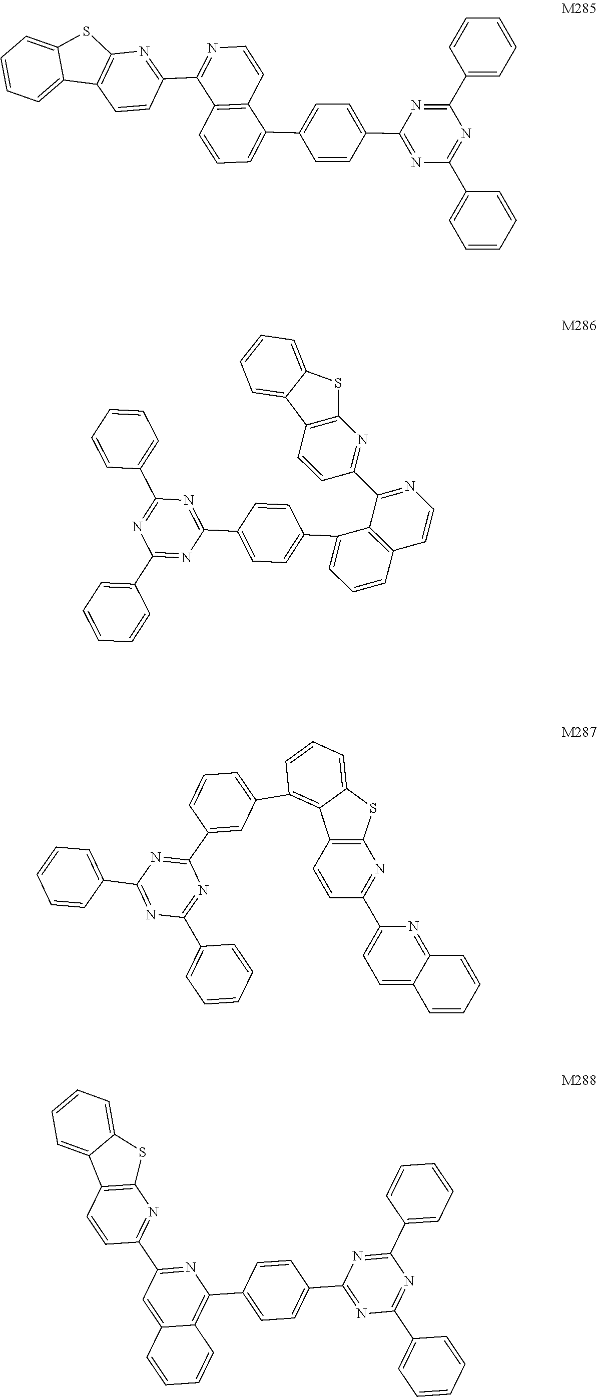 [SMILES: c1ccc(-c2nc(-c3ccccc3)nc(-c3ccc(-c4cccc5c(-c6ccc7c(n6)sc6ccccc67)nccc45)cc3)n2)cc1 ; c1ccc(-c2nc(-c3ccccc3)nc(-c3ccc(-c4cccc5ccnc(-c6ccc7c(n6)sc6ccccc67)c45)cc3)n2)cc1 ; c1ccc(-c2nc(-c3ccccc3)nc(-c3cccc(-c4cccc5sc6nc(-c7ccc8ccccc8n7)ccc6c45)c3)n2)cc1 ; c1ccc(-c2nc(-c3ccccc3)nc(-c3ccc(-c4nc(-c5ccc6c(n5)sc5ccccc56)cc5ccccc45)cc3)n2)cc1]

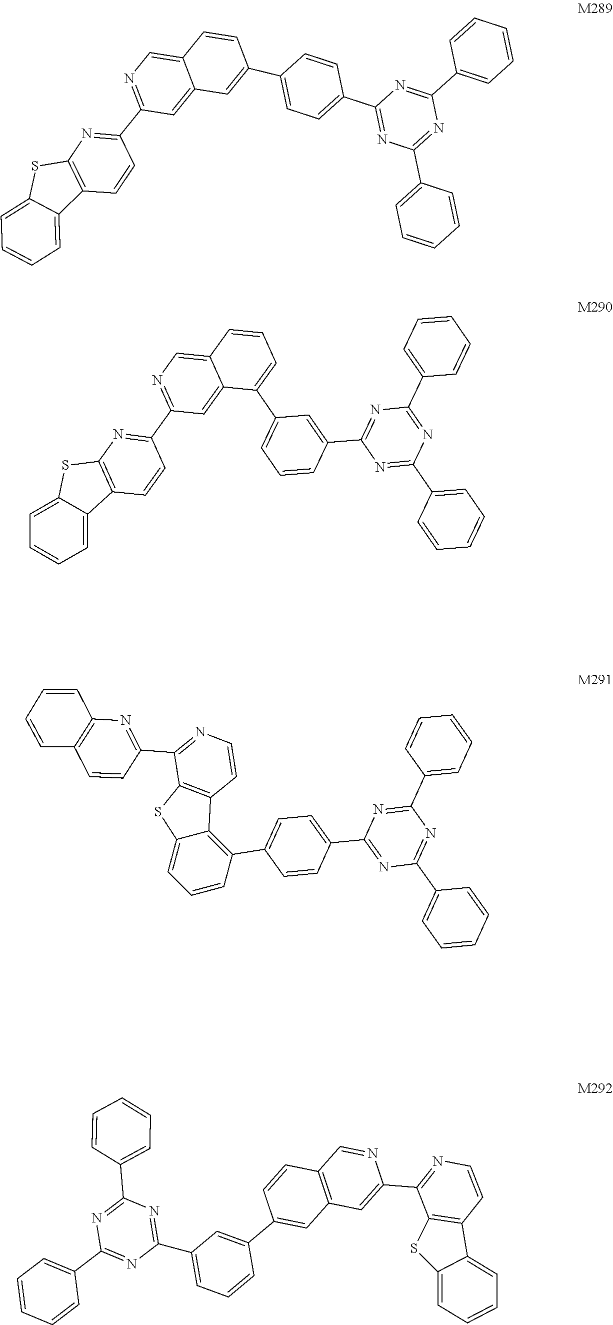 [SMILES: c1ccc(-c2nc(-c3ccccc3)nc(-c3ccc(-c4ccc5cnc(-c6ccc7c(n6)sc6ccccc67)cc5c4)cc3)n2)cc1 ; c1ccc(-c2nc(-c3ccccc3)nc(-c3cccc(-c4cccc5cnc(-c6ccc7c(n6)sc6ccccc67)cc45)c3)n2)cc1 ; c1ccc(-c2nc(-c3ccccc3)nc(-c3ccc(-c4cccc5sc6c(-c7ccc8ccccc8n7)nccc6c45)cc3)n2)cc1 ; c1ccc(-c2nc(-c3ccccc3)nc(-c3cccc(-c4ccc5cnc(-c6nccc7c6sc6ccccc67)cc5c4)c3)n2)cc1]

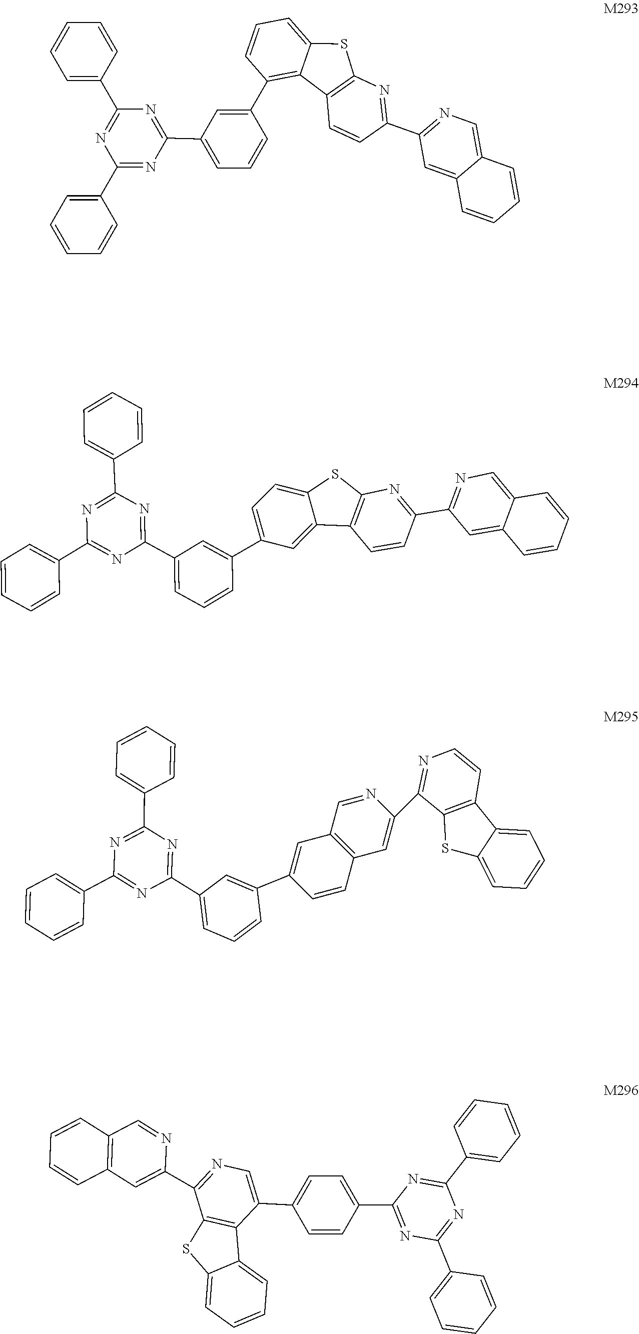 [SMILES: c1ccc(-c2nc(-c3ccccc3)nc(-c3cccc(-c4cccc5sc6nc(-c7cc8ccccc8cn7)ccc6c45)c3)n2)cc1 ; c1ccc(-c2nc(-c3ccccc3)nc(-c3cccc(-c4ccc5sc6nc(-c7cc8ccccc8cn7)ccc6c5c4)c3)n2)cc1 ; c1ccc(-c2nc(-c3ccccc3)nc(-c3cccc(-c4ccc5cc(-c6nccc7c6sc6ccccc67)ncc5c4)c3)n2)cc1 ; c1ccc(-c2nc(-c3ccccc3)nc(-c3ccc(-c4cnc(-c5cc6ccccc6cn5)c5sc6ccccc6c45)cc3)n2)cc1]

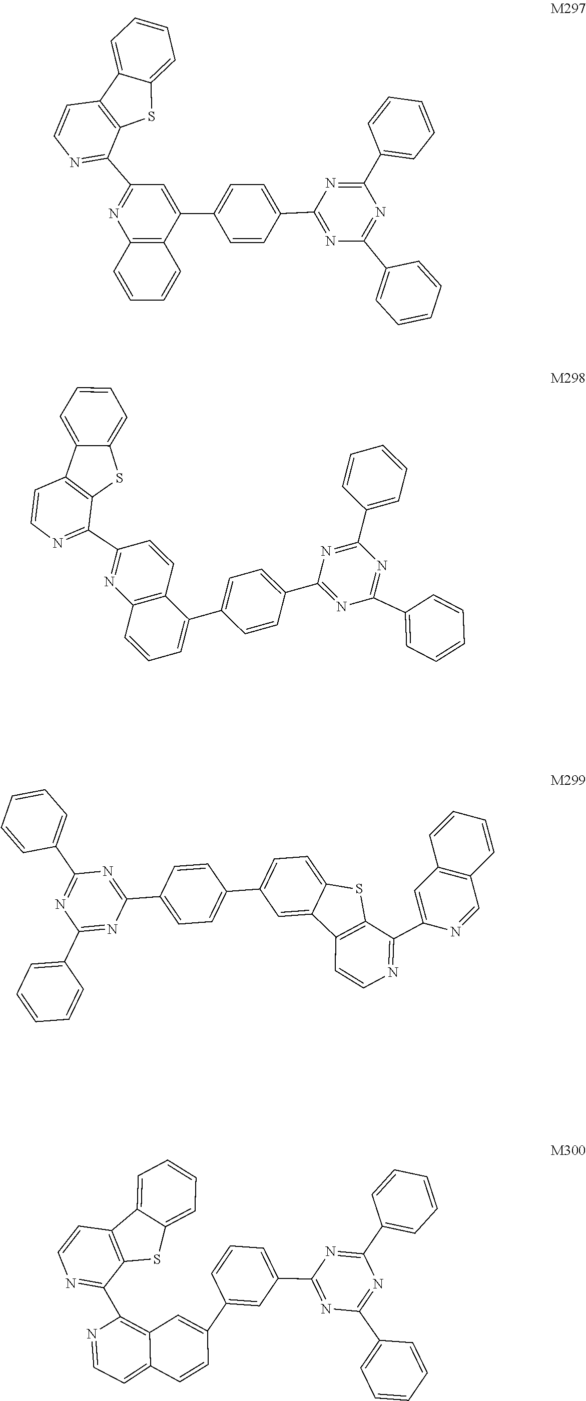 [SMILES: c1ccc(-c2nc(-c3ccccc3)nc(-c3ccc(-c4cc(-c5nccc6c5sc5ccccc56)nc5ccccc45)cc3)n2)cc1 ; c1ccc(-c2nc(-c3ccccc3)nc(-c3ccc(-c4cccc5nc(-c6nccc7c6sc6ccccc67)ccc45)cc3)n2)cc1 ; c1ccc(-c2nc(-c3ccccc3)nc(-c3ccc(-c4ccc5sc6c(-c7cc8ccccc8cn7)nccc6c5c4)cc3)n2)cc1 ; c1ccc(-c2nc(-c3ccccc3)nc(-c3cccc(-c4ccc5ccnc(-c6nccc7c6sc6ccccc67)c5c4)c3)n2)cc1]

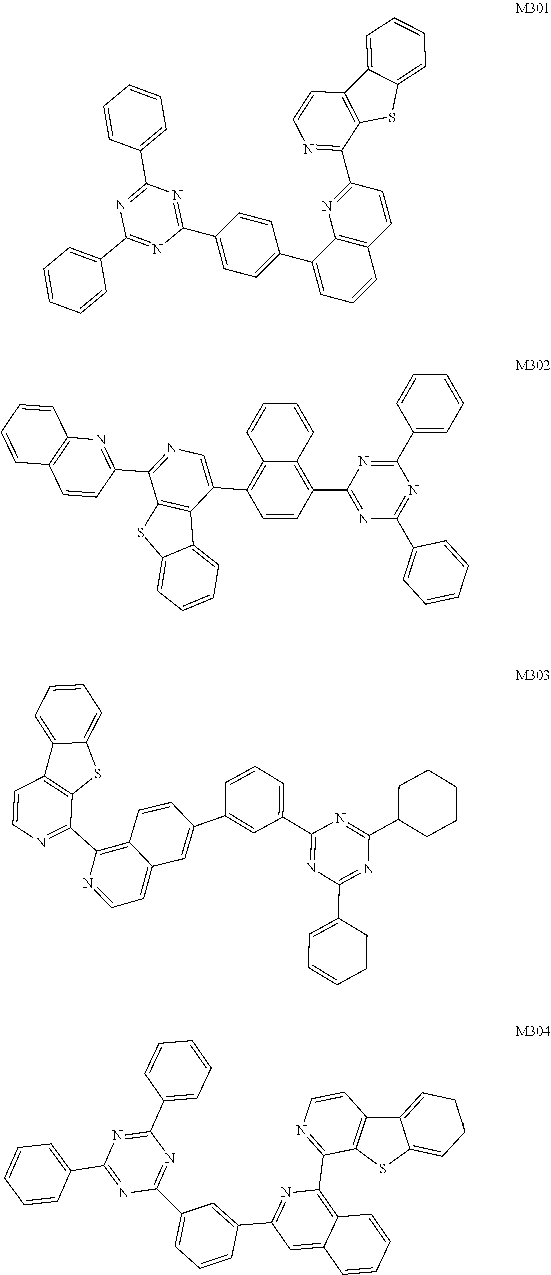 [SMILES: c1ccc(-c2nc(-c3ccccc3)nc(-c3ccc(-c4cccc5ccc(-c6nccc7c6sc6ccccc67)nc45)cc3)n2)cc1 ; c1ccc(-c2nc(-c3ccccc3)nc(-c3ccc(-c4cnc(-c5ccc6ccccc6n5)c5sc6ccccc6c45)c4ccccc34)n2)cc1 ; C1=CCCC(c2nc(-c3cccc(-c4ccc5c(-c6nccc7c6sc6ccccc67)nccc5c4)c3)nc(C3CCCCC3)n2)=C1 ; CC1C=c2sc3c(-c4nc(-c5cccc(-c6nc(-c7ccccc7)nc(-c7ccccc7)n6)c5)cc5ccccc45)nccc3c2=CC1]

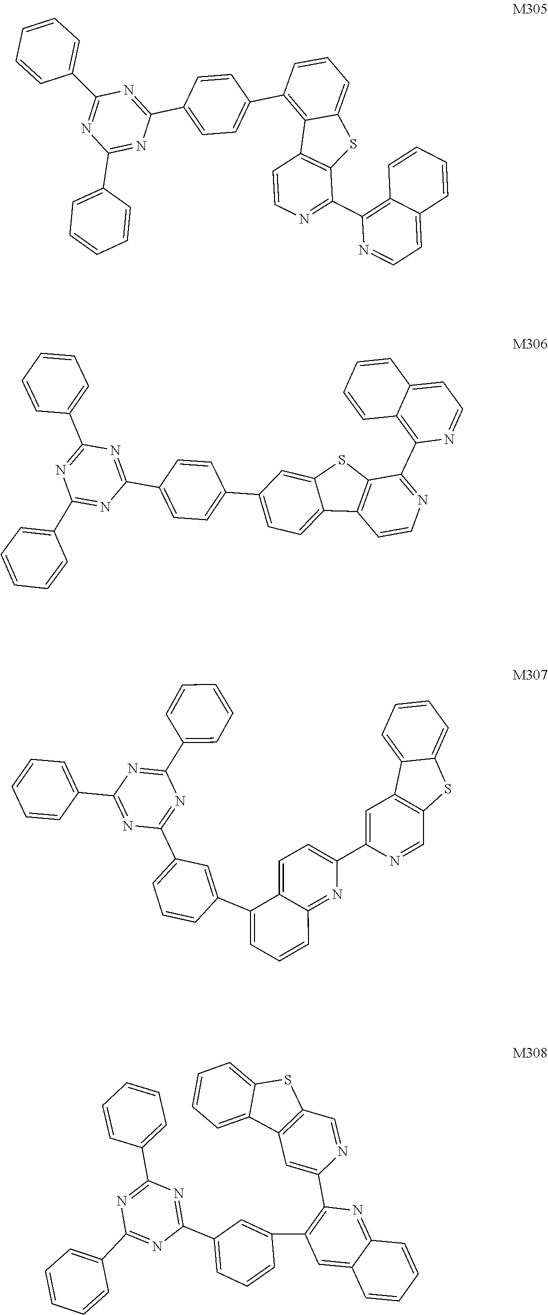 [SMILES: c1ccc(-c2nc(-c3ccccc3)nc(-c3ccc(-c4cccc5sc6c(-c7nccc8ccccc78)nccc6c45)cc3)n2)cc1 ; c1ccc(-c2nc(-c3ccccc3)nc(-c3ccc(-c4ccc5c(c4)sc4c(-c6nccc7ccccc67)nccc45)cc3)n2)cc1 ; c1ccc(-c2nc(-c3ccccc3)nc(-c3cccc(-c4cccc5nc(-c6cc7c(cn6)sc6ccccc67)ccc45)c3)n2)cc1 ; c1ccc(-c2nc(-c3ccccc3)nc(-c3cccc(-c4cc5ccccc5nc4-c4cc5c(cn4)sc4ccccc45)c3)n2)cc1]

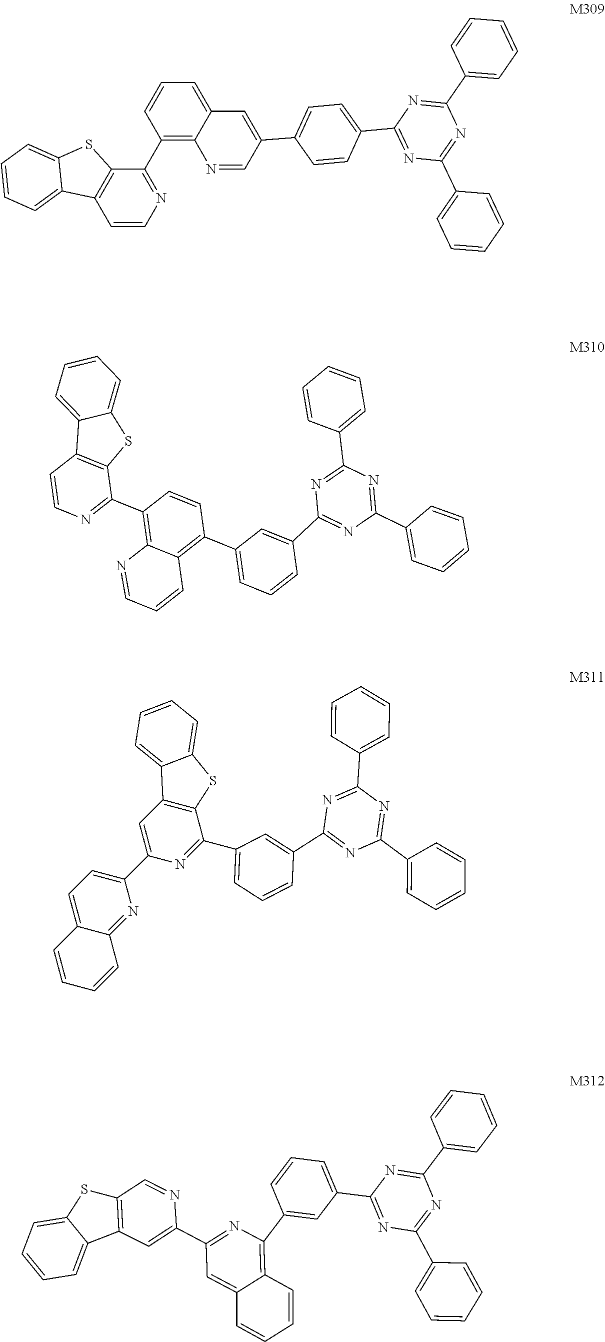 [SMILES: c1ccc(-c2nc(-c3ccccc3)nc(-c3ccc(-c4cnc5c(-c6nccc7c6sc6ccccc67)cccc5c4)cc3)n2)cc1 ; c1ccc(-c2nc(-c3ccccc3)nc(-c3cccc(-c4ccc(-c5nccc6c5sc5ccccc56)c5ncccc45)c3)n2)cc1 ; c1ccc(-c2nc(-c3ccccc3)nc(-c3cccc(-c4nc(-c5ccc6ccccc6n5)cc5c4sc4ccccc45)c3)n2)cc1 ; c1ccc(-c2nc(-c3ccccc3)nc(-c3cccc(-c4nc(-c5cc6c(cn5)sc5ccccc56)cc5ccccc45)c3)n2)cc1]

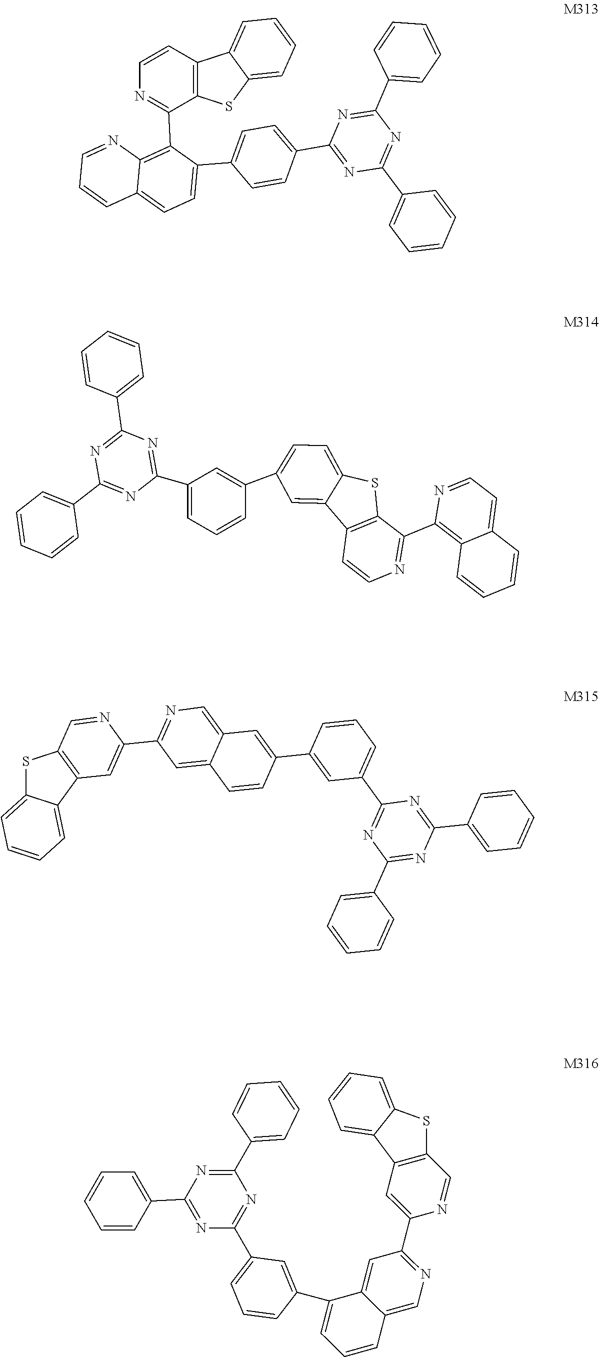 [SMILES: c1ccc(-c2nc(-c3ccccc3)nc(-c3ccc(-c4ccc5cccnc5c4-c4nccc5c4sc4ccccc45)cc3)n2)cc1 ; c1ccc(-c2nc(-c3ccccc3)nc(-c3cccc(-c4ccc5sc6c(-c7nccc8ccccc78)nccc6c5c4)c3)n2)cc1 ; c1ccc(-c2nc(-c3ccccc3)nc(-c3cccc(-c4ccc5cc(-c6cc7c(cn6)sc6ccccc67)ncc5c4)c3)n2)cc1 ; c1ccc(-c2nc(-c3ccccc3)nc(-c3cccc(-c4cccc5cnc(-c6cc7c(cn6)sc6ccccc67)cc45)c3)n2)cc1]

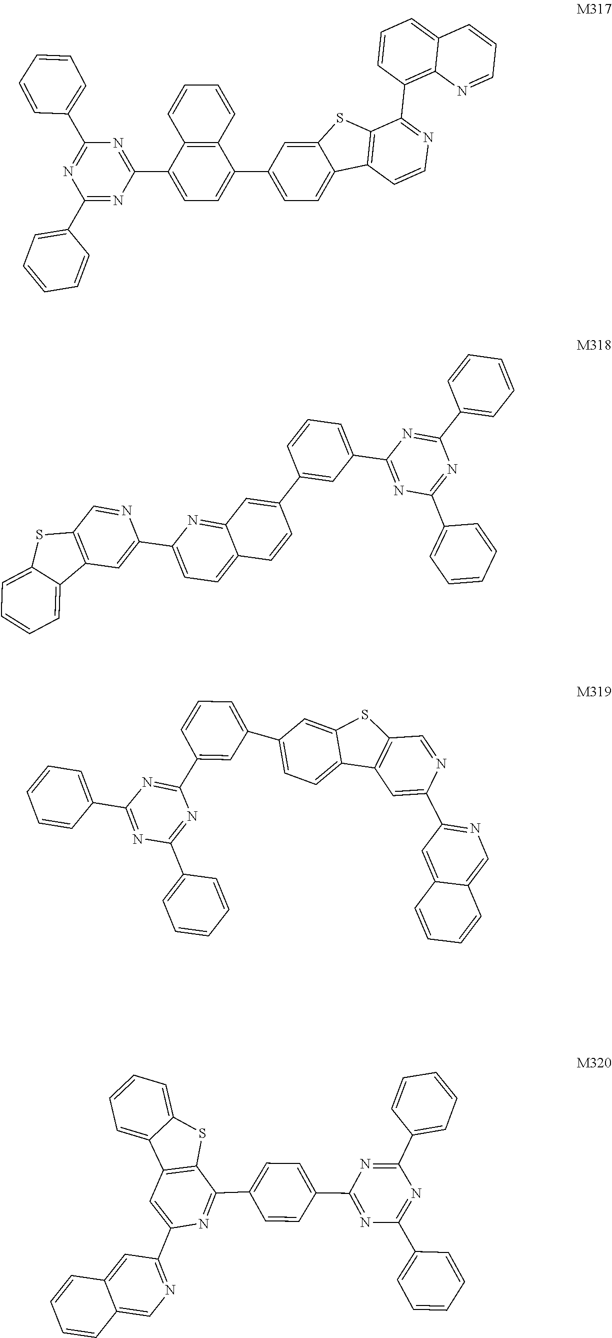 [SMILES: c1ccc(-c2nc(-c3ccccc3)nc(-c3ccc(-c4ccc5c(c4)sc4c(-c6cccc7cccnc67)nccc45)c4ccccc34)n2)cc1 ; c1ccc(-c2nc(-c3ccccc3)nc(-c3cccc(-c4ccc5ccc(-c6cc7c(cn6)sc6ccccc67)nc5c4)c3)n2)cc1 ; c1ccc(-c2nc(-c3ccccc3)nc(-c3cccc(-c4ccc5c(c4)sc4cnc(-c6cc7ccccc7cn6)cc45)c3)n2)cc1 ; c1ccc(-c2nc(-c3ccccc3)nc(-c3ccc(-c4nc(-c5cc6ccccc6cn5)cc5c4sc4ccccc45)cc3)n2)cc1]

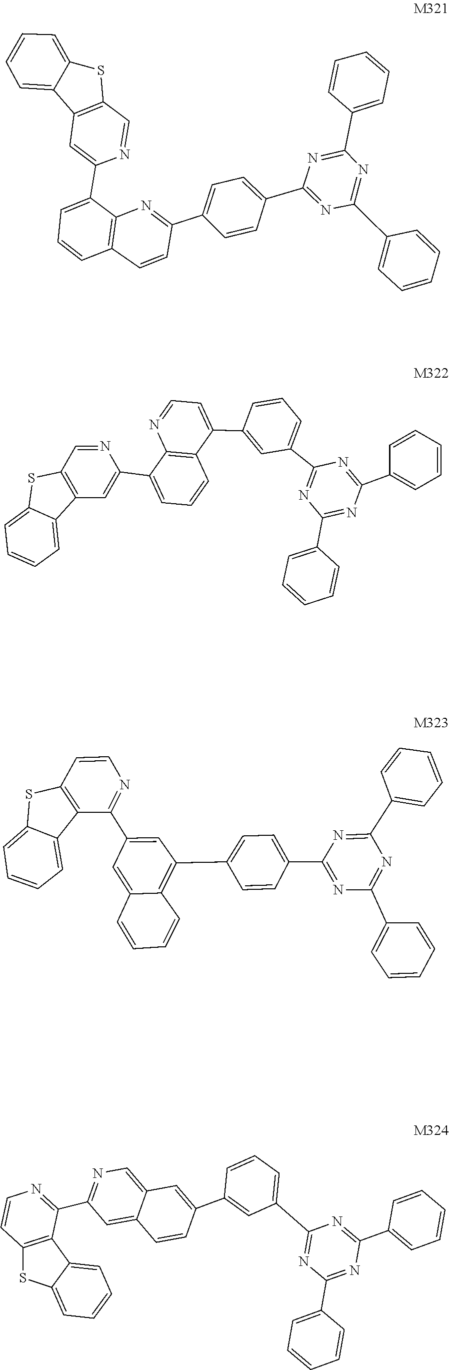 [SMILES: c1ccc(-c2nc(-c3ccccc3)nc(-c3ccc(-c4ccc5cccc(-c6cc7c(cn6)sc6ccccc67)c5n4)cc3)n2)cc1 ; c1ccc(-c2nc(-c3ccccc3)nc(-c3cccc(-c4ccnc5c(-c6cc7c(cn6)sc6ccccc67)cccc45)c3)n2)cc1 ; c1ccc(-c2nc(-c3ccccc3)nc(-c3ccc(-c4cc(-c5nccc6sc7ccccc7c56)cc5ccccc45)cc3)n2)cc1 ; c1ccc(-c2nc(-c3ccccc3)nc(-c3cccc(-c4ccc5cc(-c6nccc7sc8ccccc8c67)ncc5c4)c3)n2)cc1]

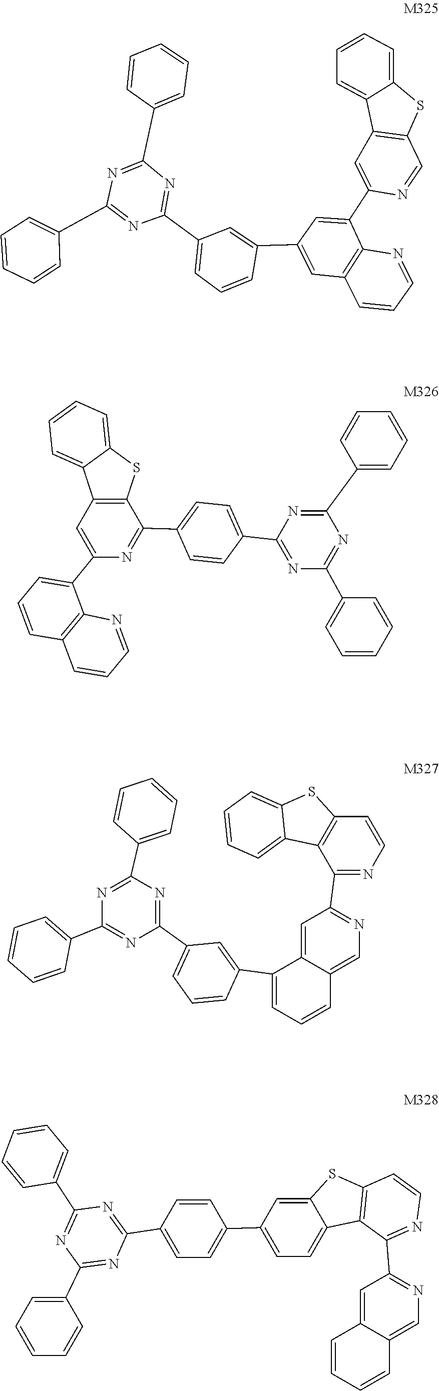 [SMILES: c1ccc(-c2nc(-c3ccccc3)nc(-c3cccc(-c4cc(-c5cc6c(cn5)sc5ccccc56)c5ncccc5c4)c3)n2)cc1 ; c1ccc(-c2nc(-c3ccccc3)nc(-c3ccc(-c4nc(-c5cccc6cccnc56)cc5c4sc4ccccc45)cc3)n2)cc1 ; c1ccc(-c2nc(-c3ccccc3)nc(-c3cccc(-c4cccc5cnc(-c6nccc7sc8ccccc8c67)cc45)c3)n2)cc1 ; c1ccc(-c2nc(-c3ccccc3)nc(-c3ccc(-c4ccc5c(c4)sc4ccnc(-c6cc7ccccc7cn6)c45)cc3)n2)cc1]

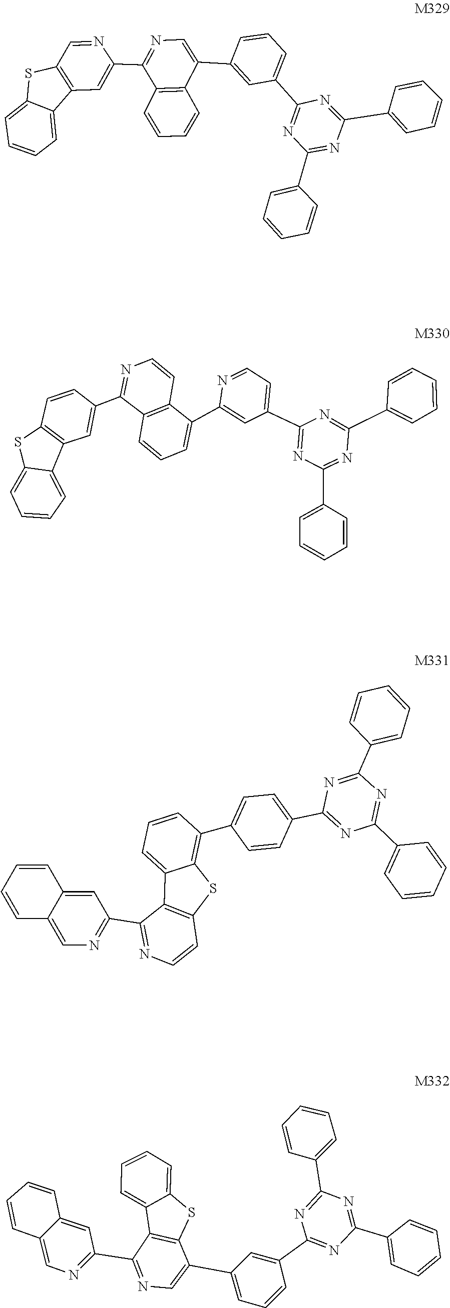 [SMILES: c1ccc(-c2nc(-c3ccccc3)nc(-c3cccc(-c4cnc(-c5cc6c(cn5)sc5ccccc56)c5ccccc45)c3)n2)cc1 ; c1ccc(-c2nc(-c3ccccc3)nc(-c3ccnc(-c4cccc5c(-c6ccc7sc8ccccc8c7c6)nccc45)c3)n2)cc1 ; c1ccc(-c2nc(-c3ccccc3)nc(-c3ccc(-c4cccc5c4sc4ccnc(-c6cc7ccccc7cn6)c45)cc3)n2)cc1 ; c1ccc(-c2nc(-c3ccccc3)nc(-c3cccc(-c4cnc(-c5cc6ccccc6cn5)c5c4sc4ccccc45)c3)n2)cc1]

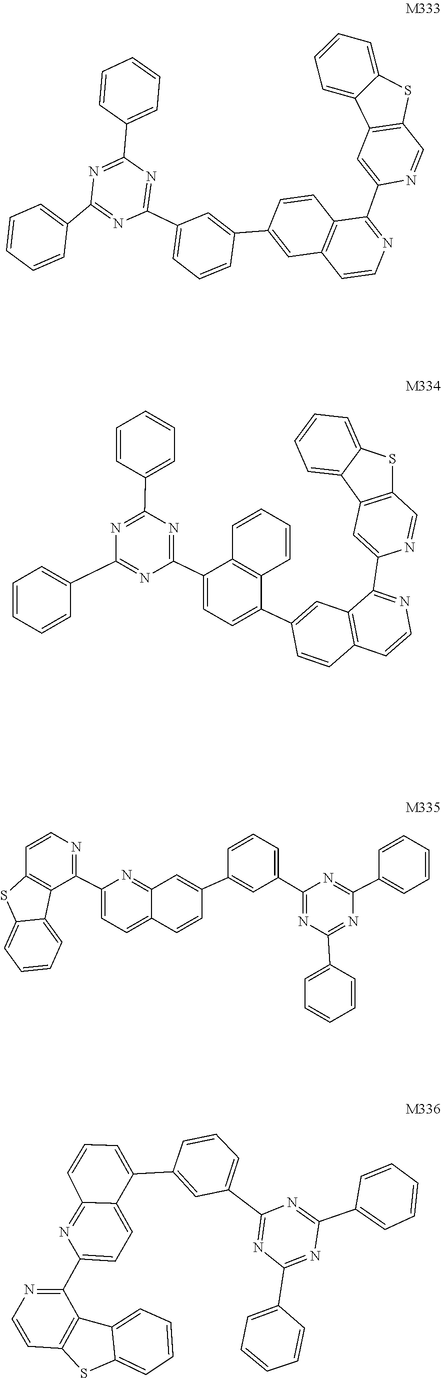 [SMILES: c1ccc(-c2nc(-c3ccccc3)nc(-c3cccc(-c4ccc5c(-c6cc7c(cn6)sc6ccccc67)nccc5c4)c3)n2)cc1 ; c1ccc(-c2nc(-c3ccccc3)nc(-c3ccc(-c4ccc5ccnc(-c6cc7c(cn6)sc6ccccc67)c5c4)c4ccccc34)n2)cc1 ; c1ccc(-c2nc(-c3ccccc3)nc(-c3cccc(-c4ccc5ccc(-c6nccc7sc8ccccc8c67)nc5c4)c3)n2)cc1 ; c1ccc(-c2nc(-c3ccccc3)nc(-c3cccc(-c4cccc5nc(-c6nccc7sc8ccccc8c67)ccc45)c3)n2)cc1]

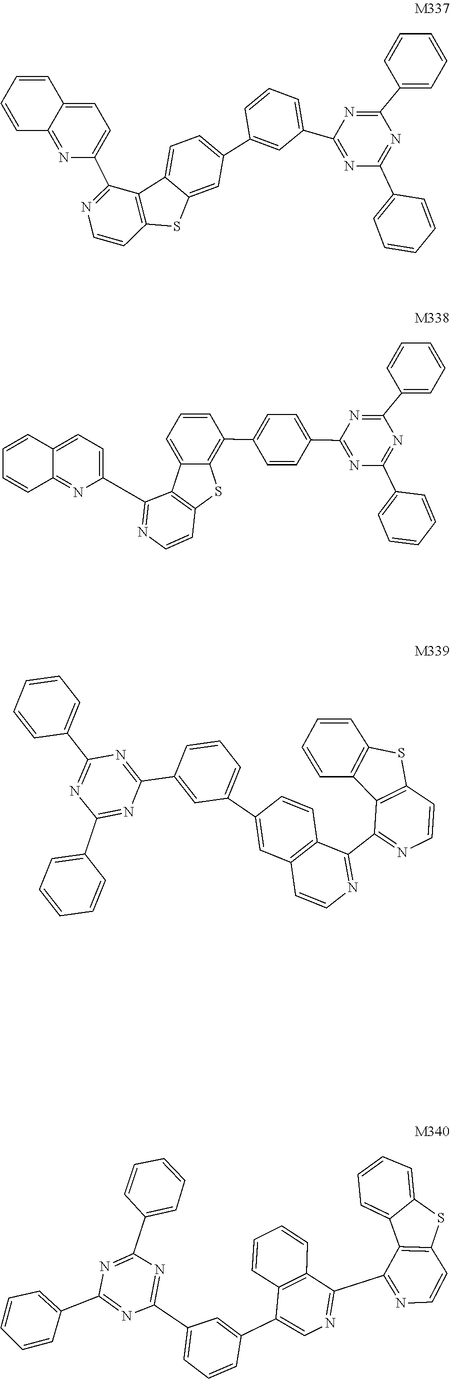 [SMILES: c1ccc(-c2nc(-c3ccccc3)nc(-c3cccc(-c4ccc5c(c4)sc4ccnc(-c6ccc7ccccc7n6)c45)c3)n2)cc1 ; c1ccc(-c2nc(-c3ccccc3)nc(-c3ccc(-c4cccc5c4sc4ccnc(-c6ccc7ccccc7n6)c45)cc3)n2)cc1 ; c1ccc(-c2nc(-c3ccccc3)nc(-c3cccc(-c4ccc5c(-c6nccc7sc8ccccc8c67)nccc5c4)c3)n2)cc1 ; c1ccc(-c2nc(-c3ccccc3)nc(-c3cccc(-c4cnc(-c5nccc6sc7ccccc7c56)c5ccccc45)c3)n2)cc1]

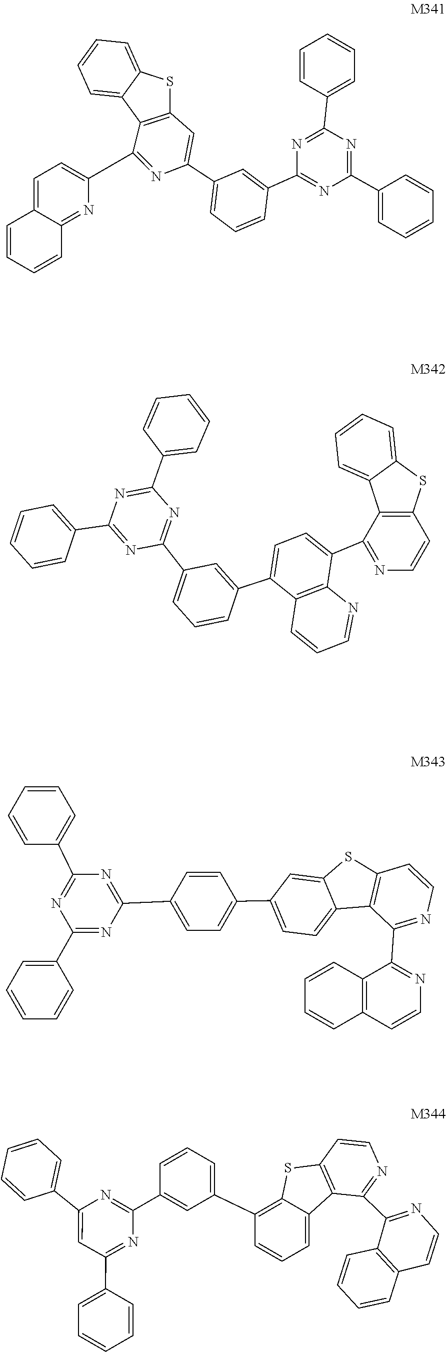 [SMILES: c1ccc(-c2nc(-c3ccccc3)nc(-c3cccc(-c4cc5sc6ccccc6c5c(-c5ccc6ccccc6n5)n4)c3)n2)cc1 ; c1ccc(-c2nc(-c3ccccc3)nc(-c3cccc(-c4ccc(-c5nccc6sc7ccccc7c56)c5ncccc45)c3)n2)cc1 ; c1ccc(-c2nc(-c3ccccc3)nc(-c3ccc(-c4ccc5c(c4)sc4ccnc(-c6nccc7ccccc67)c45)cc3)n2)cc1 ; c1ccc(-c2cc(-c3ccccc3)nc(-c3cccc(-c4cccc5c4sc4ccnc(-c6nccc7ccccc67)c45)c3)n2)cc1]

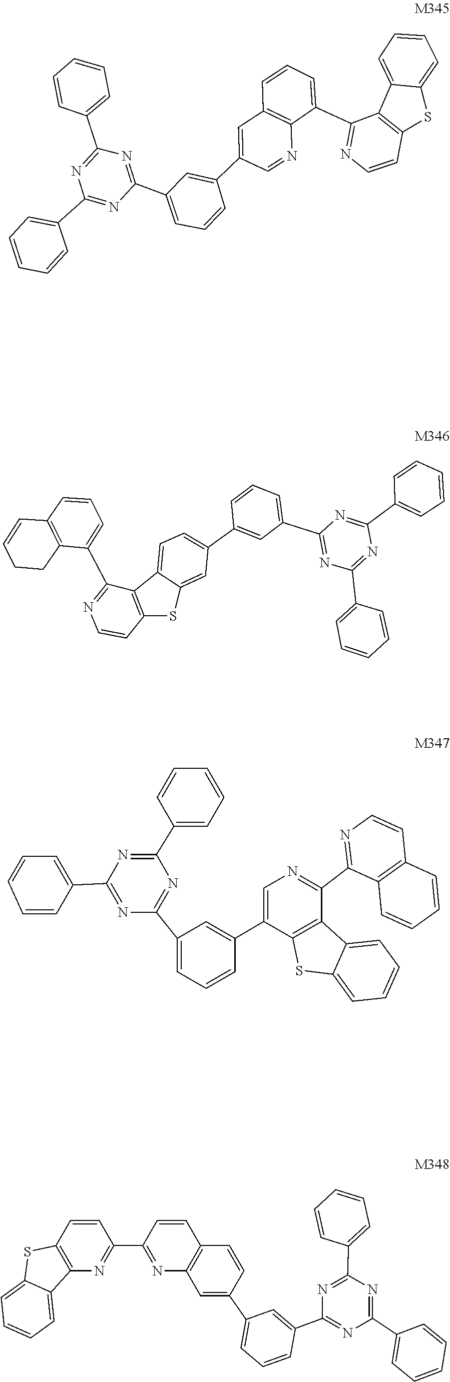

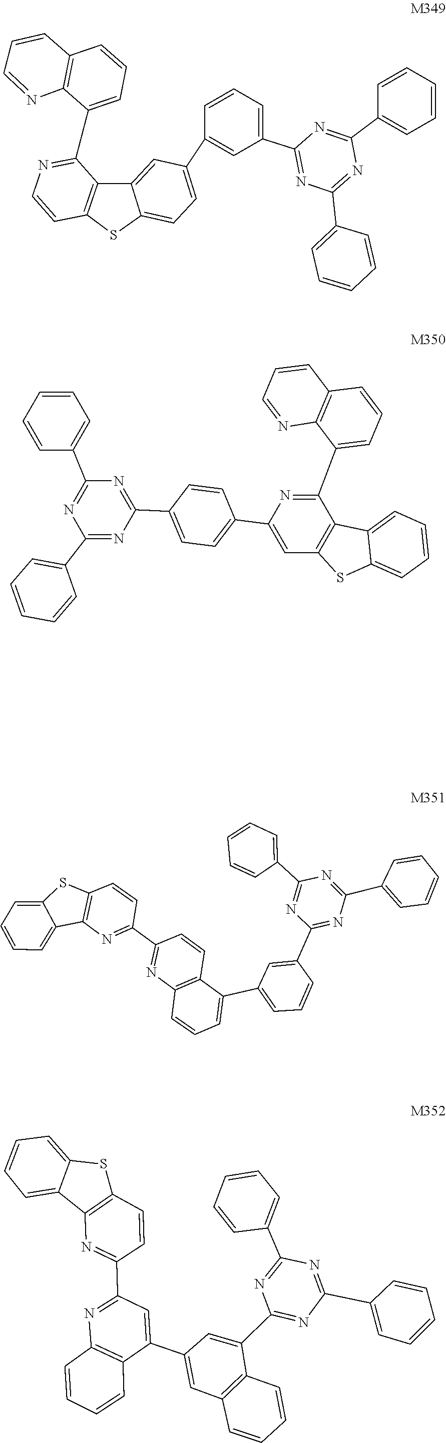 [SMILES: c1ccc(-c2nc(-c3ccccc3)nc(-c3cccc(-c4ccc5sc6ccnc(-c7cccc8cccnc78)c6c5c4)c3)n2)cc1 ; c1ccc(-c2nc(-c3ccccc3)nc(-c3ccc(-c4cc5sc6ccccc6c5c(-c5cccc6cccnc56)n4)cc3)n2)cc1 ; c1ccc(-c2nc(-c3ccccc3)nc(-c3cccc(-c4cccc5nc(-c6ccc7sc8ccccc8c7n6)ccc45)c3)n2)cc1 ; c1ccc(-c2nc(-c3ccccc3)nc(-c3cc(-c4cc(-c5ccc6sc7ccccc7c6n5)nc5ccccc45)cc4ccccc34)n2)cc1]

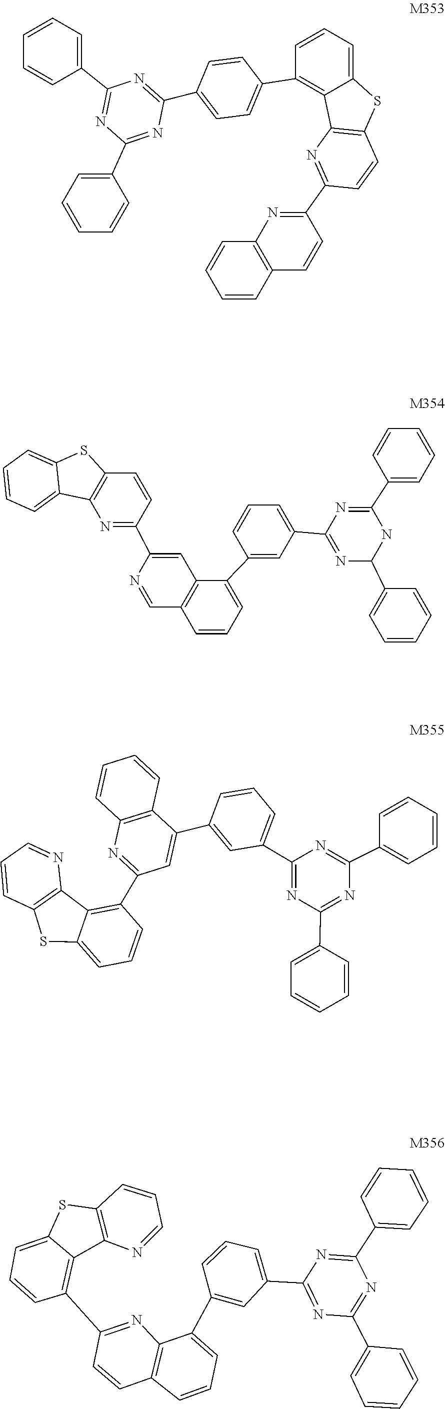

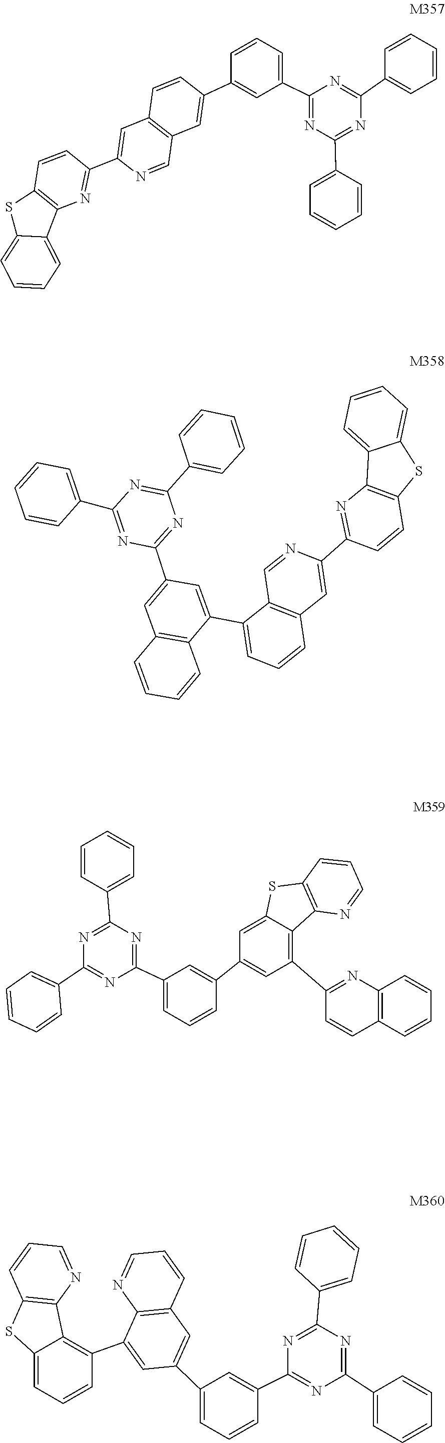 [SMILES: c1ccc(-c2nc(-c3ccccc3)nc(-c3cccc(-c4ccc5cc(-c6ccc7sc8ccccc8c7n6)ncc5c4)c3)n2)cc1 ; c1ccc(-c2nc(-c3ccccc3)nc(-c3cc(-c4cccc5cc(-c6ccc7sc8ccccc8c7n6)ncc45)c4ccccc4c3)n2)cc1 ; c1ccc(-c2nc(-c3ccccc3)nc(-c3cccc(-c4cc(-c5ccc6ccccc6n5)c5c(c4)sc4cccnc45)c3)n2)cc1 ; c1ccc(-c2nc(-c3ccccc3)nc(-c3cccc(-c4cc(-c5cccc6sc7cccnc7c56)c5ncccc5c4)c3)n2)cc1]

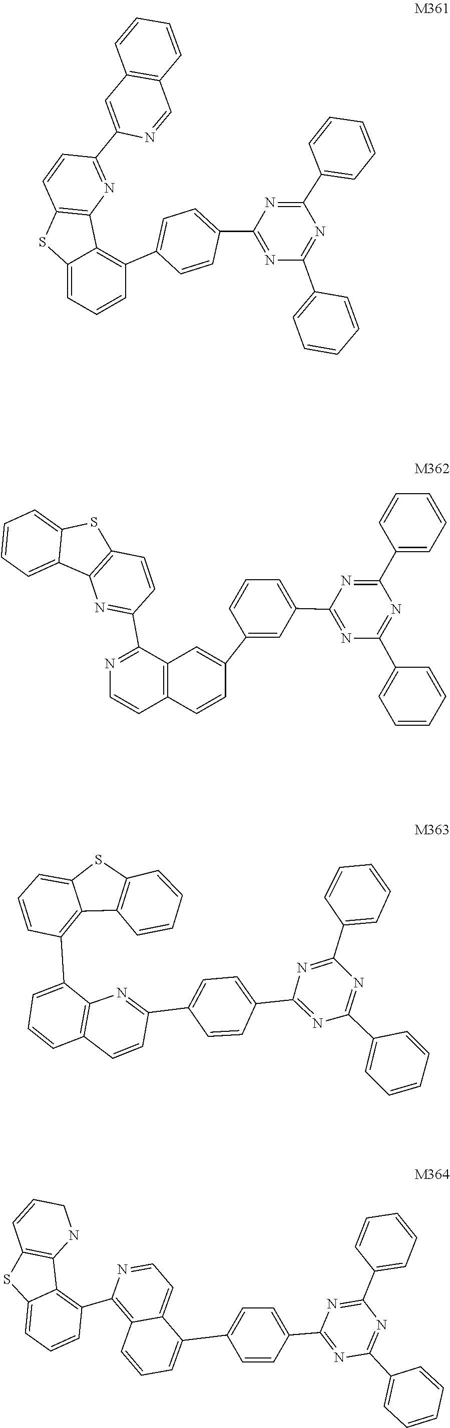

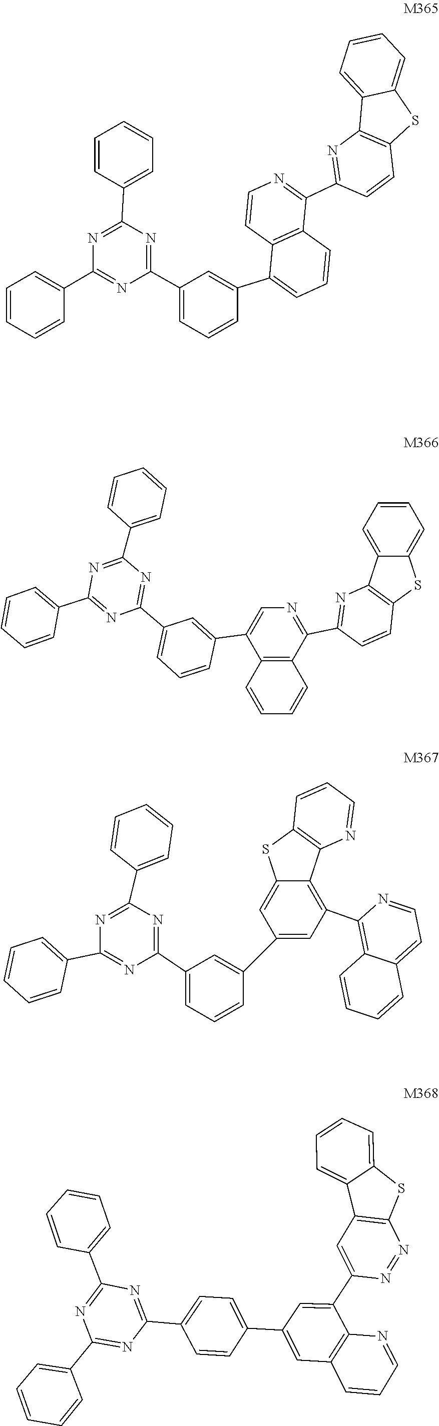 [SMILES: c1ccc(-c2nc(-c3ccccc3)nc(-c3cccc(-c4cccc5c(-c6ccc7sc8ccccc8c7n6)nccc45)c3)n2)cc1 ; c1ccc(-c2nc(-c3ccccc3)nc(-c3cccc(-c4cnc(-c5ccc6sc7ccccc7c6n5)c5ccccc45)c3)n2)cc1 ; c1ccc(-c2nc(-c3ccccc3)nc(-c3cccc(-c4cc(-c5nccc6ccccc56)c5c(c4)sc4cccnc45)c3)n2)cc1 ; c1ccc(-c2nc(-c3ccccc3)nc(-c3ccc(-c4cc(-c5cc6c(nn5)sc5ccccc56)c5ncccc5c4)cc3)n2)cc1]

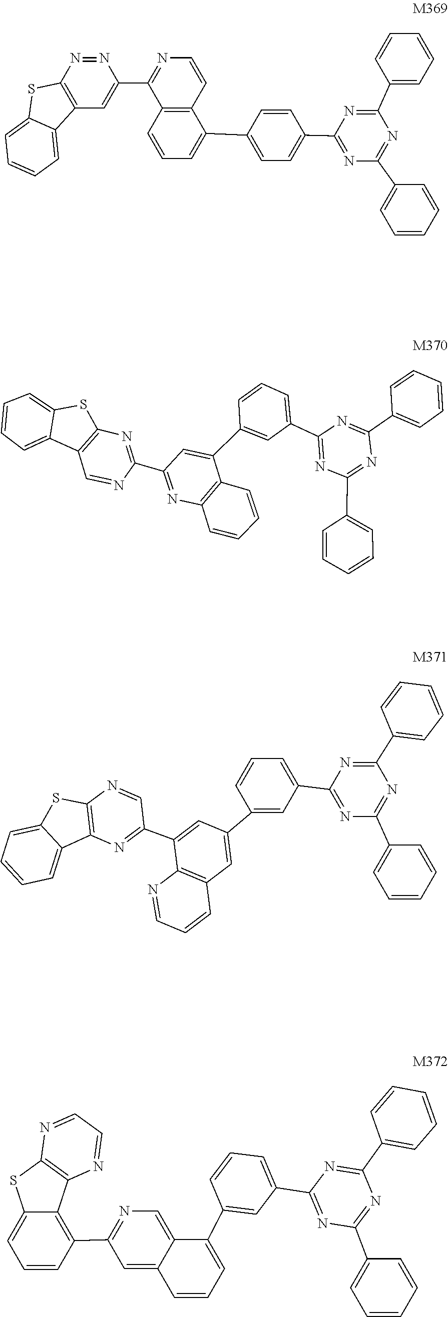 [SMILES: c1ccc(-c2nc(-c3ccccc3)nc(-c3ccc(-c4cccc5c(-c6cc7c(nn6)sc6ccccc67)nccc45)cc3)n2)cc1 ; c1ccc(-c2nc(-c3ccccc3)nc(-c3cccc(-c4cc(-c5ncc6c(n5)sc5ccccc56)nc5ccccc45)c3)n2)cc1 ; c1ccc(-c2nc(-c3ccccc3)nc(-c3cccc(-c4cc(-c5cnc6sc7ccccc7c6n5)c5ncccc5c4)c3)n2)cc1 ; c1ccc(-c2nc(-c3ccccc3)nc(-c3cccc(-c4cccc5cc(-c6cccc7sc8nccnc8c67)ncc45)c3)n2)cc1]

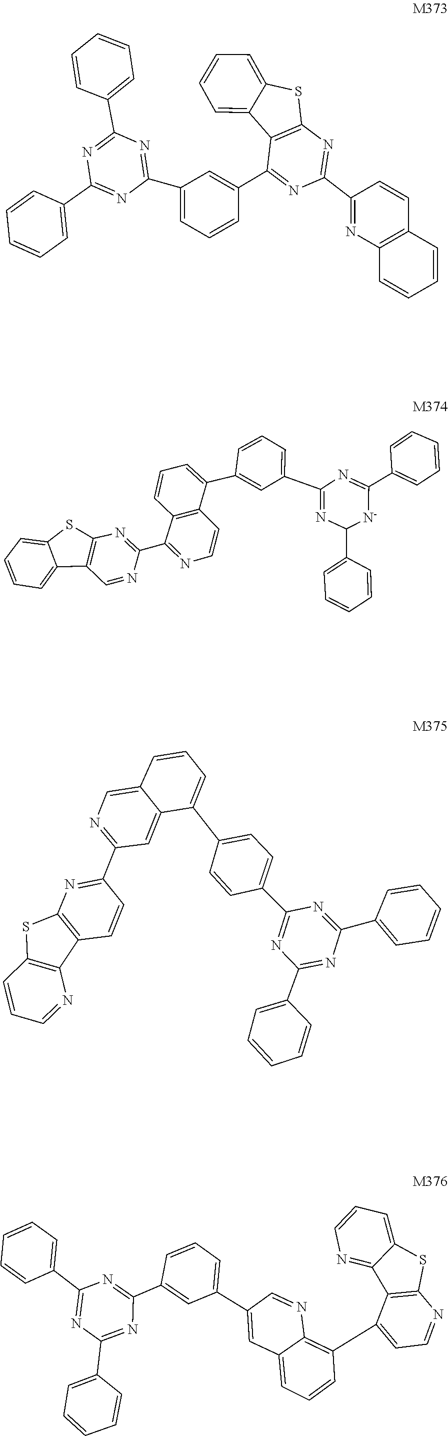 [SMILES: c1ccc(-c2nc(-c3ccccc3)nc(-c3cccc(-c4nc(-c5ccc6ccccc6n5)nc5sc6ccccc6c45)c3)n2)cc1 ; c1ccc(C2=NC(c3cccc(-c4cccc5c(-c6ncc7c(n6)sc6ccccc67)nccc45)c3)=NC(c3ccccc3)[N-]2)cc1 ; c1ccc(-c2nc(-c3ccccc3)nc(-c3ccc(-c4cccc5cnc(-c6ccc7c(n6)sc6cccnc67)cc45)cc3)n2)cc1 ; c1ccc(-c2nc(-c3ccccc3)nc(-c3cccc(-c4cnc5c(-c6ccnc7sc8cccnc8c67)cccc5c4)c3)n2)cc1]

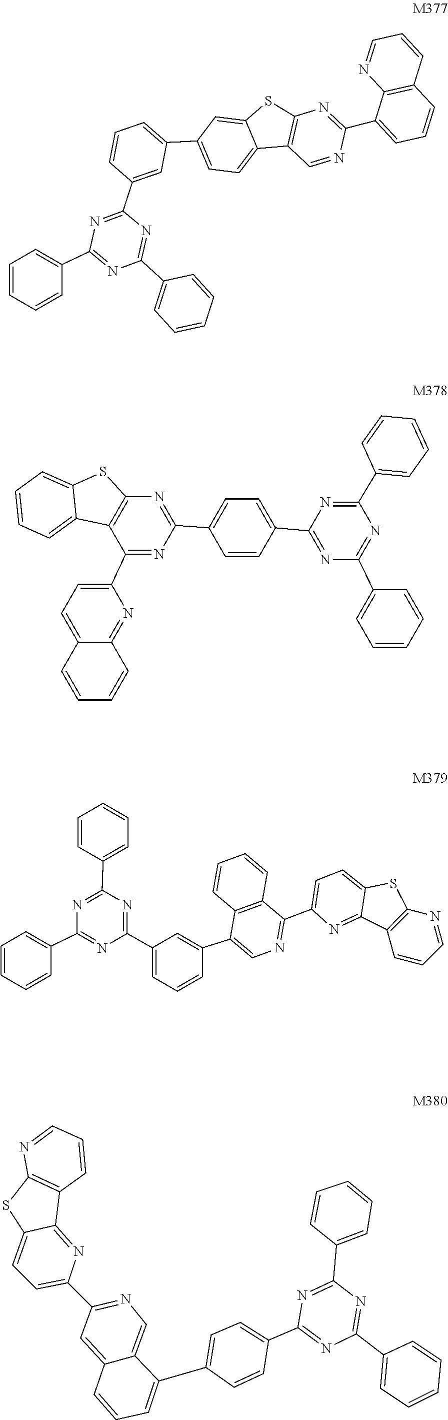 [SMILES: c1ccc(-c2nc(-c3ccccc3)nc(-c3cccc(-c4ccc5c(c4)sc4nc(-c6cccc7cccnc67)ncc45)c3)n2)cc1 ; c1ccc(-c2nc(-c3ccccc3)nc(-c3ccc(-c4nc(-c5ccc6ccccc6n5)c5c(n4)sc4ccccc45)cc3)n2)cc1 ; c1ccc(-c2nc(-c3ccccc3)nc(-c3cccc(-c4cnc(-c5ccc6sc7ncccc7c6n5)c5ccccc45)c3)n2)cc1 ; c1ccc(-c2nc(-c3ccccc3)nc(-c3ccc(-c4cccc5cc(-c6ccc7sc8ncccc8c7n6)ncc45)cc3)n2)cc1]

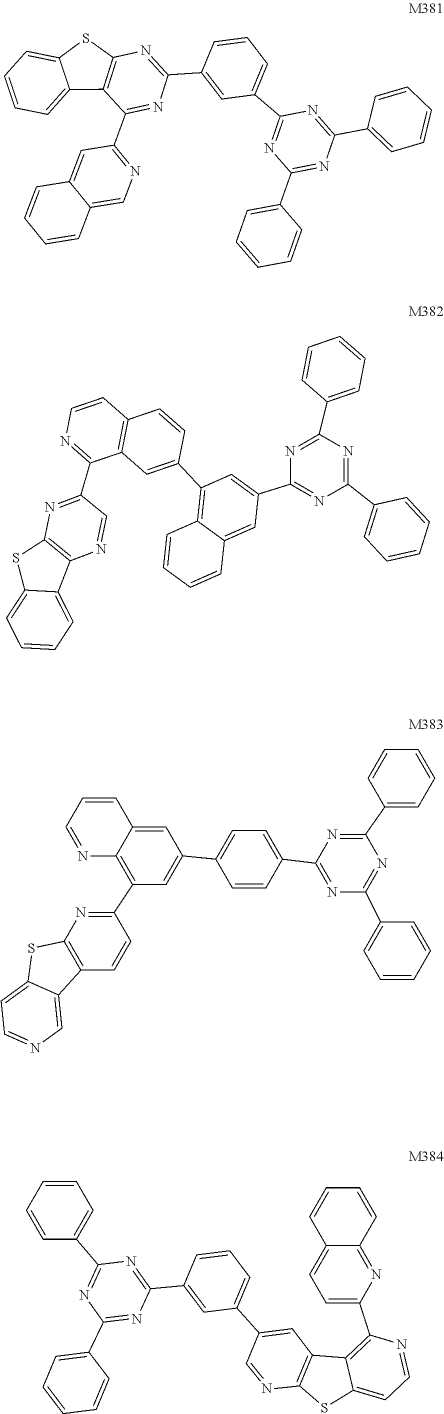 [SMILES: c1ccc(-c2nc(-c3ccccc3)nc(-c3cccc(-c4nc(-c5cc6ccccc6cn5)c5c(n4)sc4ccccc45)c3)n2)cc1 ; c1ccc(-c2nc(-c3ccccc3)nc(-c3cc(-c4ccc5ccnc(-c6cnc7c(n6)sc6ccccc67)c5c4)c4ccccc4c3)n2)cc1 ; c1ccc(-c2nc(-c3ccccc3)nc(-c3ccc(-c4cc(-c5ccc6c(n5)sc5ccncc56)c5ncccc5c4)cc3)n2)cc1 ; c1ccc(-c2nc(-c3ccccc3)nc(-c3cccc(-c4cnc5sc6ccnc(-c7ccc8ccccc8n7)c6c5c4)c3)n2)cc1]

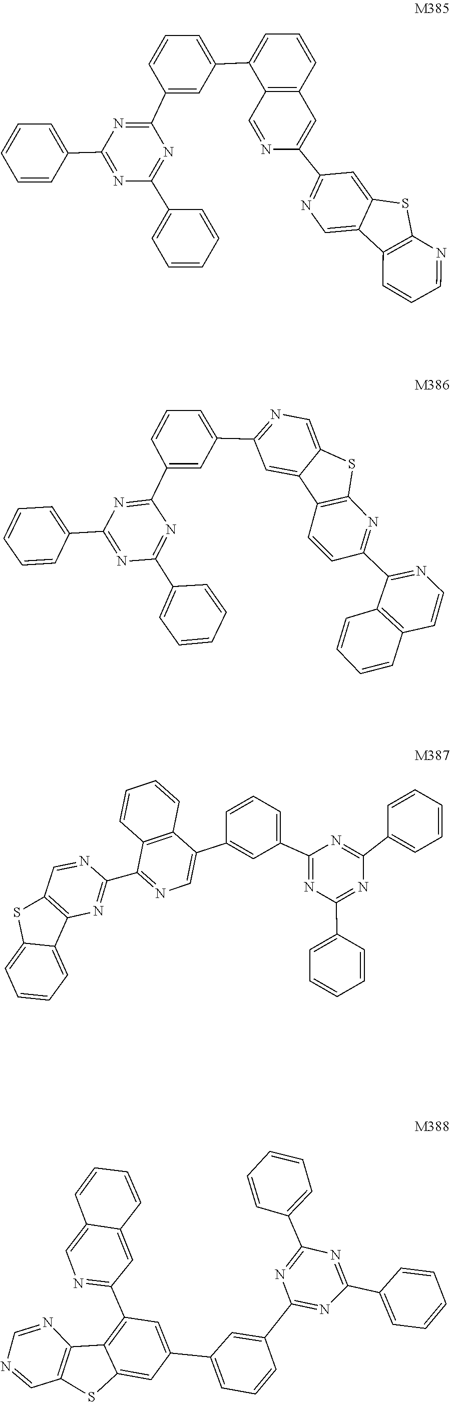

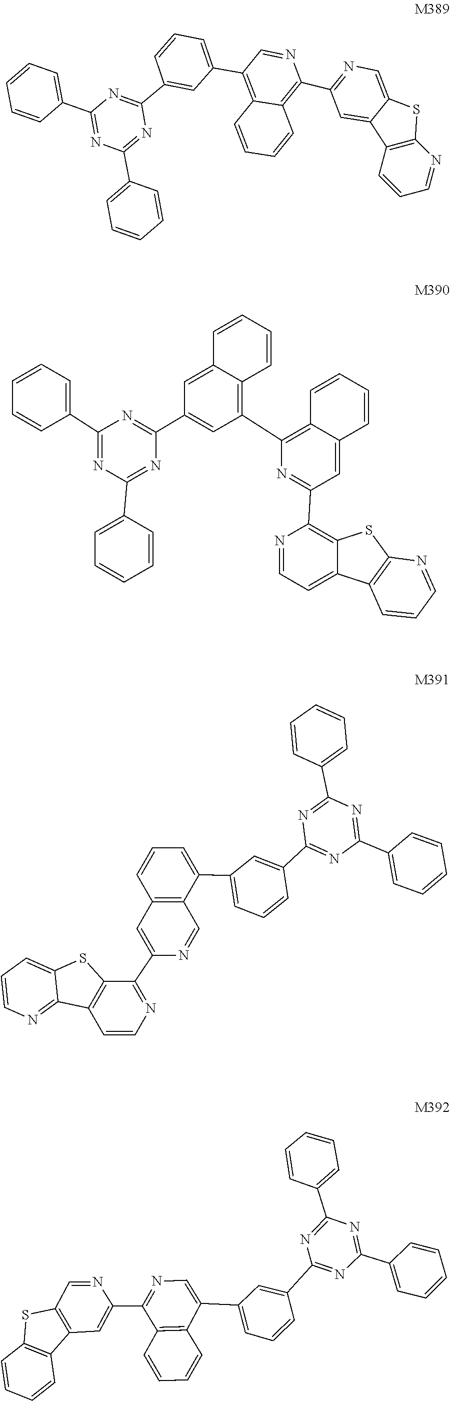 [SMILES: c1ccc(-c2nc(-c3ccccc3)nc(-c3cccc(-c4cnc(-c5cc6c(cn5)sc5ncccc56)c5ccccc45)c3)n2)cc1 ; c1ccc(-c2nc(-c3ccccc3)nc(-c3cc(-c4nc(-c5nccc6c5sc5ncccc56)cc5ccccc45)c4ccccc4c3)n2)cc1 ; c1ccc(-c2nc(-c3ccccc3)nc(-c3cccc(-c4cccc5cc(-c6nccc7c6sc6cccnc67)ncc45)c3)n2)cc1 ; c1ccc(-c2nc(-c3ccccc3)nc(-c3cccc(-c4cnc(-c5cc6c(cn5)sc5ccccc56)c5ccccc45)c3)n2)cc1]

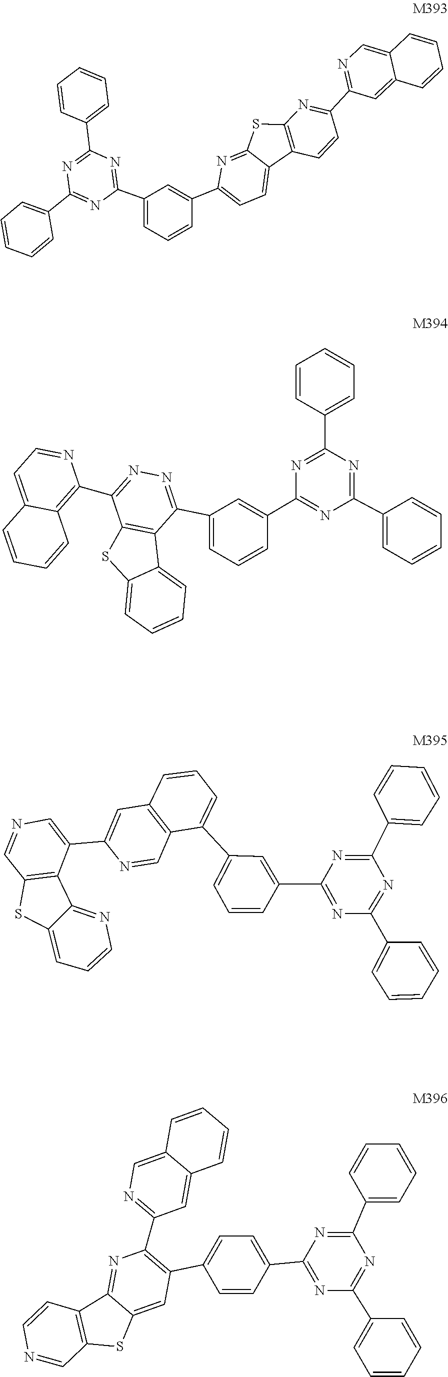 [SMILES: c1ccc(-c2nc(-c3ccccc3)nc(-c3cccc(-c4ccc5c(n4)sc4nc(-c6cc7ccccc7cn6)ccc45)c3)n2)cc1 ; c1ccc(-c2nc(-c3ccccc3)nc(-c3cccc(-c4nnc(-c5nccc6ccccc56)c5sc6ccccc6c45)c3)n2)cc1 ; c1ccc(-c2nc(-c3ccccc3)nc(-c3cccc(-c4cccc5cc(-c6cncc7sc8cccnc8c67)ncc45)c3)n2)cc1 ; c1ccc(-c2nc(-c3ccccc3)nc(-c3ccc(-c4cc5sc6cnccc6c5nc4-c4cc5ccccc5cn4)cc3)n2)cc1]

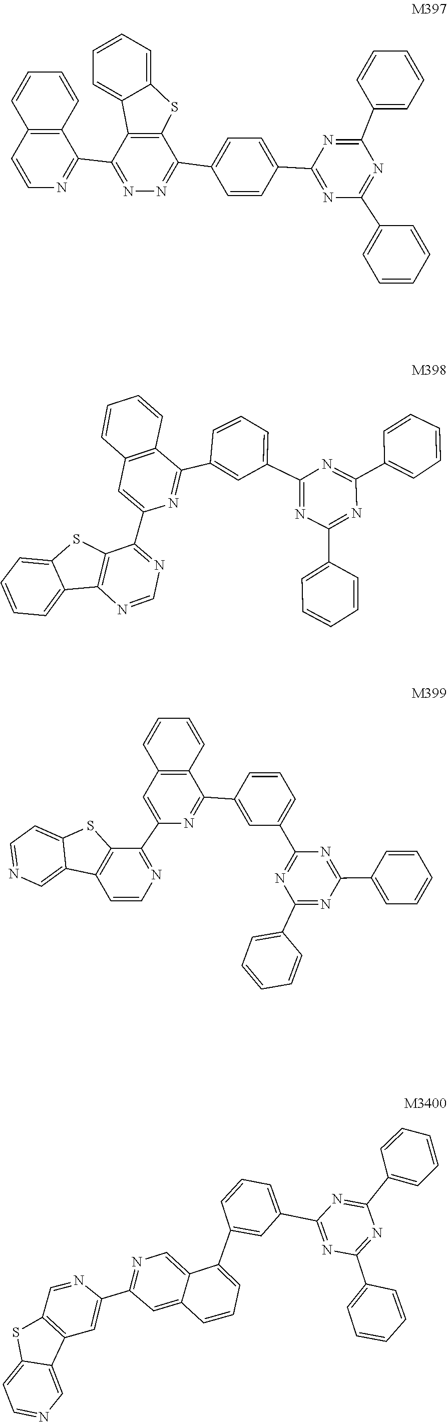 [SMILES: c1ccc(-c2nc(-c3ccccc3)nc(-c3ccc(-c4nnc(-c5nccc6ccccc56)c5c4sc4ccccc45)cc3)n2)cc1 ; c1ccc(-c2nc(-c3ccccc3)nc(-c3cccc(-c4nc(-c5ncnc6c5sc5ccccc56)cc5ccccc45)c3)n2)cc1 ; c1ccc(-c2nc(-c3ccccc3)nc(-c3cccc(-c4nc(-c5nccc6c5sc5ccncc56)cc5ccccc45)c3)n2)cc1 ; c1ccc(-c2nc(-c3ccccc3)nc(-c3cccc(-c4cccc5cc(-c6cc7c(cn6)sc6ccncc67)ncc45)c3)n2)cc1]

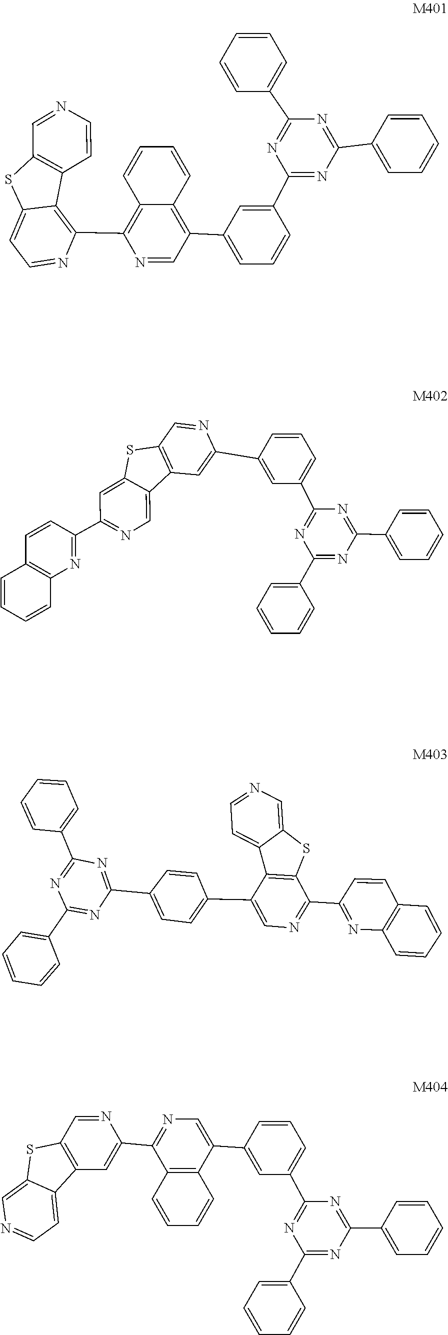 [SMILES: c1ccc(-c2nc(-c3ccccc3)nc(-c3cccc(-c4cnc(-c5nccc6sc7cnccc7c56)c5ccccc45)c3)n2)cc1 ; c1ccc(-c2nc(-c3ccccc3)nc(-c3cccc(-c4cc5c(cn4)sc4cc(-c6ccc7ccccc7n6)ncc45)c3)n2)cc1 ; c1ccc(-c2nc(-c3ccccc3)nc(-c3ccc(-c4cnc(-c5ccc6ccccc6n5)c5sc6cnccc6c45)cc3)n2)cc1 ; c1ccc(-c2nc(-c3ccccc3)nc(-c3cccc(-c4cnc(-c5cc6c(cn5)sc5cnccc56)c5ccccc45)c3)n2)cc1]

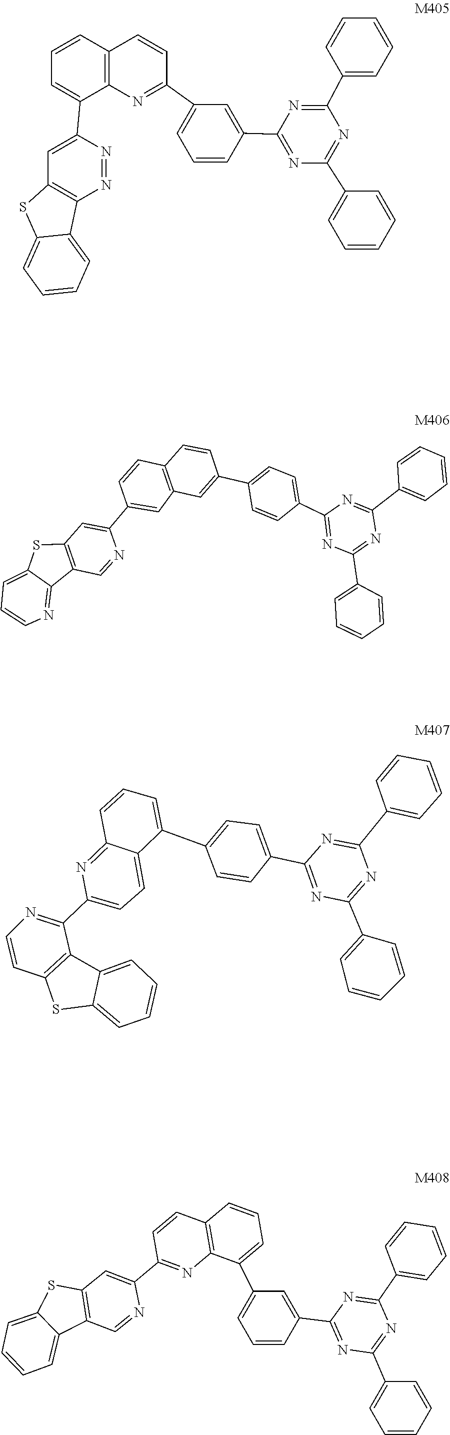 [SMILES: c1ccc(-c2nc(-c3ccccc3)nc(-c3cccc(-c4ccc5cccc(-c6cc7sc8ccccc8c7nn6)c5n4)c3)n2)cc1 ; c1ccc(-c2nc(-c3ccccc3)nc(-c3ccc(-c4ccc5ccc(-c6cc7sc8cccnc8c7cn6)cc5c4)cc3)n2)cc1 ; c1ccc(-c2nc(-c3ccccc3)nc(-c3ccc(-c4cccc5nc(-c6nccc7sc8ccccc8c67)ccc45)cc3)n2)cc1 ; c1ccc(-c2nc(-c3ccccc3)nc(-c3cccc(-c4cccc5ccc(-c6cc7sc8ccccc8c7cn6)nc45)c3)n2)cc1]

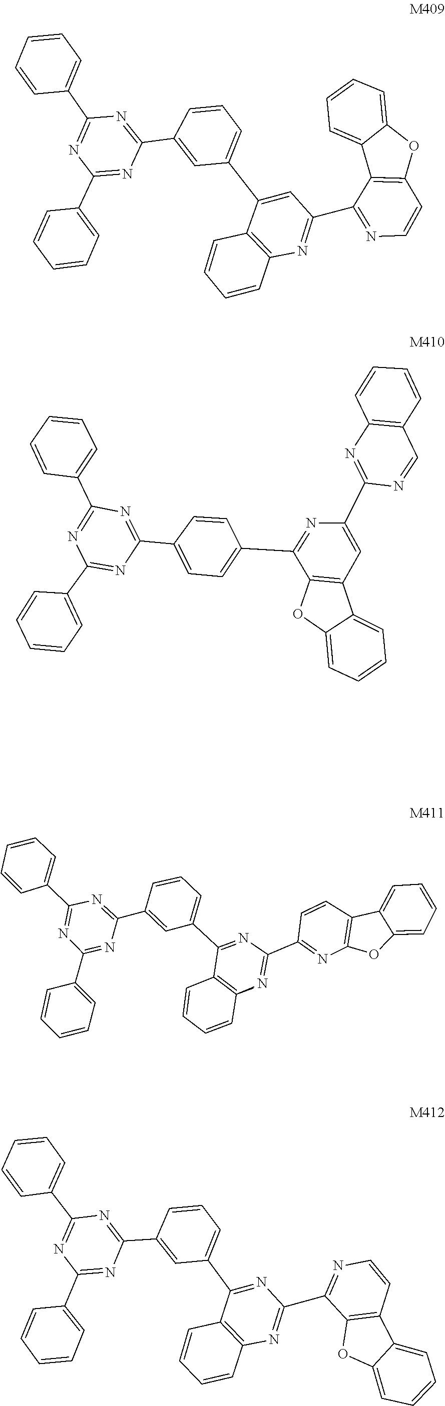

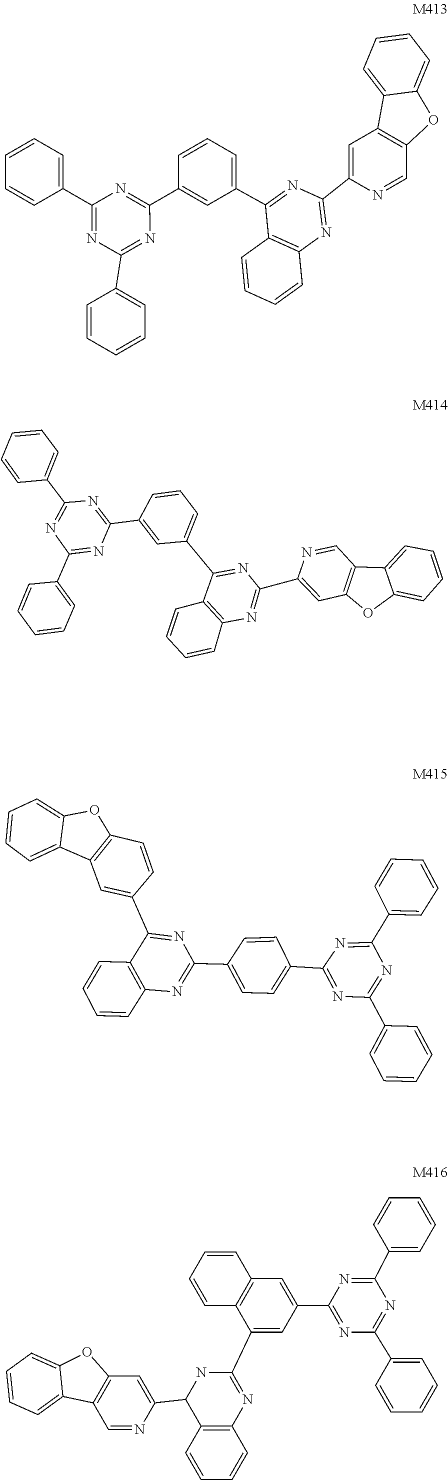 [SMILES: c1ccc(-c2nc(-c3ccccc3)nc(-c3cccc(-c4nc(-c5cc6c(cn5)oc5ccccc56)nc5ccccc45)c3)n2)cc1 ; c1ccc(-c2nc(-c3ccccc3)nc(-c3cccc(-c4nc(-c5cc6oc7ccccc7c6cn5)nc5ccccc45)c3)n2)cc1 ; c1ccc(-c2nc(-c3ccccc3)nc(-c3ccc(-c4nc(-c5ccc6oc7ccccc7c6c5)c5ccccc5n4)cc3)n2)cc1 ; c1ccc(-c2nc(-c3ccccc3)nc(-c3cc(C4=Nc5ccccc5C(c5cc6oc7ccccc7c6cn5)N4)c4ccccc4c3)n2)cc1]

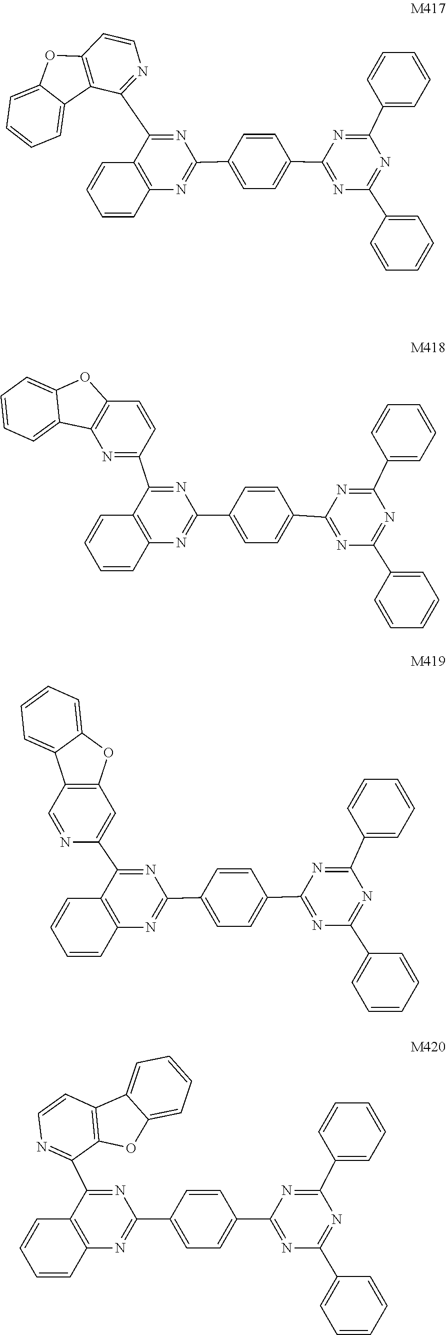 [SMILES: c1ccc(-c2nc(-c3ccccc3)nc(-c3ccc(-c4nc(-c5nccc6oc7ccccc7c56)c5ccccc5n4)cc3)n2)cc1 ; c1ccc(-c2nc(-c3ccccc3)nc(-c3ccc(-c4nc(-c5ccc6oc7ccccc7c6n5)c5ccccc5n4)cc3)n2)cc1 ; c1ccc(-c2nc(-c3ccccc3)nc(-c3ccc(-c4nc(-c5cc6oc7ccccc7c6cn5)c5ccccc5n4)cc3)n2)cc1 ; c1ccc(-c2nc(-c3ccccc3)nc(-c3ccc(-c4nc(-c5nccc6c5oc5ccccc56)c5ccccc5n4)cc3)n2)cc1]

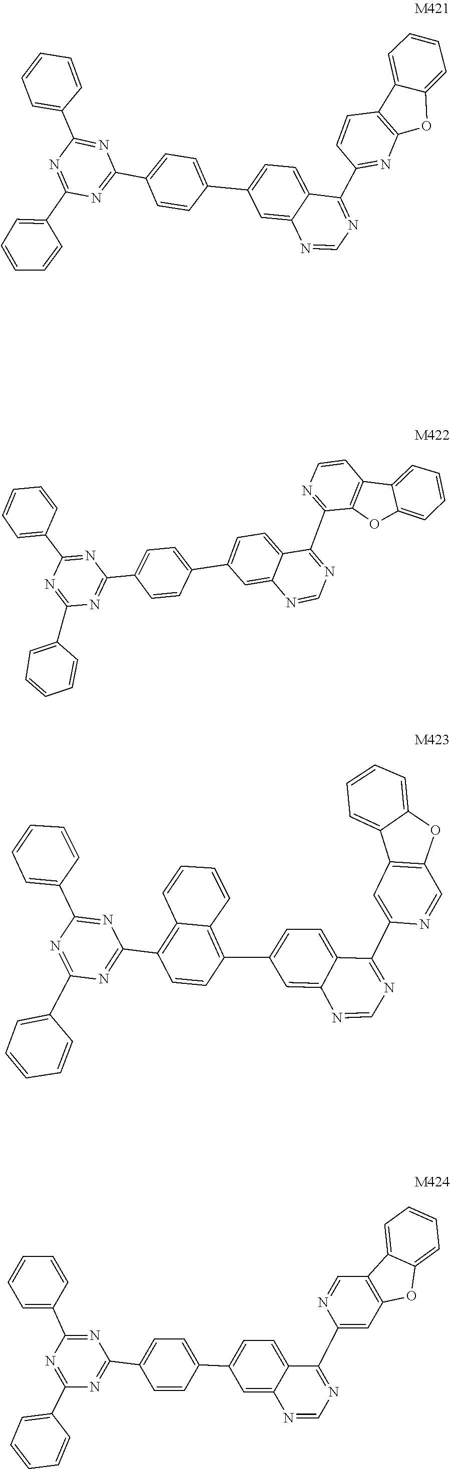 [SMILES: c1ccc(-c2nc(-c3ccccc3)nc(-c3ccc(-c4ccc5c(-c6ccc7c(n6)oc6ccccc67)ncnc5c4)cc3)n2)cc1 ; c1ccc(-c2nc(-c3ccccc3)nc(-c3ccc(-c4ccc5c(-c6nccc7c6oc6ccccc67)ncnc5c4)cc3)n2)cc1 ; c1ccc(-c2nc(-c3ccccc3)nc(-c3ccc(-c4ccc5c(-c6cc7c(cn6)oc6ccccc67)ncnc5c4)c4ccccc34)n2)cc1 ; c1ccc(-c2nc(-c3ccccc3)nc(-c3ccc(-c4ccc5c(-c6cc7oc8ccccc8c7cn6)ncnc5c4)cc3)n2)cc1]

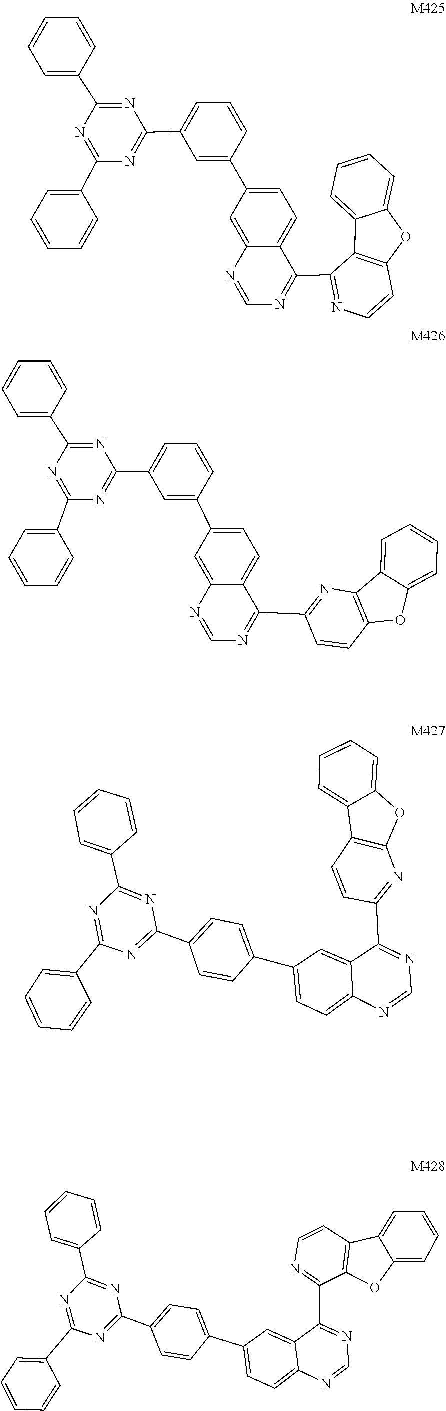 [SMILES: c1ccc(-c2nc(-c3ccccc3)nc(-c3cccc(-c4ccc5c(-c6nccc7oc8ccccc8c67)ncnc5c4)c3)n2)cc1 ; c1ccc(-c2nc(-c3ccccc3)nc(-c3cccc(-c4ccc5c(-c6ccc7oc8ccccc8c7n6)ncnc5c4)c3)n2)cc1 ; c1ccc(-c2nc(-c3ccccc3)nc(-c3ccc(-c4ccc5ncnc(-c6ccc7c(n6)oc6ccccc67)c5c4)cc3)n2)cc1 ; c1ccc(-c2nc(-c3ccccc3)nc(-c3ccc(-c4ccc5ncnc(-c6nccc7c6oc6ccccc67)c5c4)cc3)n2)cc1]

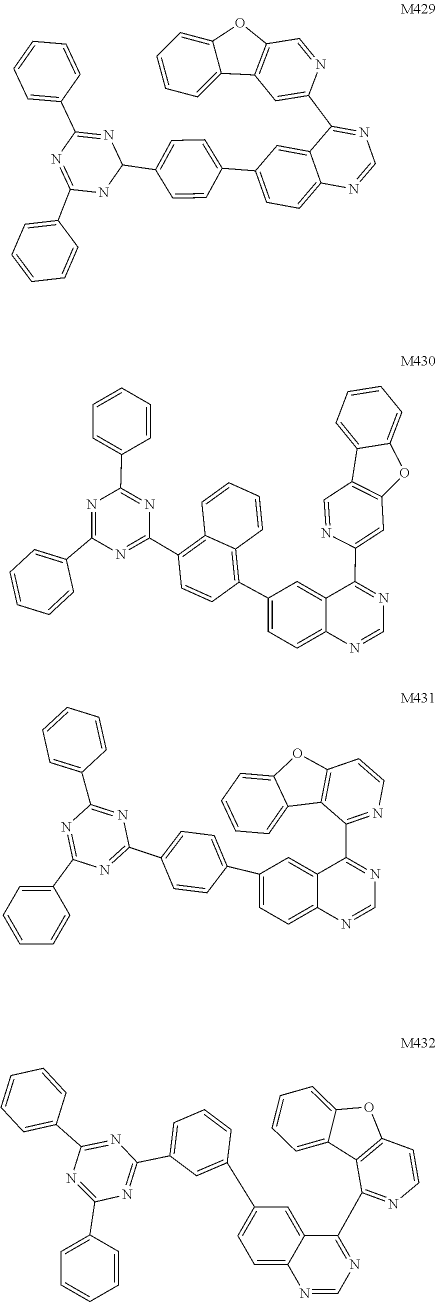 [SMILES: c1ccc(C2=NC(c3ccc(-c4ccc5ncnc(-c6cc7c(cn6)oc6ccccc67)c5c4)cc3)NC(c3ccccc3)=N2)cc1 ; c1ccc(-c2nc(-c3ccccc3)nc(-c3ccc(-c4ccc5ncnc(-c6cc7oc8ccccc8c7cn6)c5c4)c4ccccc34)n2)cc1 ; c1ccc(-c2nc(-c3ccccc3)nc(-c3ccc(-c4ccc5ncnc(-c6nccc7oc8ccccc8c67)c5c4)cc3)n2)cc1 ; c1ccc(-c2nc(-c3ccccc3)nc(-c3cccc(-c4ccc5ncnc(-c6nccc7oc8ccccc8c67)c5c4)c3)n2)cc1]

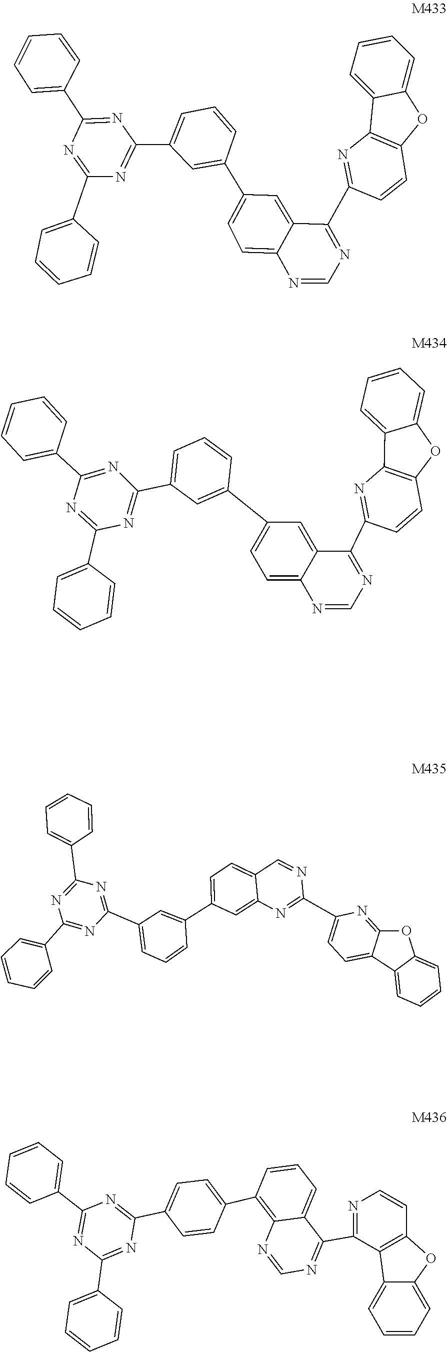 [SMILES: c1ccc(-c2nc(-c3ccccc3)nc(-c3cccc(-c4ccc5ncnc(-c6ccc7oc8ccccc8c7n6)c5c4)c3)n2)cc1 ; c1ccc(-c2nc(-c3ccccc3)nc(-c3cccc(-c4ccc5ncnc(-c6ccc7oc8ccccc8c7n6)c5c4)c3)n2)cc1 ; c1ccc(-c2nc(-c3ccccc3)nc(-c3cccc(-c4ccc5cnc(-c6ccc7c(n6)oc6ccccc67)nc5c4)c3)n2)cc1 ; c1ccc(-c2nc(-c3ccccc3)nc(-c3ccc(-c4cccc5c(-c6nccc7oc8ccccc8c67)ncnc45)cc3)n2)cc1]

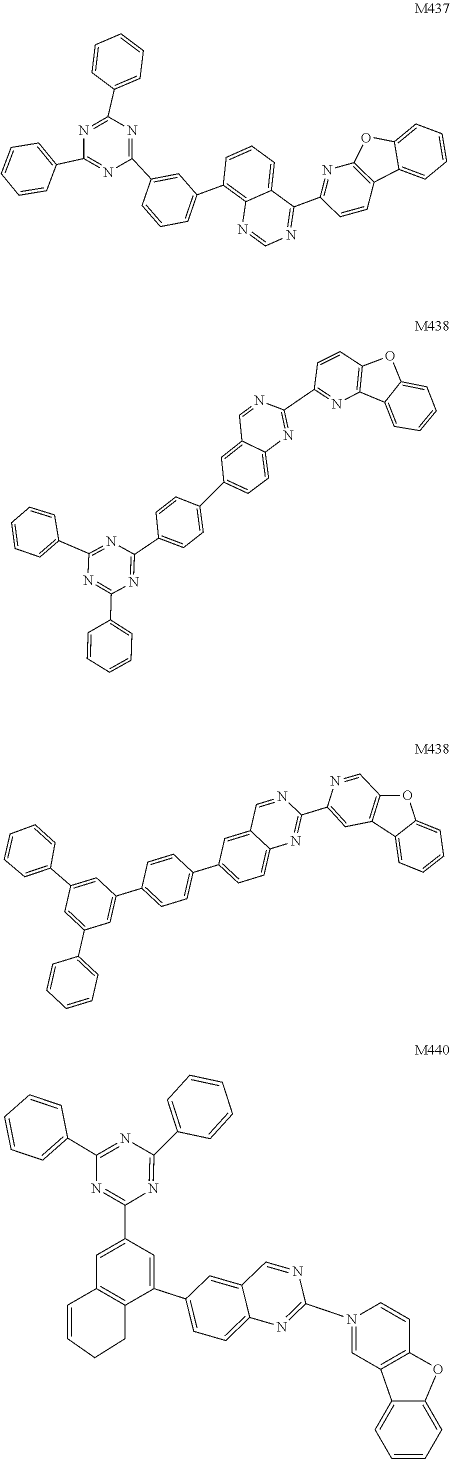

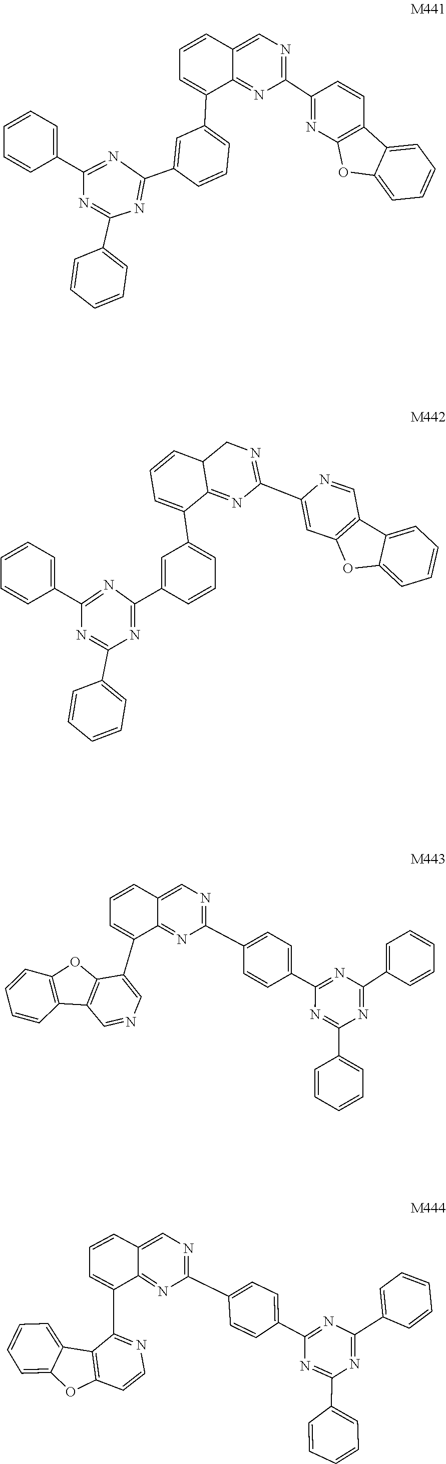

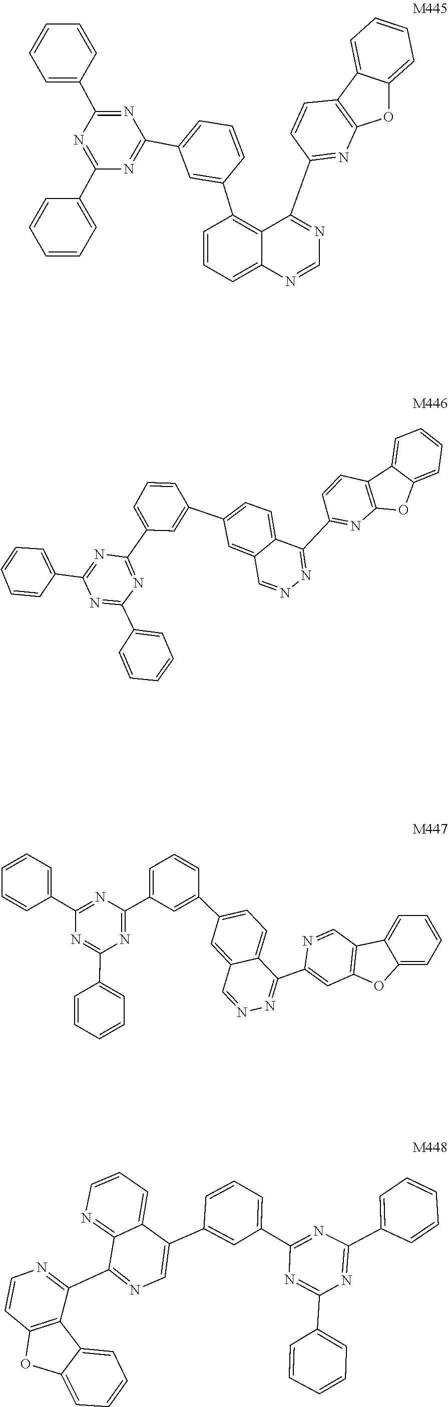 [SMILES: c1ccc(-c2nc(-c3ccccc3)nc(-c3cccc(-c4cccc5ncnc(-c6ccc7c(n6)oc6ccccc67)c45)c3)n2)cc1 ; c1ccc(-c2nc(-c3ccccc3)nc(-c3cccc(-c4ccc5c(-c6ccc7c(n6)oc6ccccc67)nncc5c4)c3)n2)cc1 ; c1ccc(-c2nc(-c3ccccc3)nc(-c3cccc(-c4ccc5c(-c6cc7oc8ccccc8c7cn6)nncc5c4)c3)n2)cc1 ; c1ccc(-c2nc(-c3ccccc3)nc(-c3cccc(-c4cnc(-c5nccc6oc7ccccc7c56)c5ncccc45)c3)n2)cc1]

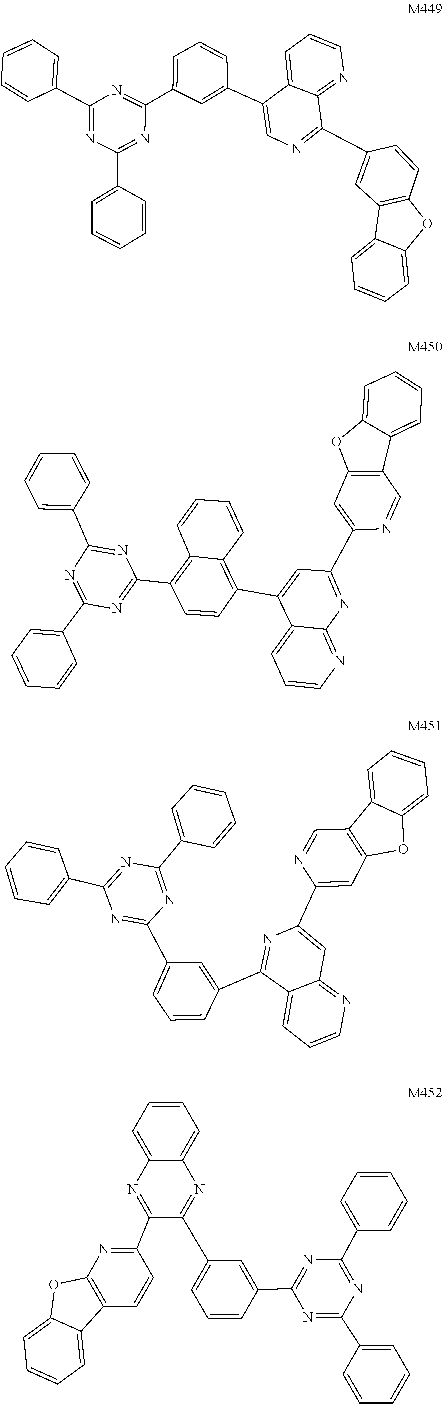 [SMILES: c1ccc(-c2nc(-c3ccccc3)nc(-c3cccc(-c4cnc(-c5ccc6oc7ccccc7c6c5)c5ncccc45)c3)n2)cc1 ; c1ccc(-c2nc(-c3ccccc3)nc(-c3ccc(-c4cc(-c5cc6oc7ccccc7c6cn5)nc5ncccc45)c4ccccc34)n2)cc1 ; c1ccc(-c2nc(-c3ccccc3)nc(-c3cccc(-c4nc(-c5cc6oc7ccccc7c6cn5)cc5ncccc45)c3)n2)cc1 ; c1ccc(-c2nc(-c3ccccc3)nc(-c3cccc(-c4nc5ccccc5nc4-c4ccc5c(n4)oc4ccccc45)c3)n2)cc1]

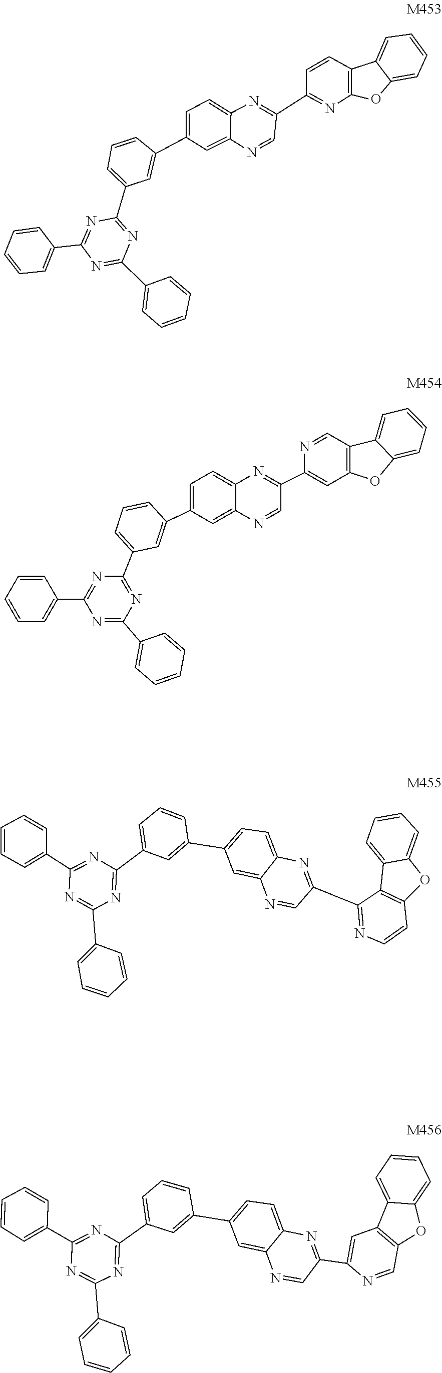 [SMILES: c1ccc(-c2nc(-c3ccccc3)nc(-c3cccc(-c4ccc5nc(-c6ccc7c(n6)oc6ccccc67)cnc5c4)c3)n2)cc1 ; c1ccc(-c2nc(-c3ccccc3)nc(-c3cccc(-c4ccc5nc(-c6cc7oc8ccccc8c7cn6)cnc5c4)c3)n2)cc1 ; c1ccc(-c2nc(-c3ccccc3)nc(-c3cccc(-c4ccc5nc(-c6nccc7oc8ccccc8c67)cnc5c4)c3)n2)cc1 ; c1ccc(-c2nc(-c3ccccc3)nc(-c3cccc(-c4ccc5nc(-c6cc7c(cn6)oc6ccccc67)cnc5c4)c3)n2)cc1]

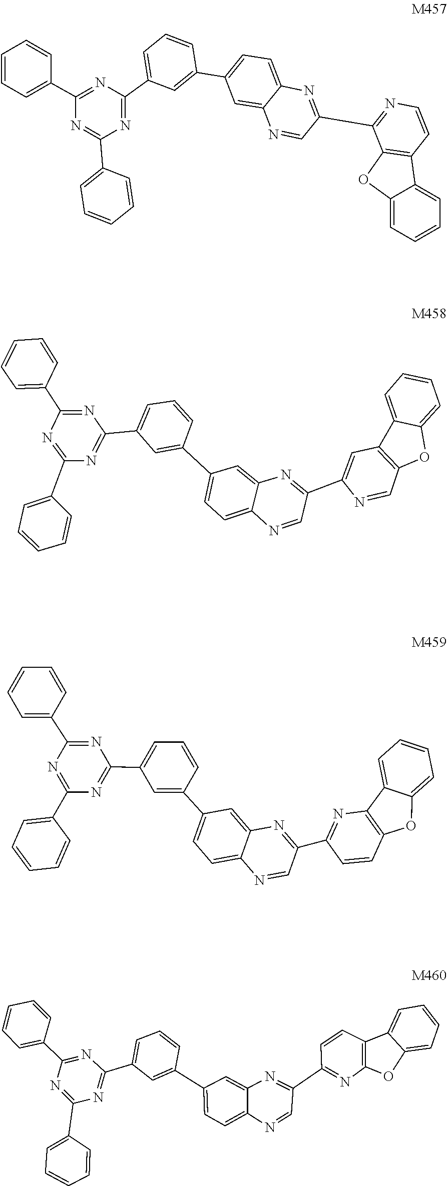

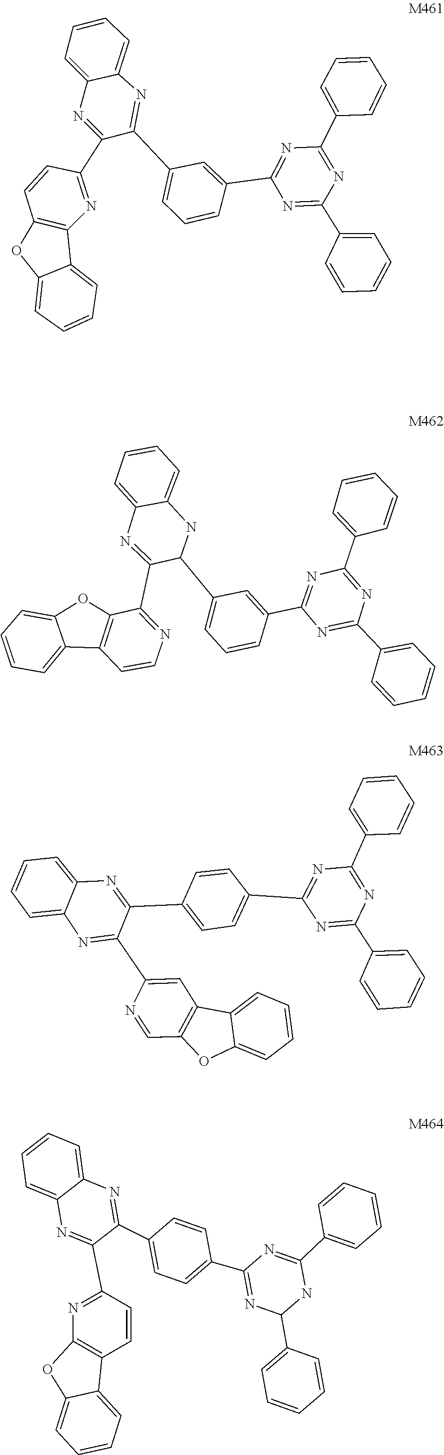 [SMILES: c1ccc(-c2nc(-c3ccccc3)nc(-c3cccc(-c4nc5ccccc5nc4-c4ccc5oc6ccccc6c5n4)c3)n2)cc1 ; c1ccc(-c2nc(-c3ccccc3)nc(-c3cccc(C4Nc5ccccc5N=C4c4nccc5c4oc4ccccc45)c3)n2)cc1 ; c1ccc(-c2nc(-c3ccccc3)nc(-c3ccc(-c4nc5ccccc5nc4-c4cc5c(cn4)oc4ccccc45)cc3)n2)cc1 ; c1ccc(C2=NC(c3ccc(-c4nc5ccccc5nc4-c4ccc5c(n4)oc4ccccc45)cc3)=NC(c3ccccc3)N2)cc1]

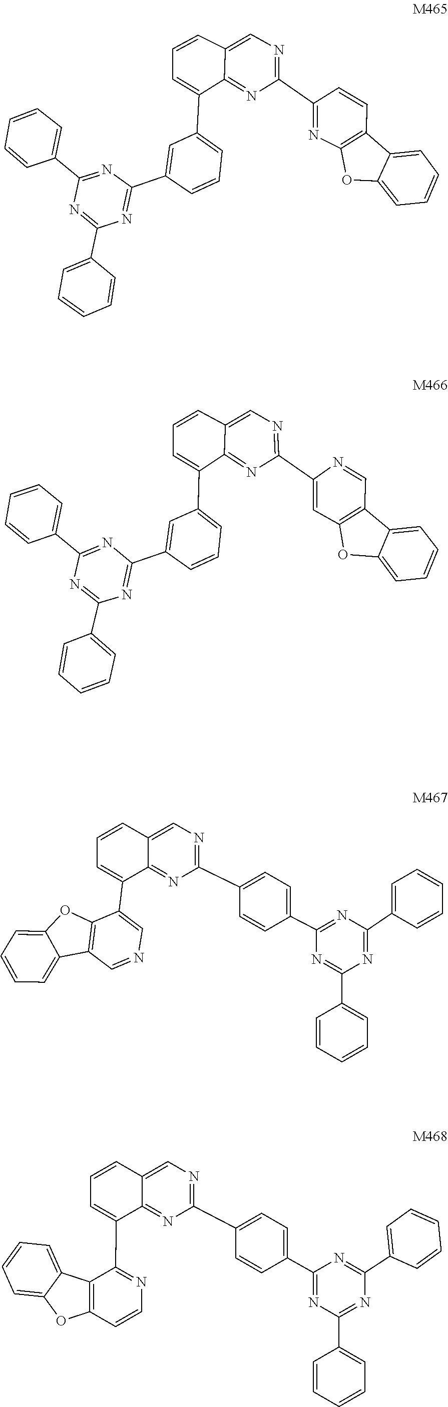 [SMILES: c1ccc(-c2nc(-c3ccccc3)nc(-c3cccc(-c4cccc5cnc(-c6ccc7c(n6)oc6ccccc67)nc45)c3)n2)cc1 ; c1ccc(-c2nc(-c3ccccc3)nc(-c3cccc(-c4cccc5cnc(-c6cc7oc8ccccc8c7cn6)nc45)c3)n2)cc1 ; c1ccc(-c2nc(-c3ccccc3)nc(-c3ccc(-c4ncc5cccc(-c6cncc7c6oc6ccccc67)c5n4)cc3)n2)cc1 ; c1ccc(-c2nc(-c3ccccc3)nc(-c3ccc(-c4ncc5cccc(-c6nccc7oc8ccccc8c67)c5n4)cc3)n2)cc1]

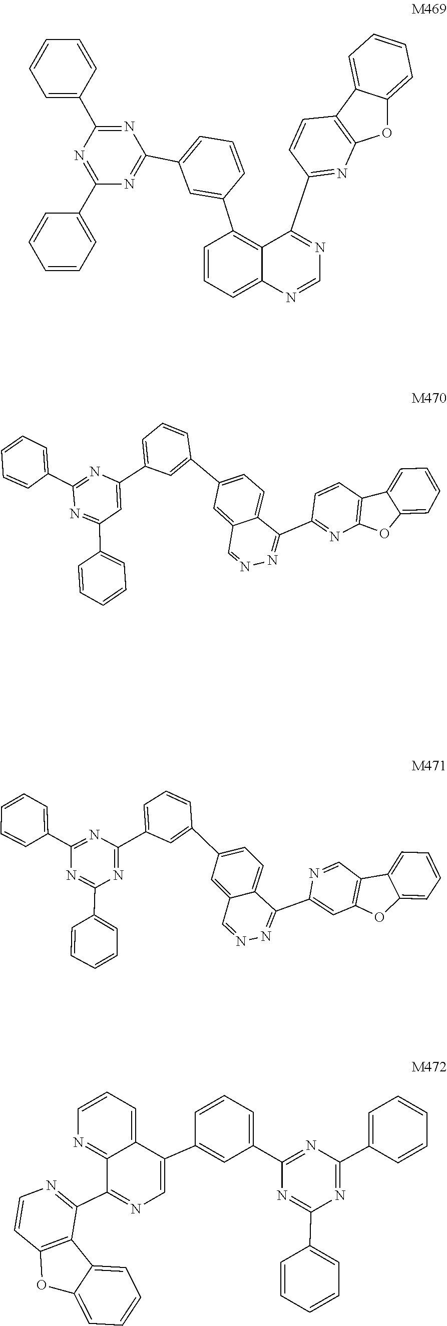 [SMILES: c1ccc(-c2nc(-c3ccccc3)nc(-c3cccc(-c4cccc5ncnc(-c6ccc7c(n6)oc6ccccc67)c45)c3)n2)cc1 ; c1ccc(-c2cc(-c3cccc(-c4ccc5c(-c6ccc7c(n6)oc6ccccc67)nncc5c4)c3)nc(-c3ccccc3)n2)cc1 ; c1ccc(-c2nc(-c3ccccc3)nc(-c3cccc(-c4ccc5c(-c6cc7oc8ccccc8c7cn6)nncc5c4)c3)n2)cc1 ; c1ccc(-c2nc(-c3ccccc3)nc(-c3cccc(-c4cnc(-c5nccc6oc7ccccc7c56)c5ncccc45)c3)n2)cc1]

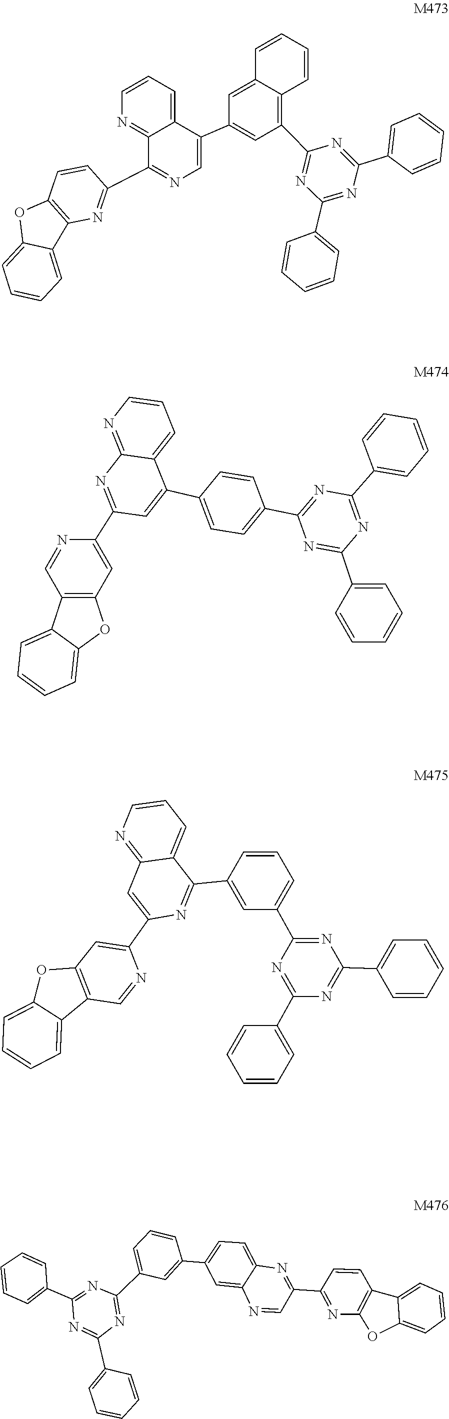 [SMILES: c1ccc(-c2nc(-c3ccccc3)nc(-c3cc(-c4cnc(-c5ccc6oc7ccccc7c6n5)c5ncccc45)cc4ccccc34)n2)cc1 ; c1ccc(-c2nc(-c3ccccc3)nc(-c3ccc(-c4cc(-c5cc6oc7ccccc7c6cn5)nc5ncccc45)cc3)n2)cc1 ; c1ccc(-c2nc(-c3ccccc3)nc(-c3cccc(-c4nc(-c5cc6oc7ccccc7c6cn5)cc5ncccc45)c3)n2)cc1 ; c1ccc(-c2nc(-c3ccccc3)nc(-c3cccc(-c4ccc5nc(-c6ccc7c(n6)oc6ccccc67)cnc5c4)c3)n2)cc1]

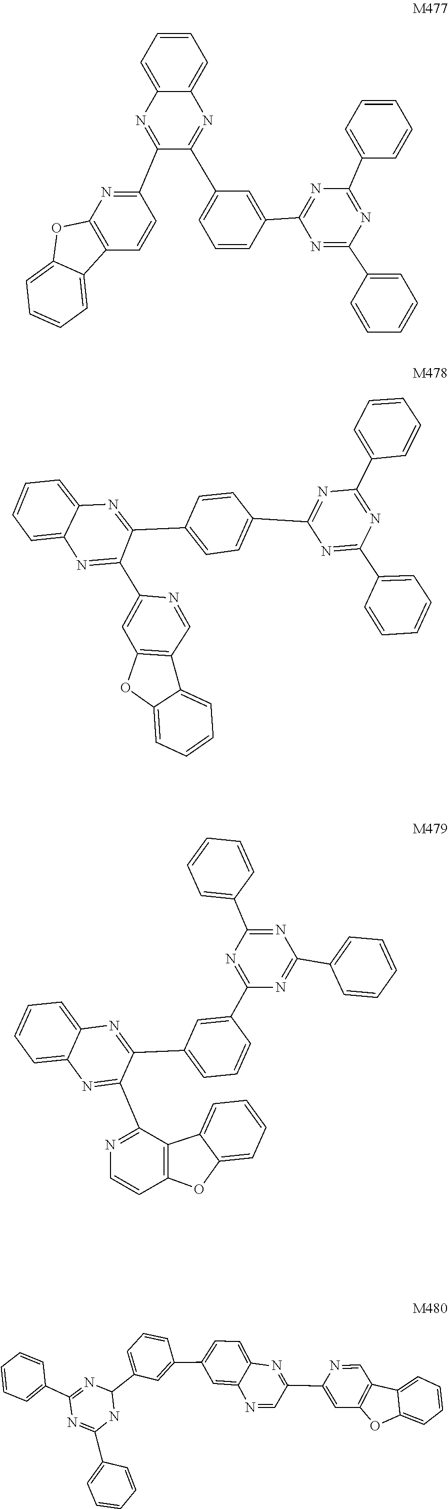 [SMILES: c1ccc(-c2nc(-c3ccccc3)nc(-c3cccc(-c4nc5ccccc5nc4-c4ccc5c(n4)oc4ccccc45)c3)n2)cc1 ; c1ccc(-c2nc(-c3ccccc3)nc(-c3ccc(-c4nc5ccccc5nc4-c4cc5oc6ccccc6c5cn4)cc3)n2)cc1 ; c1ccc(-c2nc(-c3ccccc3)nc(-c3cccc(-c4nc5ccccc5nc4-c4nccc5oc6ccccc6c45)c3)n2)cc1 ; c1ccc(C2=NC(c3cccc(-c4ccc5nc(-c6cc7oc8ccccc8c7cn6)cnc5c4)c3)NC(c3ccccc3)=N2)cc1]

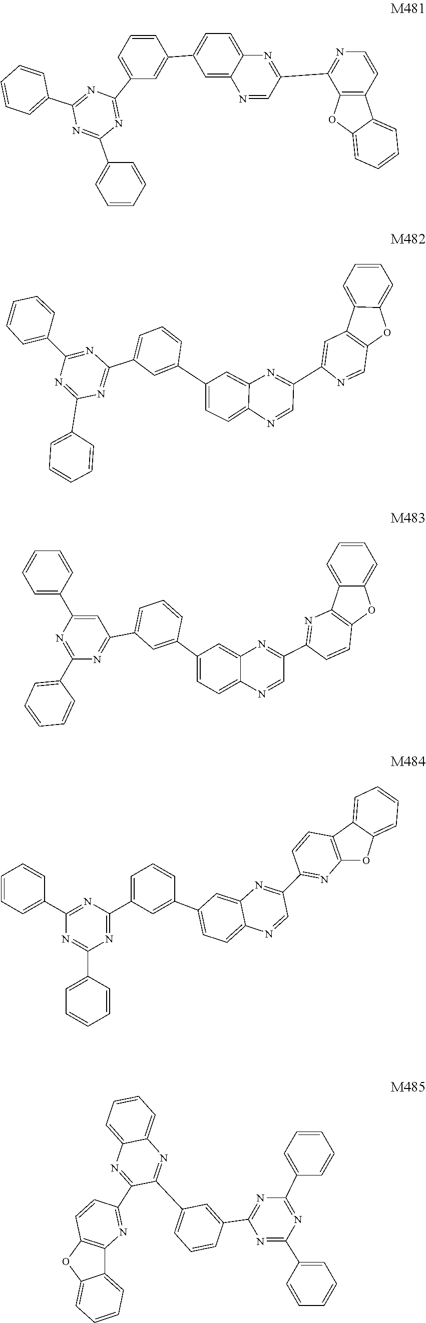 [SMILES: c1ccc(-c2nc(-c3ccccc3)nc(-c3cccc(-c4ccc5nc(-c6nccc7c6oc6ccccc67)cnc5c4)c3)n2)cc1 ; c1ccc(-c2nc(-c3ccccc3)nc(-c3cccc(-c4ccc5ncc(-c6cc7c(cn6)oc6ccccc67)nc5c4)c3)n2)cc1 ; c1ccc(-c2cc(-c3cccc(-c4ccc5ncc(-c6ccc7oc8ccccc8c7n6)nc5c4)c3)nc(-c3ccccc3)n2)cc1 ; c1ccc(-c2nc(-c3ccccc3)nc(-c3cccc(-c4ccc5ncc(-c6ccc7c(n6)oc6ccccc67)nc5c4)c3)n2)cc1 ; c1ccc(-c2nc(-c3ccccc3)nc(-c3cccc(-c4nc5ccccc5nc4-c4ccc5oc6ccccc6c5n4)c3)n2)cc1]

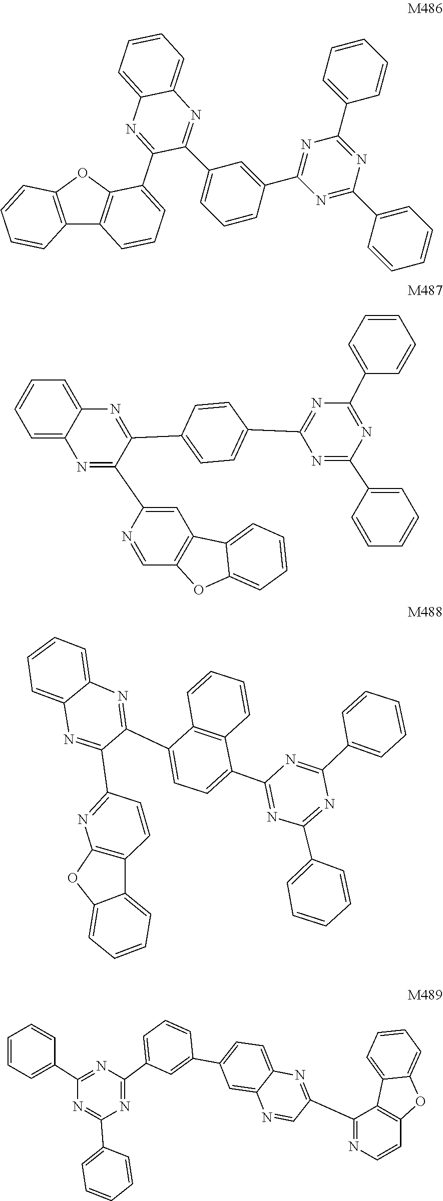

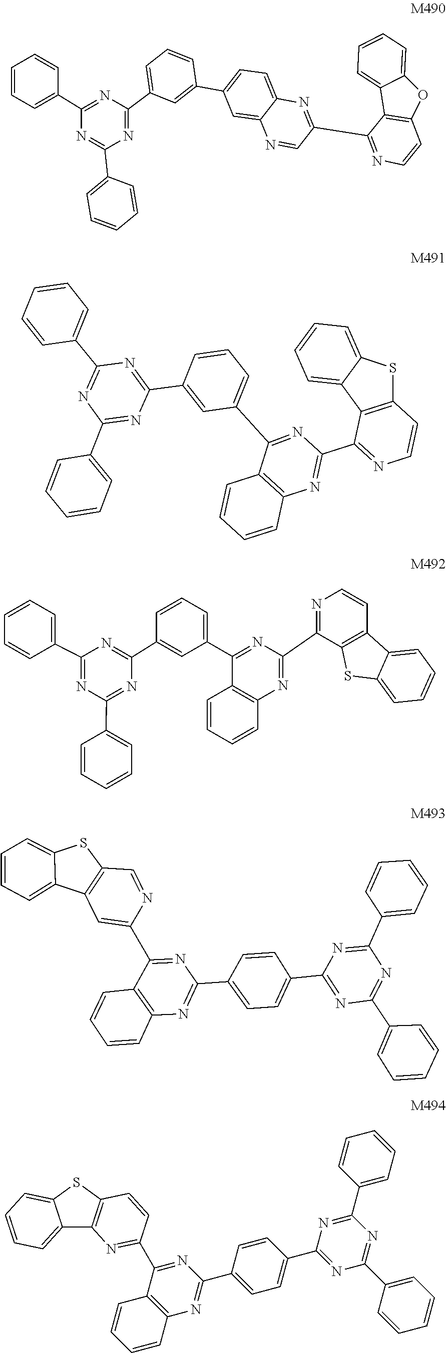 [SMILES: c1ccc(-c2nc(-c3ccccc3)nc(-c3cccc(-c4ccc5nc(-c6nccc7oc8ccccc8c67)cnc5c4)c3)n2)cc1 ; c1ccc(-c2nc(-c3ccccc3)nc(-c3cccc(-c4nc(-c5nccc6sc7ccccc7c56)nc5ccccc45)c3)n2)cc1 ; c1ccc(-c2nc(-c3ccccc3)nc(-c3cccc(-c4nc(-c5nccc6c5sc5ccccc56)nc5ccccc45)c3)n2)cc1 ; c1ccc(-c2nc(-c3ccccc3)nc(-c3ccc(-c4nc(-c5cc6c(cn5)sc5ccccc56)c5ccccc5n4)cc3)n2)cc1 ; c1ccc(-c2nc(-c3ccccc3)nc(-c3ccc(-c4nc(-c5ccc6sc7ccccc7c6n5)c5ccccc5n4)cc3)n2)cc1]

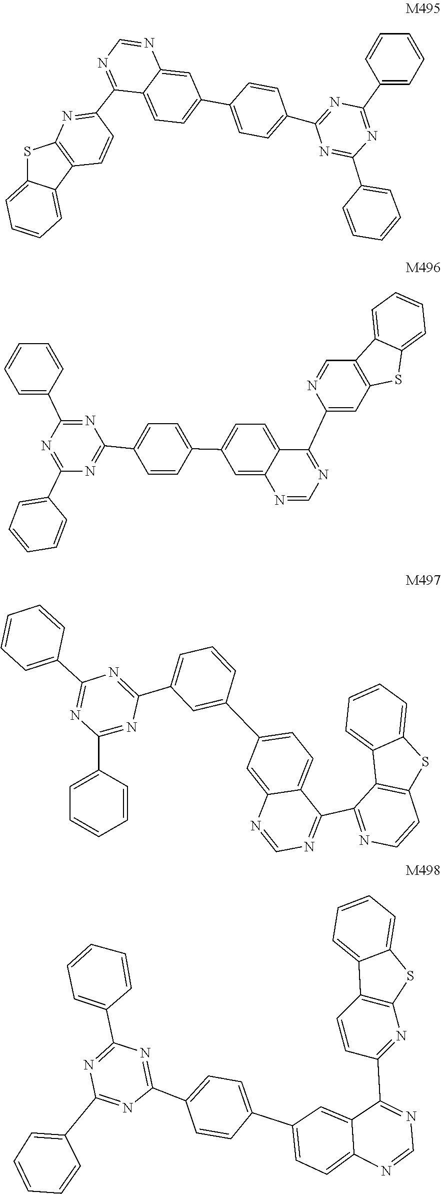 [SMILES: c1ccc(-c2nc(-c3ccccc3)nc(-c3ccc(-c4ccc5c(-c6ccc7c(n6)sc6ccccc67)ncnc5c4)cc3)n2)cc1 ; c1ccc(-c2nc(-c3ccccc3)nc(-c3ccc(-c4ccc5c(-c6cc7sc8ccccc8c7cn6)ncnc5c4)cc3)n2)cc1 ; c1ccc(-c2nc(-c3ccccc3)nc(-c3cccc(-c4ccc5c(-c6nccc7sc8ccccc8c67)ncnc5c4)c3)n2)cc1 ; c1ccc(-c2nc(-c3ccccc3)nc(-c3ccc(-c4ccc5ncnc(-c6ccc7c(n6)sc6ccccc67)c5c4)cc3)n2)cc1]

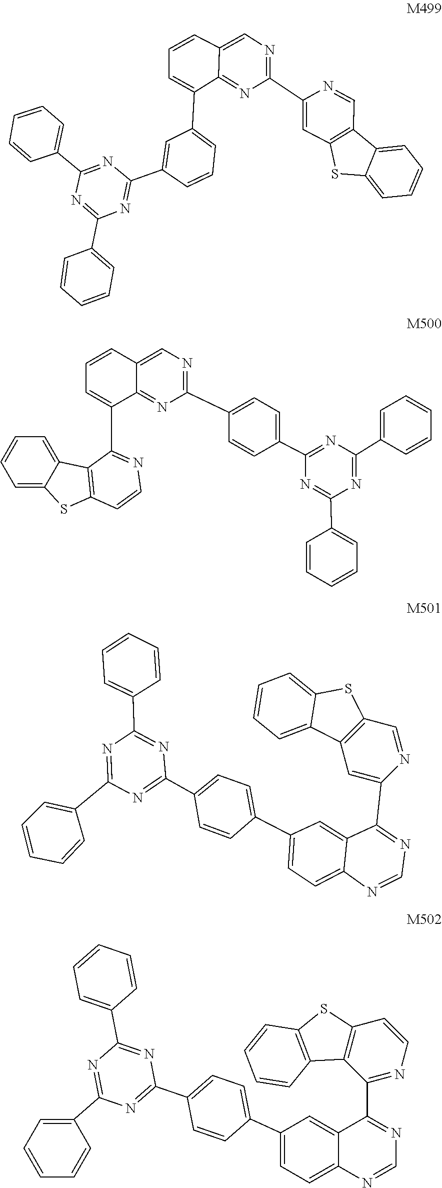 [SMILES: c1ccc(-c2nc(-c3ccccc3)nc(-c3cccc(-c4cccc5cnc(-c6cc7sc8ccccc8c7cn6)nc45)c3)n2)cc1 ; c1ccc(-c2nc(-c3ccccc3)nc(-c3ccc(-c4ncc5cccc(-c6nccc7sc8ccccc8c67)c5n4)cc3)n2)cc1 ; c1ccc(-c2nc(-c3ccccc3)nc(-c3ccc(-c4ccc5ncnc(-c6cc7c(cn6)sc6ccccc67)c5c4)cc3)n2)cc1 ; c1ccc(-c2nc(-c3ccccc3)nc(-c3ccc(-c4ccc5ncnc(-c6nccc7sc8ccccc8c67)c5c4)cc3)n2)cc1]

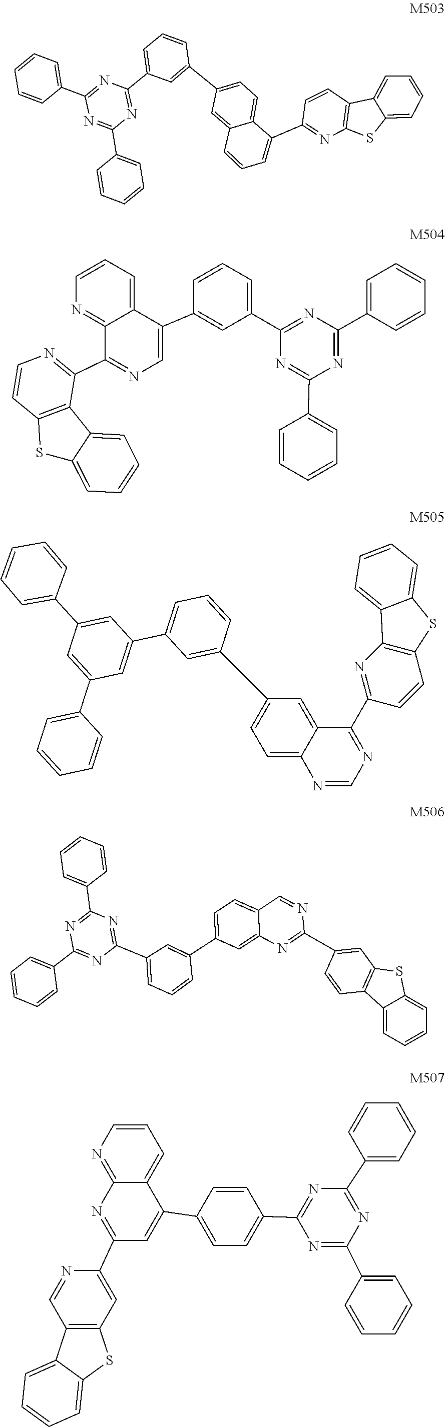 [SMILES: c1ccc(-c2nc(-c3ccccc3)nc(-c3cccc(-c4ccc5c(-c6ccc7c(n6)sc6ccccc67)cccc5c4)c3)n2)cc1 ; c1ccc(-c2nc(-c3ccccc3)nc(-c3cccc(-c4cnc(-c5nccc6sc7ccccc7c56)c5ncccc45)c3)n2)cc1 ; c1ccc(-c2cc(-c3ccccc3)cc(-c3cccc(Cc4ccc5ncnc(-c6ccc7sc8ccccc8c7n6)c5c4)c3)c2)cc1 ; c1ccc(-c2nc(-c3ccccc3)nc(-c3cccc(-c4ccc5cnc(-c6ccc7c(c6)sc6ccccc67)nc5c4)c3)n2)cc1 ; c1ccc(-c2nc(-c3ccccc3)nc(-c3ccc(-c4cc(-c5cc6sc7ccccc7c6cn5)nc5ncccc45)cc3)n2)cc1]

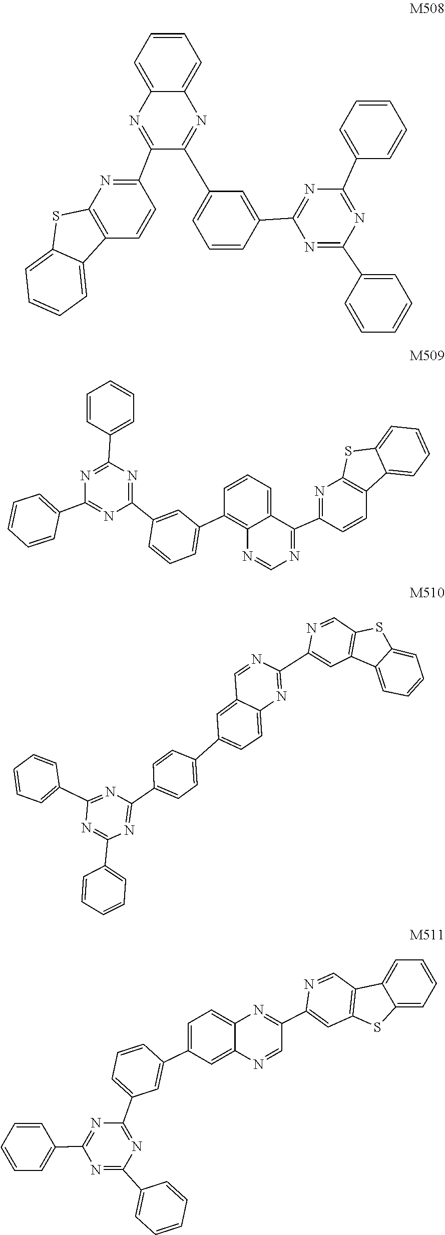 [SMILES: c1ccc(-c2nc(-c3ccccc3)nc(-c3cccc(-c4nc5ccccc5nc4-c4ccc5c(n4)sc4ccccc45)c3)n2)cc1 ; c1ccc(-c2nc(-c3ccccc3)nc(-c3cccc(-c4cccc5c(-c6ccc7c(n6)sc6ccccc67)ncnc45)c3)n2)cc1 ; c1ccc(-c2nc(-c3ccccc3)nc(-c3ccc(-c4ccc5nc(-c6cc7c(cn6)sc6ccccc67)ncc5c4)cc3)n2)cc1 ; c1ccc(-c2nc(-c3ccccc3)nc(-c3cccc(-c4ccc5nc(-c6cc7sc8ccccc8c7cn6)cnc5c4)c3)n2)cc1]

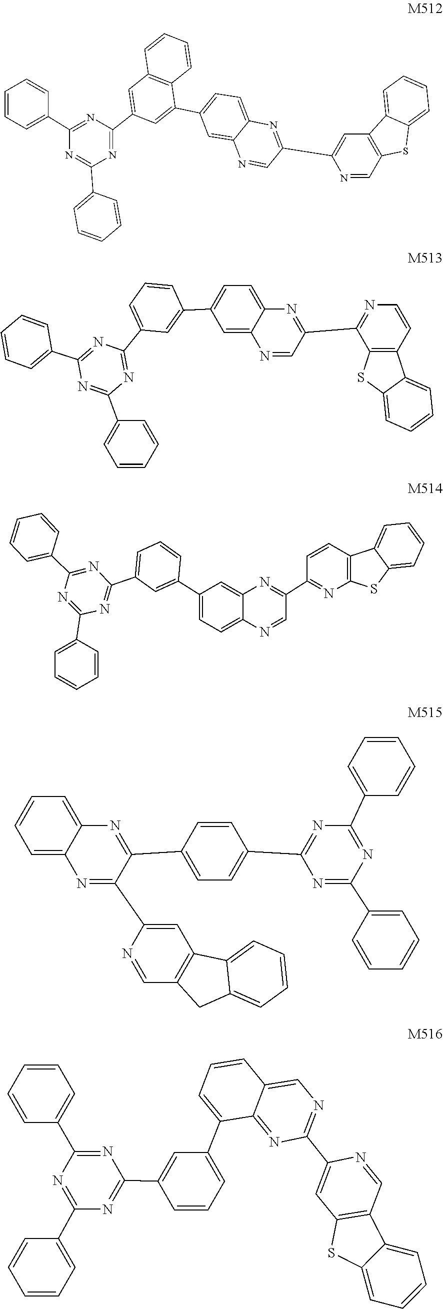 [SMILES: c1ccc(-c2nc(-c3ccccc3)nc(-c3cc(-c4ccc5nc(-c6cc7c(cn6)sc6ccccc67)cnc5c4)c4ccccc4c3)n2)cc1 ; c1ccc(-c2nc(-c3ccccc3)nc(-c3cccc(-c4ccc5nc(-c6nccc7c6sc6ccccc67)cnc5c4)c3)n2)cc1 ; c1ccc(-c2nc(-c3ccccc3)nc(-c3cccc(-c4ccc5ncc(-c6ccc7c(n6)sc6ccccc67)nc5c4)c3)n2)cc1 ; c1ccc(-c2nc(-c3ccccc3)nc(-c3ccc(-c4nc5ccccc5nc4-c4cc5c(cn4)Cc4ccccc4-5)cc3)n2)cc1 ; c1ccc(-c2nc(-c3ccccc3)nc(-c3cccc(-c4cccc5cnc(-c6cc7sc8ccccc8c7cn6)nc45)c3)n2)cc1]

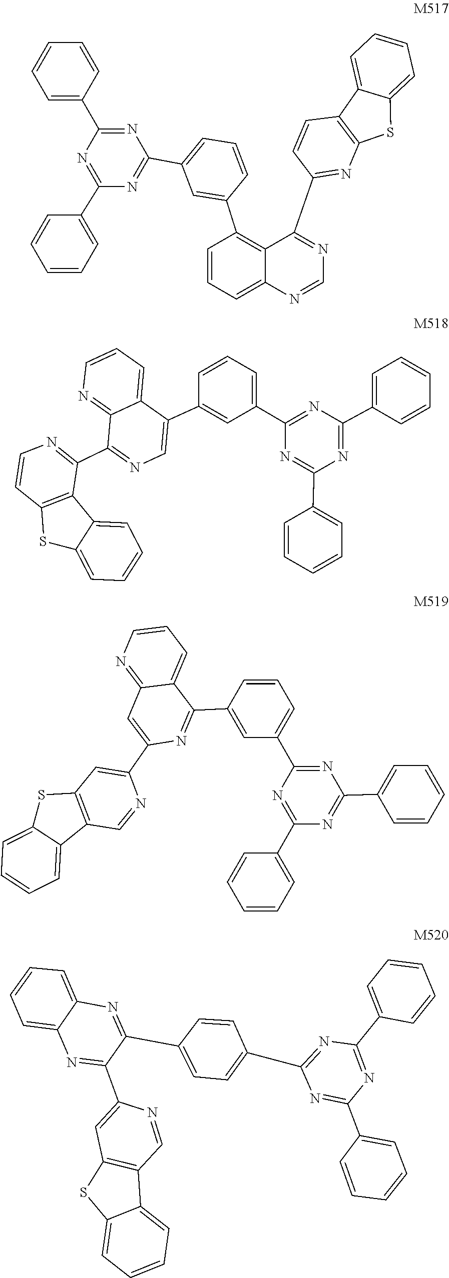 [SMILES: c1ccc(-c2nc(-c3ccccc3)nc(-c3cccc(-c4cccc5ncnc(-c6ccc7c(n6)sc6ccccc67)c45)c3)n2)cc1 ; c1ccc(-c2nc(-c3ccccc3)nc(-c3cccc(-c4cnc(-c5nccc6sc7ccccc7c56)c5ncccc45)c3)n2)cc1 ; c1ccc(-c2nc(-c3ccccc3)nc(-c3cccc(-c4nc(-c5cc6sc7ccccc7c6cn5)cc5ncccc45)c3)n2)cc1 ; c1ccc(-c2nc(-c3ccccc3)nc(-c3ccc(-c4nc5ccccc5nc4-c4cc5sc6ccccc6c5cn4)cc3)n2)cc1]

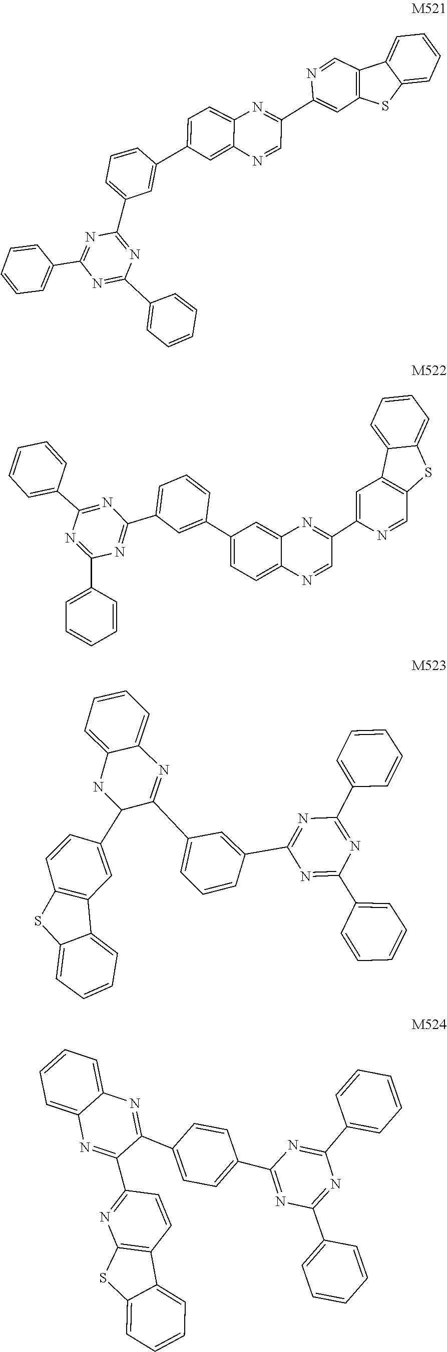 [SMILES: c1ccc(-c2nc(-c3ccccc3)nc(-c3cccc(-c4ccc5nc(-c6cc7sc8ccccc8c7cn6)cnc5c4)c3)n2)cc1 ; c1ccc(-c2nc(-c3ccccc3)nc(-c3cccc(-c4ccc5ncc(-c6cc7c(cn6)sc6ccccc67)nc5c4)c3)n2)cc1 ; c1ccc(-c2nc(-c3ccccc3)nc(-c3cccc(C4=Nc5ccccc5NC4c4ccc5sc6ccccc6c5c4)c3)n2)cc1 ; c1ccc(-c2nc(-c3ccccc3)nc(-c3ccc(-c4nc5ccccc5nc4-c4ccc5c(n4)sc4ccccc45)cc3)n2)cc1]

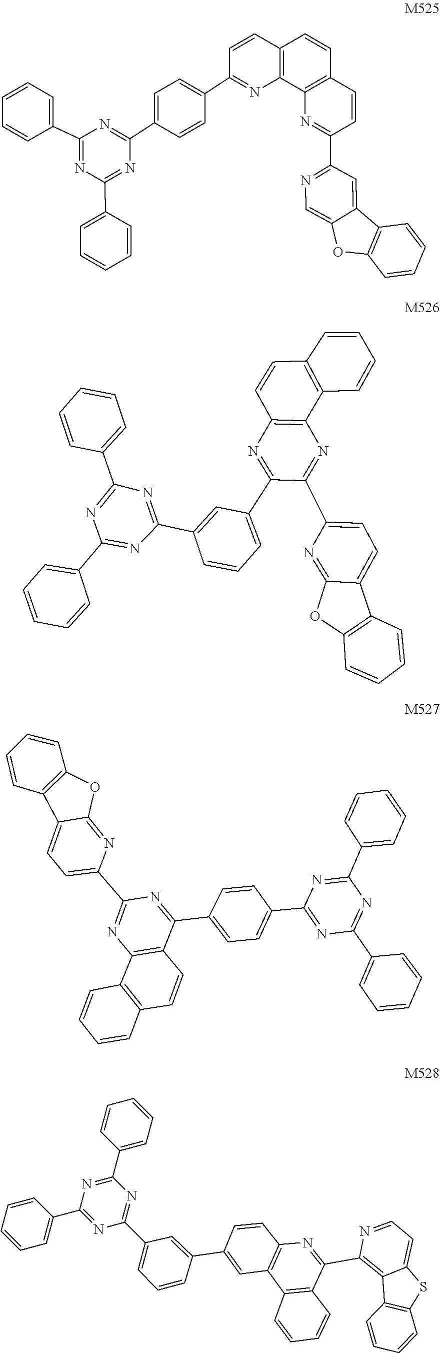 [SMILES: c1ccc(-c2nc(-c3ccccc3)nc(-c3ccc(-c4ccc5ccc6ccc(-c7cc8c(cn7)oc7ccccc78)nc6c5n4)cc3)n2)cc1 ; c1ccc(-c2nc(-c3ccccc3)nc(-c3cccc(-c4nc5ccc6ccccc6c5nc4-c4ccc5c(n4)oc4ccccc45)c3)n2)cc1 ; c1ccc(-c2nc(-c3ccccc3)nc(-c3ccc(-c4nc(-c5ccc6c(n5)OCc5ccccc5-6)nc5c4ccc4ccccc45)cc3)n2)cc1 ; c1ccc(-c2nc(-c3ccccc3)nc(-c3cccc(-c4ccc5nc(-c6nccc7sc8ccccc8c67)c6ccccc6c5c4)c3)n2)cc1]

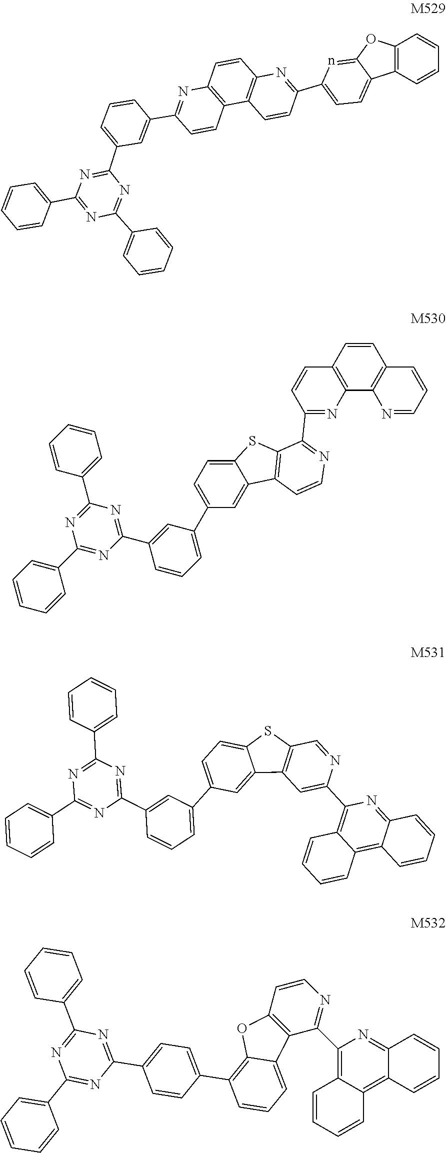

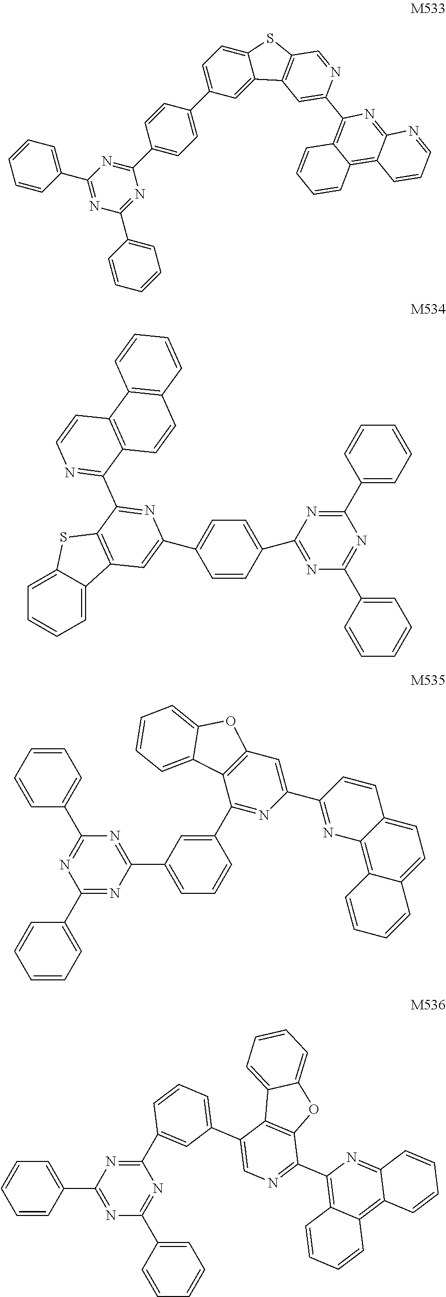

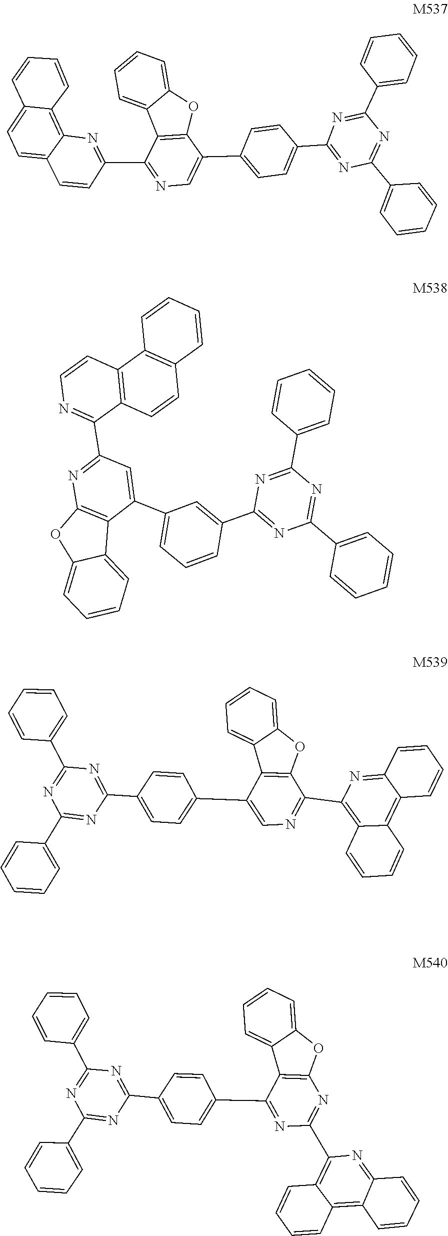 [SMILES: c1ccc(-c2nc(-c3ccccc3)nc(-c3ccc(-c4cnc(-c5ccc6ccc7ccccc7c6n5)c5c4oc4ccccc45)cc3)n2)cc1 ; c1ccc(-c2nc(-c3ccccc3)nc(-c3cccc(-c4cc(-c5nccc6c5ccc5ccccc56)nc5oc6ccccc6c45)c3)n2)cc1 ; c1ccc(-c2nc(-c3ccccc3)nc(-c3ccc(-c4cnc(-c5nc6ccccc6c6ccccc56)c5oc6ccccc6c45)cc3)n2)cc1 ; c1ccc(-c2nc(-c3ccccc3)nc(-c3ccc(-c4nc(-c5nc6ccccc6c6ccccc56)nc5oc6ccccc6c45)cc3)n2)cc1]

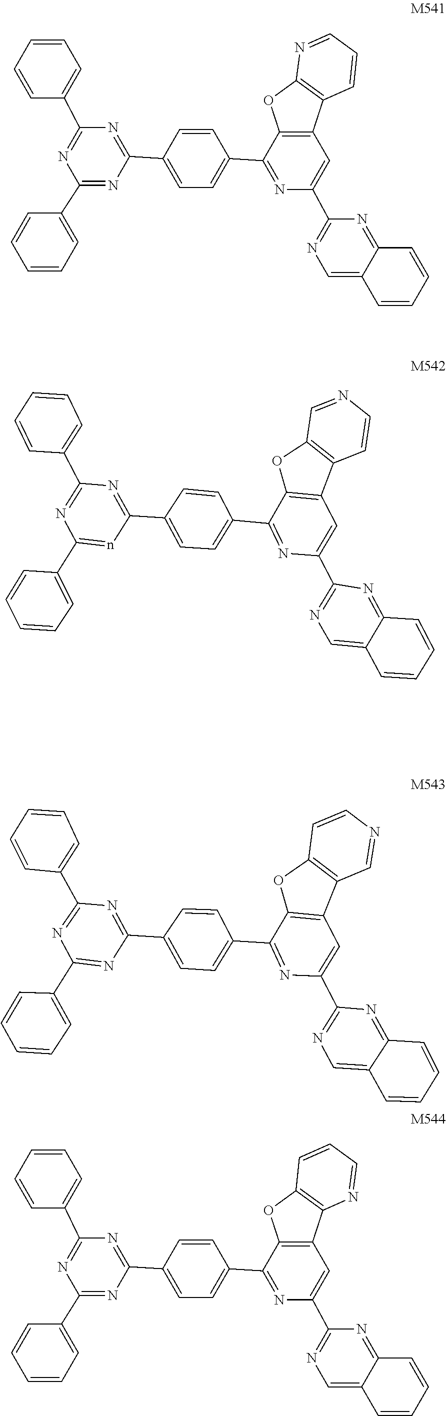

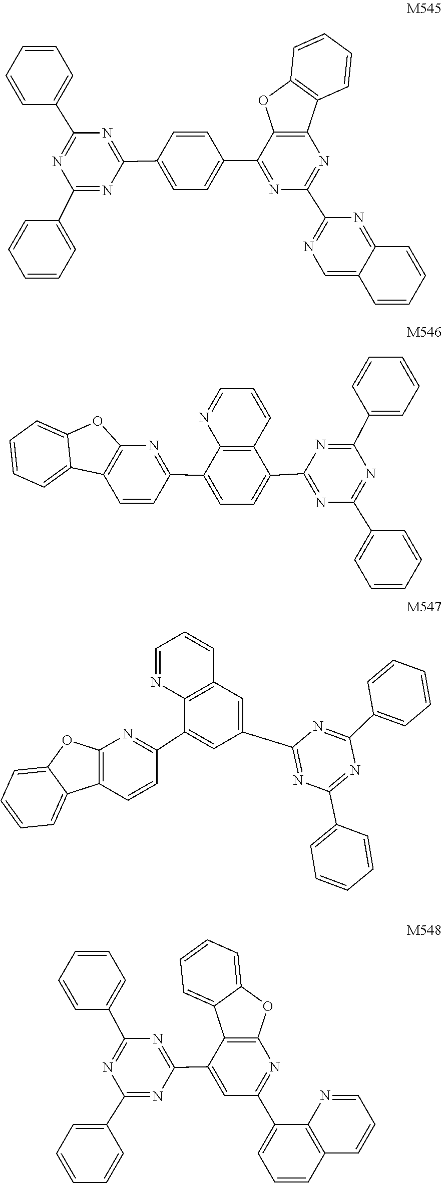 [SMILES: c1ccc(-c2nc(-c3ccccc3)nc(-c3ccc(-c4nc(-c5ncc6ccccc6n5)nc5c4oc4ccccc45)cc3)n2)cc1 ; c1ccc(-c2nc(-c3ccccc3)nc(-c3ccc(-c4ccc5c(n4)oc4ccccc45)c4ncccc34)n2)cc1 ; c1ccc(-c2nc(-c3ccccc3)nc(-c3cc(-c4ccc5c(n4)oc4ccccc45)c4ncccc4c3)n2)cc1 ; c1ccc(-c2nc(-c3ccccc3)nc(-c3cc(-c4cccc5cccnc45)nc4oc5ccccc5c34)n2)cc1]

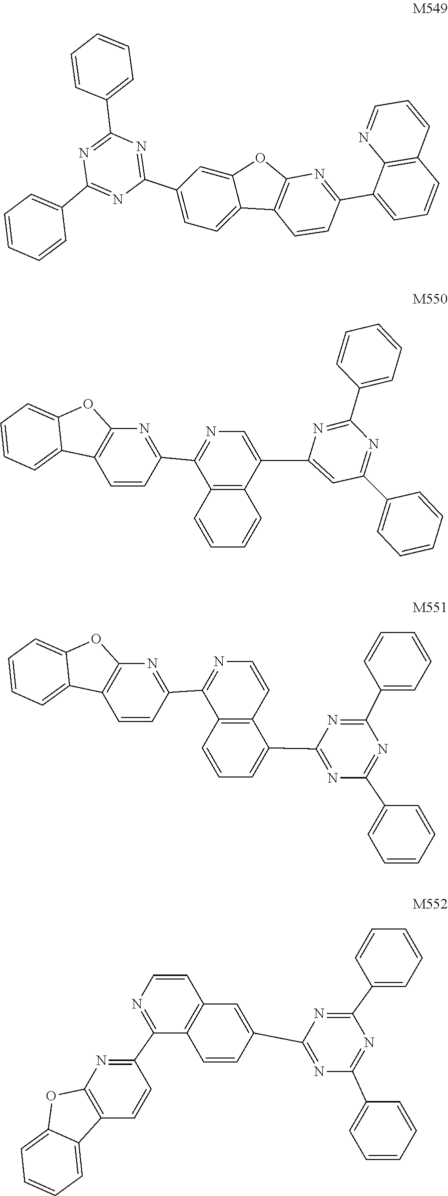 [SMILES: c1ccc(-c2nc(-c3ccccc3)nc(-c3ccc4c(c3)oc3nc(-c5cccc6cccnc56)ccc34)n2)cc1 ; c1ccc(-c2cc(-c3cnc(-c4ccc5c(n4)oc4ccccc45)c4ccccc34)nc(-c3ccccc3)n2)cc1 ; c1ccc(-c2nc(-c3ccccc3)nc(-c3cccc4c(-c5ccc6c(n5)oc5ccccc56)nccc34)n2)cc1 ; c1ccc(-c2nc(-c3ccccc3)nc(-c3ccc4c(-c5ccc6c(n5)oc5ccccc56)nccc4c3)n2)cc1]

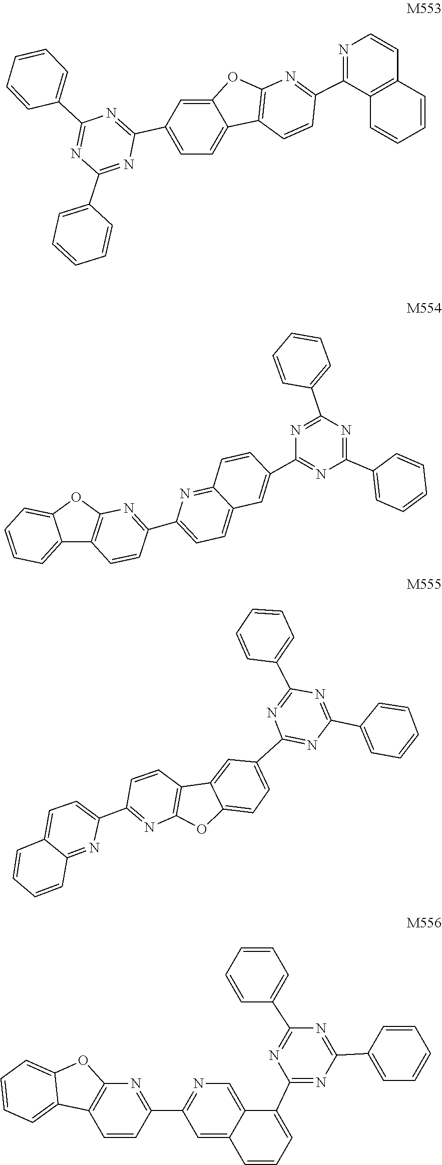 [SMILES: c1ccc(-c2nc(-c3ccccc3)nc(-c3ccc4c(c3)oc3nc(-c5nccc6ccccc56)ccc34)n2)cc1 ; c1ccc(-c2nc(-c3ccccc3)nc(-c3ccc4nc(-c5ccc6c(n5)oc5ccccc56)ccc4c3)n2)cc1 ; c1ccc(-c2nc(-c3ccccc3)nc(-c3ccc4oc5nc(-c6ccc7ccccc7n6)ccc5c4c3)n2)cc1 ; c1ccc(-c2nc(-c3ccccc3)nc(-c3cccc4cc(-c5ccc6c(n5)oc5ccccc56)ncc34)n2)cc1]

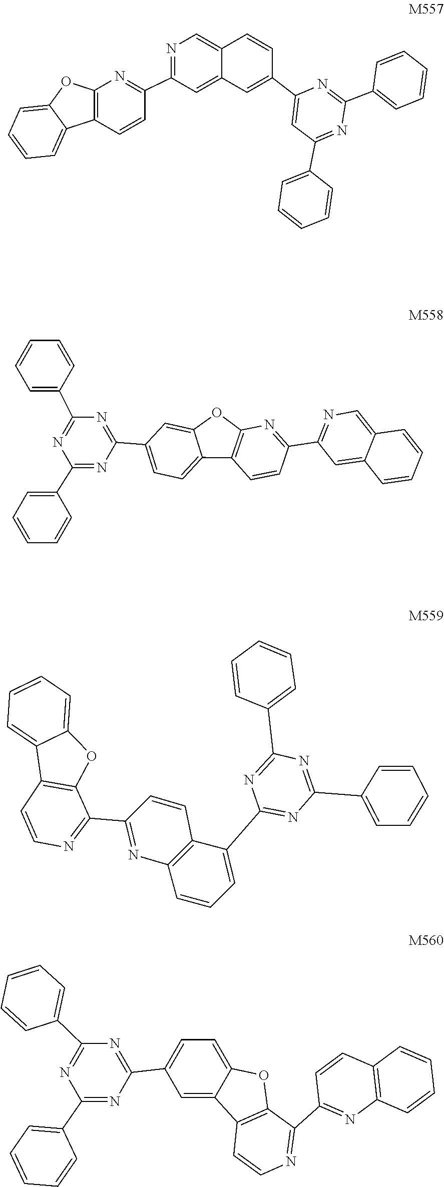 [SMILES: c1ccc(-c2cc(-c3ccc4cnc(-c5ccc6c(n5)oc5ccccc56)cc4c3)nc(-c3ccccc3)n2)cc1 ; c1ccc(-c2nc(-c3ccccc3)nc(-c3ccc4c(c3)oc3nc(-c5cc6ccccc6cn5)ccc34)n2)cc1 ; c1ccc(-c2nc(-c3ccccc3)nc(-c3cccc4nc(-c5nccc6c5oc5ccccc56)ccc34)n2)cc1 ; c1ccc(-c2nc(-c3ccccc3)nc(-c3ccc4oc5c(-c6ccc7ccccc7n6)nccc5c4c3)n2)cc1]

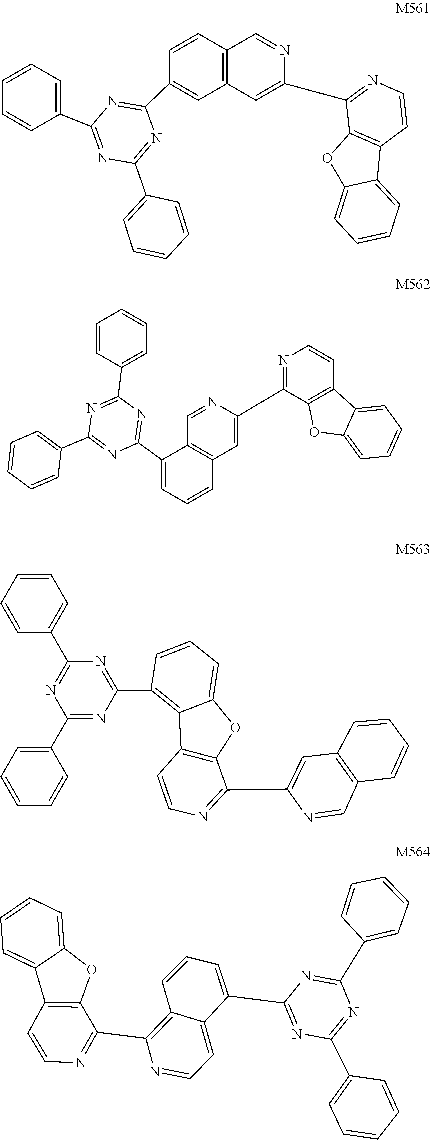 [SMILES: c1ccc(-c2nc(-c3ccccc3)nc(-c3ccc4cnc(-c5nccc6c5oc5ccccc56)cc4c3)n2)cc1 ; c1ccc(-c2nc(-c3ccccc3)nc(-c3cccc4cc(-c5nccc6c5oc5ccccc56)ncc34)n2)cc1 ; c1ccc(-c2nc(-c3ccccc3)nc(-c3cccc4oc5c(-c6cc7ccccc7cn6)nccc5c34)n2)cc1 ; c1ccc(-c2nc(-c3ccccc3)nc(-c3cccc4c(-c5nccc6c5oc5ccccc56)nccc34)n2)cc1]

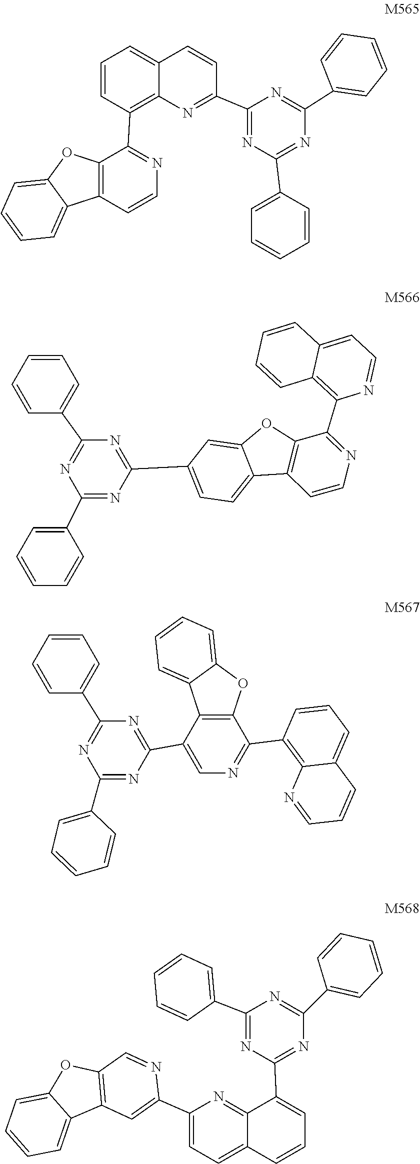 [SMILES: c1ccc(-c2nc(-c3ccccc3)nc(-c3ccc4cccc(-c5nccc6c5oc5ccccc56)c4n3)n2)cc1 ; c1ccc(-c2nc(-c3ccccc3)nc(-c3ccc4c(c3)oc3c(-c5nccc6ccccc56)nccc34)n2)cc1 ; c1ccc(-c2nc(-c3ccccc3)nc(-c3cnc(-c4cccc5cccnc45)c4oc5ccccc5c34)n2)cc1 ; c1ccc(-c2nc(-c3ccccc3)nc(-c3cccc4ccc(-c5cc6c(cn5)oc5ccccc56)nc34)n2)cc1]

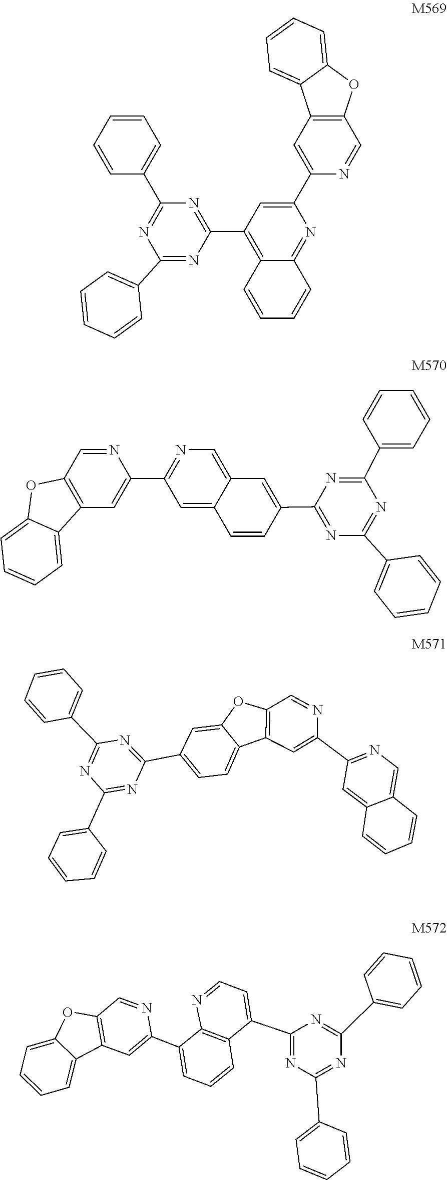 [SMILES: c1ccc(-c2nc(-c3ccccc3)nc(-c3cc(-c4cc5c(cn4)oc4ccccc45)nc4ccccc34)n2)cc1 ; c1ccc(-c2nc(-c3ccccc3)nc(-c3ccc4cc(-c5cc6c(cn5)oc5ccccc56)ncc4c3)n2)cc1 ; c1ccc(-c2nc(-c3ccccc3)nc(-c3ccc4c(c3)oc3cnc(-c5cc6ccccc6cn5)cc34)n2)cc1 ; c1ccc(-c2nc(-c3ccccc3)nc(-c3ccnc4c(-c5cc6c(cn5)oc5ccccc56)cccc34)n2)cc1]

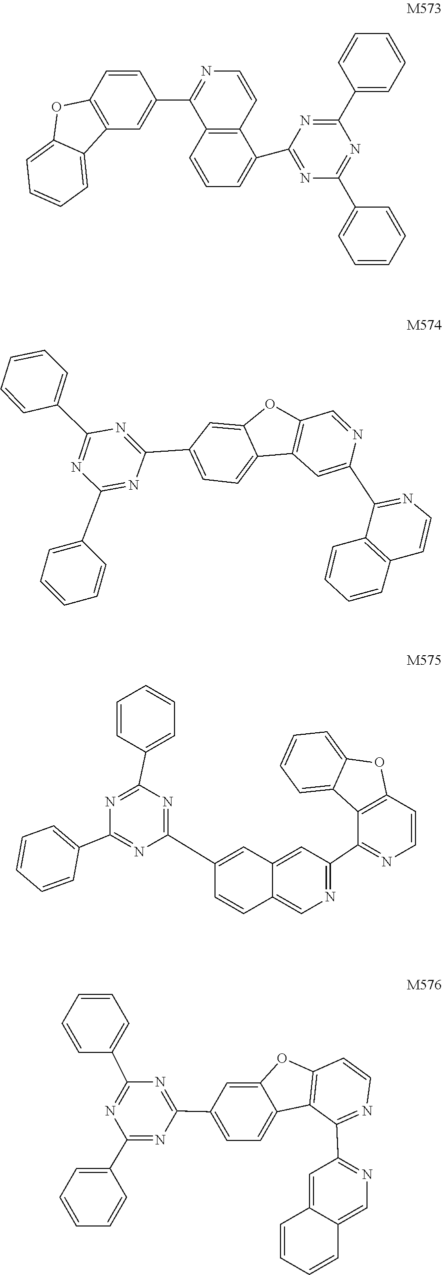 [SMILES: c1ccc(-c2nc(-c3ccccc3)nc(-c3cccc4c(-c5ccc6oc7ccccc7c6c5)nccc34)n2)cc1 ; c1ccc(-c2nc(-c3ccccc3)nc(-c3ccc4c(c3)oc3cnc(-c5nccc6ccccc56)cc34)n2)cc1 ; c1ccc(-c2nc(-c3ccccc3)nc(-c3ccc4cnc(-c5nccc6oc7ccccc7c56)cc4c3)n2)cc1 ; c1ccc(-c2nc(-c3ccccc3)nc(-c3ccc4c(c3)oc3ccnc(-c5cc6ccccc6cn5)c34)n2)cc1]

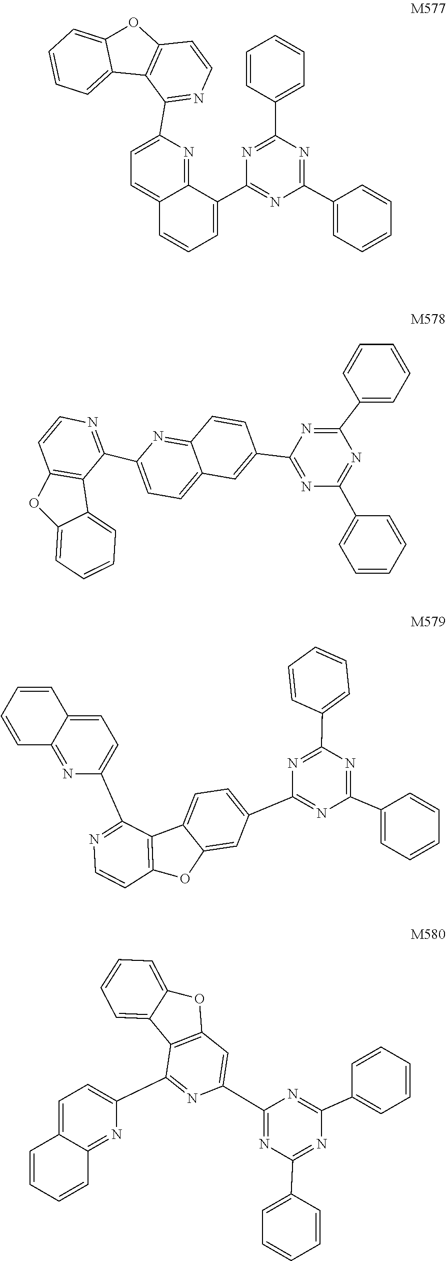 [SMILES: c1ccc(-c2nc(-c3ccccc3)nc(-c3cccc4ccc(-c5nccc6oc7ccccc7c56)nc34)n2)cc1 ; c1ccc(-c2nc(-c3ccccc3)nc(-c3ccc4nc(-c5nccc6oc7ccccc7c56)ccc4c3)n2)cc1 ; c1ccc(-c2nc(-c3ccccc3)nc(-c3ccc4c(c3)oc3ccnc(-c5ccc6ccccc6n5)c34)n2)cc1 ; c1ccc(-c2nc(-c3ccccc3)nc(-c3cc4oc5ccccc5c4c(-c4ccc5ccccc5n4)n3)n2)cc1]

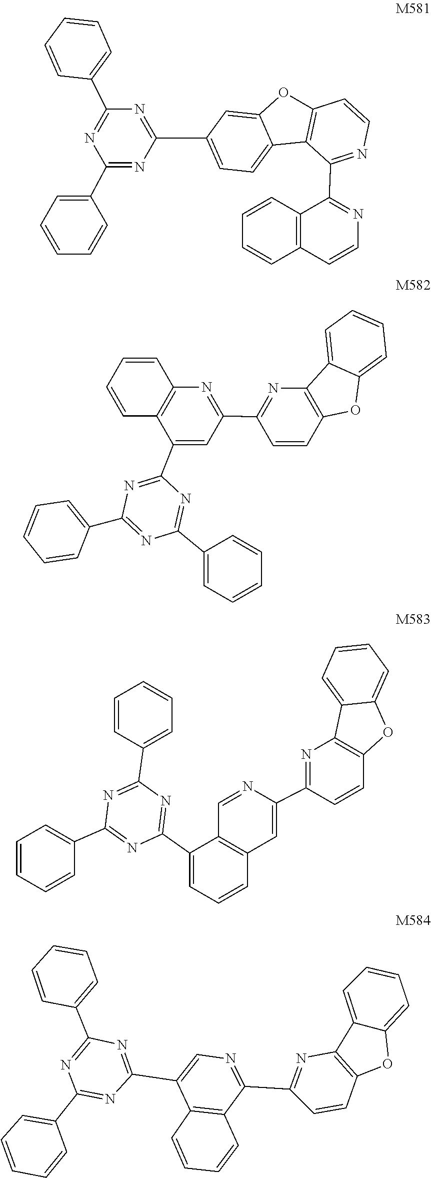 [SMILES: c1ccc(-c2nc(-c3ccccc3)nc(-c3ccc4c(c3)oc3ccnc(-c5nccc6ccccc56)c34)n2)cc1 ; c1ccc(-c2nc(-c3ccccc3)nc(-c3cc(-c4ccc5oc6ccccc6c5n4)nc4ccccc34)n2)cc1 ; c1ccc(-c2nc(-c3ccccc3)nc(-c3cccc4cc(-c5ccc6oc7ccccc7c6n5)ncc34)n2)cc1 ; c1ccc(-c2nc(-c3ccccc3)nc(-c3cnc(-c4ccc5oc6ccccc6c5n4)c4ccccc34)n2)cc1]

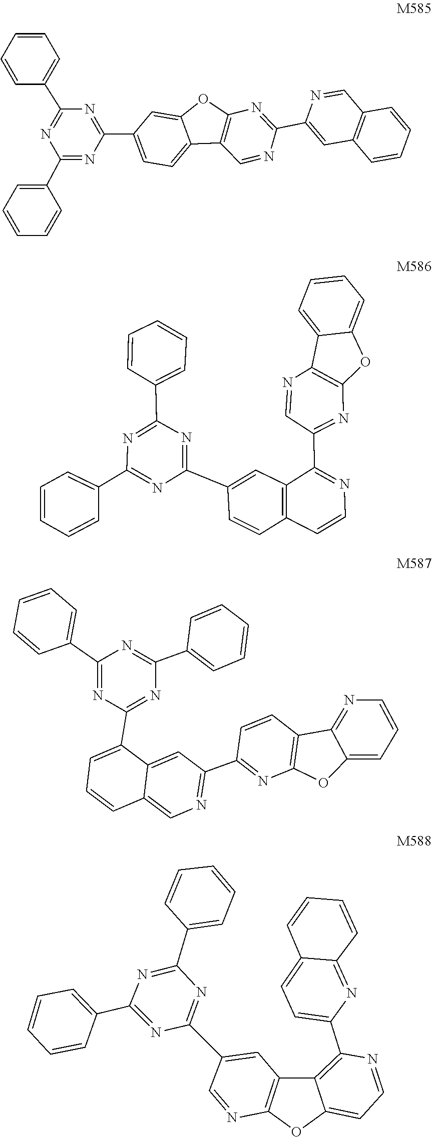 [SMILES: c1ccc(-c2nc(-c3ccccc3)nc(-c3ccc4c(c3)oc3nc(-c5cc6ccccc6cn5)ncc34)n2)cc1 ; c1ccc(-c2nc(-c3ccccc3)nc(-c3ccc4ccnc(-c5cnc6c(n5)oc5ccccc56)c4c3)n2)cc1 ; c1ccc(-c2nc(-c3ccccc3)nc(-c3cccc4cnc(-c5ccc6c(n5)oc5cccnc56)cc34)n2)cc1 ; c1ccc(-c2nc(-c3ccccc3)nc(-c3cnc4oc5ccnc(-c6ccc7ccccc7n6)c5c4c3)n2)cc1]

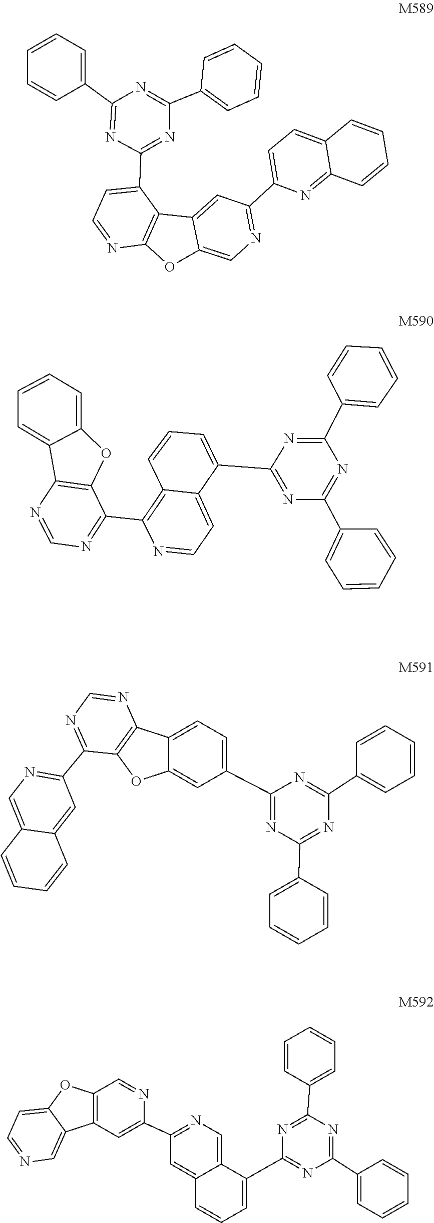 [SMILES: c1ccc(-c2nc(-c3ccccc3)nc(-c3ccnc4oc5cnc(-c6ccc7ccccc7n6)cc5c34)n2)cc1 ; c1ccc(-c2nc(-c3ccccc3)nc(-c3cccc4c(-c5ncnc6c5oc5ccccc56)nccc34)n2)cc1 ; c1ccc(-c2nc(-c3ccccc3)nc(-c3ccc4c(c3)oc3c(-c5cc6ccccc6cn5)ncnc34)n2)cc1 ; c1ccc(-c2nc(-c3ccccc3)nc(-c3cccc4cc(-c5cc6c(cn5)oc5ccncc56)ncc34)n2)cc1]

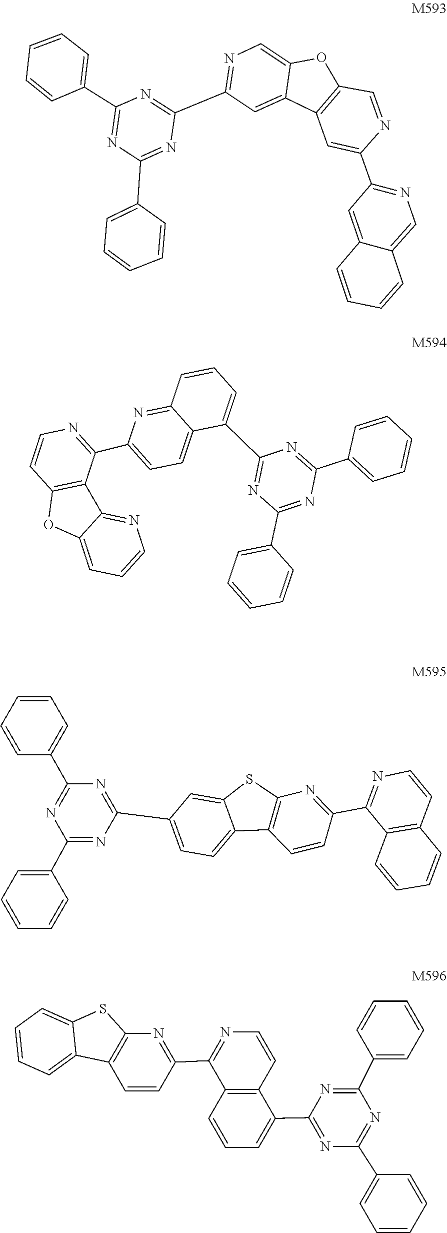 [SMILES: c1ccc(-c2nc(-c3ccccc3)nc(-c3cc4c(cn3)oc3cnc(-c5cc6ccccc6cn5)cc34)n2)cc1 ; c1ccc(-c2nc(-c3ccccc3)nc(-c3cccc4nc(-c5nccc6oc7cccnc7c56)ccc34)n2)cc1 ; c1ccc(-c2nc(-c3ccccc3)nc(-c3ccc4c(c3)sc3nc(-c5nccc6ccccc56)ccc34)n2)cc1 ; c1ccc(-c2nc(-c3ccccc3)nc(-c3cccc4c(-c5ccc6c(n5)sc5ccccc56)nccc34)n2)cc1]

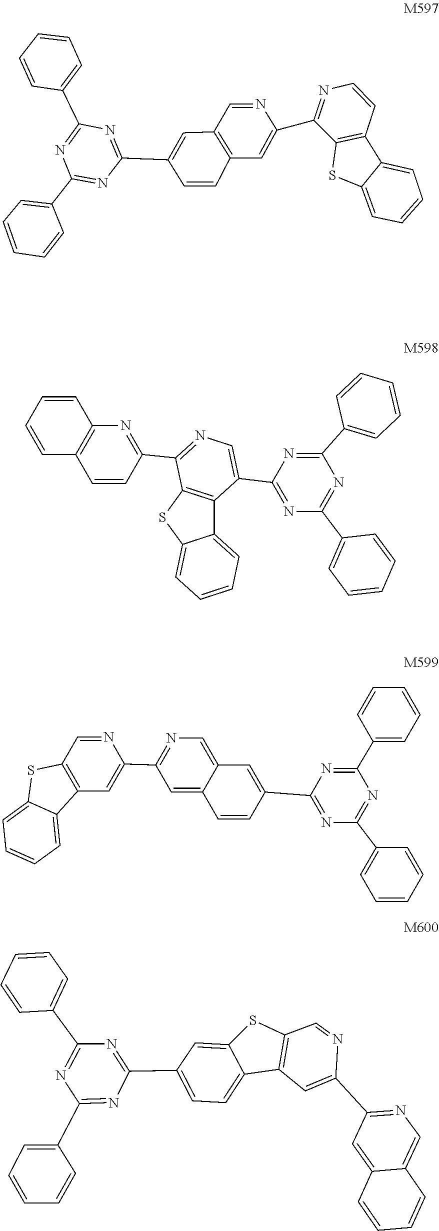 [SMILES: c1ccc(-c2nc(-c3ccccc3)nc(-c3ccc4cc(-c5nccc6c5sc5ccccc56)ncc4c3)n2)cc1 ; c1ccc(-c2nc(-c3ccccc3)nc(-c3cnc(-c4ccc5ccccc5n4)c4sc5ccccc5c34)n2)cc1 ; c1ccc(-c2nc(-c3ccccc3)nc(-c3ccc4cc(-c5cc6c(cn5)sc5ccccc56)ncc4c3)n2)cc1 ; c1ccc(-c2nc(-c3ccccc3)nc(-c3ccc4c(c3)sc3cnc(-c5cc6ccccc6cn5)cc34)n2)cc1]

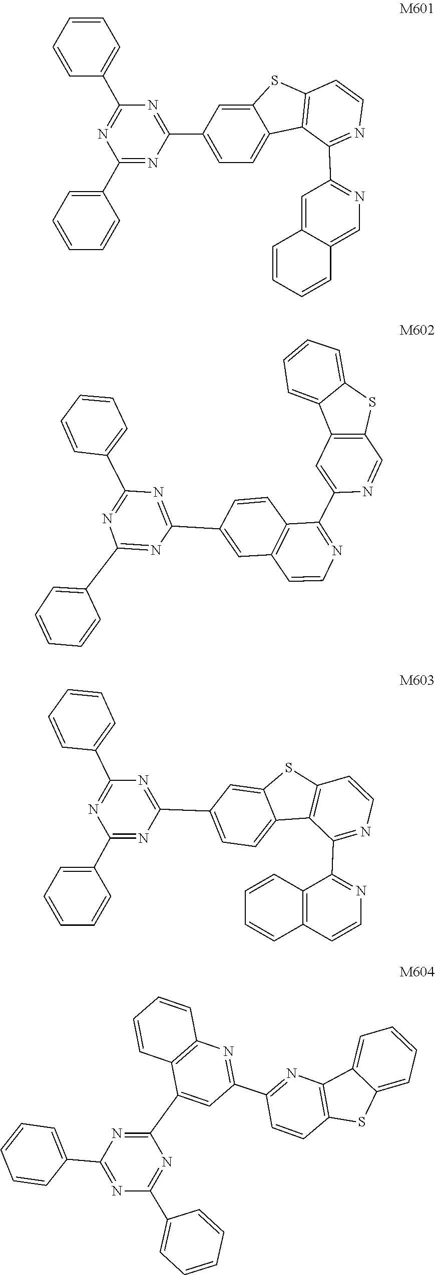

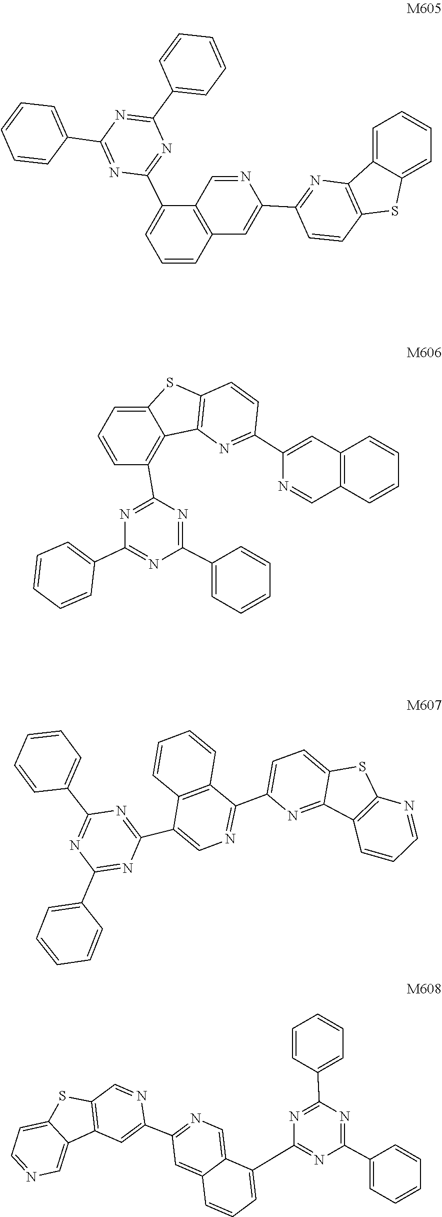 [SMILES: c1ccc(-c2nc(-c3ccccc3)nc(-c3cccc4cc(-c5ccc6sc7ccccc7c6n5)ncc34)n2)cc1 ; c1ccc(-c2nc(-c3ccccc3)nc(-c3cccc4sc5ccc(-c6cc7ccccc7cn6)nc5c34)n2)cc1 ; c1ccc(-c2nc(-c3ccccc3)nc(-c3cnc(-c4ccc5sc6ncccc6c5n4)c4ccccc34)n2)cc1 ; c1ccc(-c2nc(-c3ccccc3)nc(-c3cccc4cc(-c5cc6c(cn5)sc5ccncc56)ncc34)n2)cc1]

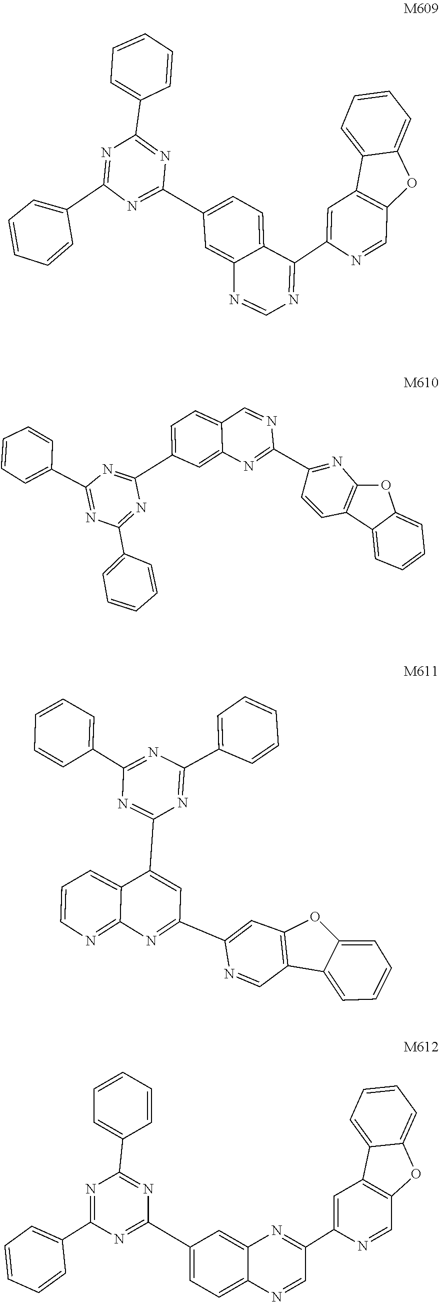 [SMILES: c1ccc(-c2nc(-c3ccccc3)nc(-c3ccc4c(-c5cc6c(cn5)oc5ccccc56)ncnc4c3)n2)cc1 ; c1ccc(-c2nc(-c3ccccc3)nc(-c3ccc4cnc(-c5ccc6c(n5)oc5ccccc56)nc4c3)n2)cc1 ; c1ccc(-c2nc(-c3ccccc3)nc(-c3cc(-c4cc5oc6ccccc6c5cn4)nc4ncccc34)n2)cc1 ; c1ccc(-c2nc(-c3ccccc3)nc(-c3ccc4ncc(-c5cc6c(cn5)oc5ccccc56)nc4c3)n2)cc1]

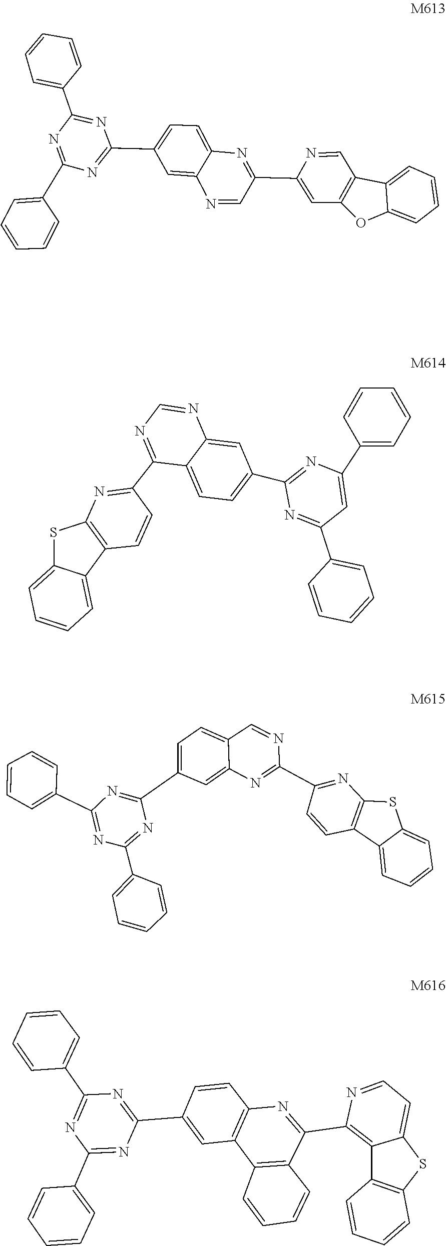 [SMILES: c1ccc(-c2nc(-c3ccccc3)nc(-c3ccc4nc(-c5cc6oc7ccccc7c6cn5)cnc4c3)n2)cc1 ; c1ccc(-c2cc(-c3ccccc3)nc(-c3ccc4c(-c5ccc6c(n5)sc5ccccc56)ncnc4c3)n2)cc1 ; c1ccc(-c2nc(-c3ccccc3)nc(-c3ccc4cnc(-c5ccc6c(n5)sc5ccccc56)nc4c3)n2)cc1 ; c1ccc(-c2nc(-c3ccccc3)nc(-c3ccc4nc(-c5nccc6sc7ccccc7c56)c5ccccc5c4c3)n2)cc1]

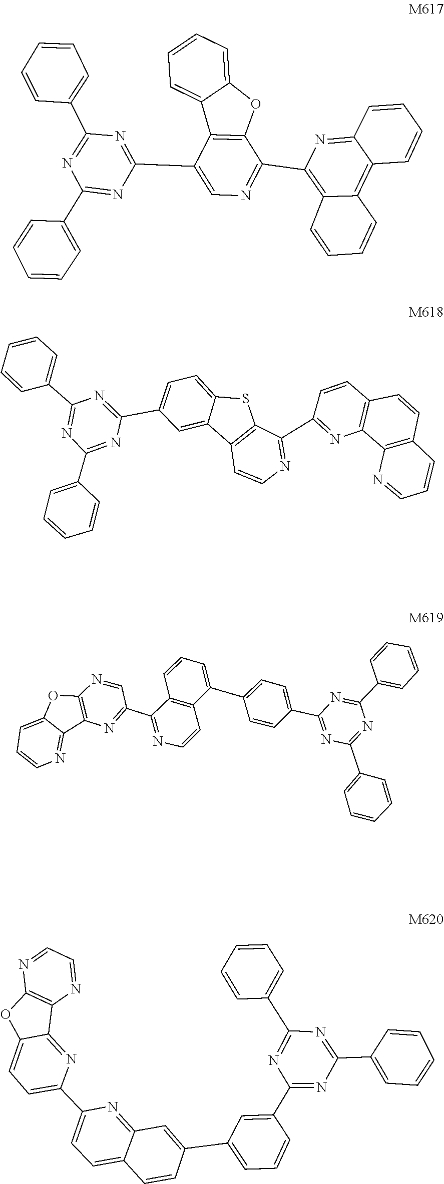 [SMILES: c1ccc(-c2nc(-c3ccccc3)nc(-c3cnc(-c4nc5ccccc5c5ccccc45)c4oc5ccccc5c34)n2)cc1 ; c1ccc(-c2nc(-c3ccccc3)nc(-c3ccc4sc5c(-c6ccc7ccc8cccnc8c7n6)nccc5c4c3)n2)cc1 ; c1ccc(-c2nc(-c3ccccc3)nc(-c3ccc(-c4cccc5c(-c6cnc7oc8cccnc8c7n6)nccc45)cc3)n2)cc1 ; c1ccc(-c2nc(-c3ccccc3)nc(-c3cccc(-c4ccc5ccc(-c6ccc7oc8nccnc8c7n6)nc5c4)c3)n2)cc1]

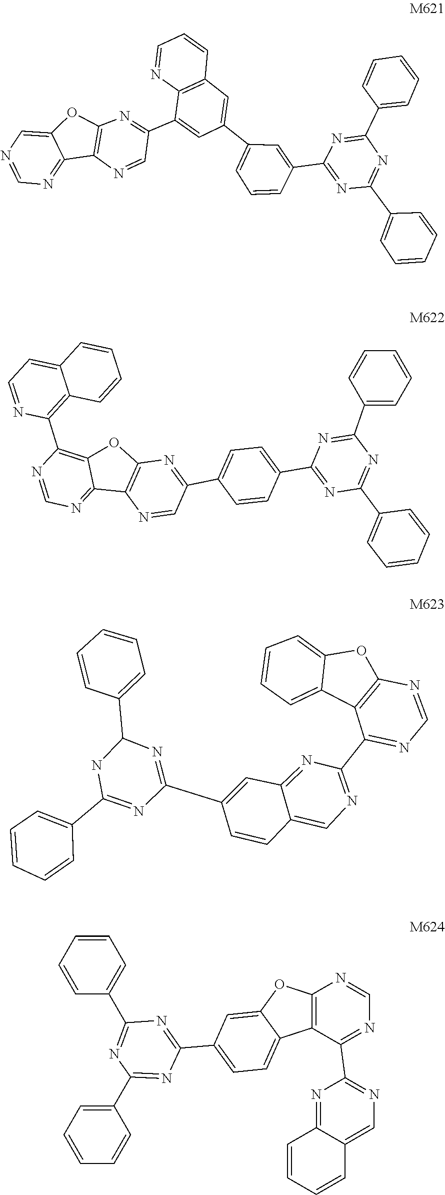 [SMILES: c1ccc(-c2nc(-c3ccccc3)nc(-c3cccc(-c4cc(-c5cnc6c(n5)oc5cncnc56)c5ncccc5c4)c3)n2)cc1 ; c1ccc(-c2nc(-c3ccccc3)nc(-c3ccc(-c4cnc5c(n4)oc4c(-c6nccc7ccccc67)ncnc45)cc3)n2)cc1 ; c1ccc(C2=NC(c3ccc4cnc(-c5ncnc6oc7ccccc7c56)nc4c3)=NC(c3ccccc3)N2)cc1 ; c1ccc(-c2nc(-c3ccccc3)nc(-c3ccc4c(c3)oc3ncnc(-c5ncc6ccccc6n5)c34)n2)cc1]

13. An organic electroluminescent device comprising an anode, a cathode, and an organic thin film layer located between the anode and the cathode, wherein the organic thin film layer comprises an electron transport layer, and the electron transport layer comprises at least one heterocyclic compound having the structure as shown in formula I: 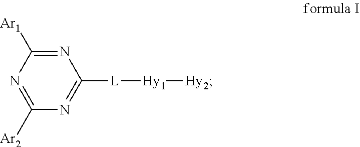
wherein, Ar₁ and Ar₂ are independently selected from the group consisting of hydrogen, deuterium, substituted or unsubstituted C₆-C₁₈ aryl or C₂-C₁₈ heteroaryl;
L is selected from the group consisting of single bond, substituted or unsubstituted C₆-C₃₀ arylene;
Hy₁ and Hy₂ are independently selected from the structures represented by formula II or formula III, and Hy₁ and Hy₂ are different; 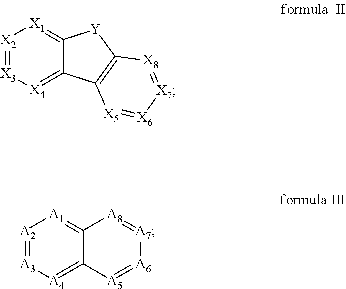
Y is O or S;
X₁, X₂, X₃, X₄, X₅, X₆, X₇, X₈, A₁, A₂, A₃, A₄, A₅, A₆, A₇ and A₈ are independently selected from the group consisting of N atom or CR₁; at least one of X₁, X₂, X₃, X₄, X₅, X₆, X₇, and X₈ is an N atom, and at least one of A₁, A₂, A₃, A₄, A₅, A₆, A₇, and A₈ is an N atom;
R₁ is selected from the group consisting of hydrogen, deuterium, substituted or unsubstituted C₁-C₆ alkyl or C₂-C₆ alkenyl, and adjacent R₁ may be connected to form a ring.

14. An organic electroluminescent device comprising an anode, a cathode, an organic thin film layer located between the anode and the cathode, and an organic cover layer located outside the cathode, wherein the organic cover layer comprises at least one heterocyclic compound having the structure as shown in formula I: 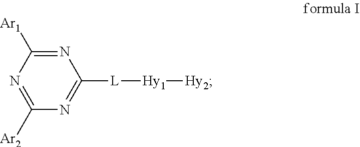
wherein, Ar₁ and Ar₂ are independently selected from the group consisting of hydrogen, deuterium, substituted or unsubstituted C₆-C₁₈ aryl or C₂-C₁₈ heteroaryl;
L is selected from the group consisting of single bond, substituted or unsubstituted C₆-C₃₀ arylene;
Hy₁ and Hy₂ are independently selected from the structures represented by formula II or formula III, and Hy₁ and Hy₂ are different; 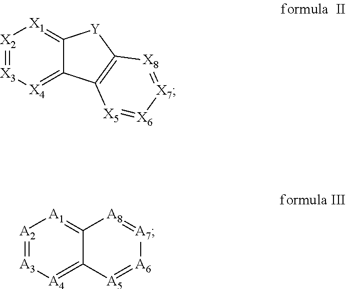
Y is O or S;
X₁, X₂, X₃, X₄, X₅, X₆, X₇, X₈, A₁, A₂, A₃, A₄, A₅, A₆, A₇ and A₈ are independently selected from the group consisting of N atom or CR₁; at least one of X₁, X₂, X₃, X₄, X₅, X₆, X₇, and X₈ is an N atom, and at least one of A₁, A₂, A₃, A₄, A₅, A₆, A₇, and A₈ is an N atom;
R₁ is selected from the group consisting of hydrogen, deuterium, substituted or unsubstituted C₁-C₆ alkyl or C₂-C₆ alkenyl, and adjacent R₁ may be connected to form a ring.

15. A display device comprising the organic electroluminescent device according to claim 13.

16. A display device comprising the organic electroluminescent device according to claim 14.
